(19) United States
(12) Patent Application Publication
(10) Pub. No.: US 2025/0339384 A1
(43) Pub. Date: Nov. 6, 2025

(54) MODIFIED RHABDOVIRUS GLYCOPROTEINS AND USES THEREOF

(71) Applicants: Regeneron Pharmaceuticals, Inc., Tarrytown, NY (US); Vyriad, Inc., Rochester, MN (US)

(72) Inventors: Jason Buehler, Rochester, MN (US); Stephen J. Russell, Rochester, MN (US); Gopal Naik Nenavath, Rochester, MN (US); Melissa Moy, Rochester, MN (US); Chris Ziegler, Rochester, MN (US); Rianna Vandergaast, Rochester, MN (US)

(73) Assignees: Regeneron Pharmaceuticals, Inc., Tarrytown, NY (US); Vyriad, Inc., Rochester, MN (US)

(21) Appl. No.: 19/151,008

(22) PCT Filed: Jan. 26, 2024
(86) PCT No.: PCT/US2024/013058

§ 371 Date: Jul. 25, 2025

Related U.S. Application Data
(60) Provisional application No. 63/481,847, filed on Jan. 27, 2023.
(60) Provisional application No. 63/466,931, filed on May 16, 2023.
(60) Provisional application No. 63/597,831, filed on Nov. 10, 2023.

(51) Int. Cl. A61K 9/51; C07K 14/005; C07K 16/28; C07K 16/32; C12N 9/22; C12N 15/11

(52) U.S. Cl. CPC A61K 9/5184; C07K 14/005; C07K 16/2863; C07K 16/32; C12N 9/226; C12N 15/111; C07K 2317/622; C07K 2319/50; C12N 2310/20; C12N 2740/15022; C12N 2740/15042; C12N 2760/20222; C12N 2760/20242

(57) ABSTRACT

The present disclosure provides recombinant fusogenic proteins comprising a rhabdovirus glycoprotein (G) and a targeting molecule attached to the N-terminus of the rhabdovirus glycoprotein. Further provided are related recombinant polynucleotides, host cells, and pharmaceutical compositions. Recombinant viruses, e.g., recombinant pseudotyped viruses, and cell-derived nanovesicles comprising the recombinant polynucleotides are also provided. Further provided are methods for using the recombinant fusogenic proteins, polynucleotides, viruses, and cell-derived nanovesicles, and/or pharmaceutical compositions thereof, including their use in the treatment of cancer.

### CROSS-REFERENCE TO RELATED APPLICATIONS

This application claims priority to U.S. Provisional Application No. 63/481,847, filed Jan. 27, 2023, U.S. Provisional Application No. 63/466,931, filed May 16, 2023, and U.S. Provisional Application No. 63/597,831, filed Nov. 10, 2023, the disclosure each of which is herein incorporated by reference in its entirety.

### SEQUENCE LISTING

The instant application contains a Sequence Listing which has been submitted electronically in XML format and is hereby incorporated by reference in its entirety. Said XML copy, created on Jan. 19, 2024, is named 250298_000603_SL.xml and is 266,253 bytes in size.

### FIELD OF THE INVENTION

The present disclosure provides recombinant fusogenic proteins comprising a rhabdovirus glycoprotein (G) and a targeting molecule attached to the N-terminus of the rhabdovirus glycoprotein. Further provided are related recombinant polynucleotides, host cells, and pharmaceutical compositions. Recombinant viruses, e.g., recombinant pseudotyped viruses, and cell-derived nanovesicles comprising the recombinant polynucleotides are also provided. Further provided are methods for using the recombinant fusogenic proteins, polynucleotides, viruses, and cell-derived nanovesicles, and/or pharmaceutical compositions thereof, including their use in the treatment of cancer.

### BACKGROUND OF THE INVENTION

A significant challenge in developing targeted vector therapies is delivery of the therapeutic to cells/tissues specific to a target disease. To achieve success, delivered vector therapies must be specific to the target cells/tissues and reduce any off-target delivery that could cause toxicity. Additionally, therapeutic vectors must overcome natural barriers, such as patient innate immune responses, long enough to reach target cells/tissues.

Vesicular stomatitis virus (VSV) has oncolytic properties and clinical trials are underway to determine its safety and efficacy as an anti-cancer therapy. Because VSV-G glycoprotein is known to infect a very broad range of cells and tissues (Finkelshtein et al., 2013; Nikolic et al., 2018), an ongoing challenge is to improve targeted delivery of VSVs to tumor tissues to maximize therapeutic effect and to minimize potential toxicity associated with infection of healthy tissues. One of the most concerning off-target effects that has been associated with VSV is neurotoxicity. Loss of virus into other tissues, including the liver and spleen, decreases the efficacy of oncolytic VSV. Strategies to direct tropism of VSV to tissues of interest, have frequently resulted in negative impacts on virus fitness, which also decreased the efficacy of VSV therapy.

Accordingly, there is an unmet need in the art for improved VSV-based therapies.

### SUMMARY OF THE INVENTION

As specified in the Background section above, there is a great need in the art for improved VSV-based therapies. The present application addresses these and other needs.

In one aspect, provided herein is a recombinant fusogenic protein, wherein said fusogenic protein comprises:
(i) a rhabdovirus glycoprotein (G), or a functional fragment or derivative thereof; and
(ii) a targeting molecule, wherein said targeting molecule can be attached to, e.g., the N-terminus of said rhabdovirus glycoprotein, or the functional fragment or derivative thereof, via a linker, said linker being sensitive to a proteolytic cleavage by an endogenous protease or by an exogenously added protease.

In some embodiments, said linker comprises an Arginine (R) and/or Lysine (K) residue.

In some embodiments, said linker is comprised within the sequence selected from:

(SEQ ID NO: 174)
KRAAASGGS(G₄S)₂GPK;
(SEQ ID NO: 2)
KRAAASGGS(G₄S)₂;
(SEQ ID NO: 3)
(EAAAK)₃;
(SEQ ID NO: 4)
KR(EAAAK)₃;
(SEQ ID NO: 5)
AAARGSPK(G₄S)₃;
(SEQ ID NO: 169)
RAAARGSPK(G₄S)₃;
(SEQ ID NO: 19)
AAARGSPK(G₄S)₃K;
(SEQ ID NO: 20)
K(G₄S)₃;
(SEQ ID NO: 21)
KR(G₄S)₃;
(SEQ ID NO: 6)
(G₄S)₃GPK;
and
(SEQ ID NO: 7)
AAA(G₄S)₃K.

In some embodiments, the N-terminus of the rhabdovirus glycoprotein, or the functional fragment or derivative thereof, to which the targeting molecule is attached, via a linker, does not comprise one or more amino acids present at the N-terminus of a mature wild-type rhabdovirus glycoprotein.

In some embodiments, said rhabdovirus glycoprotein is a vesicular stomatitis virus glycoprotein (VSV-G), or a functional fragment or derivative thereof.

In some embodiments, said VSV-G comprises the sequence SEQ ID NO: 8.

In some embodiments, said VSV-G consists of the sequence SEQ ID NO: 8.

In some embodiments, the targeting molecule is capable of interfering with the ability of said VSV-G, or the functional fragment or derivative thereof, to interact with low-density lipoprotein receptor (LDLR).

In some embodiments, said VSV-G, or the functional fragment or derivative thereof, comprises one or more mutations, wherein the one or more mutations reduces or eliminates binding of said VSV-G polypeptide, or the functional fragment or derivative thereof, to LDLR.

In some embodiments, said one or more mutations in said VSV-G, or the functional fragment or derivative thereof, comprise one or more amino acid substitutions and/or deletions at positions corresponding to H8, K47, Y209, or R354 in SEQ ID NO: 8.

In some embodiments, said VSV-G comprises or consists of SEQ ID NO: 8, and said one or more mutations are substitutions at positions K47 and R354.

In some embodiments, said VSV-G comprises or consists of SEQ ID NO: 8, and said one or more mutations are substitutions at positions K47, R354 and Y209.

In some embodiments, said VSV-G comprises or consists of SEQ ID NO: 8, and said one or more mutations is a substitution at position H8.

In some embodiments, said one or more mutations in said VSV-G, or the functional fragment or derivative thereof, comprise one or more amino acid deletions at positions corresponding to H8, K47, Y209, or R354 in SEQ ID NO: 8.

In some embodiments, said VSV-G comprises or consists of SEQ ID NO: 8.

In some embodiments, said one or more deletions is a deletion at position K47.

In some embodiments, said one or more deletions is a deletion at position H8.

In some embodiments, said one or more deletions are deletions at positions H8 and K47.

In some embodiments, said VSV-G, or the functional fragment or derivative thereof, further comprises one or more viral titer increasing mutations.

In some embodiments, said one or more viral titer increasing mutations in said VSV-G, or the functional fragment or derivative thereof, is M184T and/or F250L, as specified relative to positions in SEQ ID NO: 8.

In some embodiments, said rhabdovirus glycoprotein is a glycoprotein from Flanders virus (FLAV-G).

In some embodiments, said FLAV-G comprises the sequence SEQ ID NO: 9.

In some embodiments, said FLAV-G consists of the sequence SEQ ID NO: 9.

In some embodiments, said rhabdovirus glycoprotein is a glycoprotein from Chandipura virus (CHPV-G).

In some embodiments, said CHPV-G comprises the sequence SEQ ID NO: 10.

In some embodiments, said CHPV-G consists of the sequence SEQ ID NO: 10.

In some embodiments, said rhabdovirus glycoprotein is a glycoprotein from Perinet virus (PERV-G).

In some embodiments, said PERV-G comprises the sequence SEQ ID NO: 11.

In some embodiments, said PERV-G consists of the sequence SEQ ID NO: 11.

In some embodiments, said rhabdovirus glycoprotein is a glycoprotein from Piry virus (PIRYV-G).

In some embodiments, said PIRYV-G comprises the sequence SEQ ID NO: 12.

In some embodiments, said PIRYV-G consists of the sequence SEQ ID NO: 12.

In some embodiments, said rhabdovirus glycoprotein is a glycoprotein from Fukuoka virus (FUKV-G).

In some embodiments, said FUKV-G comprises the sequence SEQ ID NO: 13.

In some embodiments, said FUKV-G consists of the sequence SEQ ID NO: 13.

In some embodiments, said rhabdovirus glycoprotein is a glycoprotein from Joinjakaka virus (JOIV-G).

In some embodiments, said JOIV-G comprises the sequence SEQ ID NO: 14.

In some embodiments, said JOIV-G consists of the sequence SEQ ID NO: 14.

In some embodiments, said rhabdovirus glycoprotein is a glycoprotein from Kumasi virus (KRV-G).

In some embodiments, said KRV-G comprises the sequence SEQ ID NO: 15.

In some embodiments, said KRV-G consists of the sequence SEQ ID NO: 15.

In some embodiments, said rhabdovirus glycoprotein is a glycoprotein from Keuraliba virus (KEUV-G).

In some embodiments, said KEUV-G comprises the sequence SEQ ID NO: 17.

In some embodiments, said KEUV-G comprises the sequence SEQ ID NO: 17.

In some embodiments, the cytoplasmic tail of the rhabdovirus glycoprotein has been removed or truncated, and optionally replaced with another sequence.

In some embodiments, the cytoplasmic tail of the glycoprotein is truncated by up to 40 amino acids from the C-terminus.

In some embodiments, the cytoplasmic tail of the rhabdovirus glycoprotein is truncated by 10 to 40 amino acids from the C-terminus.

In some embodiments, the cytoplasmic tail of the rhabdovirus glycoprotein is truncated by 30 amino acids from the C-terminus.

In some embodiments of any of the above-described recombinant fusogenic proteins, the recombinant fusogenic protein may further comprise a cytoplasmic tail from VSV-G, or a functional fragment or derivative thereof.

In some embodiments, the cytoplasmic tail of VSV-G comprises the sequence CIKLKHTKKRQIYTDIEMNRLGK (SEQ ID NO: 16).

In another aspect, provided herein is a recombinant fusogenic protein, wherein said fusogenic protein comprises a fusogen that has at least 60% amino acid sequence identity to vesicular stomatitis virus glycoprotein (VSV-G) comprising SEQ ID NO: 8, or a functional fragment or derivative thereof, wherein said fusogen, or the functional fragment or derivative thereof, comprises one or more amino acid deletions at positions corresponding to H8, K47, Y209, or R354 in SEQ ID NO: 8.

In some embodiments, said fusogen comprises the sequence SEQ ID NO: 8, or a functional fragment or derivative thereof, with one or more amino acid deletions at positions H8, K47, Y209, or R354.

In some embodiments, said fusogen comprises or consists of the sequence SEQ ID NO: 8, with an amino acid deletion at position H8.

In some embodiments, said fusogen comprises or consists of the sequence SEQ ID NO: 8, with amino acid deletions at positions H8 and K47.

In some embodiments, said fusogen comprises the sequence SEQ ID NO: 8, with amino acid deletions at positions (i) K47, (ii) R354, and (iii) H8 or Y209.

In some embodiments, said fusogen consists of the sequence SEQ ID NO: 8, with amino acid deletions at positions (i) K47, (ii) R354, and (iii) H8 or Y209.

In some embodiments, said fusogen comprises the sequence SEQ ID NO: 8, with an amino acid deletion at position K47.

In some embodiments, said fusogen consists of the sequence SEQ ID NO: 8, with an amino acid deletion at positions K47.

In another aspect, provided herein is a recombinant fusogenic protein that comprises a fusogen that comprises the sequence SEQ ID NO: 8, with amino acid substitutions at positions (i) K47, (ii) R354, and (iii) H8 or Y209.

In some embodiments, said fusogen consists of the sequence SEQ ID NO: 8, with amino acid substitutions at positions (i) K47, (ii) R354, and (iii) H8 or Y209.

In another aspect, provided herein is a recombinant fusogenic protein that comprises a fusogen that comprises the sequence SEQ ID NO: 8, with amino acid substitutions at positions K47, R354, H8, and Y209.

In some embodiments, said fusogen consists of the sequence SEQ ID NO: 8, with amino acid substitutions at positions K47, R354, H8, and Y209.

In some embodiments, said fusogen, or the functional fragment or derivative thereof, further comprises one or more viral titer increasing mutations.

In some embodiments, said one or more viral titer increasing mutations are in one or more positions corresponding to positions M184 and/or F250 in SEQ ID NO: 8.

In some embodiments of any of the above-described recombinant fusogenic proteins, the fusogenic protein may further comprise a targeting molecule located at the N-terminus of said fusogen, or the functional fragment or derivative thereof.

In some embodiments, said targeting molecule is attached to the N-terminus of said fusogen, or the functional fragment or derivative thereof, via a linker.

In some embodiments, said linker is sensitive to a proteolytic cleavage by an endogenous protease or by an exogenously added protease.

In some embodiments, said linker comprises an Arginine (R) and/or Lysine (K) residue.

In some embodiments, said linker is comprised within the sequence selected from:

(SEQ ID NO: 174)
KRAAASGGS(G₄S)₂GPK;
(SEQ ID NO: 2)
KRAAASGGS(G₄S)₂;
(SEQ ID NO: 3)
(EAAAK)₃;
(SEQ ID NO: 4)
KR(EAAAK)₃;
(SEQ ID NO: 5)
AAARGSPK(G₄S)₃;
(SEQ ID NO: 169)
RAAARGSPK(G₄S)₃;
(SEQ ID NO: 19)
AAARGSPK(G₄S)₃K;
(SEQ ID NO: 20)
K(G₄S)₃;
(SEQ ID NO: 21)
KR(G₄S)₃;
(SEQ ID NO: 6)
(G₄S)₃GPK;
and
(SEQ ID NO: 7)
AAA(G₄S)₃K.

In some embodiments, said linker is not sensitive to a proteolytic cleavage by an endogenous protease or by an exogenously added protease.

In some embodiments, the N-terminus of the fusogen, or the functional fragment or derivative thereof, to which the targeting molecule is attached, does not comprise one or more amino acids present at the N-terminus of a mature wild-type fusogen.

In another aspect, provided herein is a recombinant fusogenic protein, wherein said fusogenic protein comprises:
(i) a glycoprotein from Flanders virus (FLAV-G), or a functional fragment or derivative thereof, and
(ii) a targeting molecule.

In some embodiments, said FLAV-G comprises the sequence SEQ ID NO: 9.

In some embodiments, said FLAV-G consists of the sequence SEQ ID NO: 9.

In another aspect, provided herein is recombinant fusogenic protein, wherein said fusogenic protein comprises:
(i) a glycoprotein from Chandipura virus (CPV-G), or a functional fragment or derivative thereof, and
(ii) a targeting molecule.

In some embodiments, said CHPV-G comprises the sequence SEQ ID NO: 10.

In some embodiments, said CHPV-G consists of the sequence SEQ ID NO: 10.

In another aspect, provided herein is a recombinant fusogenic protein, wherein said fusogenic protein comprises:
(i) a glycoprotein from Perinet virus (PERV-G), or a functional fragment or derivative thereof, and
(ii) a targeting molecule.

In some embodiments, said PERV-G comprises the sequence SEQ ID NO: 11.

In some embodiments, wherein said PERV-G consists of the sequence SEQ ID NO: 11.

In another aspect, provided herein is a recombinant fusogenic protein, wherein said fusogenic protein comprises:
(i) a glycoprotein from Piry virus (PIRYV-G), or a functional fragment or derivative thereof, and
(ii) a targeting molecule.

In some embodiments, said PIRYV-G comprises the sequence SEQ ID NO: 12.

In some embodiments, said PIRYV-G consists of the sequence SEQ ID NO: 12.

In another aspect, provided herein is a recombinant fusogenic protein, wherein said fusogenic protein comprises:
(i) a glycoprotein from Fukuoka virus (FUKV-G), or a functional fragment or derivative thereof, and
(ii) a targeting molecule.

In some embodiments, said FUKV-G comprises the sequence SEQ ID NO: 13.

In some embodiments, said FUKV-G consists of the sequence SEQ ID NO: 13.

In another aspect, provided herein is a recombinant fusogenic protein, wherein said fusogenic protein comprises:
(i) a glycoprotein from Joinjakaka virus (JOIV-G), or a functional fragment or derivative thereof, and
(ii) a targeting molecule.

In some embodiments, said JOIV-G comprises the sequence SEQ ID NO: 14.

In some embodiments, said JOIV-G consists of the sequence SEQ ID NO: 14.

In another aspect, provided herein is a recombinant fusogenic protein, wherein said fusogenic protein comprises:
(i) a glycoprotein from Kumasi virus (KRV-G), or a functional fragment or derivative thereof, and
(ii) a targeting molecule.

In some embodiments, said KRV-G comprises the sequence SEQ ID NO: 15.

In some embodiments, said KRV-G consists of the sequence SEQ ID NO: 15.

In another aspect, provided here is a recombinant fusogenic protein, wherein said fusogenic protein comprises:
(i) a glycoprotein from Keuraliba virus (KEUV-G), or a functional fragment or derivative thereof, and
(ii) a targeting molecule.

In some embodiments, said KEUV-G comprises the sequence SEQ ID NO: 17.

In some embodiments, said KEUV-G consists of the sequence SEQ ID NO: 17.

In some embodiments, said glycoprotein is a fragment, wherein the cytoplasmic tail of the glycoprotein has been removed or truncated, and optionally replaced with another sequence.

In some embodiments, the cytoplasmic tail of the glycoprotein is truncated by up to 40 amino acids from the C-terminus.

In some embodiments, the cytoplasmic tail of the glycoprotein is truncated by 10 to 40 amino acids from the C-terminus.

In some embodiments, the cytoplasmic tail of the glycoprotein is truncated by 30 amino acids from the C-terminus.

In some embodiments of any of the above-described recombinant fusogenic proteins, the recombinant fusogenic protein may further comprise a cytoplasmic tail from VSV-G, or a functional fragment or derivative thereof.

In some embodiments, the cytoplasmic tail of VSV-G comprises the sequence CIKLKHTKKRQIYTDIEMNRLGK (SEQ ID NO: 16).

In some embodiments, the targeting molecule is located at the N-terminus of said glycoprotein, or the functional fragment or derivative thereof.

In some embodiments, said targeting molecule is attached to the N-terminus of said glycoprotein, or the functional fragment or derivative thereof, via a linker.

In some embodiments, said linker is sensitive to a proteolytic cleavage by an endogenous protease or by an exogenously added protease.

In some embodiments, said linker comprises an Arginine (R) and/or Lysine (K) residue.

In some embodiments, said linker is comprised within the sequence selected from:

(SEQ ID NO: 174)
KRAAASGGS(G₄S)₂GPK;
(SEQ ID NO: 2)
KRAAASGGS(G₄S)₂;
(SEQ ID NO: 3)
(EAAAK)₃;
(SEQ ID NO: 4)
KR(EAAAK)₃;
(SEQ ID NO: 5)
AAARGSPK(G₄S)₃;
(SEQ ID NO: 169)
RAAARGSPK(G₄S)₃;
(SEQ ID NO: 19)
AAARGSPK(G₄S)₃K;
(SEQ ID NO: 20)
K(G₄S)₃;
(SEQ ID NO: 21)
KR(G₄S)₃;
(SEQ ID NO: 6)
(G₄S)₃GPK;
and
(SEQ ID NO: 7)
AAA(G₄S)₃K.

In some embodiments, said linker is not sensitive to a proteolytic cleavage by an endogenous protease or by an exogenously added protease.

In some embodiments, the N-terminus of the glycoprotein, or the functional fragment or derivative thereof, to which the targeting molecule is attached, does not comprise one or more amino acids present at the N-terminus of a mature wild-type fusogen.

In some embodiments, said targeting molecule is an antibody or antigen-binding fragment thereof, an affibody, a darpin, a peptide, a natural or modified natural receptor ligand, a T cell receptor or a fragment or derivative thereof, or an MHC-peptide complex or a fragment or derivative thereof.

In some embodiments, said antibody or antigen-binding fragment thereof is a single-chain fragment variable (scFv), a diabody, a minibody, a nanobody, a single-domain antibody (sdAb), or a single heavy chain antibody.

In some embodiments, said targeting molecule targets EGFR, HER2, MUC16, cKit, αVβ3 Integrin, IGF1R, BCMA, Nectin-4, MEK, CD44, CD3, CD4, CD28, stem cell factor, thrombopoietin, c-Met, CXCR4, IL2R, or IL-3.

In another aspect, provided herein is a recombinant polynucleotide encoding a recombinant fusogenic protein described herein.

In some embodiments, the polynucleotide comprises a sequence encoding a signal peptide sequence, wherein such signal sequence is positioned at the extreme N-terminus of the encoded recombinant fusogenic protein.

In some embodiments, the polynucleotide is DNA.

In some embodiments, the polynucleotide is RNA.

In another aspect, provided herein is a recombinant polynucleotide, wherein the recombinant polynucleotide is an RNA molecule comprising a nucleotide sequence that is a template for a positive sense transcript encoding a recombinant fusogenic protein described herein.

In some embodiments, the positive sense transcript comprises a sequence encoding a signal peptide sequence, wherein such signal sequence is positioned at the extreme N-terminus of the encoded recombinant fusogenic protein.

In some embodiments, the recombinant polynucleotide is an RNA molecule comprising a nucleotide sequence that is a template for a positive sense transcript encoding a vesicular stomatitis virus (VSV) nucleoprotein (N) polypeptide or a functional fragment or derivative thereof, a nucleotide sequence that is a template for a positive sense transcript encoding a VSV phosphoprotein (P) polypeptide or a functional fragment or derivative thereof, a nucleotide sequence that is a template for a positive sense transcript encoding a VSV matrix (M) polypeptide or a functional fragment or derivative thereof, a nucleotide sequence that is a template for a positive sense transcript encoding a fusogenic protein described herein, and a nucleotide sequence that is a template for a positive sense transcript encoding a VSV large protein (L) polypeptide or a functional fragment or derivative thereof.

In some embodiments, said VSV M polypeptide is a mutant VSV M polypeptide.

In some embodiments, said mutant VSV M polypeptide comprises a mutation at methionine (M) 51.

In some embodiments, said mutation at methionine (M) 51 is a substitution from methionine (M) to arginine (R).

In some embodiments, said polynucleotide is optimized for expression in human cells.

In another aspect, provided herein is a composition comprising a recombinant polynucleotide described herein and a carrier and/or excipient.

In another aspect, provided herein is a host cell comprising a recombinant polynucleotide described herein.

In another aspect, provided herein is a recombinant pseudotyped virus or cell-derived nanovesicle comprising a recombinant polynucleotide described herein.

In another aspect, provided herein is a recombinant pseudotyped virus or cell-derived nanovesicle comprising one or more recombinant fusogenic proteins described herein.

In some embodiments, a recombinant pseudotyped virus or cell-derived nanovesicle described herein, may comprise two or more different recombinant fusogenic proteins described herein.

In some embodiments, said recombinant fusogenic protein forms a chimeric trimer with one or two different fusogenic proteins on the surface of said recombinant pseudotyped virus or cell-derived nanovesicle.

In some embodiments, said chimeric trimer comprises (i) at least one fusogenic protein described herein and (ii) a fusogenic protein comprising a rhabdoviral glycoprotein, or a functional fragment or derivative thereof, without a targeting molecule.

In some embodiments, the fusogenic protein (ii) comprises a fusogen that comprises the sequence SEQ ID NO: 8, with amino acid substitutions and/or deletions at one or more positions selected from K47, R354, H8, and Y209.

In another aspect, provided herein is a recombinant pseudotyped virus or cell-derived nanovesicle comprising a chimeric trimer comprising (i) one or two monomers of a first fusogenic protein, wherein said first fusogenic protein comprises a rhabdovirus glycoprotein, or a functional fragment or derivative thereof; and a targeting molecule, or the functional fragment or derivative thereof, and (ii) one or two monomers of a second fusogenic protein, wherein said second fusogenic protein comprises a rhabdovirus glycoprotein, or a functional fragment or derivative thereof, without a targeting molecule. In some embodiments, for example, the targeting molecule can be attached to the N-terminus of the rhabdovirus glycoprotein, of functional fragment or derivative thereof.

In some embodiments, in the first fusogenic protein, the targeting molecule is attached to the rhabdovirus glycoprotein via a linker.

In some embodiments, said linker is not sensitive to a proteolytic cleavage by an endogenous protease or by an exogenously added protease.

In some embodiments, said linker is sensitive to a proteolytic cleavage by an endogenous protease or by an exogenously added protease.

In some embodiments, the first fusogenic protein and/or the second fusogenic protein comprises a rhabdovirus glycoprotein that comprises the sequence SEQ ID NO: 8, with amino acid substitutions and/or deletions at one or more positions selected from K47, R354, H8, and Y209.

In some embodiments, the first fusogenic protein and/or the second fusogenic protein comprises a rhabdovirus glycoprotein that comprises any of various rhabdovirus glycoprotein sequences set forth herein. In some embodiments, the first fusogenic protein and/or the second fusogenic protein comprises a rhabdovirus glycoprotein that comprises one or more amino acid substitutions and/or deletions at positions corresponding to H8, K47, Y209, or R354 in SEQ ID NO: 8. In some embodiments, the first fusogenic protein and/or the second fusogenic protein comprises a rhabdovirus glycoprotein that comprises or consists of SEQ ID NO: 8, and said one or more amino acid mutations are substitutions at positions K47 and R354. In some embodiments, said one or more amino acid mutations are substitutions at positions K47, R354 and Y209. In some embodiments, said one or more amino acid mutations is a substitution at position H8.

In some embodiments, the first fusogenic protein and/or the second fusogenic protein comprises a rhabdovirus glycoprotein that comprises one or more amino acid deletions at positions corresponding to H8, K47, Y209, or R354 in SEQ ID NO: 8. In some embodiments, the first fusogenic protein and/or the second fusogenic protein comprises a rhabdovirus glycoprotein that comprises or consists of the sequence SEQ ID NO: 8, and said one or more amino acid deletions are deletions at positions H8, K47, Y209, or R354. In some embodiments, said one or more amino acid deletions is a deletion at position K47. In some embodiments, said one or more amino acid deletions is a deletion at position H8. In some embodiments, said one or more amino acid deletions are deletions at positions H8 and K47.

In another aspect, provided herein is a recombinant pseudotyped virus or cell-derived nanovesicle comprising a glycoprotein from Flanders virus (FLAV-G), or a functional fragment or derivative thereof.

In some embodiments, said FLAV-G comprises the sequence SEQ ID NO: 9.

In some embodiments, said FLAV-G consists of the sequence SEQ ID NO: 9.

In another aspect, provided herein is a recombinant pseudotyped virus or cell-derived nanovesicle comprising a glycoprotein from Chandipura virus (CHPV-G), or a functional fragment or derivative thereof.

In some embodiments, said CHPV-G comprises the sequence SEQ ID NO: 10.

In some embodiments, said CHPV-G consists of the sequence SEQ ID NO: 10.

In another aspect, provided herein is a recombinant pseudotyped virus or cell-derived nanovesicle comprising a glycoprotein from Perinet virus (PERV-G), or a functional fragment or derivative thereof.

In some embodiments, said PERV-G comprises the sequence SEQ ID NO: 11.

In some embodiments, said PERV-G consists of the sequence SEQ ID NO: 11.

In another aspect, provided herein is a recombinant pseudotyped virus or cell-derived nanovesicle comprising a glycoprotein from Piry virus (PIRYV-G), or a functional fragment or derivative thereof.

In some embodiments, said PIRYV-G comprises the sequence SEQ ID NO: 12.

In some embodiments, said PIRYV-G consists of the sequence SEQ ID NO: 12.

In another aspect, provided herein is a recombinant pseudotyped virus or cell-derived nanovesicle comprising a glycoprotein from Fukuoka virus (FUKV-G), or a functional fragment or derivative thereof.

In some embodiments, said FUKV-G comprises the sequence SEQ ID NO: 13.

In some embodiments, said FUKV-G consists of the sequence SEQ ID NO: 13.

In another aspect, provided herein is a recombinant pseudotyped virus or cell-derived nanovesicle comprising a glycoprotein from Joinjakaka virus (JOIV-G), or a functional fragment or derivative thereof.

In some embodiments, said JOIV-G comprises the sequence SEQ ID NO: 14.

In some embodiments, said JOIV-G consists of the sequence SEQ ID NO: 14.

In another aspect, provided herein is a recombinant pseudotyped virus or cell-derived nanovesicle comprising a glycoprotein from Kumasi virus (KRV-G), or a functional fragment or derivative thereof.

In some embodiments, said KRV-G comprises the sequence SEQ ID NO: 15.

In some embodiments, said KRV-G consists of the sequence SEQ ID NO: 15.

In another aspect, provided herein is a recombinant pseudotyped virus or cell-derived nanovesicle comprising a glycoprotein from Keuraliba virus (KEUV-G), or a functional fragment or derivative thereof.

In some embodiments, said KEUV-G comprises the sequence SEQ ID NO: 17.

In some embodiments, said KEUV-G consists of the sequence SEQ ID NO: 17.

In some embodiments, the cytoplasmic tail of said glycoprotein has been removed or truncated, and optionally replaced with another sequence.

In some embodiments, the cytoplasmic tail of the glycoprotein is truncated by up to 40 amino acids from the C-terminus.

In some embodiments, the cytoplasmic tail of the glycoprotein is truncated by 10 to 40 amino acids from the C-terminus.

In some embodiments, the cytoplasmic tail of the glycoprotein is truncated by 30 amino acids from the C-terminus.

In some embodiments, said glycoprotein further comprises a cytoplasmic tail from VSV-G, or a functional fragment or derivative thereof.

In some embodiments, the cytoplasmic tail of VSV-G comprises the sequence CIKLKHTKKRQIYTDIEMNRLGK (SEQ ID NO: 16).

In some embodiments, said virus is a rhabdovirus.

In some embodiments, said virus is a recombinant vesicular stomatitis virus (VSV).

In some embodiments, said virus is a retrovirus.

In some embodiments, said retrovirus is a lentivirus (LV).

In some embodiments, said virus is replication-competent.

In some embodiments, said virus is non-replicative.

In some embodiments, said virus further comprises a molecular cargo.

In some embodiments, said molecular cargo is a transgene encoding a therapeutic protein, a suicide gene, a toxic protein or peptide, an antibody or a fragment thereof, a chimeric antigen receptor (CAR), a T cell receptor (TCR), a gene editing system or a component(s) thereof, an antisense oligonucleotide, a ribozyme, or an RNAi molecule.

In some embodiments, said molecular cargo is a therapeutic protein, a toxic protein or peptide, an antibody or a fragment thereof, a chimeric antigen receptor (CAR), a T cell receptor (TCR), a gene editing system or a component(s) thereof, an antisense oligonucleotide, a ribozyme, or an RNAi molecule.

In some embodiments, said molecular cargo is a gene editing ribonucleoprotein complex or a component(s) thereof.

In some embodiments, said molecular cargo is Cas9 protein complexed with a guide RNA (gRNA) specific to a gene of interest.

In another aspect, provided herein is a composition comprising a recombinant pseudotyped virus or cell-derived nanovesicle described herein, and a carrier and/or excipient.

In another aspect, provided herein is a method of decreasing susceptibility to serum neutralization of a recombinant virus or nanovesicle in a subject in need thereof, comprising administering to the subject a recombinant pseudotyped virus or cell-derived nanovesicle described herein or a composition described herein.

In another aspect, provided herein is a method of enhancing resistance to low-density lipoprotein (LDL)- and/or very-low-density lipoprotein (VLDL)-mediated neutralization in a subject in need thereof, comprising administering to the subject a recombinant pseudotyped virus or cell-derived nanovesicle described herein or a composition described herein.

In another aspect, provided herein is a method of treating a cancer in a subject in need thereof, comprising administering to the subject a therapeutically effective amount of a recombinant pseudotyped virus or cell-derived nanovesicle described herein or a composition described herein.

In some embodiments, said method does not include pre-treatment with LDL/VLDL-lowering medications.

In some embodiments, said method further comprises pre-treatment with LDL/VLDL-lowering medications.

In another aspect, provided herein is a method of inducing an immune response in a subject in need thereof, comprising administering to the subject an effective amount of a recombinant pseudotyped virus or cell-derived nanovesicle described herein or a composition described herein.

In another aspect, provided herein is a method for delivering a molecular cargo to a cell within a subject in need thereof, comprising administering to the subject an effective amount of a recombinant pseudotyped virus or cell-derived nanovesicle described herein, or a composition comprising said pseudotyped virus or cell-derived nanovesicle and a carrier and/or excipient, wherein the recombinant fusogenic protein within said recombinant pseudotyped virus or cell-derived nanovesicle comprises a targeting molecule which targets said cell.

In some embodiments, the subject is human.

In another aspect, provided herein is a method for delivering a molecular cargo to a cell ex vivo, comprising administering to said cell an effective amount of a recombinant pseudotyped virus or cell-derived nanovesicle described herein, or a composition comprising said pseudotyped virus or cell-derived nanovesicle and a carrier and/or excipient, wherein the recombinant fusogenic protein within said recombinant pseudotyped virus or cell-derived nanovesicle comprises a targeting molecule which targets said cell.

### BRIEF DESCRIPTION OF THE DRAWINGS

FIGS. 1A-1B illustrate that serum inhibits wild-type (WT) vesicular stomatitis virus (VSV). A WT (glycoprotein [G] containing) VSV (VSV-G) encoding a firefly luciferase (Fluc) reporter was used to infect Vero cells. Increasing concentrations of pooled human serum were added to the cells at various times during the infection course, including: 1) during the entire course of infection (FIG. 1A, left panel), 2) during only the first 4 hours of inoculation and then removed (FIG. 1A, middle panel), or 3) only after the 4-hour inoculation (FIG. 1A, right panel). Sixteen hours after initial inoculation, luciferase activity was measured. In a separate experiment, the pooled serum was then left untreated or incubated at 56° C. to inactivate complement in the serum. The serum (or media only) was then added to the Vero cells. WT VSV encoding the green fluorescent protein (GFP) reporter (VSV-GFP) was added to the cells. After 16 hours, the plates were imaged with an imaging cytometer (FIG. 1B, left panel) and the number of GFP-positive cells was quantified (FIG. 1B, right panel).

FIG. 2 shows that human serum inhibits WT VSV in human cell lines. Pooled human serum was mixed with VSV-Fluc virus and overlaid onto human cell lines, e.g., HT1080 human fibrosarcoma cells (FIG. 2, left panel), human embryonic kidney (HEK) 293T cells (FIG. 2, middle panel), or SKOV3.ip1 human ovarian cancer cells (FIG. 2, right panel). The graphs show luciferase activity measured by standard luciferase assay in the three cell lines 16 hours after infection.

FIG. 3 demonstrates that heat-inactivated serum inhibits WT VSV in K562 cells. VSV-GFP was mixed with media, complement-active serum (serum), or heat-inactivated serum (HI serum) and combined with K562 cells. After 24 hours, the plates were imaged with an imaging cytometer and the number of GFP-positive cells in each well was determined.

FIG. 4 shows that heat-inactivated serum inhibits WT VSV in human cell lines. Media alone, complement active human serum pool (serum), or complement inactivated human serum pool (HI serum) was added to human cell lines SKOV3.ip1, HT1080, and K562. VSV-GFP was then added to the cells and the plates were imaged with an imaging cytometer. The number of GFP-positive cells in each well was determined 16 hours after infection.

FIGS. 5A-5B show that serum depleted of lipoproteins does not possess inhibitory activity. Media alone, heat-inactivated serum (serum), human serum albumin (HSA), artificial serum (AF serum), intralipid, or lipoprotein-depleted serum (LD serum) was added to HT1080 or K562 human cell lines. VSV-GFP was then added to the cells. After 16 hours, the plates were imaged with an imaging cytometer and the number of GFP-positive cells in each well was determined (FIG. 5A). Examples of fluorescence microscopy images of the HT1080 cells are shown in FIG. 5B.

FIGS. 6A-6B show that low-density lipoprotein (LDL) and very-low-density lipoprotein (VLDL), but not high-density lipoprotein (HDL), inhibit WT VSV. K562 cells were mixed with VSV-GFP and overlaid into wells of the various test conditions, including media alone (Media; M), human serum pool (S), lipoprotein-depleted human serum pool (D), or media containing various concentrations of purified human LDL, HDL, or VLDL. After 16 hours (FIG. 6A, top panel and FIG. 6B, left panel), cell photos were taken with a fluorescence microscope. After 24 hours, plates were imaged with an imaging cytometer and the number of GFP-positive cells in each well was determined (FIG. 6A, bottom left panel and FIG. 6B, right panel). FIG. 6A, bottom right panel, further displays a graph of luciferase activity (VSV-Fluc) in Vero cells infected 16 hours prior with VSV-Fluc in the presence of various HDL concentrations.

FIG. 7 shows the heat resistance of LDL and VLDL inhibitory activity. Human LDL (FIG. 7, left panel) or VLDL (FIG. 7, right panel) was heat inactivated (HI-LDL or HI-VLDL) or left untreated (LDL or VLDL). K562 cells were mixed with VSV-GFP and overlaid into wells of the various test conditions, including media alone (Media) or heat-inactivated (HI) or untreated LDL or VLDL. Plates were imaged with an imaging cytometer and the number of GFP-positive cells in each well was determined.

FIGS. 8A-8B illustrate mechanisms of VSV activation by serum components. FIG. 8A shows a schematic depiction of pathogen (e.g., VSV) inactivation by IgM plus complement in serum (heat labile). The mechanism of VSV inactivation can be attributed to the binding of natural IgM to the G protein followed by the activation of the complement cascade which leads to coating of the virus with C3b, irreversibly destroying virus infectivity, and subsequent lysis of the virus by the membrane attack complex. This mechanism of virus inactivation is not instantaneous but can reduce the infectious titer of a VSV preparation or of a preparation of lentiviral vectors incorporating the VSV-G protein by up to approximately 10,000-fold over a period of one hour.

FIG. 8B shows a schematic of a mechanism of LDL/VLDL competition with VSV-G for binding to LDLR. ApoB-100 binds to the LDL receptor at the same site that the VSV-G glycoprotein binds. ApoB-100-containing lipoproteins include LDL and VLDL, which compete with VSV-G for binding to LDLR (FIG. 8B).

FIGS. 9A-9B depict designs of retargeted VSV glycoproteins. The VSV-G protein contains a signal peptide (SP) that can be proteolytically cleaved following translation. The targeting molecules can be single chain fragment variable fragment (scFv) antibodies, e.g., raised to the human epidermal growth factor receptor 2 (HER2) or epidermal growth factor receptor (EGFR), natural ligands such as EGFm123 (modified EGF), and/or nanobodies or peptides, and can be appended to the amino-terminus of the G protein, e.g., with or without a flexible linker. Mutations in the G protein can be incorporated at various amino acid positions, e.g., amino acid positions 47 and 354 (K47Q/R354Q) (FIG. 9A). The same targeting molecules can be appended to the N-terminus of alternate Rhabdovirus Flanders G (FLAV-G) with or without a flexible linker (FIG. 9B).

FIGS. 10A-10D illustrate that VSV-G harboring blinding mutations and an anti-HER2 scFv specifically fuses HER2-expressing cell lines. SKOV3.ip1 cells (FIG. 10A), HT1080 cells (FIG. 10B), A549 (human lung adenocarcinoma) cells (FIG. 10C), and BHK-21 (baby hamster kidney) cells (FIG. 10D) stably expressing dual-split protein (DSP) DSP-1 or DSP-2 reporters were co-cultured. The next day, the cells were transfected with plasmids expressing GFP, WT VSV-G or VSV-G harboring an N-terminal anti-HER2 scFv and either two mutations (K47Q/R354Q) or three mutations (K47Q/R354Q/E353A) aimed at ablating the interaction of the glycoprotein with LDLR. After transfection, the cells were treated with pH 5.0 to mediate fusion, or neutral phosphate buffered saline (PBS), then fresh media was added containing the luciferase substrate, EnduRen. Renilla luciferase activity was measured 4 hours after the addition of the substrate.

FIGS. 11A-11B demonstrate that infection of blinded and retargeted VSV-G correlates with HER2 receptor levels. Expression of the HER2 receptor was determined in SKOV3.ip1, Vero, and Hela cells by flow cytometry (FIG. 11A). Cells were infected with VSV-GFP (WT VSV), VSV-αHER2-VSV-G(K47Q/R354Q)-GFP, or VSV-αHER2-VSV-G(K47Q/R354Q/E353A)-GFP. Images were captured by fluorescence microscopy (FIG. 11B).

FIG. 12 demonstrates that soluble αHER2 scFv selectively inhibits HER2 retargeted, LDLR mutant virus infection and spread in SKOV3ip.1 cells. SKOV3ip.1 cells were infected with VSV-GFP, VSV-αHER2-VSV-G (K47Q/R354Q)-GFP, or VSV-αHER2-VSV-G (K47Q/R354Q/E353A)-GFP and treated with media from cultures of cells that had been transfected with plasmids expressing GFP (Mock-sup) or a secreted form of an anti-HER2 or anti-EGFR scFv. Virus infection and spread was monitored by imaging and counting the number of GFP-positive cells using an imaging cytometer.

FIGS. 13A-13B demonstrate that VSV-G can target the EGF or HER2 receptors in the same cell type. SK-BR-3 cells that express both EGFR and HER2 were infected with control VSV-GFP or VSV containing two blinding mutations (K47Q/R354Q) and a targeting molecule to EGFR or HER2 (EGFm123 or an scFv raised to HER2) and then were treated with media (control) or blocking molecules. Twenty hours after infection, the cells were imaged using an imaging cytometer (FIG. 13A) and the number of GFP-positive cells was determined (FIG. 13B)

FIGS. 14A-14B show the specificity of retargeted VSV-αEGFR-G K47QR354Q-GFP in HT1080-EGFR-knockout (KO) and HEK-293T-EGFR-KO cells. HT1080 WT or HT1080-EGFR-KO cells were infected with VSV-GFP (control) or VSV-αEGFR-G K47QR354Q-GFP. The GFP images shown in FIG. 14A (left panel) were taken using fluorescence microscopy and the number of GFP-positive cells was quantified using an imaging cytometer (FIG. 14A, right panel). HEK-293T WT or HEK-293T-EGFR-KO cells were infected with VSV-GFP, or VSV-αEGFR-G K47QR354Q-GFP. The GFP images shown in FIG. 14B were taken using an imaging cytometer. The numbers on the image indicate the number of GFP-positive cells.

FIGS. 15A-15B show specificity of retargeted VSV on the K562 cell panel. K562 parental or EGFR/HER2 receptor expressing cells were infected with VSV-GFP or retargeted VSVs with modified linker sequences (19 amino acid [aa] linker: RAAA(G₄S)₃ (SEQ ID NO: 170); 20 aa linker: KRAAASGGS(G₄S)₂GPK) (SEQ ID NO: 174)). The GFP images shown in FIG. 15A were taken using a fluorescence microscope. The number of GFP-positive cells was quantified using an imaging cytometer (FIG. 15B).

FIGS. 16A-16B show incorporation of targeting molecules EGFm123 or hSCF in the virus particles. Western blotting of EGFm123 displaying virus particles is shown in FIG. 16A. Western blotting of hSCF displaying virus particles is shown in FIG. 16B.

FIGS. 17A-17B illustrate the specificity of retargeted VSV displaying smaller targeting molecules. K562 parental, K562-EGFR, or K562-HER2 cells were infected with VSV-GFP (as a control) or the indicated retargeted VSVs. The GFP images shown in FIG. 17A were taken using a fluorescence microscopy. The number of GFP-positive cells was quantified via an imaging cytometer (FIG. 17B).

FIGS. 18A-18F show VSV-EGFm123 infection in EGFR KO cell lines. For generation of data displayed in FIGS. 18A-18D, cells were infected with VSV-GFP or VSV-EGFm123 virus. GFP and phase microscopy contrast images of HeLa WT or HeLa-EGFR-KO cells were acquired as shown in FIG. 18A. GFP images were taken with an imaging cytometer (FIG. 18B) and the infection level was quantified as the number of GFP-positive cells for the HeLa cell panel (FIG. 18C) or HT1080 cell panel (FIG. 18D). The ability of VSV-EGFm123 to bind to the intended receptor was determined using HeLa or HT1080 WT or EGFR-KO cells (FIGS. 18E-18F). 1×10⁶ cells were incubated with VSV-GFP or VSV-EGFm123 virus. The cells were fixed, followed by staining with a PE-conjugated VSV-G antibody. The cells were analyzed by flow cytometry (FIGS. 18E-18F).

FIGS. 19A-19B demonstrate that the VSV-G-QQ-EGFm123 virus is resistant to inhibition by LDL. VSV-GFP (VSV-G-wt), or VSV-GFP with G containing the K47Q/R354Q mutation and appended to EGFm123 (EGFR retargeted virus), was incubated with media alone, human pooled serum (complement deficient lot), or pooled lipoprotein-depleted human serum. The mixtures were overlaid onto plated HEK-293T. After 24 hours, plates were imaged with an imaging cytometer and the number of GFP-positive cells per well was determined (FIG. 19A). VSV-GFP (VSV-G-WT), or VSV-GFP with G containing the K47Q/R354Q mutation and appended to EGFm123 (EGFm123-K47Q/R354Q), was mixed with K562-EGFR cells and added to wells containing media alone (control) or increasing concentrations of purified human LDL. After 24 hours, plates were imaged with an imaging cytometer and the number of GFP-positive cells per well was determined (FIG. 19B).

FIG. 20 demonstrates that the VSV-G-QQ-EGFm123 virus is resistant to inhibition by VLDL. VSV-GFP (WT-G) or VSV-GFP with G containing the K47Q/R354Q mutation and appended to EGFm123 (EGFm123-K47Q/R354Q), was mixed with K562-EGFR cells and added to wells containing media alone or purified human HDL, LDL, or VLDL at the indicated concentrations. After 24 hours, plates were imaged with an inverted fluorescence microscope.

FIG. 21 shows that binding of WT VSV, but not EGFR retargeted VSV, is reduced by LDL. VSV-GFP (VSV-G-WT), or VSV-GFP with G containing the K47Q/R354Q mutation and appended to EGFm123 (VSV-MC11-EGFm123-VSV-G(K47Q/R354Q)-GFP), was mixed with media alone, pooled human serum (serum) or LDL and overlaid onto HT1080 cells. RNA was then extracted from the cells and samples were subjected to quantitative reverse transcription polymerase chain reaction (qRT-PCR) using primers specific for the VSV genome (IDT). Data represent the number of VSV genome copies relative to the number of copies from the media only control for the virus.

FIG. 22 illustrates the sensitivity of retargeted VSV-Gs to human serum and LDL. VSV-GFP (VSV-G-WT), or VSV-GFP with G containing the K47Q/R354Q mutations and appended to either human stem cell factor (hSCF) (VSV-GFP (hSCF-G-K47Q/R354Q)) or HER2 scFv (VSV-GFP (G-HER2)), were mixed with various cells from a K562 cells panel, including K562 parental, K562-HER2, or K562-cKit. Mixes were added to wells containing media alone, human pooled serum (serum), human pooled lipoprotein-depleted serum (LD Serum), media containing LDL (+LDL), or human pooled lipoprotein-depleted serum with LDL (LD Serum+LDL). Fluorescence microscopy photos were acquired after 24 hours.

FIG. 23 shows the sensitivity of retargeted VSV-Gs to human serum and LDL in PC3 cells. VSV-GFP (WT-G), or VSV-GFP with G containing the K47Q/R354Q mutations and appended to either EGFm123 (G-QQ-EGFm123), hSCF (G-QQ-SCF), or HER2 scFv (G-QQ-HER2), were mixed with media alone, fresh (complement active) human pooled serum (serum), heat-inactivated pooled human serum (HI-serum), or human pooled lipoprotein-depleted serum with LDL (+LDL). Mixes were overlaid onto PC3 prostate cancer cells. After 24 hours (WT) or 42 hours (retargeted viruses), cells were imaged using an imaging cytometer.

FIG. 24 shows cytoplasmic tail truncation of FLAV-G enhances the fusion activity of FLAV-G. A panel of FLAV-G constructs comprising an anti-EGFR scFv with successively shorter cytoplasmic tails was generated. The fusion activity shown in the graph was determined by DSP cell-cell fusion activity in SKOV3.ip1 cells.

FIGS. 25A-25C illustrate improved VSV targeting to the HER2 receptor in PC3 cells. A schematic of a FLAV-G construct design with a 23 aa linker and VSV-G cytoplasmic tail and a construct with a 19 aa linker lacking the VSV-G cytoplasmic tail is shown in FIG. 25A. Two scFvs targeting HER2 and one targeting EGFR were cloned into the 19 aa linker construct. For generation of data displayed in FIGS. 25B-25C, the specificity of two HER2-targeted viruses was assessed on a panel of PC3 cells including parental PC3, PC3-EGFR, and PC3-HER2. Fluorescence microscopy images of infected cells using a microscope are shown in FIG. 25B. The number of infected cells in each condition was determined using an imaging cytometer (FIG. 25C).

FIGS. 26A-26C show FLAV-G targeted to insulin-like growth factor 1 (IGF1) receptor. For generation of data displayed in FIGS. 26A-26B, MCF7 breast cancer cells were infected with VSV-GFP (WT) or VSV containing a Flanders virus glycoprotein (FLAV-GA30) that was free from any targeting molecule (no Targeting) or targeted to the IGF1 receptor (IGF1R) with insulin like growth factor 1 (IGF1). Infected cells were imaged by fluorescence microscopy (FIG. 26A, top panel) and phase contrast microscopy (FIG. 26A, bottom panel). The infected cultures were also imaged using an imaging cytometer and the number of GFP-positive cells was quantified (FIG. 26B). Flow cytometry was used to determine the expression of the IGF1 receptor in cells that had been fluorescently labeled anti-IGF1R antibody or an isotype control (FIG. 26C).

FIGS. 27A-27B show specificity of FLAV-G displaying modified epidermal growth factor (EGF). K562 cells stably transduced with the indicated receptors were infected with VSV-GFP (WT) or VSV containing a Flanders virus glycoprotein (FLAV-GA30) displayingEGFm123. Infected cells were imaged by fluorescence microscopy (FIG. 27A, top panels) and phase contrast microscopy (FIG. 27A, bottom panels). The infected cultures were also imaged using a cytometer and the number of GFP-positive cells was determined (FIG. 27B).

FIGS. 28A-28C show the design of retargeted VSV-G for lentivirus production. A schematic of the constructs generated is shown in FIG. 28A. The VSV-G protein contained a signal peptide (SP) that could be proteolytically cleaved following translation. Example targeting molecules include an scFv raised to HER2 or EGFR, natural ligands, e.g., EGFm123 (modified EGF) or hSCF, or nanobody, e.g., Nb 7D12 against EGF receptor, which were appended to the amino-terminus of the G protein, with or without a flexible linker. A construct without a targeting molecule was generated as a control. Blinding mutations in VSV-G were incorporated at K47 and R354 (FIG. 28A). Figure discloses SEQ ID NO: 35, 35, 35, and 236, respectively, in order of appearance. Lentivirus production in HEK-293T cells is schematized in FIG. 28B. Western blotting of the lentivirus particles is shown in FIG. 28C.

FIGS. 29A-29B illustrate the validation of retargeted lentiviruses on the receptor positive cell lines. Lentiviruses pseudotyped with VSV-G-WT or VSV-G harboring K47Q/R354Q (G-QQ) mutations and appended to EGFR scFv or EGFR-E11 scFv or EGFm123 ligand were transduced into parental Jurkat or K562 cells, or modified version of the cells that over-expressed EGFR. Fluorescence microscopy images were taken at 48 hours post transfection (hpt) for the Jurkat cells (FIG. 29A). K562 cells were transduced with viral supernatant and the fluorescence microscopy images were taken at 24 hpt (FIG. 29B).

FIGS. 30A-30B demonstrate that an EGFR-retargeted lentiviral vector is less sensitive to serum and LDL inhibition. A set volume of a GFP-expressing lentiviral vector pseudotyped with WT VSV-G (WT-G) or VSV-G harboring the K47Q/R354Q mutation and appended to EGFm123 (EGFm123 K47Q/R354Q) was used to transduce either K562 parental cells or modified EGFR-overexpressing K562 cells (K562-EGFR). Transductions were carried out in the presence of media alone, pooled human serum (complement deficient), pooled lipoprotein-depleted human serum, or pooled lipoprotein-depleted human serum spiked with 150 mg/dL LDL. After 40 hours, cells were imaged by both fluorescence microscopy (FIG. 30A) and imaging cytometer. The number of GFP-positive cells per well was determined using the cytometer software (FIG. 30B).

FIGS. 31A-31D illustrate that VSV-GH8Q/K47Q/Y209Q/R354Q mutations enhanced the specificity of retargeted VSV displaying EGFm123. Construct designs containing the LDLR blinding mutations in the VSV-G are shown in FIG. 31A. For data generated for FIGS. 31B-31C, K562 parental cells or K562-EGFR cells were infected with the VSV-EGFm123 viruses and GFP images were acquired with a fluorescence microscope (FIG. 31A, left panel). Quantification of GFP-positive cells by an imaging cytometer is shown in FIG. 31A, right panel. GFP images captured by an imaging cytometer are shown in FIG. 31D. These results indicated that combining the Y209Q mutation with VSV-G K47Q/R354Q enhanced the specificity of retargeted VSV displaying EGFm123, while combining the H8Q mutation with VSV-G K47Q/R354Q had minimal or no effect on the enhancement of retargeting specificity.

FIGS. 32A-32D demonstrate that deletion of the K47 residue in the VSV-G ablates the VSV tropism and redirects VSV to EGF receptor (EGFR) positive cells. K562 parental cell or K562-EGFR was infected with the VSV-GFP or EGFm123 displaying G-WT or G-ΔK47 VSVs and fluorescence microscopy images were acquired (FIG. 32A, left panel). Quantification of GFP-positive cells via imaging cytometer is shown in FIG. 32A, right panel. Examples of GFP images captured by an imaging cytometer are shown in FIG. 32B. Additional constructs comprising deletion of H8, K47, Y209 and/or R354 residues contemplated for impairment of LDLR tropism of VSV is shown in FIG. 32C. Further examples of constructs comprising double residue deletion mutations is shown in FIG. 32D.

FIG. 33 shows sequence confirmation of VSV-G-ΔK47 residue in VSV1-409-0. The virus sequence shows a point mutation at F405I in the VSV-G. Figure discloses SEQ ID NO: 213-226, respectively, in order of appearance.

FIGS. 34A-34B show targeting with alternate Rhabdoviral G proteins. FIG. 34A depicts selection of nine glycoproteins from nine different rhabdovirus genera (arrows). FLAV-G was selected as a lead for targeting. VSVs containing a GFP reporter gene and FLAV GA30 with scFvs targeting EGFR or HER2 were used to infect a panel of K562 cells that stably expressed (or not) EGFR or HER2. The cells were imaged using a fluorescence microscope (FIG. 34B).

FIG. 35 depicts screening of additional alternate G proteins. Twenty additional viral glycoproteins were selected for screening to identify glycoproteins that can be retargeted to receptors of interest. A description of virus species in the vesiculovirus genus of Rhabdoviridae is shown in the table (FIG. 35, left panel). The percent amino acid sequence identity to vesicular stomatitis Indiana virus (VSIV) (see, e.g., SEQ ID NO: 8) was computed using Clustal Omega with or without the signal peptide sequence included. Additional glycoproteins from the Ledantevirus, Hapavirus and Ephemerovirus genera were selected based on favorable properties of Fukuoka (Ledantevirus), Flanders (Hapavirus) and Bovine ephemeral fever (Ephemerovirus) virus glycoproteins in the first phase of screening (FIG. 35, right panel).

FIG. 36 shows screening of additional non-VSV rhabdoviral G proteins using an alternate format. Twenty additional viral glycoproteins were selected for screening to identify glycoproteins that can be retargeted to receptors of interest and may be resistant to complement inactivation (FIG. 36, left panel). Virus species in the vesiculovirus genus of Rhabdoviridae were selected based on having a percent amino acid sequence identity less than 70% to vesicular stomatitis Indiana virus. Additional glycoproteins from the Ledantevirus, Hapavirus and Ephemerovirus genera were selected based on favorable properties of Fukuoka (Ledantevirus), Flanders (Hapavirus) and Bovine ephemeral fever (Ephemerovirus) virus glycoproteins in the first phase of screening. For each glycoprotein, the signal peptide sequence was predicted using SignalP 6.0 and a modified epidermal growth factor (EGFm123) was inserted immediately following the signal peptide sequence. These modified glycoprotein genes were synthesized and subcloned into a protein expression vector (pCG) (FIG. 36, right panel).

FIG. 37 shows functional screening of alternate EGF-displaying rhabdoviral G proteins for EGFR targeting. A schematic of the DSP cell-cell fusion assay described herein is shown in FIG. 37, left panel. Measurement of fusion activity of the various glycoproteins described herein in SKOV3.ip1 cells and in EGFR KO SKOV3.ip1 cells is shown in FIG. 37, middle panel. A bar graph displaying cell surface expression of EGFm123-G proteins measured by median fluorescent intensity across EGFm123-G protein plasmids (top) and a western blot analysis of EGFm123 levels for the EGFm123-G protein plasmids (bottom) is shown in FIG. 37, right panel.

FIG. 38 illustrates pseudotyping of lentiviruses with targeted G proteins. Lentiviruses pseudotyped with a panel of targeted rhabdovirus glycoproteins (or non-targeted VSV G WT and EGFR-targeted FLAV-GA30 as controls) were generated by expressing the glycoprotein vector (pCG), packaging plasmid (p8.91) and the genome vector expressing GFP (pLV-SFFV-GFP) in HEK 293T cells (FIG. 38, left panel). Lentivirus supernatant was then added to monolayers of SKOV3.ip1 or SKOV3.ip1 EGFR KO cells. The number of GFP-positive cells was determined using an imaging cytometer (FIG. 38, right panel).

FIG. 39 shows a description of various features of a dual-split protein (DSP) screen described herein and a lentivirus screen described herein.

FIG. 40 illustrates an example experimental time course of VSV-G EGFR scFv transfection screen described herein.

FIG. 41 shows an example of a protocol for an EGFR scFv DSP transfection assay screen described herein.

FIG. 42 shows a flowchart of an example analysis for the EGFR scFv transfection screen.

FIG. 43 demonstrates that the EGFR scFv screen in SKOV3ip.1 DSP screen identified several scFv sequences with improved fusion.

FIG. 44 shows αEGFR-VSV-G indicates moderate preference towards scFv orientation.

FIG. 45 is a schematic of orientation preference indicative of increased function when the scFv is on the N-terminal end of ectodomain.

FIG. 46 shows an example αEGFR lentivirus production protocol described herein.

FIG. 47 shows a description of αEGFR titration by qPCR.

FIG. 48 shows a flowchart of an example lentivirus screen described herein.

FIG. 49 illustrates several αEGFR scFv-VSV-G(QQ) displaying lentiviruses demonstrate enhanced transduction efficiency in the presence of serum.

FIG. 50 shows a description of transduction efficacy of example αEGFR scFv candidates.

FIG. 51 illustrates comparison of DSP and lentivirus screens for the αEGFR-VSV G constructs including comparison of scFv sequences using Multiple Sequence Comparison by Log-Expectation (MUSCLE) and graphical representation in a phylogenetic tree aligned using Jalvuew software and BLOSUM62 nearest neighbor algorithm.

FIG. 52 depicts VSV construct designs comprising various linker sequences. Retargeted VSV comprising a 20 aa linker between the EGFR scFv and VSV-G which was proteolytically cleaved is included. Different types of linker sequences with variable lengths were cloned between the EGFR scFv and VSV-G. EIK, terminal amino acid sequence of scFv; KFT, starting amino acid sequence of VSV-G. The linker sequence shown in red text. F, flexible linker; R, rigid linker; F-m, medium flexible linker; F-el, flexible elastin-like linker; IgG4 h, IgG4 hinge. The VSV-G contains the K47Q and R354Q blinding mutations to LDLR. Figure discloses SEQ ID NO: 171-172, 36-39, 3, 173, and 40-41, respectively, in order of appearance.

FIG. 53 shows rescue of EGFR-targeted VSV with alternate linker sequences. pVSV-MC11-EGFRscFv-VSV-G-GFP plasmids with alternate linker sequences were rescued in SKOV3.ip1 cells using a vaccinia-based rescue system. The virus supernatants were collected and filtered (p0 supernatant). The p0 supernatant was then transferred to the monolayer Vero-EGFR cells and the GFP fluorescence microscopy images shown in the figure were acquired.

FIGS. 54A-54C show characterization of EGFR targeted VSVs. Amplification stage, virus titers, and sequencing results are described in the table displayed in FIG. 54A. Western blotting of virions is depicted in FIG. 54B. Bands show EGFR scFv intact with the G and the proteolytically cleaved G. Specificity of the EGFR targeted VSVs is illustrated in FIG. 54C. Photomicrographs show K562 parental or K562-EGFR following infection with EGFR targeted VSVs along with VSV-GFP and fluorescence micrographs were acquired.

FIG. 55 demonstrates that the 18aaL(F), 15aaL(R), and 16aaL(R) linkers exhibit proteolytic cleavage. Nine different linkers attached to EGFR scFv were selected for determination of linkers providing specific targeting to EGFR (FIG. 55, left panel). Linker sequences were produced in the VSV backbone and then subcloned into the pCG vector backbone for use in lentivirus production. Constructs were transfected into HEK293T cells and cell lysates were collected and run on an SDS-PAGE gel to analyze protein expression (FIG. 55, right panel). Figure discloses SEQ ID NO: 166-167, 26-30, 168, and 31-32, respectively, in order of appearance.

FIG. 56 illustrates that the 18aaL(F), 15aaL(R), 16aaL(R) linkers exhibit fusogenic capability. Fusion activity of the nine constructs that contain EGFR scFv with variable linkers was measured in a mixed population of A549-EGFR expressing DSP1-7 and DSP8-11 cells. Half contained a dual-split protein (DSP) reporter gene that has a split GFP and half that contain Renilla luciferase.

FIGS. 57A-57B illustrates that the 18aaL(F), 15aaL(R), and 16aaL(R) linkers exhibit proteolytic cleavage in lentivirus production cell lysates and the lentivirus particle itself. FIG. 57A depicts a schematic representation of EGFR scFv with variable linkers virus production. Lentivirus production plasmids were transfected into HEK 293T cells to produce lentiviruses. Western blots of cell lysates from the cells that were used to produce the lentiviruses were run to detect EGFR scFv-linker-VSV-GQQ (full length) or the cleaved VSV-GQQ (FIG. 57B, left panel). Supernatant that contained the virus particles was collected and spun down in a microcentrifuge. The supernatant was aspirated, cell pellet was lysed, reduced at 95° C. for 5 minutes and then run on an SDS-PAGE gel to detect EGFR scFv-linker-VSV-GQQ (full length) or the cleaved VSV-GQQ (FIG. 57B, middle panel). The titer of each virus as determined by p24 enzyme-linked immunosorbent assay (ELISA) is also included.

FIGS. 58A-58B illustrates that the 18aaL(F), 15aaL(R), and 16aaL(R) linkers that exhibit proteolytic cleavage demonstrate increased specificity for targeting K562-EGFR expressing cells. K562 parental and K562-EGFR expressing cells were transduced with each lentivirus and GFP fluorescence microscopy and bright field (BF) images were captured post transduction to determine specificity of each retargeted lentivirus with a different linker (FIG. 58A). A graph of the number of GFP positive cells quantified in K562 and K562-EGFR cells transduced with each lentivirus is depicted in FIG. 58B.

FIG. 59 shows targeting constructs used to evaluate virus specificity in presence or absence of serum. Displayed ligands included human EGF, human stem cell factor (hSCF) and scFvs against EGFR or Her2. The hSCF ligand was displayed on a blinded (VSV-G-QQ) G protein that had poor interaction with LDLR, or an unblinded (VSV-G-WT) G protein.

FIG. 60 depicts fluorescence microscope images of K562 cells, parental or stably expressing HER2, cKit (SCF receptor), or EGFR, infected in the absence (left panel) or presence (right panel) of 25% heat inactivated human serum, with recombinant VSVs incorporating the G proteins shown in FIG. 59. Infected cell monolayers were imaged under blue light 42 hours post infection (hpi) (24 hpi for VSV-GFP with unmodified G protein). The left panel shows that ligand-displaying viruses specifically infected only those target cells that bore the cognate receptor for their displayed ligand. This was apparent even for the SCF-displaying virus wherein the G protein had not been mutated to ablate LDLR tropism, indicating that the displayed SCF domain could sterically interfere with the G protein-LDLR interaction. The right panel shows that, in the presence of 25% heat inactivated human serum, entry of the virus bearing a wild type G protein was blocked in all K562 clones, whereas the entry of targeted viruses via alternate (i.e., non-LDLR) receptors was not blocked and may even be enhanced in certain cases.

FIG. 61 depicts proof-of-concept data supporting retargeting of G-pseudotyped lentiviral vectors via displayed domains including EGFm123 and anti-epidermal growth factor receptor (EGFR) scFv (E11).

FIG. 62 demonstrates that increasing the amount of VSV G-QQ DNA increases the amount of VSV G-QQ protein proportionally. The first part of the figure shows a pictorial version of the lentivirus production protocol along with a table that has the amount of envelope glycoprotein DNA used. The bottom right contains a western blot of VSV G (from the uncleaved chimera and VSV G-QQ). Rabbit anti-VSV G [8G5F11] antibody was used to probe for protein in the virion pellet.

FIGS. 63A-63B show that increasing the ratio of VSV G-QQ to the non-cleaving EGFR scFv-17aaL(F)-VSV G-QQ improves targeting specificity. For FIG. 63A, top panels depict transductions of K562 and K562-EGFR expressing cells with lentiviruses that have a mixed ratio of EGFR scFv-17aaL(F)-VSV G-QQ and VSV G-QQ. Viruses were transduced at an multiplicity of infection (MOI)=5. Brightfield (FIG. 63A, bottom panels) and GFP (FIG. 63A, top panels) expression of each lentivirus was imaged at 72 hours post transduction (hpt). The amount of DNA that corresponds to each ratio listed at the top of the panel set is indicated in the table below all the panels. The first number indicates the amount of VSV G-QQ DNA used and the second number represents the amount of EGFR scFv-17aaL(F)-VSV G-QQ DNA used. Decreasing the amount of EGFR scFv-17aaL(F)-VSV G-QQ DNA and increasing the amount of VSV G-QQ DNA yields improved targeting specificity. Celigo quantifications of the number of GFP positive cells for each mixed ratio virus is shown in FIG. 63B.

FIG. 64 illustrates a potential model of a mixed trimer for a non-cleaving retargeted VSV G and a blinded VSV G. The model on the left depicts a homo-trimeric VSV G. Each monomer includes a EGFR scFv retargeting domain, linker, and the blinded VSV G (mutations at K47Q and R354Q-QQ). For the lentiviruses that did not specifically target or cleave initially, mixing non-cleaving EGFR scFv retargeted monomers with the blinded VSV G-QQ, may yield a hetero-trimer that allows for targeting specificity.

FIGS. 65A-65C show in vivo targeting of an EGFR+(epidermal growth factor receptor positive) tumor by VSV displaying EGF (epidermal growth factor), administered intravenously (i.v.) in a SCID (severe combined immunodeficiency disease) mouse model. For the study design (FIG. 65A), female CB17 SCID mice were implanted subcutaneously with murine myeloma 5TGM1 cells expressing human EGF receptor (hEGFR). When the mean tumor volumes were around 150 mm³, mice were randomized and separated into groups. Mice were treated intravenously with either saline, or 1×10⁸ TCID₅₀ VSV-M-RFP-GFP or 1×10⁸ TCID₅₀ VSV-M-GqqEGFm123-GFP virus. Mice were observed for adverse clinical signs, body weight and tumor growth until D40 (day 40) post treatment. A 5TGM1-hEGFR+tumor growth profile is shown in FIG. 65B. Mice treated with VSV-M-RFP-GFP developed adverse clinical signs and they were either found dead or sacrificed. By D21 post infection, there were no surviving mice (FIGS. 65B-65C). In contrast to mice treated with VSV-M-RFP-GFP, VSV-M-GqqEGFm123-GFP virus completely arrested tumor growth in all 6 treated mice. No mice developed adverse clinical signs (FIGS. 65B-65C).

FIGS. 66A-66D illustrate optimization of the cytoplasmic tail of KRV-G. Fusion activity of KRV G cytoplasmic tail truncation mutants. SKOV3.ip1 cells stably transduced with either the N-terminal or C-terminal half of a dual split protein GFP-Renilla luciferase reporter were seeded in equal proportions. The following day, plasmids that express VSV-G WT or EGFm123-modified (at the N-terminus), FLAV-G with the C-terminal 30 amino acids removed (Δ30) or KRV-G with cytoplasmic tail truncations (e.g., 10 amino acids removed (Δ10), 20 amino acids removed (Δ20), or 30 amino acids removed (Δ30)) were transfected into the cells. The day after transfection, the cells were treated with pH 5.0 or pH 7.4 (as a control) phosphate buffered saline (PBS) for 2 minutes, and then the saline was replaced with fresh media containing EnduRen substrate. The resulting luminescence was measured 2 hours later (FIG. 66A). GFP-encoding VSVs with KRV-G or KRV-G with the C-terminal 10 amino acids removed (Δ10) and N-terminally modified with an EGFm123 molecule were rescued and amplified. The infectious titers of the resultant viruses were determined by TCID₅₀ assay (FIG. 66B). The specificity of full-length or Δ10 KRV-G was determined by monitoring the course of infection of recombinant VSV bearing these EGFm123-tagged glycoproteins and a green fluorescent protein (GFP) (FIGS. 66C-66D). The recombinant viruses were used to infect wild-type (WT) or EGF receptor (EGFR)-knockout (KO) versions of SKOV3.ip1 (FIG. 66C) or HEK293T (FIG. 66D) cells at a MOI of 1 and the number of infected cells expressing virus-encoded GFP were measured using a Celigo image cytometer at 1 day post infection.

FIGS. 67A-67B demonstrate epidermal growth factor receptor (EGFR)-targeted KRV-G and KEUV-G are more resistant to serum inhibitory factors and have comparable specificity to blinded VSV-G. GFP-encoding VSVs with WT VSV glycoprotein (VSV-G), or with one of the following modified glycoproteins replacing the native VSV-G, were generated: VSV-G containing LDLR-receptor blinding glutamine substitution at K47 and R354 (QQ) or K47, Y209, and R354 (QQQ), Kumasi rhabdovirus G (KRV-G), KRV-G with the C-terminal 10 amino acids removed (Δ10), Keuraliba virus G (KEUV-G), Perinet virus G (PERV-G), Piry virus G (PIRYV-G), Fukuoka virus G (FUKV-G) or Curionopolis virus G (CURV-G). The indicated viruses contained a modified epidermal growth factor (EGF) molecule appended to the N-terminus of the encoded glycoprotein. The sensitivity of the modified VSVs to complement-active serum was determined by incubating 1×10⁶ infectious units of VSV in OptiMEM media alone, OptiMEM with 50% heat inactivated human serum or OptiMEM with 50% complement active serum for 1 hour at 37° C. The incubated virus was serially diluted and used to inoculate Vero cells stably expressing human EGFR in a standard TCID₅₀ titration assay. Titer plates were scored at 3 days after inoculation (FIG. 67A). The specificity of VSVs retargeted to the EGFR using various rhabdovirus glycoproteins was determined by monitoring the course of infection of recombinant VSV encoding a GFP gene over 3 days. HT1080 cells (WT) or HT0180 cells in which the EGFR gene was knocked out (KO) were infected with the indicated VSVs at a MOI of 1, and the number of infected cells expressing virus-encoded GFP were measured using a Celigo image cytometer at 1, 2 and 3 days post infection (FIG. 67B).

FIGS. 68A-68B illustrate that the Kumasi rhabdovirus glycoprotein (KRV-G) can be retargeted to EGFR. The course of infection of recombinant VSV encoding a GFP gene was monitored over the course of 3 days. For the viruses represented in FIG. 68A, the glycoprotein gene was replaced by the KRV-G in which the C-terminal 10 amino acids were removed (Δ10). Where indicated, a targeting moiety was added (either modified epidermal growth factor (EGF) or an scFv targeting EGF receptor (EGFR)) to the N-terminus of the KRV-G. SKOV3.ip1 cells (WT) or SKOV3.ip1 cells in which the EGFR gene was knocked out (KO) were infected with the indicated VSVs at an MOI of 1, and the number of infected cells expressing virus-encoded GFP were measured using a Celigo image cytometer at 1, 2 and 3 days post infection. For the viruses represented in FIG. 68B, the VSV-G gene was mutated to contain either two (QQ) or three (QQQ) mutations to ablate LDLR binding, or was replaced by the glycoprotein from Kumasi rhabdovirus (KRV-G) in which the C-terminal 10 amino acids were removed (Δ10). Where indicated, a targeting moiety was added (either modified epidermal growth factor (EGF), an scFv targeting EGF receptor (EGFR), or an scFv targeting Her2 to the N-terminus of the KRV-G or VSV-G. SKOV3.ip1 cells (WT) or SKOV3.ip1 cells in which the EGFR gene was knocked out (KO) were infected with the indicated KRVs or VSVs at an MOI of 1, and the number of infected cells expressing virus-encoded GFP were measured using a Celigo image cytometer at 1, 2 and 3 days post infection.

FIG. 69 shows examples of designs of retargeted mutant VSV-G constructs. The VSV-G protein contains a signal peptide (SP) that is proteolytically cleaved following translation. The targeting molecule is a natural ligand such as, but not limited to, EGFm123 (modified EGF), which can be appended to the amino-terminus of the G protein without a flexible linker. The LDLR binding residues (H8, K47, R354 and Y209) have been deleted in the G protein either in single, double, triple or quadruple combinations.

FIG. 70 depicts protein expression of VSV-G deletion mutant constructs. The plasmid DNA constructs were transfected along with the lentiviral transfer and packaging constructs to produce the lentivirus in the HEK-293T cells. For comparison, substitution mutants, triple or quadruple, combined with G-QQ (K47QR354Q), were included in this experiment. After the collection of lentivirus supernatants, the cell lysates were collected at 72 hours post-transfection, followed by western blotting with anti-VSV-G and anti-GAPDH (glyceraldehyde 3-phosphate dehydrogenase) antibodies.

FIG. 71 demonstrates incorporation of VSV-G deletion mutants into lentivirus particles. Lentivirus supernatants collected at 72 hours post-transfection were titered by a p24 ELISA method. A total of 5×10⁵ physical particles were lysed and loaded onto SDS-PAGE, followed by Western blotting with anti-VSV-G and anti-p24 antibodies.

FIGS. 72A-72D depicts screening of deletion mutants ablating VSV-G tropism. Lentivirus transduction on the K562 cell panel is shown in FIGS. 72A-72B. K562 parental or K562-EGFR cells were transduced with the indicated retargeted lentiviruses displaying EGFm123 at a MOI=20. Lentivirus pseudotyped with WT-G was used as a control. Celigo images (FIGS. 72A-72B) and Nikon images (FIG. 72C) were taken at 72 hours post-transduction and transduced GFP positive cells were quantified (FIG. 72D).

### DETAILED DESCRIPTION OF THE INVENTION

The glycoprotein of rhabdoviruses is the sole viral protein found on the virion surface and mediates binding of the virus particle to a cellular receptor and subsequent viral entry into the cell (infection). The glycoprotein (G) of vesicular stomatitis virus (VSV) mediates infection into a wide variety of cell types from a wide range of species through interaction with the relatively ubiquitous low density lipoprotein receptor (LDLR) and related receptor family members (Finkelshtein et al., 2013; Nikolic et al., 2018). VSV-G can be used to pseudotype lentiviruses for multiple therapeutic applications, including, for example, chimeric antigen receptor (CAR)-T cells, and several VSV-G-pseudotyped lentiviruses are in clinical trials (Munis et al., 2020).

CAR-T cells can be used for treatment of hematological cancers. Yet, logistical challenges related to ex vivo manufacturing of CAR-T cells can be a limiting factor in more widespread clinical adoption of the therapy. Currently, CAR-T cells are engineered ex vivo primarily using VSV-G-pseudotyped lentiviral vectors that deliver the CAR to target T-cells collected from the patient. The modified CAR-T cells are subsequently expanded ex vivo and infused back into the patient. In vivo CAR-T therapy involves delivering the CAR-encoding viral vector directly to the patient to achieve modification and proliferation of the T-cells within the patient, effectively overcoming many of the logistical challenges currently associated with CAR-T therapy. However, in vivo CAR-T therapy requires effective targeting of CAR-encoding viral vectors to target T-cells while limiting off target effects. Because most candidate lentiviral vector therapies used with CAR-T cells are currently pseudotyped with VSV-G, solutions for effectively targeting VSV-G have applications for both oncolytic VSV therapies as well as CAR-T cells.

In addition to appropriate targeting of VSV-G-based therapies, effective therapies must also circumvent natural barriers to infection long enough to reach target cells/tissues once delivered into the body. Oncolytic VSVs, as well as VSV-G-pseudotyped lentiviral vector therapies, can be inactivated by the complement system (DePolo et al., 2000; Mills and Cooper, 1978). Manufacture of VSVs or VSV-pseudotyped lentiviral vectors in cells expressing high levels of CD55 can significantly enhance virus/vector resistance to complement inactivation (Schauber-Plewa, C. et al., 2005; Johnson et al. 2012). However, as described in this disclosure, an additional natural barrier has been identified within human serum (blood) that blocks binding of oncolytic VSV or VSV-G-pseudotyped lentiviral to cells. This barrier is ApoB-100-containing lipoproteins, which outcompete VSV-G for binding to the low-density lipoprotein receptor (LDLR) and thereby prevent VSV-G-based therapies from being efficiently delivered to cells. Effective therapy delivery, therefore, will also require overcoming or bypassing competition with ApoB-100-containing lipoproteins.

The recombinant fusogenic proteins of the present disclosure have the advantage of both greatly enhancing targeting towards cellular receptors that are highly expressed in a variety of cancers while also circumventing inhibition by LDL/VLDL during therapy delivery. The extensive data with different linker combinations highlights the importance of linker sequence and length in determining the function of retargeted G proteins and highlight the need for a proper linker to result in functional G-based therapies.

This disclosure demonstrates retargeting of exemplary VSV-Gs using LDLR-blinding mutations (e.g., corresponding to positions H8, K47, Y209, and/or R354 in SEQ ID NO: 8) combined with scFv antibodies to EGFR or HER2 or nanobodies, as well as specific retargeting in the absence of blinding mutations using VSV-G with a modified EGF (EGFm123) or stem cell factor (SCF). A distantly related rhabdovirus glycoprotein (FLAV-G) could be retargeted to EGFR and HER2, with HER2 retargeting requiring optimization of linker sequence/length. This disclosure could be expanded to include additional glycoproteins from the rhabdovirus family in place of VSV or FLAV-G, such as those detailed below. Receptors other than EGFR and HER2 could be targeted in a similar way with e.g., scFv molecules or other ligands. While the data presented here describe the use of scFvs, nanobodies, and natural receptor ligands for targeting G, other targeting molecules, including but not limited to, scFvs, affibodies, darpins, peptides, nanobodies, and natural or modified natural receptor ligands, could also be used. The data presented here primarily show VSV targeting using replication-competent virus, but a VSV platform in which the retargeted glycoprotein is not encoded in the genome, but rather provided in trans, could also be used in cases where a replicating vector is not desired. Furthermore, the VSV glycoprotein can be used to pseudotype other viruses including lentiviral vectors for gene therapy applications. Retargeted rhabdovirus glycoproteins would be highly desirable in those applications to tailor the targeting of the therapy to cells of interest. To that end, the present disclosure illustrates that strategies used to specifically retarget VSV-G in the context of replicating VSV are transferable to pseudotyped lentiviral vectors.

Importantly, data described herein show that not only did targeting of the G proteins accomplish specific targeting to cells of interest, but also that targeting circumvented a previously unrecognized natural barrier to infection: competition of VSV-G with LDL/VLDL for LDLR binding. Since LDL/VLDL exhibited a dose-dependent inhibitory response, an alternative way to counter competition could be pre-treatment of patients with LDL/VLDL-lowering medications to limit the amount of these competitive molecules in the bloodstream at the time of treatment. However, such alternative would require delay of treatments long enough for blood LDL/VLDL levels to drop to levels that would not cause inhibition of the delivered therapies.

Further details of the compositions and methods of present disclosure are described in the various exemplary embodiments below.

### Definitions

Unless defined otherwise, all technical and scientific terms used herein have the same meaning as commonly understood by one of ordinary skill in the art to which this disclosure belongs.

Singular forms “a”, “an”, and “the” include plural references unless the context clearly dictates otherwise. Thus, for example, a reference to “a method” includes one or more methods, and/or steps of the type described herein and/or which will become apparent to those persons skilled in the art upon reading this disclosure.

The term “about” or “approximately” includes being within a statistically meaningful range of a value. Such a range can be within an order of magnitude, preferably within 50%, more preferably within 20%, still more preferably within 10%, and even more preferably within 5% of a given value or range. The allowable variation encompassed by the term “about” or “approximately” depends on the particular system under study, and can be readily appreciated by one of ordinary skill in the art.

The term “antigen” refers to any agent (e.g., protein, peptide, polysaccharide, glycoprotein, glycolipid, nucleic acid, portions thereof, or combinations thereof) that, when introduced into a host, animal or human, having an immune system (directly or upon expression as in, e.g., DNA vaccines), is recognized by the immune system of the host and is capable of eliciting, or does elicit, an immune response.

The terms “viral element” and “viral component” are used herein to refer to viral genes (e.g., genes encoding polymerase or structural proteins) or other elements of the viral genome (e.g., packaging signals, regulatory elements, LTRs, ITRs, etc.).

The term “oncolytic virus” is used herein to refer to a virus that is capable of infecting and replicating in a tumor cell such that the tumor cell may be killed. The oncolytic virus may be replication competent. As a non-limiting example, the oncolytic virus may comprise a rhabdovirus, i.e., any of a group of viruses comprising the family Rhabdoviridae, e.g., a vesicular stomatitis virus (VSV).

As used herein, the term “vesiculovirus” refers to any virus in the Vesiculovirus genus. Non-limiting examples of vesiculoviruses include Vesicular Stomatitis Virus (VSV) (e.g., VSV-New Jersey, VSV-Indiana), Alagoas vesiculovirus, Cocal vesiculovirus, Jurona vesiculovirus, Carajas vesiculovirus, Maraba vesiculovirus, Piry vesiculovirus, Calchaqui vesiculovirus, Yug Bogdanovac vesiculovirus, Isfahan vesiculovirus, Chandipura vesiculovirus, Perinct vesiculovirus, and Porton-S vesiculovirus. Vesicular Stomatitis Virus (VSV), in the Vesiculovirus genus, is a prototypic rhabdovirus. While VSV is used as an example in the present disclosure, this disclosure can also be used for other vesiculoviruses and other rhabdoviruses. There are two major serotypes of VSV, New Jersey and Indiana, both of which can infect insects and mammals, causing disease in cattle, equines, and swine. The VSV genome is composed of single-stranded, negative-sense RNA of 11-12 kb, which encodes five viral proteins: the nucleoprotein (N), the phosphoprotein (P), the matrix protein (M), the glycoprotein (G) and the viral polymerase (also known as large protein) (L). G monomers associate to form trimeric spikes anchored in the viral membrane.

The terms “vector”, “expression vector”, and “cloning vector” refer to any vehicle by which a nucleotide sequence, e.g., an RNA sequence or a DNA sequence, encoding for example, a foreign gene, may be introduced into a cell (e.g., a host cell) to genetically modify the cell and promote expression (e.g., transcription and translation) of said introduced nucleotide sequence. Non-limiting examples of vectors include synthesized RNA and DNA molecules plasmids, viruses, phages, and the like. In some embodiments, the vector may be a viral vector including, without limitation, a baculoviral vector, a herpes virus vector, a lentiviral vector, a retroviral vector, a vaccinia virus vector, an adeno-associated virus vector, an adenoviral vector, and an alphaviral vector.

The term “replication-competent” is used herein to refer to viruses (including wild-type and recombinant viruses) that are capable of infecting and propagating within a cell.

The term “pseudotyped” in connection with viral particles described herein refers to viral particles comprising in their lipid envelope or capsid molecules, e.g., proteins, glycoproteins, etc., which are mutated and/or heterologous compared to molecules typically found on the surface of the virus from which the particles are derived, and which may affect, contribute to, direct, redirect and/or completely change the tropism of the viral particle in comparison to a reference wild-type virus from which the viral particle is derived. In some embodiments, a viral particle is pseudotyped such that it recognizes, binds and/or infects a target (ligand or cell) that is different to that of a reference wild-type virus from which the viral particle is derived. In some embodiments, a viral particle is pseudotyped such that it does not recognize, bind, and/or infect a target (ligand or cell) of the reference wild-type virus from which the viral particle is derived.

The term “encoding” can refer to encoding from either the (+) or (−) sense strand of a polynucleotide, for example, for expression in the virus particle.

The terms “antibody” and “antibodies” refer to monoclonal antibodies, human antibodies, humanized antibodies, chimeric antibodies, single-chain Fvs (scFv), single chain antibodies, Fab fragments, F(ab′) fragments, disulfide-linked Fvs (sdFv), intrabodies, minibodies, diabodies and anti-idiotypic (anti-Id) antibodies (including, e.g., anti-Id antibodies to antigen specific TCR), and epitope-binding fragments of any of the above. The terms “antibody” and “antibodies” also refer to covalent diabodies such as those disclosed in U.S. Pat. Appl. Pub. 2007/0004909 and Ig-DARTS such as those disclosed in U.S. Pat. Appl. Pub. 2009/0060910. Antibodies useful in the present disclosure include immunoglobulin molecules and immunologically active fragments of immunoglobulin molecules, i.e., molecules that contain an antigen binding site. Immunoglobulin molecules can be of any type (e.g., IgG, IgE, IgM, IgD, IgA and IgY), class (e.g., IgG1, IgG2, IgG3, IgG4, IgA1 and IgA2) or subclass.

The term “T cell” or “T lymphocyte” is used herein in its broadest sense to refer to all types of immune cells expressing CD3, including, but not limited to, T-helper cells (CD4+ cells), cytotoxic T-cells (CD8+ cells), tumor infiltrating cytotoxic T cells (TIL; CD8+ T cell), CD4+CD8+ T cells, T-regulatory cells (Treg), and NK-T cells. T cells can include thymocytes, naïve T cells, memory T cells, immature T cells, mature T cells, resting T cells, or activated T cells. T cells may also include “gamma-delta T cells (γδ T cells),” which refer to a specialized population that to a small subset of T cells possessing a distinct TCR on their surface, and unlike the majority of T cells in which the TCR is composed of two glycoprotein chains designated α- and β-TCR chains, the TCR in γδ T cells is made up of ay-chain and a δ-chain.

The terms “Major Histocompatibility Complex”, “MHC” and “MHC molecule” encompass naturally occurring MHC molecules as well as individual chains of MHC molecules (e.g., MHC class I α (heavy) chain, β2-microglobulin, MHC class II α chain, MHC class II β chain), individual subunits of such chains of MHC molecules (e.g., α1, α2, and/or α3 subunits of MHC class I α chain, α1 and/or α2 subunits of MHC class II α chain, β1 and/or β2 subunits of MHC class II β chain) as well as fragments, mutants and various derivatives thereof (including fusion proteins), wherein such fragments, mutants and derivatives retain the ability to display an antigenic peptide for recognition by a TCR, e.g., an antigen-specific TCR. An MHC class I molecule comprises a peptide binding groove formed by the α1 and α2 domains of the heavy α chain that can stow a peptide of around 8-10 amino acids. Despite the fact that both classes of MHC bind a core of about 9 amino acids within peptides, the open-ended nature of MHC class II peptide-binding groove (the α1 domain of a class II MHC α polypeptide in association with the β1 domain of a class II MHC β polypeptide) allows for a wider range of peptide lengths. Peptides binding MHC class II usually vary between 13 and 17 amino acids in length, though shorter or longer lengths are not uncommon. As a result, peptides may shift within the MHC class II peptide-binding groove, changing which 9-mer sits directly within the groove at any given time. Conventional identifications of particular MHC variants are used herein. For example, HLA-B 17 refers to a human leucocyte antigen from the B gene group (hence a class I type MHC) gene position (known as a gene locus) number 17; gene HLA-DR11, refers to a human leucocyte antigen coded by a gene from the DR region (hence a class II type MHC) locus number 11.

The term “operably linked” or the like refers to a juxtaposition wherein the components described are in a relationship permitting them to function in their intended manner. For example, a control sequence “operably linked” to a coding sequence is ligated in such a way that expression of the coding sequence is achieved under conditions compatible with the control sequences. “Operably linked” sequences include both expression control sequences that are contiguous with a gene of interest and expression control sequences that act in trans or at a distance to control a gene of interest (or sequence of interest). The term “expression control sequence” includes polynucleotide sequences, which are necessary to affect the expression and processing of coding sequences to which they are ligated. “Expression control sequences” include: appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance polypeptide stability; and when desired, sequences that enhance polypeptide secretion. The nature of such control sequences differs depending upon the host organism. For example, in prokaryotes, such control sequences generally include promoter, ribosomal binding site and transcription termination sequence, while in eukaryotes typically such control sequences include promoters and transcription termination sequence. The term “control sequences” is intended to include components whose presence is essential for expression and processing and can also include additional components whose presence is advantageous, for example, leader sequences and fusion partner sequences.

The term “host cell” refers to any cell that comprises a heterologous nucleic acid. By way of a non-limiting example, the heterologous nucleic acid may be a vector. A host cell, for example, without limitation, may be a cell from any organism that is used, manipulated, modified, selected, transformed, or grown, for the production of a substance by the cell, e.g., the expression by the cell of, an RNA or DNA sequence, a gene, a protein, or an enzyme.

An “individual” or “subject” or “animal” refers to humans, veterinary animals (e.g., cats, dogs, cows, horses, sheep, pigs, etc.) and experimental animal models of diseases (e.g., mice, rats). In a preferred embodiment, the subject is a human.

The terms “nucleic acid,” “polynucleotide,” and “nucleotide” used interchangeably herein, include polymeric forms of nucleotides of any length, including ribonucleotides (RNA), deoxyribonucleotides (DNA), or analogs or modified versions thereof. They include single-, double-, and multi-stranded DNA or RNA, genomic DNA, cDNA, DNA-RNA hybrids, and polymers comprising purine bases, pyrimidine bases, or other natural, chemically modified, biochemically modified, non-natural, or derivatized nucleotide bases. A single-stranded nucleic acid can be the sense strand or the antisense strand.

Nucleic acids are said to have a “5′ end” and a “3′ end” because mononucleotides are reacted to make oligonucleotides in a manner such that the 5′ phosphate of one mononucleotide pentose ring is attached to the 3′ oxygen of its neighbor in one direction via a phosphodiester linkage. An end of an oligonucleotide is referred to as the “5′ end” if its 5′ phosphate is not linked to the 3′ oxygen of a mononucleotide pentose ring. An end of an oligonucleotide is referred to as the “3′ end” if its 3′ oxygen is not linked to a 5′ phosphate of another mononucleotide pentose ring. A nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements.

The term “fragment” when referring to a protein means a protein that is shorter or has fewer amino acids than the full-length protein. A fragment can be, for example, an N-terminal fragment (i.e., removal of a portion of the C-terminal end of the protein), a C-terminal fragment (i.e., removal of a portion of the N-terminal end of the protein), or an internal fragment. The term “fragment” when referring to a nucleic acid means a nucleic acid that is shorter or has fewer nucleotides than the full-length nucleic acid. A fragment can be, for example, a 5′ fragment (i.e., removal of a portion of the 3′ end of the nucleic acid), a 3′ fragment (i.e., removal of a portion of the 5′ end of the protein), or an internal fragment.

The term “derivative” as used herein refers to a nucleic acid, or protein, or a variant, or an analog thereof comprising one or more mutations and/or chemical modifications as compared to a corresponding full-length wild-type nucleic acid, or protein. Non-limiting examples of chemical modifications involving nucleic acids include, for example, modifications to the base moiety, sugar moiety, phosphate moiety, phosphate-sugar backbone, or a combination thereof.

“Sequence identity” or “identity” in the context of two polynucleotides or polypeptide sequences makes reference to the residues in the two sequences that are the same when aligned for maximum correspondence over a specified comparison window. When percentage of sequence identity is used in reference to proteins, residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known. Typically, this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE.

“Percentage of sequence identity” includes the value determined by comparing two optimally aligned sequences (greatest number of perfectly matched residues) over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity. Unless otherwise specified (e.g., the shorter sequence includes a linked heterologous sequence), the comparison window is the full length of the shorter of the two sequences being compared.

The terms “treat” or “treatment” of a state, disorder, disease, or condition include: (1) preventing, delaying, or reducing the incidence and/or likelihood of the appearance of at least one clinical or sub-clinical symptom of the state, disorder, disease, or condition developing in a subject that may be afflicted with or predisposed to the state, disorder, disease, or condition, but does not yet experience or display clinical or subclinical symptoms of the state, disorder, disease, or condition; or (2) inhibiting the state, disorder, disease, or condition, i.e., arresting, reducing, or delaying the development of the disease, or a relapse thereof, or at least one clinical or sub-clinical symptom thereof; or (3) relieving the state, disorder, disease, or condition, i.e., causing regression of the state, disorder, disease or condition or at least one of the clinical or sub-clinical symptoms of the state, disorder, disease or condition. The benefit to a subject to be treated is either statistically significant or at least perceptible to the patient or to the physician.

The term “effective” as applied to a dose or an amount refers to the quantity of a compound or pharmaceutical composition that is sufficient to result in a desired activity upon administration to a subject in need thereof. Note that when a combination of active ingredients is administered, the effective amount of the combination may or may not include amounts of each ingredient that would have been effective if administered individually. The exact amount required will vary from subject to subject, depending on the species, age, and general condition of the subject, the severity of the condition being treated, the particular drug or drugs employed, the mode of administration, and the like.

The phrase “pharmaceutically acceptable”, as used in connection with compositions described herein, refers to molecular entities and other ingredients of such compositions that are physiologically tolerable and do not typically produce untoward reactions when administered to a mammal (e.g., a human). Preferably, the term “pharmaceutically acceptable” means approved by a regulatory agency of the Federal or a state government or listed in the U.S. Pharmacopeia or other generally recognized pharmacopeia for use in mammals, and more particularly in humans.

The term “administration” and the like refers to and includes the administration of a composition to a subject or system (e.g., to a cell, organ, tissue, organism, or relevant component or set of components thereof). The skilled artisan will appreciate that route of administration may vary depending, for example, on the subject or system to which the composition is being administered, the nature of the composition, the purpose of the administration, etc. For example, in certain embodiments, administration to an animal subject (e.g., to a human or a rodent) may be bronchial (including by bronchial instillation), buccal, enteral, interdermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, vaginal and/or vitreal. In some embodiments, administration may involve intermittent dosing. In some embodiments, administration may involve continuous dosing (e.g., perfusion) for at least a selected period of time.

In accordance with the disclosure herein, there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition. Cold Spring Harbor, NY: Cold Spring Harbor Laboratory Press, 1989 (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984); Ausubel, F. M. et al. (eds.). Current Protocols in Molecular Biology. John Wiley & Sons, Inc., 1994. These techniques include site directed mutagenesis as described in Kunkel, Proc. Natl. Acad. Sci. USA 82: 488-492 (1985), U.S. Pat. No. 5,071,743, Fukuoka et al., Biochem. Biophys. Res. Commun. 263: 357-360 (1999); Kim and Maas, BioTech. 28: 196-198 (2000); Parikh and Guengerich, BioTech. 24: 4 28-431 (1998); Ray and Nickoloff, BioTech. 13: 342-346 (1992); Wang et al., BioTech. 19: 556-559 (1995); Wang and Malcolm, BioTech. 26: 680-682 (1999); Xu and Gong, BioTech. 26: 639-641 (1999), U.S. Pat. Nos. 5,789,166 and 5,932,419, Hogrefe, Strategies 14. 3: 74-75 (2001), U.S. Pat. Nos. 5,702,931, 5,780,270, and 6,242,222, Angag and Schutz, Biotech. 30: 486-488 (2001), Wang and Wilkinson, Biotech. 29: 976-978 (2000), Kang et al., Biotech. 20: 44-46 (1996), Ogel and McPherson, Protein Engineer. 5: 467-468 (1992), Kirsch and Joly, Nucl. Acids. Res. 26: 1848-1850 (1998), Rhem and Hancock, J. Bacteriol. 178: 3346-3349 (1996), Boles and Miogsa, Curr. Genet. 28: 197-198 (1995), Barrenttino et al., Nuc. Acids. Res. 22: 541-542 (1993), Tessier and Thomas, Meths. Molec. Biol. 57: 229-237, and Pons et al., Meth. Molec. Biol. 67: 209-218.

### Fusogenic Proteins

In one aspect, the present disclosure provides a recombinant fusogenic protein. Generally, a fusogenic protein may comprise a membrane-embedded polypeptide capable of mediating the fusion of two lipid membranes, at least one of which can incorporate the polypeptide. In some embodiments, the fusogenic protein described herein can comprise: (i) a rhabdovirus glycoprotein (G), or a functional fragment or derivative thereof, and (ii) a targeting molecule. The targeting molecule may be attached to the N-terminus of the rhabdovirus glycoprotein, or the functional fragment or derivative thereof, via a linker.

A rhabdovirus is member of the Rhabdoviridae family of viruses in the order Mononegavirales, encompassing more than 150 viruses of vertebrates, invertebrates, and plants. Examples of rhabdoviruses include rabies virus (RABV) from the Lyssavirus genus, vesiculoviruses from Vesiculovirus genus, the viral hemorrhagic septicemia virus (VHSV), and infectious hematopoietic necrosis virus, both from the Novirhabdovirus genus. Members of the genus Lyssavirus may cause lethal meningoencephalitis in humans and animals while VSV (genus Vesiculovirus) may cause symptoms clinically identical to those of foot-and-mouth disease in cattle and occasional, limited infections in humans. Dimarhabdoviruses are the supergroup of rhabdoviruses that infect mammals and mosquitoes.

Rhabdoviruses are bullet-shaped enveloped viruses with negative-sense single-stranded RNA genome 11-15 kb in length. The genome of rhabdoviruses can contain up to ten genes among which only five are common to all members of the family. These five common rhabdoviruses genes encode the nucleoprotein (N), the phosphoprotein (P), the matrix protein (M), the glycoprotein (G), and the viral polymerase (also known as large protein) (L). The rhabdoviruses genome associates with N, L, and P to form the nucleocapsid, which is condensed by the M protein into a tightly coiled helical structure. The condensed nucleocapsid is surrounded by a lipid bilayer containing the viral glycoprotein G that constitutes the spikes that protrude from the viral surface. Rhabdoviruses enter a host cell via the endocytic pathway and subsequently fuse with the cellular membrane within the acidic environment of the endosome. Both receptor recognition and membrane fusion are mediated by a single transmembrane viral glycoprotein (G). Fusion between the viral envelope and the endosomal membrane is triggered via a low-pH induced (in the endosome) structural rearrangement of the G resulting in the release the viral genome and associated proteins into the cytoplasm of target cells.

In some embodiments, the rhabdovirus described herein can be a vesiculovirus, or a functional fragment or derivative thereof. Examples of vesicoluviruses that can be used the present disclosure are listed in Table 1.

TABLE 1
Examples of Vesiculoviruses

| | Example source of virus in |
|---|---|
| Virus | nature |
| VSV-New Jersey | Mammals, mosquitoes, midges, |
| | blackflies, houseflies |
| VSV-Indiana | Mammals, mosquitoes, sandflies |
| Alagoas | Mammals, sandflies |
| Cocal | Mammals, mosquitoes, mites |
| Jurona | Mosquitoes |
| Carajas | Sandflies |
| Maraba | Sandflies |
| Piry | Mammals |
| Calchaqui | Mosquitoes |
| Yug Bogdanovac | Sandflies |
| Isfahan | Sandflies, ticks |
| Chandipura | Mammals, sandflies |
| Perinct | Mosquitoes, sandflies |
| Porton-S | Mosquitoes |

In some embodiments, the rhabdovirus disclosed herein may comprise a VSV. In some embodiments, the VSV can be a replication competent VSV. In some embodiments, VSV can be replication-incompetent.

A “fusogen” (e.g., a fusogenic protein such as a recombinant fusogenic protein described herein) or “fusogenic molecule” may refer to any molecule that can trigger membrane fusion when present on the surface of a virus. In some embodiments, a fusogen may act on the cell membrane to prevent spontaneous membrane fusion and promote fusion that may occur in a controlled and/or regulated manner. Upon activation, a fusogen may extend trimers anchored at one end by their transmembrane domains and expose an amphiphilic loop or hydrophobic fusion peptide that inserts into the target membrane. At this time, the two interacting domains are positioned in different membranes. Regulated refolding of the fusogenic complex into a hairpin-like structure then brings the fusion peptide and transmembrane domains to the same end of the molecule, which generates a pulling force that brings the two membranes into close (approximately 1 nm) apposition. The accumulated energy from this event can drive fusion through the formation of a hemifusion stalk-like connection, where only the contacting proximal leaflets of the membranes are fused while the inner leaflets remain intact. Expansion of the hemifusion stalk, and the subsequent fusion of the distal leaflets, completes the reaction by opening a fusion pore that permits the contents of the two compartments to mix. Fusion pore expansion is considered a final energy barrier before membrane fusion becomes permanent. Without wishing to be bound by theory, there is evidence that viral fusogens mediate fusion via hemifusion. Many different protein and non-protein fusogenic molecules may be used herein. In some embodiments, the fusogenic molecule is a fusogenic protein or polypeptide.

In some embodiments, a fusogen described herein may be capable of driving the fusion of a viral envelope of a virus described herein, e.g., a rhabdovirus such as but not limited to a VSV, with a cellular membrane of a target cell. In some embodiments, the fusogen may mediate the fusion of the plasma membrane of the target cell and/or one or more of a cell adjacent to the target cell (i.e., a neighboring cell(s)), thereby leading to the formation of a multinucleated cell, i.e., a syncytium. In some embodiments, a virus disclosed herein may trigger a cell-cell fusion, such as by way of a fusogen, which can form a syncytium. In some embodiments, the formation of a syncytium may occur during in vitro and/or in vivo infection by the virus. In some embodiments, the cell-cell fusion may allow for, for example, more efficient spread of the virus to a neighboring cell(s) than in the absence of such a cell-cell fusion. The spread of the virus to neighboring cells associated with the cell-cell fusion may allow for more efficient spread of the virus due to the absence of exposure of the virus to, e.g., neutralizing antibodies and/or other host immune responses and/or molecules. In various embodiments, the formation of a syncytium may permit viruses to evade or partially evade host defense mechanisms such as but not limited to humoral immune responses. In various embodiments, the formation of a syncytium may permit viruses to evade or partially evade restriction factors that target assembly and/or release of viral particles, and/or the entry of the virus into a target cell.

In some embodiments, the fusogenic molecule is a viral fusogenic molecule. Non-limiting examples of viral fusogenic molecules include, e.g., vesiculovirus fusogens (e.g., vesicular stomatitis virus G glycoprotein, alphavirus fusogens (e.g., a Sindbis virus glycoprotein), orthomyxovirus fusogens (e.g., influenza HA protein), paramyxovirus fusogens (e.g., a Nipah virus F protein or a measles virus F protein), as well as fusogens from Dengue virus (DV), Lassa fever virus, tick-borne encephalitis virus, Dengue virus, Hepatitis B virus, Rabies virus, Semliki Forest virus, Ross River virus, Aura virus, Borna disease virus, Hantaan virus, SARS-CoV virus, and various fragments, mutants, and derivatives thereof.

In some embodiments, the fusogenic molecule is heterologous to the virus from which the virus is derived. In some embodiments, the fusogenic molecule is a mutated protein which does not bind the fusogenic molecule's natural ligand(s).

There are two classes of viral fusogenic molecules and either may be used as targeting molecules. The class I fusogens trigger membrane fusion using helical coiled-coil structures, whereas the class II fusogens trigger fusion with 13 barrels. In some embodiments, class I fusogens are used. In other embodiments, class II fusogens are used. In still other embodiments, both class I and class II fusogens are used.

In some embodiments a recombinant fusogenic protein described herein can comprise a rhabdovirus glycoprotein (G) or functional fragment or derivative thereof. In some embodiments, the fusogenic molecule is a vesicular stomatitis virus (VSV) envelope protein. In certain embodiments, the recombinant fusogenic protein comprises the G protein of VSV (VSV-G or a fragment, mutant, derivative, or homolog thereof.). VSV-G can interact with a phospholipid component of the cell membrane to mediate viral entry by membrane fusion.

In some embodiments, the rhabdovirus G-protein described herein may include, without limitation, a vesicular stomatitis virus glycoprotein (VSV-G), a Flanders virus glycoprotein (FLAV-G), a Chandipura virus glycoprotein (CHPV-G), a Perinet virus glycoprotein (PERV-G), a Piry virus glycoprotein (PIRYV-G), a Fukuoka virus glycoprotein (FUKV-G), a Joinjakaka virus glycoprotein (JOIV-G), a Kumasi virus glycoprotein (KRV-G), a Isfahan glycoprotein (ISFV-G), a Jurona glycoprotein (JURV-G), a Mediterranean Bat glycoprotein (MBV-G), a Malpais Spring glycoprotein (MSPV-G), a Radi glycoprotein (RADV-G), a Rhinolophus affinis-G, a Yug Bugdanavoc glycoprotein (YBV-G), a Yinshui Bat glycoprotein (YSBV-G), a Keuraliba glycoprotein (KEUV-G), a Kimberley glycoprotein (KIMV-G), a Kanyawara glycoprotein (KYAV-G), a La Joya glycoprotein (LJV-G), a Mosquiero glycoprotein (MQOV-G), a Parry Creek glycoprotein (PCV-G), a Bas Congo glycoprotein (BASV-G), a Bovine Ephemeral fever glycoprotein (BEFV-G), a Curionopolis glycoprotein (CURV-G), a Drosophila melanogaster sigmavirus glycoprotein (DMelSV-G), a Niakha glycoprotein (NIAV-G), a Puerto almandras glycoprotein (PTAMV-G), or a Tupaia rhabdovirus (TUPTV-G), or a functional fragment or derivative thereof. Non-limiting examples of amino acid sequences of rhabdovirus G described herein are set forth in SEQ ID NO: 8-15, 17, 60-108, 157.

In some embodiments, the fusogenic protein can comprise a targeting molecule described herein. In some embodiments, the targeting molecule is attached to the N-terminus of the rhabdovirus glycoprotein. In some embodiments, the targeting molecule attached to the N-terminus may be attached to the rhabdovirus glycoprotein via a linker described herein.

In some embodiments, the N-terminus of the rhabdovirus G, or the functional fragment or derivative thereof, to which the targeting molecule can be attached, e.g., via a linker, does not comprise a rhabdovirus glycoprotein signal sequence. In some embodiments, the rhabdovirus G, or the functional fragment or derivative thereof, to which the targeting molecule can be attached is a mature rhabdovirus G which does not comprise, e.g., a rhabdovirus glycoprotein signal sequence. In some embodiments, the rhabdovirus glycoprotein signal sequence can be necessary to ensure that the rhabdovirus G comprising the signal sequence, e.g., a nascent rhabdovirus G, can enter the endoplasmic reticulum (ER). In some embodiments, the rhabdovirus glycoprotein signal sequence is cleaved from the nascent rhabdovirus G. Non-limiting examples of amino acid sequences of rhabdovirus G signal sequences are set forth in SEQ ID NO: 109-137. In some embodiments a rhabdovirus G comprises a signal sequence. Non-limiting examples of amino acid sequences of rhabdovirus glycoproteins comprising a signal sequence are set forth in SEQ ID NO: 80-108.

In some embodiments, the N-terminus of the rhabdovirus glycoprotein, or the functional fragment or derivative thereof, to which the targeting molecule can be attached, e.g., via a linker, does not comprise one or more amino acids present at the N-terminus of a mature wild-type (WT) rhabdovirus glycoprotein described herein. For instance, in certain embodiments, the N-terminus of the mature WT rhabdovirus glycoprotein, or the functional fragment or derivative thereof, to which the targeting molecule can be attached does not comprise 1 to 2, 1 to 4, 1 to 6, 1 to 8, 1 to 10, 1 to 12, 1 to 14, 1 to 16, 1 to 18, 1 to 20, 1 to 22, 1 to 24, 1 to 26, 1 to 28, 1 to 30, 1 to 32, 1 to 34, 1 to 36, 1 to 38, 1 to 40, 1 to 42, 1 to 44, 1 to 46, 1 to 48, or 1 to 50, or more, amino acids present at the N-terminus of a mature WT rhabdovirus glycoprotein described herein. In some embodiments, the N-terminus of the mature WT rhabdovirus glycoprotein, or the functional fragment or derivative thereof, to which the targeting molecule can be attached does not comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, or more, amino acids present at the N-terminus of a mature WT rhabdovirus glycoprotein described herein. In some embodiments, the mature WT rhabdovirus glycoprotein, or functional fragment or derivative thereof can be a mature WT vesicular stomatitis virus glycoprotein (VSV-G), or a functional fragment or derivative thereof, described herein.

In some embodiments, the N-terminus of the rhabdovirus glycoprotein, or the functional fragment or derivative thereof, to which the targeting molecule can be attached, e.g., via a linker, does not comprise one or more amino acids which can be encoded by the 5′ end of a wild-type (WT) glycoprotein gene, e.g., a signal sequence (e.g., a rhabdovirus glycoprotein signal sequence), or fragment or derivative thereof described herein, which can be encoded by the 5′ end of a wild-type (WT) glycoprotein gene. In some embodiments, when the N-terminus of the rhabdovirus glycoprotein, or functional fragment or derivative thereof, does not comprise one or more amino acids encoded by the 5′ end of the WT glycoprotein gene (e.g., a signal sequence), the rhabdovirus glycoprotein can comprise a mature rhabdovirus glycoprotein, i.e., a rhabdovirus glycoprotein which does not comprise a signal peptide described herein. In certain embodiments, the N-terminus of the mature WT rhabdovirus glycoprotein, or the functional fragment or derivative thereof, to which the targeting molecule can be attached does not comprise 1 to 2, 1 to 4, 1 to 6, 1 to 8, 1 to 10, 1 to 12, 1 to 14, 1 to 16, 1 to 18, 1 to 20, 1 to 22, 1 to 24, 1 to 26, 1 to 28, 1 to 30, 1 to 32, 1 to 34, 1 to 36, 1 to 38, 1 to 40, 1 to 42, 1 to 44, 1 to 46, 1 to 48, or 1 to 50, or more, amino acids encoded by the 5′ end of the WT glycoprotein gene. In some embodiments, the N-terminus of the mature WT rhabdovirus glycoprotein, or the functional fragment or derivative thereof, to which the targeting molecule can be attached does not comprise approximately 10-32 amino acids encoded by the 5′ end of the WT glycoprotein gene. In some embodiments, the N-terminus of the mature WT rhabdovirus glycoprotein, or the functional fragment or derivative thereof, to which the targeting molecule can be attached does not comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, or more, amino acids encoded by the 5′ end of the WT glycoprotein gene. In some embodiments, when the N-terminus of the rhabdovirus glycoprotein, or functional fragment or derivative thereof, does not comprise one or more amino acids encoded by the 5′ end of the WT glycoprotein gene, the rhabdovirus glycoprotein can comprise a mature rhabdovirus glycoprotein which does not comprise a signal peptide sequence such as, for example, a signal sequence set forth in SEQ ID NO: 109-137, described herein. Non-limiting examples of amino acid sequences of mature rhabdovirus glycoproteins which do not comprise a signal sequence are set forth in SEQ ID NO: 8-15, 17, 60-79. In some embodiments, the mature WT rhabdovirus glycoprotein, or functional fragment or derivative thereof can be a mature WT vesicular stomatitis virus glycoprotein (VSV-G), or a functional fragment or derivative thereof, described herein.

In some embodiments, a fusogenic protein described herein may comprise a VSV-G. In some embodiments, the VSV-G comprises the amino acid sequence of SEQ ID NO: 8 or 80, or a functional fragment or derivative thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity with SEQ ID NO: 8 or 80. In certain embodiments, the nucleotide sequence that encodes the VSV-G comprises the nucleotide sequence that encodes the amino acid sequence of SEQ ID NO: 8 or 80, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 8 or 80. In certain embodiments, the VSV-G comprises the amino acid sequence of SEQ ID NO: 8 or 80.

In some embodiments, a recombinant fusogenic protein described herein comprises a rhabdovirus glycoprotein from VSV-G. In some embodiments, the VSV-G comprises the sequence SEQ ID NO: 8. In some embodiments, the VSV-G consists of the sequence SEQ ID NO: 8.

In some embodiments, a VSV-G described herein may be derived, for example, from a VSV-G as described in U.S. Patent Publication No. 2020/0216502, the content of which is incorporated herein by reference in its entirety for all purposes. As an example, without limitation, the VSV-G, or functional fragment or derivative thereof may comprise the amino acid sequence of SEQ ID NO: 157 or a functional fragment or derivative thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity with SEQ ID NO: 157.

In some embodiments, the fusogenic protein, or the functional fragment or derivative thereof described herein, can comprise a rhabdovirus glycoprotein (G) comprising one or more mutations compared to the corresponding WT sequence of the rhabdovirus glycoprotein.

In some embodiments, the rhabdovirus glycoprotein, or the functional fragment or derivative thereof, described herein may comprise amino acid mutation(s) in one or more positions. Non-limiting examples of amino acid mutations comprise amino acid substitutions, insertions, and/or deletions. Amino acid substitution can mean that an amino acid residue is substituted for a replacement amino acid residue at the same position. Inserted amino acid residues may be inserted at any position and may be inserted such that some or all of the inserted amino acid residues are immediately adjacent one another or may be inserted such that none of the inserted amino acid residues is immediately adjacent to another inserted amino acid residue.

In some embodiments, the rhabdovirus glycoprotein, or the functional fragment or derivative thereof, may comprise amino acid mutation(s) in one or more positions. As a non-limiting example, a rhabdovirus glycoprotein, or the functional fragment or derivative thereof, may comprise an amino acid mutation(s) in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, or 700, or more amino acid positions.

In some embodiments, the rhabdovirus protein, or fragment or derivative thereof, may comprise one or more mutations, i.e., amino acid substitutions, insertions, or deletions, or combination thereof, at one or more location in its amino acid sequence as compared to an amino acid sequence of a WT rhabdovirus protein. For example, a VSV-G protein described herein may include a substitution(s) of one or more amino acids in the amino acid sequence of a parent VSV-G protein with a similar or homologous amino acid(s) or a dissimilar amino acid(s).

In some embodiments, a rhabdovirus G protein described herein may include any amino acid sequence having an identity of at least about 50% or more, about 60% or more, about 70% or more, 71% or more, 72% or more, 73% or more, 74% or more, 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, 99% or more, 99.1% or more, 99.2% or more, 99.3% or more, 99.4% or more, 99.5% or more, 99.6% or more, 99.7% or more, 99.8% or more, 99.9% or more, or 100% to its WT sequence, and having the activity of the WT sequence.

In some embodiments, a VSV-G polypeptide, or a functional fragment or derivative thereof described herein, can comprise one or more mutations compared to its corresponding WT VSV-G sequence (see, e.g., SEQ ID NO: 8). In some embodiments, the one or more mutations in the VSV-G may, for example, reduce or eliminate binding of the VSV-G protein, or the functional fragment or derivative thereof, to low-density lipoprotein receptor (LDLR).

In some embodiments, the one or more mutations in a VSV-G polypeptide, or the functional fragment or derivative thereof, comprises one or more amino acid substitutions and/or deletions at positions corresponding to positions H8, K47, Y209, and/or R354 in SEQ ID NO: 8. In some embodiments, the one or more mutations in a VSV-G polypeptide, or the functional fragment or derivative thereof, may comprise, for example, one or more amino acid substitutions and/or deletions at amino acid positions corresponding to positions 8, or 47, or 209, or 354; or both positions 8 and 47; or both positions 8 and 209; or both positions 8 and 354; or both positions 47 and 209; or both positions 47 and 354; or both positions 209 and 354; or positions 8 and 47 and 209, combined; or positions 8 and 47 and 354, combined; or positions 8 and 209 and 354, combined; or positions 47 and 209 and 354, combined; or positions 8 and 47 and 209 and 354, combined, in SEQ ID NO: 8.

In various embodiments, a mutant VSV-G polypeptide may comprise, for example, a mutation where an amino acid at position 8 can be substituted with any amino acid apart from H, and preferably apart from Y; the amino acid position 47 can be substituted with any amino acid apart from K; the amino acid at position 209 can be substituted with any amino acid apart from Y and preferably apart from H; and/or the amino acid at position 354 can substituted by any amino acid apart from R, in SEQ ID NO: 8.

In some embodiments, a mutant VSV-G polypeptide described herein can comprise a fusogen that comprises the sequence SEQ ID NO: 8, with amino acid substitutions at positions (i) K47, (ii) R354, and (iii) H8 or Y209.

In some embodiments, a mutant VSV-G polypeptide described herein can consist of the sequence SEQ ID NO: 8, with amino acid substitutions at positions (i) K47, (ii) R354, and (iii) H8 or Y209.

In some embodiments, a mutant VSV-G polypeptide described herein can comprise a fusogen that comprises the sequence SEQ ID NO: 8, with amino acid substitutions at positions K47, R354, H8, and Y209.

In some embodiments, a mutant VSV-G polypeptide described herein can consist of the sequence SEQ ID NO: 8, with amino acid substitutions at positions K47, R354, H8, and Y209.

In some embodiments, the one or more mutations in a VSV-G polypeptide, or the functional fragment or derivative thereof, comprise an amino acid deletion at one or more positions corresponding to H8, K47, Y209, and/or R354 in SEQ ID NO: 8.

In some embodiments, a mutant VSV-G polypeptide described herein can comprise SEQ ID NO: 8, or a functional fragment of derivative thereof, with amino acid deletions at positions (i) K47, (ii) R354, and (iii) H8 or Y209.

In some embodiments, a mutant VSV-G polypeptide herein can consist of the sequence SEQ ID NO: 8, with amino acid deletions at positions (i) K47, (ii) PR54, and (iii) H8 or Y209.

In some embodiments, a mutant VSV-G polypeptide described herein can comprise the sequence SEQ ID NO: 8, with an amino acid deletion at position K47.

In some embodiments, a mutant VSV-G polypeptide described herein can consist of the sequence SEQ ID NO: 8, with an amino acid deletion at positions K47.

Other exemplary mutations in the VSV-G that can reduce or eliminate binding of the VSV-G protein, or the functional fragment or derivative thereof, to low-density lipoprotein receptor (LDLR) are described in US 2020/0216502, which is incorporated herein by reference in its entirety.

In some embodiments, a VSV-G, or the functional fragment or derivative thereof, may comprise one or more viral titer increasing mutations. Non-limiting examples of viral titer increasing mutations are M184T and F250L, as specified relative to positions within SEQ ID NO: 8. In some embodiments, a VSV-G, or functional fragment or derivative thereof, can comprises one or more viral titer increasing mutations in the VSV-G such as but not limited to M184T and/or F250L, as specified relative to positions within SEQ ID NO: 8.

Other examples of viral titer increasing mutations include those described in US 2022/0162266, which is incorporated herein by reference in its entirety, such as H22N and S422I in the ectodomain of VSV Indiana glycoprotein G (or similar substitutions, such as S422F, S422M, S422L or S422V, or equivalent substitutions in equivalent positions of G ectodomain of other VSV strains).

In various embodiments, a recombinant fusogenic protein of the present disclosure comprises a fusogen that has least at least 50%, at least 60%, at least 70%, at least 80%, at least 90% or 100% amino acid sequence identity to vesicular stomatitis virus glycoprotein (VSV-G) comprising the sequence of SEQ ID NO: 8, or a functional fragment or derivative thereof. In various embodiments, a recombinant fusogenic protein of the present disclosure comprises a fusogen comprising a vesicular stomatitis virus glycoprotein (VSV-G) consisting of the sequence SEQ ID NO: 8. In various embodiments, a recombinant fusogenic protein of the present disclosure comprises a fusogen that has least at least 60% amino acid sequence identity to vesicular stomatitis virus glycoprotein (VSV-G) comprising the sequence of SEQ ID NO: 8, or a functional fragment or derivative thereof. In such embodiments, the fusogen, or the functional fragment or derivative thereof, may comprise one or more amino acid deletions at one or more positions corresponding to positions H8, K47, Y209, and/or R354 in SEQ ID NO: 8. In some embodiments, the fusogen comprises SEQ ID NO: 8, or a functional fragment or derivative thereof, with one or more amino acid deletions at one or more positions selected from of H8, K47, Y209, and/or R354. In some embodiments the fusogen, or the functional fragment or derivative thereof, may further comprise one or more viral titer increasing mutations such as but not limited to viral titer increasing mutations at one or more positions corresponding to positions M184 and F250 of SEQ ID NO: 8. In some embodiments, the one or more viral titer increasing mutations in a VSV-G, or the functional fragment or derivative thereof, can be, e.g., M184T and/or F250L, as specified relative to positions in SEQ ID NO: 8.

In some embodiments, a recombinant fusogenic protein described herein comprises a fusogen comprising an N-terminus which may be attached to a targeting molecule, e.g., by way of a linker. In some embodiments, the recombinant fusogen protein further comprises a targeting molecule located at the N-terminus of the fusogen. In some embodiments, the targeting molecule is attached to the N-terminus of the fusogen, or functional or derivative thereof, via a linker.

In some embodiments, the N-terminus of the fusogen does not comprise a signal sequence described herein and/or does not comprise one or more amino acids present at the N-terminus of a mature WT fusogen. By way of a non-limiting example, the N-terminus of the mature WT fusogen, or the functional fragment or derivative thereof, to which the targeting molecule can be attached does not comprise 1 to 2, 1 to 4, 1 to 6, 1 to 8, 1 to 10, 1 to 12, 1 to 14, 1 to 16, 1 to 18, 1 to 20, 1 to 22, 1 to 24, 1 to 26, 1 to 28, 1 to 30, 1 to 32, 1 to 34, 1 to 36, 1 to 38, 1 to 40, 1 to 42, 1 to 44, 1 to 46, 1 to 48, or 1 to 50, or more, amino acids present at the N-terminus of a mature WT fusogen described herein. In some embodiments, the N-terminus of the mature WT fusogen, or the functional fragment or derivative thereof, to which the targeting molecule can be attached does not comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, or more, amino acids present at the N-terminus of a mature WT fusogen described herein.

In some embodiments, a recombinant fusogenic protein described herein may comprise a rhabdovirus glycoprotein derived from a Flanders virus (FLAV-G). In some embodiments, the FLAV-G comprises the amino acid sequence of SEQ ID NO: 9 or 81, or a functional fragment or derivative thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity with SEQ ID NO: 9 or 81. In certain embodiments, the nucleotide sequence that encodes the FLAV-G comprises the nucleotide sequence that encodes the amino acid sequence of SEQ ID NO: 9 or 81, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 9 or 81. In certain embodiments, the FLAV-G comprises the amino acid sequence of SEQ ID NO: 9 or 81.

In some embodiments, a recombinant fusogenic protein described herein comprises a rhabdovirus glycoprotein from FLAV-G. In some embodiments, the FLAV-G comprises the sequence SEQ ID NO: 9. In some embodiments, the FLAV-G consists of the sequence SEQ ID NO: 9.

In some embodiments, a recombinant fusogenic protein described herein may comprise a rhabdovirus glycoprotein derived from a Chandipura virus (CHPV-G). In some embodiments, the CHPV-G comprises the amino acid sequence of SEQ ID NO: 10 or 82, or a functional fragment or derivative thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity with SEQ ID NO: 10 or 82. In certain embodiments, the nucleotide sequence that encodes the CHPV-G comprises the nucleotide sequence that encodes the amino acid sequence of SEQ ID NO: 10 or 82, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 10 or 82. In certain embodiments, the CHPV-G comprises the amino acid sequence of SEQ ID NO: 10 or 82.

In some embodiments, a recombinant fusogenic protein described herein comprises a rhabdovirus glycoprotein from CHPV-G. In some embodiments, the CHPV-G comprises the sequence SEQ ID NO: 10. In some embodiments, the CHPV-G consists of the sequence SEQ ID NO: 10.

In some embodiments, a recombinant fusogenic protein described herein may comprise a rhabdovirus glycoprotein derived from a Perinet virus (PERV-G). In some embodiments, the PERV-G comprises the amino acid sequence of SEQ ID NO: 11 or 87, or a functional fragment or derivative thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity with SEQ ID NO: 11 or 87. In certain embodiments, the nucleotide sequence that encodes the PERV-G comprises the nucleotide sequence that encodes the amino acid sequence of SEQ ID NO: 11 or 87, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 11 or 87. In certain embodiments, the PERV-G comprises the amino acid sequence of SEQ ID NO: 11 or 87.

In some embodiments, a recombinant fusogenic protein described herein comprises a rhabdovirus glycoprotein from PERV-G. In some embodiments, the PERV-G comprises the sequence SEQ ID NO: 11 In some embodiments, the PERV-G consists of the sequence SEQ ID NO: 11.

In some embodiments, a recombinant fusogenic protein described herein may comprise a rhabdovirus glycoprotein derived from a Piry virus (PIRYV-G). In some embodiments, the PIRYV-G comprises the amino acid sequence of SEQ ID NO: 12 or 88, or a functional fragment or derivative thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity with SEQ ID NO: 12 or 88. In certain embodiments, the nucleotide sequence that encodes the PIRYV-G comprises the nucleotide sequence that encodes the amino acid sequence of SEQ ID NO: 12 or 88, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 12 or 88. In certain embodiments, the PIRYV-G comprises the amino acid sequence of SEQ ID NO: 12 or 88.

In some embodiments, a recombinant fusogenic protein described herein comprises a rhabdovirus glycoprotein from PIRYV-G. In some embodiments, the PIRYV-G comprises the sequence SEQ ID NO: 12. In some embodiments, the PIRYV-G consists of the sequence SEQ ID NO: 12.

In some embodiments, a recombinant fusogenic protein described herein may comprise a rhabdovirus glycoprotein derived from a Fukuoka virus (FUKV-G). In some embodiments, the FUKV-G comprises the amino acid sequence of SEQ ID NO: 13 or 93, or a functional fragment or derivative thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity with SEQ ID NO: 13 or 93. In certain embodiments, the nucleotide sequence that encodes the FUKV-G comprises the nucleotide sequence that encodes the amino acid sequence of SEQ ID NO: 13 or 93, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 13 or 93. In certain embodiments, the FUKV-G comprises the amino acid sequence of SEQ ID NO: 13 or 93.

In some embodiments, a recombinant fusogenic protein described herein comprises a rhabdovirus glycoprotein from FUKV-G. In some embodiments, the FUKV-G comprises the sequence SEQ ID NO: 13. In some embodiments, the FUKV-G consists of the sequence SEQ ID NO: 13.

In some embodiments, a recombinant fusogenic protein described herein may comprise a rhabdovirus glycoprotein derived from a Joinjakaka virus (JOIV-G). In some embodiments, the JOIV-G comprises the amino acid sequence of SEQ ID NO: 14 or 94, or a functional fragment or derivative thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity with SEQ ID NO: 14 or 94. In certain embodiments, the nucleotide sequence that encodes the JOIV-G comprises the nucleotide sequence that encodes the amino acid sequence of SEQ ID NO: 14 or 94, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 14 or 94. In certain embodiments, the JOIV-G comprises the amino acid sequence of SEQ ID NO: 14 or 94.

In some embodiments, a recombinant fusogenic protein described herein comprises a rhabdovirus glycoprotein from JOIV-G. In some embodiments, the JOIV-G comprises the sequence SEQ ID NO: 14. In some embodiments, the JOIV-G consists of the sequence SEQ ID NO: 14.

In some embodiments, a recombinant fusogenic protein described herein may comprise a rhabdovirus glycoprotein derived from a Kumasi virus (KRV-G). In some embodiments, the KRV-G comprises the amino acid sequence of SEQ ID NO: 15 or 97, or a functional fragment or derivative thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity with SEQ ID NO: 15 or 97. In certain embodiments, the nucleotide sequence that encodes the KRV-G comprises the nucleotide sequence that encodes the amino acid sequence of SEQ ID NO: 15 or 97, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 15 or 97. In certain embodiments, the KRV-G comprises the amino acid sequence of SEQ ID NO: 15 or 97.

In some embodiments, a recombinant fusogenic protein described herein comprises a rhabdovirus glycoprotein from KRV-G. In some embodiments, the KRV-G comprises the sequence SEQ ID NO: 15. In some embodiments, the KRV-G consists of the sequence SEQ ID NO: 15.

In some embodiments, a recombinant fusogenic protein described herein may comprise a rhabdovirus glycoprotein derived from a Keuraliba virus (KEUV-G). In some embodiments, the KEUV-G comprises the amino acid sequence of SEQ ID NO: 17 or 95, or a functional fragment or derivative thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity with SEQ ID NO: 17 or 95. In certain embodiments, the nucleotide sequence that encodes the KEUV-G comprises the nucleotide sequence that encodes the amino acid sequence of SEQ ID NO: 17 or 95, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 17 or 95. In certain embodiments, the KEUV-G comprises the amino acid sequence of SEQ ID NO: 17 or 95.

In some embodiments, a recombinant fusogenic protein described herein comprises a rhabdovirus glycoprotein from KEUV-G. In some embodiments, the KEUV-G comprises the sequence SEQ ID NO: 17. In some embodiments, the KEUV-G consists of the sequence SEQ ID NO: 17.

In some embodiments, a recombinant fusogenic protein described herein may comprise a rhabdovirus glycoprotein derived from an Isfahan virus (ISFV-G). In some embodiments, the ISFV-G comprises the amino acid sequence of SEQ ID NO: 60 or 83, or a functional fragment or derivative thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity with SEQ ID NO: 60 or 83. In certain embodiments, the nucleotide sequence that encodes the ISFV-G comprises the nucleotide sequence that encodes the amino acid sequence of SEQ ID NO: 60 or 83, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 60 or 83. In certain embodiments, the ISFV-G comprises the amino acid sequence of SEQ ID NO: 60 or 83.

In some embodiments, a recombinant fusogenic protein described herein comprises a rhabdovirus glycoprotein from ISFV-G. In some embodiments, the ISFV-G comprises the sequence SEQ ID NO: 60. In some embodiments, the ISFV-G consists of the sequence SEQ ID NO: 60.

In some embodiments, a recombinant fusogenic protein described herein may comprise a rhabdovirus glycoprotein derived from an Jurona virus (JURV-G). In some embodiments, the JURV-G comprises the amino acid sequence of SEQ ID NO: 61 or 84, or a functional fragment or derivative thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity with SEQ ID NO: 61 or 84. In certain embodiments, the nucleotide sequence that encodes the JURV-G comprises the nucleotide sequence that encodes the amino acid sequence of SEQ ID NO: 61 or 84, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 61 or 84. In certain embodiments, the JURV-G comprises the amino acid sequence of SEQ ID NO: 61 or 84.

In some embodiments, a recombinant fusogenic protein described herein comprises a rhabdovirus glycoprotein from JURV-G. In some embodiments, the JURV-G comprises the sequence SEQ ID NO: 61. In some embodiments, the JURV-G consists of the sequence SEQ ID NO: 61.

In some embodiments, a recombinant fusogenic protein described herein may comprise a rhabdovirus glycoprotein derived from a Mediterranean Bat virus (MBV-G). In some embodiments, the MBV-G comprises the amino acid sequence of SEQ ID NO: 62 or 85, or a functional fragment or derivative thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity with SEQ ID NO: 62 or 85. In certain embodiments, the nucleotide sequence that encodes the MBV-G comprises the nucleotide sequence that encodes the amino acid sequence of SEQ ID NO: 62 or 85, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 62 or 85. In certain embodiments, the MBV-G comprises the amino acid sequence of SEQ ID NO: 62 or 85.

In some embodiments, a recombinant fusogenic protein described herein comprises a rhabdovirus glycoprotein from MBV-G. In some embodiments, the MBV-G comprises the sequence SEQ ID NO: 62. In some embodiments, the MBV-G consists of the sequence SEQ ID NO: 62.

In some embodiments, a recombinant fusogenic protein described herein may comprise a rhabdovirus glycoprotein derived from a Malpais Spring virus (MSPV-G). In some embodiments, the MSPV-G comprises the amino acid sequence of SEQ ID NO: 63 or 86, or a functional fragment or derivative thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity with SEQ ID NO: 63 or 86. In certain embodiments, the nucleotide sequence that encodes the MSPV-G comprises the nucleotide sequence that encodes the amino acid sequence of SEQ ID NO: 63 or 86, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 63 or 86. In certain embodiments, the MSPV-G comprises the amino acid sequence of SEQ ID NO: 63 or 86.

In some embodiments, a recombinant fusogenic protein described herein comprises a rhabdovirus glycoprotein from MSPV-G. In some embodiments, the MSPV-G comprises the sequence SEQ ID NO: 63. In some embodiments, the MSPV-G consists of the sequence SEQ ID NO: 63.

In some embodiments, a recombinant fusogenic protein described herein may comprise a rhabdovirus glycoprotein derived from a Radi virus (RADV-G). In some embodiments, the RADV-G comprises the amino acid sequence of SEQ ID NO: 64 or 89, or a functional fragment or derivative thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity with SEQ ID NO: 64 or 89. In certain embodiments, the nucleotide sequence that encodes the RADV-G comprises the nucleotide sequence that encodes the amino acid sequence of SEQ ID NO: 64 or 89, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 64 or 89. In certain embodiments, the RADV-G comprises the amino acid sequence of SEQ ID NO: 64 or 89.

In some embodiments, a recombinant fusogenic protein described herein comprises a rhabdovirus glycoprotein from RADV-G. In some embodiments, the RADV-G comprises the sequence SEQ ID NO: 64. In some embodiments, the RADV-G consists of the sequence SEQ ID NO: 64.

In some embodiments, a recombinant fusogenic protein described herein may comprise a rhabdovirus glycoprotein derived from a Rhinolophus affinis virus (Rhinolophus affinis G). In some embodiments, the Rhinolophus affinis G comprises the amino acid sequence of SEQ ID NO: 65 or 90, or a functional fragment or derivative thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity with SEQ ID NO: 65 or 90. In certain embodiments, the nucleotide sequence that encodes the Rhinolophus affinis G comprises the nucleotide sequence that encodes the amino acid sequence of SEQ ID NO: 65 or 90, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 65 or 90. In certain embodiments, the Rhinolophus affinis G comprises the amino acid sequence of SEQ ID NO: 65 or 90.

In some embodiments, a recombinant fusogenic protein described herein comprises a rhabdovirus glycoprotein from Rhinolophus affinis G. In some embodiments, the Rhinolophus affinis G comprises the sequence SEQ ID NO: 65. In some embodiments, the Rhinolophus affinis G consists of the sequence SEQ ID NO: 65.

In some embodiments, a recombinant fusogenic protein described herein may comprise a rhabdovirus glycoprotein derived from a Yug Bugdanavoc virus (YBV-G). In some embodiments, the YBV-G comprises the amino acid sequence of SEQ ID NO: 66 or 91, or a functional fragment or derivative thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity with SEQ ID NO: 66 or 91. In certain embodiments, the nucleotide sequence that encodes the YBV-G comprises the nucleotide sequence that encodes the amino acid sequence of SEQ ID NO: 66 or 91, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 66 or 91. In certain embodiments, the YBV-G comprises the amino acid sequence of SEQ ID NO: 66 or 91.

In some embodiments, a recombinant fusogenic protein described herein comprises a rhabdovirus glycoprotein from YBV-G. In some embodiments, the YBV-G comprises the sequence SEQ ID NO: 66. In some embodiments, the YBV-G consists of the sequence SEQ ID NO: 66.

In some embodiments, a recombinant fusogenic protein described herein may comprise a rhabdovirus glycoprotein derived from a Yinshui Bat virus (YSBV-G). In some embodiments, the YSBV-G comprises the amino acid sequence of SEQ ID NO: 67 or 92, or a functional fragment or derivative thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity with SEQ ID NO: 67 or 92. In certain embodiments, the nucleotide sequence that encodes the YSBV-G comprises the nucleotide sequence that encodes the amino acid sequence of SEQ ID NO: 67 or 92, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 67 or 92. In certain embodiments, the YSBV-G comprises the amino acid sequence of SEQ ID NO: 67 or 92.

In some embodiments, a recombinant fusogenic protein described herein comprises a rhabdovirus glycoprotein from YSBV-G. In some embodiments, the YSBV-G comprises the sequence SEQ ID NO: 67. In some embodiments, the YSBV-G consists of the sequence SEQ ID NO: 67.

In some embodiments, a recombinant fusogenic protein described herein may comprise a rhabdovirus glycoprotein derived from a Kimberley virus (KIMV-G). In some embodiments, the KIMV-G comprises the amino acid sequence of SEQ ID NO: 68 or 96, or a functional fragment or derivative thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity with SEQ ID NO: 68 or 96. In certain embodiments, the nucleotide sequence that encodes the KIMV-G comprises the nucleotide sequence that encodes the amino acid sequence of SEQ ID NO: 68 or 96, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 68 or 96. In certain embodiments, the KIMV-G comprises the amino acid sequence of SEQ ID NO: 68 or 96.

In some embodiments, a recombinant fusogenic protein described herein comprises a rhabdovirus glycoprotein from KIMV-G. In some embodiments, the KIMV-G comprises the sequence SEQ ID NO: 68. In some embodiments, the KIMV-G consists of the sequence SEQ ID NO: 68.

In some embodiments, a recombinant fusogenic protein described herein may comprise a rhabdovirus glycoprotein derived from a Kanyawara virus (KYAV-G). In some embodiments, the KYAV-G comprises the amino acid sequence of SEQ ID NO: 69 or 98, or a functional fragment or derivative thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity with SEQ ID NO: 69 or 98. In certain embodiments, the nucleotide sequence that encodes the KYAV-G comprises the nucleotide sequence that encodes the amino acid sequence of SEQ ID NO: 69 or 98, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 69 or 98. In certain embodiments, the KYAV-G comprises the amino acid sequence of SEQ ID NO: 69 or 98.

In some embodiments, a recombinant fusogenic protein described herein comprises a rhabdovirus glycoprotein from KYAV-G. In some embodiments, the KYAV-G comprises the sequence SEQ ID NO: 69. In some embodiments, the KYAV-G consists of the sequence SEQ ID NO: 69.

In some embodiments, a recombinant fusogenic protein described herein may comprise a rhabdovirus glycoprotein derived from a La Joya virus (LJV-G). In some embodiments, the LJV-G comprises the amino acid sequence of SEQ ID NO: 70 or 99, or a functional fragment or derivative thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity with SEQ ID NO: 70 or 99. In certain embodiments, the nucleotide sequence that encodes the LJV-G comprises the nucleotide sequence that encodes the amino acid sequence of SEQ ID NO: 70 or 99, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 70 or 99. In certain embodiments, the LJV-G comprises the amino acid sequence of SEQ ID NO: 70 or 99.

In some embodiments, a recombinant fusogenic protein described herein comprises a rhabdovirus glycoprotein from LJV-G. In some embodiments, the LJV-G comprises the sequence SEQ ID NO: 70. In some embodiments, the LJV-G consists of the sequence SEQ ID NO: 70.

In some embodiments, a recombinant fusogenic protein described herein may comprise a rhabdovirus glycoprotein derived from a Mosquiero virus (MQOV-G). In some embodiments, the MQOV-G comprises the amino acid sequence of SEQ ID NO: 71 or 100, or a functional fragment or derivative thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity with SEQ ID NO: 71 or 100. In certain embodiments, the nucleotide sequence that encodes the MQOV-G comprises the nucleotide sequence that encodes the amino acid sequence of SEQ ID NO: 71 or 100, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 71 or 100. In certain embodiments, the MQOV-G comprises the amino acid sequence of SEQ ID NO: 71 or 100.

In some embodiments, a recombinant fusogenic protein described herein comprises a rhabdovirus glycoprotein from MQOV-G. In some embodiments, the MQOV-G comprises the sequence SEQ ID NO: 71. In some embodiments, the MQOV-G consists of the sequence SEQ ID NO: 71.

In some embodiments, a recombinant fusogenic protein described herein may comprise a rhabdovirus glycoprotein derived from a Parry Creek virus (PCV-G). In some embodiments, the PCV-G comprises the amino acid sequence of SEQ ID NO: 72 or 101, or a functional fragment or derivative thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity with SEQ ID NO: 72 or 101. In certain embodiments, the nucleotide sequence that encodes the PCV-G comprises the nucleotide sequence that encodes the amino acid sequence of SEQ ID NO: 72 or 101, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 72 or 101. In certain embodiments, the PCV-G comprises the amino acid sequence of SEQ ID NO: 72 or 101.

In some embodiments, a recombinant fusogenic protein described herein comprises a rhabdovirus glycoprotein from PCV-G. In some embodiments, the PCV-G comprises the sequence SEQ ID NO: 72. In some embodiments, the PCV-G consists of the sequence SEQ ID NO: 72.

In some embodiments, a recombinant fusogenic protein described herein may comprise a rhabdovirus glycoprotein derived from a Bas Congo virus (BASV-G). In some embodiments, the BASV-G comprises the amino acid sequence of SEQ ID NO: 73 or 102, or a functional fragment or derivative thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity with SEQ ID NO: 73 or 102. In certain embodiments, the nucleotide sequence that encodes the BASV-G comprises the nucleotide sequence that encodes the amino acid sequence of SEQ ID NO: 73 or 102, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 73 or 102. In certain embodiments, the BASV-G comprises the amino acid sequence of SEQ ID NO: 73 or 102.

In some embodiments, a recombinant fusogenic protein described herein comprises a rhabdovirus glycoprotein from BASV-G. In some embodiments, the BASV-G comprises the sequence SEQ ID NO: 73. In some embodiments, the BASV-G consists of the sequence SEQ ID NO: 73.

In some embodiments, a recombinant fusogenic protein described herein may comprise a rhabdovirus glycoprotein derived from a Bovine Ephemeral fever virus (BEFV-G). In some embodiments, the BEFV-G comprises the amino acid sequence of SEQ ID NO: 74 or 103, or a functional fragment or derivative thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity with SEQ ID NO: 74 or 103. In certain embodiments, the nucleotide sequence that encodes the BEFV-G comprises the nucleotide sequence that encodes the amino acid sequence of SEQ ID NO: 74 or 103, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 74 or 103. In certain embodiments, the BEFV-G comprises the amino acid sequence of SEQ ID NO: 74 or 103.

In some embodiments, a recombinant fusogenic protein described herein comprises a rhabdovirus glycoprotein from BEFV-G. In some embodiments, the BEFV-G comprises the sequence SEQ ID NO: 74. In some embodiments, the BEFV-G consists of the sequence SEQ ID NO: 74.

In some embodiments, a recombinant fusogenic protein described herein may comprise a rhabdovirus glycoprotein derived from a Curionopolis virus (CURV-G). In some embodiments, the CURV-G comprises the amino acid sequence of SEQ ID NO: 75 or 104, or a functional fragment or derivative thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity with SEQ ID NO: 75 or 104. In certain embodiments, the nucleotide sequence that encodes the CURV-G comprises the nucleotide sequence that encodes the amino acid sequence of SEQ ID NO: 75 or 104, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 75 or 104. In certain embodiments, the CURV-G comprises the amino acid sequence of SEQ ID NO: 75 or 104.

In some embodiments, a recombinant fusogenic protein described herein comprises a rhabdovirus glycoprotein from CURV-G. In some embodiments, the CURV-G comprises the sequence SEQ ID NO: 75. In some embodiments, the CURV-G consists of the sequence SEQ ID NO: 75.

In some embodiments, a recombinant fusogenic protein described herein may comprise a rhabdovirus glycoprotein derived from a Drosophila melanogaster sigmavirus (DMelSV-G). In some embodiments, the DMelSV-G comprises the amino acid sequence of SEQ ID NO: 76 or 105, or a functional fragment or derivative thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity with SEQ ID NO: 76 or 105. In certain embodiments, the nucleotide sequence that encodes the DMelSV-G comprises the nucleotide sequence that encodes the amino acid sequence of SEQ ID NO: 76 or 105, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 76 or 105. In certain embodiments, the DMelSV-G comprises the amino acid sequence of SEQ ID NO: 76 or 105.

In some embodiments, a recombinant fusogenic protein described herein comprises a rhabdovirus glycoprotein from DMelSV-G. In some embodiments, the DMelSV-G comprises the sequence SEQ ID NO: 76. In some embodiments, the DMelSV-G consists of the sequence SEQ ID NO: 76.

In some embodiments, a recombinant fusogenic protein described herein may comprise a rhabdovirus glycoprotein derived from a Niakha virus (NIAV-G). In some embodiments, the NIAV-G comprises the amino acid sequence of SEQ ID NO: 77 or 106, or a functional fragment or derivative thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity with SEQ ID NO: 77 or 106. In certain embodiments, the nucleotide sequence that encodes the NIAV-G comprises the nucleotide sequence that encodes the amino acid sequence of SEQ ID NO: 77 or 106, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 77 or 106. In certain embodiments, the NIAV-G comprises the amino acid sequence of SEQ ID NO: 77 or 106.

In some embodiments, a recombinant fusogenic protein described herein comprises a rhabdovirus glycoprotein from NIAV-G. In some embodiments, the NIAV-G comprises the sequence SEQ ID NO: 77. In some embodiments, the NIAV-G consists of the sequence SEQ ID NO: 77.

In some embodiments, a recombinant fusogenic protein described herein may comprise a rhabdovirus glycoprotein derived from a Puerto almandras virus (PTAMV-G). In some embodiments, the PTAMV-G comprises the amino acid sequence of SEQ ID NO: 78 or 107, or a functional fragment or derivative thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity with SEQ ID NO: 78 or 107. In certain embodiments, the nucleotide sequence that encodes the PTAMV-G comprises the nucleotide sequence that encodes the amino acid sequence of SEQ ID NO: 78 or 107, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 78 or 107. In certain embodiments, the PTAMV-G comprises the amino acid sequence of SEQ ID NO: 78 or 107.

In some embodiments, a recombinant fusogenic protein described herein comprises a rhabdovirus glycoprotein from PTAMV-G. In some embodiments, the PTAMV-G comprises the sequence SEQ ID NO: 78. In some embodiments, the PTAMV-G consists of the sequence SEQ ID NO: 78.

In some embodiments, a recombinant fusogenic protein described herein may comprise a rhabdovirus glycoprotein derived from a Tupaia virus (TUPTV-G). In some embodiments, the TUPTV-G comprises the amino acid sequence of SEQ ID NO: 79 or 108, or a functional fragment or derivative thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity with SEQ ID NO: 79 or 108. In certain embodiments, the nucleotide sequence that encodes the TUPTV-G comprises the nucleotide sequence that encodes the amino acid sequence of SEQ ID NO: 79 or 108, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 79 or 108. In certain embodiments, the TUPTV-G comprises the amino acid sequence of SEQ ID NO: 79 or 108.

In some embodiments, a recombinant fusogenic protein described herein comprises a rhabdovirus glycoprotein from TUPTV-G. In some embodiments, the TUPTV-G comprises the sequence SEQ ID NO: 79. In some embodiments, the TUPTV-G consists of the sequence SEQ ID NO: 79.

In some embodiments, a recombinant fusogenic protein described herein may comprise a rhabdovirus glycoprotein described herein, and the rhabdovirus glycoprotein may be truncated, e.g., at the N-terminal and/or the C-terminal end(s), thereby resulting in the production of a truncated rhabdovirus glycoprotein, e.g., a truncated functional fragment, which can retain the ability to impart at least the activity of the rhabdovirus glycoprotein.

In some embodiments, a recombinant fusogenic protein described herein can be a fragment of a rhabdovirus glycoprotein described herein, and the cytoplasmic tail of the rhabdovirus glycoprotein has been removed or truncated, and/or optionally replaced with another sequence.

In some embodiments, a recombinant fusogenic protein described herein can be a fragment of a rhabdovirus glycoprotein described herein, and the fragment of the rhabdovirus glycoprotein may comprise, without limitation, a truncated cytoplasmic tail. As a non-limiting example, the cytoplasmic tail of the rhabdovirus glycoprotein may be truncated by 2 to 80, 3 to 75, 4 to 70, 5 to 65, 6 to 60, 7 to 55, 8 to 50, 9 to 45, 10 to 40, 11 to 35, 12 to 30, 13 to 25, 14 to 20, or 15 to 35 amino acids from the C-terminus. In certain embodiments, the cytoplasmic tail of the rhabdovirus glycoprotein which has been truncated may be truncated 10 to 40 amino acids from the C-terminus. In some embodiments, the cytoplasmic tail of the rhabdovirus glycoprotein may be truncated by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80, or more, amino acids from the C-terminus. In some embodiments, the cytoplasmic tail of the rhabdovirus glycoprotein may be truncated by 30 amino acids from the C-terminus.

In some embodiments, the cytoplasmic tail of a rhabdovirus glycoprotein described herein can be truncated by up to 40 amino acids from the C-terminus. In some embodiments, the cytoplasmic tail of the rhabdovirus glycoprotein can be truncated by 10 to 40 amino acids from the C-terminus. In some embodiments, the cytoplasmic tail of the rhabdovirus glycoprotein can be truncated by 30 amino acids from the C-terminus.

In some embodiments, a recombinant fusogenic protein described herein may further comprise a cytoplasmic tail from VSV-G, or a functional fragment or derivative thereof. A non-limiting example of a cytoplasmic tail is the amino acid sequence of CIKLKHTKKRQIYTDIEMNRLGK (SEQ ID NO: 16). In some embodiments, the cytoplasmic tail of a rhabdovirus glycoprotein described herein comprises the amino acid sequence of SEQ ID NO: 16, or a functional fragment or derivative thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity with SEQ ID NO: 16. In certain embodiments, the nucleotide sequence that encodes the cytoplasmic tail of the rhabdovirus glycoprotein comprises the nucleotide sequence that encodes the amino acid sequence of SEQ ID NO: 16, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 16. In certain embodiments, the cytoplasmic tail of the rhabdovirus glycoprotein comprises the amino acid sequence of SEQ ID NO: 16.

In some embodiments, fusogenic proteins to be included in recombinant viruses of the present disclosure may also include other fusogens. For example, a form of hemagglutinin (HA) from influenza A/fowl plague virus/Rostock/34 (FPV), a class I fusogen, may be used. In some embodiments, a form of FPV HA may be used. HA-mediated fusion is generally independent of receptor binding. As another example, the Sindbis virus glycoprotein (a class II fusogen) from the alphavirus family may be used.

In some embodiments, the fusogenic molecule is a Sindbis virus envelope protein (SIN). The SINdbis virus transfers the SINdbis virus RNA into the cell by low pH mediated membrane fusion. SIN contains five structural proteins, E1, E2, E3, 6K and capsid. E2 contains the receptor binding sequence that allows the wild-type SIN to bind, while E1 is known to contain the properties necessary for membrane fusion. E1, E2, and E3 are encoded by a polyprotein, the amino acid sequence of which is provided, e.g., by Accession No. VHWVB, VHWVB2, and P03316: the nucleic acid sequence is provided, e.g., by Accession No. SVU90536 and V01403.

In some embodiments, the Sindbis virus envelope protein is mutated (SINmu). In certain embodiments, the mutation reduces the natural tropism of the Sindbis virus. In certain embodiments, a SINmu comprising SIN proteins E1, E2, and E3, wherein at least one of E1, E2, or E3 is mutated as compared to a wild-type sequence. For example, one or more of the E1, E2, or E3 proteins can be mutated at one or more amino acid positions. In addition, combinations of mutations in E1, E2, and E3 are encompassed by fusogen as described herein, e.g., mutations in E1 and E2, or in E2 and E3, or E3 and E1, or E1, E2, and E3. In certain embodiments, at least E2 is mutated.

In some embodiments, the SINmu comprises the following envelope protein mutations in comparison to wild-type Sindbis virus envelope proteins, e.g., deletion of E3 amino acids 61-64; (ii) E2 KE159-160AA; and (iii) E2 SLKQ68-71AAAA. In some embodiments, the SINmu comprises the envelope protein mutation E1 AK226-227SG.

Other Togaviridae family envelopes, e.g., from the Alphavirus genus, e.g., Semliki Forest Virus, Ross River Virus, and equine encephalitis virus, can also be used to pseudotype the vectors described herein. The envelope protein sequences for such Alphaviruses are known in the art.

### Linkers

In certain aspects, the present disclosure provides a recombinant fusogenic protein comprising a rhabdovirus glycoprotein (G), or a functional fragment or derivative thereof, and a targeting molecule which can be attached to the N-terminus of the rhabdovirus glycoprotein, or the functional fragment or derivative thereof, via a linker. In some embodiments, the linker can be sensitive to a proteolytic cleavage, for example, via a naturally occurring cell-associated protease. In some embodiments, the linker can be sensitive to a proteolytic cleavage, for example, via an endogenous protease. In some embodiments, the linker can be sensitive to a proteolytic cleavage, for example, via an exogenously added protease. In some embodiments, the linker comprises, for example, without limitation, an Arginine (R) and/or a Lysine (K) residue.

In some embodiments, a linker of the present disclosure may not be sensitive to a proteolytic cleavage by an endogenous protease. In some embodiments, a linker of the present disclosure may not be sensitive to a proteolytic cleavage by an exogenously added protease.

In some embodiments, the linker may be between 1-50 amino acids long. As a non-limiting example, the linker may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, or more, amino acids long. In some embodiments, the linker may be between 1-5, 1-10, 1-15, 1-20, 1-25, 1-30, 1-35, 1-40, or 1-50, or more, amino acids long.

In some embodiments, the linker may be optimized such that the linker does not impose any constraints on the conformation and/or interactions of the linked partners.

In some embodiments, the linkers can be flexible linkers. Suitable linkers can be readily selected and can be of any of a suitable of different lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids. Example flexible linkers include glycine polymers (G)ₙ, glycine-serine polymers (GS)ₙ, where n is an integer of at least one (e.g., from 1-20), glycine-alanine polymers, alanine-serine polymers, and other flexible linkers known in the art.

In some embodiments, the linker may comprise the sequence (GGGS)ₙ (SEQ ID NO: 42), wherein n=1-10, or n is 1, 2, 3, 4, 5, 6, 7, 8, 9, or 10.

In some embodiments, the linker may comprise the sequence (GGGGS)ₙ (SEQ ID NO: 43), wherein n=1-10, or n is 1, 2, 3, 4, 5, 6, 7, 8, 9, or 10.

In some embodiments, the linkers can be rigid linkers.

In some embodiments, linkers of the present disclosure may comprise any linkers set forth in Table 2, or a variant thereof having at least 50, at least 55, at least 60, at least 65, at least 70, at least 75, at least 80, at least 85, at least 90, at least 95, at least 96, at least 97, at least 98 or at least 9900, sequence identity with the linker sequences set forth in Table 2. The linker sequences are shown in underlined text.

TABLE 2
Examples of Linkers

| Name | Sequence | SEQ ID | Cleavability | Notes |
|---|---|---|---|---|
| 20aaL(F) | KRAAASGGS(G₄S)₂GPK | SEQ ID NO: 174 | cleavable | Underlined R is |
| | | | | within the physical |
| | | | | linker, not in the |
| | | | | flanking scFV or |
| | | | | VSV |
| 18aaL(F) | KRAAASGGS(G₄S)₂ | SEQ ID NO: 2 | cleavable | Underlined R is |
| | | | | within the physical |
| | | | | linker, not in the |
| | | | | flanking scFV or |
| | | | | VSV |
| 17aaL(F) | AAASGGS(G₄S)₂ | SEQ ID NO: 36 | non- | — |
| | | | cleavable | |
| 15aaL | (G₄S)₃ | SEQ ID NO: 37 | non- | — |
| (F-m) | | | cleavable | |
| 15aaL(R) | (EAAAK)₃ | SEQ ID NO: 3 | cleavable | — |
| 16aaL(R) | KR(EAAAK)₃ | SEQ ID NO: 4 | cleavable | Underlined R is |
| | | | | within the physical |
| | | | | linker, not in the |
| | | | | flanking scFV or |
| | | | | VSV |
| — | AAARGSPK(G₄S)₃ | SEQ ID NO: 5 | cleavable | — |
| 24aaL(F) | RAAARGSPK(G₄S)₃ | SEQ ID NO: 169 | cleavable | — |
| — | AAARGSPK(G₄S)₃K | SEQ ID NO: 19 | cleavable | Underlined all |
| | | | | residues but K, as |
| | | | | underlined residues |
| | | | | are within the |
| | | | | physical linker and |
| | | | | K is from the |
| | | | | flanking peptide |
| — | K(G₄S)₃ | SEQ ID NO: 20 | cleavable | Underlined all |
| | | | | residues but K, as |
| | | | | underlined residues |
| | | | | are within the |
| | | | | physical linker and |
| | | | | K is from the |
| | | | | flanking peptide |
| — | KR(G₄S)₃ | SEQ ID NO: 21 | cleavable | Underlined all |
| | | | | residues but K, as |
| | | | | underlined residues |
| | | | | are within the |
| | | | | physical linker and |
| | | | | K is from the |
| | | | | flanking peptide |
| — | (G₄S)₃GPK | SEQ ID NO: 6 | cleavable | Underlined all |
| | | | | residues but K, as |
| | | | | underlined residues |
| | | | | are within the |
| | | | | physical linker and |
| | | | | K is from the |
| | | | | flanking peptide |
| — | AAA(G₄S)₃K | SEQ ID NO: 7 | cleavable | Underlined all |
| | | | | residues but K, as |
| | | | | underlined residues |
| | | | | are within the |
| | | | | physical linker and |
| | | | | K is from the |
| | | | | flanking peptide |
| 19aaL | RAAA(G4S)3 | SEQ ID NO: 170 | — | — |

Further non-limiting examples of linkers that may be used include any of various linker sequences set forth in Table 5, or a variant thereof having at least 50, at least 55, at least 60, at least 65, at least 70, at least 75, at least 80, at least 85, at least 90, at least 95, at least 96, at least 97, at least 98 or at least 9900, sequence identity with the linker sequences set forth in Table 5.

In some embodiments, a linker described herein may comprise a linker sequence set forth in the amino acid sequences of SEQ ID NO: 1-7, 18-57, or 164-174 or a variant thereof having at least at least 50, at least 55, at least 60, at least 65, at least 70, at least 75, at least 80, at least 85, at least 90, at least 95, at least 96, at least 97, at least 98 or at least 990%, sequence identity with the linker sequences set forth in the amino acid sequences of SEQ ID NO: 1-7, 18-57, or 164-174.

In some embodiments, a linker sequence described herein may be comprised within an amino acid sequence comprising one or more amino acids of an antibody sequence or antigen-binding fragment thereof described herein. As a non-limiting example, the antigen-binding fragment can be any of various antigen-binding fragments described herein such as, but not limited to, a single-chain fragment variable (scFv) described herein.

In some embodiments, a linker sequence and/or the amino acid sequence comprising the linker sequence described herein may comprise one or more of an amino acid comprising the amino acid sequence EIKR (SEQ ID NO: 58). In some embodiments, a linker sequence and/or the amino acid sequence comprising the linker sequence described herein may comprise the amino acid sequence EIK of an antibody or antigen-binding fragment thereof described herein. In some embodiments, a linker sequence and/or the amino acid sequence comprising the linker sequence described herein may comprise the amino acid sequence EI of an antibody or antigen-binding fragment thereof described herein. In some embodiments, a linker sequence and/or the amino acid sequence comprising the linker sequence described herein may comprise any one of amino acids E, I, K, R, or any combination thereof, of an antibody or antigen-binding fragment thereof described herein.

In some embodiments, a linker sequence and/or the amino acid sequence comprising the linker sequence described herein may comprise one or more of an amino acid comprising the amino acid sequence LGAK (SEQ ID NO: 59). In some embodiments, a linker sequence and/or the amino acid sequence comprising the linker sequence described herein may comprise the sequence LGAK (SEQ ID NO: 59) of an antibody or antigen-binding fragment thereof described herein. In some embodiments, a linker sequence and/or the amino acid sequence comprising the linker sequence described herein may comprise the amino acid sequence LG of an antibody or antigen-binding fragment thereof described herein. In some embodiments, a linker sequence and/or the amino acid sequence comprising the linker sequence described herein may comprise the amino acid L or G, or any combination thereof, of an antibody or antigen-binding fragment thereof described herein.

In some embodiments, a linker sequence described herein may be comprised within an amino acid sequence comprising one or more amino acids of a starting amino acid sequence of a VSV-G protein, or a fragment or derivative thereof, described herein. In some embodiments, a linker sequence and/or the amino acid sequence comprising the linker sequence described herein may comprise one or more of an amino acid comprising the amino acid sequence KFT of a VSV-G protein. In some embodiments, a linker sequence and/or the amino acid sequence comprising the linker sequence described herein may comprise one or more of an amino acid comprising the amino acid sequence FT of a VSV-G protein. In some embodiments, a linker sequence and/or the amino acid sequence comprising the linker sequence described herein may comprise any one amino acids K, F, or T, or any combination thereof, of a VSV-G protein.

In some embodiments, a linker described herein can be a cleavable linker or a non-cleavable linker. In some embodiments, the linker is a cleavable linker. In other embodiments, the linker is a non-cleavable linker. A cleavable linker may be a protease-sensitive linker, a pH-sensitive linker, or a glutathione-sensitive linker. These linkers are generally cleavable only intracellularly and are preferably stable in extracellular environments.

Protease-sensitive linkers are cleavable by protease enzymatic activity. These linkers typically comprise peptide sequences and may be 2-10 amino acids, about 2-5 amino acids, about 5-10 amino acids, about 10 amino acids, about 5 amino acids, about 3 amino acids, or about 2 amino acids in length. In some embodiments, a peptide sequence may comprise naturally-occurring amino acids, e.g., cysteine, alanine, or non-naturally-occurring or modified amino acids. Non-naturally occurring amino acids include 3-amino acids, homo-amino acids, proline derivatives, 3-substituted alanine derivatives, linear core amino acids, N-methyl amino acids, and others known in the art. In some embodiments, a protease-sensitive linker comprises a valine-citrulline or alanine-citrulline dipeptide sequence. In some embodiments, a protease-sensitive linker can be cleaved by a lysosomal protease, e.g., cathepsin B, and/or an endosomal protease.

A pH-sensitive linker is a covalent linkage that readily degrades in high or low pH environments. In some embodiments, a pH-sensitive linker may be cleaved at a pH in a range of 4 to 6. In some embodiments, a pH-sensitive linker comprises a hydrazone or cyclic acetal. In some embodiments, a pH-sensitive linker is cleaved within an endosome or a lysosome.

In some embodiments, a glutathione-sensitive linker comprises a disulfide moiety. In some embodiments, a glutathione-sensitive linker is cleaved by a disulfide exchange reaction with a glutathione species inside a cell. In some embodiments, the disulfide moiety further comprises at least one amino acid, e.g., a cysteine residue.

Non-limiting examples of cleavable linkers are set forth in the amino acid sequences of SEQ ID NO: 1-7, 18-26, 30, 169, 174. In some embodiments, a cleavable linker may comprise SEQ ID NO: 1-7, 18-26, 30, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 1-7, 18-26, 30, 169, 174. In some embodiments, the cleavable linker may be sensitive to cleavage via proteolytic cleavage. In some embodiments, the proteolytic cleavage may occur by way of a naturally occurring cell-associated protease (e.g., an endogenous protease) and/or by way of an exogenously added protease. In some embodiments, the proteolytic cleavage may occur by way of an endogenous protease. In some embodiments, the proteolytic cleavage may occur by way of an exogenous protease.

In some embodiments, a protease capable of proteolytic cleavage, e.g., proteolytic cleavage of a linker described herein, can belong to a class of proteases such as but not limited to serine proteases, cystine proteases or metalloproteinases. In some embodiments, endogenous proteases may be found, for example, without limitation, in the ER, Golgi, at the cell surface of the cell that can release a virus, in the supernatant (or body fluids), at the surface of the cell that can be targeted by the virus, or in the endocytic compartment of the target cell. Protease cleavage signals for various proteases have been extensively studied using, e.g., high throughput proteomic approaches known in the art. A skilled artisan will understand that there are a great many cleavage signal options for a given protease which can depend to a large extent on context. In some embodiments, protease-cleavage signal pairings can have differing kinetics of cleavage and many membrane proteins may be only partially shed from the cell surface, e.g., due slower kinetics and/or regulated activity of their cleaving proteases (e.g., sheddases). In some embodiments, a protease described herein may comprise any of various proteases, for example, as described in Encyclopedia of Cell Biology. 2016:650-60; Int. J. Mol. Sci. 2020, 21(18), 6805 and/or Molecular Neurobiology volume 56, pages 3090-3112, 2019, both of which are incorporated herein by reference in their entirety for all purposes, or variants thereof.

In some embodiments, the linker can be a non-cleavable linker (also known as an uncleavable linker). Generally, a non-cleavable linker cannot be readily degraded in a cellular or physiological environment. Non-limiting examples of non-cleavable linkers are set forth in the amino acid sequences of SEQ ID NO: 27-29, 31, 32, 36, 37. In some embodiments, a cleavable linker may comprise SEQ ID NO: 27-29, 31, 32, 36, 37, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 27-29, 31, 32.

In various embodiments, a linker described herein can be comprised within a sequence comprising, for example, without limitation, AAASGGS(G₄S)₂GPK (SEQ ID NO: 1); KRAAASGGS(G₄S)₂GPK (SEQ ID NO: 174), KRAAASGGS(G₄S)₂ (SEQ ID NO: 2); (EAAAK)₃ (SEQ ID NO: 3); KR(EAAAK)₃ (SEQ ID NO: 4); AAARGSPK(G₄S)₃ (SEQ ID NO: 5); KRAAARGSPK(G₄S)₃ (SEQ ID NO: 18); RAAARGSPK(G₄S)₃ (SEQ ID NO: 169); AAARGSPK(G₄S)₃K (SEQ ID NO: 19); K(G₄S)₃ (SEQ ID NO: 20); KR(G₄S)₃ (SEQ ID NO: 21); (G₄S)₃GPK (SEQ ID NO: 6); or AAA(G₄S)₃K (SEQ ID NO: 7).

In some embodiments, the linker described herein can be comprised within a sequence set forth in SEQ ID NO: 1-7, 18-57, or 164-174 or a variant thereof, or a sequence having at least 50, at least 55, at least 60, at least 65, at least 70, at least 75, at least 80, at least 85, at least 90, at least 95, at least 96, at least 97, at least 98 or at least 99%, sequence identity with the sequences set forth in SEQ ID NO: 1-7, 18-57, or 164-174.

In some embodiments, the linker described herein can be comprised within a sequence set forth in Table 5, or a variant thereof, or a sequence having at least 50, at least 55, at least 60, at least 65, at least 70, at least 75, at least 80, at least 85, at least 90, at least 95, at least 96, at least 97, at least 98 or at least 99%, sequence identity with the sequences set for in Table 5.

In some embodiments, the linker described herein can be comprised within a sequence set forth in Table 2, or a variant thereof, or a sequence having at least 50, at least 55, at least 60, at least 65, at least 70, at least 75, at least 80, at least 85, at least 90, at least 95, at least 96, at least 97, at least 98 or at least 99%, sequence identity with the sequences set for in Table 2.

In some embodiments, a linker sequence and/or the amino acid sequence comprising the linker sequence described herein described herein may comprise the amino acid sequence alanine-alanine-alanine (AAA) which may be replaced with the amino acid sequence glycine-glycine-glycine (GGG) (i.e., the linker and/or the amino acid sequence comprising the linker may comprise AAA to GGG mutations) which can, e.g., allow for more freedom of movement of a displayed domain (e.g., a targeting molecule described herein) on a linker described herein.

While both alanine (A) and glycine (G) are the simplest non-polar neutral amino acids, glycine (G) is hydrophilic, and alanine (A) is hydrophobic. In some embodiments, a linker described herein can be located in between the N-terminus of a VSV-G (e.g., a blinded VSV-G), and a displayed domain (e.g., a targeting molecule described herein) which can, e.g., attach to a receptor on a desired cell. Therefore, since the linker would be exposed to the external environment, placing a hydrophilic amino acid (e.g., glycine (G)) in the linker sequence may allow for more flexibility and movement of the displayed domain which can enhance targeting of the displayed domain. A hydrophobic amino acid (e.g., alanine (A)) may affect the tertiary conformation of a protein in the external hydrophilic environment and drive the linker, in addition to the displayed domain, in on itself. This could reduce the movement capacity of the displayed domain for accessing the desired receptor of the displayed domain. In some embodiments, a hydrophilic amino acid (glycine (G)) may allow engineering of a virus particle described herein to be easier, e.g., if folding was impaired by the presence of a hydrophobic amino acid (e.g., alanine (A))

As a non-limiting example, a linker sequence described herein may comprise the amino acid sequence RAAASGGS(G₄S)₂ (SEQ ID NO: 172) which when mutated to comprise AAA to GGG mutations described herein may comprise the amino acid sequence RGGGSGGS(G₄S)₂ (SEQ ID NO: 176). As another non-limiting example, an amino acid sequence comprising a linker sequence may comprise the amino acid sequence KRAAASGGS(G₄S)₂ (SEQ ID NO: 2) which when mutated to comprise AAA to GGG mutations described herein may comprise the amino acid sequence KRGGGSGGS(G₄S)₂ (SEQ ID NO: 175). In some embodiments, a linker sequence described herein may comprise the amino acid sequence RGGGSGGS(G₄S)₂ (SEQ ID NO: 176). In some embodiments, an amino acid sequence comprising a linker sequence described herein may comprise the amino acid sequence KRGGGSGGS(G₄S)₂ (SEQ ID NO: 175).

As yet another non-limiting example a linker sequence described herein may comprise the amino acid sequence RAAASGGS(G₄S)₂GP (SEQ ID NO: 171) which when mutated to comprise AAA to GGG mutations described herein may comprise the amino acid sequence RGGGSGGS(G₄S)₂GP (SEQ ID NO: 178). As still yet another non-limiting example, an amino acid sequence comprising a linker sequence may comprise the amino acid sequence KRAAASGGS(G₄S)₂GPK (SEQ ID NO: 174) which when mutated to comprise AAA to GGG mutations described herein may comprise the amino acid sequence KRGGGSGGS(G₄S)₂GPK (SEQ ID NO: 177). In some embodiments, a linker sequence described herein may comprise the amino acid sequence RGGGSGGS(G₄S)₂GP (SEQ ID NO: 178). In some embodiments, an amino acid sequence comprising a linker sequence described herein may comprise the amino acid sequence KRGGGSGGS(G₄S)₂GPK (SEQ ID NO: 177).

In some embodiments, a linker sequence described herein may comprise the amino acid sequence (EAAAK)₃ (SEQ ID NO: 3) which when mutated to comprise AAA to GGG mutations described herein may comprise the amino acid sequence (EGGGK)₃ (SEQ ID NO: 205). In some embodiments, a linker sequence described herein may comprise the amino acid sequence (EGGGK)₃ (SEQ ID NO: 205).

In some embodiments, a linker sequence described herein may comprise the amino acid sequence R(EAAAK)₃ (SEQ ID NO: 173) which when mutated to comprise AAA to GGG mutations described herein may comprise the amino acid sequence R(EGGGK)₃ (SEQ ID NO: 207). In some embodiments, an amino acid sequence comprising a linker sequence may comprise the amino acid sequence KR(EAAAK)₃ (SEQ ID NO: 4) which when mutated to comprise AAA to GGG mutations described herein may comprise the amino acid sequence KR(EGGGK)₃ (SEQ ID NO: 206). In some embodiments, a linker sequence described herein may comprise the amino acid sequence R(EGGGK)₃ (SEQ ID NO: 207). In some embodiments, an amino acid sequence comprising a linker sequence described herein may comprise the amino acid sequence KR(EGGGK)₃ (SEQ ID NO: 206).

In some embodiments, a linker sequence described herein may comprise the amino acid sequence AAARGSPK(G₄S)₃ (SEQ ID NO: 5) which when mutated to comprise AAA to GGG mutations described herein may comprise the amino acid sequence GGGRGSPK(G₄S)₃ (SEQ ID NO: 208). In some embodiments, a linker sequence described herein may comprise the amino acid sequence GGGRGSPK(G₄S)₃ (SEQ ID NO: 208).

In some embodiments, a linker sequence described herein may comprise the amino acid sequence RAAARGSPK(G₄S)₃ (SEQ ID NO: 169) which when mutated to comprise AAA to GGG mutations described herein may comprise the amino acid sequence RGGGRGSPK(G₄S)₃ (SEQ ID NO: 209). In some embodiments, a linker sequence described herein may comprise the amino acid sequence RGGGRGSPK(G₄S)₃ (SEQ ID NO: 209).

In some embodiments, an amino acid sequence comprising a linker sequence may comprise the amino acid sequence AAARGSPK(G₄S)₃K (SEQ ID NO: 19) which when mutated to comprise AAA to GGG mutations described herein may comprise the amino acid sequence GGGRGSPK(G₄S)₃K (SEQ ID NO: 210). In some embodiments, an amino acid sequence comprising a linker sequence described herein may comprise the amino acid sequence GGGRGSPK(G₄S)₃K (SEQ ID NO: 210).

In some embodiments, a linker sequence described herein may comprise the amino acid sequence AAA(G₄S)₃ (SEQ ID NO: 34) which when mutated to comprise AAA to GGG mutations described herein may comprise the amino acid sequence GGG(G₄S)₃ (SEQ ID NO: 212). In some embodiments, an amino acid sequence comprising a linker sequence may comprise the amino acid sequence AAA(G₄S)₃K (SEQ ID NO: 7) which when mutated to comprise AAA to GGG mutations described herein may comprise the amino acid sequence GGG(G₄S)₃K (SEQ ID NO: 211). In some embodiments, a linker sequence described herein may comprise the amino acid sequence GGG(G₄S)₃ (SEQ ID NO: 212). In some embodiments, an amino acid sequence comprising a linker sequence described herein may comprise the amino acid sequence GGG(G₄S)₃K (SEQ ID NO: 211).

### Targeting Molecules

In various embodiments, a recombinant fusogenic protein described herein may comprise a rhabdovirus glycoprotein (G), or a functional fragment or derivative thereof and a targeting molecule. The targeting molecule may be located at the N-terminus of the rhabdovirus glycoprotein. In certain aspects, the targeting molecule may be attached to the N-terminus of the rhabdovirus glycoprotein, or the functional fragment or derivative thereof, e.g., by way of any of various linkers, or combinations thereof, described herein. In some embodiments, the N-terminus of the rhabdovirus glycoprotein, or the functional fragment or derivative thereof, to which the targeting molecule is attached, via a linker, does not comprise one or more amino acids present at the N-terminus of a mature wild-type rhabdovirus glycoprotein. In some embodiments, the rhabdovirus glycoprotein (G) which is attached to the targeting molecule may comprise any of various rhabdovirus glycoproteins, or functional fragments or derivatives thereof, described herein. For example, as described herein, the rhabdovirus glycoprotein may be derived from a vesicular stomatitis virus glycoprotein (VSV-G), a Flanders virus glycoprotein (FLAV-G), a Chandipura virus glycoprotein (CHPV-G), a Perinet virus glycoprotein (PERV-G), a Piry virus glycoprotein (PIRYV-G), a Fukuoka virus glycoprotein (FUKV-G), a Joinjakaka virus glycoprotein (JOIV-G), a Kumasi virus glycoprotein (KRV-G), a Isfahan glycoprotein (ISFV-G), a Jurona glycoprotein (JURV-G), a Mediterranean Bat glycoprotein (MBV-G), a Malpais Spring glycoprotein (MSPV-G), a Radi glycoprotein (RADV-G), a Rhinolophus affinis-G, a Yug Bugdanavoc glycoprotein (YBV-G), a Yinshui Bat glycoprotein (YSBV-G), a Keuraliba glycoprotein (KEUV-G), a Kimberley glycoprotein (KIMV-G), a Kanyawara glycoprotein (KYAV-G), a La Joya glycoprotein (LJV-G), a Mosquiero glycoprotein (MQOV-G), a Parry Creek glycoprotein (PCV-G), a Bas Congo glycoprotein (BASV-G), a Bovine Ephemeral fever glycoprotein (BEFV-G), a Curionopolis glycoprotein (CURV-G), a Drosophila melanogaster sigmavirus glycoprotein (DMelSV-G), a Niakha glycoprotein (NIAV-G), a Puerto almandras glycoprotein (PTAMV-G), or a Tupaia rhabdovirus (TUPTV-G), or a functional fragment or derivative thereof.

In some embodiments, when the rhabdovirus G protein comprises a VSV-G, or a functional fragment or derivative thereof, the targeting molecule can be capable of interfering with the ability of the VSV-G, or the functional fragment or derivative thereof, to interact with low-density lipoprotein receptor (LDLR). In some embodiments, the targeting molecule can interfere with the ability of the VSV-G, or the functional fragment or derivative thereof, to interact with LDLR, for example, by way of steric hindrance.

Non-limiting examples of a targeting molecule include an antibody or antigen-binding fragment thereof, an affibody, a darpin, a peptide, a natural or modified natural receptor ligand, a T cell receptor (TCR) or fragment or derivative thereof, or an MHC-peptide complex or a fragment or derivative thereof.

In some embodiments, the targeting molecule targets, e.g., epidermal growth factor receptor (EGFR), human epidermal growth factor receptor 2 (HER2), mucin 16 (MUC16), cKit, alpha-v beta-3 (αVβ3) Integrin, insulin like growth factor 1 receptor (IGF1R), B-cell maturation antigen (BCMA), Nectin-4, mitogen-activated protein kinase kinase (MEK), cluster of differentiation 44 (CD44), CD3, CD4, CD28, stem cell factor (SCF), thrombopoietin, c-Met, CXCR4, IL2R, or interleukin 3 (IL-3).

In some embodiments, the targeting molecule described herein targets a cell, e.g., a cancer cell such as but not limited to a tumor cell. Examples of cancer cells include cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma, trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; sertoli cell carcinoma; Leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; Mullerian mixed tumor; nephroblastoma; hepatoblastoma; carcinosarcoma; mesenchymoma, malignant; Brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; Kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma, paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma, mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some embodiments, the targeting molecule is selected from a humanized antibody or antigen binding fragment thereof, human antibody or antigen binding fragment thereof, murine antibody or antigen binding fragment thereof, chimeric antibody or antigen binding fragment thereof, monovalent Fab′, divalent Fab2, F(ab)′3 fragments, single-chain fragment variable (scFv), bis-scFv, (scFv)2, diabody, minibody, nanobody, triabody, tetrabody, disulfide stabilized Fv protein (dsFv), single-domain antibody (sdAb), Ig NAR, single heavy chain antibody, bispecific antibody or biding fragment thereof, bi-specific T-cell engager (BiTE), trispecific antibody, or chemically modified derivatives thereof.

An antibody described herein can comprise immunoglobulin molecules comprising four polypeptide chains, two heavy chains (HCs) and two light chains (LCs), inter-connected by disulfide bonds (e.g., IgG). In various embodiments, each antibody heavy chain (HC) comprises a heavy chain variable region (“HCVR” or “V_{H}”) and a heavy chain constant region; and each antibody light chain (LC) comprises a light chain variable region (“LCVR or “V_{L}”) and a light chain constant region (CL). The V_{H} and V_{L} regions can be further subdivided into regions of hypervariability, termed complementarity determining regions (CDR), interspersed with regions that are more conserved, termed framework regions (FR).

In some embodiments, an antibody or antigen-binding fragment thereof, comprises a heavy chain constant domain, e.g., of the type of IgA (e.g., IgA1 or IgA2), IgD, IgE, IgG (e.g., IgG1, IgG2, IgG3 and IgG4 (e.g., comprising a S228P and/or S108P mutation)) or IgM. In some embodiments, an antibody or antigen-binding fragment thereof, can comprise a light chain constant domain, e.g., of the type of kappa or lambda. In an embodiment of the disclosure, a V_{H} can be linked to a human heavy chain constant domain (e.g., IgG) and a V_{L} can be linked to a human light chain constant domain (e.g., kappa).

In some embodiments, the assignment of amino acids to each framework or CDR domain is in accordance with the definitions of Sequences of Proteins of Immunological Interest, Kabat et al.; National Institutes of Health, Bethesda, Md.; 5th ed.; NIH Publ. No. 91-3242 (1991); Kabat (1978) Adv. Prot. Chem. 32:1-75; Kabat et al., (1977) J. Biol. Chem. 252:6609-6616; Chothia et al., (1987) J Mol. Biol. 196:901-917 or Chothia et al., (1989) Nature 342:878-883. Thus, the present disclosure includes antibodies and antigen-binding fragments including the CDRs of a VH and the CDRs of a VL, which VH and VL comprise amino acid sequences as set forth herein (or a variant thereof), wherein the CDRs are as defined, for example, according to Kabat and/or Chothia.

In some embodiments, an antibody or antigen-binding fragment can comprise a heavy chain constant domain, e.g., of the type of IgA (e.g., IgA1 or IgA2), IgD, IgE, IgG (e.g., IgG1, IgG2, IgG3 and IgG4 (e.g., comprising a S228P and/or S108P mutation)) or IgM. In an embodiment of the disclosure, an antigen-binding protein, e.g., antibody or antigen-binding fragment, comprises a light chain constant domain, e.g., of the type of kappa or lambda.

The term “human” antibody or antigen-binding fragment, as used herein, includes antibodies and fragments having human amino acid sequence; for example, variable and constant regions derived from human germline immunoglobulin sequences whether in a human cell or grafted into a non-human cell, e.g., a mouse cell. The human antibodies and antigen-binding fragments of the disclosure may, in an embodiment of the disclosure, include amino acid residues not encoded by human germline immunoglobulin sequences (e.g., having mutations introduced by random or site-specific mutagenesis in vitro or by somatic mutation in vivo), for example in the CDRs and, in particular, CDR3. However, the term “human antibody”, as used herein, is not intended to include mAbs in which CDR sequences derived from the germline of another mammalian species (e.g., mouse) have been grafted onto human FR sequences. The term includes antibodies recombinantly produced in a non-human mammal or in cells of a non-human mammal. The term is not intended to include antibodies isolated from or generated in a human subject.

The present disclosure includes chimeric antibodies and antigen-binding fragments thereof, and methods of use thereof. As used herein, a “chimeric antibody” is an antibody having the variable domain from a first antibody and the constant domain from a second antibody, where the first and second antibodies are from different species. (see e.g., U.S. Pat. No. 4,816,567; and Morrison et al., (1984) Proc. Natl. Acad. Sci. USA 81: 6851-6855). The present disclosure includes chimeric antibodies comprising the variable domains and a non-human constant domain.

An antigen-binding fragment of an antibody will, in various embodiments, comprise less than a full antibody but still binds specifically to antigen, e.g., including at least one variable domain. The variable domain may be of any size or amino acid composition and will generally comprise at least one (e.g., 3) CDR(s) which is adjacent to or in frame with one or more framework sequences. In antigen-binding fragments having a V_{H} domain associated with a V_{L} domain, the V_{H} and VL domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} and/or V_{L} domain which are bound non-covalently.

In certain embodiments, an antigen-binding fragment of an antibody may contain at least one variable domain covalently linked to at least one constant domain. Non-limiting, example configurations of variable and constant domains that may be found within an antigen-binding fragment of an antibody of the present disclosure include: (i) V_{H}-CH1; (ii) V_{H}-CH2; (iii) V_{H}-CH3; (iv) V_{H}-CH1-CH2; (v) V_{H}-CH1-CH2-CH3; (vi) V_{H}-CH2-CH3; (vii) V_{H}-CL; (viii) V_{L}-CH1; (ix) V_{L}-CH2; (x) V_{L}-CH3; (xi) V_{L}-CH1-CH2; (xii) V_{L}-CH1-CH2-CH3; (xiii) V_{L}-CH2-CH3; and (xiv) V_{L}-CL. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60, or more) amino acids, which result in a flexible or semi-flexible linkage between adjacent variable and/or constant domains in a single polypeptide molecule. Moreover, an antigen-binding fragment of an antibody of the present disclosure may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

Antibodies and antigen-binding fragments thereof may be monospecific or multispecific (e.g., bispecific).

The antibodies and antigen-binding fragments described herein may be fused to other polypeptide molecules such as, but are not limited to, an epitope (e.g., FLAG) or a tag sequence (e.g., a His tag sequence, and the like) to allow for the detection and/or isolation of the antibody or antigen-binding fragment; a ligand or a portion thereof which binds to a transmembrane receptor protein; an enzyme or portion thereof which is catalytically active; a polypeptide or peptide which promotes oligomerization, such as a leucine zipper domain; a polypeptide or peptide which increases stability, such as an immunoglobulin constant region (e.g., an Fc domain); a half-life extending polypeptide (e.g., albumin or albumin-binding peptides/proteins); a functional or non-functional antibody, or a heavy or light chain thereof, and/or a polypeptide which has an activity different from the antibody or antigen-binding fragment of the present disclosure.

In some embodiments, the antibodies or antigen-binding fragments thereof described herein may be post-translationally modified including, for example: Glu or Gln cyclization at N-terminus; Loss of positive N-terminal charge; Lys variants at C-terminus; Deamidation (Asn to Asp); Isomerization (Asp to isoAsp); Deamidation (Gln to Glu); Oxidation (Cys, His, Met, Tyr, Trp); and/or Disulfide bond heterogeneity (Shuffling, thioether and trisulfide formation).

In some embodiments, the antigen-binding protein comprises a fragment antigen-binding region (Fab). In some embodiments, the antigen-binding protein comprises a single chain fragment variable (scFv). In some embodiments, the scFv comprises domains arranged in the following orientation from N-terminus to C-terminus: HCVR-LCVR. In some embodiments, the scFv comprises domains arranged in the following orientation from N-terminus to C-terminus: LCVR-HCVR. wherein the scFv variable regions are connected by a linker such as, but not limited to, any of various linkers described herein.

The term “specifically binds” or “binds specifically” refers to targeting molecules (e.g., antibodies or antigen-binding fragments thereof) having a binding affinity to an antigen. The present disclosure includes targeting molecules that specifically bind to, for example, without limitation, EGFR, HER2, MUC16, cKit, αVβ3 Integrin, IGF1R, BCMA, Nectin-4, MEK, CD44, CD3, CD4, CD28, stem cell factor, thrombopoietin, c-Met, CXCR4, IL2R, or IL-3. In some embodiments, the targeting molecule disclosed herein can comprise an scFv which targets the tumor antigen HER2 (which can also be called Human Epidermal Growth Factor Receptor 2, HER-2, c-erbB-2, C-ErbB-2, C-ERB-2, c-ERB2, and the like). In some embodiments, the anti-HER2 scFv comprises domains arranged in the following orientation from N-terminus to C-terminus: HCVR-LCVR. In some embodiments, the anti-HER2 scFv comprises domains arranged in the following orientation from C-terminus to N-terminus: LCVR-HCVR. In some embodiments the scFv variable regions of the anti-HER2 scFv are connected by a linker such as but not limited to a linker described herein. In some embodiments, the anti-HER2 scFv specifically binds to human HER2. As an example, an anti-HER2 scFv which can be used in the practice of the present disclosure may comprise any anti-HER2 scFv as described, e.g., by Shier and colleagues (Shier et al., Isolation of picomolar affinity anti-c-erbB-2 single-chain Fv by molecular evolution of the complementarity determining regions in the center of the antibody binding site. J Mol Biol. 1996 Nov. 8; 263(4):551-67, the contents of which are incorporated herein by reference in their entirety), or variants thereof. In some embodiments, the anti-HER2 scFv can comprise a clone C6-B1D2 such as that described by Shier et al. (1996) and can bind to human HER2, in some cases, with a K_{d} of about, e.g., 0.15×10⁻¹⁰ M.

In some embodiments, the anti-HER2 scFv described herein is derived from antibody C6B1D2. In some embodiments, the anti-HER2 scFv derived from antibody C6B1D2 comprises an amino acid sequence of SEQ ID NO: 158, or an amino acid sequence having at least 80% sequence identity thereof. In some embodiments, the nucleotide sequence that encodes anti-HER2 scFv derived from antibody C6B1D2 comprises the nucleotide sequence that encodes the amino acid sequence of SEQ ID NO: 158, or an amino acid sequence having at least 80% identity thereof.

In some embodiments, the anti-HER2 scFv derived from antibody C6B1D2 comprises a heavy chain variable region (HCVR) comprising an amino acid sequence of SEQ ID NO: 159, or an amino acid sequence having at least 80% sequence identity thereof. In some embodiments, the nucleotide sequence that encodes the HCVR of the anti-HER2 scFv derived from antibody C6B1 D2 comprises the nucleotide sequence that encodes the amino acid sequence of SEQ ID NO: 159, or an amino acid sequence having at least 80% identity thereof. In some embodiments, the anti-HER2 scFv derived from antibody C6B1D2 comprises a light chain variable region (LCVR) comprising an amino acid sequence of SEQ ID NO: 160, or an amino acid sequence having at least 80% sequence identity thereof. In some embodiments, the nucleotide sequence that encodes the LCVR of the anti-HER2 scFv derived from antibody C6B1D2 comprises the nucleotide sequence that encodes the amino acid sequence of SEQ ID NO: 160, or an amino acid sequence having at least 80% identity thereof. In some embodiments, the anti-HER2 scFv derived from antibody C6B1D2 comprises a linker sequence between the HCVR and the LCVR, and the linker sequence comprises an amino acid sequence of SEQ ID NO: 37, or an amino acid sequence having at least 80% sequence identity thereof. In some embodiments, the nucleotide sequence that encodes the linker between the HCVR and the LCVR of the anti-HER2 scFv derived from antibody C6B1D2 comprises the nucleotide sequence that encodes the amino acid sequence of SEQ ID NO: 37, or an amino acid sequence having at least 80% identity thereof. In some embodiments, the anti-HER2 scFv derived from the antibody C6B1D2 comprises domains arranged in the following orientation from N-terminus to C-terminus: HCVR-LCVR. In some embodiments, the anti-HER2 scFv derived from the antibody C6B1D2 comprises domains arranged in the following orientation from N-terminus to C-terminus: LCVR-HCVR, and the anti-HER2 scFv variable regions are connected by a linker such as, but not limited to, any of various linkers described herein.

In some embodiments, the anti-HER2 scFv described herein is derived from antibody C6.5. In some embodiments, the anti-HER2 scFv derived from antibody C6.5 comprises an amino acid sequence of SEQ ID NO: 192, or an amino acid sequence having at least 80% sequence identity thereof. In some embodiments, the nucleotide sequence that encodes anti-HER2 scFv derived from antibody C6.5 comprises the nucleotide sequence that encodes the amino acid sequence of SEQ ID NO: 192, or an amino acid sequence having at least 80% identity thereof.

In some embodiments, the anti-HER2 scFv derived from antibody C6.5 comprises a heavy chain variable region (HCVR) comprising an amino acid sequence of SEQ ID NO: 193, or an amino acid sequence having at least 80% sequence identity thereof. In some embodiments, the nucleotide sequence that encodes the HCVR of the anti-HER2 scFv derived from antibody C6.5 comprises the nucleotide sequence that encodes the amino acid sequence of SEQ ID NO: 193, or an amino acid sequence having at least 80% identity thereof. In some embodiments, the anti-HER2 scFv derived from antibody C6.5 comprises a light chain variable region (LCVR) comprising an amino acid sequence of SEQ ID NO: 194, or an amino acid sequence having at least 80% sequence identity thereof. In some embodiments, the nucleotide sequence that encodes the LCVR of the anti-HER2 scFv derived from antibody C6.5 comprises the nucleotide sequence that encodes the amino acid sequence of SEQ ID NO: 194, or an amino acid sequence having at least 80% identity thereof. In some embodiments, the anti-HER2 scFv derived from antibody C6.5 comprises a linker sequence between the HCVR and the LCVR, and the linker sequence comprises an amino acid sequence of SEQ ID NO: 55, or an amino acid sequence having at least 80% sequence identity thereof. In some embodiments, the nucleotide sequence that encodes the linker between the HCVR and the LCVR of the anti-HER2 scFv derived from antibody C6.5 comprises the nucleotide sequence that encodes the amino acid sequence of SEQ ID NO: 55, or an amino acid sequence having at least 80% identity thereof. In some embodiments, the anti-HER2 scFv derived from the antibody C6.5 comprises domains arranged in the following orientation from N-terminus to C-terminus: HCVR-LCVR. In some embodiments, the anti-HER2 scFv derived from the antibody C6.5 comprises domains arranged in the following orientation from N-terminus to C-terminus: LCVR-HCVR, and the anti-HER2 scFv variable regions are connected by a linker such as, but not limited to, any of various linkers described herein.

In some embodiments, the targeting molecule disclosed herein can comprise an scFv which targets the tumor antigen EGFR (which can also be called Epidermal Growth Factor Receptor, ERBB1, Receptor Tyrosine-Protein Kinase ErbB-1, and the like). In some embodiments, the anti-EGFR scFv comprises domains arranged in the following orientation from N-terminus to C-terminus: HCVR-LCVR. In some embodiments, the anti-EGFR scFv comprises domains arranged in the following orientation from C-terminus to N-terminus: LCVR-HCVR. In some embodiments the scFv variable regions of the anti-EGFR scFv are connected by a linker such as but not limited to a linker described herein. In some embodiments, the anti-EGFR scFv specifically binds to human EGFR. As an example, an anti-EGFR scFv which can be used in the practice of the present disclosure may comprise any anti-EGFR scFv as described, e.g., by Nakamura and colleagues (Nakamura et al., Antibody-targeted cell fusion, Nature Biotechnology volume 22, pages 331-336 (2004), the contents of which are incorporated herein by reference in their entirety), or variants thereof.

In some embodiments, the anti-EGFR scFv described herein comprises an amino acid sequence of SEQ ID NO: 161, or an amino acid sequence having at least 80% sequence identity thereof. In some embodiments, the nucleotide sequence that encodes the anti-EGFR scFv comprises the nucleotide sequence that encodes the amino acid sequence of SEQ ID NO: 161, or an amino acid sequence having at least 80% identity thereof.

In some embodiments, the anti-EGFR scFv described herein comprises a heavy chain variable region (HCVR) comprising an amino acid sequence of SEQ ID NO: 162, or an amino acid sequence having at least 80% sequence identity thereof. In some embodiments, the nucleotide sequence that encodes the HCVR of the anti-EGFR scFv comprises the nucleotide sequence that encodes the amino acid sequence of SEQ ID NO: 162, or an amino acid sequence having at least 80% identity thereof. In some embodiments, the anti-EGFR scFv comprises a light chain variable region (LCVR) comprising an amino acid sequence of SEQ ID NO: 163, or an amino acid sequence having at least 80% sequence identity thereof. In some embodiments, the nucleotide sequence that encodes the LCVR of the anti-EGFR scFv comprises the nucleotide sequence that encodes the amino acid sequence of SEQ ID NO: 163, or an amino acid sequence having at least 80% identity thereof. In some embodiments, the anti-EGFR scFv comprises a linker sequence between the HCVR and the LCVR, and the linker sequence comprises an amino acid sequence of SEQ ID NO: 37, or an amino acid sequence having at least 80% sequence identity thereof. In some embodiments, the nucleotide sequence that encodes the linker between the HCVR and the LCVR of the anti-EGFR scFv comprises the nucleotide sequence that encodes the amino acid sequence of SEQ ID NO: 37, or an amino acid sequence having at least 80% identity thereof. In some embodiments, the anti-EGFR scFv comprises domains arranged in the following orientation from N-terminus to C-terminus: HCVR-LCVR. In some embodiments, the anti-EGFR scFv comprises domains arranged in the following orientation from N-terminus to C-terminus: LCVR-HCVR, and the anti-EGFR scFv variable regions are connected by a linker such as, but not limited to, any of various linkers described herein.

In some embodiments, the targeting molecule disclosed herein can comprise an scFv which targets the tumor antigen cKit (which can also be called c-kit, KIT Proto-Oncogene, Receptor Tyrosine Kinase, SCFR, V-Kit Hardy-Zuckerman 4 Feline Sarcoma Viral Oncogene Homolog, Mast/Stem Cell Growth Factor Receptor Kit, CD117, PBT, Tyrosine-Protein Kinase Kit, Piebald Trait Protein, Proto-Oncogene C-Kit, P145 C-Kit, C-Kit, V-Kit Hardy-Zuckerman 4 Feline Sarcoma Viral Oncogene-Like Protein, Proto-Oncogene Tyrosine-Protein Kinase Kit, C-Kit Protooncogene, Piebald Trait, CD117 Antigen, MASTC, C-KIT, and the like). In some embodiments, the anti-cKit scFv comprises domains arranged in the following orientation from N-terminus to C-terminus: HCVR-LCVR. In some embodiments, the anti-cKit scFv comprises domains arranged in the following orientation from C-terminus to N-terminus: LCVR-HCVR. In some embodiments the scFv variable regions of the anti-cKit scFv are connected by a linker such as but not limited to a linker described herein. In some embodiments, the anti-cKit scFv specifically binds to human cKit.

In some embodiments, the anti-cKit scFv described herein comprises an amino acid sequence of SEQ ID NO: 195, or an amino acid sequence having at least 80% sequence identity thereof. In some embodiments, the nucleotide sequence that encodes the anti-cKit scFv comprises the nucleotide sequence that encodes the amino acid sequence of SEQ ID NO: 195, or an amino acid sequence having at least 80% identity thereof.

In some embodiments, the anti-cKit scFv described herein comprises a heavy chain variable region (HCVR) comprising an amino acid sequence of SEQ ID NO: 196, or an amino acid sequence having at least 80% sequence identity thereof. In some embodiments, the nucleotide sequence that encodes the HCVR of the anti-cKit scFv comprises the nucleotide sequence that encodes the amino acid sequence of SEQ ID NO: 196, or an amino acid sequence having at least 80% identity thereof. In some embodiments, the anti-cKit scFv comprises a light chain variable region (LCVR) comprising an amino acid sequence of SEQ ID NO: 197, or an amino acid sequence having at least 80% sequence identity thereof. In some embodiments, the nucleotide sequence that encodes the LCVR of the anti-cKit scFv comprises the nucleotide sequence that encodes the amino acid sequence of SEQ ID NO: 197, or an amino acid sequence having at least 80% identity thereof. In some embodiments, the anti-cKit scFv comprises a linker sequence between the HCVR and the LCVR, and the linker sequence comprises an amino acid sequence of SEQ ID NO: 55, or an amino acid sequence having at least 80% sequence identity thereof. In some embodiments, the nucleotide sequence that encodes the linker between the HCVR and the LCVR of the anti-cKit scFv comprises the nucleotide sequence that encodes the amino acid sequence of SEQ ID NO: 55, or an amino acid sequence having at least 80% identity thereof. In some embodiments, the anti-cKit scFv comprises domains arranged in the following orientation from N-terminus to C-terminus: HCVR-LCVR. In some embodiments, the anti-cKit scFv comprises domains arranged in the following orientation from N-terminus to C-terminus: LCVR-HCVR, and the anti-cKit scFv variable regions are connected by a linker such as, but not limited to, any of various linkers described herein.

In some embodiments, the targeting molecule disclosed herein can comprise a nanobody which targets the tumor antigen EGFR. A non-limiting example of a nanobody which targets EGFR is nanobody Nb 7D12 (anti-EGFR) (also called EGFRNb). In some embodiments, the nanobody Nb 7D12 (anti-EGFR) can comprise the amino acid sequence QVKLEESGGGSVQTGGSLRLTCAASGRTSRSYGMGWFRQAPGKEREFVSGISWRGDST GYADSVKGRFTISRDNAKNTVDLQMNSLKPEDTAIYYCAAAAGSAWYGTLYEYDYWG QGTQVTVSSALE (SEQ ID NO: 187), or an amino acid sequence having at least 80% identity thereof. Another non-limiting example of a nanobody which targets EGFR is nanobody Nb 9G8 (anti-EGFR). In some embodiments, the nanobody Nb 9G8 (anti-EGFR) can comprise the amino acid sequence EVQLVESGGGLVQAGGSLRLSCAASGRTFSSYAMGWFRQAPGKEREFVVAINWSSGST YYADDSVKGRFTISRDNAKNTMYLQMNSLKPEDTAVYYCAAGYQINSGNYNFKDYEYD YWGQGTQVTVSSALE (SEQ ID NO: 188), or an amino acid sequence having at least 80% identity thereof.

In some embodiments, the targeting molecule can be a ligand (e.g., a natural receptor ligand), or a fragment or a derivative thereof, such as, but not limited to, EGFm123 (modified EGF), human stem cell factor (hSCF), and human insulin-like growth factor 1 (hIGF1). In some embodiments, EGFm123 of the present disclosure can comprise the amino acid sequence NSYSECPPSYDGYCLHDGVCRYIEALDSYACNCVVGYAGERCQYRDLRWWGRR (SEQ ID NO: 186), or an amino acid sequence having at least 80% identity thereof. In some embodiments, human stem cell factor (hSCF) of the present disclosure can comprise the amino acid sequence EGICRNRVTNNVKDVTKLVANLPKDYMITLKYVPGMDVLPSHCWISEMVVQLSDSLTD LLDKFSNISEGLSNYSIIDKLVNIVDDLVECVKENSSKDLKKSFKSPEPRLFTPEEFFRIFNR SIDAFKDFVVASETSDCVVSSTLSPEKDSRVSVTKPFMLPPVAA (SEQ ID NO: 189), or an amino acid sequence having at least 80% identity thereof. In some embodiments, human insulin-like growth factor 1 (hIGF1) of the present disclosure can comprise the amino acid sequence GPETLCGAELVDALQFVCGDRGFYFNKPTGYGSSSRRAPQTGIVDECCFRSCDLRRLEM YCAPLKPAKSA (SEQ ID NO: 190), or an amino acid sequence having at least 80% identity thereof.

In some embodiments, the targeting molecule disclosed herein can comprise a T cell receptor (TCR) or fragment or derivative thereof. A TCR can recognize a peptide presented in the context of a major histocompatibility complex (MHC) molecule. The peptide MHC (pMHC) complex can be recognized by the TCR, with the peptide (antigenic determinant) and the TCR idiotype providing the specificity of the interaction. Accordingly, an antigen described herein can encompass a peptide presented in the context of MHC molecules. The peptide which may be displayed on the MHC molecule can also comprise an epitope described herein. In some embodiments, the epitope can encompass not only those presented naturally by antigen-presenting cells (APCs) but may be any desired peptide so long as it is recognized by an immune cell, e.g., when presented appropriately to the cells of an immune system. For example, a peptide having an artificially prepared amino acid sequence may also be used as the epitope. In some embodiments, in addition to a fusogen, e.g., a rhabdovirus glycoprotein (G), such as but not limited to a VSV-G or a functional fragment or derivative thereof described herein, a viral particle described herein may further display a TCR-binding molecule. In some embodiments, the TCR-binding molecule and the fusogen can be comprised within a recombinant fusion protein described herein. In some embodiments, the TCR-binding molecule can comprise a TCR-specific antibody, or portion thereof.

In various embodiments, a TCR-binding molecule described herein can comprise a peptide presented in the context of a MHC molecule, e.g., is an antigenic determinant associated with(in) a peptide binding groove of an MHC. In some embodiments, a viral particle described herein, e.g., a viral particle comprising a recombinant fusogenic protein described herein, can be capable of binding to an antigen-specific T cell receptor (TCR), the recombinant viral particle comprising a lipid envelope comprising (i) a peptide (p) presented in the context of a major histocompatibility complex (MHC) molecule, i.e., a pMHC complex, and (ii) a fusogen. In some embodiments, the antigen-specific TCR specifically binds the pMHC complex.

MHC molecules are generally classified into two categories: class I and class II MHCs. An MHC class I molecule is an integral membrane protein comprising a glycoprotein heavy chain, also referred to herein as the α chain, which has three extracellular domains (i.e., α1, α2 and α3) and two intracellular domains (i.e., a transmembrane domain (TM) and a cytoplasmic domain (CYT)). The heavy chain is noncovalently associated with a soluble subunit called β2-microglobulin (β2m or B2M). An MHC class II protein is a heterodimeric integral membrane protein comprising one a chain and one p chain in noncovalent association. The α chain has two extracellular domains (α1 and α2), and two intracellular domains (a TM domain and a CYT domain). The β chain contains two extracellular domains (β1 and β2), and two intracellular domains (a TM domain and CYT domain).

The domain organization of class I and class II MHCs forms the antigenic determinant binding site, or peptide binding groove. A peptide binding groove refers to a portion of an MHC protein that forms a cavity in which a peptide, e.g., antigenic determinant, can bind. The conformation of a peptide binding groove is capable of being altered upon binding of a peptide to enable proper alignment of amino acid residues important for TCR binding to the peptide-MHC (pMHC) complex.

The MHCs described herein include fragments of MHC chains that are sufficient to form a peptide binding groove. For example, a peptide binding groove of a class I protein can comprise portions of the α1 and α2 domains of the heavy chain capable of forming two β-pleated sheets and two α helices. Inclusion of a portion of the β2-microglobulin chain stabilizes the complex. While for most versions of MHC class II molecules, interaction of the α and β chains can occur in the absence of a peptide, the two-chain complex of MHC class I is unstable until the binding groove is filled with a peptide. A peptide binding groove of a class II protein can comprise portions of the α1 and β1 domains capable of forming two β-pleated sheets and two α helices. A first portion of the α1 domain forms a first β-pleated sheet and a second portion of the α1 domain forms a first α helix. A first portion of the β1 domain forms a second β-pleated sheet and a second portion of the β1 domain forms a second a helix. The X-ray crystallographic structure of class II protein with a peptide engaged in the binding groove of the protein shows that one or both ends of the engaged peptide can project beyond the MHC protein. Thus, the ends of the α1 and β1α helices of class II form an open cavity such that the ends of the peptide bound to the binding groove are not buried in the cavity. Moreover, the X-ray crystallographic structure of class II proteins shows that the N-terminal end of the MHC β chain apparently projects from the side of the MHC protein in an unstructured manner since the first 4 amino acid residues of the β chain could not be assigned by X-ray crystallography.

Many human and other mammalian MHC molecules are well known in the art and any MHC class I or class II molecules may be part of a TCR-binding molecule as described herein.

MHC molecules useful in the viral particles described herein include naturally occurring full-length MHC molecules as well as individual chains of MHC molecules (e.g., MHC class I α (heavy) chain, β2-microglobulin, MHC class II α chain, and MHC class II β chain), individual subunits of such chains of MHCs (e.g., α1, α2 and/or α3 subunits of MHC class I α chain, α1 and/or α2 subunits of MHC class II α chain, β1 and/or β2 subunits of MHC class II β chain) as well as fragments, mutants and various derivatives thereof, wherein such fragments, mutants and derivatives retain the ability to display an antigenic determinant for recognition by an antigen-specific TCR. In one specific embodiment, the MHC comprises a transmembrane domain embedded in the lipid envelope of the viral particle.

Naturally-occurring MHC molecules are encoded by a cluster of genes on human chromosome 6 or mouse chromosome 17. Said MHCs, referred to as H-2 in mice and HLA (Human Leucocyte Antigen) in humans, are classified as either class I molecules or class II molecules. MHC class I molecules specifically bind CD8 molecules expressed on cytotoxic T lymphocytes (CD8+ T cells), whereas MHC class II molecules specifically bind CD4 molecules expressed on helper T lymphocytes (CD4+ T cells). MHCs include, but are not limited to, HLA specificities such as A (e.g., A1-A74), B (e.g., B 1-B77), C (e.g., C1-C11), D (e.g., D1-D26), E, G, DR (e.g., DR1-DR8), DQ (e.g., DQ1-DQ9) and DP (e.g., DP1-DP6). More preferably, HLA specificities include A1, A2, A3, A11, A23, A24, A28, A30, A33, B7, B8, B35, B44, B53, B60, B62, DR1, DR2, DR3, DR4, DR7, DR8, and DR-11.

The MHCs described herein may be from any mammalian or avian species, for example, primates (e.g., humans), rodents, rabbits, equines, bovines, canines, felines, pigs, etc.

Naturally occurring MHC class I molecules bind peptides derived from proteolytically degraded proteins, especially endogenously synthesized proteins, by a cell. Small peptides obtained accordingly are transported into the endoplasmic reticulum where they associate with nascent MHC class I molecules before being routed through the Golgi apparatus and displayed on the cell surface for recognition by cytotoxic T lymphocytes.

Naturally occurring MHC class I molecules consist of an α (heavy) chain associated with β2-microglobulin. The heavy chain consists of subunits α1-α3. The β2-microglobulin protein and α3 subunit of the heavy chain are associated. In certain embodiments, β2-microglobulin and α3 subunit are covalently bound. In certain embodiments, β2-microglobulin and α3 subunit are non-covalently bound. The α1 and α2 subunits of the heavy chain fold to form a groove for a peptide, e.g., antigenic determinant, to be displayed and recognized by TCR.

Class I molecules bind peptides of about 8-9 amino acids in length. All humans have between three and six different class I molecules, which can each bind many different types of peptides.

In some embodiments, the MHC comprises (i) a class I MHC polypeptide or a fragment, mutant or derivative thereof, and, optionally, (ii) a β2 microglobulin polypeptide or a fragment, mutant or derivative thereof. In one specific embodiment, the class I MHC polypeptide is linked to the β2 microglobulin polypeptide by a peptide linker.

In some embodiments, the class I MHC polypeptide is a human class I MHC polypeptide selected from the group consisting of HLA-A, HLA-B, HLA-C, HLA-E, HLA-F, and HLA-G. In another specific embodiment, the class I MHC polypeptide is a murine class I MHC polypeptide selected from the group consisting of H-2K, H-2D, H-2L, H2-IA, H2-IB, H2-IJ, H2-IE, and H2-IC.

In some embodiments, the viral particle comprises one or more MHC class I α heavy chains. In some embodiments, the MHC class I α heavy chain is fully human. In some embodiments, the MHC class I α heavy chain is humanized. Humanized MHC class I α heavy chains are described, e.g., in U.S. Pat. Pub. Nos. 2013/0111617, 2013/0185819 and 2014/0245467, both of which are incorporated herein by reference in their entireties. In some embodiments, the MHC class I α heavy chain comprises a human extracellular domain (human α1, α2, and/or α3 domains) and a cytoplasmic domain of another species. In some embodiments, the class I α heavy chain polypeptide is HLA-A, HLA-B, HLA-C, HLA-E, HLA-F, HLA-G, HLA-K, or HLA-L. In some embodiments, the HLA-A sequence can be an HLA-A*0201 sequence. In various aspects, the peptide-MHC can include all the domains of an MHC class I heavy chain.

In some embodiments, the viral particle comprises a β2-microglobulin. In some embodiments, the β2-microglobulin is fully human. In some embodiments, the β2-microglobulin is humanized. Humanized β2-microglobulin polypeptides are described, e.g., in U.S. Pat. Pub. Nos. 2013/0111617 and 2013/0185819, both of which are incorporated herein by reference in their entireties.

In some embodiments, the MHC class I molecule comprises a mutation in a 02-microglobulin (β2m or B2M) polypeptide and in the Heavy Chain sequence so as to affect a disulfide bond between the B2M and the Heavy Chain. In some cases, the Heavy Chain is an HLA and wherein the disulfide bond links one of the following pairs of residues: B2M residue 12, HLA residue 236; B2M residue 12, HLA residue 237; B2M residue 8, HLA residue 234; B2M residue 10, HLA residue 235; B2M residue 24, HLA residue 236; B2M residue 28, HLA residue 232; B2M residue 98, HLA residue 192; B2M residue 99, HLA residue 234; B2M residue 3, HLA residue 120; B2M residue 31, HLA residue 96; B2M residue 53, HLA residue 35; B2M residue 60, HLA residue 96; B2M residue 60, HLA residue 122; B2M residue 63, HLA residue 27; B2M residue Arg3, HLA residue Gly120; B2M residue His31, HLA residue Gln96; B2M residue Asp53, HLA residue Arg35; B2M residue Trp60, HLA residue Gln96; B2M residue Trp60, HLA residue Asp122; B2M residue Tyr63, HLA residue Tyr27; B2M residue Lys6, HLA residue Glu232; B2M residue Gln8, HLA residue Arg234; B2M residue TyrlO, HLA residue Pro235, B2M residue Serl 1, HLA residue Gln242; B2M residue Asn24, HLA residue Ala236; B2M residue Ser28, HLA residue Glu232; B2M residue Asp98, HLA residue His 192; and B2M residue Met99, HLA residue Arg234, first linker position Gly 2, Heavy Chain (HLA) position Tyr 84; Light Chain (B2M) position Arg 12, HLA Ala236; and/or B2M residue Argl2, HLA residue Gly237. See, e.g., Int. Pat. Appl. Pub. WO2015/195531.

In some embodiments, the antigenic determinant amino acid sequence can be that of a peptide which can be presented by an MHC class I molecule. In certain embodiments, the sequence can comprise from about 8 to about 15 contiguous amino acids. In certain embodiments, a peptide sequence can be that of a protein fragment, wherein the protein is a derived from, e.g., a portion of, an infectious agent or a cellular protein, such as, for example, a protein expressed by a cancer cell, and wherein the peptide can be bound to the MHC class I heavy chain.

In some embodiments, at least one chain of the MHC and the peptide are comprised within a fusion protein described herein. In one specific embodiment, the MHC and the peptide are separated by a linker sequence. For example, the single chain molecule can comprise, from amino to carboxy terminal, an antigenic determinant, a β2-microglobulin sequence, and a class I α (heavy) chain sequence. Alternatively, the single chain molecule can comprise, from amino to carboxy terminal, an antigenic determinant, a class I α (heavy) chain sequence, and a β2-microglobulin sequence. The single-chain molecule can further comprise a signal peptide sequence at the amino terminal. In certain embodiments, there can be a linker sequence between the peptide sequence and the β2-microglobulin sequence. In certain embodiments, there can be a linker sequence between the β2-microglobulin sequence and the class I α (heavy) chain sequence. A single-chain molecule can further comprise a signal peptide sequence at the amino terminal, as well as first linker sequence extending between the peptide sequence and the β2-microglobulin sequence, and/or a second linker sequence extending between the β2-microglobulin sequence and the class I heavy chain sequence. In certain embodiments, the β2-microglobulin and the class I α (heavy) chain sequences can be human, murine, or porcine.

In some embodiments, a single-chain molecule can comprise a first flexible linker between the peptide ligand segment and the β2-microglobulin segment. For example, linkers can extend from and connect the carboxy terminal of the peptide ligand segment to the amino terminal of the β2-microglobulin segment. Preferably, the linkers are structured to allow the linked peptide ligand to fold into the binding groove resulting in a functional MHC-antigen peptide. In some embodiments, this linker can comprise at least about 10 amino acids, up to about 15 amino acids. In some embodiments, a single-chain molecule can comprise a second flexible linker inserted between the β2-microglobulin and heavy chain segments. For example, linkers can extend from and connect the carboxy terminal of the β2-microglobulin segment to the amino terminal of the heavy chain segment. In certain embodiments, the β2-microglobulin and the heavy chain can fold into the binding groove resulting in a molecule which can function in promoting T cell expansion.

Suitable linkers used in the MHCs can be of any of a number of suitable lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and can be 1, 2, 3, 4, 5, 6, or 7 amino acids. Non-limiting examples of linkers include, e.g., glycine polymers (G)n, glycine-serine polymers (including, for example, (GS)n, (GSGGS (SEQ ID NO: 44)) and (GGGS (SEQ ID NO: 45)), where n is an integer of at least one, glycine-alanine polymers, alanine-serine polymers, and other flexible linkers. Glycine and glycine-serine polymers can be used; both Gly and Ser are relatively unstructured, and therefore can serve as a neutral tether between components. Glycine polymers can be used; glycine accesses significantly more phi-psi space than even alanine and is much less restricted than residues with longer side chains. Exemplary linkers can comprise amino acid sequences including, but not limited to, GGSG (SEQ ID NO: 46), GGSGG (SEQ ID NO: 47), GSGSG (SEQ ID NO: 48), GSGGG (SEQ ID NO: 49), GGGSG (SEQ ID NO: 50), GSSSG (SEQ ID NO: 51), GCGASGGGGSGGGGS (SEQ ID NO: 52), GGGGSGGGGS (SEQ ID NO: 53), GGGASGGGGSGGGGS (SEQ ID NO: 54), GGGGSGGGGSGGGGS (SEQ ID NO: 55), GGGASGGGGS (SEQ ID NO: 56), or GGGGSGGGGSGGGGSGGGGS (SEQ ID NO: 57), and the like. In some embodiments, a linker polypeptide includes a cysteine residue that can form a disulfide bond with a cysteine residue present in a second polypeptide.

In certain embodiments, the single-chain molecule can comprise a peptide covalently attached to an MHC class I α (heavy) chain via a disulfide bridge (i.e., a disulfide bond between two cystines). In certain embodiments, the disulfide bond comprises a first cysteine, comprised by a linker extending from the carboxy terminal of an antigen peptide, and a second cysteine comprised by an MHC class I heavy chain (e.g., an MHC class I α (heavy) chain which has a non-covalent binding site for the antigen peptide). In certain embodiments, the second cysteine can be a mutation (addition or substitution) in the MHC class I α (heavy) chain. In certain embodiments, the single-chain molecule can comprise one contiguous polypeptide chain as well as a disulfide bridge. In certain embodiments, the single-chain molecule can comprise two contiguous polypeptide chains which are attached via the disulfide bridge as the only covalent linkage. In some embodiments, the linking sequences can comprise at least one amino acid in addition to the cysteine, including one or more glycines, one or more, alanines, and/or one or more serines.

In certain embodiments, the disulfide bridge can link an antigen peptide in the class I groove of the pMHC complex if the pMHC complex comprises a first cysteine in a Gly-Ser linker extending between the C-terminus of the peptide and the β2-microglobulin, and a second cysteine in a proximal heavy chain position.

In some embodiments, the β2-microglobulin sequence can comprise a full-length β2-microglobulin sequence. In certain embodiments, the β2-microglobulin sequence lacks the leader peptide sequence. As such, in some configurations, the β2-microglobulin sequence can comprise about 99 amino acids and can be a mouse β2-microglobulin sequence (e.g., Genebank X01838). In some other configurations, the β2-microglobulin sequence can comprise about 99 amino acids and can be a human β2-microglobulin sequence (e.g., Genebank AF072097.1).

In some embodiments, the pMHC complex sequence can be that as disclosed in U.S. Pat. Nos. 4,478,82; 6,011,146; 8,518,697; 8,895,020; 8,992,937; WO 96/04314; Mottez et al. J. Exp. Med. 181: 493-502, 1995; Madden et al. Cell 70: 1035-1048, 1992; Matsumura et al., Science 257: 927-934, 1992; Mage et al., Proc. Natl. Acad. Sci. USA 89: 10658-10662, 1992; Toshitani et al, Proc. Nat'l Acad. Sci. 93: 236-240, 1996; Chung et al, J. Immunol. 163:3699-3708, 1999; Uger and Barber, J. Immunol. 160: 1598-1605, 1998; Uger et al., J. Immunol. 162, pp. 6024-6028, 1999; White et al., J. Immunol. 162: 2671-2676, 1999; Yu et al., J. Immunol. 168:3145-3149, 2002; Truscott et al., J. Immunol. 178: 6280-6289, 2007, each of which is incorporated herein by reference in its entirety for all purposes.

In some embodiments, the MHC comprises a class II MHC polypeptide or a fragment, mutant or derivative thereof. In one specific embodiment, the MHC comprises a and 3 polypeptides of a class II MHC complex or a fragment, mutant or derivative thereof. In one specific embodiment, the α and β polypeptides are linked by a peptide linker. In one specific embodiment, the MHC comprises α and β polypeptides of a human class II MHC complex selected from the group consisting of HLA-DP, HLA-DR, HLA-DQ, HLA-DM and HLA-DO. In another specific embodiment, the MHC comprises α and β polypeptides of a murine H-2A or H-2E class II MHC complex.

Naturally occurring MHC class II molecules consist of two polypeptide chains, a and 1. The chains may come from the DP, DQ, or DR gene groups. There are about 40 known different human MHC class II molecules. All have the same basic structure but vary subtly in their molecular structure. MHC class II molecules bind peptides of 13-18 amino acids in length.

In some embodiments, the viral particle comprises one or more MHC class II α chains. In some embodiments, the MHC class II α chain is fully human. In some embodiments, the MHC class II α chain is humanized. Humanized MHC class II α chains are described, e.g., in U.S. Pat. Nos. 8,847,005 and 9,043,996 and U.S. Pat. Pub. No. 2014/0245467, each of which is incorporated herein by reference in its entirety for all purposes. In some embodiments, the humanized MHC class II α chain polypeptide comprises a human extracellular domain and a cytoplasmic domain of another species. In some embodiments, the class II α chain is HLA-DMA, HLA-DOA, HLA-DPA, HLA-DQA or HLA-DRA. In some embodiments, the class II α chain polypeptide is humanized HLA-DMA, HLA-DOA, HLA-DPA, HILA-DQA and/or HLA-DRA.

In some embodiments, the viral particle comprises one or more MHC class II chains. In some embodiments, the MHC class II β chain is fully human. In some embodiments, the MHC class II β chain polypeptide is humanized. Humanized MHC class II β chain polypeptides are described, e.g., in U.S. Pat. Nos. 8,847,005 and 9,043,996 and U.S. Pat. Pub. No. 2014/0245467, each of which is incorporated herein by reference in its entirety for all purposes. In some embodiments, the humanized MHC class II β chain comprises a human extracellular domain and a cytoplasmic domain of another species. In some embodiments, the class II β chain is HLA-DMB, HLA-DOB, HILA-DPB, HLA-DQB or HLA-DRB. In some embodiments, the class II β chain is humanized HLA-DMB, HLA-DOB, HLA-DPB, HLA-DQB and/or HLA-DRB.

In some embodiments, a peptide which may be useful for targeting a rhabdovirus glycoprotein (G), or a functional fragment or derivative thereof, described herein may be synthetically produced or produced by hydrolysis. Synthetically produced peptides can include randomly generated peptides, specifically designed peptides, and peptides where at least some of the amino acid positions are conserved among several peptides and the remaining positions are random. Alternatively, a peptide of the present disclosure may be produced by expression in a heterologous host cell.

In some embodiments, a peptide of the disclosure may be from about 5 to about 40 amino acid residues, more preferably from about 6 to about 30 amino acid residues, and even more preferably from about 8 to about 20 amino acid residues, and even more preferably between about 9 and 11 amino acid residues. In some embodiments, a peptide can include any size peptide, e.g., between 5 and 200 amino acids in length, in whole integer increments (i.e., 5, 6, 7, 8, 9 . . . 200).

The peptides of the disclosure may comprise one or more reverse peptide bonds, one or more non-peptide bonds, one or more chemical modifications, one or more D-isomers of amino acids, or any combination thereof.

Peptides described herein may comprise one or more (e.g., 1, 2, 3, or 4) amino acid substitutions and/or insertions and/or deletions. Amino acid substitution means that an amino acid residue is substituted for a replacement amino acid residue at the same position. Inserted amino acid residues may be inserted at any position and may be inserted such that some or all of the inserted amino acid residues are immediately adjacent one another or may be inserted such that none of the inserted amino acid residues is immediately adjacent another inserted amino acid residue.

Inserted amino acids and substituted amino acids may be naturally occurring amino acids or may be non-naturally occurring amino acids and, for example, may contain a non-natural side chain, and/or be linked together via non-native peptide bonds. If more than one amino acid residue is substituted and/or inserted, the replacement/inserted amino acid residues may be the same as each other or different from one another. Each replacement amino acid may have a different side chain to the amino acid being replaced.

Combinations of several substitutions/additions/deletions at more than one position can be developed and tested to determine if the combination results in an additive or synergistic effects on the peptide.

Multiple peptides described herein may be operably linked together. For example, such a multi-epitope peptide or polypeptide may comprise include 2 to 37, 2 to 30, 5 to 25, 5 to 20, or 10 to 15 single-epitope peptides. The single-epitope peptides may be linked via a linker described herein, or other such linker known to person of ordinary skill in the art to which this disclosure belongs.

A non-limiting example of a peptide which can be used in the practice of the present disclosure is peptide 27-24M which targets the tumor antigen HER2. In some embodiments, peptide 27-24M comprises the amino acid sequence NKFNKGMRGYWGALGGGNGKRGIMGYD (SEQ ID NO: 191), or an amino acid sequence having at least 80% identity thereof.

### Recombinant Polynucleotides

In one aspect, the present disclosure provides recombinant polynucleotide molecules encoding one or more of the above-described polypeptides. In some embodiments, a recombinant polynucleotide molecule can encode a fusogenic protein described herein. In some embodiments, the polynucleotide can comprise a sequence encoding a signal peptide sequence, and the signal peptide sequence can be positioned at the extreme N-terminus of the encoded recombinant fusogenic protein. In some embodiments, the polynucleotide is DNA. In some embodiments, the polynucleotide is RNA.

In one embodiment, provided herein is a recombinant polynucleotide and the recombinant polynucleotide is an RNA molecule comprising a nucleotide sequence that is a template for a positive sense transcript encoding a recombinant fusogenic protein described herein. In some embodiments, the positive sense transcript can comprise a sequence encoding a signal peptide sequence, and the signal peptide sequence can be positioned at the extreme N-terminus of the encoded recombinant fusogenic protein.

In one embodiment, provided herein is a recombinant polynucleotide and the recombinant polynucleotide is an RNA molecule comprising a nucleotide sequence that is a template for a positive sense transcript encoding a vesicular stomatitis virus (VSV) nucleoprotein (N) polypeptide or a functional fragment or derivative thereof, a nucleotide sequence that is a template for a positive sense transcript encoding a VSV phosphoprotein (P) polypeptide or a functional fragment or derivative thereof, a nucleotide sequence that is a template for a positive sense transcript encoding a VSV matrix (M) polypeptide or a functional fragment or derivative thereof, a nucleotide sequence that is a template for a positive sense transcript encoding the fusogenic protein described herein, and a nucleotide sequence that is a template for a positive sense transcript encoding a VSV large protein (L) polypeptide or a functional fragment or derivative thereof.

In some embodiments, the VSV nucleoprotein (N) polypeptide comprises the amino acid sequence of SEQ ID NO: 180, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 180. In certain embodiments, the nucleotide sequence that encodes the VSV nucleoprotein (N) polypeptide comprises the nucleotide sequence that encodes the amino acid sequence of SEQ ID NO: 180, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 180. In certain embodiments, the nucleotide sequence that encodes the VSV nucleoprotein (N) polypeptide comprises the nucleotide sequence of SEQ ID NO: 181, or a nucleotide sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 181. In certain embodiments, the VSV nucleoprotein (N) polypeptide comprises the amino acid sequence of SEQ ID NO: 180. In certain embodiments, the nucleotide sequence that encodes the VSV nucleoprotein (N) polypeptide comprises the nucleotide sequence of SEQ ID NO: 181.

In some embodiments, the VSV nucleoprotein (N) polypeptide comprises the amino acid sequence of SEQ ID NO: 180 or an amino acid sequence that has at least 80% sequence identity to SEQ ID NO: 180.

In some embodiments, the VSV phosphoprotein (P) polypeptide comprises the amino acid sequence of SEQ ID NO: 182, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 182. In certain embodiments, the nucleotide sequence that encodes the VSV phosphoprotein (P) polypeptide comprises the nucleotide sequence that encodes the amino acid sequence of SEQ ID NO: 182, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 182. In certain embodiments, the nucleotide sequence that encodes the VSV phosphoprotein (P) polypeptide comprises the nucleotide sequence of SEQ ID NO: 183, or a nucleotide sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 183. In certain embodiments, the VSV phosphoprotein (P) polypeptide comprises the amino acid sequence of SEQ ID NO: 182. In certain embodiments, the nucleotide sequence that encodes the VSV phosphoprotein (P) polypeptide comprises the nucleotide sequence of SEQ ID NO: 183.

In some embodiments, the VSV phosphoprotein (P) polypeptide comprises the amino acid sequence of SEQ ID NO: 182 or an amino acid sequence that has at least 80% sequence identity to SEQ ID NO: 182.

In some embodiments, the VSV M polypeptide is a mutant VSV M polypeptide. In some embodiments, the mutant VSV M polypeptide comprises a mutation at methionine (M) 51. In some embodiments, the mutation at methionine (M) 51 is a substitution from methionine (M) to arginine (R). In some embodiments, the mutant VSV M polypeptide may comprise a deletion at methionine (M) 51 (ΔM51).

In some embodiments, the wild-type VSV matrix (M) polypeptide comprises the amino acid sequence of SEQ ID NO: 138, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 138. In certain embodiments, the nucleotide sequence that encodes the wild-type VSV matrix (M) polypeptide comprises the nucleotide sequence that encodes the amino acid sequence of SEQ ID NO: 138, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 138. In certain embodiments, the nucleotide sequence that encodes the wild-type VSV matrix (M) polypeptide comprises the nucleotide sequence of SEQ ID NO: 139, or a nucleotide sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 139. In certain embodiments, the wild-type VSV matrix (M) polypeptide comprises the amino acid sequence of SEQ ID NO: 138. In certain embodiments, the nucleotide sequence that encodes the wild-type VSV matrix (M) polypeptide comprises the nucleotide sequence of SEQ ID NO: 139.

In some embodiments, the VSV M polypeptide comprises the amino acid sequence of SEQ ID NO: 138 or an amino acid sequence that has at least 80% sequence identity to SEQ ID NO: 138.

In some embodiments, the mutant VSV matrix (M) polypeptide M51R comprises the amino acid sequence of SEQ ID NO: 140, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 140. In certain embodiments, the nucleotide sequence that encodes the mutant VSV matrix (M) polypeptide M51R comprises the nucleotide sequence that encodes the amino acid sequence of SEQ ID NO: 140, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 140. In certain embodiments, the nucleotide sequence that encodes the mutant VSV matrix (M) polypeptide M51R comprises the nucleotide sequence of SEQ ID NO: 141, or a nucleotide sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 141. In certain embodiments, the mutant VSV matrix (M) polypeptide M51R comprises the amino acid sequence of SEQ ID NO: 140. In certain embodiments, the nucleotide sequence that encodes the mutant VSV matrix (M) polypeptide M51R comprises the nucleotide sequence of SEQ ID NO: 141.

In some embodiments, the VSV M polypeptide comprises the amino acid sequence of SEQ ID NO: 140 or an amino acid sequence that has at least 80% sequence identity to SEQ ID NO: 104.

In some embodiments, the mutant VSV matrix (M) polypeptide ΔM51 comprises the amino acid sequence of SEQ ID NO: 142, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 142. In certain embodiments, the nucleotide sequence that encodes the mutant VSV matrix (M) polypeptide ΔM51 comprises the nucleotide sequence that encodes the amino acid sequence of SEQ ID NO: 142, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 142. In certain embodiments, the nucleotide sequence that encodes the mutant VSV matrix (M) polypeptide ΔM51 comprises the nucleotide sequence of SEQ ID NO: 143, or a nucleotide sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 143. In certain embodiments, the mutant VSV matrix (M) polypeptide ΔM51 comprises the amino acid sequence of SEQ ID NO: 142. In certain embodiments, the nucleotide sequence that encodes the mutant VSV matrix (M) polypeptide ΔM51 comprises the nucleotide sequence of SEQ ID NO: 143.

In some embodiments, the VSV M polypeptide comprises the amino acid sequence of SEQ ID NO: 142 or an amino acid sequence that has at least 80% sequence identity to SEQ ID NO: 142.

In some embodiments, the VSV large protein (L) polypeptide comprises the amino acid sequence of SEQ ID NO: 184, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 184. In certain embodiments, the nucleotide sequence that encodes the VSV large protein (L) polypeptide comprises the nucleotide sequence that encodes the amino acid sequence of SEQ ID NO: 184, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 184. In certain embodiments, the nucleotide sequence that encodes the VSV large protein (L) polypeptide comprises the nucleotide sequence of SEQ ID NO: 185, or a nucleotide sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 185. In certain embodiments, the VSV large protein (L) polypeptide comprises the amino acid sequence of SEQ ID NO: 184. In certain embodiments, the nucleotide sequence that encodes the VSV large protein (L) polypeptide comprises the nucleotide sequence of SEQ ID NO: 185.

In some embodiments, the VSV large protein (L) polypeptide comprises the amino acid sequence of SEQ ID NO: 184 or an amino acid sequence that has at least 80% sequence identity to SEQ ID NO: 184.

In certain embodiments, the polynucleotide encoding the polypeptides disclosed herein may comprise one or more regulatory elements. The regulatory element may be capable of modulating expression of the polypeptides. Non-limiting examples of regulatory elements are, promoters, initiation sites, polyadenylation (polyA) tails, IRES elements, enhancers, response elements, and termination signals. In some embodiments, the promoter is an inducible promoter.

Nucleic acid inserted into the genome of a VSV can be flanked by viral intragenic regions containing the gene transcription start and stop codes required for transcription of the inserted nucleic acid sequences by the viral polymerase.

In some embodiments, a polynucleotide described herein is optimized for expression in human cells.

### Recombinant Pseudotyped Viruses and Cell-Derived Nanovesicles

In certain aspects, the present disclosure provides a recombinant pseudotyped virus or cell-derived nanovesicle comprising one or more recombinant fusogenic proteins described herein or a recombinant polynucleotide described herein.

In some embodiments, the recombinant fusogenic protein forms a chimeric trimer with one or two different fusogenic proteins on the surface of the recombinant pseudotyped virus or cell-derived nanovesicle. In some embodiments, the chimeric trimer comprises (i) at least one fusogenic protein described herein, and (ii) a wild-type rhabdoviral glycoprotein and/or a recombinant fusogenic protein comprising a rhabdoviral glycoprotein, or a functional fragment or derivative thereof, and a targeting molecule. These chimeric trimers may be distributed across the surface of said recombinant pseudotyped virus or cell-derived nanovesicle such that all trimers include at least one targeting molecule, or that not every trimer does so. In the latter case, some trimers may display one targeting molecule, others two targeting molecules, others none. In some embodiments, the chimeric trimer can comprise (i) at least one fusogenic protein described herein, and (ii) a fusogenic protein comprising a rhabdoviral glycoprotein, or a functional fragment or derivative thereof, without a targeting molecule. In some embodiments, a chimeric trimer described herein may comprise two or more different recombinant fusogenic proteins described herein.

In some embodiments, a fusogenic protein described herein may comprise a fusogen that comprises the sequence SEQ ID NO: 8, with amino acid substitutions and/or deletions at one or more positions selected from K47, R354, H8, and Y209.

In some embodiments a recombinant pseudotyped virus of cell-derived nanonvesicle described herein may comprise a chimeric trimer comprising (i) one or two monomers of a first fusogenic protein, and the first fusogenic protein comprises a rhabdovirus glycoprotein, or a functional fragment or derivative thereof, and a targeting molecule, or the functional fragment or derivative thereof, and (ii) one or two monomers of a second fusogenic protein, and the second fusogenic protein comprises a rhabdovirus glycoprotein, or a functional fragment or derivative thereof, without a targeting molecule.

In some embodiments, the targeting molecule can be attached to the rhabdovirus glycoprotein via a linker. In some embodiments, the linker is not sensitive to a proteolytic cleavage by an endogenous protease or by an exogenously added protease. In some embodiments, the linker is sensitive to a proteolytic cleavage by an endogenous protease or by an exogenously added protease. In some embodiments, the linker may be any of various linkers described herein.

In some embodiments, the first fusogenic protein and/or the second fusogenic protein can comprises a rhabdovirus glycoprotein that comprises the sequence SEQ ID NO: 8, with amino acid substitutions and/or deletions at one or more positions selected from K47, R354, H8, and Y209.

Cell-derived nanovesicles can be natural or engineered nanosized vesicles that can carry various biologicals materials such as proteins, polynucleotides, or lipids. One example of a cell-derived nanovesicle is gesicle. A gesicle is a cell-derived nanovesicle released from the cell's plasma membrane and is typically produced through overexpression of vesicular stomatitis virus G (VSV-G) glycoprotein (Campbell, L. A. et. al., Mol. Ther., 2019, 27, p. 151-163; Mangeot, P. E. et. al., Mol. Ther., 2011, 19, p. 1656-1666, both of which are incorporated herein by reference in their entireties). Measuring 100 nm in diameter on average, gesicles are heterogeneous in size, shape, and cargo, and can be used for the rapid and direct transfer of membrane, cytoplasmic, and nuclear proteins into recipient cells. Gesicles can be a viable approach to deliver genome editing agents (e.g., CRISPR/Cas9 ribonucleoproteins (RNPs)) to target cells. As opposed to gene delivery by viral vectors, protein delivery by gesicles allows a rapid transfer of function in cells without the involvement of transcription machinery or any viral integration process that could limit viral transduction in specific cell types.

Another example of a cell-derived nanovesicle is a Nanoblade. Nanoblades are engineered particles loaded with a genome editing agent (e.g., Cas9-gRNA ribonucleoproteins (RNPs)) (Mangeot, P. E. et. al., Nat. Commun., 2019, 45, p. 1-15; Gutierrez-Guerrero, A. et. al., Front. Genome Ed., 2021, 3, p. 1-21, both of which are incorporated herein by reference in their entireties). Nanoblades are formed when viral structural proteins, such as the MLV protein Gag, multimerize and spontaneously assemble into particles at the cell membrane. These particles can incorporate one or more guide RNAs through association with Cas9 and act as a delivery agent into target cells. To alter the cell tropism of Nanoblades, different viral envelop proteins can be expressed to pseudotype Nanoblades. For example, Nanoblades pseudotyped with a mix of VSV-G and the baboon endogenous retrovirus Rless glycoprotein (BaEVRless) have been shown to have high rates of transduction in recipient cells (Mangeot, P. E. et. al., Nat. Commun., 2019, 45, p. 1-15).

In one aspect, the present disclosure also provides a recombinant pseudotyped virus or cell-derived nanovesicle comprising a glycoprotein from a different virus, or a functional fragment or derivative thereof. In some embodiments, the recombinant pseudotyped virus is a rhabdovirus, such as a vesicular stomatitis virus (VSV). In some embodiments, the rhabdovirus may be replication-competent. In some embodiments, the rhabdovirus may be replication-deficient. In some embodiments, the rhabdovirus may be non-replicative.

In some embodiments, the recombinant pseudotyped virus is a retrovirus, such as a lentivirus (LV). In some embodiments, the LV may be replication-competent. In some embodiments, the LV may be replication-deficient. In some embodiments, the LV may be non-replicative. In some embodiments, a LV described herein may not contain gp120 surface envelope protein and/or gp41 transmembrane envelope protein. In some embodiments, a LV described herein can contain a mutant gp120 surface envelope protein and/or a mutant gp41 transmembrane envelope protein. In some embodiments, a LV described herein may not be capable of binding to a cell, for example, in the absence of a TCR described herein.

In some embodiments, the recombinant pseudotyped virus described herein can comprise, for example, without limitation, components from a virus selected from the group consisting of Human Immunodeficiency Virus (e.g., HIV-1 or HIV-2), Bovine Immunodeficiency Virus (BIV), Feline Immunodeficiency Virus (FIV), Simian Immunodeficiency Virus (SIV), Equine Infectious Anemia Virus (EIAV), Murine Stem Cell Virus (MSCV), Murine Leukemia Virus (MLV), Avian leukosis virus (ALV), Feline leukemia virus (FLV), Bovine leukemia virus (BLV), Human T-lymphotropic virus (HTLV), feline sarcoma virus, avian reticuloendotheliosis virus, caprine arthritis encephalitis virus (CAEV), and Visna-Maedi virus (VMV).

In some embodiments, a recombinant pseudotyped virus or cell-derived nanovesicle of the present disclosure can be derived from a virus of the family Retroviridae.

In some embodiments, a recombinant pseudotyped virus or cell-derived nanovesicle of the present disclosure may comprise a glycoprotein from a rhabdovirus virus such as, without limitation, a vesicular stomatitis virus glycoprotein (VSV-G), a Flanders virus glycoprotein (FLAV-G), a Chandipura virus glycoprotein (CHPV-G), a Perinet virus glycoprotein (PERV-G), a Piry virus glycoprotein (PIRYV-G), a Fukuoka virus glycoprotein (FUKV-G), a Joinjakaka virus glycoprotein (JOIV-G), a Kumasi virus glycoprotein (KRV-G), a Isfahan glycoprotein (ISFV-G), a Jurona glycoprotein (JURV-G), a Mediterranean Bat glycoprotein (MBV-G), a Malpais Spring glycoprotein (MSPV-G), a Radi glycoprotein (RADV-G), a Rhinolophus affinis-G, a Yug Bugdanavoc glycoprotein (YBV-G), a Yinshui Bat glycoprotein (YSBV-G), a Keuraliba glycoprotein (KEUV-G), a Kimberley glycoprotein (KIMV-G), a Kanyawara glycoprotein (KYAV-G), a La Joya glycoprotein (LJV-G), a Mosquiero glycoprotein (MQOV-G), a Parry Creek glycoprotein (PCV-G), a Bas Congo glycoprotein (BASV-G), a Bovine Ephemeral fever glycoprotein (BEFV-G), a Curionopolis glycoprotein (CURV-G), a Drosophila melanogaster sigmavirus glycoprotein (DMelSV-G), a Niakha glycoprotein (NIAV-G), a Puerto almandras glycoprotein (PTAMV-G), or a Tupaia rhabdovirus (TUPTV-G), or a functional fragment or derivative thereof. Non-limiting examples of amino acid sequences of rhabdovirus G described herein are set forth in SEQ ID NO: 8-15, 17, 60-108, 157.

In one embodiment, a recombinant pseudotyped virus or cell-derived nanovesicle of the present disclosure comprises a glycoprotein from Flanders virus (FLAV-G), or a functional fragment or derivative thereof. In some embodiments, the FLAV-G comprises the amino acid sequence of SEQ ID NO: 9 or 81, or a functional fragment or derivative thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity with SEQ ID NO: 9 or 81. In certain embodiments, the nucleotide sequence that encodes the FLAV-G comprises the nucleotide sequence that encodes the amino acid sequence of SEQ ID NO: 9 or 81, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 9 or 81. In certain embodiments, the FLAV-G comprises the amino acid sequence of SEQ ID NO: 9 or 81.

In some embodiments, a recombinant pseudotyped virus or cell-derived nanovesicle described herein comprises a rhabdovirus glycoprotein from FLAV-G. In some embodiments, the FLAV-G comprises the sequence SEQ ID NO: 9. In some embodiments, the FLAV-G consists of the sequence SEQ ID NO: 9.

In one embodiment, a recombinant pseudotyped virus or cell-derived nanovesicle of the present disclosure comprises a glycoprotein from a Chandipura virus (CHPV-G), or a functional fragment or derivative thereof. In some embodiments, the CHPV-G comprises the amino acid sequence of SEQ ID NO:10 or 82, or a functional fragment or derivative thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity with SEQ ID NO: 10 or 82. In certain embodiments, the nucleotide sequence that encodes the CHPV-G comprises the nucleotide sequence that encodes the amino acid sequence of SEQ ID NO: 10 or 82, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 10 or 82. In certain embodiments, the CHPV-G comprises the amino acid sequence of SEQ ID NO: 10 or 82.

In some embodiments, a recombinant pseudotyped virus or cell-derived nanovesicle described herein comprises a rhabdovirus glycoprotein from CHPV-G. In some embodiments, the CHPV-G comprises the sequence SEQ ID NO: 10. In some embodiments, the CHPV-G consists of the sequence SEQ ID NO: 10.

In one embodiment, a recombinant pseudotyped virus or cell-derived nanovesicle of the present disclosure comprises a glycoprotein from a Perinet virus (PERV-G), or a functional fragment or derivative thereof. In some embodiments, the PERV-G comprises the amino acid sequence of SEQ ID NO:11 or 87, or a functional fragment or derivative thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity with SEQ ID NO: 11 or 87. In certain embodiments, the nucleotide sequence that encodes the PERV-G comprises the nucleotide sequence that encodes the amino acid sequence of SEQ ID NO: 11 or 87, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 11 or 87. In certain embodiments, the PERV-G comprises the amino acid sequence of SEQ ID NO: 11 or 87.

In some embodiments, a recombinant pseudotyped virus or cell-derived nanovesicle described herein comprises a rhabdovirus glycoprotein from PERV-G. In some embodiments, the PERV-G comprises the sequence SEQ ID NO: 11. In some embodiments, the PERV-G consists of the sequence SEQ ID NO: 11.

In one embodiment, a recombinant pseudotyped virus or cell-derived nanovesicle of the present disclosure comprises a glycoprotein from a Piry virus (PIRYV-G), or a functional fragment or derivative thereof. In some embodiments, the PIRYV-G comprises the amino acid sequence of SEQ ID NO: 12 or 88, or a functional fragment or derivative thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity with SEQ ID NO: 12 or 88. In certain embodiments, the nucleotide sequence that encodes the PIRYV-G comprises the nucleotide sequence that encodes the amino acid sequence of SEQ ID NO: 12 or 88, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 12 or 88. In certain embodiments, the PIRYV-G comprises the amino acid sequence of SEQ ID NO: 12 or 88.

In some embodiments, a recombinant pseudotyped virus or cell-derived nanovesicle described herein comprises a rhabdovirus glycoprotein from PIRYV-G. In some embodiments, the PIRYV-G comprises the sequence SEQ ID NO: 12. In some embodiments, the PIRYV-G consists of the sequence SEQ ID NO: 12.

In one embodiment, a recombinant pseudotyped virus or cell-derived nanovesicle of the present disclosure comprises a glycoprotein from a Fukuoka virus (FUKV-G), or a functional fragment or derivative thereof. In some embodiments, the FUKV-G comprises the amino acid sequence of SEQ ID NO: 13 or 93, or a functional fragment or derivative thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity with SEQ ID NO: 13 or 93. In certain embodiments, the nucleotide sequence that encodes the FUKV-G comprises the nucleotide sequence that encodes the amino acid sequence of SEQ ID NO: 13 or 93, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 13 or 93. In certain embodiments, the FUKV-G comprises the amino acid sequence of SEQ ID NO: 13 or 93.

In some embodiments, a recombinant pseudotyped virus or cell-derived nanovesicle described herein comprises a rhabdovirus glycoprotein from FUKV-G. In some embodiments, the FUKV-G comprises the sequence SEQ ID NO: 13. In some embodiments, the FUKV-G consists of the sequence SEQ ID NO: 13.

In one embodiment, a recombinant pseudotyped virus or cell-derived nanovesicle of the present disclosure comprises a glycoprotein from a Joinjakaka virus (JOIV-G), or a functional fragment or derivative thereof. In some embodiments, the JOIV-G comprises the amino acid sequence of SEQ ID NO: 14 or 94, or a functional fragment or derivative thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity with SEQ ID NO: 14 or 94. In certain embodiments, the nucleotide sequence that encodes the JOIV-G comprises the nucleotide sequence that encodes the amino acid sequence of SEQ ID NO: 14 or 94, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 14 or 94. In certain embodiments, the JOIV-G comprises the amino acid sequence of SEQ ID NO: 14 or 94.

In some embodiments, a recombinant pseudotyped virus or cell-derived nanovesicle described herein comprises a rhabdovirus glycoprotein from JOIV-G. In some embodiments, the JOIV-G comprises the sequence SEQ ID NO: 14. In some embodiments, the JOIV-G consists of the sequence SEQ ID NO: 14.

In one embodiment, a recombinant pseudotyped virus or cell-derived nanovesicle of the present disclosure comprises a glycoprotein from a Kumasi virus (KRV-G), or a functional fragment or derivative thereof. In some embodiments, the KRV-G comprises the amino acid sequence of SEQ ID NO: 15 or 97, or a functional fragment or derivative thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity with SEQ ID NO: 15 or 97. In certain embodiments, the nucleotide sequence that encodes the KRV-G comprises the nucleotide sequence that encodes the amino acid sequence of SEQ ID NO: 15 or 97, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 15 or 97. In certain embodiments, the KRV-G comprises the amino acid sequence of SEQ ID NO: 15 or 97.

In some embodiments, a recombinant pseudotyped virus or cell-derived nanovesicle described herein comprises a rhabdovirus glycoprotein from KRV-G. In some embodiments, the KRV-G comprises the sequence SEQ ID NO: 15. In some embodiments, the KRV-G consists of the sequence SEQ ID NO: 15.

In some embodiments, a recombinant pseudotyped virus or cell-derived nanovesicle of the present disclosure comprises a glycoprotein from a Keuraliba virus (KEUV-G), or a functional fragment or derivative thereof. In some embodiments, the KEUV-G comprises the amino acid sequence of SEQ ID NO: 17 or 95, or a functional fragment or derivative thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity with SEQ ID NO: 17 or 95. In certain embodiments, the nucleotide sequence that encodes the KEUV-G comprises the nucleotide sequence that encodes the amino acid sequence of SEQ ID NO: 17 or 95, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 17 or 95. In certain embodiments, the KEUV-G comprises the amino acid sequence of SEQ ID NO: 17 or 95.

In some embodiments, a recombinant pseudotyped virus or cell-derived nanovesicle described herein comprises a rhabdovirus glycoprotein from KEUV-G. In some embodiments, the KEUV-G comprises the sequence SEQ ID NO: 17. In some embodiments, the KEUV-G consists of the sequence SEQ ID NO: 17.

In some embodiments, a recombinant pseudotyped virus or cell-derived nanovesicle of the present disclosure comprises a glycoprotein from an Isfahan virus (ISFV-G). In some embodiments, the ISFV-G comprises the amino acid sequence of SEQ ID NO: 60 or 83, or a functional fragment or derivative thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity with SEQ ID NO: 60 or 83. In certain embodiments, the nucleotide sequence that encodes the ISFV-G comprises the nucleotide sequence that encodes the amino acid sequence of SEQ ID NO: 60 or 83, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 60 or 83. In certain embodiments, the ISFV-G comprises the amino acid sequence of SEQ ID NO: 60 or 83.

In some embodiments, a recombinant pseudotyped virus or cell-derived nanovesicle described herein comprises a rhabdovirus glycoprotein from ISFV-G. In some embodiments, the ISFV-G comprises the sequence SEQ ID NO: 60. In some embodiments, the ISFV-G consists of the sequence SEQ ID NO: 60.

In some embodiments, a recombinant pseudotyped virus or cell-derived nanovesicle of the present disclosure comprises a glycoprotein from an Jurona virus (JURV-G). In some embodiments, the JURV-G comprises the amino acid sequence of SEQ ID NO: 61 or 84, or a functional fragment or derivative thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity with SEQ ID NO: 61 or 84. In certain embodiments, the nucleotide sequence that encodes the JURV-G comprises the nucleotide sequence that encodes the amino acid sequence of SEQ ID NO: 61 or 84, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 61 or 84. In certain embodiments, the JURV-G comprises the amino acid sequence of SEQ ID NO: 61 or 84.

In some embodiments, a recombinant pseudotyped virus or cell-derived nanovesicle described herein comprises a rhabdovirus glycoprotein from JURV-G. In some embodiments, the JURV-G comprises the sequence SEQ ID NO: 61. In some embodiments, the JURV-G consists of the sequence SEQ ID NO: 61.

In some embodiments, a recombinant pseudotyped virus or cell-derived nanovesicle of the present disclosure comprises a glycoprotein from a Mediterranean Bat virus (MBV-G). In some embodiments, the MBV-G comprises the amino acid sequence of SEQ ID NO: 62 or 85, or a functional fragment or derivative thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity with SEQ ID NO: 62 or 85. In certain embodiments, the nucleotide sequence that encodes the MBV-G comprises the nucleotide sequence that encodes the amino acid sequence of SEQ ID NO: 62 or 85, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 62 or 85. In certain embodiments, the MBV-G comprises the amino acid sequence of SEQ ID NO: 62 or 85.

In some embodiments, a recombinant pseudotyped virus or cell-derived nanovesicle described herein comprises a rhabdovirus glycoprotein from MBV-G. In some embodiments, the MBV-G comprises the sequence SEQ ID NO: 62. In some embodiments, the MBV-G consists of the sequence SEQ ID NO: 62.

In some embodiments, a recombinant pseudotyped virus or cell-derived nanovesicle of the present disclosure comprises a glycoprotein from a Malpais Spring virus (MSPV-G). In some embodiments, the MSPV-G comprises the amino acid sequence of SEQ ID NO: 63 or 86, or a functional fragment or derivative thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity with SEQ ID NO: 63 or 86. In certain embodiments, the nucleotide sequence that encodes the MSPV-G comprises the nucleotide sequence that encodes the amino acid sequence of SEQ ID NO: 63 or 86, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 63 or 86. In certain embodiments, the MSPV-G comprises the amino acid sequence of SEQ ID NO: 63 or 86.

In some embodiments, a recombinant pseudotyped virus or cell-derived nanovesicle described herein comprises a rhabdovirus glycoprotein from MSPV-G. In some embodiments, the MSPV-G comprises the sequence SEQ ID NO: 63. In some embodiments, the MSPV-G consists of the sequence SEQ ID NO: 63.

In some embodiments, a recombinant pseudotyped virus or cell-derived nanovesicle of the present disclosure comprises a glycoprotein from a Radi virus (RADV-G). In some embodiments, the RADV-G comprises the amino acid sequence of SEQ ID NO: 64 or 89, or a functional fragment or derivative thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity with SEQ ID NO: 64 or 89. In certain embodiments, the nucleotide sequence that encodes the RADV-G comprises the nucleotide sequence that encodes the amino acid sequence of SEQ ID NO: 64 or 89, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 64 or 89. In certain embodiments, the RADV-G comprises the amino acid sequence of SEQ ID NO: 64 or 89.

In some embodiments, a recombinant pseudotyped virus or cell-derived nanovesicle described herein comprises a rhabdovirus glycoprotein from RADV-G. In some embodiments, the RADV-G comprises the sequence SEQ ID NO: 64. In some embodiments, the RADV-G consists of the sequence SEQ ID NO: 64.

In some embodiments, a recombinant pseudotyped virus or cell-derived nanovesicle of the present disclosure comprises a glycoprotein from a Rhinolophus affinis virus (Rhinolophus affinis G). In some embodiments, the Rhinolophus affinis G comprises the amino acid sequence of SEQ ID NO: 65 or 90, or a functional fragment or derivative thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity with SEQ ID NO: 65 or 90. In certain embodiments, the nucleotide sequence that encodes the Rhinolophus affinis G comprises the nucleotide sequence that encodes the amino acid sequence of SEQ ID NO: 65 or 90, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 65 or 90. In certain embodiments, the Rhinolophus affinis G comprises the amino acid sequence of SEQ ID NO: 65 or 90.

In some embodiments, a recombinant pseudotyped virus or cell-derived nanovesicle described herein comprises a rhabdovirus glycoprotein from Rhinolophus affinis G. In some embodiments, the Rhinolophus affinis G comprises the sequence SEQ ID NO: 65. In some embodiments, the Rhinolophus affinis G consists of the sequence SEQ ID NO: 65.

In some embodiments, a recombinant pseudotyped virus or cell-derived nanovesicle of the present disclosure comprises a glycoprotein from a Yug Bugdanavoc virus (YBV-G). In some embodiments, the YBV-G comprises the amino acid sequence of SEQ ID NO: 66 or 91, or a functional fragment or derivative thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity with SEQ ID NO: 66 or 91. In certain embodiments, the nucleotide sequence that encodes the YBV-G comprises the nucleotide sequence that encodes the amino acid sequence of SEQ ID NO: 66 or 91, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 66 or 91. In certain embodiments, the YBV-G comprises the amino acid sequence of SEQ ID NO: 66 or 91.

In some embodiments, a recombinant pseudotyped virus or cell-derived nanovesicle described herein comprises a rhabdovirus glycoprotein from YBV-G. In some embodiments, the YBV-G comprises the sequence SEQ ID NO: 66. In some embodiments, the YBV-G consists of the sequence SEQ ID NO: 66.

In some embodiments, a recombinant pseudotyped virus or cell-derived nanovesicle of the present disclosure comprises a glycoprotein from a Yinshui Bat virus (YSBV-G). In some embodiments, the YSBV-G comprises the amino acid sequence of SEQ ID NO: 67 or 92, or a functional fragment or derivative thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity with SEQ ID NO: 67 or 92. In certain embodiments, the nucleotide sequence that encodes the YSBV-G comprises the nucleotide sequence that encodes the amino acid sequence of SEQ ID NO: 67 or 92, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 67 or 92. In certain embodiments, the YSBV-G comprises the amino acid sequence of SEQ ID NO: 67 or 92.

In some embodiments, a recombinant pseudotyped virus or cell-derived nanovesicle described herein comprises a rhabdovirus glycoprotein from YSBV-G. In some embodiments, the YSBV-G comprises the sequence SEQ ID NO: 67. In some embodiments, the YSBV-G consists of the sequence SEQ ID NO: 67.

In some embodiments, a recombinant pseudotyped virus or cell-derived nanovesicle of the present disclosure comprises a glycoprotein from a Kimberley virus (KIMV-G). In some embodiments, the KIMV-G comprises the amino acid sequence of SEQ ID NO: 68 or 96, or a functional fragment or derivative thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity with SEQ ID NO: 68 or 96. In certain embodiments, the nucleotide sequence that encodes the KIMV-G comprises the nucleotide sequence that encodes the amino acid sequence of SEQ ID NO: 68 or 96, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 68 or 96. In certain embodiments, the KIMV-G comprises the amino acid sequence of SEQ ID NO: 68 or 96.

In some embodiments, a recombinant pseudotyped virus or cell-derived nanovesicle described herein comprises a rhabdovirus glycoprotein from KIMV-G. In some embodiments, the KIMV-G comprises the sequence SEQ ID NO: 68. In some embodiments, the KIMV-G consists of the sequence SEQ ID NO: 68.

In some embodiments, a recombinant pseudotyped virus or cell-derived nanovesicle of the present disclosure comprises a glycoprotein from a Kanyawara virus (KYAV-G). In some embodiments, the KYAV-G comprises the amino acid sequence of SEQ ID NO: 69 or 98, or a functional fragment or derivative thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity with SEQ ID NO: 69 or 98. In certain embodiments, the nucleotide sequence that encodes the KYAV-G comprises the nucleotide sequence that encodes the amino acid sequence of SEQ ID NO: 69 or 98, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 69 or 98. In certain embodiments, the KYAV-G comprises the amino acid sequence of SEQ ID NO: 69 or 98.

In some embodiments, a recombinant pseudotyped virus or cell-derived nanovesicle described herein comprises a rhabdovirus glycoprotein from KYAV-G. In some embodiments, the KYAV-G comprises the sequence SEQ ID NO: 69. In some embodiments, the KYAV-G consists of the sequence SEQ ID NO: 69.

In some embodiments, a recombinant pseudotyped virus or cell-derived nanovesicle of the present disclosure comprises a glycoprotein from a La Joya virus (LJV-G). In some embodiments, the LJV-G comprises the amino acid sequence of SEQ ID NO: 70 or 99, or a functional fragment or derivative thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity with SEQ ID NO: 70 or 99. In certain embodiments, the nucleotide sequence that encodes the LJV-G comprises the nucleotide sequence that encodes the amino acid sequence of SEQ ID NO: 70 or 99, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 70 or 99. In certain embodiments, the LJV-G comprises the amino acid sequence of SEQ ID NO: 70 or 99.

In some embodiments, a recombinant pseudotyped virus or cell-derived nanovesicle described herein comprises a rhabdovirus glycoprotein from LJV-G. In some embodiments, the LJV-G comprises the sequence SEQ ID NO: 70. In some embodiments, the LJV-G consists of the sequence SEQ ID NO: 70.

In some embodiments, a recombinant pseudotyped virus or cell-derived nanovesicle of the present disclosure comprises a glycoprotein from a Mosquiero virus (MQOV-G). In some embodiments, the MQOV-G comprises the amino acid sequence of SEQ ID NO: 71 or 100, or a functional fragment or derivative thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity with SEQ ID NO: 71 or 100. In certain embodiments, the nucleotide sequence that encodes the MQOV-G comprises the nucleotide sequence that encodes the amino acid sequence of SEQ ID NO: 71 or 100, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 71 or 100. In certain embodiments, the MQOV-G comprises the amino acid sequence of SEQ ID NO: 71 or 100.

In some embodiments, a recombinant pseudotyped virus or cell-derived nanovesicle described herein comprises a rhabdovirus glycoprotein from MQOV-G. In some embodiments, the MQOV-G comprises the sequence SEQ ID NO: 71. In some embodiments, the MQOV-G consists of the sequence SEQ ID NO: 71.

In some embodiments, a recombinant pseudotyped virus or cell-derived nanovesicle of the present disclosure comprises a glycoprotein from a Parry Creek virus (PCV-G). In some embodiments, the PCV-G comprises the amino acid sequence of SEQ ID NO: 72 or 101, or a functional fragment or derivative thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity with SEQ ID NO: 72 or 101. In certain embodiments, the nucleotide sequence that encodes the PCV-G comprises the nucleotide sequence that encodes the amino acid sequence of SEQ ID NO: 72 or 101, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 72 or 101. In certain embodiments, the PCV-G comprises the amino acid sequence of SEQ ID NO: 72 or 101.

In some embodiments, a recombinant pseudotyped virus or cell-derived nanovesicle described herein comprises a rhabdovirus glycoprotein from PCV-G. In some embodiments, the PCV-G comprises the sequence SEQ ID NO: 72. In some embodiments, the PCV-G consists of the sequence SEQ ID NO: 72.

In some embodiments, a recombinant pseudotyped virus or cell-derived nanovesicle of the present disclosure comprises a glycoprotein from a Bas Congo virus (BASV-G). In some embodiments, the BASV-G comprises the amino acid sequence of SEQ ID NO: 73 or 102, or a functional fragment or derivative thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity with SEQ ID NO: 73 or 102. In certain embodiments, the nucleotide sequence that encodes the BASV-G comprises the nucleotide sequence that encodes the amino acid sequence of SEQ ID NO: 73 or 102, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 73 or 102. In certain embodiments, the BASV-G comprises the amino acid sequence of SEQ ID NO: 73 or 102.

In some embodiments, a recombinant pseudotyped virus or cell-derived nanovesicle described herein comprises a rhabdovirus glycoprotein from BASV-G. In some embodiments, the BASV-G comprises the sequence SEQ ID NO: 73. In some embodiments, the BASV-G consists of the sequence SEQ ID NO: 73.

In some embodiments, a recombinant pseudotyped virus or cell-derived nanovesicle of the present disclosure comprises a glycoprotein from a Bovine Ephemeral fever virus (BEFV-G). In some embodiments, the BEFV-G comprises the amino acid sequence of SEQ ID NO: 74 or 103, or a functional fragment or derivative thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity with SEQ ID NO: 74 or 103. In certain embodiments, the nucleotide sequence that encodes the BEFV-G comprises the nucleotide sequence that encodes the amino acid sequence of SEQ ID NO: 74 or 103, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 74 or 103. In certain embodiments, the BEFV-G comprises the amino acid sequence of SEQ ID NO: 74 or 103.

In some embodiments, a recombinant pseudotyped virus or cell-derived nanovesicle described herein comprises a rhabdovirus glycoprotein from BEFV-G. In some embodiments, the BEFV-G comprises the sequence SEQ ID NO: 74. In some embodiments, the BEFV-G consists of the sequence SEQ ID NO: 74.

In some embodiments, a recombinant pseudotyped virus or cell-derived nanovesicle of the present disclosure comprises a glycoprotein from a Curionopolis virus (CURV-G). In some embodiments, the CURV-G comprises the amino acid sequence of SEQ ID NO: 75 or 104, or a functional fragment or derivative thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity with SEQ ID NO: 75 or 104. In certain embodiments, the nucleotide sequence that encodes the CURV-G comprises the nucleotide sequence that encodes the amino acid sequence of SEQ ID NO: 75 or 104, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 75 or 104. In certain embodiments, the CURV-G comprises the amino acid sequence of SEQ ID NO: 75 or 104.

In some embodiments, a recombinant pseudotyped virus or cell-derived nanovesicle described herein comprises a rhabdovirus glycoprotein from CURV-G. In some embodiments, the CURV-G comprises the sequence SEQ ID NO: 75. In some embodiments, the CURV-G consists of the sequence SEQ ID NO: 75.

In some embodiments, a recombinant pseudotyped virus or cell-derived nanovesicle of the present disclosure comprises a glycoprotein from a Drosophila melanogaster sigmavirus (DMelSV-G). In some embodiments, the DMelSV-G comprises the amino acid sequence of SEQ ID NO: 76 or 105, or a functional fragment or derivative thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity with SEQ ID NO: 76 or 105. In certain embodiments, the nucleotide sequence that encodes the DMelSV-G comprises the nucleotide sequence that encodes the amino acid sequence of SEQ ID NO: 76 or 105, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 76 or 105. In certain embodiments, the DMelSV-G comprises the amino acid sequence of SEQ ID NO: 76 or 105.

In some embodiments, a recombinant pseudotyped virus or cell-derived nanovesicle described herein comprises a rhabdovirus glycoprotein from DMelSV-G. In some embodiments, the DMelSV-G comprises the sequence SEQ ID NO: 76. In some embodiments, the DMelSV-G consists of the sequence SEQ ID NO: 76.

In some embodiments, a recombinant pseudotyped virus or cell-derived nanovesicle of the present disclosure comprises a glycoprotein from a Niakha virus (NIAV-G). In some embodiments, the NIAV-G comprises the amino acid sequence of SEQ ID NO: 77 or 106, or a functional fragment or derivative thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity with SEQ ID NO: 77 or 106. In certain embodiments, the nucleotide sequence that encodes the NIAV-G comprises the nucleotide sequence that encodes the amino acid sequence of SEQ ID NO: 77 or 106, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 77 or 106. In certain embodiments, the NIAV-G comprises the amino acid sequence of SEQ ID NO: 77 or 106.

In some embodiments, a recombinant pseudotyped virus or cell-derived nanovesicle described herein comprises a rhabdovirus glycoprotein from NIAV-G. In some embodiments, the NIAV-G comprises the sequence SEQ ID NO: 77. In some embodiments, the NIAV-G consists of the sequence SEQ ID NO: 77.

In some embodiments, a recombinant pseudotyped virus or cell-derived nanovesicle of the present disclosure comprises a glycoprotein from a Puerto almandras virus (PTAMV-G). In some embodiments, the PTAMV-G comprises the amino acid sequence of SEQ ID NO: 78 or 107, or a functional fragment or derivative thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity with SEQ ID NO: 78 or 107. In certain embodiments, the nucleotide sequence that encodes the PTAMV-G comprises the nucleotide sequence that encodes the amino acid sequence of SEQ ID NO: 78 or 107, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 78 or 107. In certain embodiments, the PTAMV-G comprises the amino acid sequence of SEQ ID NO: 78 or 107.

In some embodiments, a recombinant pseudotyped virus or cell-derived nanovesicle described herein comprises a rhabdovirus glycoprotein from PTAMV-G. In some embodiments, the PTAMV-G comprises the sequence SEQ ID NO: 78. In some embodiments, the PTAMV-G consists of the sequence SEQ ID NO: 78.

In some embodiments, a recombinant pseudotyped virus or cell-derived nanovesicle of the present disclosure comprises a glycoprotein from a Tupaia virus (TUPTV-G). In some embodiments, the TUPTV-G comprises the amino acid sequence of SEQ ID NO: 79 or 108, or a functional fragment or derivative thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity with SEQ ID NO: 79 or 108. In certain embodiments, the nucleotide sequence that encodes the TUPTV-G comprises the nucleotide sequence that encodes the amino acid sequence of SEQ ID NO: 79 or 108, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 79 or 108. In certain embodiments, the TUPTV-G comprises the amino acid sequence of SEQ ID NO: 79 or 108.

In some embodiments, a recombinant pseudotyped virus or cell-derived nanovesicle described herein comprises a rhabdovirus glycoprotein from TUPTV-G. In some embodiments, the TUPTV-G comprises the sequence SEQ ID NO: 79. In some embodiments, the TUPTV-G consists of the sequence SEQ ID NO: 79.

In some embodiments, a recombinant pseudotyped virus or cell-derived nanovesicle described herein can comprise a fragment of a rhabdovirus glycoprotein described herein, and the cytoplasmic tail of the rhabdovirus glycoprotein has been removed or truncated, and/or optionally replaced with another sequence.

In various embodiments of the recombinant pseudotyped virus or cell-derived nanovesicle described herein, the glycoprotein fragment has a truncated cytoplasmic tail. For example, the cytoplasmic tail of the rhabdovirus glycoprotein may be truncated by 2 to 80, 3 to 75, 4 to 70, 5 to 65, 6 to 60, 7 to 55, 8 to 50, 9 to 45, 10 to 40, 11 to 35, 12 to 30, 13 to 25, or 14 to 20, or 15 to 35 amino acids from the C-terminus. In certain embodiments, the cytoplasmic tail of the rhabdovirus glycoprotein which has been truncated may be truncated 10 to 40 amino acids from the C-terminus. In some embodiments, the cytoplasmic tail of the rhabdovirus glycoprotein may be truncated by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80, or more, amino acids from the C-terminus. In some embodiments, the cytoplasmic tail of the rhabdovirus glycoprotein may be truncated by 30 amino acids from the C-terminus.

In some embodiments, the cytoplasmic tail of a rhabdovirus glycoprotein can be truncated by up to 40 amino acids from the C-terminus. In some embodiments, the rhabdovirus glycoprotein can be truncated by 10 to 40 amino acids from the C-terminus. In some embodiments, the rhabdovirus glycoprotein can be truncated by 30 amino acids from the C-terminus.

In some embodiments, the glycoprotein fragment included in a recombinant pseudotyped virus or cell-derived nanovesicle described herein may further comprise a cytoplasmic tail from VSV-G, or a fragment or derivative thereof. A non-limiting example of a cytoplasmic tail is the amino acid sequence of CIKLKHTKKRQIYTDIEMNRLGK (SEQ ID NO: 16). In some embodiments, the cytoplasmic tail of a rhabdovirus glycoprotein described herein comprises the amino acid sequence of SEQ ID NO: 16, or a fragment or derivative thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity with SEQ ID NO: 16. In certain embodiments, the nucleotide sequence that encodes the cytoplasmic tail of the rhabdovirus glycoprotein comprises the nucleotide sequence that encodes the amino acid sequence of SEQ ID NO: 16, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 16. In certain embodiments, the cytoplasmic tail of the rhabdovirus glycoprotein comprises the amino acid sequence of SEQ ID NO: 16.

In various embodiments, the recombinant pseudotyped virus described herein can be an oncolytic virus. Non-limiting examples of oncolytic viruses include an adenovirus, a herpes virus, a pox virus, retrovirus, a paramyxovirus or a reovirus, or any species or strain within these larger groups. A virus disclosed herein may be unaltered from the parental virus species (i.e., wild-type, WT), or with gene modifications, e.g., gene additions.

Oncolytic virus therapy is an anti-cancer approach which uses viruses to selectively replicate and kill cancer cells without harming healthy cells. Such viruses are a powerful platform for oncolytic immunotherapy, which involves both direct tumor cell lysis and the induction of immune responses against tumor antigens. In general, viruses can enter cells at high efficiency. Viral genes are subsequently expressed, and the virus replicates. Viruses may also be used as delivery vehicles to express heterologous genes inserted into the viral genome in infected cells, and thus viruses such as oncolytic viruses can deliver encoded molecules which may help, for example, to selectively hone the systemic anti-tumor immune response.

An oncolytic virus is a virus capable of infecting and replicating within tumor cells, such that the tumor cells are broken down, e.g., lysed, and killed. Therefore, the oncolytic virus of the disclosure may be replication competent. In some embodiments, the virus may be selectively replication competent in a specific tissue, for example, a tumor tissue. By way of a non-limiting example, a virus is selectively replication competent in a tumor tissue if it replicates more effectively in a tumor tissue than in a non-tumor tissue. The ability of a virus to replicate in different tissue types may be determined using, for example, without limitation, real-time quantitative reverse transcription polymerase chain reaction (qRT-PCR), electrophoresis, chromatography, flow cytometry, tunable resistive pulse sensing (TRPS), microscopy techniques (e.g., fluorescence microscopy, electron microscopy, including transmission electron microscopy), fluorescence in situ hybridization (FISH), infectivity assays, enzyme-linked immunosorbent assays (ELISAs), fluorescent-tagged antibody assays, and precipitation assays, loop-mediated amplification (LAMP) assay, plaque assay, focus forming assays (FFAs), 50% tissue culture infective dose (TCID50) assay, virus neutralization assays, homogenous virus assays, e.g., fluorescence polarization-based assays, AlphaScreen®/AlphaLISA® technologies, proximity-based assays, e.g. FRET, BRET, TR-FRET, time-resolved fluorescence (TRF), RNA dependent RNA polymerase assays, fluorescence in vitro and high throughput assays, cell-based microplate assays, protein assays (e.g., bicinchoninic acid assay, single radial immunodiffusion assay), and the like.

In some embodiments, oncolytic effects of the oncolytic virus disclosed herein may rely on the virus replicating in and killing infected cells. Progeny virions may then proceed to infect and kill other tumor cells, thereby spreading within the tumor. Hence, the ability of the oncolytic virus to effectively kill tumor cells and spread within tumors results in optimal anti-tumor effects. Efficient spread and virus replication is associated with lysis of tumor cells, a process which may also maximize the quantity of tumor antigen released and therefore maximize the potency of the induced anti-tumor immune response.

In some embodiments, an oncolytic virus disclosure herein may be replication deficient. For instance, in such embodiments, a replication deficient oncolytic virus may comprise an incomplete genome of the virus form which they are derived. As a non-limiting example, in some embodiments, when an oncolytic virus described herein may comprise, e.g., a LV described herein, the LV may not comprise the genetic information of a gag, env, and/or pol genes which may be involved in the assembly of the viral particle, and can be a minimal requirement for successful replication of a LV.

An oncolytic virus disclosed herein may be derived from any enveloped virus. Non-limiting examples of enveloped viruses from which the recombinant virus may be derived include, without limitation, retroviruses (e.g., rous sarcoma virus, human and bovine T-cell leukemia virus (HTLV and BLV)), lentiviruses (e.g., human and simian immunodeficiency viruses (HIV and SIV), Mason-Pfizer monkey virus), foamy viruses (e.g., Human Foamy Virus (HFV)), herpes viruses (herpes simplex virus (HSV), varicella-zoster virus, VZVEBV, HCMV, HHV), hantaviruses, pox viruses (e.g., vertebrate and avian poxviruses, vaccinia viruses), orthomyxoviruses (e.g., influenza A, influenza B, influenza C viruses), paramyxoviruses (e.g., parainfluenza virus, respiratory syncytial virus, Sendai virus, mumps virus, measles and measles-like viruses), rhabdoviruses (e.g., vesicular stomatitis virus, rubella virus, rabies virus), coronaviruses (e.g., SARS, MERS), flaviviruses (e.g., Marburg virus, Reston virus, Ebola virus), alphaviruses (e.g., Sindbis virus), bunyaviruses, arenaviruses (e.g., LCMV, GTOV, JUNV, LASV, LUJV, MACV, SABV, WWAV), iridoviruses, and hepadnaviruses.

In some embodiments, a virus of the present disclosure (e.g., a recombinant pseudotyped virus described herein) can be derived from a lentivirus or a retrovirus. Compared to other gene transfer systems, lentiviral and retroviral vectors can offer a wide range of advantages, including, for example, their ability to transduce a variety of cell types, to stably integrate transferred genetic material into the genome of the targeted host cell, and/or to express the transduced gene at significant levels. Vectors derived from the gamma-retroviruses, for example, the murine leukemia virus (MLV), have been used in clinical gene therapy trials.

In some embodiments, the oncolytic virus may be derived from a rhabdovirus. In some embodiments, the oncolytic virus may comprise a rhabdovirus genome. Non-limiting examples of rhabdoviruses that can be used in the present disclosure include rabies, cytolabudoviruses, dicholabdoviruses, ephemeraviruses, lyssaviruses, nobilabdoviruses, and vesiculoviruses. In some embodiments, the rhabdovirus is a vesiculovirus including, without limitation, a vesicular stomatitis virus (VSV).

### Vesicular Stomatitis Virus (VSV)

In certain aspects, the recombinant pseudotyped virus of the present disclosure may comprise a vesiculovirus genome, for example, a VSV genome. The VSV genome may comprise genes which may encode a VSV nucleoprotein (N) polypeptide, a VSV phosphoprotein (P) polypeptide, a VSV matrix (M) polypeptide, a fusogenic polypeptide, a VSV large protein (L) polypeptide, or functional fragments or derivatives thereof. The sequences disclosed herein with respect to the recombinant virus disclosed herein, e.g., VSV, are incorporated into a plasmid coding for the positive sense cDNA of the viral genome allowing generation, for example, of the negative sense genome of the recombinant viruses, e.g., VSVs. Thus, a nucleic acid sequence that encodes a VSV polypeptide, for example, can refer to a nucleotide sequence, e.g., an RNA sequence, that is the template for the positive sense transcript that encodes (e.g., via direct translation) that polypeptide.

In some embodiments, the VSV genome comprises one or more genes encoding a VSV nucleoprotein (N) polypeptide, a VSV phosphoprotein (P) polypeptide, a VSV matrix (M) polypeptide, a fusogenic polypeptide, and a VSV large protein (L) polypeptide, or a functional fragment or derivative thereof.

In some embodiments, the VSV genome may comprise genes which may encode a fusogenic polypeptide, wherein the fusogenic polypeptide is a glycoprotein (G) polypeptide from a different rhabdovirus as described above.

For instance, in some cases, the fusogenic polypeptide can be a glycoprotein (G) polypeptide derived from a vesicular stomatitis virus glycoprotein (VSV-G). In some embodiments, the VSV-G comprises the amino acid sequence of SEQ ID NO: 8 or 80, or a functional fragment or derivative thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity with SEQ ID NO: 8 or 80. In certain embodiments, the nucleotide sequence that encodes the VSV-G comprises the nucleotide sequence that encodes the amino acid sequence of SEQ ID NO: 8 or 80, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 8 or 80. In certain embodiments, the VSV-G comprises the amino acid sequence of SEQ ID NO: 8 or 80.

In some embodiments, a fusogenic polypeptide described herein comprises a rhabdovirus glycoprotein from VSV-G. In some embodiments, the VSV-G comprises the sequence SEQ ID NO: 8. In some embodiments, the VSV-G consists of the sequence SEQ ID NO: 8.

Another non-limiting example of a vesicular stomatitis virus glycoprotein (VSV-G) is described in U.S. Patent Publication No. 2020/0216502, the content of which is incorporated herein by reference in its entirety for all purposes. As an example, without limitation, a VSV-G, or functional fragment or derivative thereof described herein may comprise the amino acid sequence of SEQ ID NO: 157, or a functional fragment or derivative thereof.

In certain aspects, the present disclosure provides a recombinant virus, for example, a recombinant replication-competent virus, e.g., VSV, comprising an RNA molecule. The RNA molecule may comprise, or consist essentially of, a nucleotide sequence that is a template for a positive sense transcript encoding a VSV nucleoprotein (N) polypeptide or a functional fragment or derivative thereof, a nucleotide sequence that is a template for a positive sense transcript encoding a VSV phosphoprotein (P) polypeptide or a functional fragment or derivative thereof, a nucleotide sequence that is a template for a positive sense transcript encoding a VSV matrix (M) polypeptide or a functional fragment or derivative thereof, a nucleotide sequence that is a template for a positive sense transcript encoding a fusogenic polypeptide (e.g., a glycoprotein (G) polypeptide from a different rhabdovirus as described above) or a functional fragment or derivative thereof, a nucleotide sequence that is a template for a positive sense transcript encoding a VSV large protein (L) polypeptide (polymerase) or a functional fragment or derivative thereof. In some embodiments, the RNA molecule may comprise a nucleotide sequence that is a template for a positive sense transcript encoding a transgene.

In certain aspects, the present disclosure provides a recombinant virus, for example, a recombinant replication-competent virus, e.g., VSV, comprising an RNA molecule. The RNA molecule may comprise, or consist essentially of, a nucleotide sequence that is a template for a positive sense transcript encoding a VSV nucleoprotein (N) polypeptide or a functional fragment or derivative thereof, a nucleotide sequence that is a template for a positive sense transcript encoding a VSV phosphoprotein (P) polypeptide or a functional fragment or derivative thereof, a nucleotide sequence that is a template for a positive sense transcript encoding a VSV matrix (M) polypeptide or a functional fragment or derivative thereof, a nucleotide sequence that is a template for a positive sense transcript encoding a fusogenic polypeptide (e.g., a glycoprotein (G) polypeptide from a different rhabdovirus as described above) or a functional fragment or derivative thereof, a nucleotide sequence that is a template for a positive sense transcript encoding a VSV large protein (L) polypeptide (polymerase) or a functional fragment or derivative thereof.

Without wishing to be bound by theory, the genome of vesicular stomatitis virus (VSV), considered a prototypic (exemplary) rhabdovirus, comprises 11,161 nucleotides of negative-sense RNA. This RNA is tightly encapsidated by the viral nucleocapsid (N) polypeptide and forms the template for the RNA-dependent RNA polymerase (RdRP), the viral components being a 241-kDa large protein (L) polypeptide and a phosphoprotein (P) polypeptide. The RdRP uses the RNA (viral) genome as a template for two reactions: (i) transcription of a short leader RNA (Le+) and 5 mRNAs that encode nucleoprotein (N) polypeptide, phosphoprotein (P) polypeptide, matrix (M) polypeptide, glycoprotein (G) polypeptide, and large protein (L) polypeptide; and (ii) replication to yield full-length antigenomic and then genomic strands.

The VSV genome can be genetically modified to include one or more mutations or “mutation classes” in the genome. “Mutation class”, “mutation classes” or “classes of mutation” are used interchangeably herein. Example mutation classes include, but are not limited to, a VSV temperature-sensitive N gene mutation, a temperature-sensitive L gene mutation, a point mutation, a G-stem mutation, a non-cytopathic M gene mutation, a gene shuffling or rearrangement mutation, a truncated G gene mutation, an ambisense RNA mutation, a G gene insertion mutation, a gene deletion mutation and the like. Mutations can be insertions, deletions, substitutions, gene rearrangement, or shuffling modifications.

Some mutations may attenuate the infectivity, virulence or pathogenic effects of VSV. The attenuation can be additive or synergistic. With synergistic attenuation, the level of VSV attenuation is greater than additive. Synergistic attenuation of VSV can arise from combining at least two classes of mutation in the same VSV genome, thereby resulting in a reduction of VSV pathogenicity much greater than an additive attenuation level observed for each VSV mutation class alone. A synergistic attenuation of VSV can provide for an LD₅₀ at least greater than the additive attenuation level observed for each mutation class alone (i.e., the sum of the two mutation classes), where attenuation levels (i.e., the LD₅₀) are determined in a small animal neurovirulence model.

The matrix (M) polypeptide of rhabdoviruses including, such as VSVs, are small (approximately 20-25 kDa) multifunctional proteins that play a role in virus assembly, maturation, and budding (replication). The M protein also modulates the production of host and virus proteins, e.g., promoting viral egress from the host cell and cell death. From a structural standpoint, the M protein forms a layer between the glycoprotein- (G-) containing outer membrane and the nucleocapsid core which includes the virus nucleoprotein (N), large (L), phosphoprotein (P) and RNA (viral) genome.

The VSV M gene encodes the virus matrix (M) protein, and two smaller in-frame polypeptides (M2 and M3). The M2 and M3 polypeptides can be translated from the same open reading frame (ORF) as the M protein and lack the first 33 and 51 amino acids, respectively. A recombinant VSV vector comprising non-cytopathic M gene mutations (i.e., VSV vectors that also do not express M2 and M3 proteins) can be generated and can further comprise one or more additional mutation(s) thereby resulting in a VSV vector that was highly attenuated in cell culture and in animals.

In certain embodiments, the recombinant VSV described herein may comprise a non-cytopathic mutation in the M gene. The VSV (Indiana serotype) M gene encodes a 229 amino acid M (matrix) protein in which the first thirty amino acids of the NH2-terminus comprise a proline-rich PPPY (SEQ ID NO: 227) (PY) motif. The PY motif of VSV M protein is located at amino acid positions 24-27 in both VSV Indiana (Genbank Accession Number X04452) and New Jersey (Genbank Accession Number M14553) serotypes. The VSV may comprise mutations in the PY motif (e.g., APPY (SEQ ID NO: 228), AAPY (SEQ ID NO: 229), PPAY (SEQ ID NO: 230), APPA (SEQ ID NO: 231), AAPA (SEQ ID NO: 232) and PPPA (SEQ ID NO: 233)). The VSV can comprise any of various amino acid mutations (e.g., deletions, substitutions, insertions, etc.) into the M protein PSAP (SEQ ID NO: 234) (PS) motif. These and other mutations in the PY motif may be effective to reduce virus yield by blocking a late stage in virus budding.

The recombinant VSV described herein may comprise one or more M gene mutations. Non-limiting examples of M protein mutations include, e.g., a glycine changed to a glutamic acid at position (21), a leucine changed to a phenylalanine at position (111), a methionine changed to an arginine at position (51), a glycine changed to a glutamic acid at position (22), a methionine changed to an arginine at position (48), a leucine changed to a phenylalanine at position (110), a methionine changed to an alanine at position (51), and a methionine changed to an alanine at position (33). In various embodiments of the methods described herein, the genome of the recombinant VSV encodes a mutant VSV matrix M protein comprising the M51R variant M protein. The M51R mutation can eliminate the ability of the M protein to block cellular nucleo-cytoplasmic transport, and thus can substantially attenuate VSV infectivity.

In some embodiments, the VSV matrix (M) polypeptide is a wild-type VSV M polypeptide.

In some embodiments, the VSV matrix (M) polypeptide is a mutant VSV M polypeptide. In some embodiments, the mutant VSV M polypeptide may comprise a mutation at methionine (M) 51 (M51R). In some embodiments, the mutation may comprise a substitution from methionine (M) to arginine (R). In some embodiments, the mutant VSV M polypeptide may comprise a deletion at methionine (M) 51 (ΔM51).

In some embodiments, the wild-type VSV matrix (M) polypeptide comprises the amino acid sequence of SEQ ID NO: 138, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 138. In certain embodiments, the nucleotide sequence that encodes the wild-type VSV matrix (M) polypeptide comprises the nucleotide sequence that encodes the amino acid sequence of SEQ ID NO: 138, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 138. In certain embodiments, the nucleotide sequence that encodes the wild-type VSV matrix (M) polypeptide comprises the nucleotide sequence of SEQ ID NO: 139, or a nucleotide sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 139. In certain embodiments, the wild-type VSV matrix (M) polypeptide comprises the amino acid sequence of SEQ ID NO: 138. In certain embodiments, the nucleotide sequence that encodes the wild-type VSV matrix (M) polypeptide comprises the nucleotide sequence of SEQ ID NO: 139.

In some embodiments, the VSV M polypeptide comprises the amino acid sequence of SEQ ID NO: 138 or an amino acid sequence that has at least 80% sequence identity to SEQ ID NO: 138.

In some embodiments, the mutant VSV matrix (M) polypeptide M51R comprises the amino acid sequence of SEQ ID NO: 140, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 140. In certain embodiments, the nucleotide sequence that encodes the mutant VSV matrix (M) polypeptide M51R comprises the nucleotide sequence that encodes the amino acid sequence of SEQ ID NO: 140, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 140. In certain embodiments, the nucleotide sequence that encodes the mutant VSV matrix (M) polypeptide M51R comprises the nucleotide sequence of SEQ ID NO: 141, or a nucleotide sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 141. In certain embodiments, the mutant VSV matrix (M) polypeptide M51R comprises the amino acid sequence of SEQ ID NO: 140. In certain embodiments, the nucleotide sequence that encodes the mutant VSV matrix (M) polypeptide M51R comprises the nucleotide sequence of SEQ ID NO: 141.

In some embodiments, the VSV M polypeptide comprises the amino acid sequence of SEQ ID NO: 140 or an amino acid sequence that has at least 80% sequence identity to SEQ ID NO: 140.

In some embodiments, the mutant VSV matrix (M) polypeptide ΔM51 comprises the amino acid sequence of SEQ ID NO: 142, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 142. In certain embodiments, the nucleotide sequence that encodes the mutant VSV matrix (M) polypeptide ΔM51 comprises the nucleotide sequence that encodes the amino acid sequence of SEQ ID NO: 142, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 142. In certain embodiments, the nucleotide sequence that encodes the mutant VSV matrix (M) polypeptide ΔM51 comprises the nucleotide sequence of SEQ ID NO: 143, or a nucleotide sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 143. In certain embodiments, the mutant VSV matrix (M) polypeptide ΔM51 comprises the amino acid sequence of SEQ ID NO: 142. In certain embodiments, the nucleotide sequence that encodes the mutant VSV matrix (M) polypeptide ΔM51 comprises the nucleotide sequence of SEQ ID NO: 143.

In some embodiments, the VSV M polypeptide comprises the amino acid sequence of SEQ ID NO: 142 or an amino acid sequence that has at least 80% sequence identity to SEQ ID NO: 142.

In some embodiments, the VSV genome, provided herein that encode a VSV N polypeptide, a VSV P polypeptide, a VSV M polypeptide, and a VSV L polypeptide can be from a VSV Indiana strain as set forth in GenBank® Accession Nos. NC_001560 (GI No. 9627229) or can be from a VSV New Jersey strain.

Any appropriate method may be used to insert a nucleic acid disclosed herein (e.g., nucleotide sequence that is a template for a positive sense transcript encoding a polypeptide such as, but not limited to, a virus polypeptide, and/or a nucleotide sequence that is a template for a positive sense transcript encoding a virus polypeptide) into the genome of a recombinant virus of the present disclosure. For example, any of various nucleic acid generation and/or amplification techniques via, e.g., polymerase chain reaction (PCR) and the like, including from a plasmid, gene excision, reverse genetics, cloning, and/or subcloning steps, virus preparation, growth, propagation, and/or recovery, and/or cell culture approaches comprising, e.g., infection, transduction and/or transfection, or combinations thereof, may be used to insert nucleic acid into the genome of a recombinant virus. Any appropriate method may be used to identify recombinant virus-containing a nucleic acid molecule disclosed herein. Non-limiting examples of such methods include and nucleic acid hybridization techniques including Northern and Southern analysis, and polymerase chain reaction (PCR). In some embodiments, biochemical techniques and/or immunohistochemistry may be used to determine if a recombinant virus contains a specific nucleic acid molecule by detecting the expression of a polypeptide encoded by that specific nucleic acid molecule.

### Molecular Cargoes

In certain aspects, the present disclosure provides a recombinant pseudotyped virus and/or cell-derived nanovesicle described herein further comprising a molecular cargo. Non-limiting examples of a molecular cargo are a transgene encoding a therapeutic protein, a suicide gene, a toxic protein or peptide, an antibody or a fragment thereof, a chimeric antigen receptor (CAR), a T cell receptor (TCR), a gene editing system or a component(s) thereof, an antisense oligonucleotide, a ribozyme, or an RNAi molecule. In some embodiments, the molecular cargo can be a gene editing ribonucleoprotein complex or a component(s) thereof. In various embodiments, the molecular cargo can be, for example, Cas9 protein complexed with a guide RNA (gRNA) specific to a gene of interest.

In some embodiments, the molecular cargo comprises a transgene encoding a therapeutic protein, a toxic protein or peptide, an antibody or a fragment thereof, a chimeric antigen receptor (CAR), a T cell receptor (TCR), a gene editing system or a component(s) thereof, an antisense oligonucleotide, a ribozyme, or an RNAi molecule.

In some embodiments, the molecular cargo comprises a transgene encoding a therapeutic protein comprising, for example an antibody or antigen-binding fragment thereof, TCR, and/or CAR.

In some embodiments, the molecular cargo of the disclosure can be a transgene encoding an antibody or an antigen-binding fragment thereof. Antibodies or antigen-binding fragments thereof encompass derivatives, functional equivalents, and homologues of antibodies, humanized antibodies, including any polypeptide comprising an immunoglobulin binding domain, whether natural or wholly or partially synthetic and any polypeptide or protein having a binding domain which is, or is homologous to, an antibody binding domain. Chimeric molecules comprising an immunoglobulin binding domain, or equivalent, fused to another polypeptide are therefore included. A humanized antibody may be a modified antibody having the variable regions of a non-human, e.g., murine, antibody, and the constant region of a human antibody. Examples of antibodies are the immunoglobulin isotypes (e.g., IgG, IgE, IgM, IgD and IgA) and their isotypic subclasses; or fragments that comprise an antigen binding domain such as Fab, scFv, Fv, dAb, Fd; and diabodies. Antibodies may be polyclonal or monoclonal. A monoclonal antibody may be referred to herein as “mAb”.

In some embodiments, the antibody encoded by the transgene can be a multispecific antibody. In some embodiments, the antibody is a bispecific antibody. The bispecific antibody may comprise a second targeting moiety that targets to the desired cell or tissue, e.g., a desired antigen associated cancer.

It is possible to take an antibody, for example a mAb, and use recombinant DNA technology to produce other antibodies or chimeric molecules which retain the specificity of the original antibody. Such techniques may involve introducing DNA encoding the immunoglobulin variable region, or the complementary determining regions (CDRs), of an antibody to the constant regions, or constant regions plus framework regions, of a different immunoglobulin. A hybridoma (or other cell that produces antibodies) may be subject to genetic mutation or other changes, which may or may not alter the binding specificity of antibodies produced.

It has been shown that fragments of a whole antibody can perform the function of binding antigens. Examples of binding fragments are (i) the Fab fragment consisting of V_{L}, V_{H}, C_{L} and CH1 domains; (ii) the Fd fragment consisting of the V_{H} and CH1 domains; (iii) the Fv fragment consisting of the V_{L} and V_{H} domains of a single antibody; (iv) the dAb fragment which consists of a V_{H} domain; (v) isolated CDR regions; (vi) F(ab′)2 fragments, a bivalent fragment comprising two linked Fab fragments; (vii) single chain Fv molecules (scFv), wherein a V_{H} domain and a V_{L} domain are linked by a peptide linker which allows the two domains to associate to form an antigen binding site; (viii) bispecific single chain Fv dimers, (ix) “diabodies”, multivalent or multispecific fragments constructed by gene fusion, and (x) VHH or VNAR antibodies, also known as single-domain antibodies or nanobodies (Nb), which may be derived from heavy-chain antibodies from e.g., dromedaries, camels, llamas, alpacas, or sharks.

Diabodies are multimers of polypeptides, each polypeptide comprising a first domain comprising a binding region of an immunoglobulin light chain and a second domain comprising a binding region of an immunoglobulin heavy chain, the two domains being linked (e.g., by a peptide linker) but unable to associate with each other to form an antigen binding site: antigen binding sites are formed by the association of the first domain of one polypeptide within the multimer with the second domain of another polypeptide within the multimer. Where bispecific antibodies are to be used, these may be conventional bispecific antibodies, which can be manufactured in a variety of ways, e.g., prepared chemically or from hybrid hybridomas, or may be any of the bispecific antibody fragments mentioned above. It may be preferable to use scFv dimers or diabodies rather than whole antibodies. Diabodies and scFv can be constructed without an Fc region, using only variable domains, potentially reducing the effects of anti-idiotypic reaction. Other forms of bispecific antibodies include the single chain “Janusins”. Bispecific diabodies, as opposed to bispecific whole antibodies, may also be useful because they can be readily constructed and expressed in E. coli. Diabodies (and many other polypeptides such as antibody fragments) of appropriate binding specificities can be readily selected using phage display from libraries. If one arm of the diabody is to be kept constant, for instance, with a specificity directed against an antigen of interest, then a library can be made where the other arm is varied, and an antibody of appropriate specificity selected. An “antigen binding domain” is the part of an antibody which comprises the area which specifically binds to and is complementary to part or all of an antigen. Where an antigen is large, an antibody may only bind to a particular part of the antigen, which part is termed an epitope. An antigen binding domain may be provided by one or more antibody variable domains. An antigen binding domain may comprise an antibody light chain variable region (V_{L}) and an antibody heavy chain variable region (V_{H}).

In some embodiments, the molecular cargo may comprise a transgene encoding an antibody-like molecule that has been designed to specifically bind a target molecule and/or cell of the disclosure. In some embodiments the molecular cargo or transgene encoding the molecular cargo may comprise a TCR-mimic antibody. In some embodiments, such TCR-mimic antibodies can comprise high-affinity soluble antibody molecules endowed with a TCR-like specificity towards tumor or viral epitopes that can target tumor and/or virus-infected cells and mediate their specific killing.

Also encompassed within the present disclosure are transgenes encoding binding moieties based on engineered protein scaffolds or “alternative scaffolds”. Alternative scaffolds are derived from stable, soluble, natural protein structures which have been modified to provide a binding site for a target molecule of interest. Examples of alternative scaffolds include, but are not limited to, affibodies, which are based on the Z-domain of staphylococcal protein A that provides a binding interface on two of its α-helices; anticalins, derived from lipocalins, that incorporate binding sites for small ligands at the open end of a β-barrel fold; monobodies, designed to incorporate the fibronectin type III domain (Fn3) of fibronectin or tenascin as a protein scaffold or synthetic FN3 domains (e.g., tencon); nanobodies, and DARPins. Additional alternative scaffolds include Adnectin™, iMab, EETI-II/AGRP, Kunitz domain, thioredoxin peptide aptamer, Affilin, Tetranectin, Fynomer, and Avimer. Alternative scaffolds are typically targeted to bind the same antigenic proteins as antibodies and are potential therapeutic agents. Alternative scaffolds may, for example, act as inhibitors or antagonists, or as delivery vehicles to target molecules, such as toxins, to a specific tissue in vivo. Short peptides may also be used to bind a target protein. Phylomers are natural structured peptides derived from bacterial genomes. Such peptides represent a diverse array of protein structural folds and can be used to inhibit/disrupt protein-protein interactions in vivo.

Alternative scaffolds are typically single chain polypeptidic frameworks that contain a highly structured core associated with variable domains of high conformational tolerance allowing insertions, deletions, or other substitutions within the variable domains. Libraries introducing diversity to one or more variable domains, and in some cases to the structured core, may be generated using known protocols and the resulting libraries may be screened for binding to a target molecule and/or cell of the disclosure, and the identified binders may be further characterized for their specificity using known methods. Alternative scaffolds may be derived from Protein A, in particular, the Z-domain thereof (affibodies), ImmE7 (immunity proteins), BPTI/APPI (Kunitz domains), CTLA-4, charybdotoxin (Scorpion toxin), Min-23 (knottins), lipocalins (anticalins), Ras-binding protein AF-6 (PDZ-domains), neokarzinostatin, a fibronectin domain, an ankyrin consensus repeat domain, or thioredoxin.

In some embodiments, the molecular cargo comprises a transgene encoding a TCR. TCRs are described using the International Immunogenetics (IMGT) TCR nomenclature, and the IMGT public database of TCR sequences.

The TCRs of the present disclosure may be in any format. For example, the TCRs may be αβ heterodimers, or αα or ββ homodimers. α/β heterodimeric TCRs have an α-chain and a β-chain. Broadly, each chain comprises variable, joining and constant region, and the β-chain also usually contains a short diversity region between the variable and joining regions, but this diversity region is often considered as part of the joining region. Each variable region comprises three hypervariable CDRs (Complementarity Determining Regions) embedded in a framework sequence; CDR3 is believed to be the main mediator of antigen recognition. There are several types of α-chain variable (Vα) regions and several types of R-chain variable (Vβ) regions distinguished by their framework, CDR1 and CDR2 sequences, and by a partly defined CDR3 sequence.

The TCRs of the disclosure may not correspond to TCRs as they exist in nature. For example, they may comprise α- and β-chain combinations that are not present in a natural repertoire. Alternatively, or additionally, a TCR described herein may be soluble, and/or the α- and/or β-chain constant domain may be truncated relative to the native/naturally occurring TRAC/TRBC sequences such that, for example, the C-terminal transmembrane domain and intracellular regions are not present. Such truncation may result in removal of the cysteine residues from TRAC/TRBC that form the native interchain disulfide bond.

In addition, the TRAC/TRBC domains may contain modifications. For example, the α-chain extracellular sequence may include a modification relative to the native/naturally occurring TRAC whereby amino acid T48 of TRAC, with reference to IMGT numbering, is replaced with C48. Likewise, the β-chain extracellular sequence may include a modification relative to the native/naturally occurring TRBC1 or TRBC2 whereby S57 of TRBC1 or TRBC2, with reference to IMGT numbering, is replaced with C57. These cysteine substitutions relative to the native α- and β-chain extracellular sequences enable the formation of a non-native interchain disulfide bond which stabilizes the refolded soluble TCR, i.e., the TCR formed by refolding extracellular α- and β-chains. This non-native disulfide bond facilitates the display of correctly folded TCRs on phage. In addition, the use of the stable disulfide linked soluble TCR enables more convenient assessment of binding affinity and binding half-life. Alternative positions for the formation of a non-native disulfide include, for example, Thr 45 of exon 1 of TRAC*01 and Ser 77 of exon 1 of TRBC1*01 or TRBC2*01; Tyr 10 of exon 1 of TRAC*01 and Ser 17 of exon 1 of TRBC1*01 or TRBC2*01; Thr 45 of exon 1 of TRAC*01 and Asp 59 of exon 1 of TRBC1*01 or TRBC2*01; and Ser 15 of exon 1 of TRAC*01 and Glu 15 of exon 1 of TRBC1*01 or TRBC2*01. TCRs with a non-native disulfide bond may be full length or may be truncated.

TCRs of the disclosure may be in single chain format. Single chain TCRs include a TCR polypeptides of the type: Vα-L-Vβ, Vβ-L-Vα, Vα-Cα-L-Vβ, Vα-L-VP-Cβ or Vα-Cα-L-Vβ-Cβ, optionally in the reverse orientation, wherein Vα and Vβ are TCR α and β variable regions respectively, Ca and CP are TCR α and β constant regions respectively, and L is a linker sequence. Single chain TCRs may contain a non-native disulfide bond. The TCR may be in a soluble form (i.e., having no transmembrane or cytoplasmic domains) or may contain full length α- and β-chains. The TCR may be provided on the surface of a cell, such as a T cell.

TCRs of the disclosure may be engineered to include mutations. Methods for producing mutated high affinity TCR variants such as phage display and site directed mutagenesis. Preferably, mutations to improve affinity are made within the variable regions of α- and/or β-chains. More preferably mutations to improve affinity are made within the CDRs. There may be between 1 and 15 mutations in the α- and or β-chain variable regions.

TCRs of the disclosure may also be labeled with an imaging compound, for example a label that is suitable for diagnostic purposes. Such labelled high affinity TCRs are useful in a method for detecting a TCR ligand selected from CD1-antigen complexes, bacterial superantigens, and MHC-peptide/superantigen complexes, which method comprises contacting the TCR ligand with a high affinity TCR (or a multimeric high affinity TCR complex) which is specific for the TCR ligand; and detecting binding to the TCR ligand. In multimeric high affinity TCR complexes (formed, for example, using biotinylated heterodimers) fluorescent streptavidin can be used to provide a detectable label. A fluorescently-labelled multimer is suitable for use in FACS analysis, for example to detect antigen presenting cells carrying the peptide for which the high affinity TCR is specific.

A TCR of the present disclosure (or multivalent complex thereof) may alternatively or additionally be associated with (e.g., fused to) a therapeutic agent which may be, for example, a toxic moiety for use in cell killing, or an immunostimulating agent such as an interleukin or a cytokine. A multivalent high affinity TCR complex of the present disclosure may have enhanced binding capability for a TCR ligand compared to a non-multimeric wild-type or high affinity T cell receptor heterodimer. Thus, the multivalent high affinity TCR complexes according to the disclosure are particularly useful for tracking or targeting cells presenting particular antigens in vitro or in vivo and are also useful as intermediates for the production of further multivalent high affinity TCR complexes having such uses. The high affinity TCR or multivalent high affinity TCR complex may therefore be provided in a pharmaceutically acceptable formulation for use in vivo.

The TCR may be encoded either in a single open reading frame or two distinct open reading frames. Also included in the scope of the disclosure is a cell harboring a first expression vector which comprises nucleic acid encoding an α-chain of a TCR of the disclosure, and a second expression vector which comprises nucleic acid encoding a β-chain of a TCR of the disclosure. Alternatively, one vector may encode both an α- and a β-chain of a TCR of the disclosure.

The TCRs of the disclosure intended for use in adoptive therapy are generally glycosylated when expressed by the transfected T cells. The glycosylation pattern of transfected TCRs may be modified by mutations of the transfected gene.

In some embodiments, the molecular cargo described herein may comprise a transgene encoding a chimeric antigen receptor (CAR). CARs are genetically engineered receptors. CARs may be generated that bind a target molecule and/or cell of the present disclosure by incorporating an antigen binding domain that specifically binds the target molecular and/or cell to the extracellular domain of the CAR. CARs may be introduced into and expressed by immune cells, such as T cells, NK cells, or macrophages. CARs can be programmed to both recognize a specific antigen and, when bound to that antigen, activate the immune cell to attack and destroy the cell presenting that antigen. When these antigens exist on tumor cells, an immune cell that expresses the CAR can target and kill the tumor cell.

The general structure of a CAR typically comprises an extracellular domain that binds the antigen, a hinge, a transmembrane domain, and an intercellular domain comprising a signaling domain and optionally one or more co-stimulatory domains.

Extracellular domains of the CAR may contain any polypeptide that specifically binds the desired antigen. For example, the extracellular domain may comprise an antibody fragment such as scFv or V_{HH}. The CARs may also be engineered to bind two or more desired antigens that may be arranged in tandem and separated by linker sequences. For example, one or more domain antibodies, scFvs, llama VHH antibodies or other V_{H} only antibody fragments may be organized in tandem via a linker to provide bispecificity or multispecificity to the CAR.

A hinge domain may be present between the extracellular domain and the transmembrane domain of the CAR, e.g., to provide flexibility to allow effective binding of the extracellular domain to its intended target. The hinge domain may be a polypeptide of about 2 to 100 amino acids in length. The hinge may include or be composed of flexible residues such as Gly and Ser so that the adjacent protein domains are free to move relative to one another. Longer hinges may be used when it is desirable to ensure that two adjacent domains do not sterically interfere with one another. The hinge may be derived from a hinge region or portion of the hinge region of any immunoglobulin. Non-limiting examples of linkers include a part of human CD8α chain, extracellular domain of CD28, an Ig hinge from IgG, IgM, IgA, IgD, or IgE, FcγRllla receptor, or a functional fragment thereof.

Transmembrane domains of the CAR may be derived transmembrane proteins, such as an alpha, beta or zeta chain of a T-cell receptor, CD28, CD3 epsilon, CD2, CD4, CD5, CD8, CD9, CD16, CD18, CD19, CD22, CD27, CD29, CD33, CD37, CD40, CD45, CD49a, CD64, CD80, CD84, CD86, CD96 (Tactile), CD100 (SEMA4D), CD103, CD134, CD154, CD160 (BY55), KIRDS2, OX40, LFA-1 (CD11a, CD18), CD11b, CD11c, CD11d, ICOS (CD278), 4-1 BB (CD137), 4-1 BBL, GITR, BAFFR, HVEM (LIGHTR), SLAMF7, NKp80 (KLRFI), IL2R beta, IL2R gamma, IL7Ra, ITGA1, VLA1, ITGA4, IA4, CD49D, ITGA6, VLA-6, CD49f, ITGAD, ITGAE, ITGAL, LFA-1, ITGAM, ITGAX, ITGB1, ITGB2, LFA-1, ITGB7, TNFR2, DNAM1 (CD226), SLAMF4 (CD244, 2B4), CEACAMI, CRT AM, Ly9 (CD229), PSGL1, SLAMF6 (NTB-A, Ly108), SLAM (SLAMF1, CD150, IPO-3), BLAME (SLAMF8), SELPLG (CD162), LTBR, PAG/Cbp, NKp30, NKp44, NKp46, NKG2D, and NKG2C, or functional fragment thereof.

The intracellular signaling domain of a CAR participates in transducing the signal of effective CAR binding to a target antigen into the interior of the immune effector cell to elicit an effector cell function, e.g., activation, cytokine production, proliferation, and cytotoxic activity, including the release of cytotoxic factors to the CAR-bound target cell, or other cellular responses elicited following antigen binding to the extracellular CAR domain. Non-limiting examples of intracellular signaling domains of the CAR include those derived from CD3ζ, CD3ε, CD3δ, CD3γ, CD5, CD22, CD39, CD79A, CD79B, CD66d, CD226, DAP10, DAP12, Fc epsilon receptor I gamma chain (FCER1G), or FcR β.

Intracellular co-stimulatory domains of the CAR can provide a second signal required for efficient activation and function of T lymphocytes upon binding to antigen. Such co-stimulatory domains may be derived from one or more co-stimulatory molecules, such as, but not limited to, 4-1BB, CD2, CD7, CD27, CD28, CD30, CD40, CD54 (ICAM), CD83, CD134 (OX40), CD150 (SLAMF1), CD152 (CTLA4), CD223 (LAG3), CD270 (HVEM), CD278 (ICOS), DAP10, LAT, NKD2C SLP76, TRIM, BTLA, GITR, CD226, HVEM, and ZAP70.

The CARs can be generated by standard molecular biology techniques. The extracellular domain that binds the desired antigen may be derived from the antibodies or their antigen binding fragments described herein.

The transgene encoding a molecular cargo described herein may comprise any number of classes of additional of genes. Non-limiting examples of classes of additional genes include (a) a second targeting moiety, such as antibodies, including fragments thereof and bispecific antibodies (e.g., bispecific T cell engagers (BiTEs)), (b) secretable cytokines (e.g., GM-CSF, IL-7, IL-12, IL-15, IL-18), (c) membrane bound cytokines (e.g., IL-15), (d) chimeric cytokine receptors (e.g., IL-2/IL-7, IL-4/IL-7), (e) constitutive active cytokine receptors (e.g., C7R), (f) dominant negative receptors (DNR; e.g., TGFRII DNR), (g) ligands of co-stimulatory molecules (e.g., CD80, 4-1BBL), (h) nuclear factor of activated T cells (NFATs) (e.g., NFATc1, NFATc2, NFATc3, NFATc4, and NFAT5), or (j) suicide genes (e.g., CD20, truncated EGFR or HER2, inducible caspase 9 molecules).

In some embodiments, the molecular cargo described herein may comprise a transgene encoding an accessory molecule which can, for example, modulate survival or an activity of a target cell.

Non-limiting examples of useful accessory molecules include, e.g., an anti-CD28 antibody, an anti-CD80 (B7.1) antibody, an anti-CD86 (B7.2) antibody, an anti-anti-CD3 antibody, an anti-CD2 antibody, an anti-CD4 antibody, an anti-CD8 antibody, an anti-CD47 antibody, and functional derivatives, mutants and fragments thereof.

Accessory molecules include molecules that can provide a signal which, mediates a cell response, including, but not limited to, proliferation, activation, differentiation, and the like.

The accessory molecule can be, for example, an inhibitory or stimulatory antibody, a peptide ligand, a costimulatory peptide, a cytokine, etc. Non-limiting examples of accessory molecules include, e.g., CD7, B7.1 (CD80), B7.2 (CD86), PD-L1, PD-L2, 4-1BBL, OX40L, Fas ligand (FasL), inducible co stimulatory ligand (ICOS-L), intercellular adhesion molecule (ICAM), CD30L, CD40, CD70, CD83, HLA-G, MICA, MICB, FIVEM, lymphotoxin β receptor, 3/TR6, ILT3, ILT4, HVEM, an agonist or antibody that binds Toll ligand receptor and a ligand that specifically binds to B7-H3 as well as antibodies that specifically bind to CD27, CD28, B7.1 (CD80), B7.2 (CD86), 4-1BB, OX40, CD30, CD40, PD-1, ICOS, lymphocyte function-associated antigen-1 (LFA-1), CD2, CD3, CD7, LIGHT, NKG2C, B7-H3, and a ligand that specifically binds to CD83.

Additional non-limiting examples of accessory molecules include, e.g., TNF/TNF family members (e.g., OX40L, ICOSL, FASL, LTA, LTB TRAIL, CD153, TNFSF9, RANKL, TWEAK, TNFSF13, TNFSF13b, TNFSF14, TNFSF15, TNFSF18, CD40LG, CD70); members of the Immunoglobulin superfamily (e.g., VISTA, PD1, PD-L1, PD-L2, B71, B72, CTLA4, CD28, TIM3, CD4, CD8, CD19, T cell receptor chains, ICOS, ICOS ligand, HHLA2, butyrophilms, BTLA, B7-H3, B7-H4, CD3, CD79a, CD79b, IgSF CAMS (including CD2, CD58, CD48, CD150, CD229, CD244, ICAM-1), Leukocyte immunoglobulin like receptors (LILR), killer cell immunoglobulin like receptors (KIR)), lectin superfamily members, selectins, cytokines/chemokine and cytokine/chemokine receptors, growth factors and growth factor receptors), adhesion molecules (integrins, fibronectins, cadherins), or ecto-domains of multi-span integral membrane proteins, or antibodies directed to any of these molecules.

In some embodiments, a molecular cargo described herein can encode a toxic protein or peptide, e.g., a cytotoxic agent. In one specific embodiment, the cytotoxic agent can be a toxin or a radioactive isotope (e.g., a radioconjugate) or a suicide gene. Non-limiting examples of toxins include, e.g., enzymatically active toxins of bacterial, fungal, plant, or animal origin, or fragments, mutants or derivatives thereof. Enzymatically active toxins and fragments thereof that can be used include, for example, diphtheria A chain, nonbinding active fragments of diphtheria toxin, exotoxin A chain (from Pseudomonas aeruginosa), ricin A chain, abrin A chain, modeccin A chain, α-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. Non-limiting examples of suicide genes include, e.g., thymidine kinase, cytosine deaminase, purine nucleoside phosphorylase, nitroreductase, β-galactosidase, hepatic cytochrome P450-2B1, linamarase, horseradish peroxidase, and carboxypeptidase.

In various embodiments, a molecular cargo of the present disclosure can include a gene editing system or components of such systems. Various known gene editing systems can be used in the practice of the present disclosure, including, e.g., a Clustered Regularly Interspersed Short Palindromic Repeats (CRISPR)/Cas system; zinc finger nuclease (ZFN) system; transcription activator-like effector nuclease (TALEN) system, or systems using meganucleases, restriction endonucleases, or recombinases. Generally, these gene editing systems are used to modify a genome within a cell by inducing a double strand break (DSB) or a nick (e.g., a single strand break, or SSB) in a target DNA sequence. Cleavage or nicking can occur through the use of specific nucleases such as engineered ZFN, TALENs, or using the CRISPR/Cas system with an engineered guide RNA (gRNA) to guide specific cleavage or nicking of a target DNA sequence. Further, targeted nucleases have been developed, and additional nucleases are being developed, for example based on the Argonaute system (e.g., from T. thermophilus, known as ‘TtAgo’, see Swarts et al (2014) Nature 507(7491): 258-261), which also may have the potential for uses in genome editing and gene therapy.

Molecular cargoes described herein may comprise a polynucleotide molecule such as but not limited to guide nucleic acids (e.g., Cas9 guide RNAs), interfering nucleic acids (e.g., shRNAs, siRNAs, microRNAs, antisense oligonucleotides, gapmers), mixmers, ribozymes, phosphorodiamidite morpholinos, peptide nucleic acids, aptamers, and mRNAs, etc. In various embodiments, such polynucleotide molecules may comprise one or more modified nucleotides. In various embodiments, a polynucleotide molecule described herein may comprise one or more modified inter-nucleotide linkage. Polynucleotides may be single-stranded or double-stranded.

In some embodiments, the molecular cargo comprises at least one polynucleotide molecule. In some embodiments, the molecular cargo comprises at least 2, at least 3, at least 4, at least 5, or at least 10 polynucleotide molecules.

In some embodiments, the polynucleotide molecule is DNA. In some embodiments, the polynucleotide molecule is RNA.

In various embodiments, a polynucleotide described herein (e.g., interfering nucleic acid or guide RNA) may comprise a region of complementarity to a target nucleic acid which can be in the range of 8 to 15, 8 to 30, 8 to 40, or 10 to 50, or 5 to 50, or 5 to 40 nucleotides in length. In certain embodiments, a region of complementarity of a polynucleotide to a target nucleic acid may be 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, the region of complementarity may be complementary with at least 10 consecutive nucleotides of a target nucleic acid. In some embodiments, a polynucleotide may contain 1, 2, 3, 4 or 5 base mismatches compared to the portion of the consecutive nucleotides of target nucleic acid. In some embodiments the polynucleotide may have up to 3 mismatches over 15 bases, or up to 4 mismatches over 10 bases. In some embodiments, the polynucleotide is complementary (e.g., at least 80%, at least 85% at least 90%, at least 95%, or 100%) to a target sequence of any one of the polynucleotides of the present disclosure. In various embodiments, such target sequence may be 100% complementary to the polynucleotide described herein. In some embodiments, any one or more of the thymine bases (T's) in any one of the polynucleotides described herein may be uracil bases (U's), and/or any one or more of the U's may be T's. A target sequence described herein may comprise a sequence of nucleic acid in a target gene that has complementarity to the guide sequence of the gRNA. The interaction of the target sequence and the guide sequence directs an RNA-guided DNA-binding agent (e.g., Cas protein) to bind, and potentially nick or cleave (depending on the activity of the agent), within the target sequence.

The polynucleotides described herein may be modified, e.g., comprise a modified nucleotide, a modified internucleoside linkage, and/or a modified sugar moiety, or combinations thereof. In addition, polynucleotides can possess one or more of the following properties: have improved cell uptake compared to unmodified polynucleotides; are not toxic to cells or mammals are not immune stimulatory; avoid pattern recognition receptors do not mediate alternative splicing; are nuclease resistant; have improved endosomal exit internally in a cell; or minimizes TLR stimulation. Any of the various modified chemistries or formats of polynucleotides disclosed herein may be combined with together. As a non-limiting example, one, two, three, four, five, six, seven, eight or more different types of modifications may be included within the same polynucleotide.

In various embodiments, particular nucleotide modification(s) may be used that render a polynucleotide into which the modification(s) are incorporated more resistant to nuclease digestion than the native oligoribonucleotide or oligodeoxynucleotide molecules; such modified polynucleotides survive intact for a longer time than unmodified polynucleotides. Exemplary modified polynucleotides include those comprising modified backbones, for example, modified internucleoside linkages such as, methyl phosphonates, phosphotriesters, phosphorothioates short chain alkyl or cycloalkyl intersugar linkages heterocyclic intersugar linkages or short chain heteroatomic or. As such, polynucleotides described herein may be stabilized against nucleolytic degradation, e.g., via incorporation of a modification, e.g., a nucleotide modification.

In various embodiments, a polynucleotide may be of up to 50 nucleotides in length in which 2 to 10, 2 to 15, 2 to 16, 2 to 17, 2 to 18, 2 to 19, 2 to 20, 2 to 25, 2 to 30, 2 to 40, or 2 to 45, nucleotides of the polynucleotide may be modified nucleotides. The polynucleotide may be of 8 to 30 nucleotides in length in which 2 to 10, 2 to 15, 2 to 16, 2 to 17, 2 to 18, 2 to 19, 2 to 20, 2 to 25, 2 to 30 nucleotides of the polynucleotide can be modified nucleotides. In some embodiments, the polynucleotide may be of 8 to 15 nucleotides in length in which 2 to 4, 2 to 5, 2 to 6, 2 to 7, 2 to 8, 2 to 9, 2 to 10, 2 to 11, 2 to 12, 2 to 13, 2 to 14 nucleotides of the polynucleotide are modified nucleotides. In some embodiments, the polynucleotides can have every nucleotide except 1, 2, 3, 4, 5, 6, 7, 8, 9, 10, 11, 12, 13, 14, or 15 nucleotides modified.

In some embodiments, a molecular cargo comprises a guide RNA or a DNA encoding a guide RNA. A “guide RNA” or “gRNA” is an RNA molecule that binds to a Cas protein (e.g., Cas9 protein) and targets the Cas protein to a specific location within a target DNA. Guide RNAs can comprise two segments: a “DNA-targeting segment” (also called “guide sequence”) and a “protein-binding segment.” “Segment” includes a section or region of a molecule, such as a contiguous stretch of nucleotides in an RNA. Some gRNAs, such as those for Cas9, can comprise two separate RNA molecules: an “activator-RNA” (e.g., tracrRNA) and a “targeter-RNA” (e.g., CRISPR RNA or crRNA). Other gRNAs are a single RNA molecule (single RNA polynucleotide), which can also be called a “single-molecule gRNA,” a “single-guide RNA,” or an “sgRNA.” See, e.g., WO 2013/176772, WO 2014/065596, WO 2014/089290, WO 2014/093622, WO 2014/099750, WO 2013/142578, and WO 2014/131833, each of which is herein incorporated by reference in its entirety for all purposes. A guide RNA can refer to either a CRISPR RNA (crRNA) or the combination of a crRNA and a trans-activating CRISPR RNA (tracrRNA). The crRNA and tracrRNA can be associated as a single RNA molecule (single guide RNA or sgRNA) or in two separate RNA molecules (dual guide RNA or dgRNA). For Cas9, for example, a single-guide RNA can comprise a crRNA fused to a tracrRNA (e.g., via a linker). For Cpf1 and CasD, for example, only a crRNA is needed to achieve binding to a target sequence. The terms “guide RNA” and “gRNA” include both double-molecule (i.e., modular) gRNAs and single-molecule gRNAs. In some of the methods and compositions disclosed herein, a gRNA is a S. pyogenes Cas9 gRNA or an equivalent thereof. In some of the methods and compositions disclosed herein, a gRNA is a S. aureus Cas9 gRNA or an equivalent thereof.

The target DNA includes both the sequence on the complementary strand to which the guide RNA hybridizes and the corresponding sequence on the non-complementary strand (e.g., adjacent to the protospacer adjacent motif (PAM)). The term “guide RNA target sequence” as used herein refers specifically to the sequence on the non-complementary strand corresponding to (i.e., the reverse complement of) the sequence to which the guide RNA hybridizes on the complementary strand. That is, the guide RNA target sequence refers to the sequence on the non-complementary strand adjacent to the PAM (e.g., upstream or 5′ of the PAM in the case of Cas9). A guide RNA target sequence is equivalent to the DNA-targeting segment of a guide RNA, but with thymines instead of uracils. As one example, a guide RNA target sequence for an SpCas9 enzyme can refer to the sequence upstream of the 5′-NGG-3′ PAM on the non-complementary strand. A guide RNA is designed to have complementarity to the complementary strand of a target DNA, where hybridization between the DNA-targeting segment of the guide RNA and the complementary strand of the target DNA promotes the formation of a CRISPR complex. Full complementarity is not necessarily required, provided that there is sufficient complementarity to cause hybridization and promote formation of a CRISPR complex. If a guide RNA is referred to herein as targeting a guide RNA target sequence, what is meant is that the guide RNA hybridizes to the complementary strand sequence of the target DNA that is the reverse complement of the guide RNA target sequence on the non-complementary strand.

A target DNA or guide RNA target sequence can comprise any polynucleotide, and can be located, for example, in the nucleus or cytoplasm of a cell or within an organelle of a cell, such as a mitochondrion or chloroplast. A target DNA or guide RNA target sequence can be any nucleic acid sequence endogenous or exogenous to a cell. The guide RNA target sequence can be a sequence coding a gene product (e.g., a protein) or a non-coding sequence (e.g., a regulatory sequence) or can include both.

The target sequence (e.g., guide RNA target sequence) for the DNA-binding protein can be anywhere within a targeted gene that is suitable for altering expression of the targeted gene. As one example, the target sequence can be within a regulatory element, such as an enhancer or promoter, or can be in proximity to a regulatory element. For example, the target sequence can be within about 10, 20, 30, 40, 50, 100, 200, 300, 400, 500, or 1,000 nucleotides of the start codon.

Site-specific binding and cleavage of a target DNA by a Cas protein can occur at locations determined by both (i) base-pairing complementarity between the guide RNA and the complementary strand of the target DNA and (ii) a short motif, called the protospacer adjacent motif (PAM), in the non-complementary strand of the target DNA. The PAM can flank the guide RNA target sequence. Optionally, the guide RNA target sequence can be flanked on the 3′ end by the PAM (e.g., for Cas9). Alternatively, the guide RNA target sequence can be flanked on the 5′ end by the PAM (e.g., for Cpf1). For example, the cleavage site of Cas proteins can be about 1 to about 10 or about 2 to about 5 base pairs (e.g, 3 base pairs) upstream or downstream of the PAM sequence (e.g., within the guide RNA target sequence). In the case of SpCas9, the PAM sequence (i.e., on the non-complementary strand) can be 5′-NiGG-3′, where Ni is any DNA nucleotide, and where the PAM is immediately 3′ of the guide RNA target sequence on the non-complementary strand of the target DNA. As such, the sequence corresponding to the PAM on the complementary strand (i.e., the reverse complement) would be 5′-CCN2-3′, where N2 is any DNA nucleotide and is immediately 5′ of the sequence to which the DNA-targeting segment of the guide RNA hybridizes on the complementary strand of the target DNA. In some such cases, Ni and N2 can be complementary and the Ni—N2 base pair can be any base pair (e.g, Ni═C and N2=G; Ni=G and N2=C; Ni=A and N2=T, or Ni=T, and N2=A). In the case of Cas9 from S. aureus, the PAM can be NNGRRT (SEQ ID NO: 145) or NNGRR (SEQ ID NO: 146), where N can A, G, C, or T, and R can be G or A. In the case of Cas9 from C. jejuni, the PAM can be, for example, NNNNACAC (SEQ ID NO: 147) or NNNNRYAC (SEQ ID NO: 148), where N can be A, G, C, or T, and R can be G or A. In some cases (e.g., for FnCpf1), the PAM sequence can be upstream of the 5′ end and have the sequence 5′-TTN-3. In the case of DpbCasX, the PAM can have the sequence 5′-TTCN-3′. In the case of CasΦ, the PAM can have the sequence 5′-TBN-3′, wherein B is G, T, or C.

An example of a guide RNA target sequence is a 20-nucleotide DNA sequence immediately preceding an NGG motif recognized by an SpCas9 protein. The guanine at the 5′ end can facilitate transcription by RNA polymerase in cells. Other examples of guide RNA target sequences plus PAMs can include two guanine nucleotides at the 5′ end to facilitate efficient transcription by T7 polymerase in vitro. Other guide RNA target sequences plus PAMs can have between 4-22 nucleotides in length, including the 5′ G or GG and the 3′ GG or NGG. Yet other guide RNA target sequences plus PAMs can have between 14 and 20 nucleotides in length.

Formation of a CRISPR complex hybridized to a target DNA can result in cleavage of one or both strands of the target DNA within or near the region corresponding to the guide RNA target sequence (i.e., the guide RNA target sequence on the non-complementary strand of the target DNA and the reverse complement on the complementary strand to which the guide RNA hybridizes). For example, the cleavage site can be within the guide RNA target sequence (e.g., at a defined location relative to the PAM sequence). The “cleavage site” includes the position of a target DNA at which a Cas protein produces a single-strand break or a double-strand break. The cleavage site can be on only one strand (e.g., when a nickase is used) or on both strands of a double-stranded DNA. Cleavage sites can be at the same position on both strands (producing blunt ends; e.g., Cas9) or can be at different sites on each strand (producing staggered ends (i.e., overhangs); e.g., Cpf1). Staggered ends can be produced, for example, by using two Cas proteins, each of which produces a single-strand break at a different cleavage site on a different strand, thereby producing a double-strand break. For example, a first nickase can create a single-strand break on the first strand of double-stranded DNA (dsDNA), and a second nickase can create a single-strand break on the second strand of dsDNA such that overhanging sequences are created. In some cases, the guide RNA target sequence or cleavage site of the nickase on the first strand is separated from the guide RNA target sequence or cleavage site of the nickase on the second strand by at least 2, 3, 4, 5, 6, 7, 8, 9, 10, 15, 20, 25, 30, 40, 50, 75, 100, 250, 500, or 1,000 base pairs.

An exemplary two-molecule gRNA comprises a crRNA-like (“CRISPR RNA” or “targeter-RNA” or “crRNA” or “crRNA repeat”) molecule and a corresponding tracrRNA-like (“trans-activating CRISPR RNA” or “activator-RNA” or “tracrRNA”) molecule. A crRNA comprises both the DNA-targeting segment (single-stranded) of the gRNA and a stretch of nucleotides that forms one half of the dsRNA duplex of the protein-binding segment of the gRNA.

A corresponding tracrRNA (activator-RNA) comprises a stretch of nucleotides that forms the other half of the dsRNA duplex of the protein-binding segment of the gRNA. A stretch of nucleotides of a crRNA are complementary to and hybridize with a stretch of nucleotides of a tracrRNA to form the dsRNA duplex of the protein-binding domain of the gRNA. As such, each crRNA can be said to have a corresponding tracrRNA.

In systems in which both a crRNA and a tracrRNA are needed, the crRNA and the corresponding tracrRNA hybridize to form a gRNA. In systems in which only a crRNA is needed, the crRNA can be the gRNA. The crRNA additionally provides the single-stranded DNA-targeting segment that hybridizes to the complementary strand of a target DNA. If used for modification within a cell, the exact sequence of a given crRNA or tracrRNA molecule can be designed to be specific to the species in which the RNA molecules will be used.

The DNA-targeting segment (crRNA) of a given gRNA comprises a nucleotide sequence that is complementary to a sequence on the complementary strand of the target DNA, as described in more detail below. The DNA-targeting segment of a gRNA interacts with the target DNA in a sequence-specific manner via hybridization (i.e., base pairing). As such, the nucleotide sequence of the DNA-targeting segment may vary and determines the location within the target DNA with which the gRNA and the target DNA will interact. The DNA-targeting segment of a subject gRNA can be modified to hybridize to any desired sequence within a target DNA. Naturally occurring crRNAs differ depending on the CRISPR/Cas system and organism but often contain a targeting segment of between 21 to 72 nucleotides length, flanked by two direct repeats (DR) of a length of between 21 to 46 nucleotides (see, e.g., WO 2014/131833, herein incorporated by reference in its entirety for all purposes). In the case of S. pyogenes, the DRs are 36 nucleotides long and the targeting segment is 30 nucleotides long. The 3′ located DR is complementary to and hybridizes with the corresponding tracrRNA, which in turn binds to the Cas protein.

The DNA-targeting segment can have, for example, a length of at least about 12, at least about 15, at least about 17, at least about 18, at least about 19, at least about 20, at least about 25, at least about 30, at least about 35, or at least about 40 nucleotides. Such DNA-targeting segments can have, for example, a length from about 12 to about 100, from about 12 to about 80, from about 12 to about 50, from about 12 to about 40, from about 12 to about 30, from about 12 to about 25, or from about 12 to about 20 nucleotides. For example, the DNA targeting segment can be from about 15 to about 25 nucleotides (e.g., from about 17 to about 20 nucleotides, or about 17, 18, 19, or 20 nucleotides). For Cas9 from S. pyogenes, a typical DNA-targeting segment is between 16 and 20 nucleotides in length or between 17 and 20 nucleotides in length. For Cas9 from S. aureus, a typical DNA-targeting segment is between 21 and 23 nucleotides in length. For Cpf1, a typical DNA-targeting segment is at least 16 nucleotides in length or at least 18 nucleotides in length.

In one example, the DNA-targeting segment can be about 20 nucleotides in length. However, shorter and longer sequences can also be used for the targeting segment (e.g., 15-25 nucleotides in length, such as 15, 16, 17, 18, 19, 20, 21, 22, 23, 24, or 25 nucleotides in length). The degree of identity between the DNA-targeting segment and the corresponding guide RNA target sequence (or degree of complementarity between the DNA-targeting segment and the other strand of the guide RNA target sequence) can be, for example, about 75%, about 80%, about 85%, about 90%, about 95%, or 100%. The DNA-targeting segment and the corresponding guide RNA target sequence can contain one or more mismatches. For example, the DNA-targeting segment of the guide RNA and the corresponding guide RNA target sequence can contain 1-4, 1-3, 1-2, 1, 2, 3, or 4 mismatches (e.g., where the total length of the guide RNA target sequence is at least 17, at least 18, at least 19, or at least 20 or more nucleotides). For example, the DNA-targeting segment of the guide RNA and the corresponding guide RNA target sequence can contain 1-4, 1-3, 1-2, 1, 2, 3, or 4 mismatches where the total length of the guide RNA target sequence 20 nucleotides.

TracrRNAs can be in any form (e.g., full-length tracrRNAs or active partial tracrRNAs) and of varying lengths. They can include primary transcripts or processed forms. For example, tracrRNAs (as part of a single-guide RNA or as a separate molecule as part of a two-molecule gRNA) may comprise, consist essentially of, or consist of all or a portion of a wild type tracrRNA sequence (e.g., about or more than about 20, 26, 32, 45, 48, 54, 63, 67, 85, or more nucleotides of a wild type tracrRNA sequence). Examples of wild type tracrRNA sequences from S. pyogenes include 171-nucleotide, 89-nucleotide, 75-nucleotide, and 65-nucleotide versions. Examples of tracrRNAs within single-guide RNAs (sgRNAs) include the tracrRNA segments found within +48, +54, +67, and +85 versions of sgRNAs, where “+n” indicates that up to the +n nucleotide of wild type tracrRNA is included in the sgRNA.

The percent complementarity between the DNA-targeting segment of the guide RNA and the complementary strand of the target DNA can be at least 60% (e.g., at least 65%, at least 70%, at least 75%, at least 80%, at least 85%, at least 90%, at least 95%, or 100%). The percent complementarity between the DNA-targeting segment and the complementary strand of the target DNA can be at least 60% over about 20 contiguous nucleotides. As an example, the percent complementarity between the DNA-targeting segment and the complementary strand of the target DNA can be 100% over the 14 contiguous nucleotides at the 5′ end of the complementary strand of the target DNA and as low as 0% over the remainder. In such a case, the DNA-targeting segment can be considered to be 14 nucleotides in length. As another example, the percent complementarity between the DNA-targeting segment and the complementary strand of the target DNA can be 100% over the seven contiguous nucleotides at the 5′ end of the complementary strand of the target DNA and as low as 0% over the remainder. In such a case, the DNA-targeting segment can be considered to be 7 nucleotides in length. In some guide RNAs, at least 17 nucleotides within the DNA-targeting segment are complementary to the complementary strand of the target DNA. For example, the DNA-targeting segment can be 20 nucleotides in length and can comprise 1, 2, or 3 mismatches with the complementary strand of the target DNA. In one example, the mismatches are not adjacent to the region of the complementary strand corresponding to the protospacer adjacent motif (PAM) sequence (i.e., the reverse complement of the PAM sequence) (e.g., the mismatches are in the 5′ end of the DNA-targeting segment of the guide RNA, or the mismatches are at least 2, 3, 4, 5, 6, 7, 8, 9, 10, 11, 12, 13, 14, 15, 16, 17, 18, or 19 base pairs away from the region of the complementary strand corresponding to the PAM sequence).

The protein-binding segment of a gRNA can comprise two stretches of nucleotides that are complementary to one another. The complementary nucleotides of the protein-binding segment hybridize to form a double-stranded RNA duplex (dsRNA). The protein-binding segment of a subject gRNA interacts with a Cas protein, and the gRNA directs the bound Cas protein to a specific nucleotide sequence within target DNA via the DNA-targeting segment.

Single-guide RNAs can comprise a DNA-targeting segment and a scaffold sequence (i.e., the protein-binding or Cas-binding sequence of the guide RNA). Guide RNAs targeting any of the guide RNA target sequences disclosed herein can include, for example, a DNA-targeting segment on the 5′ end of the guide RNA fused to any of the exemplary guide RNA scaffold sequences on the 3′ end of the guide RNA. That is, any of the DNA-targeting segments disclosed herein can be joined to the 5′ end of any one of the above scaffold sequences to form a single guide RNA (chimeric guide RNA).

Guide RNAs can include modifications or sequences that provide for additional desirable features (e.g., modified or regulated stability; subcellular targeting; tracking with a fluorescent label; a binding site for a protein or protein complex; and the like). That is, guide RNAs can include one or more modified nucleosides or nucleotides, or one or more non-naturally and/or naturally occurring components or configurations that are used instead of or in addition to the canonical A, G, C, and U residues. Examples of such modifications include, for example, a 5′ cap (e.g., a 7-methylguanylate cap (m7G)); a 3′ polyadenylated tail (i.e., a 3′ poly(A) tail); a riboswitch sequence (e.g., to allow for regulated stability and/or regulated accessibility by proteins and/or protein complexes); a stability control sequence; a sequence that forms a dsRNA duplex (i.e., a hairpin); a modification or sequence that targets the RNA to a subcellular location (e.g., nucleus, mitochondria, chloroplasts, and the like); a modification or sequence that provides for tracking (e.g., direct conjugation to a fluorescent molecule, conjugation to a moiety that facilitates fluorescent detection, a sequence that allows for fluorescent detection, and so forth); a modification or sequence that provides a binding site for proteins (e.g., proteins that act on DNA, including transcriptional activators, transcriptional repressors, DNA methyltransferases, DNA demethylases, histone acetyltransferases, histone deacetylases, and the like); and combinations thereof. Other examples of modifications include engineered stem loop duplex structures, engineered bulge regions, engineered hairpins 3′ of the stem loop duplex structure, or any combination thereof. A bulge can be an unpaired region of nucleotides within the duplex made up of the crRNA-like region and the minimum tracrRNA-like region. A bulge can comprise, on one side of the duplex, an unpaired 5′-XXXY-3′ where X is any purine and Y can be a nucleotide that can form a wobble pair with a nucleotide on the opposite strand, and an unpaired nucleotide region on the other side of the duplex.

In some cases, a guide RNA for use in a transcriptional activation system comprising a dCas9-VP64 fusion protein paired with MS2-p65-HSF1 can be used. Guide RNAs in such systems can be designed with aptamer sequences appended to sgRNA tetraloop and stem-loop 2 designed to bind dimerized MS2 bacteriophage coat proteins.

Guide RNAs can comprise modified nucleosides and modified nucleotides including, for example, one or more of the following: (1) alteration or replacement of one or both of the non-linking phosphate oxygens and/or of one or more of the linking phosphate oxygens in the phosphodiester backbone linkage (an exemplary backbone modification); (2) alteration or replacement of a constituent of the ribose sugar such as alteration or replacement of the 2′ hydroxyl on the ribose sugar (an exemplary sugar modification); (3) replacement (e.g., wholesale replacement) of the phosphate moiety with dephospho linkers (an exemplary backbone modification); (4) modification or replacement of a naturally occurring nucleobase, including with a non-canonical nucleobase (an exemplary base modification); (5) replacement or modification of the ribose-phosphate backbone (an exemplary backbone modification); (6) modification of the 3′ end or 5′ end of the oligonucleotide (e.g., removal, modification or replacement of a terminal phosphate group or conjugation of a moiety, cap, or linker (such 3′ or 5′ cap modifications may comprise a sugar and/or backbone modification); and (7) modification or replacement of the sugar (an exemplary sugar modification). Other possible guide RNA modifications include modifications of or replacement of uracils or poly-uracil tracts. Similar modifications can be made to Cas-encoding nucleic acids, such as Cas mRNAs. For example, Cas mRNAs can be modified by depletion of uridine using synonymous codons.

Chemical modifications such as those listed above can be combined to provide modified gRNAs and/or mRNAs comprising residues (nucleosides and nucleotides) that can have two, three, four, or more modifications. For example, a modified residue can have a modified sugar and a modified nucleobase. In one example, every base of a gRNA is modified (e.g., all bases have a modified phosphate group, such as a phosphorothioate group). For example, all or substantially all of the phosphate groups of a gRNA can be replaced with phosphorothioate groups. Alternatively or additionally, a modified gRNA can comprise at least one modified residue at or near the 5′ end. Alternatively or additionally, a modified gRNA can comprise at least one modified residue at or near the 3′ end.

Some gRNAs comprise one, two, three or more modified residues. For example, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% of the positions in a modified gRNA can be modified nucleosides or nucleotides.

Unmodified nucleic acids can be prone to degradation. Exogenous nucleic acids can also induce an innate immune response. Modifications can help introduce stability and reduce immunogenicity. Some gRNAs described herein can contain one or more modified nucleosides or nucleotides to introduce stability toward intracellular or serum-based nucleases. Some modified gRNAs described herein can exhibit a reduced innate immune response when introduced into a population of cells.

The gRNAs disclosed herein can comprise a backbone modification in which the phosphate group of a modified residue can be modified by replacing one or more of the oxygens with a different substituent. The modification can include the wholesale replacement of an unmodified phosphate moiety with a modified phosphate group as described herein. Backbone modifications of the phosphate backbone can also include alterations that result in either an uncharged linker or a charged linker with unsymmetrical charge distribution.

Examples of modified phosphate groups include, phosphorothioate, phosphoroselenates, borano phosphates, borano phosphate esters, hydrogen phosphonates, phosphoroamidates, alkyl or aryl phosphonates and phosphotriesters. The phosphorous atom in an unmodified phosphate group is achiral. However, replacement of one of the non-bridging oxygens with one of the above atoms or groups of atoms can render the phosphorous atom chiral. The stereogenic phosphorous atom can possess either the “R” configuration (Rp) or the “S” configuration (Sp). The backbone can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens.

The phosphate group can be replaced by non-phosphorus containing connectors in certain backbone modifications. In some embodiments, the charged phosphate group can be replaced by a neutral moiety. Examples of moieties which can replace the phosphate group can include, without limitation, e.g., methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino.

Scaffolds that can mimic nucleic acids can also be constructed wherein the phosphate linker and ribose sugar are replaced by nuclease resistant nucleoside or nucleotide surrogates. Such modifications may comprise backbone and sugar modifications. In some embodiments, the nucleobases can be tethered by a surrogate backbone. Examples can include, without limitation, the morpholino, cyclobutyl, pyrrolidine and peptide nucleic acid (PNA) nucleoside surrogates.

The modified nucleosides and modified nucleotides can include one or more modifications to the sugar group (a sugar modification). For example, the 2′ hydroxyl group (OH) can be modified (e.g., replaced with a number of different oxy or deoxy substituents. Modifications to the 2′ hydroxyl group can enhance the stability of the nucleic acid since the hydroxyl can no longer be deprotonated to form a 2′-alkoxide ion.

Examples of 2′ hydroxyl group modifications can include alkoxy or aryloxy (OR, wherein “R” can be, e.g., alkyl, cycloalkyl, aryl, aralkyl, heteroaryl or a sugar); polyethyleneglycols (PEG), O(CH₂CH₂O)ₙCH₂CH₂OR wherein R can be, e.g., H or optionally substituted alkyl, and n can be an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20). The 2′ hydroxyl group modification can be 2′-O-Me. Likewise, the 2′ hydroxyl group modification can be a 2′-fluoro modification, which replaces the 2′ hydroxyl group with a fluoride. The 2′ hydroxyl group modification can include locked nucleic acids (LNA) in which the 2′ hydroxyl can be connected, e.g., by a C1-6 alkylene or C1-6 heteroalkylene bridge, to the 4′ carbon of the same ribose sugar, where exemplary bridges can include methylene, propylene, ether, or amino bridges; O-amino (wherein amino can be, e.g., NH2; alkylamino, dialkylamino, heterocyclyl, arylamino, diarylamino, heteroarylamino, or diheteroarylamino, ethylenediamine, or polyamino) and aminoalkoxy, O(CH₂)ₙ-amino, (wherein amino can be, e.g., NH₂; alkylamino, dialkylamino, heterocyclyl, arylamino, diarylamino, heteroarylamino, or diheteroarylamino, ethylenediamine, or polyamino). The 2′ hydroxyl group modification can include unlocked nucleic acids (UNA) in which the ribose ring lacks the C2′—C3′ bond. The 2′ hydroxyl group modification can include the methoxyethyl group (MOE), (OCH₂CH₂OCH₃, e.g., a PEG derivative).

Deoxy 2′ modifications can include hydrogen (i.e. deoxyribose sugars, e.g., at the overhang portions of partially dsRNA); halo (e.g., bromo, chloro, fluoro, or iodo); amino (wherein amino can be, e.g., NH2; alkylamino, dialkylamino, heterocyclyl, arylamino, diarylamino, heteroarylamino, diheteroarylamino, or amino acid); NH(CH₂CH₂NH)nCH₂CH₂— amino (wherein amino can be, e.g., as described herein), —NHC(O)R (wherein R can be, e.g., alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino as described herein.

The sugar modification can comprise a sugar group which may also contain one or more carbons that possess the opposite stereochemical configuration than that of the corresponding carbon in ribose. Thus, a modified nucleic acid can include nucleotides containing e.g., arabinose, as the sugar. The modified nucleic acids can also include abasic sugars. These abasic sugars can also be further modified at one or more of the constituent sugar atoms. The modified nucleic acids can also include one or more sugars that are in the L form (e.g., L-nucleosides).

The modified nucleosides and modified nucleotides described herein, which can be incorporated into a modified nucleic acid, can include a modified base, also called a nucleobase. Examples of nucleobases include, but are not limited to, adenine (A), guanine (G), cytosine (C), and uracil (U). These nucleobases can be modified or wholly replaced to provide modified residues that can be incorporated into modified nucleic acids. The nucleobase of the nucleotide can be independently selected from a purine, a pyrimidine, a purine analog, or pyrimidine analog. In some embodiments, the nucleobase can include, for example, naturally occurring and synthetic derivatives of a base.

In a dual guide RNA, each of the crRNA and the tracrRNA can contain modifications. Such modifications may be at one or both ends of the crRNA and/or tracrRNA. In a sgRNA, one or more residues at one or both ends of the sgRNA may be chemically modified, and/or internal nucleosides may be modified, and/or the entire sgRNA may be chemically modified. Some gRNAs comprise a 5′ end modification. Some gRNAs comprise a 3′ end modification.

As one example, nucleotides at the 5′ or 3′ end of a guide RNA can include phosphorothioate linkages (e.g., the bases can have a modified phosphate group that is a phosphorothioate group). For example, a guide RNA can include phosphorothioate linkages between the 2, 3, or 4 terminal nucleotides at the 5′ or 3′ end of the guide RNA. As another example, nucleotides at the 5′ and/or 3′ end of a guide RNA can have 2′-O-methyl modifications. For example, a guide RNA can include 2′-O-methyl modifications at the 2, 3, or 4 terminal nucleotides at the 5′ and/or 3′ end of the guide RNA (e.g., the 5′ end). Other possible modifications are described in more detail elsewhere herein. In a specific example, a guide RNA includes 2′-O-methyl analogs and 3′ phosphorothioate internucleotide linkages at the first three 5′ and 3′ terminal RNA residues. Such chemical modifications can, for example, provide greater stability and protection from exonucleases to guide RNAs, allowing them to persist within cells for longer than unmodified guide RNAs. Such chemical modifications can also, for example, protect against innate intracellular immune responses that can actively degrade RNA or trigger immune cascades that lead to cell death.

As one example, any of the guide RNAs described herein can comprise at least one modification. In one example, the at least one modification comprises a 2′-O-methyl (2′-O-Me) modified nucleotide, a phosphorothioate (PS) bond between nucleotides, a 2′-fluoro (2′-F) modified nucleotide, or a combination thereof. For example, the at least one modification can comprise a 2′-O-methyl (2′-O-Me) modified nucleotide. Alternatively, or additionally, the at least one modification can comprise a phosphorothioate (PS) bond between nucleotides. Alternatively, or additionally, the at least one modification can comprise a 2′-fluoro (2′-F) modified nucleotide. In one example, a guide RNA described herein comprises one or more 2′-O-methyl (2′-O-Me) modified nucleotides and one or more phosphorothioate (PS) bonds between nucleotides.

The modifications can occur anywhere in the guide RNA. As one example, the guide RNA comprises a modification at one or more of the first five nucleotides at the 5′ end of the guide RNA, the guide RNA comprises a modification at one or more of the last five nucleotides of the 3′ end of the guide RNA, or a combination thereof. For example, the guide RNA can comprise phosphorothioate bonds between the first four nucleotides of the guide RNA, phosphorothioate bonds between the last four nucleotides of the guide RNA, or a combination thereof. Alternatively, or additionally, the guide RNA can comprise 2′-O-Me modified nucleotides at the first three nucleotides at the 5′ end of the guide RNA, can comprise 2′-O-Me modified nucleotides at the last three nucleotides at the 3′ end of the guide RNA, or a combination thereof.

Another chemical modification that has been shown to influence nucleotide sugar rings is halogen substitution. For example, 2′-fluoro (2′-F) substitution on nucleotide sugar rings can increase oligonucleotide binding affinity and nuclease stability. Abasic nucleotides refer to those which lack nitrogenous bases. Inverted bases refer to those with linkages that are inverted from the normal 5′ to 3′ linkage (i.e., either a 5′ to 5′ linkage or a 3′ to 3′ linkage).

An abasic nucleotide can be attached with an inverted linkage. For example, an abasic nucleotide may be attached to the terminal 5′ nucleotide via a 5′ to 5′ linkage, or an abasic nucleotide may be attached to the terminal 3′ nucleotide via a 3′ to 3′ linkage. An inverted abasic nucleotide at either the terminal 5′ or 3′ nucleotide may also be called an inverted abasic end cap.

In one example, one or more of the first three, four, or five nucleotides at the 5′ terminus, and one or more of the last three, four, or five nucleotides at the 3′ terminus are modified. The modification can be, for example, a 2′-O-Me, 2′-F, inverted abasic nucleotide, phosphorothioate bond, or other nucleotide modification well known to increase stability and/or performance.

In another example, the first four nucleotides at the 5′ terminus, and the last four nucleotides at the 3′ terminus can be linked with phosphorothioate bonds.

In another example, the first three nucleotides at the 5′ terminus, and the last three nucleotides at the 3′ terminus can comprise a 2′-O-methyl (2′-O-Me) modified nucleotide. In another example, the first three nucleotides at the 5′ terminus, and the last three nucleotides at the 3′ terminus comprise a 2′-fluoro (2′-F) modified nucleotide. In another example, the first three nucleotides at the 5′ terminus, and the last three nucleotides at the 3′ terminus comprise an inverted abasic nucleotide.

Multiple gRNAs can be incorporated into a recombinant pseudotyped virus or cell-derived nanovesicle disclosed herein. The gRNAs can be the same or different gRNAs or can target the same gene or different genes. In some embodiments, 1, 2, 3, 4, 5 or more guide RNAs are incorporated into a recombinant pseudotyped virus or cell-derived nanovesicle disclosed herein.

When a gRNA is provided in the form of DNA, the gRNA after being delivered to the target cell can be transiently, conditionally, or constitutively expressed in the cell. DNAs encoding gRNAs can be stably integrated into the genome of the cell and operably linked to a promoter active in the cell. Alternatively, DNAs encoding gRNAs can be operably linked to a promoter in an expression construct. For example, the DNA encoding the gRNA can be in the same recombinant pseudotyped virus or cell-derived nanovesicle comprising a heterologous nucleic acid, such as a nucleic acid encoding a Cas protein. Alternatively, it can be in a recombinant pseudotyped virus or cell-derived nanovesicle that is separate from the recombinant pseudotyped virus or cell-derived nanovesicle comprising the nucleic acid encoding the Cas protein. Promoters that can be used in such expression constructs include promoters active, for example, in one or more of a eukaryotic cell, a human cell, a non-human cell, a mammalian cell, a non-human mammalian cell, a rodent cell, a mouse cell, a rat cell, a pluripotent cell, an embryonic stem (ES) cell, an adult stem cell, a developmentally restricted progenitor cell, an induced pluripotent stem (iPS) cell, or a one-cell stage embryo. Such promoters can be, for example, conditional promoters, inducible promoters, constitutive promoters, or tissue-specific promoters. Such promoters can also be, for example, bidirectional promoters. Specific examples of suitable promoters include an RNA polymerase III promoter, such as a human U6 promoter, a rat U6 polymerase III promoter, or a mouse U6 polymerase III promoter.

In some embodiments, a molecular cargo may comprise a polynucleotide molecule(s) which is capable of modifying expression of one more genes (e.g., inhibiting gene expression and/or translation, modulating RNA splicing or inducing exon skipping) in a target cell. In some embodiments, the polynucleotide molecule may be an interfering nucleic acid molecule, e.g., an siRNA, an shRNA, a miRNA, or an antisense oligonucleotide (ASO), that targets, e.g., an RNA (e.g., an mRNA).

In certain embodiments, interfering nucleic acid molecules that selectively target and inhibit the activity or expression of a product (e.g., an mRNA product) of a targeted gene. An interfering nucleic acid molecule may inhibit the expression or activity of a product (e.g., an mRNA product) of at least one targeted gene by at least 5%, at least 10%, at least 15%, at least 20%, at least 25%, at least 30%, at least 35%, at least 40%, at least 45%, at least 50%, at least 60%, at least 65%, at least 70%, at least 75%, at least 80%, at least 85%, at least 90%, at least 95%, or 100%. An agent disclosed herein may comprise a nucleobase sequence that is at least at least 80%, at least 85%, at least 90%, at least 95%, or 100% complementarity to a product (e.g., an mRNA product) of at least targeted gene.

Interfering nucleic acid molecules described herein can contain RNA bases, non-RNA bases or a mixture of RNA bases and non-RNA bases. For example, interfering nucleic acid molecules described herein can be primarily composed of RNA bases or modified RNA bases, but also contain DNA bases, modified DNA bases, and/or non-naturally occurring nucleotides. The term “ribonucleotide” or “nucleotide” can, in the case of a modified RNA or nucleotide surrogate, also refer to a modified nucleotide, or surrogate replacement moiety at one or more positions.

In some embodiments, the interfering nucleic acid molecule can be a small interfering RNAs (siRNA), also known as short interfering RNA or silencing RNA. siRNAs are a class of double-stranded RNA molecules, typically about 20-25 base pairs in length that target nucleic acids (e.g., mRNAs) for degradation via the RNA interference (RNAi) pathway in cells. Such siRNA molecules typically include a region of sufficient homology to the target region, and are of sufficient length in terms of nucleotides, such that the siRNA molecules down-regulate target nucleic acid. It is not necessary that there be perfect complementarity between the siRNA molecule and the target, but the correspondence must be sufficient to enable the siRNA molecule to direct sequence-specific silencing, such as by RNAi cleavage of the target RNA. In some embodiments, the sense strand need only be sufficiently complementary with the antisense strand to maintain the overall double-strand character of the molecule.

Specificity of siRNA molecules may be measured via the binding of the antisense strand of the molecule to its target RNA. Effective siRNA molecules are often fewer than 30 to 35 base pairs in length, e.g., to prevent stimulation of non-specific RNA interference pathways in the cell by way of the interferon response, however longer siRNA may also be effective. In various embodiments, the siRNA molecules are 5, 6, 7, 8, 9, 10, 11, 12, 13, 14, 15, 16, 17, 18, 19, 20, 21, 22, 23, 24, 25, 26, 27, 28, 29, or 30 base pairs in length. In various embodiments, the siRNA molecules are about 35 to about 70 more base pairs in length. In some embodiments, the siRNA molecules are more than 70 base pairs in length. In some embodiments, the siRNA molecules are 8 to 40 base pairs in length, 10 to 20 base pairs in length, 10 to 30 base pairs in length, 15 to 20 base pairs in length, 19 to 23 base pairs in length, 21 to 24 base pairs in length. In some embodiments, the sense and antisense strands of the siRNA molecules are each independently about 19 to about 24 nucleotides in length. In some embodiments, the sense strand of an siRNA molecule is 23 nucleotides in length and the antisense strand is 21 nucleotides in length. In some embodiments, both the sense strand and the antisense strand of an siRNA molecule are 21 nucleotides in length.

After selection of a suitable target RNA sequence, siRNA molecules that comprise a nucleotide sequence complementary to all or a portion of the target sequence, i.e., an antisense sequence, may be designed and prepared. In some embodiments, the siRNA molecule may be single-stranded (i.e., a ssRNA molecule comprising just an antisense strand) or double stranded (i.e., a dsRNA molecule comprising an antisense strand and a complementary sense strand that hybridizes to form the dsRNA). In various embodiments, the siRNA molecules may comprise a duplex, asymmetric duplex, hairpin or asymmetric hairpin secondary structure, comprising self-complementary sense and/or antisense strands.

In various embodiments, the antisense strand of the siRNA molecule is 5, 6, 7, 8, 9, 10, 11, 12, 13, 14, 15, 16, 17, 18, 19, 20, 21, 22, 23, 24, 25, 26, 27, 28, 29, or 30 nucleotides in length. In various embodiment, the antisense strand of the siRNA molecule is about 35 to about 70 nucleotides in length. In various embodiment, the antisense strand of the siRNA molecule is more than 70 nucleotides in length. In some embodiments, the antisense strand is 8 to 40 nucleotides in length, 10 to 20 nucleotides in length, 10 to 30 nucleotides in length, 15 to 20 nucleotides in length, 19 to 23 nucleotides in length, or 21 to 24 nucleotides in length.

In some embodiments, the sense strand of the siRNA molecule is 5, 6, 7, 8, 9, 10, 11, 12, 13, 14, 15, 16, 17, 18, 19, 20, 21, 22, 23, 24, 25, 26, 27, 28, 29, or 30 more nucleotides in length. In various embodiments, the sense strand of the siRNA molecule is about 30 to about 70 nucleotides in length. In various embodiments, the sense strand of the siRNA molecule more than 70 nucleotides in length. In some embodiments, the sense strand is 8 to 40 nucleotides in length, 10 to 20 nucleotides in length, 10 to 30 nucleotides in length, 15 to 20 nucleotides in length, 19 to 23 nucleotides in length, 21 to 24 nucleotides in length.

In various embodiments, siRNA molecules can comprise an antisense strand comprising a region of complementarity to a target region in a target mRNA. In some embodiments, the region of complementarity is at least 70%, at least 75%, at least 80%, at least 85%, at least 90%, at least 91%, at least 92%, at least 93%, at least 94%, at least 95%, at least 96%, at least 97%, at least 98%, at least 99% or 100% complementary to a target region in a target mRNA. In some embodiments, the target region may comprise a region of consecutive nucleotides in the target mRNA. In some embodiments, it may not be requisite for a region of complementarity to be 100% complementary to that of its target to be specifically hybridizable or specific for a target RNA sequence.

In some embodiments, siRNA molecules disclosed herein may comprise an antisense strand that comprises a region of complementarity to a target RNA sequence and the region of complementarity is in the range of 8 to 20, 8 to 35, 8 to 45, or 10 to 50, or 5 to 55, or 5 to 40 nucleotides in length. In some embodiments, a region of complementarity is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, the region of complementarity is complementary with at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, or more consecutive nucleotides of a target RNA sequence. In some embodiments, siRNA molecules comprise an antisense strand having a nucleotide sequence that contains no more than 1, 2, 3, 4, 5, 6, 7, 8, 9, or 10 base mismatches compared to the portion of the consecutive nucleotides of target RNA sequence. In some embodiments, siRNA molecules comprise a nucleotide sequence that has up to 3 mismatches over 15 bases, or up to 4 mismatches over 10 bases with a target sequence. In some embodiments, siRNA molecules comprises an antisense strand having a nucleotide sequence that has up 0, 1, 2, or 3 mismatches over 15-22 bases with a target sequence. In some embodiments, siRNA molecules comprises an antisense strand having a nucleotide sequence that has 0, 1, or 2 mismatches over 15-22 bases with a target sequence. In some embodiments, siRNA molecules comprises an antisense strand having a nucleotide sequence that has 0 or 1 mismatch over 15-22 bases with a target sequence. In some embodiments, siRNA molecules comprises an antisense strand having a nucleotide sequence that has 0 mismatches over 15-22 bases with a target sequence.

In various embodiments, siRNA molecules may comprise an antisense strand comprising a nucleotide sequence that is at least 70%, at least 75%, at least 85%, at least 90%, at least 95%, or 100% complementary to the target RNA sequence of the antisense oligonucleotides disclosed herein. In some embodiments, siRNA molecules comprise an antisense strand comprising a nucleotide sequence that is at least 70%, at least 75%, at least 85%, at least 90%, at least 95%, or 100% identical to any of the antisense oligonucleotides provided herein. In some embodiments, siRNA molecules comprise an antisense strand comprising at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, or more consecutive nucleotides of any of the antisense oligonucleotides provided herein.

In some embodiments, double-stranded siRNA can comprise sense and anti-sense RNA strands that are different lengths or the same length. In some embodiments, double-stranded siRNA molecules may also be generated from a single oligonucleotide in a stem-loop structure. The self-complementary sense and antisense regions of the siRNA molecule having a stem-loop structure may be linked by means of a nucleic acid based or a non-nucleic acid-based linker. In some embodiments, an siRNA having a stem-loop structure comprises a circular single-stranded RNA having two or more loop structures and a stem comprising self-complementary sense and antisense strands. In some embodiments, the circular RNA may be processed in vivo or in vitro to produce an active siRNA molecule which may be capable of mediating RNAi. Small hairpin RNA (shRNA) molecules are therefore also contemplated in the present disclosure. Such molecules may comprise a specific antisense sequence together with the reverse complement (sense) sequence, which may be separated by a spacer or loop sequence in some instances. A reverse complement described herein may comprise a sequence that is a complement sequence of a reference sequence, wherein the complement sequence is written in the reverse orientation. Due to codon usage redundancy, a reverse complement can diverge from a reference sequence that encodes the same polypeptide. As used herein, “reverse complement” also includes sequences that are, e.g., at least 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identical to the reverse complement sequence of a reference sequence. Cleavage of the spacer or loop can provide a single-stranded RNA molecule and its reverse complement, such that they may anneal to form a dsRNA molecule. In various embodiments, additional optional processing steps may result in removal or addition of 1, 2, 3, 4, 5 or more nucleotides from the 3′ end and/or the 5′ end of one or both strands. A spacer may be of a suitable length to allow the antisense and sense sequences to anneal and form a double-stranded structure or stem prior to cleavage of the spacer. In certain embodiments subsequent optional processing steps may result in removal or addition of 1, 2, 3, 4, 5 or more nucleotides from the 3′ end and/or the 5′ end of one or both strands. In some embodiments, a spacer sequence can be an unrelated nucleotide sequence that may be, e.g., situated between two complementary nucleotide sequence regions that, when annealed into a double-stranded nucleic acid, can comprise a shRNA.

The length of the siRNA molecules can vary from about 10 to about 120 nucleotides depending on the type of siRNA molecule being designed. Generally, between about 10 and about 55 of these nucleotides may be complementary to the RNA target sequence. For instance, when the siRNA is a double-stranded siRNA or single-stranded siRNA, the length can vary from about 10 to about 55 nucleotides, whereas when the siRNA is a shRNA or circular molecule, the length can vary from about 30 nucleotides to about 110 nucleotides.

In various embodiments, an siRNA molecule can comprise a 3′ overhang at one end of the molecule. In some embodiments, the other end can be blunt-ended or may also comprise an overhang (e.g., 5′ and/or 3′). When the siRNA molecule comprises an overhang at both ends of the molecule, the length of the overhangs may be different or the same. In some embodiments, an siRNA molecule described herein may comprises 3′ overhangs of about 1 to about 3 nucleotides on both ends of the molecule. In some embodiments, the siRNA molecule comprises 3′ overhangs of about 1 to about 3 nucleotides on both the sense strand and the antisense strand. In some embodiments, the siRNA molecule comprises 3′ overhangs of about 1 to about 3 nucleotides on the antisense strand. In some embodiments, the siRNA molecule may comprise 3′ overhangs of about 1 to about 3 nucleotides on the sense strand.

In various embodiments, the siRNA molecule comprises one or more modified nucleotides (e.g., 1, 2, 3, 4, 5, 6, 7, 8, 9, 10, 11, 12, 13, 14, 15 or more). In some embodiments, all of the nucleotides of the sense strand and/or the antisense strand of the siRNA molecule are modified. In certain embodiments, the siRNA molecule can comprise one or more modified nucleotides and/or one or more modified internucleotide linkages. In some embodiments, the siRNA molecule may comprise modified internucleotide linkages at the first and second internucleoside linkages at the 5′ end of the siRNA molecule sense strand. In some embodiments, the siRNA molecule may comprise modified internucleotide linkages at the first and second internucleoside linkages at the 5′ and 3′ ends of the siRNA molecule antisense strand. In some embodiments, the siRNA molecule may comprise modified internucleotide linkages at the first and second internucleoside linkages at the 5′ end of the siRNA molecule sense strand and at the first and second internucleoside linkages at the 5′ and 3′ ends of the siRNA molecule antisense strand.

In some embodiments, the modified nucleotide may comprise a modified sugar moiety (e.g., a 2′ modified nucleotide). In some embodiments, the siRNA molecule can comprise one or more 2′ modified nucleotides, e.g., a 2′-deoxy, 2′-fluoro (2′-F), 2′-O-methyl (2′-O-Me), 2′-O-methoxyethyl (2′-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA). In various embodiments, each nucleotide of the siRNA molecule can a modified nucleotide (e.g., a 2′-modified nucleotide). In some embodiments, the siRNA molecule may comprise one or more phosphorodiamidate morpholinos. In some embodiments, each nucleotide of the siRNA molecule consists of a phosphorodiamidate morpholino.

In various embodiments, the siRNA molecule may comprise a phosphorothioate or other modified internucleotide linkage. In various embodiments, the siRNA molecule may comprise, e.g., a phosphorothioate internucleoside linkage(s). In some embodiments, the siRNA molecule may comprise a phosphorothioate internucleoside linkage(s) between two or more nucleotides. In some embodiments, the siRNA molecule may comprise a phosphorothioate internucleoside linkage(s) between all nucleotides. In some embodiments, the siRNA molecule may comprise modified internucleotide linkages at the first, second, and/or third internucleoside linkage at the 5′ or 3′ end of the siRNA molecule. In some embodiments, the siRNA molecule may comprise modified internucleotide linkages at the first and second internucleoside linkages at the 5′ and/or 3′ end of the siRNA molecule. In some embodiments, the siRNA molecule may comprise modified internucleotide linkages at the first and second internucleoside linkages at the 5′ end of the siRNA molecule sense strand. In some embodiments, the siRNA molecule may comprise modified internucleotide linkages at the first and second internucleoside linkages at the 5′ and 3′ ends of the siRNA molecule antisense strand. In some embodiments, the siRNA molecule may comprise modified internucleotide linkages at the first and second internucleoside linkages at the 5′ end of the siRNA molecule sense strand and at the first and second internucleoside linkages at the 5′ and 3′ ends of the siRNA molecule antisense strand. In some embodiments, the siRNA molecule may comprise modified internucleotide linkages at the first internucleoside linkage at the 5′ and 3′ ends of the siRNA molecule sense strand, at the first, second, and third internucleoside linkages at the 5′ end of the siRNA molecule antisense strand, and at the first internucleoside linkage at the 3′ end of the siRNA molecule antisense strand.

In various embodiments, the modified internucleotide linkages may comprise phosphorus-containing linkages. In some embodiments, phosphorus-containing linkages which may be used in the practice of the present disclosure include, without limitation, chiral phosphorothioates, phosphorothioates, phosphorodithioates, aminoalkylphosphotriesters, phosphotriesters, methyl and other alkyl phosphonates comprising 3′alkylene phosphonates and chiral phosphonates, phosphoramidates comprising 3′-amino phosphoramidate and aminoalkylphosphoramidates, phosphinates, thionoalkylphosphonates, thionophosphoramidates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′.

Any of the various modified formats or chemistries of siRNA molecules disclosed herein may be combined together. For example, without limitation, 1, 2, 3, 4, 5, 6, 7, 8, 9, or 10 or more different types of modifications may be included within the same siRNA molecule.

In various embodiments, the antisense strand may comprise one or more modified nucleotides (e.g., 1, 2, 3, 4, 5, 6, 7, 8, 9, 10, 11, 12, 13, 14, 15 or more). In some embodiments, the antisense strand may comprise one or more modified nucleotides and/or one or more modified internucleotide linkage(s). In some embodiments, the modified nucleotide may comprise a modified sugar moiety (e.g., a 2′ modified nucleotide). In some embodiments, the antisense strand comprises one or more 2′ modified nucleotides, e.g., a 2′-deoxy, 2′-fluoro (2′-F), 2′-O-methyl (2′-O-Me), 2′-O-methoxyethyl (2′-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA). In various embodiments, each nucleotide of the antisense strand can be a modified nucleotide (e.g., a 2′-modified nucleotide). In some embodiments, the antisense strand may comprise one or more phosphorodiamidate morpholinos. In some embodiments, the antisense strand consists of a phosphorodiamidate morpholino oligomer (PMO).

In some embodiments, antisense strand contains a phosphorothioate or other modified internucleotide linkage. In some embodiments, the antisense strand may comprise phosphorothioate internucleoside linkage(s). In some embodiments, the antisense strand may comprise phosphorothioate internucleoside linkage(s) between two or more nucleotides. In some embodiments, the antisense strand may comprise phosphorothioate internucleoside linkage(s) between all nucleotides. In some embodiments, the antisense strand may comprise modified internucleotide linkages at the first, second, and/or third nucleotide at the 5′ or 3′ end of the antisense strand. In some embodiments, the antisense strand may comprise modified internucleotide linkages at the first and second nucleotide positions (e.g., between the first and second and between the second and third nucleotides) at the 5′ and 3′ ends of the antisense strand.

Any of the modified formats or chemistries of the antisense strand disclosed herein may be combined together. For example, without limitation, 1, 2, 3, 4, 5, 6, 7, 8, 9, or 10 or more different types of modifications may be included within the same antisense strand.

In some embodiments, the sense strand comprises one or more modified nucleotides (e.g., 1, 2, 3, 4, 5, 6, 7, 8, 9, 11, 12, 13, 14, 15 or more). In some embodiments, the antisense strand may comprise one or more modified nucleotides and/or one or more modified internucleotide linkage(s). In some embodiments, the modified nucleotide may comprise a modified sugar moiety (e.g., a 2′ modified nucleotide). In some embodiments, the antisense strand comprises one or more 2′ modified nucleotides, e.g., a 2′-deoxy, 2′-fluoro (2′-F), 2′-O-methyl (2′-O-Me), 2′-O-methoxyethyl (2′-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA). In various embodiments, each nucleotide of the antisense strand can be a modified nucleotide (e.g., a 2′-modified nucleotide). In some embodiments, the antisense strand may comprise one or more phosphorodiamidate morpholinos. In some embodiments, the antisense strand consists of a phosphorodiamidate morpholino oligomer (PMO).

In some embodiments, the sense strand contains a phosphorothioate or other modified internucleotide linkage. In some embodiments, the sense strand may comprise phosphorothioate internucleoside linkage(s). In some embodiments, the sense strand may comprise phosphorothioate internucleoside linkage(s) between two or more nucleotides. In some embodiments, the sense strand may comprise phosphorothioate internucleoside linkages between all nucleotides. For example, in some embodiments, the sense strand comprises modified internucleotide linkages at the first, second, and/or third nucleotide at the 5′ or 3′ end of the sense strand. In some embodiments, the sense strand may comprise modified internucleotide linkages at the first and second nucleotide positions (e.g., between the first and second and between the second and third nucleotides) at the 5′ end of the sense strand.

In various embodiments, the modified internucleotide linkages may comprise phosphorus-containing linkages of the sense strand. In some embodiments, phosphorus-containing linkages which may be used in the practice of the present disclosure include, without limitation, chiral phosphorothioates, phosphorothioates, phosphorodithioates, aminoalkylphosphotriesters, phosphotriesters, methyl and other alkyl phosphonates comprising 3′alkylene phosphonates and chiral phosphonates, phosphoramidates comprising 3′-amino phosphoramidate and aminoalkylphosphoramidates, phosphinates, thionoalkylphosphonates, thionophosphoramidates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′.

Any of the modified chemistries or formats of the sense strand described herein can be combined together. For example, without limitation, 1, 2, 3, 4, 5, 6, 7, 8, 9, or 10 or more different types of modifications may be included within the same sense strand.

In various embodiments, the antisense and/or sense strand of the siRNA molecule may comprise one or more modifications capable of enhancing or reducing, e.g., RNA-induced silencing complex (RISC) loading. In some embodiments, the antisense strand of the siRNA molecule may comprise one or more modifications capable of enhancing RISC loading. In various embodiments, the sense strand of the siRNA molecule may comprise one or more modifications capable of reducing RISC loading and/or reducing off-target effects. In various embodiments, the antisense strand of the siRNA molecule may comprise a 2′-O-methoxyethyl (2′-MOE) modification. In some embodiments, the addition of the 2′-O-methoxyethyl (2′-MOE) group, e.g., at the cleavage site may improve the silencing activity and/or specificity of siRNAs, e.g., by facilitating the oriented RNA-induced silencing complex (RISC) loading of the modified strand, e.g., as disclosed in Song et al., (2017) Mol Ther Nucleic Acids 9:242-250, incorporated herein by reference in its entirety. In various embodiments, the antisense strand of the siRNA molecule may comprise a 2′-O-Me-phosphorodithioate modification. In some embodiment, the 2′-O-Me-phosphorodithioate modification may increase RISC loading, e.g., as disclosed in Wu et al., (2014) Nat Commun 5:3459, incorporated herein by reference in its entirety.

In various embodiments, the sense strand of the siRNA molecule may comprise a 5′-nitroindole modification. In some embodiments, the 5′-nitroindole modification may decrease the RNAi potency of the sense strand and/or reduces off-target effects. In various embodiments, the sense strand may comprise a 2′-O-methyl (2′-O-Me) modification. In some embodiments, the 2′-O-Me modification may reduce RISC loading and/or the off-target effects of the sense strand. In various embodiments, the sense strand of the siRNA molecule may be fully substituted with morpholino, 2′-MOE and/or 2′-O-Me residues, and may not be recognized by RISC, e.g., as disclosed in Kole et al., (2012) Nature reviews. Drug Discovery 11(2): 125-140, incorporated herein by reference in its entirety.

In various embodiments, the sense strand of the siRNA molecule may comprise a 5′-morpholino modification. In various embodiments, the 5′-morpholino modification may reduce RISC loading of the sense strand and/or improves RNAi activity and/or antisense strand selection, e.g., as disclosed in Kumar et al., (2019) Chem Commun (Camb) 55(35):5139-5142, incorporated herein by reference in its entirety. In various embodiments, the sense strand of the siRNA molecule may be modified, for example, with a synthetic RNA-like high affinity nucleotide analogue called Locked Nucleic Acid (LNA) that may reduce RISC loading of the sense strand and promote antisense strand incorporation into RISC, e.g., as disclosed in Elman et al., (2005) Nucleic Acids Res. 33(1): 439-447, incorporated herein by reference in its entirety. In various embodiments, the sense strand of the siRNA molecule may comprise a 5′ unlocked nucleic acid (UNA) modification. In various embodiments, the 5′ unlocked nucleic acid (UNA) modification may reduce RISC loading of the sense strand and/or improve silencing capability of the antisense strand, e.g., as disclosed in Snead et al., (2013) Mol Ther Nucleic Acids 2(7):e103, incorporated herein by reference in its entirety.

In some embodiments, the antisense strand of the siRNA molecule may comprise a 2′-MOE modification and/or the sense strand may comprise a 2′-O-Me modification.

In addition, an siRNA molecule may be modified or include nucleoside surrogates. Single stranded regions of an siRNA molecule may be modified or include nucleoside surrogates, e.g., the unpaired region or regions of a hairpin structure, e.g., a region which links two complementary regions, can have modifications or nucleoside surrogates. Modification to stabilize one or more 3′- or 5′-termini of an siRNA molecule, e.g., against exonucleases, or to favor the antisense siRNA agent to enter into RISC are also useful. Modifications can include C3 (or C6, C7, C12) amino linkers, thiol linkers, carboxyl linkers, non-nucleotidic spacers (e.g., C3-C12 (e.g., C3, C6, C9, C12), abasic, tri ethylene glycol, hexaethylene glycol), biotin or fluorescein reagents that come as phosphoramidites and that have another DMT-protected hydroxyl group, allowing multiple couplings during RNA synthesis.

In some embodiments, the sense strand is 23 nucleotides in length and the antisense strand is 21 nucleotides in length. In some embodiments, the sense strand is 23 nucleotides in length and the antisense strand is 21 nucleotides in length, wherein the 3′ and 5′ terminal nucleotide positions of the sense strand are inverted abasic residues. The sense strand 3′ and 5′ terminal inverted abasic residues may be overhangs. The inverted abasic residues may be linked via a 3′-3′ phosphodiester linkage. In some embodiments, the antisense strand of the siRNA molecule contains 1-2 phosphorothioate linkages at the 3′ and/or 5′ ends. In some embodiments, the antisense strand contains two or three phosphorothioate internucleotide linkages at the 5′-terminus and 1 phosphorothioate internucleotide linkage at the 3′-terminus. The siRNA molecule may be linked to a targeting moiety at the 5′ or 3′ end of the sense strand.

In some embodiments, the sense strand is 21 nucleotides in length and the antisense strand is 23 nucleotides in length, wherein the antisense strand contains a 2 nucleobase 3′ overhang. In some embodiments, the antisense strand of the siRNA molecule contains 1-3 phosphorothioate linkages at the 3′ and 5′ ends and the sense strand of the siRNA molecule contains 1-2 phosphorothioate linkages at the 5′ end. In some embodiments, the antisense strand of the siRNA molecule contains 2-3 phosphorothioate linkages at the 5′ end and 2 phosphorothioate linkages at the 3′, and the sense strand of the siRNA molecule contains 2 phosphorothioate linkages at the 5′ end. The siRNA molecule may be linked to a targeting moiety at the 5′ or 3′ end of the sense strand.

In some embodiments, the interfering nucleic acid molecule is a short hairpin RNA (shRNA). A “small hairpin RNA “or “short hairpin RNA” or “shRNA” described herein may include a short RNA sequence that makes a tight hairpin turn that can be used to silence gene expression via RNA interference. The shRNAs provided herein may be chemically synthesized or transcribed from a transcriptional cassette in a DNA plasmid. The shRNA hairpin structure may be cleaved by the cellular machinery into siRNA, which is then bound to the RNA-induced silencing complex (RISC).

Non-limiting examples of shRNAs include a double-stranded polynucleotide molecule assembled from a single-stranded molecule, where the sense and antisense regions are linked by a nucleic acid-based or non-nucleic acid-based linker; and a double-stranded polynucleotide molecule with a hairpin secondary structure having self-complementary sense and antisense regions. In some embodiments, the sense and antisense strands of the shRNA are linked by a loop structure comprising from about 1 to about 25 nucleotides, from about 2 to about 20 nucleotides, from about 4 to about 15 nucleotides, from about 5 to about 12 nucleotides, or 1, 2, 3, 4, 5, 6, 7, 8, 9, 10, 11, 12, 13, 14, 15, 16, 17, 18, 19, 20, 21, 22, 23, 24, 25, or more nucleotides.

In some embodiments, the interfering nucleic acid molecule is a microRNA (miRNA). miRNAs represent a large group of small RNAs produced naturally in organisms, some of which regulate the expression of target genes. miRNAs are short hairpin RNAs about 18 to about 25 nucleotides in length that function in RNA silencing and post-translational regulation of gene expression. Typically, miRNAs are generated from large RNA precursors (termed pri-miRNAs) that are processed in the nucleus into approximately 70 nucleotide pre-miRNAs, which fold into imperfect stem-loop structures. These pre-miRNAs typically undergo an additional processing step within the cytoplasm where mature miRNAs of 18-25 nucleotides in length are excised from one side of the pre-miRNA hairpin by an RNase III enzyme, Dicer. miRNAs are not translated into proteins, but instead bind to specific messenger RNAs, thereby blocking translation. In some embodiments, miRNAs base-pair imprecisely with their targets to inhibit translation.

miRNAs as described herein can include pri-miRNA, pre-miRNA, mature miRNA or fragments of variants thereof that retain the biological activity of mature miRNA. In some embodiments, the size range of the miRNA can be from 21 nucleotides to 170 nucleotides. In one embodiment, the size range of the miRNA is from 70 to 170 nucleotides in length. In another embodiment, mature miRNAs of from 21 to 25 nucleotides in length can be used.

In certain embodiments, the interfering nucleic acid molecule is an antisense oligonucleotide (ASO). An ASO can down regulate a target by inducing RNase H endonuclease cleavage of a target RNA, by steric hindrance of ribosomal activity, by inhibiting 5′ cap formation, or by altering splicing. An ASO can be, but is not limited to, a gapmer or a morpholino. An antisense oligonucleotide typically comprises a short nucleotide sequence which is substantially complementary to a target nucleotide sequence in a pre-mRNA molecule, heterogeneous nuclear RNA (hnRNA) or mRNA molecule. The degree of complementarity (or substantial complementarity) of the antisense sequence is preferably such that a molecule comprising the antisense sequence can form a stable double stranded hybrid with the target nucleotide sequence in the RNA molecule under physiological conditions. Antisense oligonucleotides are often synthetic and chemically modified.

Antisense oligonucleotides may be 100% complementary to the target sequence, or may include mismatches, e.g., to improve selective targeting of allele containing the disease-associated mutation, as long as a heteroduplex formed between the oligonucleotide and target sequence is sufficiently stable to withstand the action of cellular nucleases and other modes of degradation which may occur in vivo. Hence, certain oligonucleotides may have about or at least about 70% sequence complementarity, e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence complementarity, between the oligonucleotide and the target sequence. Oligonucleotide backbones that are less susceptible to cleavage by nucleases are discussed herein. Mismatches, if present, are typically less destabilizing toward the end regions of the hybrid duplex than in the middle. The number of mismatches allowed will depend on the length of the oligonucleotide, the percentage of G:C base pairs in the duplex, and the position of the mismatch(es) in the duplex, according to well understood principles of duplex stability.

In some embodiments, an interfering nucleic acid molecule described herein is a gapmer. A “gapmer” is oligonucleotide comprising an internal region having a plurality of nucleosides that support RNase H cleavage positioned between external regions having one or more nucleosides, wherein the nucleosides comprising the internal region are chemically distinct from the nucleoside or nucleosides comprising the external regions. The internal region may be referred to as the “gap” and the external regions may be referred to as the “wings.” A gapmer can have 5′ and 3′ wings each having 2-6 nucleotides and a gap having 7-12 nucleotides. In some embodiments, a gapmer can have a 3-10-3 configuration or a 5-10-5 configuration.

A gapmer commonly has the formula 5′-X-Y-Z-3′, with X and Z as flanking regions around a gap region Y. In some embodiments, flanking region X of formula 5′-X-Y-Z-3′ is also called X region, flanking sequence X, 5′ wing region X, or 5′ wing segment. In some embodiments, flanking region Z of formula 5′-X-Y-Z-3′ is also called Z region, flanking sequence Z, 3′ wing region Z, or 3′ wing segment. In some embodiments, gap region Y of formula 5′-X-Y-Z-3′ is also called Y region, Y segment, gap-segment Y, gap segment, or gap region. In some embodiments, each nucleoside in the gap region Y is a 2′-deoxyribonucleoside, and neither the 5′ wing region X or the 3′ wing region Z comprises any 2′-deoxyribonucleosides.

In some embodiments, a gapmer is 10-50 nucleosides in length. For example, a gapmer may be 10-50, 10-45, 10-40, 10-35, 10-30, 10-25, 10-20, 10-15, 15-40, 15-35, 15-30, 15-25, 15-20, 20-40, 20-35, 20-30, 20-25, 25-40, 25-35, 25-30, 30-40, 30-35, or 35-40 nucleosides in length. In some embodiments, a gapmer is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleosides in length. In some embodiments, a gapmer is about 16 to about 20 nucleosides in length. In some embodiments, a gapmer is 16 nucleotides in length. In some embodiments, a gapmer is 20 nucleotides in length.

Interfering nucleic acid molecules can be prepared, for example, by chemical synthesis, in vitro transcription, or digestion of long dsRNA by RNase III or Dicer. These can be introduced into cells by e.g., transfection, electroporation.

In some embodiments, a molecular cargo, e.g., a polynucleotide molecule described herein, may comprise a ribozyme (ribonucleic acid enzyme). Without wishing to be bound by theory, a ribozyme is a molecule, commonly an RNA molecule, that is capable of performing specific biochemical reactions, akin to the action of protein enzymes. Ribozymes comprise molecules possessing catalytic activities such as, but not limited to, the capacity to cleave at specific phosphodiester linkages in RNA molecules to which they have hybridized, e.g., RNA-containing substrates, lncRNAs, mRNAs, and ribozymes.

Ribozymes may take on one of several physical structures, one such structure is termed “hammerhead”. A hammerhead ribozyme can comprise, e.g., a catalytic core comprising nine conserved bases, two regions complementary to the target RNA flanking regions the catalytic core, and a double-stranded stem and loop structure (stem-loop II). The flanking regions may permit the binding of the ribozyme to the target RNA, in particular, by forming double-stranded stems I and III. Cleavage may occur in trans (cleavage of an RNA substrate other than that containing the ribozyme) or in cis (cleavage of the same RNA molecule that contains the hammerhead motif) adjacent to a specific ribonucleotide triplet by a transesterification reaction from a 3′, 5′-phosphate diester to a 2′, 3′-cyclic phosphate diester. In certain embodiments, this catalytic activity may require the presence of specific, highly conserved sequences in the catalytic region of the ribozyme.

Modifications in ribozyme structure can include the replacement or substitution of non-core portions of the molecule with non-nucleotidic molecules. As a non-limiting example, Ma et al. (Biochem. (1993) 32:1751-1758; Nucleic Acids Res. (1993) 21:2585-2589) replaced the six-nucleotide loop of the TAR ribozyme hairpin with non-nucleotidic, ethylene glycol-related linkers. Thomson et al. (Nucleic Acids Res. (1993) 21:5600-5603) replaced loop II with linear, non-nucleotidic linkers of 13, 17, and 19 atoms in length. Benseler et al. (J. Am. Chem. Soc. (1993) 115:8483-8484) describes hammerhead-like molecules where two of the base pairs of stem II, and all four of the nucleotides of loop II may be replaced with non-nucleoside linkers based on bis(propanediol) phosphate, hexaethylene glycol, bis(triethylene glycol) phosphate, propanediol, or tris(propanediol)bisphosphate.

In some embodiments, the ribozyme polynucleotide described herein can incorporate nucleotide analogs, e.g., to increase the resistance of the oligonucleotide to degradation by nucleases in a cell. The ribozyme may be synthesized in any known manner, e.g., by use of a commercially available synthesizer produced, e.g., by Applied Biosystems, Inc. or Milligen. The ribozyme RNA sequences maybe synthesized conventionally, for example, by using RNA polymerases such as T7 or SP6. The ribozyme may also be produced in recombinant vectors by suitable means.

In some embodiments, internucleotidic phosphorus atoms of the polynucleotide molecules disclosed herein may be chiral, and the properties of the polynucleotides by adjusted based on the configuration of the chiral phosphorus atoms. In some embodiments, appropriate methods may be used to synthesize P-chiral oligonucleotide analogs in a stereocontrolled manner (e.g., as described in Oka N, Wada T, Stereocontrolled synthesis of oligonucleotide analogs containing chiral internucleotidic phosphorus atoms. Chem Soc Rev. 2011 December; 40(12):5829-43, the contents of which are incorporated herein by reference in their entirety). In some embodiments, phosphorothioate-containing oligonucleotides may comprise nucleoside units that can be joined together by either substantially all Rp or substantially all Sp phosphorothioate inter-sugar linkages. In some embodiments, such phosphorothioate oligonucleotides comprising substantially chirally pure inter-sugar linkages may be produced via chemical synthesis or enzymatic approaches, as disclosed, e.g., in U.S. Pat. No. 5,587,261, the contents of which are incorporated herein by reference in their entirety. In some embodiments, chirally controlled polynucleotide molecules described may provide selective cleavage patterns of a target nucleic acid. As a non-limiting example, a chirally controlled polynucleotide molecule may provide single site cleavage within a complementary sequence of a nucleic acid, as disclosed, for example, in US Patent Publication No. 2017/0037399, the contents of which are incorporated herein by reference in their entirety.

In some embodiments, the polynucleotide molecule described herein may be a morpholino-based compound. In some embodiments, the morpholino-based oligomeric compound is a phosphorodiamidate morpholino oligomer (PMO).

In some embodiments, a polynucleotide molecule described herein may comprise an aptamer. An aptamer may comprise any nucleic acid which specifically binds specifically to a target, e.g., protein or nucleic acid in a cell. In some embodiments, the aptamer is a DNA aptamer or an RNA aptamer. In some embodiments, a nucleic acid aptamer may comprise a single-stranded RNA (ssDNA or ssRNA) or DNA. In certain embodiments, a single-stranded nucleic acid aptamer may form loop(s) and/or helice(s) structures. The nucleic acid that forms the nucleic acid aptamer may comprise naturally occurring nucleotides, modified nucleotides with hydrocarbon or PEG linkers inserted between one or more nucleotides, modified nucleotides, naturally occurring nucleotides with hydrocarbon linkers (e.g., an alkylene) or a polyether linker (e.g., a PEG linker) inserted between one or more nucleotides, or a combination of thereof. Aptamers and method of producing aptamers are described in, e.g., U.S. Pat. Nos. 8,318,438, 5,650,275; 5,683,867, 5,670,637; 5,696,249; 5,789,157; 5,843,653; 5,270,163; 5,567,588, 5,864,026; 5,989,823; 6,569,630; and PCT Publication No. WO 99/31275, Lorsch and Szostak, 1996; Jayasena, 1999; each incorporated herein by reference.

In some embodiments, a polynucleotide molecule described herein may be a mixmer or comprise a mixmer sequence pattern. In some embodiments, mixmers can be polynucleotides that comprise both naturally and non-naturally occurring nucleosides or comprise two different types of non-naturally occurring nucleosides commonly in an alternating pattern. Mixmers may have higher binding affinity than unmodified polynucleotides and may be used, in particular, to specifically bind a target molecule, e.g., to block a binding site on the target molecule. In some embodiments, mixmers may not recruit an RNase to a target molecule and hence do not promote cleavage of the target molecule. Such polynucleotides that may be incapable of recruiting, e.g., RNase H have been described, e.g., see WO2007/112753 or WO2007/112754.

In some embodiments, a mixmer disclosed herein may comprise a repeating pattern of naturally occurring nucleosides and nucleoside analogues, or, e.g., one type of nucleoside analogue and a second type of nucleoside analogue. Yet, a mixmer need not comprise a repeating pattern and may instead comprise any arrangement of modified naturally occurring nucleosides and nucleosides or any arrangement of one type of modified nucleoside and a second type of modified nucleoside. Such repeating pattern, may, for example comprise every second or every third nucleoside as a modified nucleoside, e.g., LNA. In certain embodiments, the remaining nucleosides may be naturally occurring nucleosides, e.g., DNA, or may be a 2′ substituted nucleoside analogue, e.g., 2′ fluoro analogues or 2′-MOE, or any other some modified nucleoside(s) disclosed herein. It is understood that the repeating pattern of modified nucleoside, such as LNA units, may be combined with modified nucleoside at fixed positions (e.g., at the 5′ and/or 3′ termini).

In some embodiments, a mixmer may not comprise a region of more than 6. more than 5, more than 4, more than 3, or more than 2 consecutive naturally occurring nucleosides (e.g., DNA nucleosides). In some embodiments, the mixmer may comprise at least a region comprising at least two consecutive modified nucleosides, for example, at least two consecutive LNAs. In some embodiments, the mixmer may comprise at least a region consisting of at least three consecutive modified nucleoside units, e.g., at least three consecutive LNAs.

In some embodiments, the mixmer may not comprise a region of more than 8, more than 7, more than 6, more than 5, more than 4, more than 3, or more than 2 consecutive nucleoside analogues, e.g., LNAs. In some embodiments, LNA units may be replaced with other nucleoside analogues including, but not limited to, those referred to herein.

In some embodiments, mixmers may be designed to comprise a mixture of affinity enhancing modified nucleosides, such as, without limitation, in LNA nucleosides and 2′-O-Me nucleosides. In some embodiments, a mixmer may comprise modified internucleoside linkages (e.g., phosphorothioate internucleoside linkages or other linkages) between at least two, at least three, at least four, at least five, at least six or more nucleosides.

In some embodiments, a mixmer may comprise one or more morpholino nucleosides. In some embodiments, a mixmer may comprise morpholino nucleosides mixed (e.g., in an alternating manner) with one or more other nucleosides (e.g., DNA, RNA nucleosides) or modified nucleosides (e.g., 2′-O-Me nucleosides, LNA).

In some embodiments, mixmers may be useful for splice correcting or exon skipping, for example, as described in Chen S. et al., Molecules 2016, 21, 1582, Touznik A., et al., Scientific Reports, volume 7, Article number: 3672 (2017), the contents of each which are incorporated herein by reference.

A mixmer may be produced using any suitable method. Preparation of mixmers is described in, for example, U.S. Pat. No. 7,687,617, and U.S. Patent Application Publication Nos. US2012/0322851, US2009/0209748, US2009/0298916, US2006/0128646, and US2011/0077288. Additional examples of multimers are described, for example, in U.S. Pat. No. 5,693,773, US Patent Application Publication Nos. 2015/0247141; 2015/0315588; US 2011/0158937; the contents of each of which are incorporated herein by reference in their entireties.

In some embodiments, polynucleotide molecules comprising molecular cargos disclosed herein may comprise multimers (e.g., concatemers) of two or more polynucleotide molecules connected, e.g., by a linker. Polynucleotides in a multimer may be the same or different (e.g., targeting different sites on the same gene different genes or products thereof).

In some embodiments, multimers may comprise two or more polynucleotide molecules linked together by a cleavable linker. In some embodiments, multimers may comprise two or more polynucleotide molecules linked together, e.g., by a non-cleavable linker. In some embodiments, a multimer may comprise 2, 3, 4, 5, 6, 7, 8, 9, 10, 11, 12, 13, 14, 15, 16, 17, 18, 19, 20 or more polynucleotide molecules linked together. In some embodiments, a multimer may comprises 2 to 5, 2 to 10, 4 to 20 or 5 to 30 polynucleotide molecules linked together.

In some embodiments, a multimer may comprises two or more polynucleotide molecules linked in a linear arrangement, e.g., end-to-end. In some embodiments, a multimer may comprises two or more polynucleotide molecules linked end-to-end via a polynucleotide-based linker (e.g., an abasic linker, a poly-dT linker). In some embodiments, a multimer comprises a 3′ end of one polynucleotide linked to a 3′ end of another polynucleotide. In some embodiments, a multimer may comprise a 5′ end of one polynucleotide linked to a 3′ end of another polynucleotide. In some embodiments, a multimer comprises a 5′ end of one polynucleotide linked to a 5′ end of another polynucleotide. In some embodiments, multimers may comprise a branched structure comprising multiple polynucleotides linked together by a branching linker.

In some embodiments, a polynucleotide molecule of the present disclosure can target splicing. In some embodiments, the polynucleotide can targets splicing by inducing exon skipping and restoring the reading frame within a gene. For example, without limitation, the oligonucleotide may induce skipping of an exon encoding a frameshift mutation and/or an exon that encodes a premature stop codon. In some embodiments, a polynucleotide may induce exon skipping by, e.g., blocking spliceosome recognition of a splice site. In some embodiments, a polynucleotide molecule disclosed herein may induce inclusion of an exon by targeting a splice site inhibitory sequence. In some embodiments, the oligonucleotide promotes inclusion of a particular exon. In some embodiments, exon skipping results in a truncated but functional protein compared to the reference protein.

In some embodiments, the polynucleotide molecule described herein may be a messenger RNA (mRNA). mRNAs comprise an open reading frame that can be translated into a polypeptide (i.e., can serve as a substrate for translation by a ribosome and amino-acylated tRNAs). mRNA can comprise a phosphate-sugar backbone including ribose residues or analogs thereof, e.g., 2′-methoxy ribose residues. In some embodiments, the sugars of an mRNA phosphate-sugar backbone consist essentially of ribose residues, 2′-methoxy ribose residues, or a combination thereof. Bases of an mRNA can be modified bases such as pseudouridine, N-1-methyl-pseudouridine, or other naturally occurring or non-naturally occurring bases.

In various embodiments, a polynucleotide disclosed herein may comprise at least one nucleoside, e.g., modified at the 2′ position of the sugar. In some embodiments, all of the nucleosides in the polynucleotide are 2′-modified nucleosides. In some embodiments, a polynucleotide comprises at least one 2′-modified nucleoside.

In various embodiments, a polynucleotide disclosed herein may one or more non-bicyclic 2′-modified nucleosides, e.g., 2′-O-dimethylaminoethyloxyethyl (2′-O-DMAEOE)2′-O-methyl (2′-O-Me), 2′-O-dimethylaminoethyl (2′-O-DMAOE), 2′-O-methoxyethyl (2′-MOE), 2′-deoxy, 2′-O—N-methylacetamido (2′-O-NMA) modified nucleoside, 2′-fluoro (2′-F), 2′-O-aminopropyl (2′-O-AP), or 2′-O-dimethylaminopropyl (2′-O-DMAP).

In some embodiments, a polynucleotide of the present disclosure may comprise one or more 2′-4′ bicyclic nucleosides in which the ribose ring may comprise a bridge moiety, e.g., connecting two atoms in the ring (e.g., connecting the 2′-O atom to the 4′-C atom via an ethylene (ENA) bridge, a methylene (LNA) bridge, or a (S)-constrained ethyl (cEt) bridge).

In various embodiments, the polynucleotide comprises at least one modified nucleoside that results in an increase in Tm of the polynucleotide in a range of 1° C. to 10° C. compared with a polynucleotide that does not have the at least one modified nucleoside. The polynucleotide may have a plurality of modified nucleosides that result in a total increase in Tm of the polynucleotide in a range of 2° C., 3° C., 4° C., 5° C., 6° C., 7° C., 8° C., 9° C., 10° C., 15° C., 20° C., 25° C., 30° C., 35° C., 40° C., 45° C., 50° C., 55° C., 60° C. or more as compared to a polynucleotide which does not have the modified nucleoside.

In some embodiments, the polynucleotide may comprise a mix of nucleosides of different kinds. A polynucleotide may comprise a mix of deoxyribonucleosides or ribonucleosides and 2′-O-Me modified nucleosides. A polynucleotide may comprise a mix of 2′-4′ bicyclic nucleosides and 2′-MOE, 2′-fluoro, or 2′-O-Me modified nucleosides. A polynucleotide may comprise a mix of non-bicyclic 2′-modified nucleosides (e.g., 2′-MOE, 2′-fluoro, or 2′-O-Me) and 2′-4′ bicyclic nucleosides (e.g., LNA, ENA, cEt). A polynucleotide may comprise a mix of 2′-deoxyribonucleosides or ribonucleosides and 2′-fluoro modified nucleosides. A polynucleotide may comprise a mix of 2′-fluoro modified nucleosides and 2′-O-Me modified nucleosides.

In various embodiments, the oligonucleotide may comprise alternating nucleosides of different types. In certain embodiments, the oligonucleotide may comprise alternating deoxyribonucleosides or ribonucleosides and 2′-O-Me modified nucleosides. In certain embodiments, a polynucleotide may comprise alternating 2′-deoxyribonucleosides or ribonucleosides and 2′-fluoro modified nucleosides. In certain embodiments, the oligonucleotide may comprise alternating 2′-fluoro modified nucleosides and 2′-O-Me modified nucleosides. In certain embodiments, the oligonucleotide may comprise alternating 2′-4′ bicyclic nucleosides and 2′-MOE, 2′-fluoro, or 2′-O-Me modified nucleosides. In certain embodiments, the oligonucleotide may comprise alternating non-bicyclic 2′-modified nucleosides (e.g., 2′-MOE, 2′-fluoro, or 2′-O-Me) and 2′-4′ bicyclic nucleosides (e.g., LNA, ENA, cEt).

In various embodiments, a polynucleotide of the present disclosure may comprise one or more abasic residues, a 5-vinylphosphonate modification, and/or one or more inverted abasic residues.

In various embodiments, the oligonucleotide may comprise a phosphorothioate or other modified internucleoside linkage. In various embodiments, the oligonucleotide may comprise phosphorothioate internucleoside linkages. In various embodiments, the oligonucleotide comprises phosphorothioate internucleoside linkages between at least two nucleotides. In various embodiments, the oligonucleotide comprises phosphorothioate internucleoside linkages between all nucleotides. By way of a non-limiting example, in certain embodiments, oligonucleotides comprise modified internucleoside linkages at the first, second, and/or (e.g., and) third internucleoside linkage at the 5′ or 3′ end of the nucleotide sequence.

Non-limiting examples of phosphorus-containing linkages include aminoalkylphosphotriesters phosphorothioates, chiral phosphorothioates, phosphotriesters, phosphorodithioates, methyl and other alkyl phosphonates comprising 3′alkylene phosphonates and chiral phosphonates, phosphinates, phosphoramidates comprising 3′-amino phosphoramidate and aminoalkylphosphoramidates, thionoalkylphosphonates, thionophosphoramidates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′.

In various embodiments, a polynucleotide of the present disclosure may have heteroatom backbones, e.g., or peptide nucleic acid (PNA) backbones (wherein the phosphodiester backbone of the oligonucleotide is replaced with a polyamide backbone, the nucleotides being bound directly or indirectly to the aza nitrogen atoms of the polyamide backbone), morpholino backbones; amide backbones; or MMI or methylene(methylimino) backbones.

Nitrogenous bases can be conventional bases (A, G, C, T, U), analogs thereof (e.g., modified uridines such as 5-methoxyuridine, pseudouridine, or N1-methylpseudouridine, or others); inosine; derivatives of purines or pyrimidines (e.g., N4-methyl deoxyguanosine, deaza- or aza-purines, deaza- or aza-pyrimidines, pyrimidine bases with substituent groups at the 5 or 6 position (e.g., 5-methylcytosine), purine bases with a substituent at the 2, 6, or 8 positions, 2-amino-6-methylaminopurine, 6-O-methylguanine, 4-thio-pyrimidines, 4-amino-pyrimidines, 4-dimethylhydrazine-pyrimidines, and 4-O-alkyl-pyrimidines). Nucleic acids can include one or more “abasic” residues where the backbone includes no nitrogenous base for position(s) of the polymer. A nucleic acid can comprise only conventional RNA or DNA sugars, bases and linkages, or can include both conventional components and substitutions (e.g., conventional nucleosides with 2′ methoxy substituents, or polymers containing both conventional nucleotides and one or more nucleotide analogs). Nucleic acid includes “locked nucleic acid” (LNA), an analogue containing one or more LNA nucleotide monomers with a bicyclic furanose unit locked in an RNA mimicking sugar conformation, which enhance hybridization affinity toward complementary RNA and DNA sequences. RNA and DNA have different sugar moieties and can differ by the presence of uracil or analogs thereof in RNA and thymine or analogs thereof in DNA.

### Additional Nucleotide Sequences

In some embodiments, the recombinant virus disclosed herein may further comprise a nucleotide sequence that is a template for a positive sense transcript encoding one or more molecules, for example, an interferon (IFN) polypeptide, a sodium iodide symporter (NIS) polypeptide, a cytotoxic polypeptide, an antibody, or antibody fragment such as a single chain antibody, a tumor antigen, an immune stimulatory molecule, an immune inhibitory molecule (e.g., an immune combat molecule), or a functional fragment or derivative thereof. In some embodiments, the recombinant virus disclosed herein may further comprise a nucleotide sequence that is a template for a positive sense transcript encoding an interferon (IFN) polypeptide or a functional fragment or derivative thereof.

Any appropriate nucleotide sequence that is a template for a positive sense transcript encoding an IFN polypeptide may be inserted into the genome of a recombinant virus disclosed herein, for example, a recombinant virus comprising a rhabdovirus (e.g., vesiculovirus, including, without limitation, VSV) genome. For example, a nucleotide sequence that is a template for a positive sense transcript encoding an IFN beta polypeptide can be inserted into the genome of a recombinant virus disclosed here. Examples of nucleic acids encoding IFN beta polypeptides include, without limitation, nucleic acid encoding a human IFN beta poly-peptide of the nucleic acid sequence set forth in GenBank® Accession No. NM_002176.2 (GI No. 50593016), nucleic acid encoding a mouse IFN beta polypeptide of the nucleic acid sequence set forth in GenBank® Accession Nos. NM_010510.1 (GI No. 6754303), BC119395.1 (GI No. 111601321), or BC119397.1 (GI No. 111601034), and nucleic acid encoding a rat IFN beta polypeptide of the nucleic acid sequence set forth in GenBank® Accession No. NM_019127.1 (GI No. 9506800).

A nucleotide sequence that is a template for a positive sense transcript encoding a NIS polypeptide may be positioned downstream of nucleotide sequence encoding a Paramyxovirus (e.g., a measles virus) F polypeptide or nucleotide encoding a Paramyxovirus (e.g., a measles virus) H polypeptide. For example, a nucleotide sequence that is a template for a positive sense transcript encoding a NIS polypeptide can be positioned between the nucleotide sequence that is a template for a positive sense transcript encoding a Paramyxovirus (e.g., a measles virus) F or H polypeptide and nucleotide sequence that is a template for a positive sense transcript encoding a VSV L polypeptide. Such a position of may allow the viruses to express an amount of NIS polypeptide that (a) is effective to allow selective accumulation of iodide in infected cells, thereby allowing both imaging of viral distribution using radioiso-topes and radiotherapy targeted to infected cancer cells, and (b) is not so high as to be toxic to infected cells.

Any appropriate nucleotide sequence that is a template for a positive sense transcript encoding a NIS polypeptide can be inserted into the genome of a recombinant virus disclosed herein, for example, a recombinant virus comprising a rhabdovirus (e.g., vesiculovirus, including, without limitation, VSV) genome. For example, a nucleotide sequence that is a template for a positive sense transcript encoding a human NIS polypeptide may be inserted into the genome of a vesicular stomatitis virus. Examples of a nucleotide encoding NIS polypeptides include, without limitation, nucleotide encoding a human NIS polypeptide of the nucleotide sequence set forth in GenBank® Accession Nos. NM_000453.2 (GI No. 164663746), BC105049.1 (GI No. 85397913), or BC105047.1 (GI No. 85397519), nucleotide encoding a mouse NIS polypeptide of the nucleotide sequence set forth in GenBank® Accession Nos. NM_053248.2 (GI No. 162138896), AF380353.1 (GI No. 14290144), or AF235001.1 (GI No. 12642413), nucleotide encoding a chimpanzee NIS polypeptide of the nucleotide sequence set forth in GenBank® Accession No. XM_524154 (GI No. 114676080), nucleotide encoding a dog NIS polypeptide of the nucleotide sequence set forth in GenBank® Accession No. XM_541946 (GI No. 73986161), nucleotide encoding a cow NIS polypeptide of the nucleotide sequence set forth in GenBank® Accession No. XM_581578 (GI No. 297466916), nucleotide encoding a pig NIS polypeptide of the nucleotide sequence set forth in GenBank® Accession No. NM_214410 (GI No. 47523871), and nucleotide encoding a rat NIS polypeptide of the nucleotide sequence set forth in GenBank® Acces-sion No. NM_052983 (GI No. 158138504).

In some embodiments, a recombinant virus disclosed herein may comprise a nucleotide sequence that is a template for a positive sense transcript encoding an immune stimulatory molecule. Immune stimulatory molecules may include proteins which may participate in the induction of an immune response, proteins which may relieve inhibitory signals to the induction or effectiveness of an immune response, and/or RNA molecules (e.g., shRNA, antisense RNA, RNAi or micro-RNA) which may inhibit the expression of immune inhibitory molecules.

Examples of immune stimulatory molecules include, without limitation, GM-CSF, IL-2, IL-12, IL-15, IL-18, IL-21, IL-24, a type I interferon, interferon gamma, a type III interferon, TNF alpha, an antagonist of TGF beta, an immune checkpoint antagonist, an agonist of an immune potentiating pathway, an agonist of CD40, ICOS, GITR, 4-1-BB, OX40 or flt3, CD40 ligand (CD40L), ICOS ligand, GITR ligand, 4-1-BB ligand, OX40 ligand, and flt3 ligand.

In some embodiments, an immune stimulatory molecule encoded by a recombinant virus of the disclosure may include, e.g., C—C motif chemokine ligand 4 (CCL4), soluble lymphotoxin (sLT), interferon gamma (IFNg), interleukin 15 (IL15/15RaTM; sIL15/15Ra), adenosine deaminase 1 and 2 (hADA1; hADA2; mADA), C—C motif chemokine ligand 21 (CCL21), C—C motif chemokine ligand 19 (CCL19), C—C motif chemokine Ligand 13 (CXCL13), decoy resistant IL-18 (an engineered IL-18 variant which can bind an IL-18 receptor with increased affinity but is not neutralized by IL-18BP) (DR18), macrophage inflammatory protein-1 alpha (MIP-1 alpha) aka CCL3 (MIP1a), p14 fusion-associated small transmembrane (FAST) (P14-FAST), interferon alpha 1 (IFNa1), interferon alpha 4 (IFNa4), and/or measles P protein (measles P; MeV.P).

In some embodiments, the immune stimulatory molecule may be an immune checkpoint antagonist such as, but not limited to, antibodies, single chain antibodies and RNAi/siRNA/microRNA/antisense RNA knockdown approaches that inhibit the activity of a checkpoint inhibitor.

Agonists of immune potentiating/co-stimulatory pathways include mutant or wild type, soluble, secreted and/or membrane bound ligands, and agonistic antibodies including single chain antibodies. Related to the targeting of immune coinhibitory or immune costimulatory pathways, proteins or other molecules (agonistic or antagonistic depending on the case) targeting, e.g., CTLA-4 (antagonist), PD-1 (antagonist), PD-L1 (antagonist), LAG-3 (antagonist), TIM-3 (antagonist), VISTA (antagonist), CSF1R (antagonist), IDO (antagonist), CEACAMI (antagonist), GITR (agonist), 4-1-BB (agonist), KIR (antagonist), SLAMF7 (antagonist), OX40 (agonist), CD40 (agonist), ICOS (agonist), or CD47 (antagonist), may be used in accordance with the disclosure.

In some embodiments, the immune checkpoint antagonist may comprise a checkpoint inhibitor, for example, IL-12, CTLA-4 or CTLA4 also known as CD152 (cluster of differentiation 152). In some embodiments, the immune checkpoint antagonist may comprise a checkpoint inhibitor, for example, programmed cell death protein 1, also known as PD-1 and CD279.

In some embodiments, the immune checkpoint antagonist is an anti-CTLA4 antibody. In some embodiments, the immune checkpoint antagonist is an anti-CTLA4 antibody or an anti-PD-1 antibody.

In some embodiments, a recombinant virus of the disclosure may encode OX40L, 4-1-BBL, GITRL, ICOSL, GM-CSF and/or a wild type or modified version of CD40L. A non-limiting example of an inhibitor of a co-inhibitory pathway is a CTLA-4 inhibitor. The CTLA-4 inhibitor is generally a molecule, e.g., peptide or protein that binds to CTLA-4 and reduces or blocks signaling via CTLA-4, such as by reducing activation by B7. By reducing CTLA-4 signaling, the inhibitor reduces or removes the block of immune stimulatory pathways by CTLA-4. The CTLA-4 inhibitor may comprise an antibody or an antigen binding fragment thereof.

In some embodiments, a recombinant virus disclosed herein may comprise a nucleotide sequence that is a template for a positive sense transcript encoding an immune inhibitory molecule (e.g., an immune combat molecule). Immune combat molecules may include proteins which may participate in the suppression of an immune response, proteins which may simulate or promote inhibitory signals to the induction or effectiveness of an immune response, and/or RNA molecules (e.g., shRNA, antisense RNA, RNAi or micro-RNA) which may inhibit the expression of immune stimulatory molecules. Examples of immune combating molecules include, without limitation, vaccinia E3L dsRNA binding protein (Vaccinia E3L; Vac E3L), ubiquitin specific peptidase 18 (USP18; hUSP18; mUSP18), and measles V protein (Measles V; MeV.V).

In some embodiments, the recombinant virus of the present disclosure may further comprise a nucleotide sequence that is a template for a positive sense transcript encoding a reporter polypeptide. A non-limiting example of a reporter polypeptide that may be used in accordance with the present disclosure is luciferase. In some embodiments, the luciferase may be selected from Renilla luciferase, RLuc8 mutant Renilla luciferase, (dCpG)Luciferase, NanoLuc reporter, firefly luciferase, Gaussia luciferase (gLuc), MetLuc, Vibrio fischeri lumazine protein, Vibrio harveyi luminaze protein, inoflagellate luciferase, firefly luciferase YY5 mutant, firefly luciferase LGR mutant, firefly luciferase mutant E, and functional fragments or derivatives thereof.

In some embodiments, the reporter polypeptide is a fluorescent protein. In some embodiments, the fluorescent protein may be selected from green fluorescent protein (GFP), GFP-like fluorescent proteins, (GFP-like), enhanced green fluorescent protein (EGFP), yellow fluorescent protein (YFP), enhanced yellow fluorescent protein (EYFP), blue fluorescent protein (BFP), enhanced blue fluorescent protein (EBFP), cyan fluorescent protein (CFP), enhanced cyan fluorescent protein (ECFP), red fluorescent protein, superfolder GFP, superfolder YFP, orange fluorescent protein, red fluorescent protein, small ultrared fluorescent protein, FMN-binding fluorescent protein, dsRed, qFP611, Dronpa, TagRFP, KFP, EosFP, IrisFP, Dendra, Kaede, KikGrl, emerald fluorescent protein, Azami Green, mWasabi, TagGFP, TurboGFP, AcGFP, ZsGreen, T-Sapphire, and functional fragments or derivatives thereof.

Any appropriate method can be used to insert a nucleic acid sequence disclosed herein (e.g., a nucleotide sequence that is a template for a positive sense transcript encoding an IFN polypeptide) into the genome of a vesicular stomatitis virus.

### Host Cells and Virus Production

In one aspect, the present disclosure provides a host cell comprising a recombinant virus (e.g., a recombinant pseudotyped virus) or polynucleotide molecule described herein. A host cell can be any cell that comprises a heterologous nucleic acid. A host cell, for example, without limitation, may be a cell from any organism that is used, manipulated, modified, selected, transformed, or grown, for the production of a substance by the cell, e.g., the expression by the cell of, an RNA or DNA sequence, a gene, a protein, or an enzyme.

In some embodiments, the host cell may be selected based on the vector backbone. In some embodiments, a cosmid or plasmid or may be introduced into a prokaryote host cell for replication of several types of vectors. Bacterial cells including E. coli and B. subtilis may be used as host cells for vector replication and/or expression or for phage viruses. Eukaryotic cells that can be used as host cells include, but are not limited to mammals, insects and yeast. Non-limiting examples of mammalian eukaryotic host cells are PC12, NIH3T3, HeLa, COS, Jurkat, 293, CHO, and Saos.

Packaging cells useful for production of the recombinant viruses, e.g., recombinant pseudotyped viruses, described herein include, e.g., animal cells permissive for the virus, or cells modified to be permissive for the virus; or the packaging cell construct, for example, with the use of a transformation agent such as calcium phosphate. Non-limiting examples of packaging cell lines useful for producing recombinant viruses described herein include, e.g., human embryonic kidney 293 (HEK-293) cells (e.g., American Type Culture Collection [ATCC] No. CRL-1573), HEK-293 cells that contain the SV40 Large T-antigen (HEK-293T or 293T), HEK293T/17 cells, human sarcoma cell line HT-1080 (CCL-121), lymphoblast-like cell line Raj i (CCL-86), glioblastoma-astrocytoma epithelial-like cell line U87-MG (HTB-14), T-lymphoma cell line HuT78 (TIB-161), NIH/3T3 cells, Chinese Hamster Ovary cells (CHO) (e.g., ATCC Nos. CRL9618, CCL61, CRL9096), HeLa cells (e.g., ATCC No. CCL-2), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RATI cells, mouse L cells (ATCC No. CCLI.3), HLHepG2 cells, CAP cells, CAP-T cells, and the like.

Further non-limiting examples of packaging cells and/or systems that may be useful for the production of recombinant viruses described herein include, for example, L929 cells, the FLY viral packaging cell system outlined in Cosset et al (1995) J Virol 69, 7430-7436, NS0 (murine myeloma) cells, human amniocytic cells (e.g., CAP, CAP-T), yeast cells (including, but not limited to, S. cerevisiae, Pichia pastoris), plant cells (including, but not limited to, Tobacco NT1, BY-2), insect cells (including but not limited to SF9, S2, SF21, Tni (e.g. High 5)) or bacterial cells (including, but not limited to, E. coli).

Additional packaging cells and systems, packaging techniques and vectors for packaging the nucleic acid genome into the recombinant virus may include method steps comprising, e.g., construction of structural protein expression cassettes comprising plasmids for vector-inducible expression of virus structural proteins, and incorporation of any additional elements by polymerase chain reaction (PCR) amplification or by using synthetic oligonucleotides. By way of a non-limiting example, for selection, screening, and/or characterization of packaging cell lines, cells transfected with expression cassette constructs may be selected with, e.g., G418 or hygromycin. Pooled foci of drug-resistant cells may be cloned by limiting dilution, and individual clones may be screened for packaging activity, e.g., by transfection with a vector using, e.g., Lipofection or electroporation. Those clones with the highest levels of activity may be expanded for further use. Northern and western blot analysis of vector-specific or structural protein-specific RNA and proteins expressed in packaging cells may be performed, for example, to confirm presence or absence of such vector-specific or structural protein-specific RNA and proteins and/or determine levels of the vector-specific or structural protein-specific RNA and proteins. The titer of replication-incompetent vector particles in clarified packaging cell line culture supernatants may be determined, e.g., by infection of naïve monolayers with serial dilutions, X-gal staining and counting the total number of stained cells per well at the appropriate dilution. Vector titer may be designated as infectious units (IU)/ml. Contaminating replication-competent virus in culture supernatants may detected by standard plaque assay (plaque-forming units or PFU/ml) and by serial undiluted passages in naïve cells. Methods of packaging include using packaging cells that permanently express the viral components, or by transiently transfecting cells with plasmids.

In one aspect, the current disclosure provides cells for production of a recombinant virus, e.g., a recombinant pseudotyped virus, described herein. Example cells include, but are not limited to, any cell in which a virus such as a rhabdovirus described herein grows, e.g., mammalian cells and some insect (e.g., Drosophila) cells. A vast number of primary cells and cell lines commonly known in the art can be used as host cells. By way of example, a cell line that can used to produce a recombinant virus disclosed herein includes but is not limited to BHK (baby hamster kidney) cells, CHO (Chinese hamster ovary) cells, HeLA (human) cells, mouse L cells, Vero (monkey) cells (including Vero-αHis cells), Vero-Ace-2 cells, Vero-TRMPSS2 cells, Vero-E6 cells, ESK-4, PK-15, EMSK cells, MDCK (Madin-Darby canine kidney) cells, MDBK (Madin-Darby bovine kidney) cells, 293 (human) cells, Hep-2 cells, primary chick embryo fibroblasts, primary chick embryo fibroblasts, quasi-primary continuous cell lines (e.g. AGMK-African green monkey kidney cells), human diploid primary cell lines (e.g. WI-38 and MRC5 cells), and Monkey Diploid Cell Line (e.g. FRhL-Fetal Rhesus Lung cells).

In a related aspect, described herein is a method of producing a recombinant virus disclosed herein. Such a method may comprise culturing a packaging cell in conditions sufficient to produce a plurality of recombinant viruses. In some embodiments, the method further comprises collecting the recombinant viruses. In one specific embodiment, the collecting step comprises one or more of the following steps: clearing cell debris, treating the supernatant containing recombinant viruses with DNase I and MgCl₂, concentrating the recombinant viruses, and purifying the recombinant viruses.

In the methods described herein, plasmids/vectors used for recombinant virus production can be introduced into the packaging cells using, e.g., electroporation (using for example Multiporator, Genepulser, MaxCyte Transfection Systems), PEI, Ca2+-mediated transfection or via liposomes (for example: “Lipofectamine”), non-liposomal compounds (for example: “Fugene” or nucleofection) into cells.

As an example, recombinant VSV particles described herein can be produced using, e.g., by providing in an appropriate host cell: (a) DNA that can be transcribed to encode VSV antigenomic (+) RNA (complementary to the VSV genome), (b) a recombinant source of VSV nucleoprotein (N) protein, (c) a recombinant source of VSV phosphoprotein (P) protein, (d) a recombinant source of VSV large protein (L), and (e) foreign DNA; under conditions such that the DNA is transcribed to produce the antigenomic RNA, and a VSV is produced that contains genomic RNA complementary to the antigenomic RNA produced and foreign RNA, which is not naturally a part of the VSV genome, from the DNA. Methods and compositions useful for generating recombinant (e.g., VSV) particles may be found, for example, in U.S. Pat. Nos. 7,153,510; 9,861,668; 8,012,489; 9,630,996; 8,287,878; 9,248,178; U.S. Patent Publication Nos. 2014/0271564; 2012/0121650; Fukishi et al., J. Gen. Virol., 2005, 86:2269-2274, each of which are incorporated by reference herein in their entirety.

Any DNA that can be transcribed to produce VSV antigenomic (+) RNA (complementary to the VSV genome) can be used for the construction of a recombinant DNA containing foreign DNA encoding a heterologous (foreign) protein or peptide, for use in producing the recombinant VSV particles described herein. In certain embodiments, the DNA that can be transcribed to encode VSV antigenomic (+) RNA comprises at least genes for the VSV N protein, the VSV P protein, and the VSV L protein. In certain embodiments, the DNA that can be transcribed to encode VSV antigenomic (+) RNA comprises at least genes for the VSV N protein, the VSV P protein, the VSV L protein, and the foreign protein or peptide (e.g., a fusogenic molecule). In certain embodiments, DNA that can be transcribed to encode VSV antigenomic (+) RNA can further encode the VSV matrix (M) protein and/or G glycoprotein.

As an example, DNA that can be transcribed to produce VSV antigenomic (+) RNA (such DNA being referred to herein as “VSV (−) DNA”) is available in the art and/or can be obtained by standard methods. VSV (−) DNA for any serotype or strain known in the art, e.g., the New Jersey or Indiana serotypes of VSV, can be used. The complete nucleotide and deduced protein sequence of the VSV genome is known, and is available as Genbank VSVCG, Accession No. J02428; NCBI Seq ID 335873; An example of the complete sequence of the VSV(−) DNA that is contained in plasmid pVSVFL(+) is deposited with the American Type Culture Collection (ATCC) and assigned Accession No. 97134. In some embodiments, the nucleotide sequence of plasmid pVSVFL(+) comprises the nucleotide sequence of SEQ ID NO: 144.

VSV (−) DNA, if not already available, can be prepared as follows: VSV genomic RNA can be purified from virus preparations, and reverse transcription with long distance polymerase chain reaction can be used to generate the VSV (−) DNA. Alternatively, after purification of genomic RNA, VSV mRNA can be synthesized in vitro, and cDNA can be prepared, followed by insertion into cloning vectors. Individual cDNA clones of VSV RNA can be joined by use of small DNA fragments covering the gene junctions, generated by use of reverse transcription and polymerase chain reaction (RT-PCR) (from VSV genomic RNA).

In certain embodiments, one or more, usually unique, restriction sites (e.g., in a polylinker) are introduced into the VSV (−) DNA, in intergenic regions, or 5′ of the sequence complementary to the 3′ end of the VSV genome, or 3′ of the sequence complementary to the 5′ end of the VSV genome, to facilitate insertion of the foreign DNA.

In certain embodiments, the recombinant virus is constructed to have a promoter operatively linked thereto. The promoter should be capable of initiating transcription of the (−) DNA in an animal or insect cell in which it is desired to produce the recombinant virus. Promoters which may be used include, but are not limited to, the SV40 early promoter region, the promoter contained in the 3′ long terminal repeat of Rous sarcoma virus, the herpes thymidine kinase promoter, the regulatory sequences of the metallothionein gene; heat shock promoters (e.g., hsp70 for use in Drosophila S2 cells); the ADC (alcohol dehydrogenase) promoter, PGK (phosphoglycerol kinase) promoter, alkaline phosphatase promoter, and the following animal transcriptional control regions, which exhibit tissue specificity and can be used in transgenic animals: elastase I gene control region which is active in pancreatic acinar cells; insulin gene control region which is active in pancreatic beta cells; immunoglobulin gene control region which is active in lymphoid cells; mouse mammary tumor virus control region which is active in testicular, breast, lymphoid, and mast cells; albumin gene control region which is active in liver; alpha-fetoprotein gene control region which is active in liver; alpha 1-antitrypsin gene control region which is active in the liver; beta-globin gene control region which is active in myeloid cells, myelin basic protein gene control region which is active in oligodendrocyte cells in the brain; and myosin light chain-2 gene control region which is active in skeletal muscle. Preferably, the promoter is an RNA polymerase promoter, preferably a bacteriophage or viral or insect RNA polymerase promoter, including but not limited to the promoters for T7 RNA polymerase, SP6 RNA polymerase, and T3 RNA polymerase. If an RNA polymerase promoter is used in which the RNA polymerase is not endogenously produced by the host cell in which it is desired to produce the recombinant VSV, a recombinant source of the RNA polymerase must also be provided in the host cell.

The VSV (−) DNA can be operably linked to a promoter before or after insertion of foreign DNA. In certain embodiments, a transcriptional terminator is situated downstream of the VSV (−) DNA.

In another embodiment, a DNA sequence that can be transcribed to produce a ribozyme sequence is situated at the immediate 3′ end of the VSV (−) DNA, prior to the transcriptional termination signal, so that upon transcription a self-cleaving ribozyme sequence is produced at the 3′ end of the antigenomic RNA, which ribozyme sequence will autolytically cleave (after a U) this fusion transcript to release the exact 3′ end of the VSV antigenomic (+) RNA. Any ribozyme sequence may be used if the correct sequence is recognized and cleaved. In a preferred aspect, hepatitis delta virus (HDV) ribozyme is used.

An example of a VSV (−) DNA for use, for insertion of foreign DNA, can thus comprise (in 5′ to 3′ order) the following operably linked components: the T7 RNA polymerase promoter, VSV (−) DNA, a DNA sequence that is transcribed to produce an HDV ribozyme sequence (immediately downstream of the VSV (−) DNA), and a T7 RNA polymerase transcription termination site.

Examples of plasmids that can be used in the present disclosure are pVSVFL(+) or pVSVSS1.

In certain embodiments, foreign DNA is inserted into an intergenic region, or a portion of the VSV (−) DNA that is transcribed to form the noncoding region of a viral mRNA. In certain embodiments, the foreign DNA is inserted into a coding region of the VSV genome that is non-essential to the virus's development, growth and/or functions. In certain embodiments, the VSV G gene is disrupted. In certain embodiments, the foreign DNA insertion does not disrupt the G gene or VSV G protein function. Any method known to one skilled in the art maybe used for large scale production of recombinant viruses, packaging cells and vector constructs described herein. For example, master and working seed stocks may be prepared under good manufacturing practice (GMP) conditions in qualified primary CEFs or by other methods. Packaging cells may be plated on large surface area flasks, grown to near confluence and recombinant viruses purified. Cells may be harvested, and recombinant viruses released into the culture media isolated and purified, or intracellular recombinant viruses released by mechanical disruption (cell debris can be removed by large-pore depth filtration and cell, e.g., host cell, DNA digested with endonuclease). Viruses may be subsequently purified and concentrated by tangential-flow filtration, followed by diafiltration. The resulting concentrated bulk may be formulated by dilution with a buffer containing stabilizers, filled into vials, and lyophilized. Compositions and formulations may be stored for later use. For use, lyophilized recombinant viruses may be reconstituted by addition of diluent.

### Pharmaceutical Compositions

Also disclosed herein are compositions including pharmaceutical compositions comprising the recombinant polynucleotides, recombinant viruses, and/or cell-derived nanovesicles described herein and a pharmaceutically acceptable carrier and/or excipient. In addition, disclosed herein are pharmaceutical dosage forms comprising the recombinant polynucleotides, recombinant viruses, and/or cell-derived nanovesicles described herein.

Pharmaceutical compositions based on the recombinant polynucleotides, recombinant viruses, and/or cell-derived nanovesicles disclosed herein can be formulated in any conventional manner using one or more physiologically acceptable carriers and/or excipients. The recombinant polynucleotides, recombinant viruses, and/or cell-derived nanovesicles may be formulated for administration by, for example, injection, inhalation or insulation (either through the mouth or the nose) or by oral, buccal, parenteral or rectal administration, or by administration directly to a tumor.

The pharmaceutical compositions can be formulated for a variety of modes of administration, including systemic, topical, or localized administration. Techniques and formulations can be found in, for example, Remington's Pharmaceutical Sciences, Meade Publishing Co., Easton, Pa. For systemic administration, injection is preferred, including intramuscular, intravenous, intraperitoneal, and subcutaneous. For the purposes of injection, the pharmaceutical compositions can be formulated in liquid solutions, preferably in physiologically compatible buffers, such as Hank's solution or Ringer's solution. In addition, the pharmaceutical compositions may be formulated in solid form and redissolved or suspended immediately prior to use. Lyophilized forms of the pharmaceutical composition are also suitable.

For oral administration, the pharmaceutical compositions may take the form of, for example, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g. potato starch or sodium starch glycolate); or wetting agents (e.g., sodium lauryl sulfate). The tablets can also be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., ationd oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate.

The pharmaceutical compositions can be formulated for parenteral administration by injection, e.g., by bolus injection or continuous infusion. Formulations for injection can be presented in a unit dosage form, e.g., in ampoules or in multi-dose containers, with an optionally added preservative. The pharmaceutical compositions can further be formulated as suspensions, solutions, or emulsions in oily or aqueous vehicles, and may contain other agents including suspending, stabilizing and/or dispersing agents.

Additionally, the pharmaceutical compositions can also be formulated as a depot preparation. These long-acting formulations can be administered by implantation (e.g., subcutaneously or intramuscularly) or by intramuscular injection. Thus, for example, the compounds may be formulated with suitable polymeric or hydrophobic materials (e.g., as an emulsion in an acceptable oil) or ion exchange resins, or as sparingly soluble derivatives, for example, as a sparingly soluble salt. Other suitable delivery systems include microspheres, which offer the possibility of local noninvasive delivery of drugs over an extended period of time. This technology can include microspheres having a precapillary size, which can be injected via a coronary catheter into any selected part of an organ without causing inflammation or ischemia. The administered therapeutic is then slowly released from the microspheres and absorbed by the surrounding cells present in the selected tissue.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, bile salts, and fusidic acid derivatives. In addition, detergents may be used to facilitate permeation. Transmucosal administration can occur using nasal sprays or suppositories. For topical administration, the recombinant polynucleotides, recombinant viruses, and/or cell-derived nanovesicles described herein can be formulated into ointments, salves, gels, or creams as generally known in the art. A wash solution can also be used locally to treat an injury or inflammation in order to accelerate healing.

Pharmaceutical forms suitable for injectable use can include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid. It must be stable under the conditions of manufacture and certain storage parameters (e.g., refrigeration and freezing) and must be preserved against the contaminating action of microorganisms, such as bacteria and fungi.

If formulations disclosed herein are used as a therapeutic to boost an immune response in a subject, a therapeutic agent can be formulated into a composition in a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

A carrier can also be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, using a coating, such as lecithin, by the maintenance of the required particle size in the case of dispersion and by the use of surfactants. The prevention of the action of microorganisms can be brought about by various antibacterial and antifungal agents known in the art. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Compositions as disclosed herein can also include adjuvants such as aluminum salts and other mineral adjuvants, tensoactive agents, bacterial derivatives, vehicles, and cytokines. Adjuvants can also have immunomodulating properties. For example, adjuvants can stimulate Th1 or Th2 immunity. Compositions and methods as disclosed herein can also include adjuvant therapy.

Sterile injectable solutions can be prepared by incorporating the active compounds or constructs in the required amount in the appropriate solvent with various of the other ingredients enumerated above, as required, followed by filtered sterilization.

Upon formulation, solutions can be administered in a manner compatible with the dosage formulation and in such amount as is therapeutically effective. The formulations are easily administered in a variety of dosage forms, such as the type of injectable solutions described above, but slow-release capsules or microparticles and microspheres and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intratumorally, intramuscular, subcutaneous and intraperitoneal administration. In this context, sterile aqueous media that can be employed will be known to those of skill in the art considering the present disclosure. For example, one dosage could be dissolved in 1 ml of isotonic NaCl solution and either added to 1000 ml of hypodermoclysis fluid or injected at the proposed site of infusion.

The person responsible for administration will, in any event, determine the appropriate dose for the individual subject. For example, a subject may be administered recombinant polynucleotides, recombinant viruses, and/or cell-derived nanovesicles described herein on a daily or weekly basis for a time period or on a monthly, bi-yearly or yearly basis depending on need or exposure to a pathogenic organism or to a condition in the subject (e.g., cancer).

In addition to the compounds formulated for parenteral administration, such as intravenous, intratumorally, intradermal or intramuscular injection, other pharmaceutically acceptable forms include, e.g., tablets or other solids for oral administration; liposomal formulations; time release capsules; biodegradable and any other form currently used.

One may also use intranasal or inhalable solutions or sprays, aerosols, or inhalants. Nasal solutions can be aqueous solutions designed to be administered to the nasal passages in drops or sprays. Nasal solutions can be prepared so that they are similar in many respects to nasal secretions. Thus, the aqueous nasal solutions usually are isotonic and slightly buffered to maintain a pH of 5.5 to 7.5. In addition, antimicrobial preservatives, similar to those used in ophthalmic preparations, and appropriate drug stabilizers, if required, may be included in the formulation. Various commercial nasal preparations are known and can include, for example, antibiotics and antihistamines and are used for asthma prophylaxis.

Oral formulations can include excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders. In certain defined embodiments, oral pharmaceutical compositions will include an inert diluent or assimilable edible carrier, or they may be enclosed in hard- or soft-shell gelatin capsule, or they may be compressed into tablets, or they may be incorporated directly with the food of the diet. For oral therapeutic administration, the active compounds may be incorporated with excipients and used in the form of ingestible tablets, buccal tablets, troches, capsules, elixirs, suspensions, syrups, wafers, and the like.

The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup of elixir may contain the active compounds sucrose as a sweetening agent methyl and propylparabens as preservatives, a dye and flavoring, such as cherry or orange flavor.

Certain additional agents used in the combination therapies can be formulated and administered by any means known in the art.

Dose ranges and frequency of administration can vary depending on the nature of the recombinant polynucleotides, recombinant viruses, and/or cell-derived nanovesicles and the medical condition as well as parameters of a specific patient and the route of administration used. In some embodiments, recombinant viral compositions can be administered to a subject at a dose ranging from about 1×10⁵ plaque forming units (pfu) to about 1×10¹⁵ pfu, depending on mode of administration, the route of administration, the nature of the disease and condition of the subject. In some cases, the recombinant viral compositions can be administered at a dose ranging from about 1×10⁸ pfu to about 1×10¹⁵ pfu, or from about 1×10¹⁰ pfu to about 1×10¹⁵ pfu, or from about 1×10⁸ pfu to about 1×10¹² pfu. A more accurate dose can also depend on the subject in which it is being administered. For example, a lower dose may be required if the subject is juvenile, and a higher dose may be required if the subject is an adult human subject. In certain embodiments, a more accurate dose can depend on the weight of the subject. In certain embodiments, for example, a juvenile human subject can receive from about 1×10⁸ pfu to about 1×10¹⁰ pfu, while an adult human subject can receive a dose from about 1×10¹⁰ pfu to about 1×10¹² pfu.

Compositions disclosed herein may be administered by any means known in the art. For example, compositions may include administration to a subject intravenously, intratumorally, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intrathecally, subcutaneously, subconjunctival, intravesicularlly, mucosally, intrapericardially, intraumbilically, intraocularly, orally, locally, by inhalation, by injection, by infusion, by continuous infusion, by localized perfusion, via a catheter, via a lavage, in a cream, or in a lipid composition. In some embodiments, the compositions disclosed herein are administered intravenously or intratumorally.

Recombinant polynucleotides, recombinant viruses, and/or cell-derived nanovesicles of the present disclosure, or pharmaceutical compositions comprising such recombinant polynucleotides, recombinant viruses, and/or cell-derived nanovesicles, may be directly administered, for example, via direct injection. By way of a non-limiting example, a virus may be injected directly into a group of cancer cells or into a tumor. In some embodiments, the tumor may be palpable through the skin of a subject. Ultrasound guidance may also be useful in the practice of such a method. In some embodiments, direct administration of a recombinant polynucleotides, recombinant viruses, and/or cell-derived nanovesicles disclosed herein may be achieved via a catheter line or other medical access device and may be used in conjunction with an imaging system such as, to localize a group of cancer cells. By such methods, an implantable dosing device typically is placed in proximity to a group of cancer cells using a guidewire inserted into the medical access device. An effective dose of a recombinant polynucleotides, recombinant viruses, and/or cell-derived nanovesicles disclosed herein may be directly administered to a tumor or group of cancer cells that is visible within an exposed surgical field.

The course of therapy with a recombinant polynucleotide, recombinant viruses, and/or cell-derived nanovesicles provided herein may be monitored by evaluating changes in clinical symptoms or by direct monitoring of the number of cancer cells or size of a tumor. For a solid tumor, the effectiveness of virus treatment can be assessed by measuring the size or weight of the tumor before and after treatment. Tumor size can be measured either by using imaging techniques (e.g., X-ray, magnetic resonance imaging, or computerized tomography), by evaluation of non-imaging optical data (e.g, spectral data), or directly (e.g, by use of calipers). For a group of cancer cells (e.g., leukemia cells), the effectiveness of viral treatment can be determined by measuring the absolute number of cancer cells in the circulation of a patient before and after treatment. The effectiveness of viral treatment also can be assessed by monitoring the levels of a cancer specific antigen.

Further embodiments disclosed herein can concern kits for use with methods and compositions. Kits can also include a suitable container, for example, vials, tubes, mini- or microfuge tubes, test tube, flask, bottle, syringe, or other container. Where an additional component or agent is provided, the kit can contain one or more additional containers into which this agent or component may be placed. Kits herein will also typically include a means for containing the recombinant polynucleotides, recombinant viruses, and/or cell-derived nanovesicles and any other reagent containers in close confinement for commercial sale. Such containers may include injection or blow-molded plastic containers into which the desired vials are retained. Optionally, one or more additional active agents such as, e.g., anti-inflammatory agents, anti-viral agents, anti-fungal, anti-bacterial agents, or anti-tumor agents may be needed for compositions described.

### Methods of Use

The recombinant polynucleotide, recombinant virus such as but not limited to a recombinant rhabdovirus (e.g., a recombinant VSV) or recombinant pseudotyped virus described herein, and/or cell-derived nanovesicle compositions, e.g., pharmaceutical compositions comprising recombinant polynucleotides, recombinant viruses, and/or cell-derived nanovesicles described herein, and a carrier and/or excipient, may be used for various therapeutic applications (in vivo and ex vivo) and as research tools. In one aspect, described herein is a method for treating a disease (e.g., cancer) in a subject in need thereof, the method comprising administering to the subject a therapeutically effective amount of the recombinant virus, and/or cell-derived nanovesicle disclosed herein and/or the pharmaceutical composition thereof.

In certain aspects, the present disclosure provides methods for treating cancer (e.g, to reduce tumor size, inhibit tumor growth, or reduce the number of viable tumor cells). For example, a recombinant virus, and/or cell-derived nanovesicle, or composition thereof, provided herein may be administered to a subject having cancer to reduce tumor size, inhibit cancer cell or tumor growth, reduce the number of viable cancer cells within the subject, and/or to induce host immunogeneic responses against a tumor.

In certain aspects, the present disclosure provides methods for inducing cancer regression in a subject in need thereof. The method may comprise administering to a subject an effective amount of the recombinant virus and/or cell-derived nanovesicle or composition of the present disclosure.

In some embodiments, the present disclosure provides, among other things, a method of treating a cancer in a subject in need thereof comprising administering to the subject a therapeutically effective amount of a recombinant virus, and/or cell-derived nanovesicle or a composition thereof, and a carrier and/or excipient. In some embodiments, the method does not include pre-treatment with LDL/VLDL-lowering medications. In some embodiments, the method further comprises pre-treatment with LDL/VLDL-lowering medications.

In some embodiments, the administration of, e.g., a recombinant virus or pharmaceutical composition thereof, disclosed herein to the subject is under conditions where the recombinant virus infects cancer cells of a subject to form infected cancer cells.

In certain embodiments, the recombinant polynucleotide, recombinant virus, and/or cell-derived nanovesicle or the pharmaceutical composition thereof may be administered, for example, intravenously, subcutaneously, intramuscularly, transdermally, intranasally, orally, or mucosally.

In some embodiments, the recombinant polynucleotide, recombinant virus, and/or cell-derived nanovesicle or pharmaceutical composition thereof provided herein may be administered to a subject in need thereof by, for example, direct injection into a group of cancer cells (e.g, a tumor) or via intravenous delivery. In some embodiments, a recombinant virus and/or pharmaceutical thereof disclosed herein may be used to treat different types of cancer such as, but not limited to, myeloma (e.g, multiple myeloma), melanoma, glioma, lymphoma, mesothelioma, and cancers of the lung, brain, stomach, colon, rectum, kidney, prostate, ovary, breast, pancreas, liver, and head and neck.

In some embodiments, a recombinant polynucleotide, recombinant virus, and/or cell-derived nanovesicle or composition thereof is administered via systemic delivery. In some embodiments, the recombinant virus or composition is administered via parenteral delivery. Non-limiting examples of parenteral delivery include subcutaneous, intraperitoneal, intradermal, intramuscular, or intravenous delivery. In some embodiments, the parental delivery is intravenous delivery.

Non-limiting examples of cancers treatable by the methods described herein include, for example, carcinomas, lymphomas, sarcomas, blastomas, and leukemias. Non-limiting specific examples, include, for example, breast cancer, pancreatic cancer, liver cancer, lung cancer, prostate cancer, colon cancer, renal cancer, bladder cancer, head and neck carcinoma, thyroid carcinoma, soft tissue sarcoma, ovarian cancer, primary or metastatic melanoma, squamous cell carcinoma, basal cell carcinoma, brain cancers of all histopathologic types, angiosarcoma, hemangiosarcoma, bone sarcoma, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, testicular cancer, uterine cancer, cervical cancer, gastrointestinal cancer, mesothelioma, Ewing's tumor, leiomyosarcoma, Ewing's sarcoma, rhabdomyosarcoma, carcinoma of unknown primary (CUP), squamous cell carcinoma, basal cell carcinoma, adenocarcinoma, sweat gland carcinoma, sebaceous gland carcinoma, papillary carcinoma, Waldenstroom's macroglobulinemia, papillary adenocarcinomas, cystadenocarcinoma, bronchogenic carcinoma, bile duct carcinoma, choriocarcinoma, seminoma, embryonal carcinoma, Wilms' tumor, lung carcinoma, epithelial carcinoma, cervical cancer, testicular tumor, glioma, glioblastoma, astrocytoma, medulloblastoma, craniopharyngioma, ependymoma, pinealoma, hemangioblastoma, acoustic neuroma, oligodendroglioma, meningioma, retinoblastoma, leukemia, neuroblastoma, small cell lung carcinoma, bladder carcinoma, lymphoma, multiple myeloma, medullary carcinoma, B cell lymphoma, T cell lymphoma, NK cell lymphoma, large granular lymphocytic lymphoma or leukemia, gamma-delta T cell lymphoma or gamma-delta T cell leukemia, mantle cell lymphoma, myeloma, leukemia, chronic myeloid leukemia, acute myeloid leukemia, chronic lymphocytic leukemia, acute lymphocytic leukemia, hairy cell leukemia, hematopoietic neoplasias, thymoma, sarcoma, non-Hodgkin's lymphoma, Hodgkin's lymphoma, Epstein-Barr virus (EBV) induced malignancies of all types including but not limited to EBV-associated Hodgkin's and non-Hodgkin's lymphoma, all forms of post-transplant lymphomas including post-transplant lymphoproliferative disorder (PTLD), uterine cancer, renal cell carcinoma, hepatoma, hepatoblastoma, Cancers that may treated by methods and compositions described herein include, but are not limited to, cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; sertoli cell carcinoma; Leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; Mullerian mixed tumor; nephroblastoma; hepatoblastoma; carcinosarcoma; mesenchymoma, malignant; Brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; Kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some embodiments, the cancers treatable by the methods described herein may be colon cancer, lung cancer, prostate cancer, ovarian cancer, hepatocellular carcinoma, pancreatic cancer, kidney cancer, melanoma, brain cancer, lymphoma, myeloma, lymphocytic leukemia, myelogenous leukemia, and/or breast cancer.

In some embodiments, the compositions and methods disclosed herein are useful to enhance the efficacy of vaccines directed to tumors or infections. Thus, the compositions and methods described herein can be administered to a subject either simultaneously with or before (e.g., 1-30 days before) a reagent (including but not limited to small molecules, antibodies, or cellular reagents) that acts to elicit an immune response (e.g., to treat cancer or an infection) is administered to the subject.

In some embodiments, the compositions and methods described herein may useful to induce an immune response in a subject in need thereof. For example, the method can comprise administering to the subject an effective amount of a recombinant virus, e.g., a recombinant pseudotyped virus, and/or a cell-derived nanovesicle, and/or a of composition thereof, described herein.

In some embodiments, the compositions and methods described herein may useful for decreasing susceptibility to serum neutralization of a recombinant pseudotyped virus and/or cell-derived nanovesicle in a subject in need thereof. For example, the method can comprise administering to the subject a pseudotyped virus and/or cell-derived nanovesicle described herein, or a composition comprising the pseudotyped virus and/or cell-derived nanovesicle and a carrier and/or excipient.

In certain some, the compositions and methods described herein may be useful for enhancing resistance to low-density lipoprotein (LDL)- and/or very-low-density lipoprotein (VLDL)-mediated neutralization of a recombinant pseudotyped virus and/or cell-derived nanovesicle in a subject in need thereof. For example, the method can comprise administering to the subject a recombinant pseudotyped virus and/or cell-derived nanovesicle described herein or a composition comprising the recombinant pseudotyped virus and/or cell-derived nanovesicle and a carrier and/or excipient.

In some embodiments, the present disclosure provides a method for delivering a molecular cargo to a cell within a subject in need thereof. For example, the method can comprise administering to the subject an effective amount of a recombinant virus described herein, e.g., a recombinant pseudotyped virus, or cell-derived nanovesicle, described herein, or a composition comprising the recombinant virus, e.g., the recombinant pseudotyped virus, or cell-derived nanovesicle and a carrier and/or excipient. In some embodiments, a recombinant fusogenic protein within the recombinant virus, e.g., the recombinant pseudotyped virus, or cell-derived nanovesicle, can comprise a targeting molecule described herein which targets a cell. The fusogenic protein can comprise any of various fusogenic proteins described herein.

It is contemplated that when used to treat various diseases, e.g., cancer, the compositions and methods disclosed herein can be combined with other therapeutic agents suitable for the same or similar diseases, for example, a second anti-cancer treatment. Also, two or more embodiments described herein may be also co-administered to generate additive or synergistic effects. When co-administered with a second therapeutic agent, the embodiment described herein, and the second therapeutic agent may be administered simultaneously or sequentially (in any order). Suitable therapeutically effective dosages for each agent may be lowered due to the additive action or synergy.

As a non-limiting example, the methods described herein can be combined with other therapies that block inflammation (e.g., via blockage of IL1, INFα/β, IL6, TNF, IL13, IL23, etc.).

In some embodiments, the methods described herein can further comprise administering to the subject a therapeutically effective amount of a second anti-cancer treatment. Non-limiting examples of a second anti-cancer treatment include an agent targeting an immune co-inhibitory or immune co-stimulatory pathway, radiation, chemotherapy, an agent that targets a specific genetic mutation which occurs in tumors, an agent intended to induce an immune response to one or more tumor antigen(s) or neoantigen(s), a cellular product of T cells or NK cells, an agent intended to stimulate the STING, cGAS, TLR or other innate immune response and/or inflammatory pathway, or a second virus, or any combinations thereof.

The compositions and methods described herein can be also administered in combination with an anti-tumor antibody or an antibody directed at a pathogenic antigen or allergen.

The compositions and methods described herein can be combined with immunomodulatory treatments such as, e.g., therapeutic vaccines (including but not limited to GVAX, DC-based vaccines, etc.), checkpoint inhibitors (including but not limited to agents that block CTLA4, PD1, LAG3, TIM3, etc.) or activators (including but not limited to agents that enhance 41BB, OX40, etc.). The inhibitory treatments described herein can be also combined with other treatments that possess the ability to modulate immune cell function or stability.

Therapeutic methods described herein can be combined with additional immunotherapies and therapies. For example, when used for treating cancer, the recombinant viruses or pharmaceutical compositions comprising such viruses described herein may be used in combination with conventional cancer therapies, such as, e.g., surgery, radiotherapy, chemotherapy or combinations thereof, depending on type of the tumor, patient condition, other health issues, and a variety of factors. In certain aspects, other therapeutic agents useful for combination cancer therapy with the inhibitors described herein include anti-angiogenic agents. Many anti-angiogenic agents have been identified and are known in the art, including, e.g., TNP-470, platelet factor 4, thrombospondin-1, tissue inhibitors of metalloproteases (TIMP1 and TIMP2), prolactin (16-Kd fragment), angiostatin (38-Kd fragment of plasminogen), endostatin, bFGF soluble receptor, transforming growth factor beta, interferon alpha, soluble KDR and FLT-1 receptors, placental proliferin-related protein, as well as those listed by Carmeliet and Jain (2000). In some embodiments, the inhibitors described herein can be used in combination with a VEGF antagonist or a VEGF receptor antagonist such as anti-VEGF antibodies, VEGF variants, soluble VEGF receptor fragments, aptamers capable of blocking VEGF or VEGFR, neutralizing anti-VEGFR antibodies, inhibitors of VEGFR tyrosine kinases and any combinations thereof (e.g., anti-hVEGF antibody A4.6.1, bevacizumab or ranibizumab).

In some embodiments, the therapeutic methods disclosed herein may comprise administering to a subject an effective amount of a second anti-cancer treatment. In some embodiments, the second anti-cancer treatment may be an agent targeting an immune co-inhibitory or immune co-stimulatory pathway, radiation, chemotherapy, an agent that targets a specific genetic mutation which occurs in tumors, an agent intended to induce an immune response to one or more tumor antigen(s) or neoantigen(s), a cellular product of T cells or NK cells, an agent intended to stimulate the STING, cGAS, TLR or other innate immune response and/or inflammatory pathway, a second virus, and any combinations thereof.

Non-limiting examples of chemotherapeutic compounds which can be used in combination treatments include, for example, aminoglutethimide, amsacrine, anastrozole, asparaginase, bcg, bicalutamide, bleomycin, buserelin, busulfan, campothecin, capecitabine, carboplatin, carmustine, chlorambucil, cisplatin, cladribine, clodronate, colchicine, cyclophosphamide, cyproterone, cytarabine, dacarbazine, dactinomycin, daunorubicin, dienestrol, diethylstilbestrol, docetaxel, doxorubicin, epirubicin, estradiol, estramnustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozocin, suramin, tamoxifen, temozolomide, teniposide, testosterone, thioguanine, thiotepa, titanocene dichloride, topotecan, trastuzumab, tretinoin, vinblastine, vincristine, vindesine, and vinorelbine.

These chemotherapeutic compounds may be categorized by their mechanism of action into, for example, following groups: anti-metabolites/anti-cancer agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethyhnelamineoxaliplatin, iphosphamide, melphalan, merchlorehtamine, mitomycin, mitoxantrone, nitrosourea, plicamycin, procarbazine, taxol, taxotere, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (e.g., TNP-470, genistein, bevacizumab) and growth factor inhibitors (e.g., fibroblast growth factor (FGF) inhibitors); angiotensin receptor blocker; nitric oxide donors; anti-sense oligonucleotides; antibodies (trastuzumab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers and caspase activators; and chromatin disruptors.

In some embodiments the therapeutic methods disclosed herein may further comprise administering to the subject an effective amount of one or more immune checkpoint inhibitors. In some embodiments, the one or more immune checkpoint inhibitors comprise anti-CTLA4 antibodies and/or anti-PD-1 antibodies. In some embodiments the one or more immune checkpoint inhibitors may include anti-PD-L1 antibodies and/or PD-L2 antibodies. In some embodiments, the one or more immune checkpoint inhibitors may include an anti-CD28 antibody.

In some embodiments, the therapeutic methods disclosed herein may further comprise administering to a subject an effective amount of one or more LDL/VLDL-lowering medication(s). In some embodiments, the therapeutic methods may comprise pre-treatment with LDL/VLDL-lowering medications.

In some embodiments, the subject is human.

The present disclosure also encompasses methods for delivering a molecular cargo to a cell ex vivo. For example, such methods can comprise administering to a cell an effective amount of a recombinant pseudotyped virus or cell-derived nanovesicle described herein, or a composition comprising the pseudotyped virus or the cell-derived nanovesicle and a carrier and/or excipient. The recombinant fusogenic protein within the recombinant pseudotyped virus or cell-derived nanovesicle can comprise a targeting molecule which targets the cell.

### EXAMPLES

The following examples are provided to further describe some of the embodiments disclosed herein. The examples are intended to illustrate, not to limit, the disclosed embodiments.

### Example 1. Serum Inhibits WT VSV

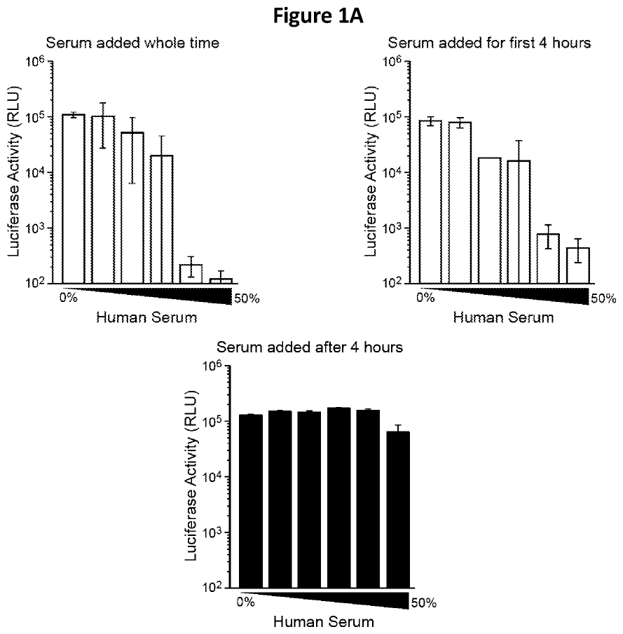

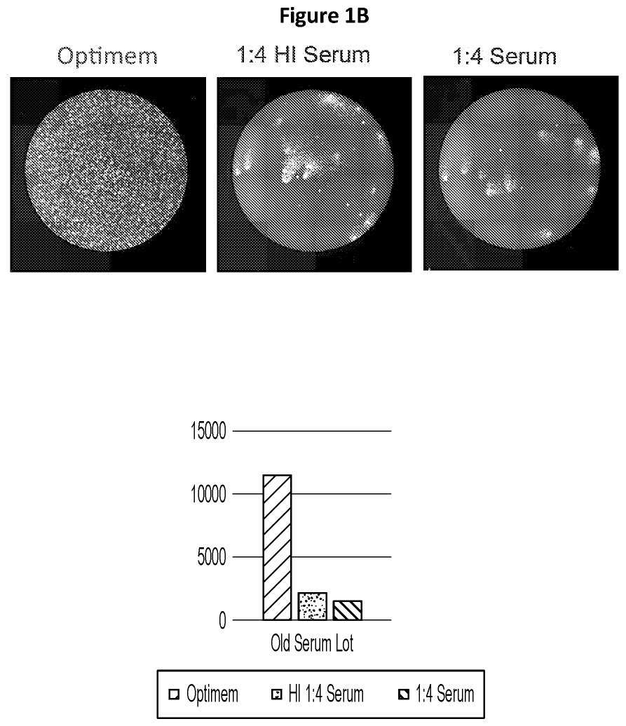

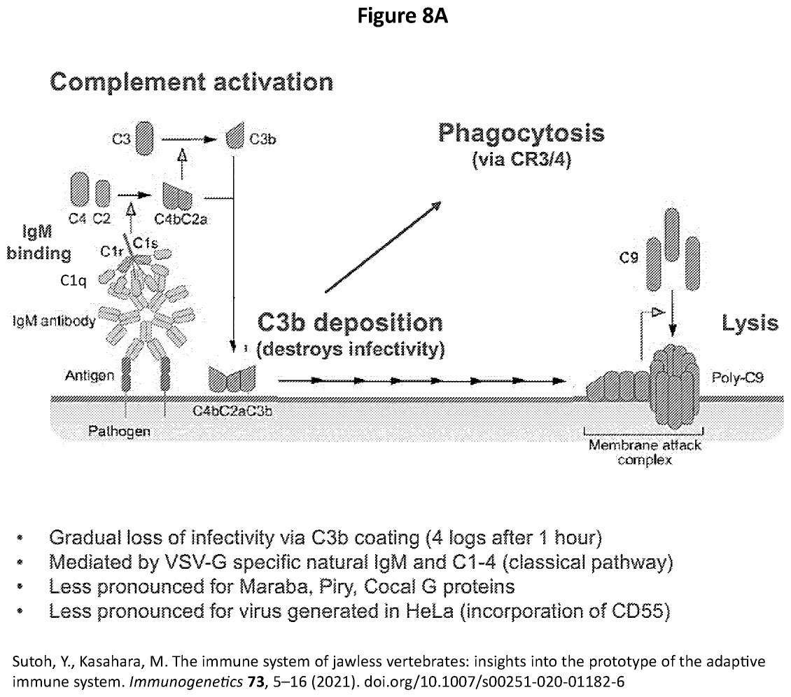

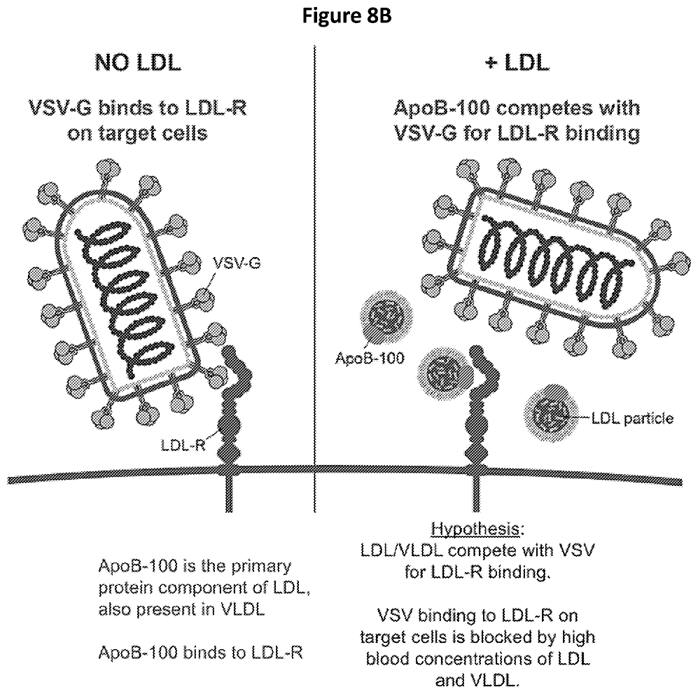

The present Example examined the effects of serum on WT-VSV. A mechanism of VSV activation by serum comprising blockade of LDL receptors by serum lipoproteins is displayed in FIG. 8B. A wild-type (G containing) VSV encoding the luciferase (Fluc) reporter was used to infect Vero cell monolayers in black 96-well plates. Inoculations were carried out for 4 hours at 37° C. using a multiplicity of infection (MOI of 0.01). After 4 hours, the inoculums were removed and replaced with media with or without complement-deficient serum. Infections were left to proceed for an additional 12 hours and luciferase activity, indicative of virus infection, was measured by standard luciferase assay using d-luciferin and a multiplate reader. During the infection/inoculation, increasing concentrations (0%, 3.125%, 6.25%, 12.5%, 25%, or 50%) of pooled human serum were added to the cells at various times, including: 1) during the entire course of infection (FIG. 1A, left panel), 2) during only the first 4 hours of inoculation and then removed (FIG. 1A, middle panel), or 3) only after the 4-hour inoculation (FIG. 1A, right panel). Conditions were tested in triplicate. Results indicate that higher concentrations (25%, 50%) of serum inhibit virus infection, but only when added during the initial 4-hour inoculation. For FIG. 1B, the pooled serum lot used in FIG. 1A was left untreated or incubated at 56° C. for 30 min to inactivate any complement in the serum. The serum lot was later determined to be complement-deficient without the need for heat-inactivation. The serum (or media only) was then added to Vero cell monolayers plated the day before. Wild-type (VSV-G containing) VSV encoding the GFP reporter (VSV-GFP) was added to the cell mixtures. Final serum concentrations were 25% and inoculation MOI was 0.1. After 16 hours, plates were imaged with a Celigo Imaging Cytometry and the number of GFP-positive cells in each well was determined by the Celigo software.

### Example 2. Human Serum Inhibits WT VSV in Human Cell Lines

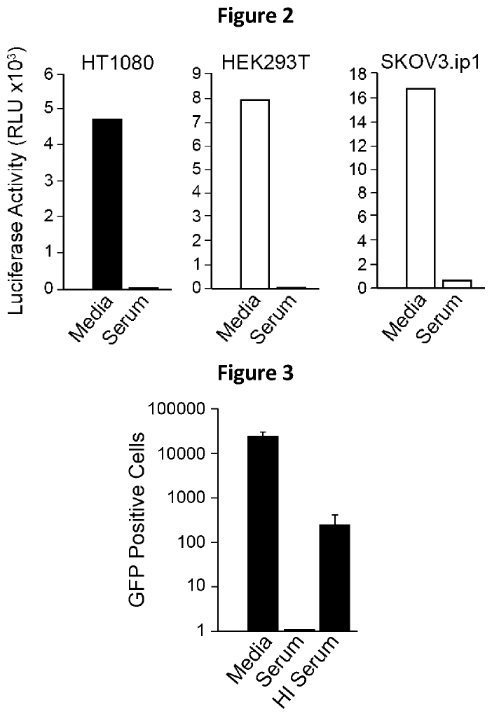

This Example demonstrated inhibition of WT VSV by human serum in various human cell lines. Pooled human serum from the same lot as used in FIG. 1 was mixed with VSV-Fluc virus and then immediately overlaid onto various human cell lines, including HT1080 (FIG. 2, left panel), HEK293T (FIG. 2, middle panel), or SKOV3.ip1 (FIG. 2, right panel), that had been seeded the night before in black 96-well plates. The final serum concentration was 25% and the MOI of infection was 0.1. After 16 hours, luciferase activity was measured by standard luciferase assay.

### Example 3. Heat-Inactivated Serum Inhibits WT VSV in K562 Lymphoblast Cells

The present Example investigated whether heat-inactivated serum inhibits WT VSV in K562 lymphoblast cells. For the experiments, a fresh pool of human serum that contained active complement was used. An aliquot of the serum was thawed at 37° C. to activate complement and then either left untreated or incubated at 56° C. for 30 min to inactivate complement. VSV-GFP was mixed with media, the complement active serum (serum), or heat-inactivated serum (HI serum). After a 30 min incubation at 37° C., the mixes were combined with K562 cells and placed in black 96-well plates. The final MOI for infection was 0.1 and the final serum concentrations were 25%. After 24 hours, plates were imaged with a Celigo Imaging Cytometry and the number of GFP-positive cells in each well was determined (FIG. 3). Data indicated that complement-active serum inhibited VSV-GFP infection of K562 cells. Though complement-inactive serum (e.g., heat-inactivated serum) was less efficient at inhibiting VSV-GFP, the complement-inactive serum did reduce infectivity approximately 100-fold, indicating that there is a heat-stable inhibitory factor in serum that inhibits WT G-containing VSVs.

### Example 4. Heat-Inactivated Serum Inhibits WT VSV in Multiple Human Cell Lines

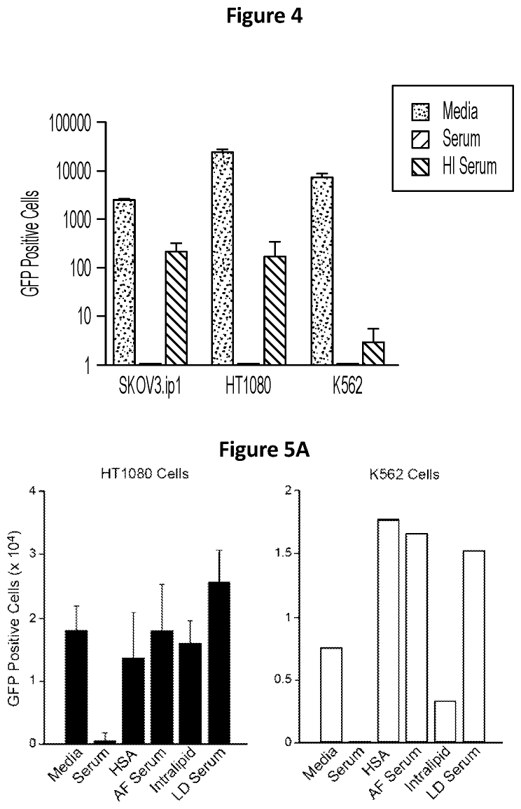

The present Example investigated whether heat-inactivated serum inhibits WT VSV in various human cell lines. Media alone, complement-active human serum pool (serum) or complement-inactivated human serum pool (HI serum) was added to different human cell lines in black 96-well plates. Cells used include SKOV3.ip1, HT1080, and K562. Next, VSV-GFP was added to the cells at an MOI=0.1, in a volume that resulted in a final serum concentration in the wells of 25%. After 16 hours, plates were imaged with a Celigo Imaging Cytometer and the number of GFP-positive cells in each well was determined by the Celigo software (FIG. 4). Data from the K562 cells indicated that when HI serum is mixed with the cells prior to addition of virus, it more potently inhibits infection compared to when serum and virus are mixed prior to addition of cells (see, e.g., FIG. 4 compared to FIG. 3). Additionally, in multiple human cell lines, complement-active serum inhibits VSV even without pre-incubation of serum with virus. This Example showed that complement-inactivated serum inhibited VSV infection in multiple tested human cell lines, confirming the presence of a heat-stable inhibitory serum component.

### Example 5. Serum Depleted of Lipoproteins does not Possess Inhibitory Activity

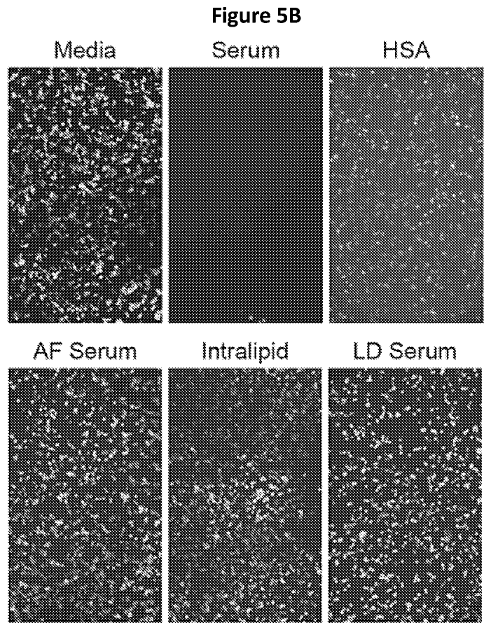

This Example explored the inhibitory activity of lipoprotein-depleted serum on WT-VSV GFP. For the experiment, media alone, heat-inactivated serum (serum), human serum albumin (HSA), artificial serum (AF serum), Intralipid, or lipoprotein-depleted serum (LD serum) was added to HT1080 or K562 human cell lines in black 96-well plates. VSV-GFP was added to the cells at an MOI=0.1, in a volume that resulted in a final serum or human serum albumin (HSA) concentrations in the wells of 25% or a final Intralipid concentration of 250 μg/mL. After 16 hours, plates were imaged with a Celigo Imaging Cytometer and the number of GFP-positive cells in each well was determined by the Celigo software (FIG. 5A). Representative fluorescence microscopy images were taken at 16 hours for the HT1080 cells (FIG. 5B). Data showed that lipoproteins, but not HSA or lipids, are primarily responsible for the heat-stable inhibitory activity of serum.

### Example 6. LDL and VLDL, but not HDL Inhibit WT VSV

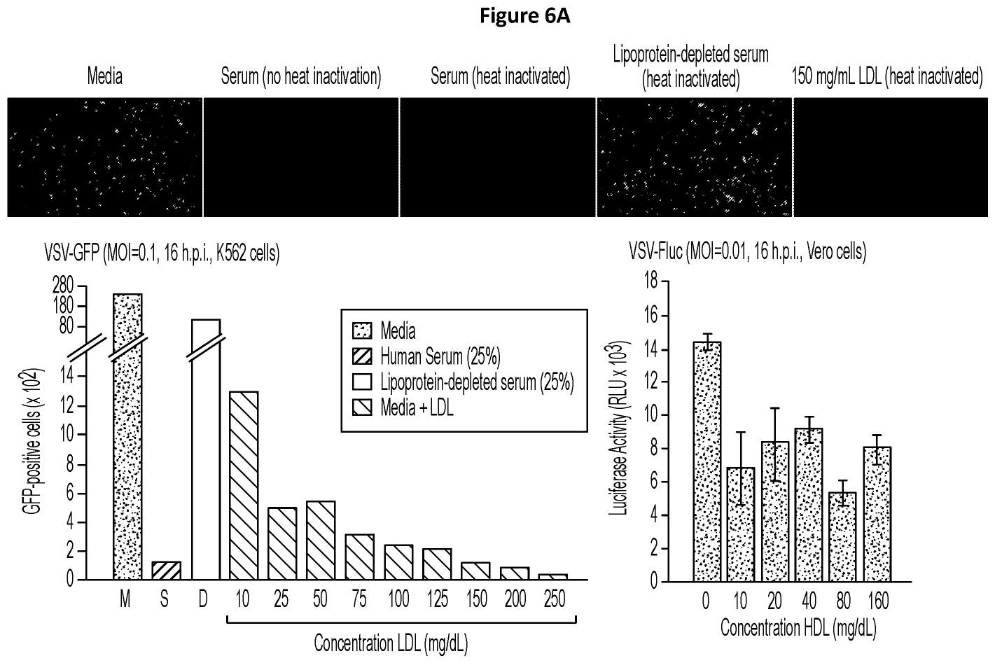

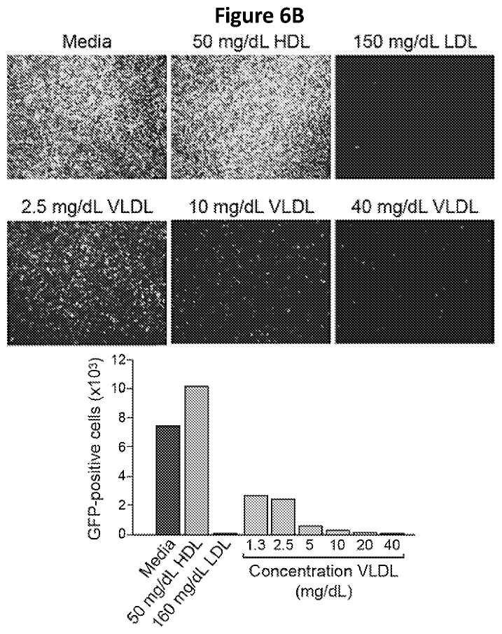

This Example investigated whether LDL, VLDL and/or HDL inhibit WT VSV. For some experiments, K562 cells were mixed with VSV-GFP and immediately overlaid into wells of various test conditions, including media alone (Media; M), human serum pool (S) (from FIG. 1), lipoprotein-depleted human serum pool (D), or media containing various concentrations of purified human LDL, HDL, or VLDL. The MOI of infection was 0.1. Final serum concentrations were 25% and final LDL, HDL, and VLDL concentrations in mg/dL were as indicated in FIG. 6. After 16 hours plates were imaged by fluorescence microscopy (FIG. 6A, top panel and FIG. 6B, left panel) and after 24 hours plates were imaged with a Celigo Imaging Cytometry and the number of GFP-positive cells in each well was determined by the Celigo software (FIG. 6A, bottom left panel and FIG. 6B, right panel). In a separate experiment, VSV-Fluc was used to inoculated Vero cells (MOI=0.01) in wells that contained increasing amounts of HDL spiked into lipoprotein-depleted serum. Luciferase activity (indicative of VSV-Fluc infection) was measured at 16 hours (FIG. 6A, bottom right panel). Data indicated that LDL and VLDL inhibit VSV-GFP in a dose-dependent manner. However, HDL does not inhibit VSV infection.

### Example 7. Heat Resistance of LDL and VLDL Inhibitory Activity

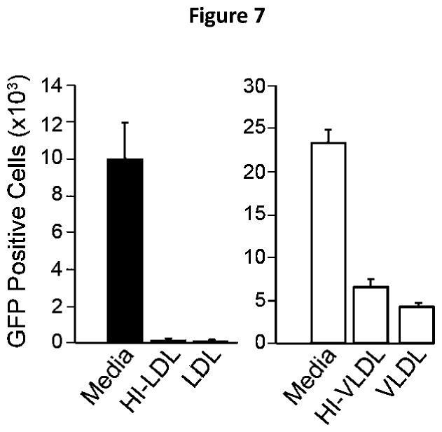

The present Example interrogated heat resistance of LDL- and VLDL-associated inhibition of VSV-GFP. Purified commercial human LDL (FIG. 7, left panel) or VLDL (FIG. 7, right panel) was heat inactivated for 30 min at 56° C. (HI-LDL or HI-VLDL) or left untreated (LDL or VLDL). K562 cells were mixed with VSV-GFP and immediately overlaid into wells of the various test conditions, including media alone (Media) or heat-inactivated or untreated LDL or VLDL. Final virus MOI was 0.1 and final concentrations of LDL and VLDL were 150 mg/dL and 5 mg/dL, respectively. After 24 hours, plates were imaged with a Celigo Imaging Cytometer and the number of GFP-positive cells in each well was determined by the Celigo software (FIG. 7). Data indicated that LDL and VLDL inhibition of VSV-GFP is resistant to heat inactivation.

### Example 8. Design of Retargeted VSV Glycoprotein

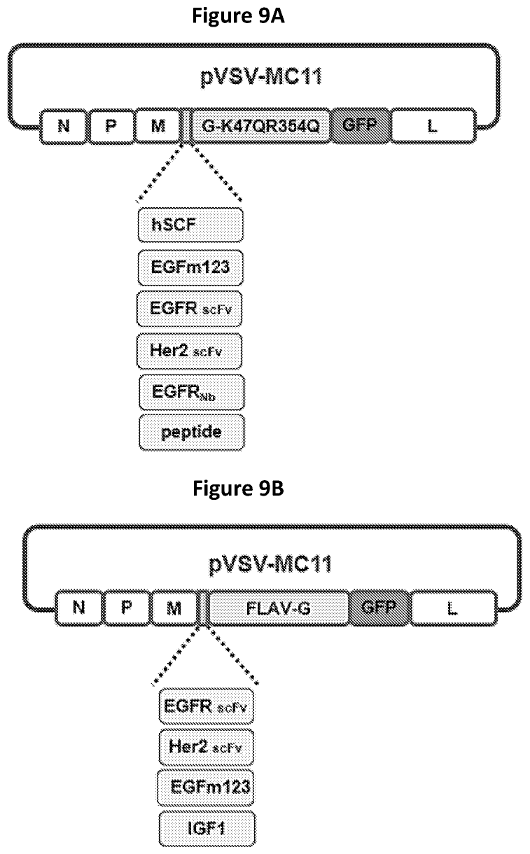

The present Example discusses design of retargeted VSV glycoproteins. The VSV-G protein contained a signal peptide (SP) that could be proteolytically cleaved following translation. The targeting molecules of the present Example were single chain fragment variable fragments (scFvs) antibodies raised to the human epidermal growth factor receptor 2 (HER2) or epidermal growth factor receptor (EGFR) or natural ligands such as, but not limited to, EGFm123 (modified EGF) or nanobodies or peptides, which were appended to the amino-terminus of the G protein, with or without a flexible linker. Two mutations in the G polypeptide were incorporated at amino acid positions 47 and 354 (K47Q/R354Q) (see, e.g., FIG. 9A). The same targeting molecules were appended to the N-terminus of alternate Rhabdovirus Flanders G (FLAV-G) described herein, with or without a flexible linker (FIG. 9B).

### Example 9. VSV-G Harboring Blinding Mutations and an Anti-HER2 scFv Specifically Fuses HER2-Expressing Cell Lines

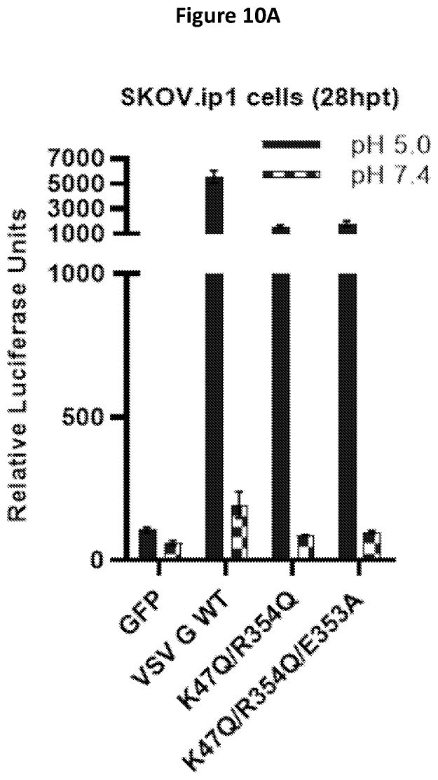

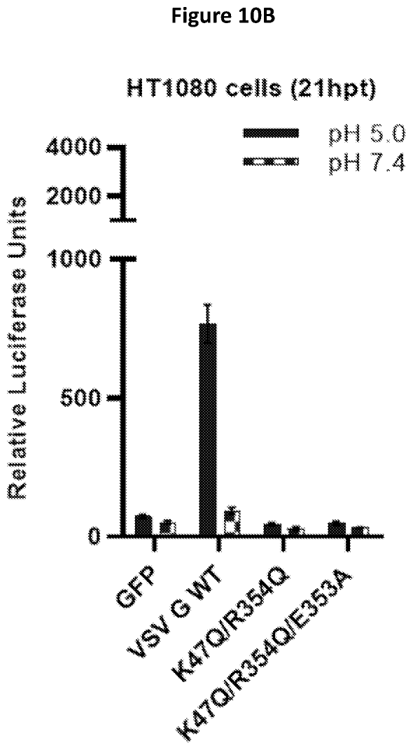

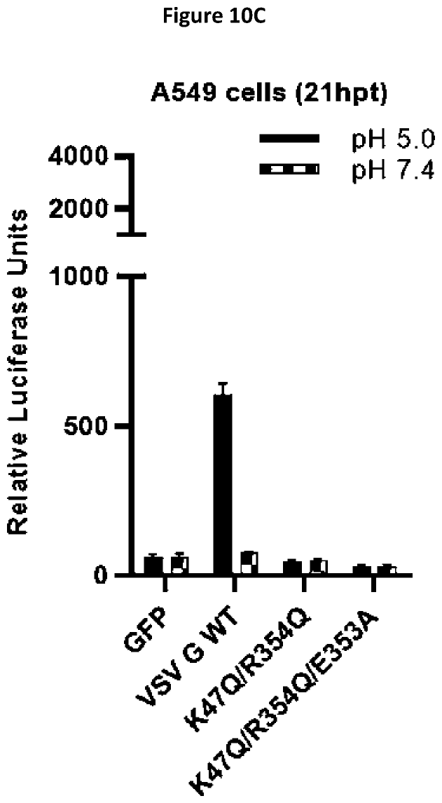

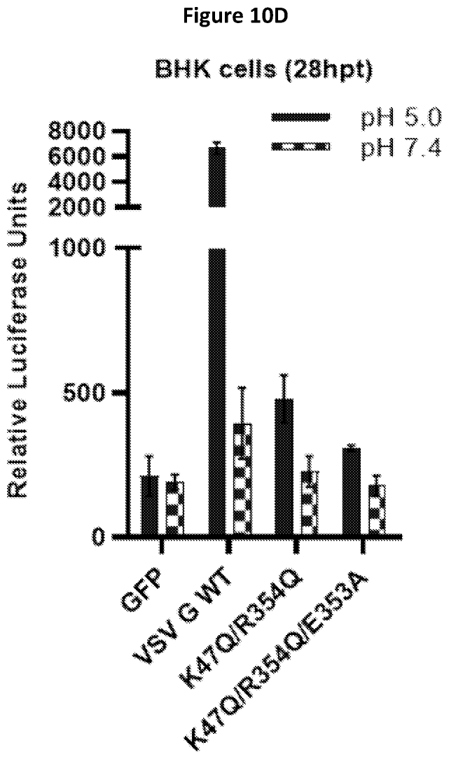

This Example assessed fusion of VSV-G comprising blinding mutations and an anti-HER2 scFv with HER2-expressing cell lines. For the experiments, SKOV3.ip1 (FIG. 10A), HT1080 (FIG. 10B), A549 (FIG. 10C), and BHK-21 (FIG. 10D) cells stably expressing DSP-1 or DSP-2 reporters were co-cultured. The next day the cells were transfected with plasmids expressing GFP, wild type VSV-G, or VSV-G harboring an N-terminal anti-HER2 scFv and either two or three mutations aimed at ablating the glycoprotein's interaction with LDLR (see, e.g., FIG. 8A). One day after transfection, the cells were treated with pH 5.0, to mediate fusion, or neutral phosphate buffered saline (PBS) for 2 minutes, then fresh media was added containing the luciferase substrate, EnduRen. Renilla luciferase activity was measured 4 hours after the addition of the substrate. HER2 expression levels in the transfected cells were measured by flow cytometry and the signal to noise (S/N) ratio was determined, as described in Table 3. Data indicated that modified VSV-G fused with HER2-expressing cells expressing high levels of HER2.

TABLE 3
HER2 Receptor Levels Determined by Flow Cytometry

| Cell Line | S/N Ratio |
|---|---|
| SKOV3ip.1 | 338 |
| HT1080 | 2.77 |
| A549 | 0.52 |
| BHK | 0.4 |

### Example 10. Infection of Blinded and Retargeted VSV-G Correlates with HER2 Receptor Levels

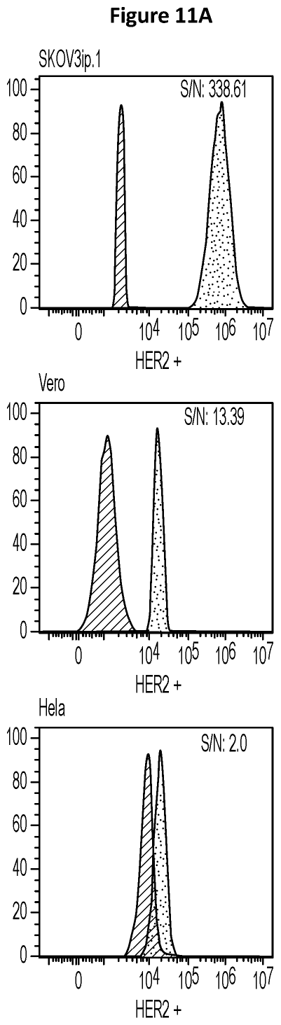

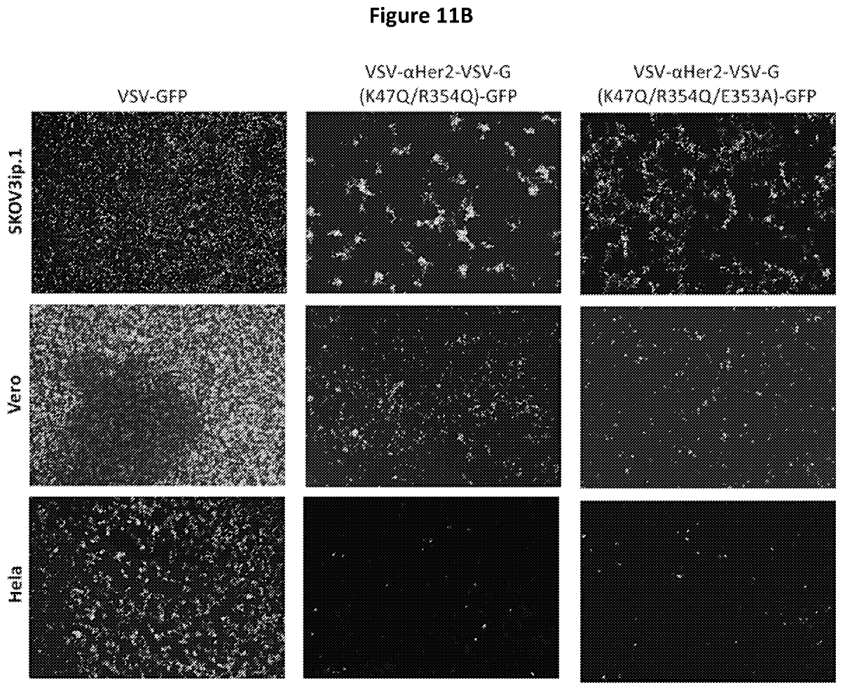

This Example was designed to assess the relationship between the level of infection of blinded and retargeted VSV-G and HER2 expression. Expression of the HER2 receptor was determined in SKOV3.ip1, Vero, and Hela cells by flow cytometry (FIG. 11A). SKOV3.ip1, Vero and Hela cells were infected with VSV-GFP (WT VSV), VSV-α HER2-VSV-G(K47Q/R354Q)-GFP, or VSV-αHER2-VSV-G(K47Q/R354Q/E353A)-GFP. Images were captured by fluorescence microscopy (FIG. 11B). Data indicated that the level of infection for HER2-retargeted viruses correlates with the cellular level of HER2 expression.

### Example 11. Soluble Anti-HER2 scFv Selectively Inhibits HER2 Retargeted, LDLR Mutant Virus Infection and Spread in SKOV3Ip.1 Cells

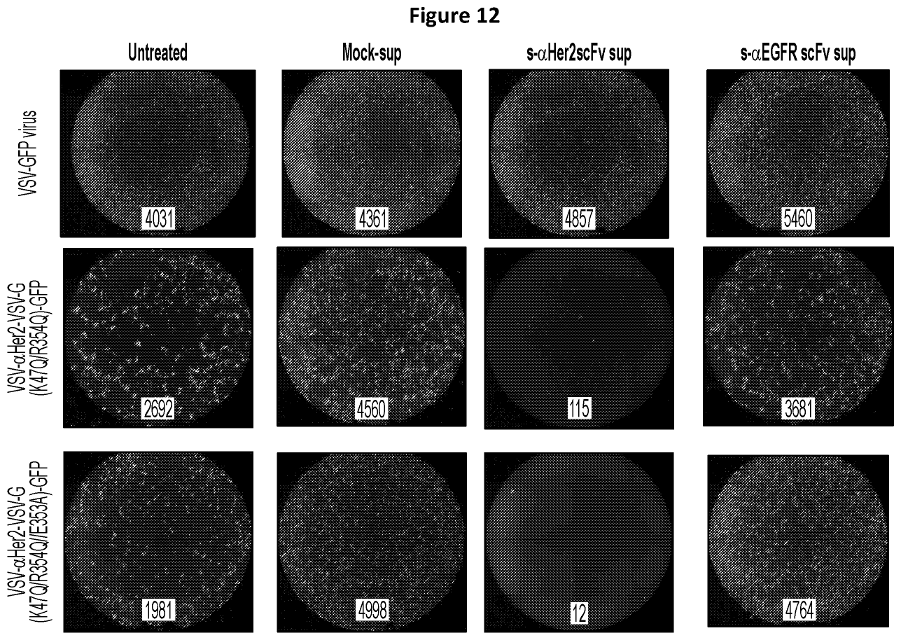

This Example tested inhibition of HER2 retargeted LDLR mutant virus infection and spread in SKOV3ip.1 cells with soluble anti-HER2 scFv. SKOV3ip.1 cells, which express high levels of HER2, were infected with VSV-GFP, VSV-αHER2-VSV-G (K47Q/R354Q)-GFP, or VSV-αHER2-VSV-G (K47Q/R354Q/E353A)-GFP. Simultaneously, the cells were treated with media from cultures of cells that had been transfected with plasmids expressed GFP (Mock-sup) or a secreted form of an anti-HER2 or anti-EGFR scFv. Virus infection and spread was monitored by imaging and counting the number of GFP-positive cells using a Celigo Imaging Cytometer (FIG. 12). Data indicated that secreted anti-HER2 selectively inhibited HER2-retargeted viruses.

### Example 12. VSV-G can Target the EGF or HER2 Receptors in the Same Cell Type

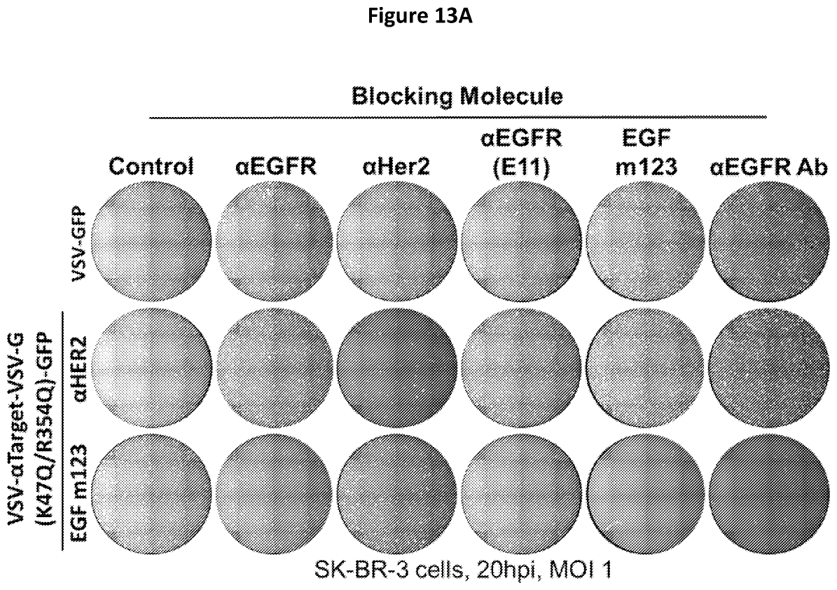

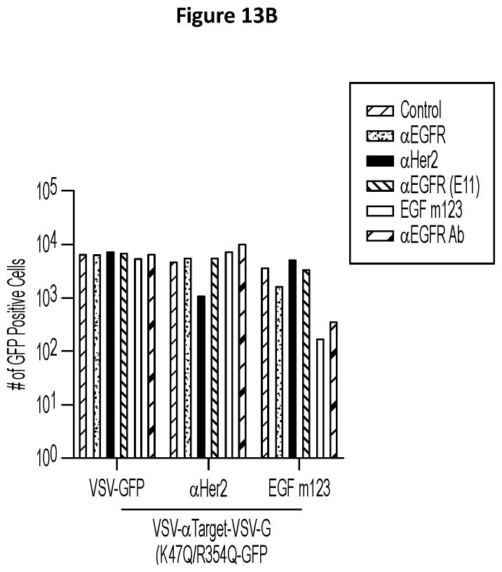

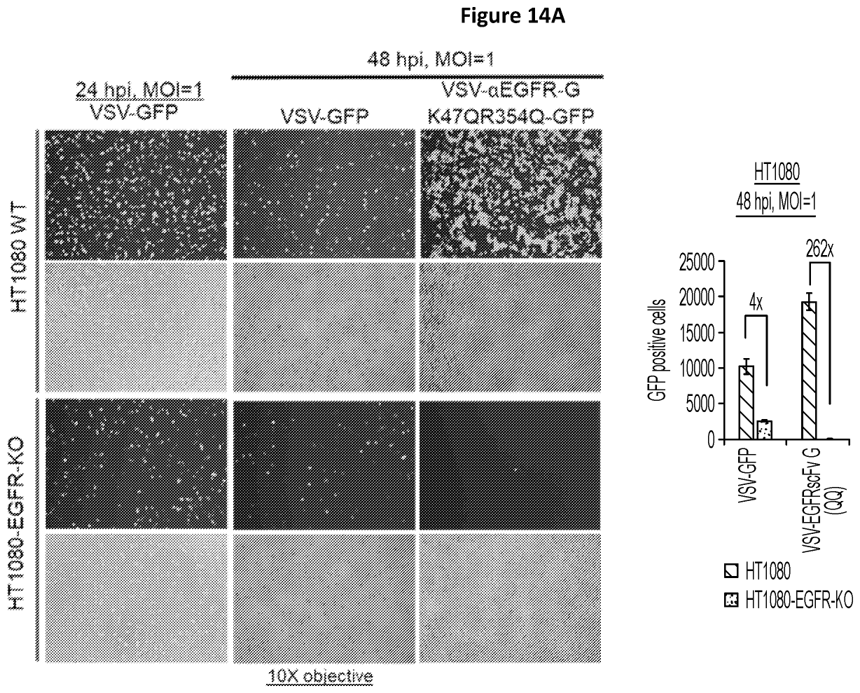

This Example was designed to determine whether retargeted VSV-Gs were capable of targeting EGF or HER2 receptors in the same cell type. SK-BR-3 cells that express both EGFR and HER2 were infected with control VSV-GFP or VSV containing two blinding mutations and a targeting molecule to EGFR or HER2 (EGFm123 or an scFv raised to HER2) at a MOI of 1. Simultaneously, the cells were treated with media (control) or blocking molecules. Blocking molecules were generated by transfecting HEK293T cells with plasmids expressing the scFv or modified EGFm123. The anti-EGFR antibody used was a reconstituted antibody comprised of two copies of the same anti-EGFR scFv. Twenty hours after infection, the cells were imaged by a Celigo Imaging Cytometer (FIG. 13A). The number of GFP-positive cells was determined by Celigo software (FIG. 13B). Data indicated infection of HER2- and EGFR-expressing cells by HER2- and EGFR-targeted VSVs is reduced by HER2 and EGFR blocking molecules, respectively.

### Example 13. Specificity of Retargeted VSV-αEGFR-G K47QR354Q-GFP in HT1080-EGFR-KO and HEK-293T-EGFR-KO Cells

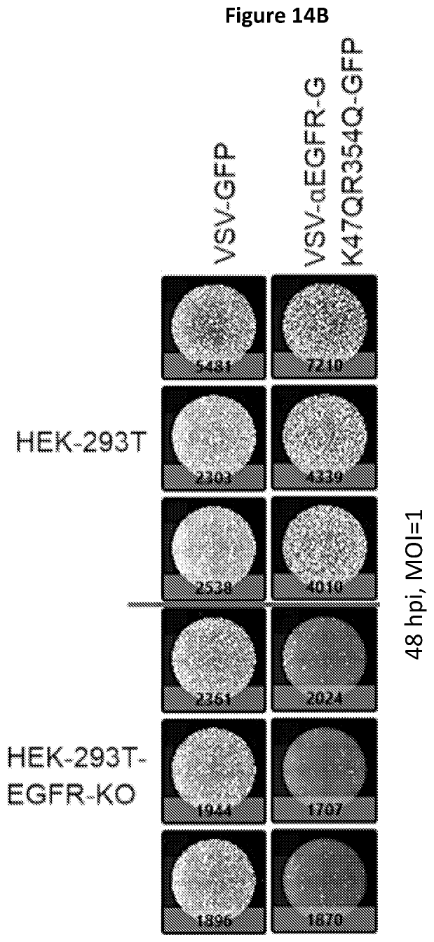

The present Example tested the specificity of retargeted VSV-αEGFR-G K47QR354Q-GFP in HT1080-EGFR-knock out (KO) and HEK-293T-EGFR-KO cells. For infection in HT10180 cells, HT1080 wildtype (WT) or HT1080-EGFR-KO cells were infected with VSV-GFP (control) or VSV-αEGFR-G K47QR354Q-GFP at an MOI=1. The GFP images were taken at 24 and 48 hpi with an 10× objective lens using a NIKON fluorescence microscope (FIG. 14A, left), and the number of GFP-positive cells were quantified by imaging of the plates using a Celigo Imaging Cytometer (FIG. 14A, right). For infection in HEK-293T cells, HEK-293T wildtype (WT) or HEK-293T-EGFR-KO cells were infected with VSV-GFP (control), or VSV-αEGFR-G K47QR354Q-GFP at an MOI=1. GFP images were taken at 48 hpi by the Celigo Imaging Cytometer (FIG. 14B). Data indicated that EGFR retargeted viruses robustly infect HT1080 and HEK293T cells that express EGFR, but infection is reduced in HT1080 and HEK293T cells that lack EGFR.

### Example 14. Testing the Specificity of Retargeted VSV on a K562 Cell Panel

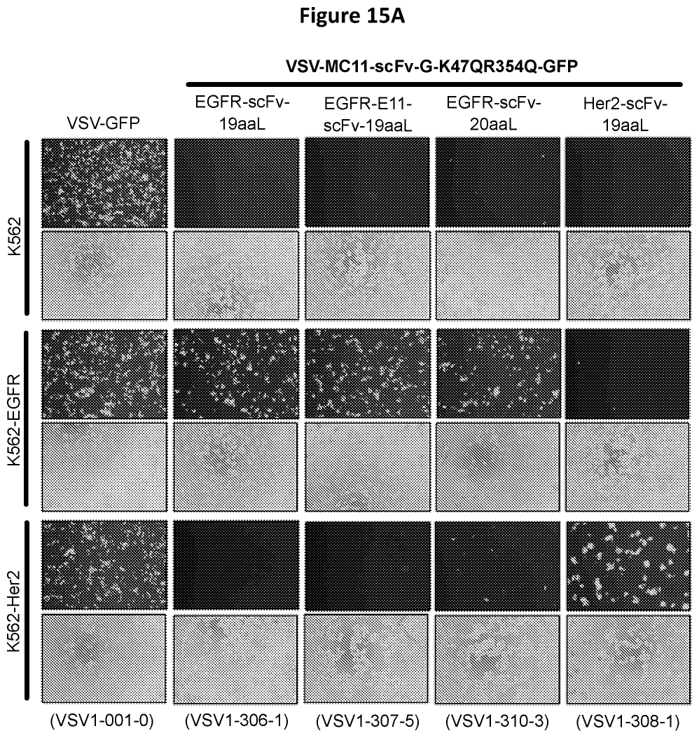

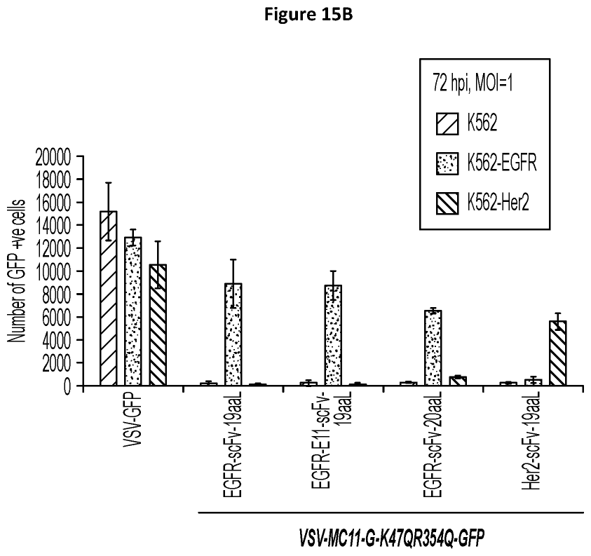

This Example investigated the specificity of retargeted VSVs on a K562 cell panel. K562 parental or EGFR/HER2 receptor expressing cells were infected with VSV-GFP (as a control) or retargeted VSVs with modified linker sequences (19 aa linker: RAAA(G₄S)₃ (SEQ ID NO: 170); 20 aa linker: KRAAASGGS(G₄S)₂GPK (SEQ ID NO: 174)) at an MOI=1. Fluorescence microscopy images were taken at 72 hpi with an 10× objective lens using a NIKON microscope (FIG. 15A). The number of GFP-positive cells was quantified by a Celigo Imaging Cytometer (FIG. 15B). Flow cytometry was performed on the K652 cell panel, as described in Table 4. The receptor expression levels were quantified and the signal to noise (S/N) ratio was determined. Results indicated specific targeting of EGFR- and HER2-retargeted viruses to EGFR- and HER2-expressing K562 cells, respectively, with little background on cells not expressing the targeted receptor.

TABLE 4
Flow Cytometry for EGFR/HER2 Levels

| Cells | S/N Ratio | HER2 (S/N) |
|---|---|---|
| K562 | 1.0 | 0.89 |
| K562-EGFR | 2.833 | — |
| K562-HER2 | — | 2.941 |

### Example 15. Incorporation of Targeting Molecules EGFm123 or hSCF in Virus Particles

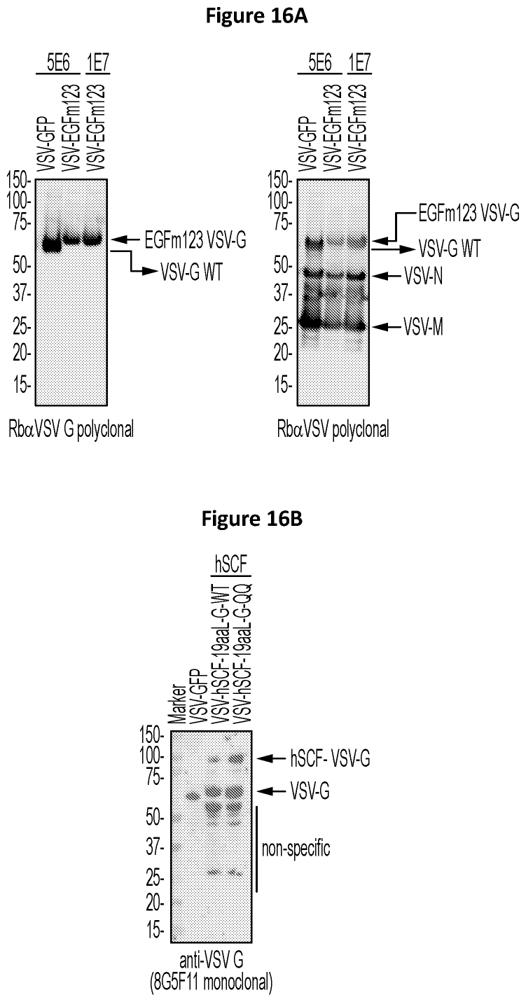

The present Example was designed to assess incorporation of targeting molecules EGFm123 or hSCF in virus particles. For western blotting of EGFm123-displaying virus particles, 5×10⁶ TCID₅₀/mL virus particles of VSV-GFP or VSV-EGFm123-G K47QR354Q-GFP, or 1×10⁷ TCID₅₀/mL of VSV-EGFm123-G K47QR354Q-GFP virus were pelleted and subjected to western blotting with anti-VSV-G or anti-VSV polyclonal antibodies (FIG. 16A). For western blotting of hSCF displaying virus particles, 9.8×10⁶ TCID₅₀/mL of VSV-MC11-hSCF-20aaL(F)-VSV G-WT-GFP or 1.17×10⁸ TCID₅₀/mL of VSV-MC11-hSCF-20aaL(F)-VSV G-QQ-GFP were pelleted and subjected to western blotting with anti-VSV-G (8G5F11) monoclonal antibody (FIG. 16B). Data indicated the EGFm123 and SCF are incorporated into virus particles.

### Example 16. Specificity of Retargeted VSV Displaying Smaller Targeting Molecules

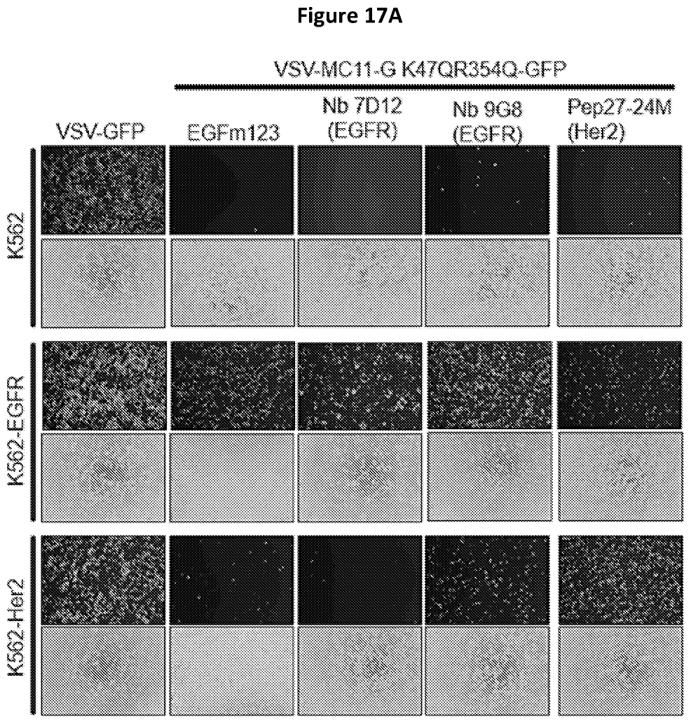

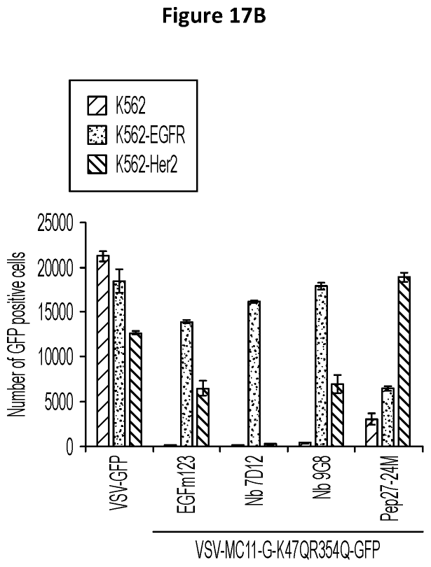

The present Example interrogated the specificity of retargeted VSV displaying K562 displaying smaller targeting molecules such as nanobodies (e.g., Nb 9G8) and peptides (e.g., Pep27-24M). Parental, K562-EGFR, or K562-HER2 cells were infected with VSV-GFP (as a control) or the indicated retargeted VSVs at an MOI of 1.0. Fluorescence microscopy images were acquired at 42 hpi with an 10× objective lens using a Nikon microscope (FIG. 17A). The number of GFP-positive cells were quantified by the Celigo Imaging Cytometer (FIG. 17B). Data indicated specific EGFR targeting of Nb 7D12, and partial targeting of Nb 9G8 and Pep27-24M.

### Example 17. VSV-EGFm123 Infection in EGFR Knockout Cell Lines

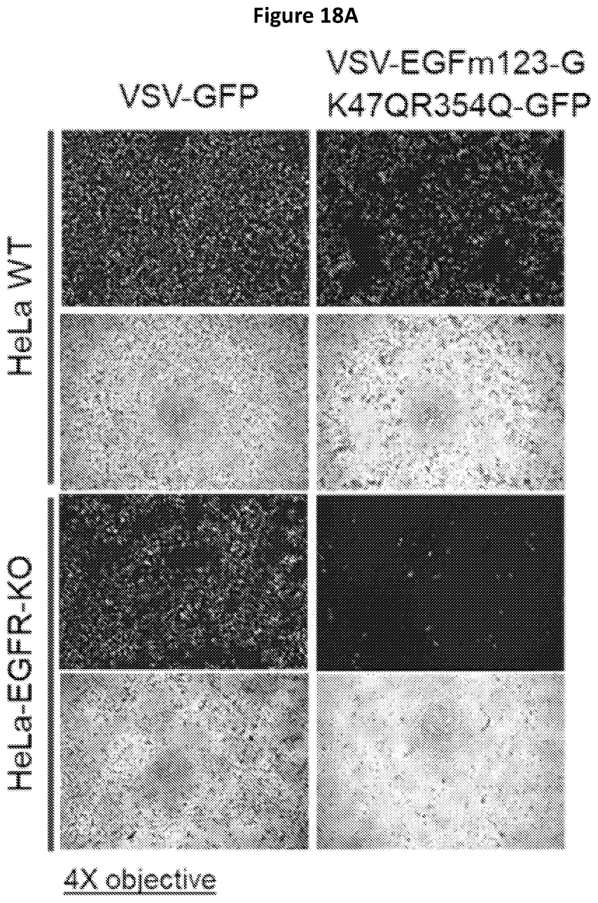

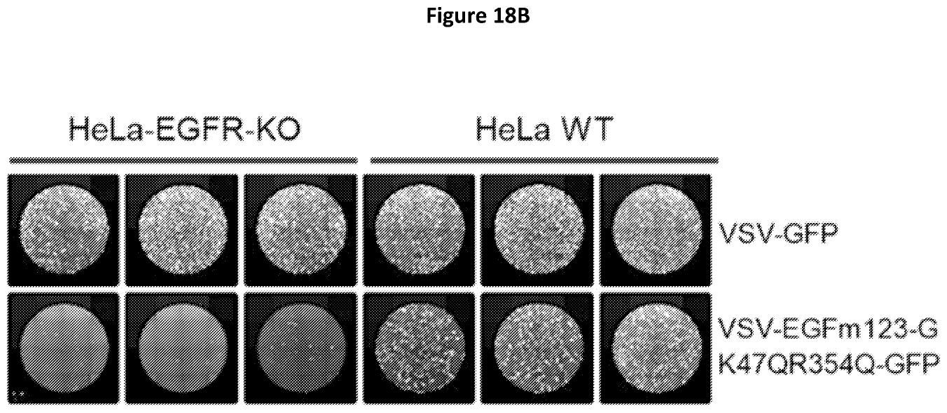

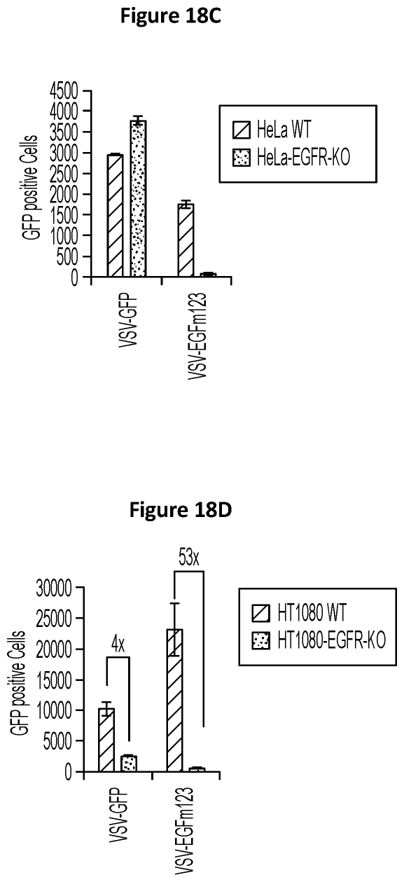

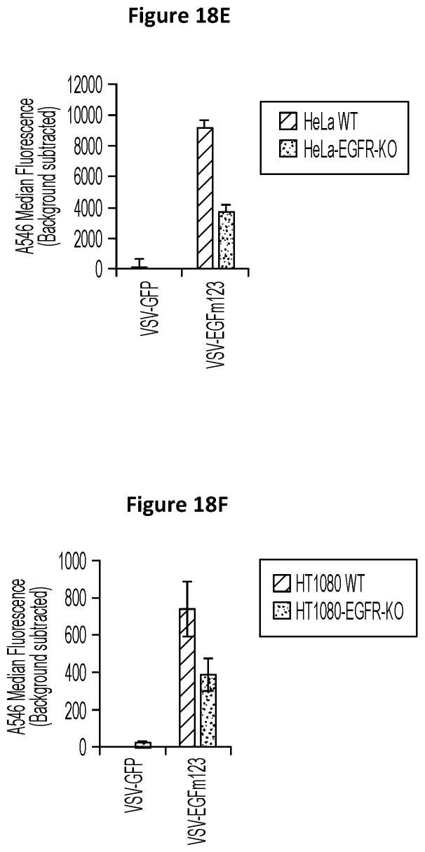

This Example tested VSV-EGFm123 infection in EGFR knockout (KO) cell lines. For the experiments, cells were infected with VSV-GFP (control) or VSV-EGFm123 virus with MOI=1.0 for 48 hours. GFP and phase contrast images of HeLa WT or HeLa-EGFR-KO cells were acquired using a 4× objective with a Nikon microscope (FIG. 18A). GFP images were taken with a Celigo Imaging Cytometer (FIG. 18B) and the infection level was quantified as the number of GFP-positive cells for the HeLa cell panel (FIG. 18C) or HT1080 cell panel (FIG. 18D). The ability of VSV-EGFm123 to bind its receptor was determined using HeLa or HT1080 WT or EGFR-KO cells. 1×10⁶ cells were incubated with VSV-GFP or VSV-EGFm123 virus at MOI=10 for 2 hr at 4° C. while rocking. The cells were fixed with 2% paraformaldehyde, followed by staining with a PE (phycoerythrin)-conjugated VSV-G antibody. The cells were analyzed by flow cytometry (FIGS. 18E-18F). Data indicated that EGFm123 viruses are targeted to enter cells through the EGFR.

### Example 18. VSV-G-O-EGF-m123 Virus is Resistant to Inhibition by LDL

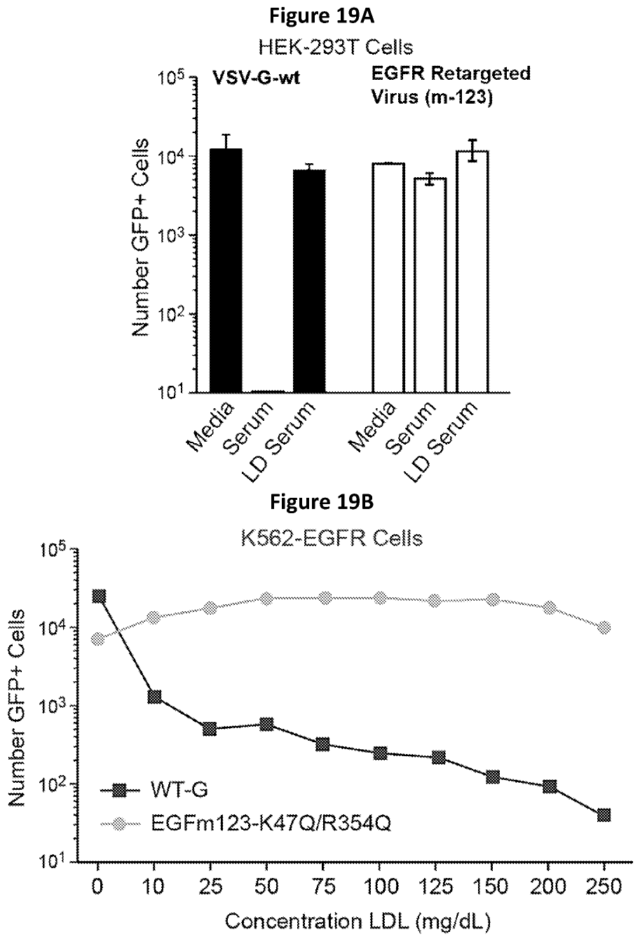

The Example interrogated VSV-G-QQ-EGF-m123 virus inhibition by LDL. VSV-GFP (VSV-G-WT) or VSV-GFP with G containing the K47Q/R354Q mutation and appended to EGFm123 (EGFR retargeted virus) was incubated with media alone (control), human pooled serum (complement deficient lot), or pooled lipoprotein-depleted human serum. Immediately the mixtures were overlaid onto HEK-293T cells plated the day before in black 96-well plates. Infections were carried out at an MOI of 0.1 for VSV-G-WT and MOI of 1.0 for EGFR retargeted viruses, and final serum concentrations were 25%. After 24 hours, plates were imaged with a Celigo Imaging Cytometer and the number of GFP-positive cells per well was quantitated (FIG. 19A). VSV-GFP (VSV-G-WT) or VSV-GFP with G containing the K47Q/R354Q mutation and appended to EGFm123 (EGFm123-K47Q/R354Q) was mixed with K562-EGFR cells and immediately added to wells containing media alone (control) or increasing concentrations of purified human LDL. Infections were carried out at an MOI of 0.1 for WT-G and 1.0 for EGFm123-K47Q/R354Q viruses, with final LDL concentrations as indicated in FIG. 19. After 24 hours, plates were imaged with a Celigo Imaging Cytometer and the number of GFP-positive cells per well was quantitated (FIG. 19B). Data indicated that VSV retargeted to EGFR through EGFm123 is resistant to inhibition by LDL, the heat-stable inhibitory component in human serum.

### Example 19. VSV-G-QQ-EGF-m123 Virus is Resistant to Inhibition by VLDL

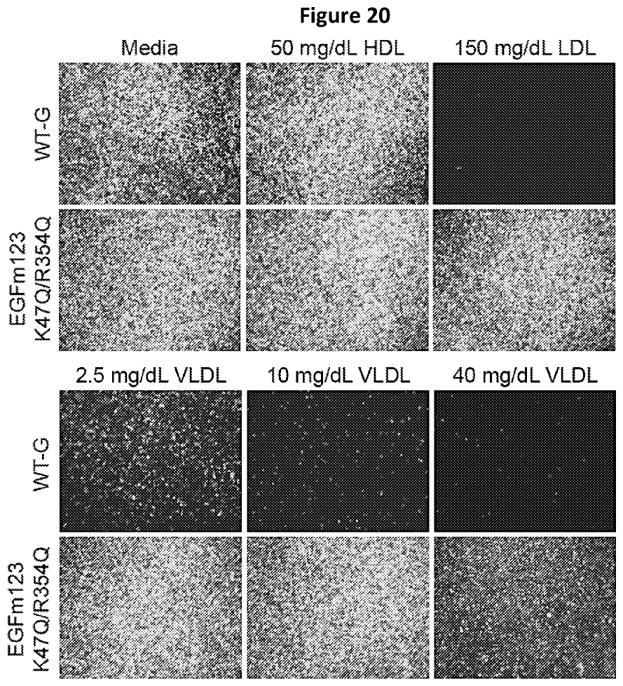

This Example tested VSV-G-QQ-EGF-m123 virus inhibition by VLDL. VSV-GFP (VSV-G-wt) or VSV-GFP with G containing the K47Q/R354Q mutation and appended to EGFm123 (EGFm123-K47Q/R354Q) was mixed with K562-EGFR cells and immediately added to wells containing media alone (control) or purified human HDL, LDL, or VLDL. Infections were carried out at an MOI of 0.1 for WT-G and 1.0 for EGFm123-K47Q/R354Q viruses, with final lipoprotein concentrations as follows: 50 mg/dL HDL, 150 mg/dL LDL, 2.5 mg/dL VLDL, 10 mg/dL VLDL, 40 mg/dL VLDL. After 24 hours, plates were imaged by fluorescence microscopy (FIG. 20). Data indicated that VSV retargeted to EGFR through EGFm123 is resistant to inhibition from VLDL.

### Example 20. Binding of WT VSV but not EGFR Retargeted VSV is Reduced by LDL

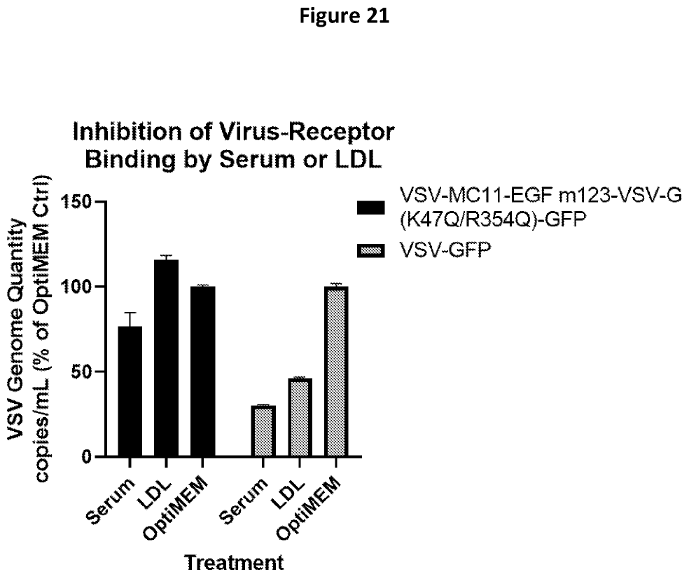

The present Example interrogated the effects of LDL on the binding of EGFR retargeted VSV versus WT VSV. For the experiment, VSV-GFP (VSV-G-wt) or VSV-GFP with G containing the K47Q/R354Q mutation and appended to EGFm123 (VSV-MC11-EGFm123-VSV-G(K47Q/R354Q)-GFP) was mixed with media alone (OptiMEM), pooled human serum (serum) or LDL and immediately overlaid onto HT1080 cells at 4° C. to allow for VSV binding but not entry. Infections were carried out at an MOI of 10. Serum concentration was 25% and LDL concentration was 150 mg/dL. After 1.5 hours, cells were washed three times with cold buffered saline. RNA was then extracted from the cells, including any RNA from bound virus or virus that entered the cells, and samples were subjected to qRT-PCR using primers and probes specific for the VSV genome (IDT) (FIG. 21).

To detect VSV genome the following primers and probes were used:

gVSV-NIP-F:
(SEQ ID NO: 149)
GTCAGAATTTGACAAATGACCC
gVSV-NIP-R:
(SEQ ID NO: 150)
GTGAGATTATCCATGATATCTGTTAG
gVSV-NIP-Probe:
(SEQ ID NO: 151)
CTCGAGTCACCTATTATATATTATGCTACATATG-HEX

A cellular control gene was also detected (RPP30) using the following:

RPP30-F:
(SEQ ID NO: 152)
AGATTTGGACCTGCGAGCG
RPP30-R:
(SEQ ID NO: 153)
GAGCGGCTGTCTCCACAAGT
RPP30 Probe:
(SEQ ID NO: 154)
TTCTGACCTGAAGGCTCTGCGCG-FAM

Data represent the number of VSV genome copies relative to the number of copies from the media only control for the virus. Data indicated that human serum and LDL had no effect on binding of VSV retargeted to EGFR through EGFm123 to HT1080 cells but did reduce binding of WT VSV to HT1080 cells.

### Example 21. Sensitivity of Retargeted VSV-Gs to Human Serum and LDL

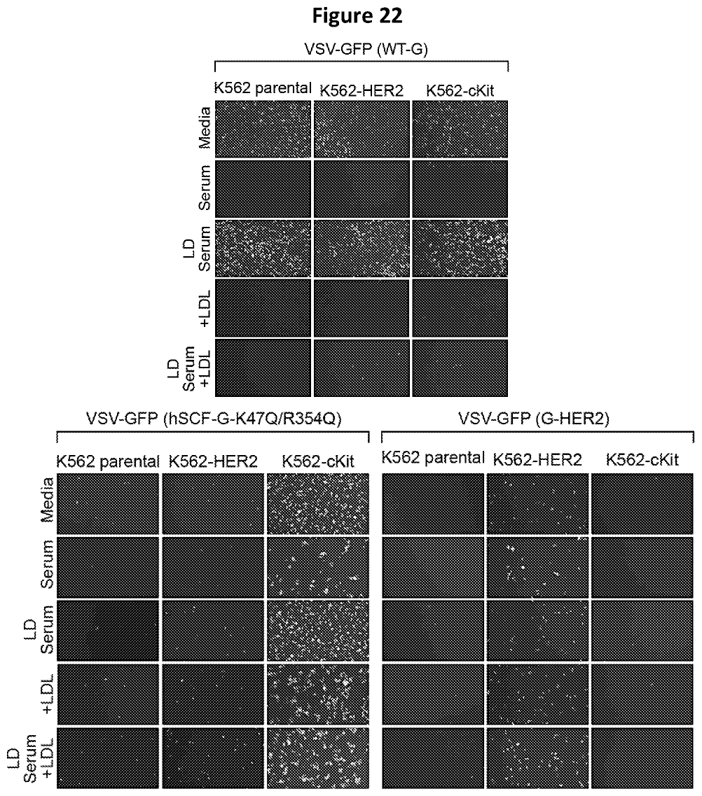

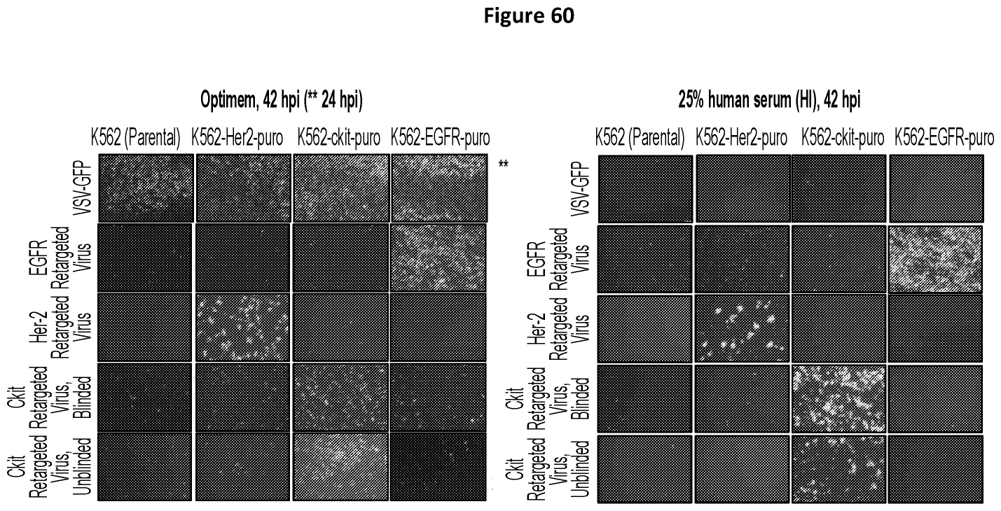

The present Example tested the sensitivity of retargeted VSV-Gs to human serum and LDL. For the experiment, VSV-GFP (WT-G), or VSV-GFP with G containing the K47Q/R354Q mutations and appended to either human stem cell factor (hSCF) scFv (VSV-GFP (hSCF-G-K47Q/R354Q)), HER2 scFv (VSV-GFP (G-HER2)), or EGFm123 (see, e.g., FIG. 60) were mixed with various cells from a K562 cells panel, including K562 parental cells which do not contain HER2, EGFR, or cKit receptors, K562-HER2 cells, K562-EGFR, or K562-cKit cells. Immediately, mixes were added to wells containing media alone (control), human pooled serum (serum), human pooled lipoprotein-depleted serum (LD Serum), media containing LDL (+LDL), or human pooled lipoprotein-depleted serum with LDL (LD Serum+LDL). Infections were carried out at an MOI of 0.1 for WT-G and 1.0 for the retargeted viruses, with final serum concentrations of 25% and LDL concentrations of 150 mg/dL. After 24 hours, fluorescent photos were taken at 100× magnification using an Olympus microscope (FIG. 22 and FIG. 60). Data indicated that SCF, EGFR, or HER2-retargeted viruses are resistant to serum/LDL inhibition.

### Example 22. Sensitivity of Retargeted VSV-Gs to Human Serum and LDL in PC3 Cells

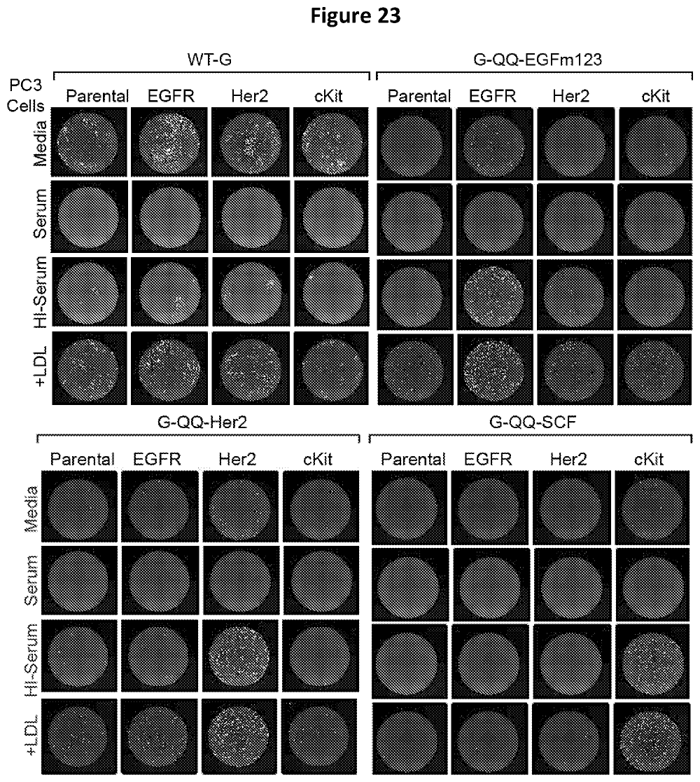

The present Example tested the sensitivity of retargeted VSV-Gs to human serum and LDL using prostate cancer PC3 cells. For the experiments, VSV-GFP (WT-G), or VSV-GFP with G containing the K47Q/R354Q mutations and appended to either EGFm123 (G-QQ-EGFm123), hSCF (G-QQ-SCF), or HER2 scFv (G-QQ-HER2) were mixed with media alone (control), fresh (i.e., complement active) human pooled serum (serum), heat-inactivated pooled human serum (HI-serum), or human pooled lipoprotein-depleted serum with LDL (+LDL). Mixes were immediately overlaid onto PC3 cells. Infections were carried out at an MOI of 0.1 for WT-G and 3.0 for the retargeted viruses, with final serum concentrations of 25% and LDL concentrations of 150 mg/dL. After 24 hours (WT-G) or 42 hours (retargeted viruses), cells were imaged using a Celigo Imaging Cytometer (FIG. 23). Data indicated that retargeted viruses are resistant to serum/LDL inhibition.

### Example 23. Cytoplasmic Tail Truncation of FLAV-G Enhances its Fusion Activity

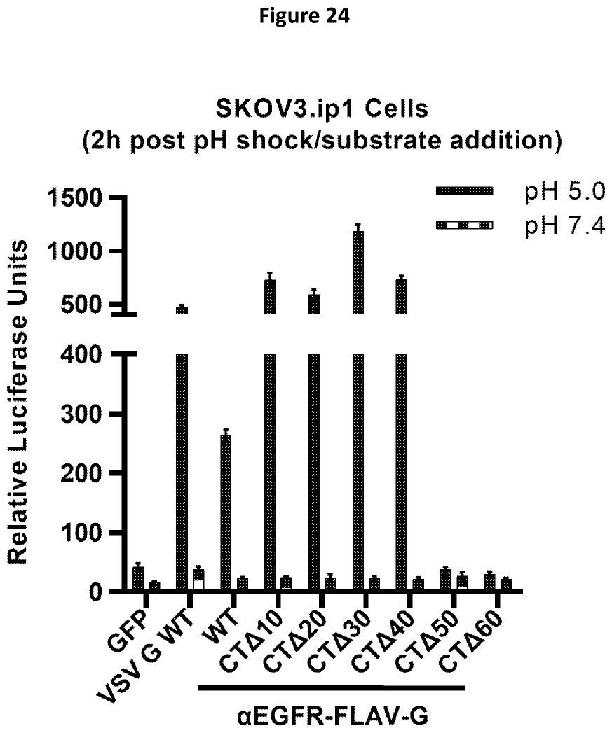

The present Example investigated the effects of cytoplasmic tail truncation on FLAV-G enhances fusion activity. For the experiment, a panel of FLAV-G constructs containing an anti-EGFR scFv were made with progressively shorter cytoplasmic tails. The fusion activity was determined by DSP cell-cell fusion activity in SKOV3.ip1 cells. Data indicate that truncation of 30 amino acids from the cytoplasmic tail provides optimal fusion activity (FIG. 24).

### Example 24. Improved Targeting of VSV to the HER2 Receptor in PC3 Cells

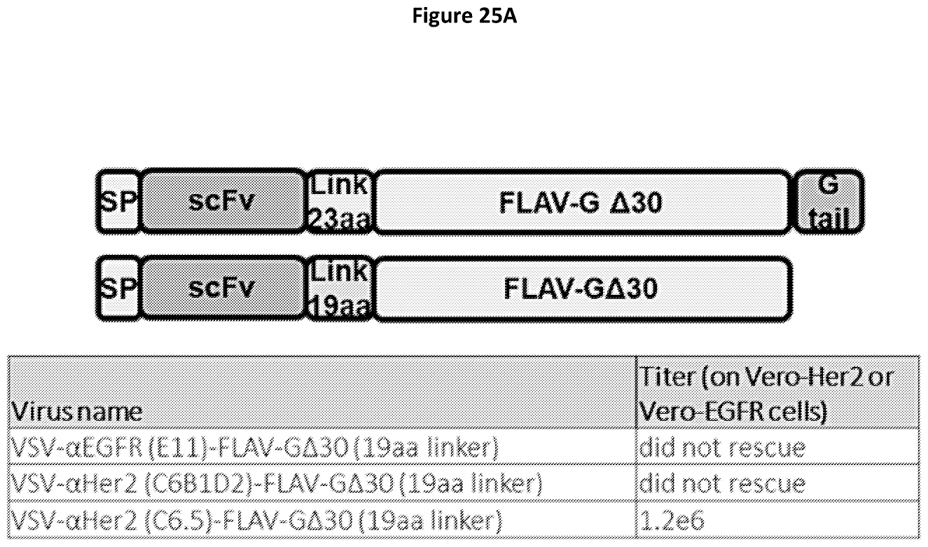

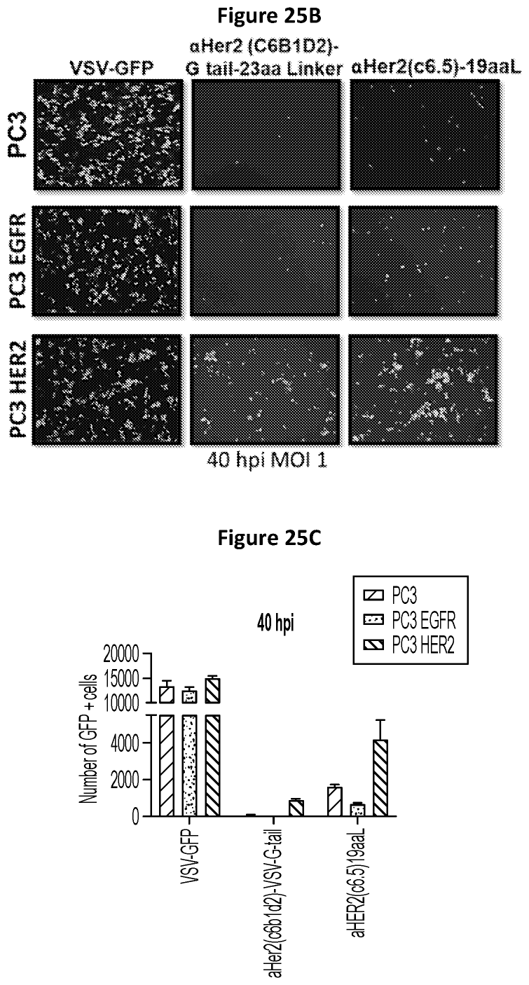

The present Example was designed to determine approaches for improved targeting of VSV constructs to the HER2 receptor in PC3 cells. Cartoon depictions of a truncated FLAV-G (FLAV-GA30) construct comprising a 23 aa linker and a VSV-G cytoplasmic tail compared to a construct comprising a 19 aa linker but lacking the VSV-G cytoplasmic tail is shown in FIG. 25A. Two scFvs targeting HER2 (C6B1D2 and C6.5) and one targeting EGFR were cloned into constructs comprising the 19 aa linker but only the C6.5 anti-HER2 construct was rescued (FIG. 25A, bottom). The specificity of the two HER2-targeted viruses (C6B1D2 and C6.5) were assessed on a panel of PC3 cells including parental PC3 cells which do not express HER2 or EGFR, PC3-EGFR cells, and PC3-HER2 cells. Infections were performed at an MOI of 1.0 (FIGS. 25B-25C). Fluorescence microscopy images of infected cells were acquired at 40 hours post infection using a Nikon microscope (FIG. 25B). A Celigo Imaging Cytometer was used to quantify the number of infected GFP-positive cells in each condition (FIG. 25C). Data indicated that a 19 aa linker combined with a lower affinity anti-HER2 scFv (C6.5) improves targeting to the HER2 receptor in PC3 cells.

### Example 25. FLAV G Targeted to Insulin-Like Growth Factor 1 Receptor

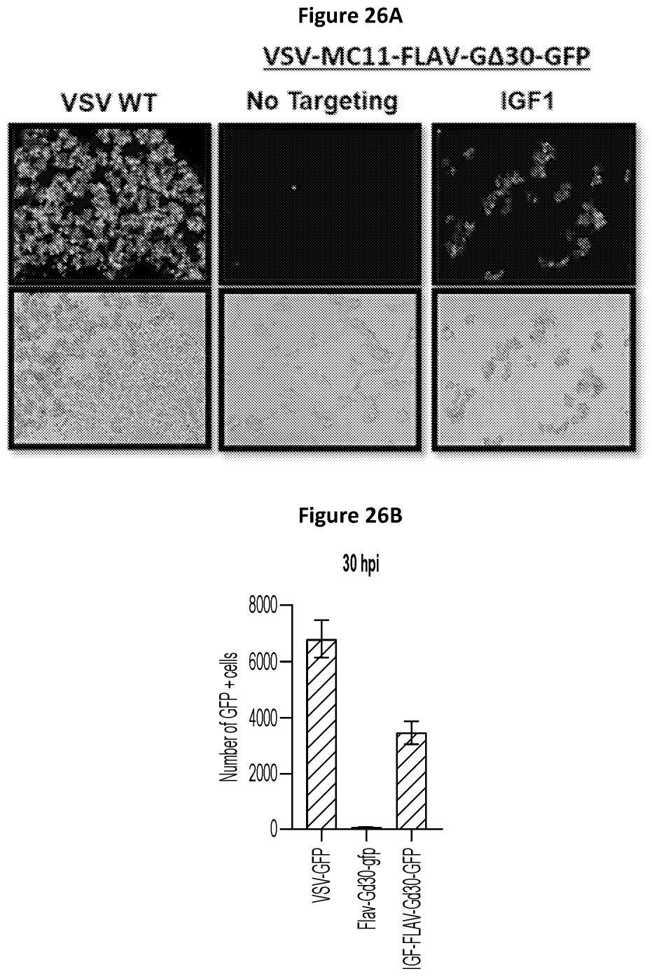

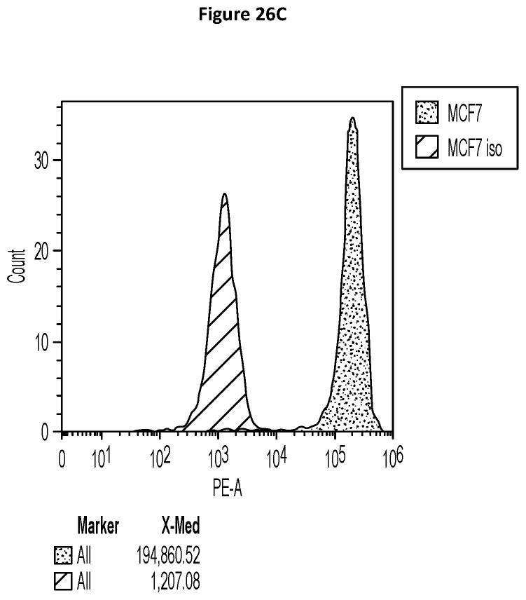

This Example tested FLAV-G designed to target insulin-like growth factor 1 (IGF1) receptor. For the experiments, metastatic adenocarcinoma epithelial MCF7 cells which express the IGF1 receptor were infected at an MOI of 1.0 with VSV-GFP (WT) or VSV containing a Flanders virus glycoprotein (FLAV-GA30, also termed FLAV-Gd30) that was free from any targeting molecule (No Targeting) or targeted to the IGF1 receptor with insulin like growth factor 1 (IGF1). Infected cells were imaged by fluorescent and phase contrast microscopy (FIGS. 26A-26B). Representative images of each condition were acquired 30 hpi using a Nikon microscope (FIG. 26A). The infected cultures were also imaged using a Celigo Imaging Cytometer and the number of GFP-positive cells per well of a 96-well plate was quantified (FIG. 26B). Flow cytometry was used to determine the expression of the IGF1 receptor in cells that had been stained with a fluorescently labeled anti-IGF1R antibody or an isotype control (FIG. 26C). Data indicated that FLAV-G can be targeted to infect IGF1 receptor-expressing cells in the context of a FLAV-G-expressing VSV.

### Example 26. Specificity of FLAV-G Displaying Modified EGF

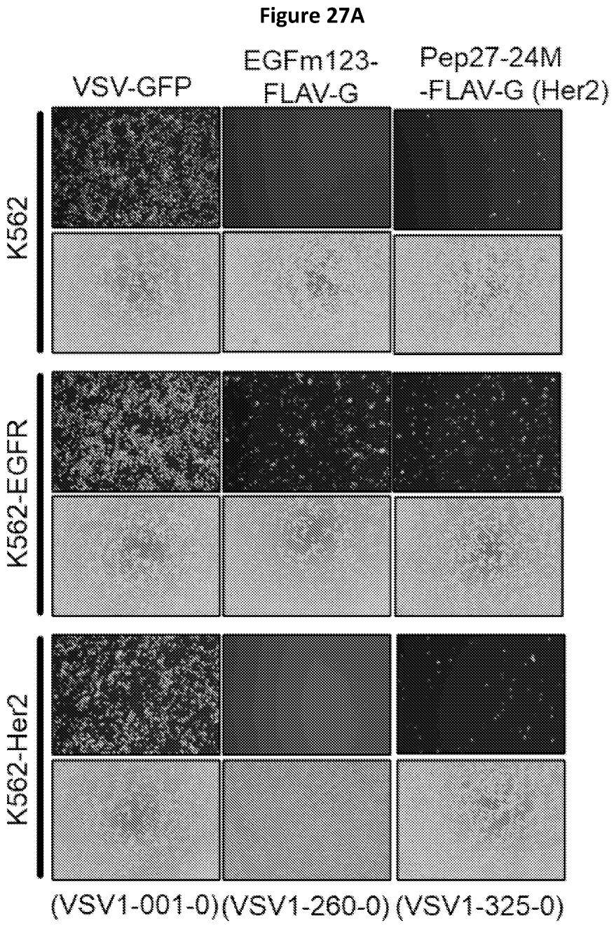

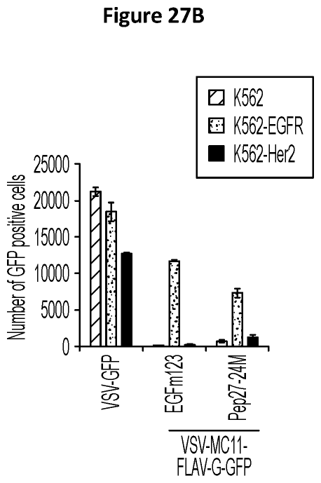

The Example was designed to assess the sensitivity of FLAV-G displaying modified epidermal growth factor (EGF). K562 cells stably transduced with EGF receptor or HER2 were infected at a MOT of 1.0 with VSV-GFP (WT) or VSV containing a Flanders virus glycoprotein (FLAV-GA30) displaying modified EGF (EGFm123) or HER2 (Pep27-24M). Infected cells were imaged using fluorescent and phase contrast microscopy (FIG. 27A). The infected cell cultures were also imaged using a Celigo Imaging Cytometer and the number of GFP-positive cells per well of a 96-well plate was quantified (FIG. 27B). Data indicated that FLAV-G can be targeted to infect EGF receptor-expressing cells in the context of a FLAV-G-expressing VSV.

### Example 27. Design of Retargeted VSV-G for Lentivirus Production

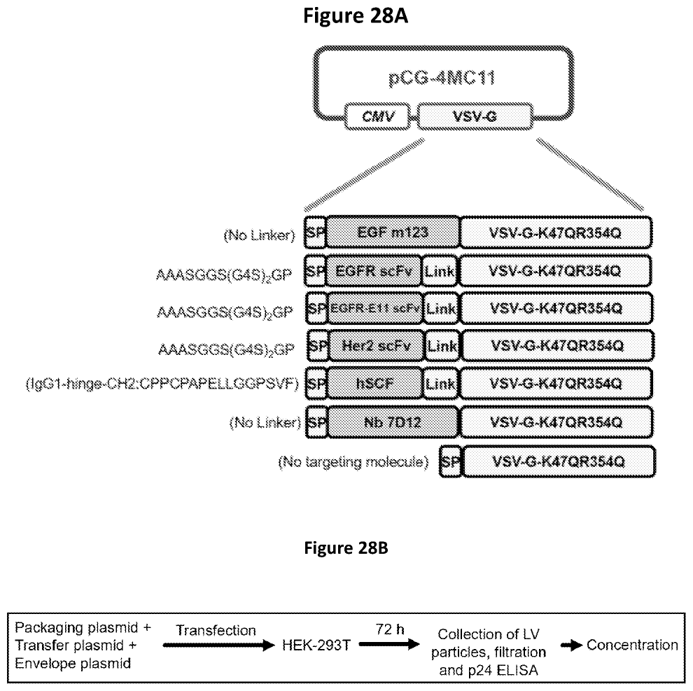

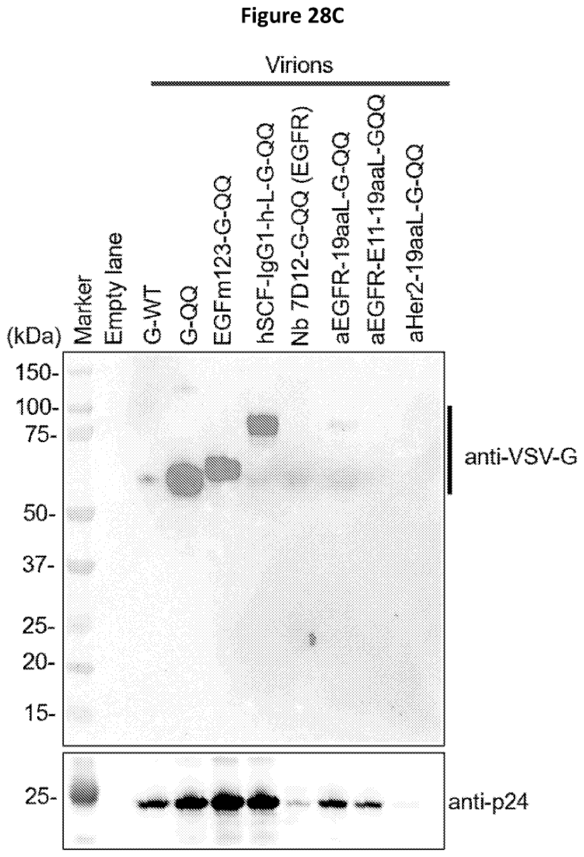

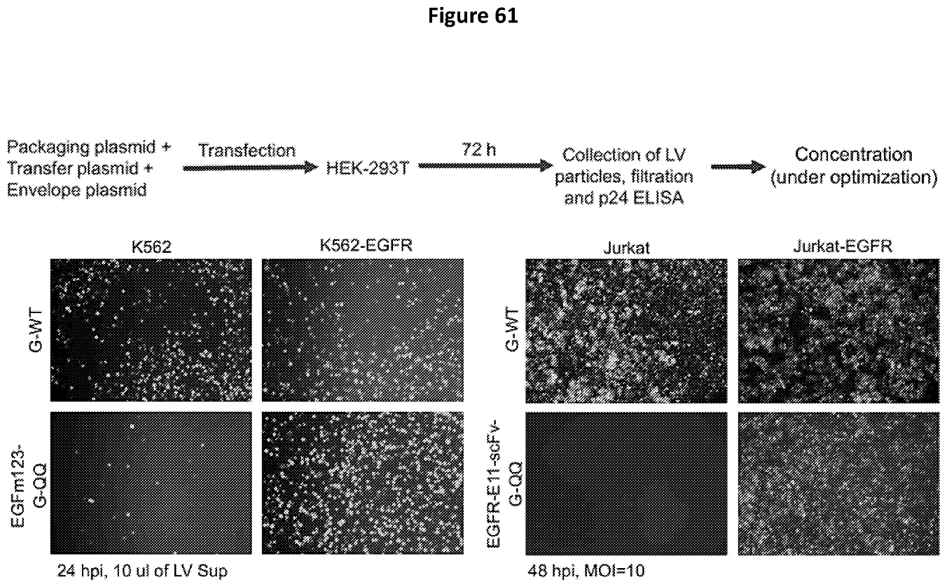

This Example describes the design of retargeted VSV-G for lentivirus production. A schematic of constructs generated are shown in FIG. 28A. Specifically, the VSV-G protein contained a signal peptide (SP) that was proteolytically cleaved following translation. The targeting molecule, for example, a scFv antibody raised against HER2 or EGF receptor, natural ligands such as EGFm123 (modified EGF) or hSCF, or nanobody, e.g., Nb 7D12 targeting EGF receptor, were appended to the amino-terminus of the G-protein with or without a flexible linker. A construct lacking a targeting molecule was generated as a control. Blinding mutations in VSV-G were incorporated at K47 and R354. A schematic of lentivirus production in HEK-293T cells is shown in FIG. 28B (but see also, e.g., FIG. 61, top panel). Three-days post-transfection in the HEK-293T cells, 1 ml of supernatant containing the collected lentiviral vectors was centrifuged at 13,000 rpm at 4° C. for 3 hours. The virus pellet was lysed and subjected to Western blotting analysis with anti-VSV-G and anti-p24 (lentiviral) antibodies (FIG. 28C). Proof-of-concept data supporting retargeting of G-pseudotyped lentiviral vectors via displayed domains including EGFm123 and anti-epidermal growth factor receptor (EGFR) scFv (E11) are also shown in FIG. 61.

### Example 28. Validation of Retargeted Lentiviruses on the Receptor-Positive Cell Lines

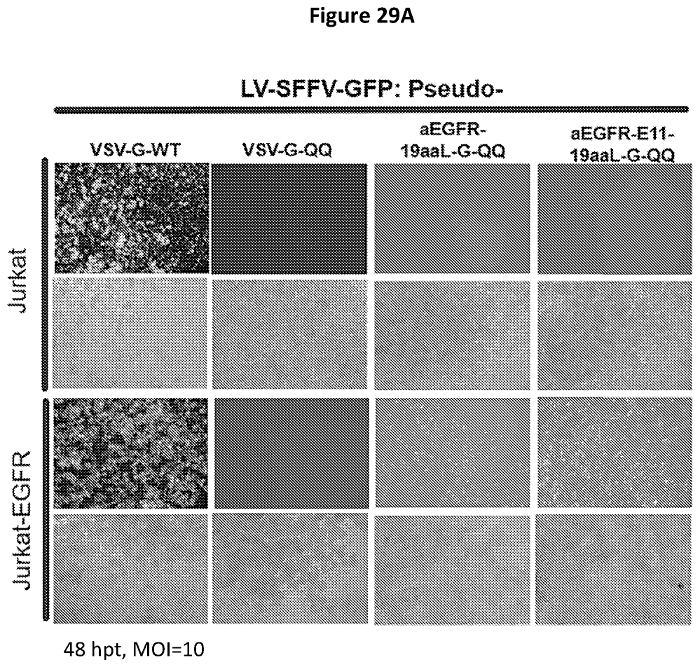

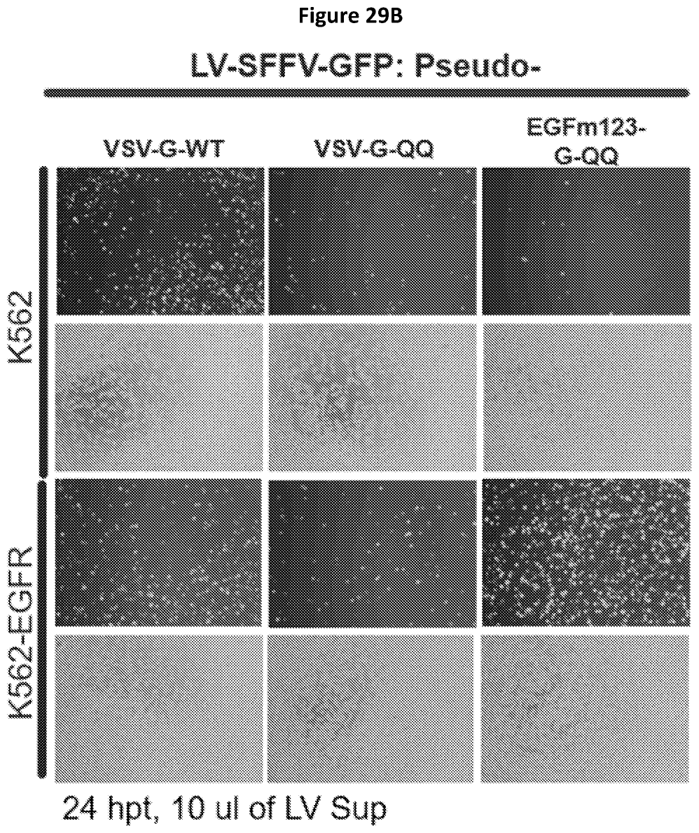

This Example was designed to validate retargeted lentiviruses using receptor-positive cell lines. To test the specificity of EGFR-targeted lentiviruses, lentiviruses pseudotyped with VSV-G-WT or VSV-G harboring K47Q/R354Q (G-QQ) mutations and appended to EGFR scFv or EGFR-E11 scFv or EGFm123 ligand were transduced into parental Jurkat or K562 cells which do not express EGFR, or a modified version of the cells that over-expressed EGFR. In Jurkat cells, the lentiviruses transduced with an MOI=10.0 and the fluorescence microscopy images were acquired at 48 hours post transfection (hpt) using a Nikon microscope (FIG. 29A). K562 cells were transduced with 10 μL of viral supernatant and fluorescence microscopy images were acquired at 24 hpt using a Nikon microscope (FIG. 29B). Data showed retargeting of the VSV-G-pseudotyped lentiviruses by introduction of the G-QQ mutation together with an EGFR-targeting molecule.

### Example 29. EGFR-Retargeted Lentiviral Vector is Less Sensitive to Serum and LDL Inhibition

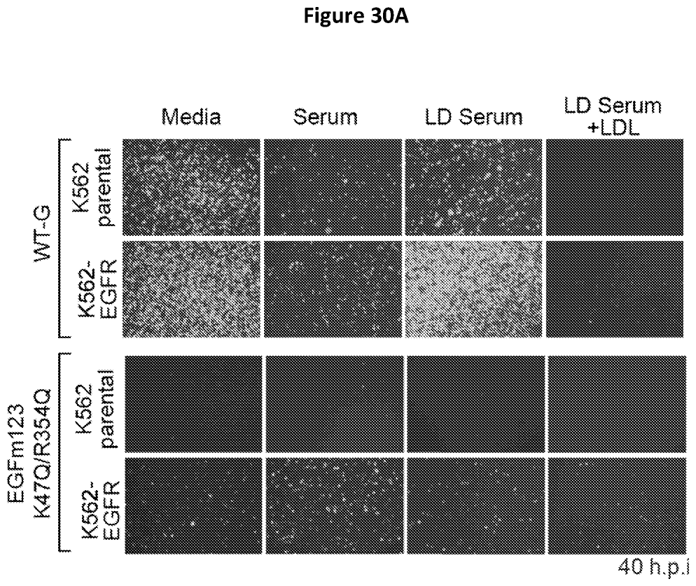

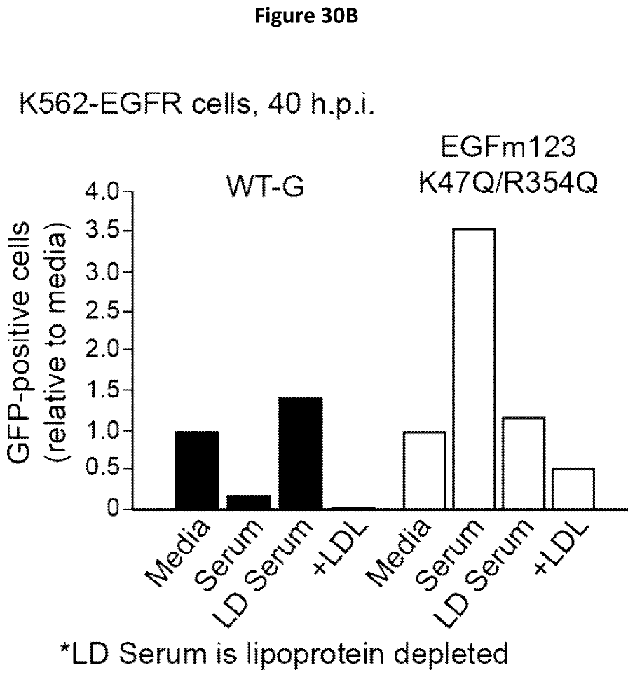

This Example tested the sensitivity of EGFR-retargeted lentiviral vectors to serum and LDL inhibition. A set volume (50 μL/well) of GFP-expressing lentiviral vectors pseudotyped with WT VSV-G (WT-G) or VSV-G harboring the K47Q/R354Q mutation and appended to EGFm123 (EGFm123 K47Q/R354Q) were used to transduce either K562 parental cells which do not express EGFR or modified EGFR-overexpressing K562 cells (K562-EGFR). Transductions were carried out in the presence of media alone, pooled human serum (complement deficient), pooled lipoprotein-depleted human serum, or pooled lipoprotein-depleted human serum and LDL. Serum concentrations were 25% and LDL concentration was 150 mg/dL. After 40 hours, cells were imaged by both fluorescence microscopy with an Olympus microscope (FIG. 30A) and Celigo Imaging Cytometer. The number of GFP-positive cells per well was determined using the Celigo software (FIG. 30B). Data indicated that EGFR-retargeted lentiviruses are resistant to inhibition by human serum.

### Example 30. VSV-G H80/K470/Y2090/R3540 Mutations Enhances the Specificity of Retargeted VSV Displaying EGFm123

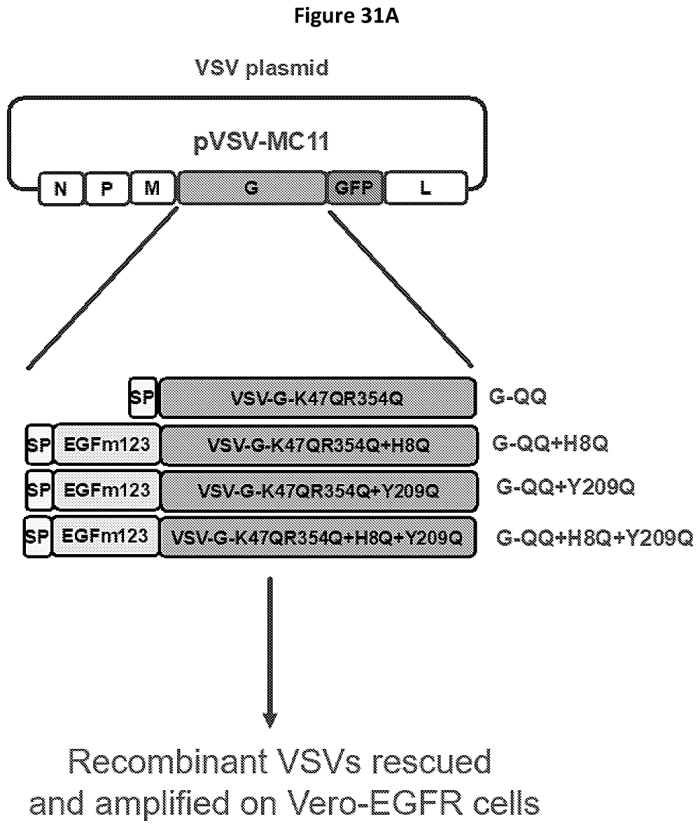

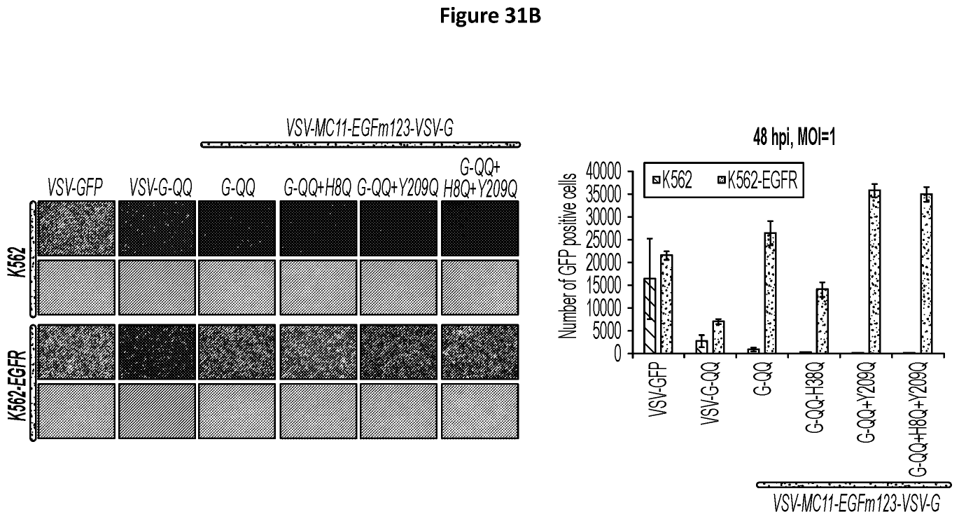

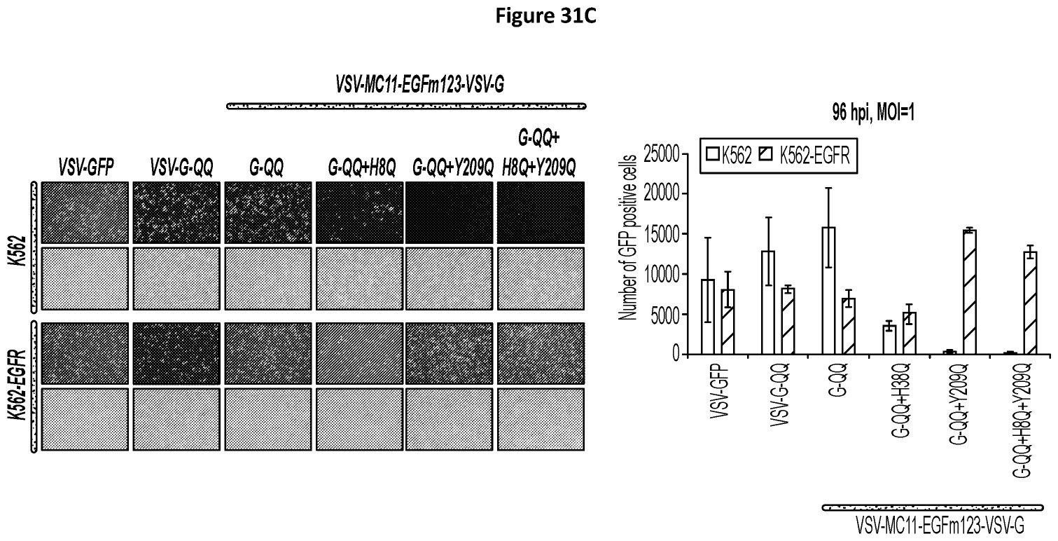

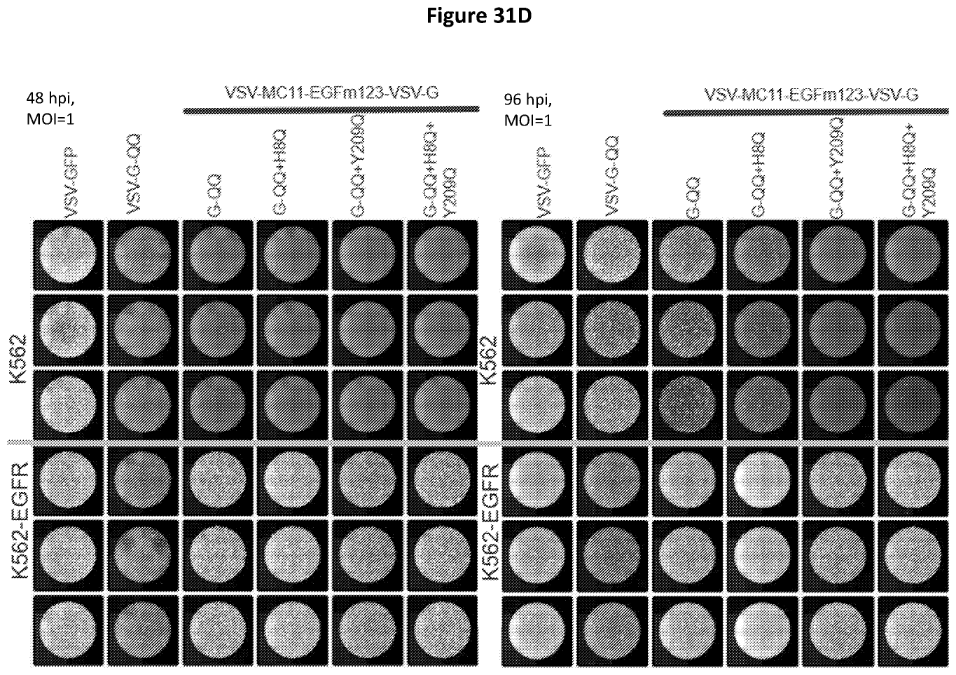

This Example investigated the effects of VSV-G H8Q/K47Q/Y209Q/R354Q mutations on the specificity of retargeted VSV displaying EGFm123. Designs of constructs comprising VSV-G H8Q/K47Q/Y209Q/R354Q mutations used in the present Example are shown in FIG. 31A. To test VSV-G H8Q/K47Q/Y209Q/R354Q mutations in retargeted VSV displaying EGFm123, K562 parental cells or K562-EGFR cells were infected with the VSV-EGFm123 viruses at an MOI of 1.0. Fluorescence microscopy images were acquired at 48 hpi (FIG. 31B) and 96 hpi (FIG. 31C) with 10× objective lens (left panels). Quantification of GFP-positive cells by a Celigo Imaging Cytometer is shown in FIGS. 31B-31C, right panels. Examples of GFP images captured by Celigo Imaging Cytometer are shown in FIG. 31D. These results indicated that combining the Y209Q mutation with VSV-G K47Q/R354Q enhanced the specificity of retargeted VSV displaying EGFm123, while combining the H8Q mutation had minimal or no effect on the enhancement of retargeting specificity.

### Example 31. Deletion of K47 Residue in the VSV-G Ablates the VSV Tropism and is Redirected to EGF Receptor Positive Cells

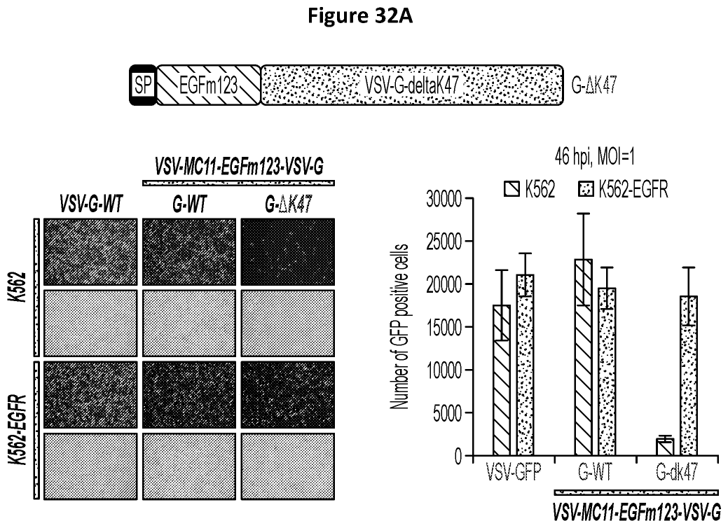

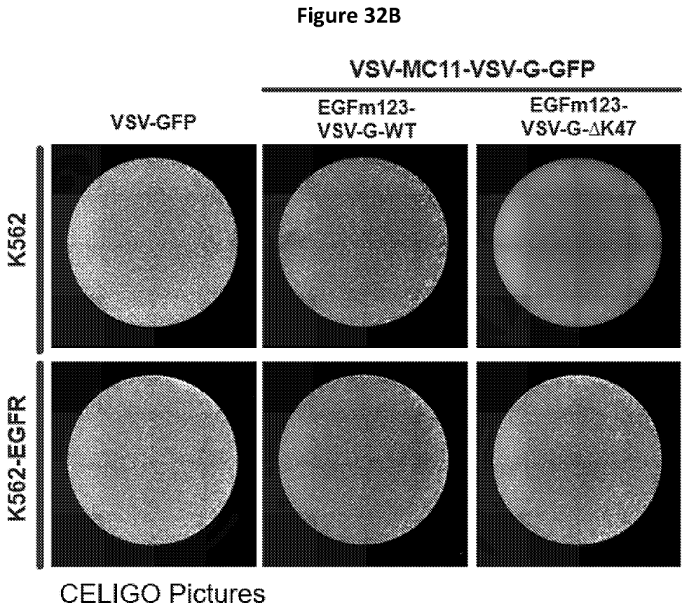

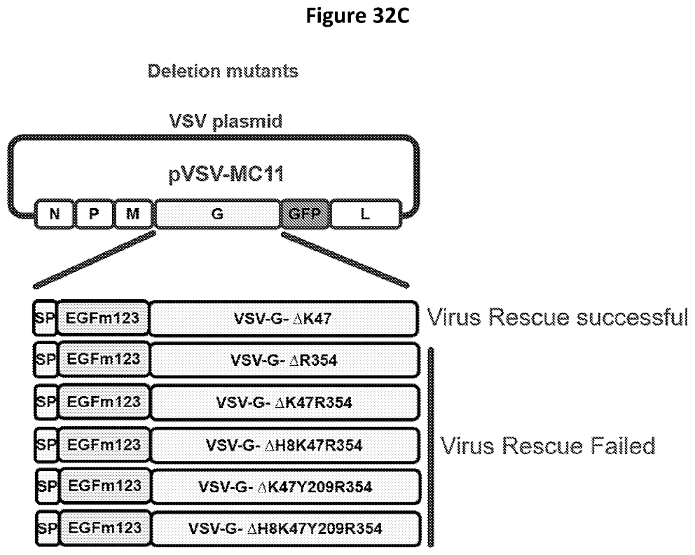

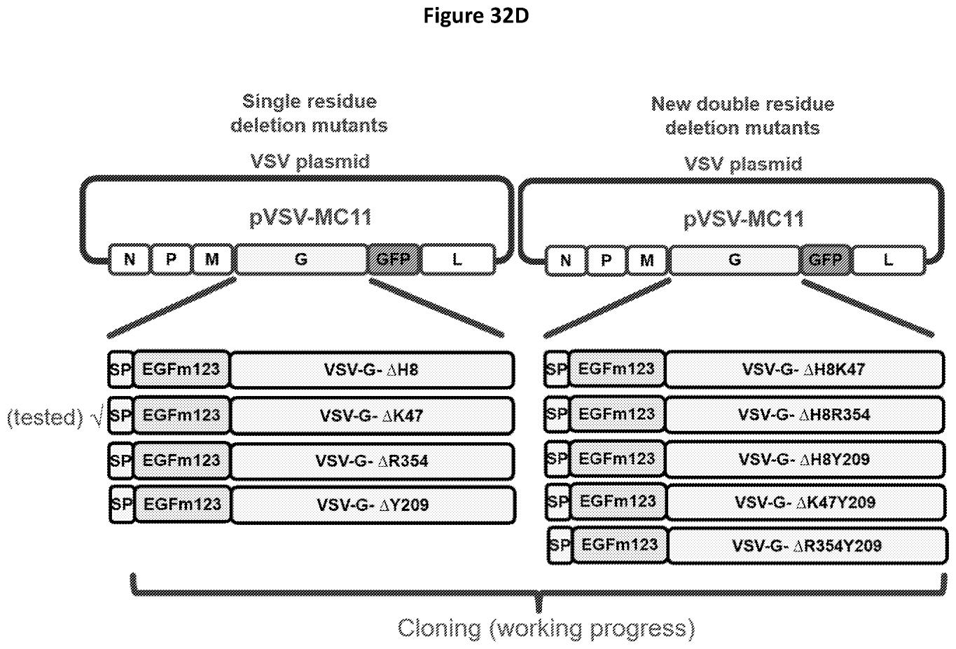

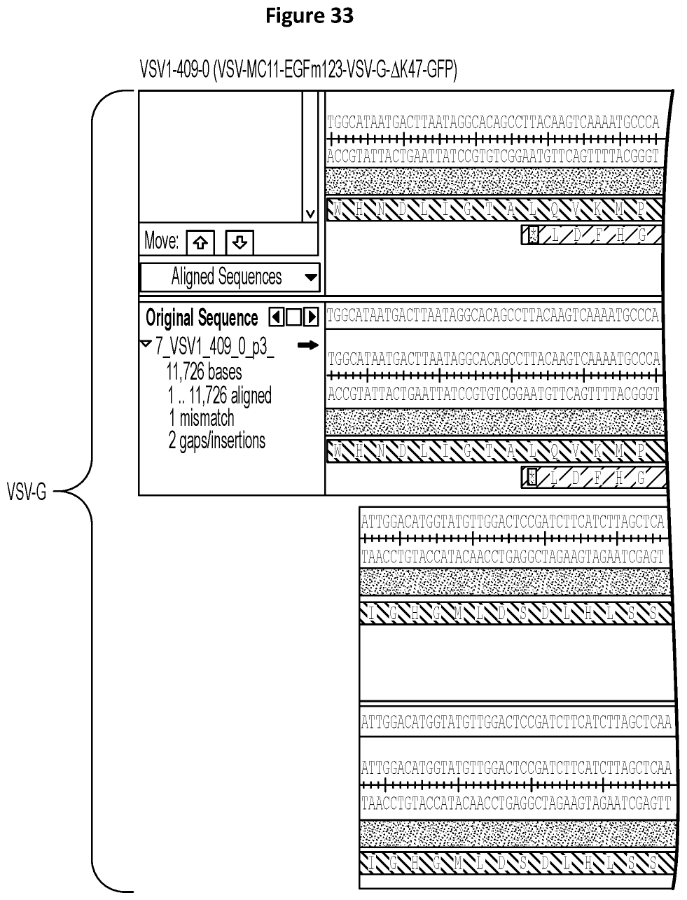

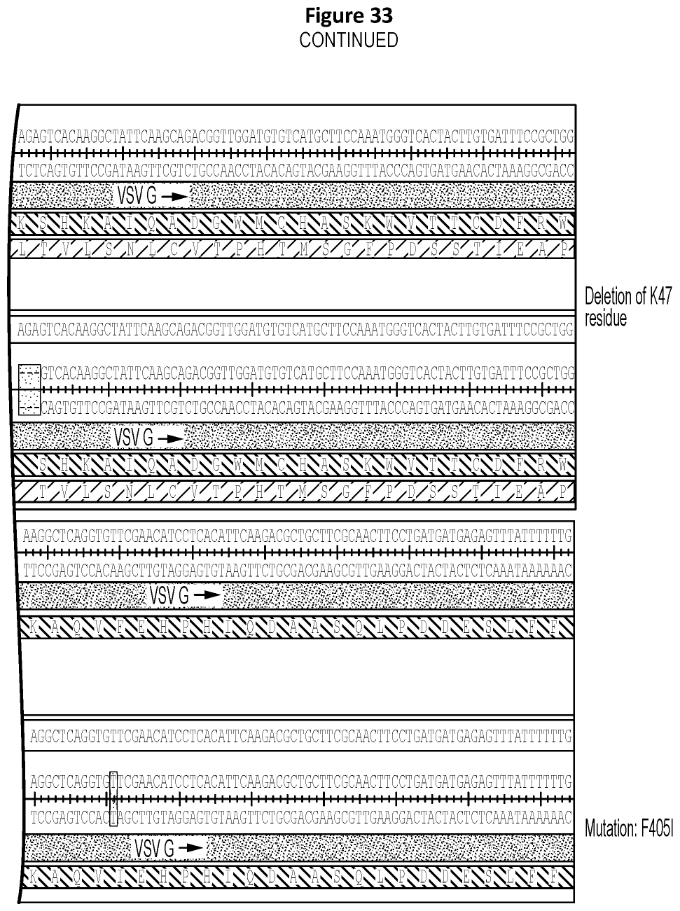

This Example tested the effects of deletion of the VSV-G K47 residue (G-ΔK47) on VSV tropism and redirection of VSV-EGFm123 to EGF receptor positive cells. A description of amino acid and nucleotide sequences of an example VSV-EGFm123 construct comprising deletion of the K47 residue and a point mutation at F405I is shown in FIG. 33. For the experiments, K562 parental cells or K562-EGFR cells were infected with VSV-GFP or VSV-EGFm123 displaying G-WT or G-ΔK47 VSVs at an MOI of 1.0. A cartoon schematic of the VSV-EGFm123 displaying G-ΔK47 is shown in FIG. 32A (top panel). Fluorescence microscopy images were taken at 46 hpi with 10× objective lens (FIG. 32A, bottom left panel). Quantification of the GFP-positive cells by a Celigo Imaging Cytometer is shown in the bottom right panel of FIG. 32A. Examples of GFP images captured by the imaging cytometer are shown in FIG. 32B. Data indicated that the deletion of K47 residue can ablate VSV-G binding to LDLR. Additional examples of constructs comprising the deletion of the VSV-G K47 residue and further comprising deletions of H8, Y209 and/or R354 residues is shown in FIG. 32C. Additional examples of double residue deletion mutations (e.g., comprising deletions of H8 and K47 or R354 or Y209 residues, or comprising deletions of Y209 and K47 or R354 residues) are described in FIG. 32D.

### Example 32. Targeting Alternate Rhabdoviral G Proteins

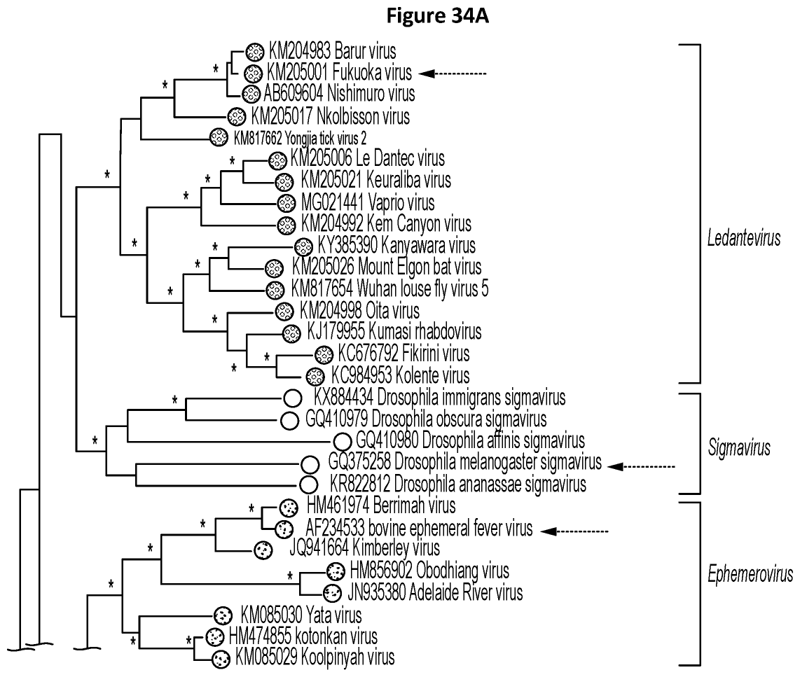

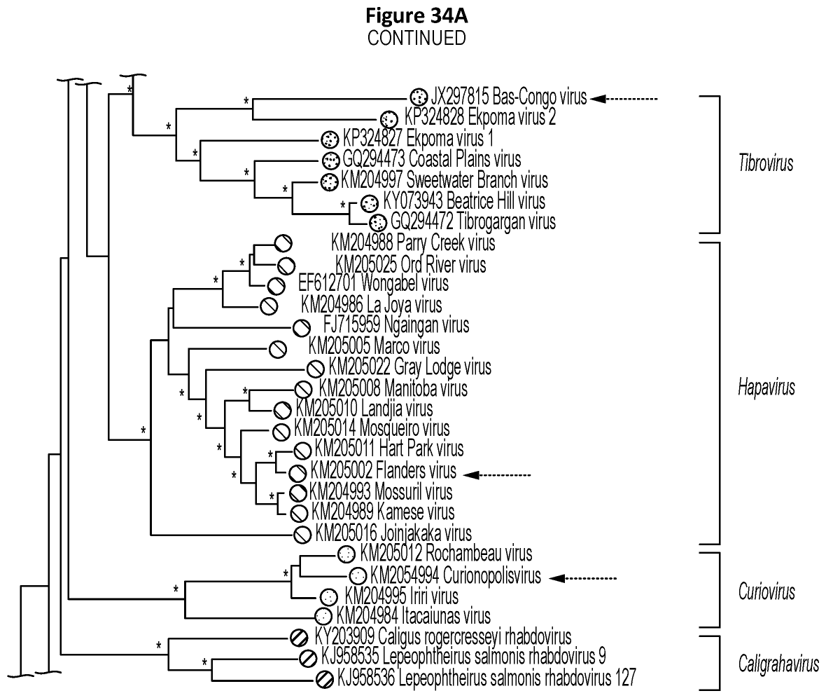

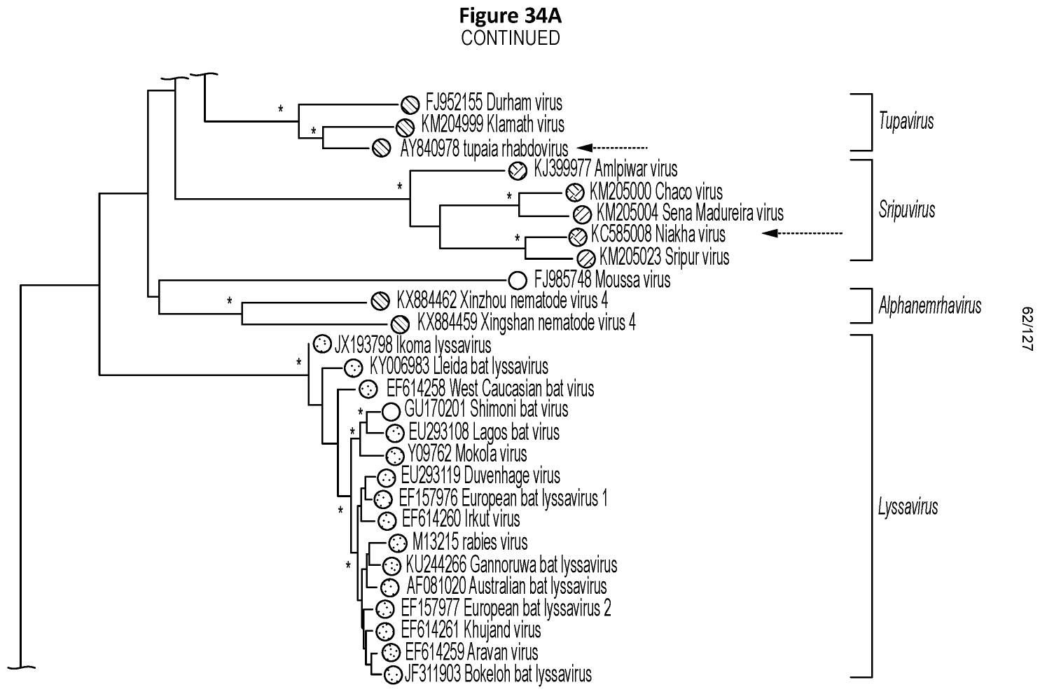

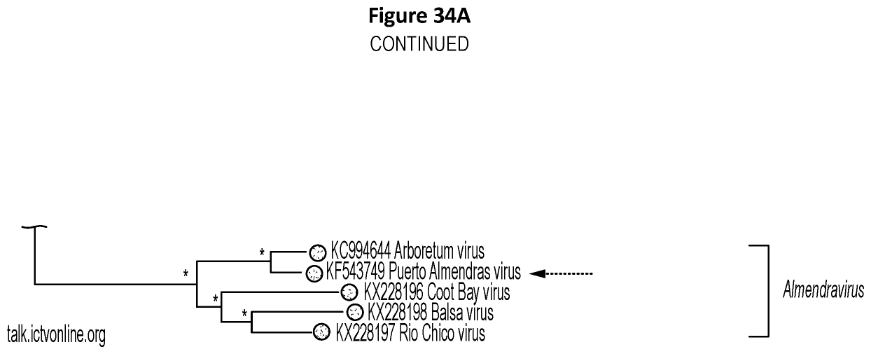

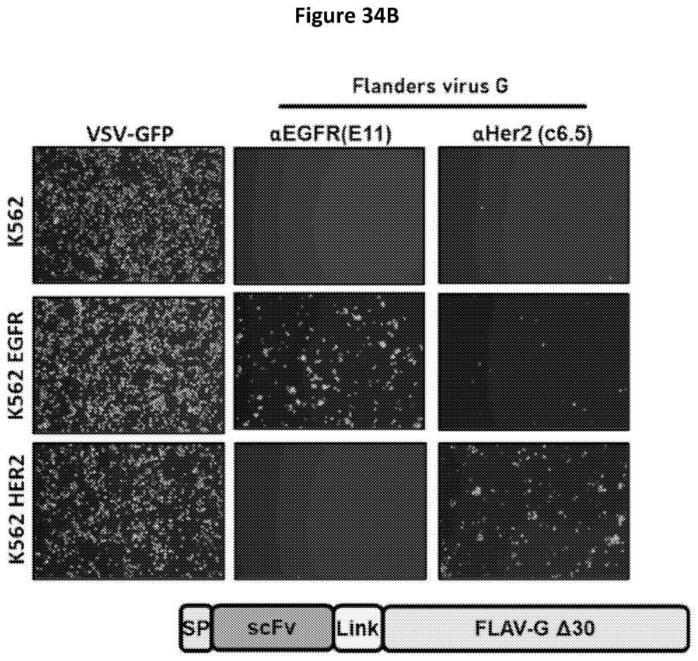

This Example investigated the targeting of alternate rhabdoviral G proteins. Specifically, nine glycoproteins were selected from nine different rhabdovirus genera (FIG. 34A). As an example, glycoproteins were selected from viruses which did not infect human cells or infected human cells at a narrower range than VSV. Rhabdoviruses that infected plants were also excluded. Further excluded were other vesiculoviruses which may use LDLR and/or could have broad tropism, as well as Lyssaviruses due to neurotropism, e.g., as in Rabies virus. Nucleotide sequences of the selected G proteins were identified in the UniProt database then were optimized for human codon usage using the JCat codon optimization tool. The resultant genes were artificially synthesized and subcloned into a plasmid backbone. SignalP 5.0 was used to predict the signal peptide of each G protein. Genes encoding scFvs and a linker were inserted following the predicted signal peptide sequence. Following screening assays described herein, Flanders virus (FLAV) G (FLAV-G) was selected as a lead for targeting. Screening of different length constructs showed that the optional length of the cytoplasmic tail for the FLAV G-protein was A30. VSVs containing a GFP reporter gene and FLAV-GA30 with scFvs targeting EGFR or HER2 were used to infect a panel of K562 cells that stably expressed EGFR or HER2. The cells were imaged using a fluorescence microscope (FIG. 34B).

### Example 33. Screening of Additional Alternate G Proteins

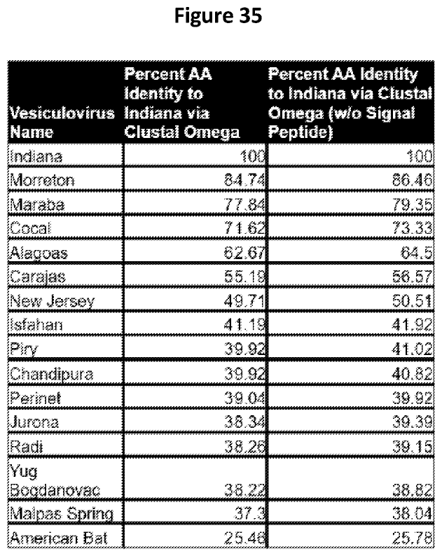

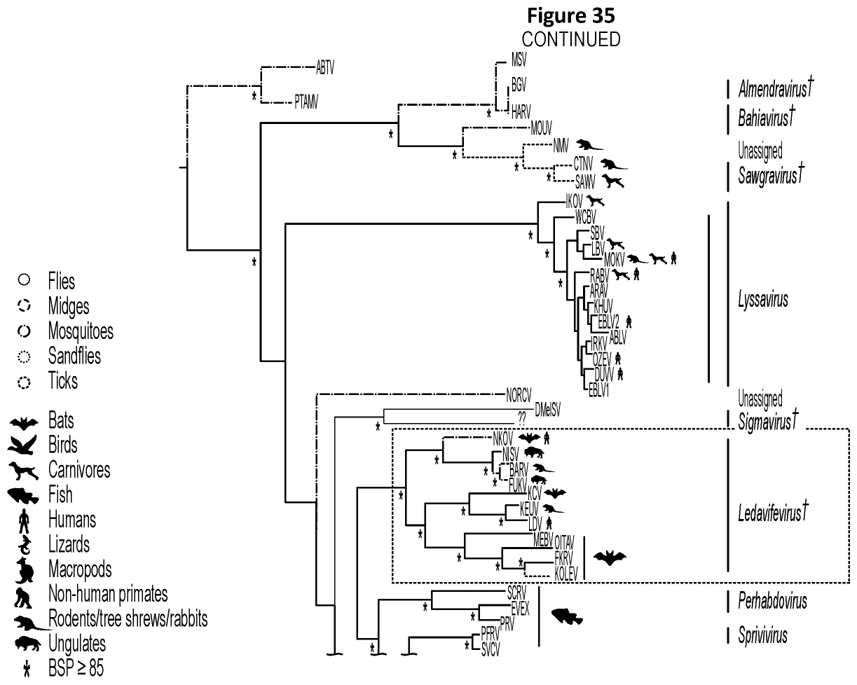

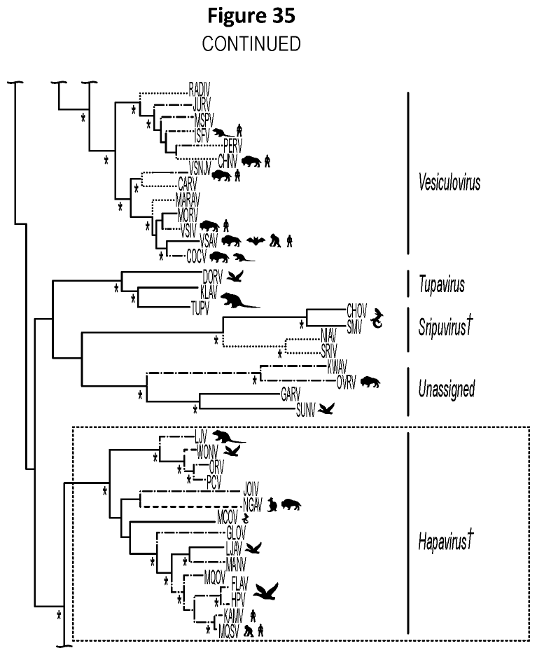

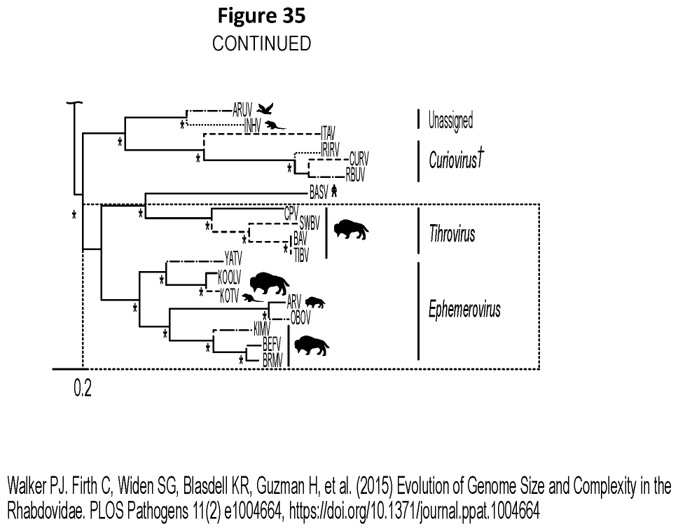

This Example involved screening of additional alternate G proteins. Twenty additional viral glycoproteins were selected for screening to identify glycoproteins that may be retargeted to receptors of interest. Virus species in the vesiculovirus genus of Rhabdoviridae are described in the table shown in FIG. 35 (left panel). The percent amino acid identity to vesicular stomatitis Indiana virus (VSIV) was computed using Clustal Omega with or without the signal peptide sequence included. The vesiculoviruses with approximately 70% amino acid sequence identity to VSIV, e.g., as set forth in SEQ ID NO: 8, were excluded. Additional glycoproteins from the Ledantevirus, Hapavirus and Ephemerovirus genera were selected, namely Fukuoka (Ledantevirus), Flanders (Hapavirus), and Bovine ephemeral fever (Ephemerovirus) (FIG. 35, right panel). As an example, additional Ledantevirus glycoproteins were selected based on the broad tropism of Fukuoka virus G (FUKV-G) in a DSP fusion assay described herein. Additional Hapaviruses were selected based on the success of retargeting Flanders virus G (FLAV-G) to EGFR, HER2 and IGF1R (see, e.g., FIG. 24, and FIG. 26). Additional Ephemerovirus glycoproteins were included based on the narrower tropism of Bovine ephemeral fever G (BEFV-G) in human cells in a DSP assay, as well as the ability of BEFV-G to retain its fusion activity after insertion of an N-terminal 6× Histidine tag (SEQ ID NO: 235), and the high level of cell surface expression of BEFV-G even with an N-terminal 6× His tag (SEQ ID NO: 235).

### Example 34. Screening of Additional Non-VSV Rhabdoviral G Proteins

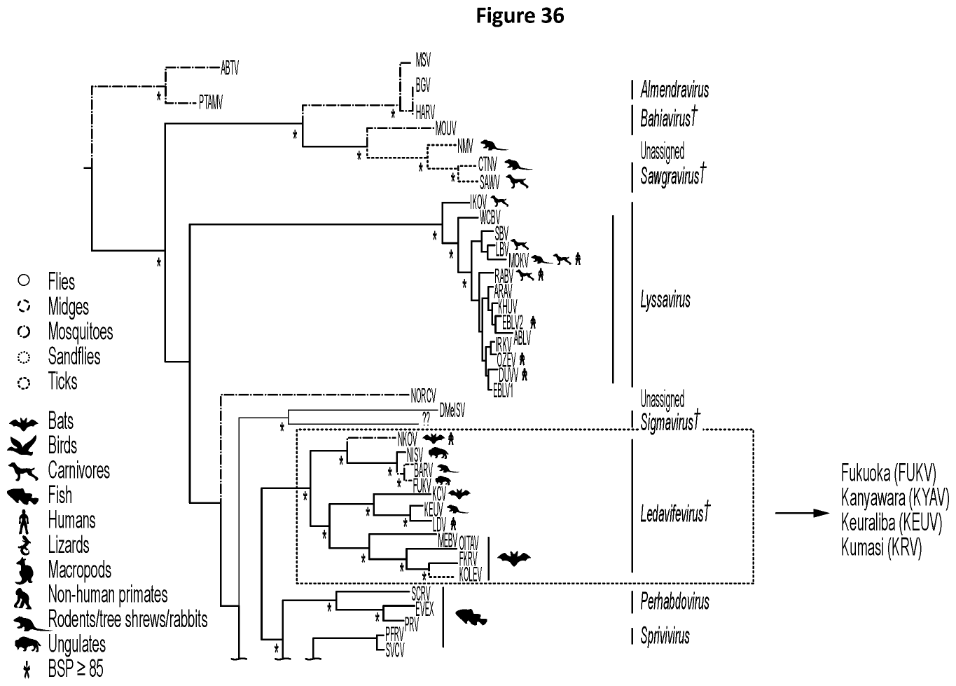

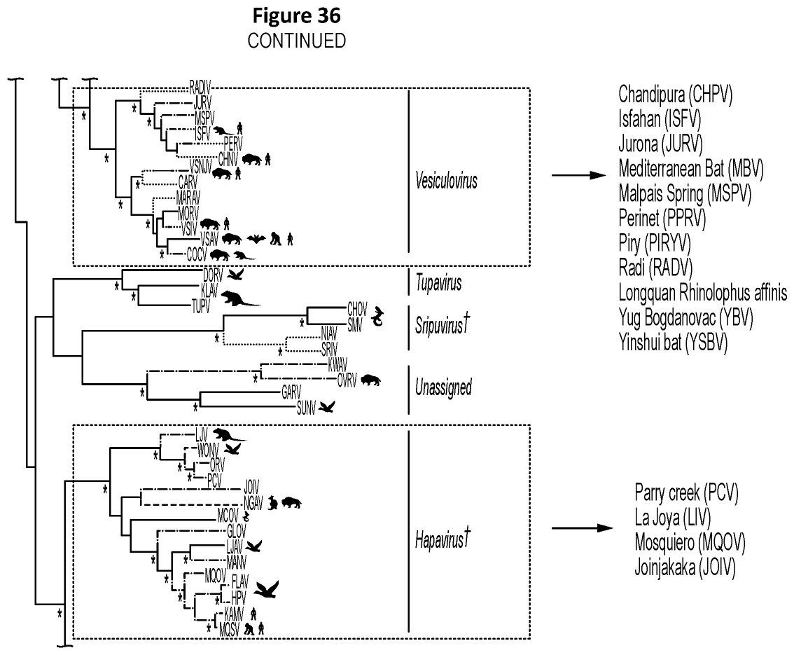

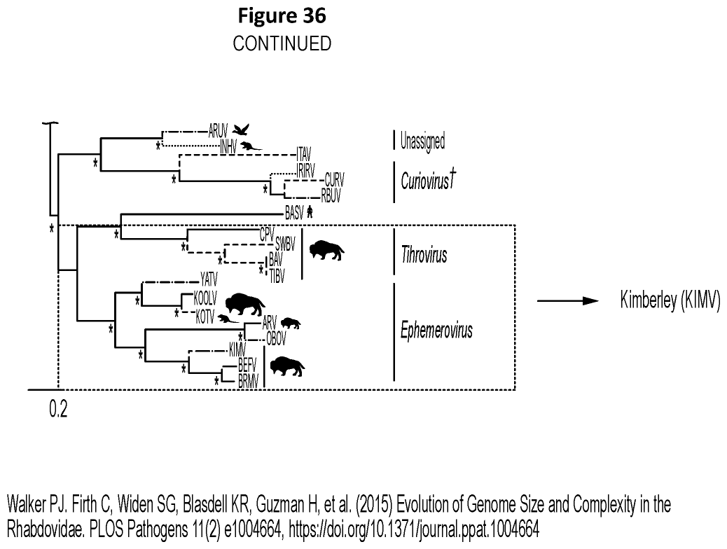

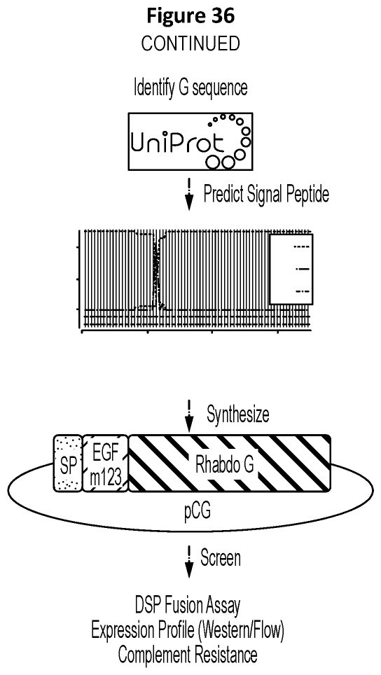

This Example describes an alternate format to that described above (see, e.g., Example 33) for the screening of additional non-VSV rhabdoviral G proteins. Twenty additional viral glycoproteins were selected for screening to identify glycoproteins that may be retargeted to receptors of interest and may be resistant to complement inactivation. Virus species in the vesiculovirus genus of Rhabdoviridae were selected based on having a percent amino acid sequence identity less than 70% with vesicular stomatitis Indiana virus. Additional glycoproteins from the Ledantevirus, Hapavirus and Ephemerovirus genera were selected, e.g., Fukuoka (Ledantevirus), Flanders (Hapavirus) and Bovine ephemeral fever (Ephemerovirus) virus glycoproteins, in the first phase of screening (FIG. 36, left panel). For each glycoprotein, the signal peptide sequence was predicted using SignalP 6.0 and a modified epidermal growth factor (EGFm123) was inserted immediately following the signal peptide sequence. These modified glycoprotein genes were synthesized and subcloned into a protein expression vector (pCG) (FIG. 36, right panel). Examples of amino acid sequences of non-VSV rhabdoviral G proteins are set for in SEQ ID NO: 9-15, 17, 60-79, 81-108.

### Example 35. Functional Screening of Alternate EGF-Displaying Rhabdoviral G Proteins for EGFR Targeting

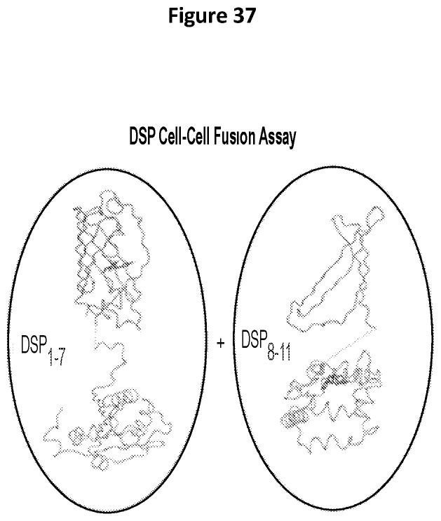

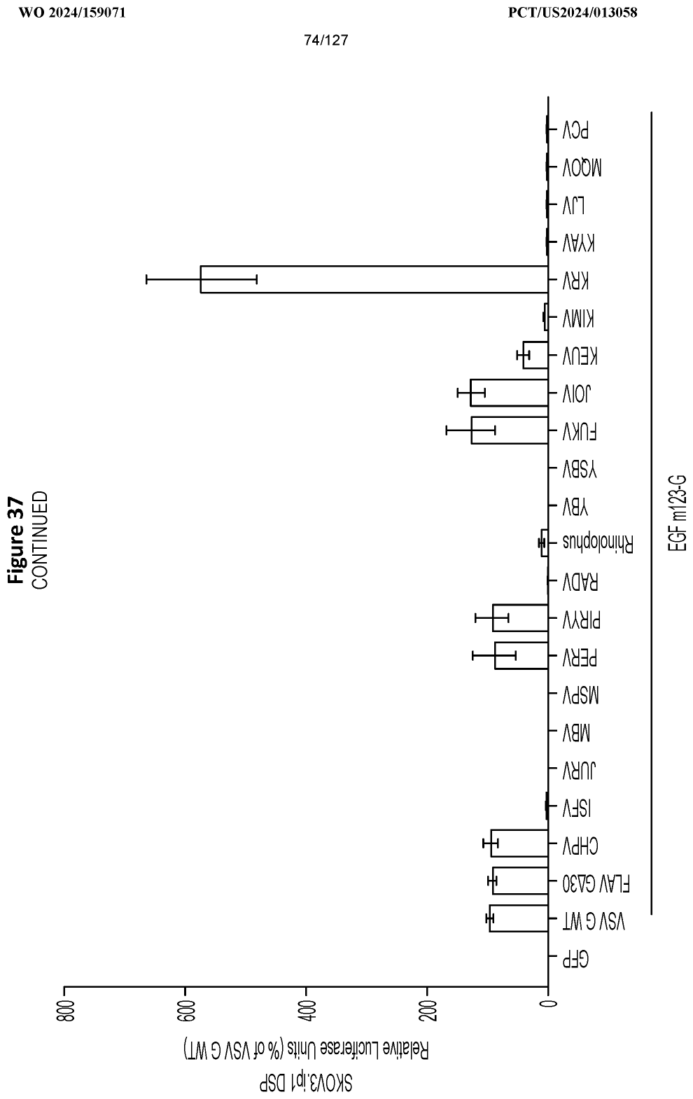

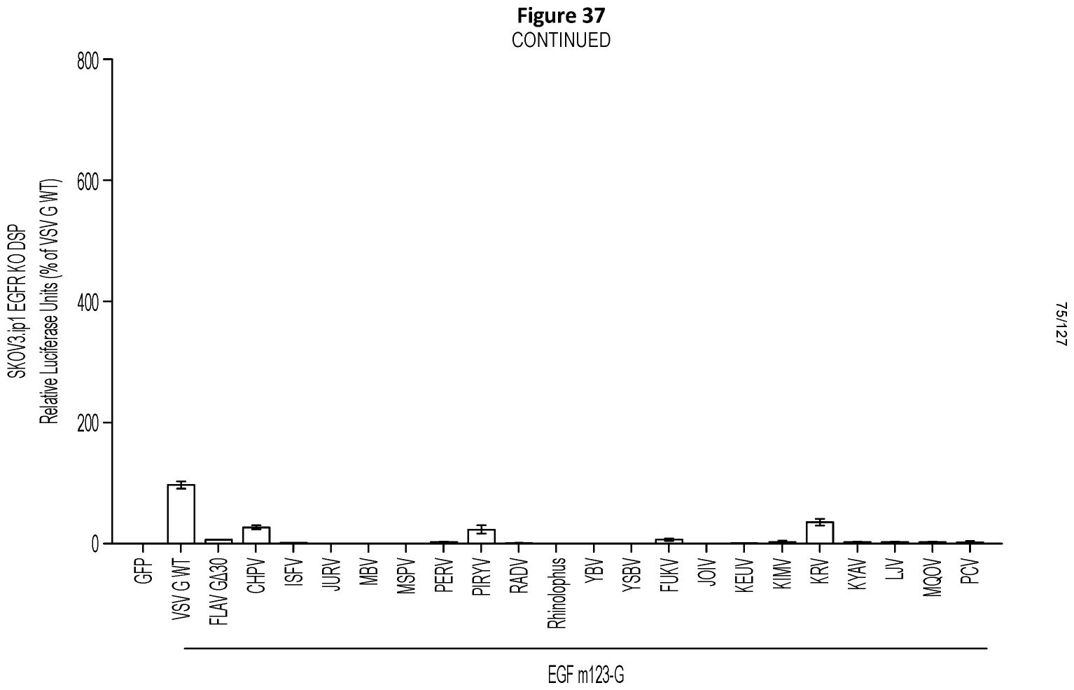

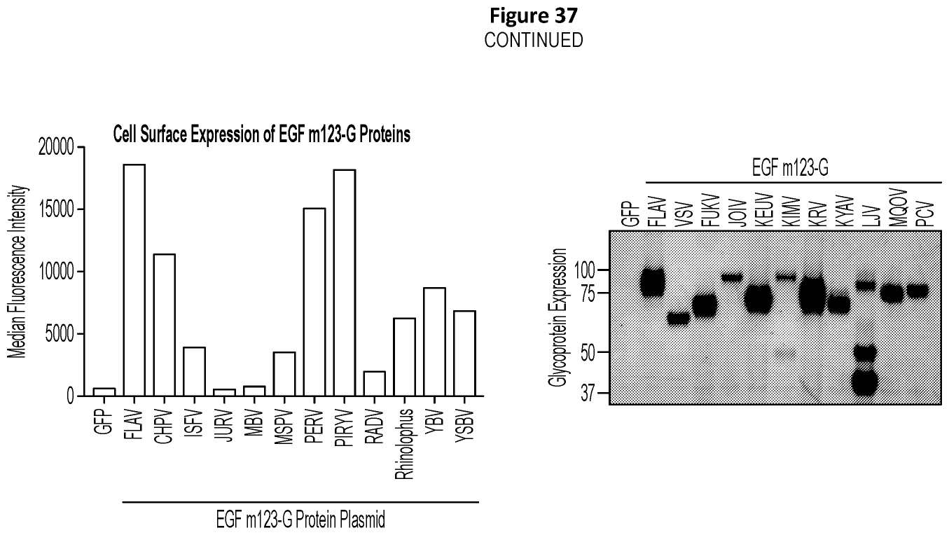

The present Example provides an example approach for the functional screening of alternate EGF-displaying rhabdoviral G proteins for EGFR targeting. To measure fusion activity of the various glycoproteins, a panel of SKOV3.ip1 cells was generated. Epidermal growth factor receptor (EGFR) knockout (KO) SKOV3.ip1 cells were generated using CRISPR/Cas9. Parental or EGFR KO cells were then transduced with one half of a dual-split protein (DSP) reporter gene that contained split GFP and Renilla luciferase (see, e.g., FIG. 37, left panel). Twenty viral glycoproteins targeted to EGFR via EGFm123 were transfected into a mixed pool of DSP1-7 and DSP8-11 cells (Parental or EGFR KO cells). A plasmid expressing GFP served a negative control for fusion, and non-targeted VSV-G WT and EGFR-targeted FLAV-GA30 served as positive controls for fusion. The day after transfection, the media was removed and replaced with either neutral pH or pH 5.0 PBS for 2 minutes. The PBS was then replaced with fresh media containing the EnduREN luciferase substrate and the luminescence was recorded 2 hours later (FIG. 37, middle panel). Cells surface expression of EGFm123-G proteins were additionally quantified using fluorescence microscopy and Western blotting approaches (FIG. 37, right panel).

### Example 36. Pseudotyping Lentivirus with Targeted G Proteins

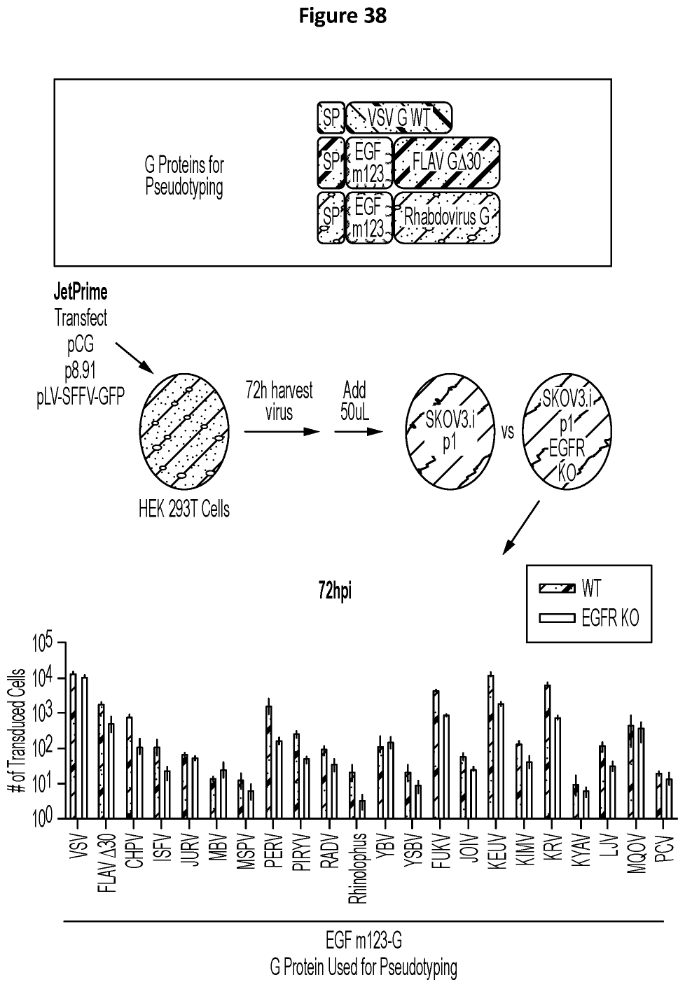

The present Example describes an example approach for pseudotyping of lentiviruses with targeted G proteins. For the experiment, lentiviruses pseudotyped with a panel of targeted rhabdovirus glycoproteins (or non-targeted VSV-G WT and EGFR-targeted FLAV-GA30 as controls) were generated by expressing the glycoprotein vector (pCG), packaging plasmid (p8.91) and the genome vector expressing GFP (pLV-SFFV-GFP) in HEK 293T cells (FIG. 38, left panel). Three days later the culture media containing the lentiviruses was harvested. 50 μL of the supernatant was then added to monolayers of SKOV3.ip1 or SKOV3.ip1 EGFR KO cells in a 96-well plate. The number of GFP-positive cells was determined using a Celigo Imaging Cytometer in the WT versus the EGFR KO cells (FIG. 38, left panel).

### Example 37. High-Throughput Single Chain Fragment Variable Library Screen by DSP Transfection Assay

Vesicular stomatitis virus (VSV) can be a useful viral antigen for the treatment of a variety of cancers and possesses a genetic flexibility to accept different payloads to enhance its potency. VSV utilizes the low-density lipoprotein receptor (LDLR) for virus entry into the cell, which is ubiquitously found throughout the human body (see, e.g., FIG. 8B). An example platform to retarget the VSV virus is engineering the VSV glycoprotein (VSV-G) to lack LDLR binding potential as described herein. This allows for retargeting of the viruses by attaching single chain fragment variables (scFvs) to VSV-G proteins to specific cancer antigens; however, selecting the correct scFv can be a challenge on its own. As such, the present Example describes the development of dual-split protein (DSP) assays and lentivirus screens to rapidly determine which scFvs from a library would be most suitable for incorporating into the virus.

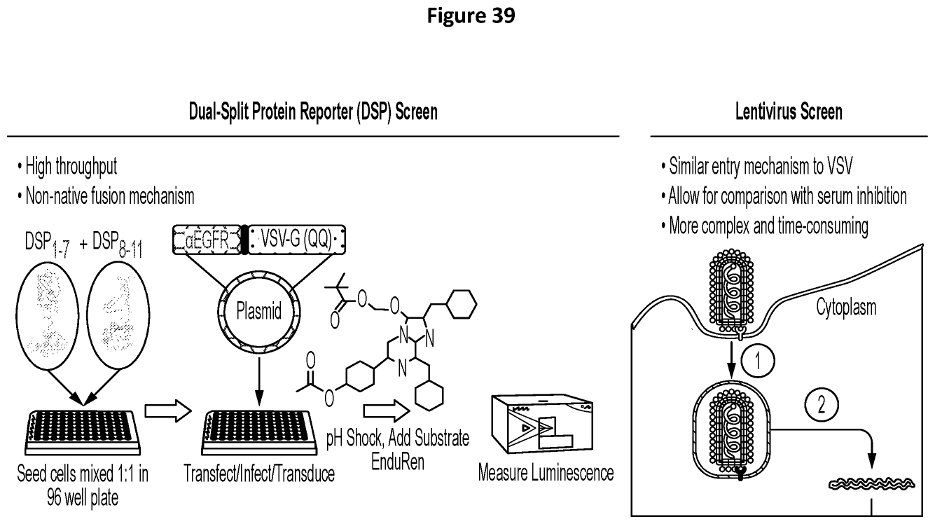

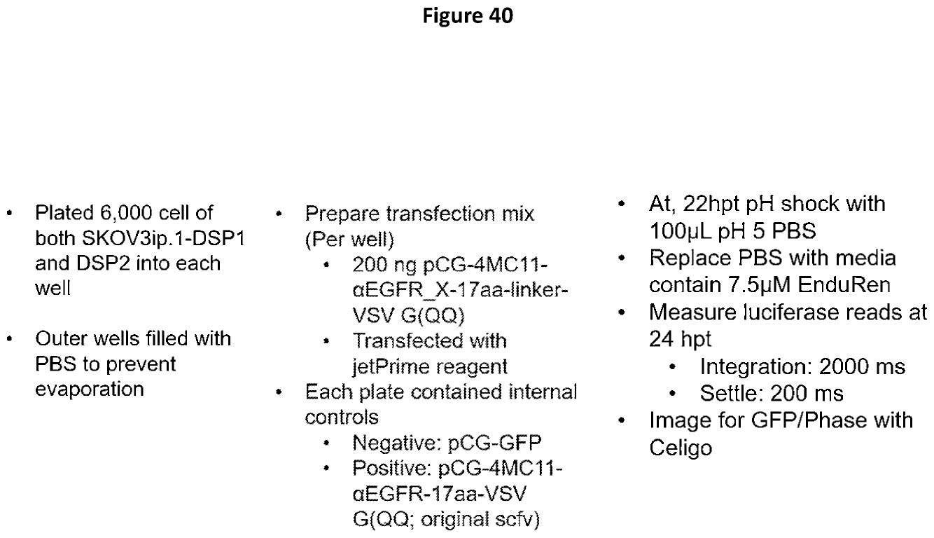

DSP assays consist of two chimeric proteins consisting of half the Renilla luciferase protein and half green fluorescent protein (GFP) on each protein, which must come together in order to form fully functional proteins to produce a signal (FIG. 39). In the assay described herein (see, e.g., FIG. 40), the chimeric proteins (DSP1 and DSP2) are separated by encoding the chimeric proteins into two different cells, which can then only form a functional unit when the two types of cells fuse together. Fusion can be achieved by, e.g., transfection with VSV-G and addition of acidic buffer but relies on the ability for the proteins to bind between the two cells. As such, the fusogenicity of the proteins can be quantified through luciferase readouts and, if the binding between cells is occurring through scFvs, candidates for retargeting VSV recombinants can be selected.

While the DSP assay allows for a fast and high-throughput method for screening scFvs, in the case of VSV-G the process can be forced since an acid buffer may be needed to drive fusion. In contrast, lentiviruses are produced using VSV-G and enter the cell using the same mechanisms as the VSV virus, making it a biologically relevant proxy for VSV infection. Screening with the different scFv requires replacing the WT VSV-G plasmid with one expressing a blinded VSV-G encoding a scFv sequence. Lentiviral titration can then be used as a functional readout to determine which sequences would be suitable for VSV rescue.

### Results

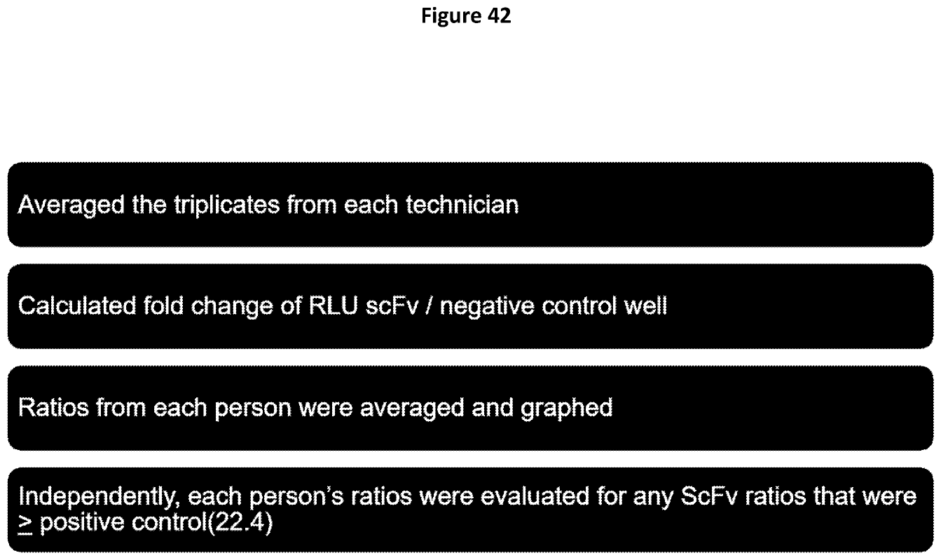

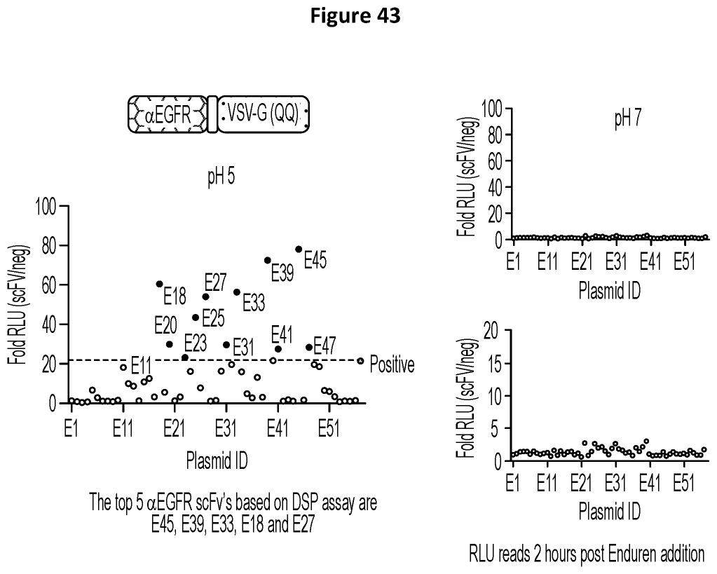

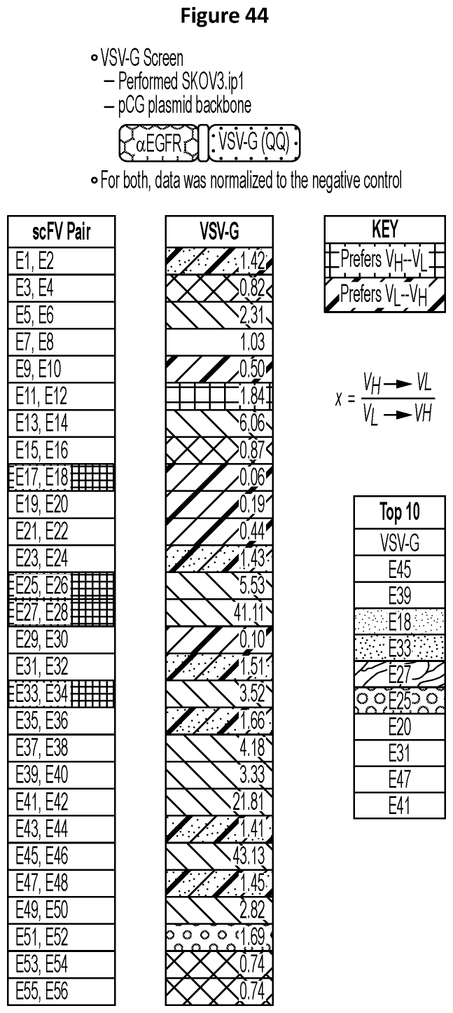

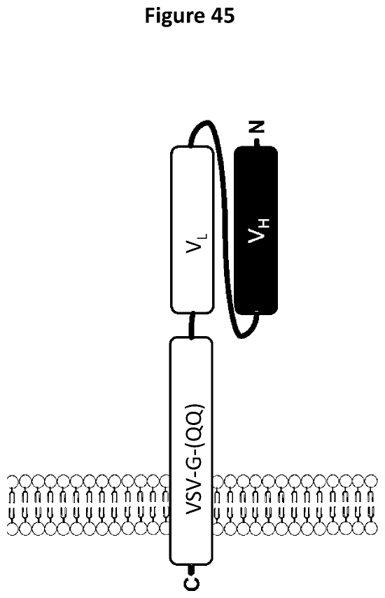

VSV-G DSP screen. Eleven αEGFR scFv-VSV-G plasmids outperformed the positive control (FIGS. 42-43), with four scFvs (including E18, E25, E27, E33) being the opposite orientation to those seen in a similar screening procedure involving an alternate virus protein not derived from a member of the Rhabdoviridae family. This led to further analysis of the difference in orientation by dividing the heavy-chain→light-chain orientation by light-chain→heavy-chain orientation to develop a heat map (FIG. 44). Using all the scFv combinations, the alternative virus background may prefer the light-chain- heavy-chain orientation, while VSV-G background may prefer the heavy-chain→light-chain orientation. Given the structure of the two proteins this would put the variable heavy-chain on the terminal end of the ectodomain of the glycoproteins (FIG. 45). This could aid in designing future screens and speeding up the process.

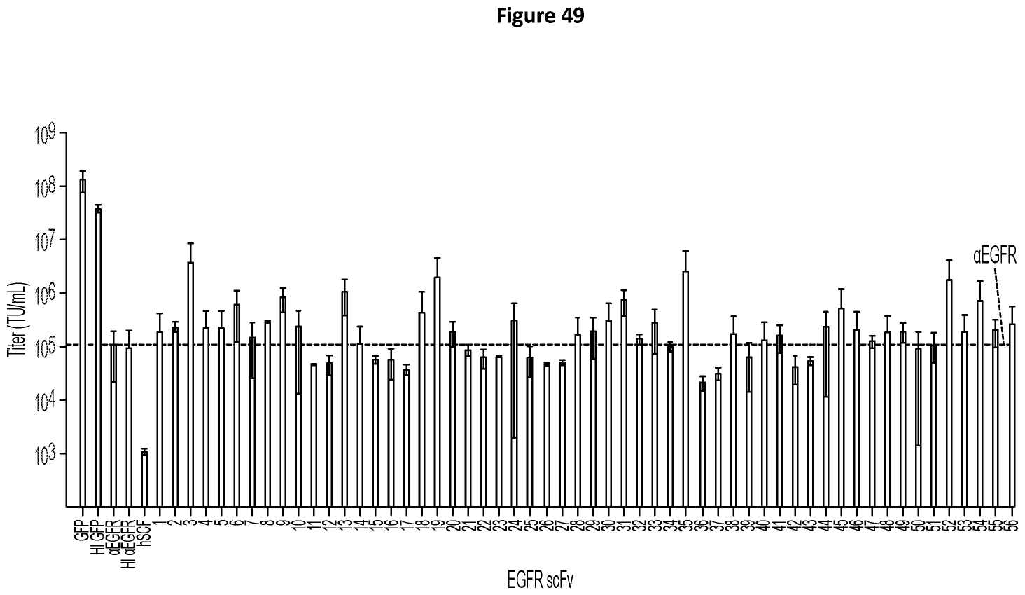

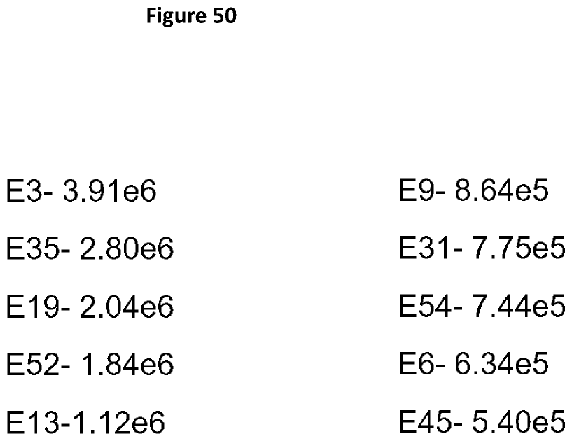

Lentivirus screen. One development of the present Example was the difference in results for the VSV-G DSP and VSV-G lentivirus screens with αEGFR scFv-VSV G plasmids. In the case of the lentivirus screen, several of the pCG-4MC11-αEGFR scfv-17aa-linker-VSV-G(QQ) plasmids produced lentivirus at the same titer or higher than the positive control containing other αEGFR scFv sequences (FIG. 49), with the top ten having titers 5- to 40-fold higher than control (FIG. 50). The produced pCG-4MC11-hSCF-17aa-linker-VSV G(QQ) lentivirus had a titer nearly 100-fold lower indicating that efficient transduction of the cells may require binding to the EGFR molecule on the surface of the cell. When comparing the top ten scFv sequences from both VSV G screens, there were two scFvs, E31 and E45, that were shared between the two screens (FIG. 50).

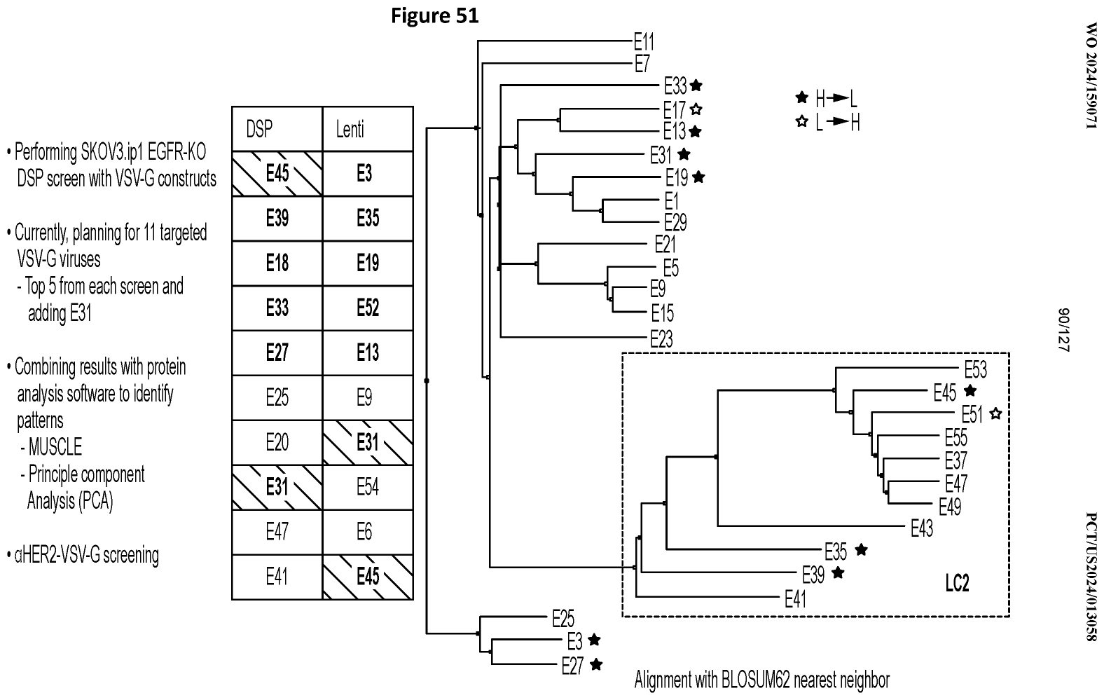

αEGFR scFv-VSV G screen analysis. Because both αEGFR scFv-VSV-G screens produced different results, the top five candidates from each screen were rescued, in addition to the E31 scFv sequence as the E31 scFv appeared in the top ten for both screening, thereby resulting in 11 viruses in total (in bold on Table in FIG. 51). A comparison of the top scFv candidates from both screens can narrow down to use of a single screen for future scFv targets; however, use of both screens may be the suitable option to quickly narrow down scFv sequences. Analysis of the 11 scFv sequences revealed two patterns. First, of the 11 sequences, only two of them were in the light-chain→heavy-chain orientation supporting the previous observation that VSV-G may prefer heavy-chain→light-chain orientation for scFv retargeting. Next, when scFv sequences were displayed on a phylogenetic tree (see, e.g., FIG. 51) the sequences can either cluster in a group of five or in three pairs. Based on sequence identity, the range is from 58.94% percent identity between E3 and E51 and 94.21% identity for E3 and E27, with a median identity of 72.69%. This suggest that while there are some differences between the candidates, there are some similarities amongst them on the protein sequence level.

### CONCLUSIONS

Rapid screening of scFv libraries can be important for selection of the proper scFv for retargeting VSV oncolytic viruses. The present Example demonstrates screening methods that identified top αEGFR scFv sequences for use in the VSV-G engineered platforms. With the VSV-G, the DSP screen efficiently identified αEGFR scFv sequences that were successfully rescued and amplified for in vivo testing. Preferred orientations for scFv sequences for VSV G, i.e., scFv linked to N-terminus of G, suggests that it is can be necessary to screen multiple scFv constructs against a given target to identify those that are capable of functional display on an underlying G protein. This may be a consequence of incompatibility between certain scFv constructs and the underlying G protein, leading to interference with protein folding and/or transport to the cell surface.

While the VSV-G lentivirus screen produced different results compared to the VSV-G DSP screen, a number of commonalities including scFv orientation, two αEGFR scFv sequences, and highly conserved sequences by pair-wise analysis, especially E3 and E27, were present between the two assays. The difference seen between the two assays emphasizes the importance of having multiple assays to develop a deeper understanding of the biology scFv retargeting of VSV to aid in selecting scFv for future oncolytic treatments.

### Methods

Generation of αEGFR scFv-VSV-G library. To generate the VSV-G library, scFv sequences were subcloned into pCG-4MC11-αEGFR-17aa linker-VSV G(QQ) using NotI and SfiI digestion sites. The pCG-4MC11-αEGFR-17aa linker-VSV G(GG) contained K47Q and R354Q blinding mutations to prevent binding of the VSV-G to its native target receptor LDL receptor and another αEGFR scFv. Insertion of each αEGFR scFv sequence using the NotI and SfiI sites allowed for rapid generation of the library and removed the 6×His-tag (SEQ ID NO: 235) from the scFv sequence added in the previous library. This removal was necessary as insertion of a 6×His-tag (SEQ ID NO: 235) can be prohibitive for VSV-G binding.

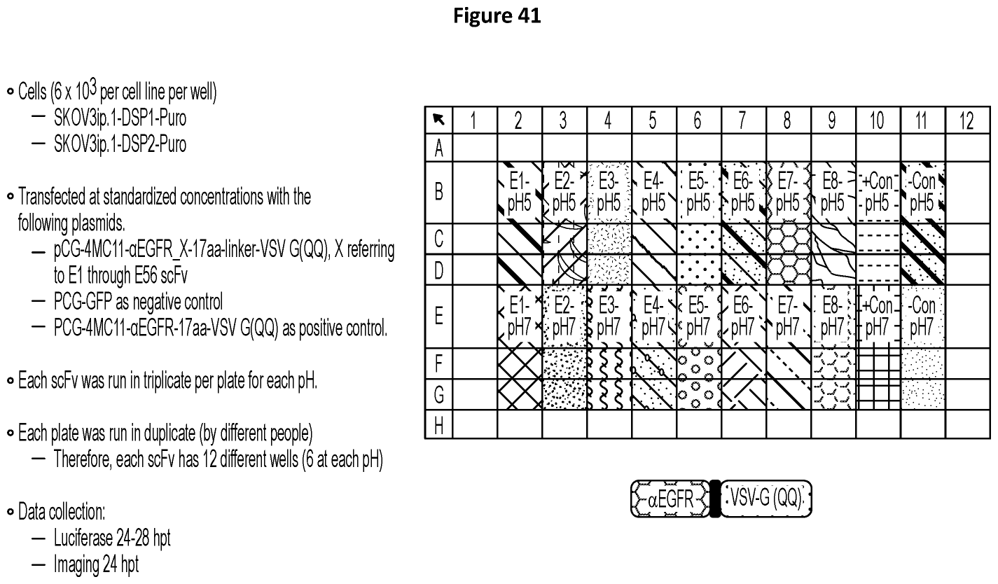

DSP screen of pCG-4MC11-αEGFR scfv-17aa-linker-VSV G(QQ) library. 6,000 cells of both SKOV3ip.1-DSP1 and -DSP2 cells were plated into each well of a 96-well plate, with the outer rim of well containing PBS to prevent evaporation. The next day, jetPrime reagent was used to co-transfect the wells with 200 ng of either a pCG-4MC11-αEGFR scfv-17aa-linker-VSV G(QQ) plasmid from the library or a control plasmid, with pCG-GFP being the negative control and pCG-4MC11-αEGFR-17aa-linker-VSV G(QQ) containing a published scFv as the positive control. Each plasmid was transfected into a total of six wells to be used a triplicate for a pH 5.0 and pH 7.0 PBS shock to elicit fusion (see, e.g., FIG. 41, for diagram). VSV-G can require a lower pH to promote fusion and pH 7.0 served as a control. At 22 h, the media was replaced with PBS at either pH 5.0 or pH 7.0 for 2 min to promote fusion, after which PBS was replaced with media containing 7.5 μM EnduRen (Promega) and incubated for 2 h. At 24 h, luciferase readout was measured with a Tecan set for an integration of 2 sec and settle time of 200 ms. Each well was imaged with a Celigo Imaging Cytometer using both phase and GFP settings.

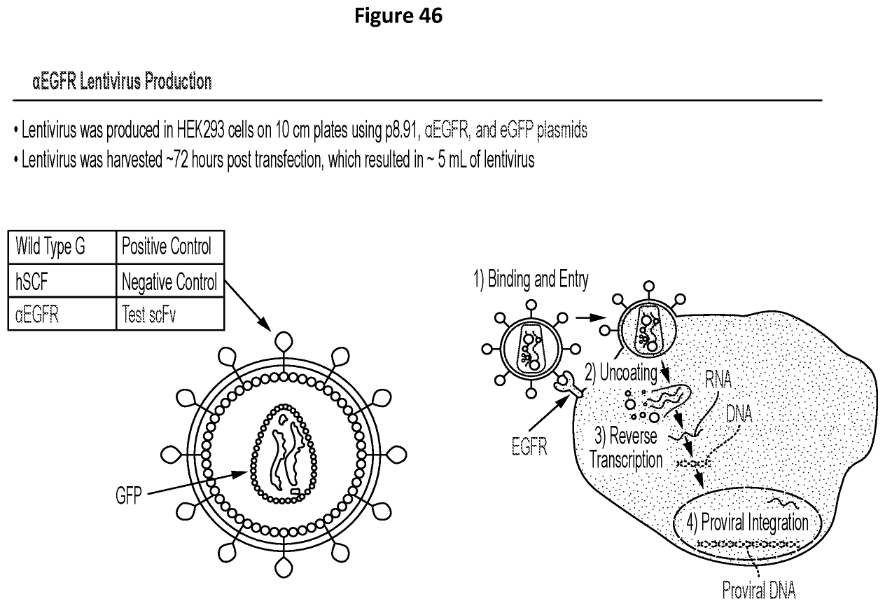

Lentivirus screen of pCG-4MC11-αEGFR scfv-17aa-linker-VSV G(QQ) library. 3.5×10⁶ HEK-293T cells were plated onto poly-D-lysine coated plates. The next day, the plates were transfected with 2 μg of a GFP transfer cassette (DNA1023; Imanis Life Sciences), 2 μg p8.91, and 1 g of either a pCG-4MC11-αEGFR scfv-17aa-linker-VSV G(QQ) plasmid from the library or a control plasmid, with pCG-VSV-G an untargeted control and pCG-4MC11-αEGFR-17aa-linker-VSV G(QQ) containing a published scFv as the positive. For a negative control, a previously produced pCG-4MC11-hSCF-17aa-linker-VSV G(QQ) which contains human stem cell factor (hSCF) and may not bind to the target titration cell line was used which was functionally confirmed on other cells. After 18 h, media was replaced with Opti-MEM (Gibco). At 3 days post transfection, the media was collected, debris was pelleted out by centrifugation at 500×g for 5 min and stored at −80° C. until titrations (FIG. 46).

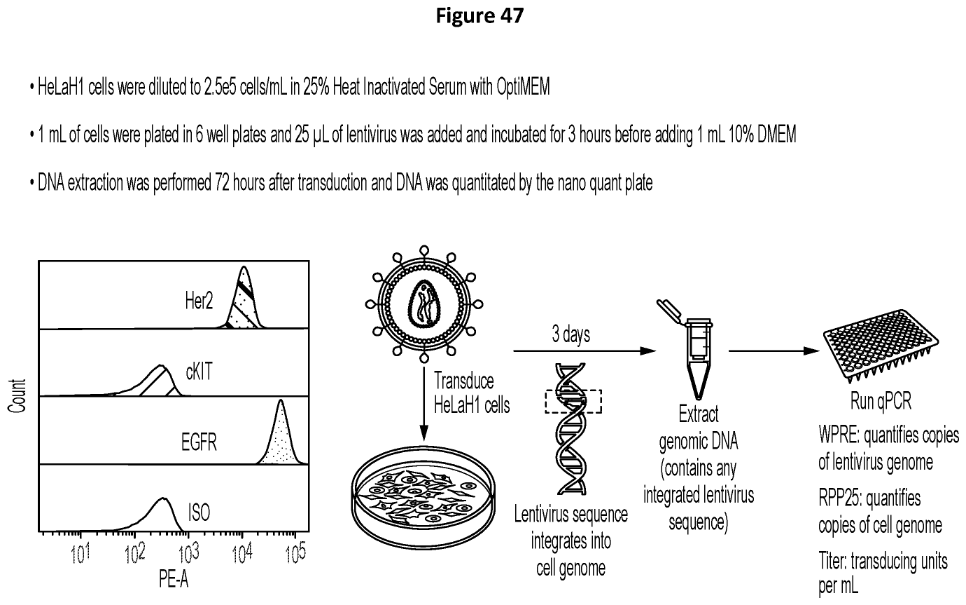

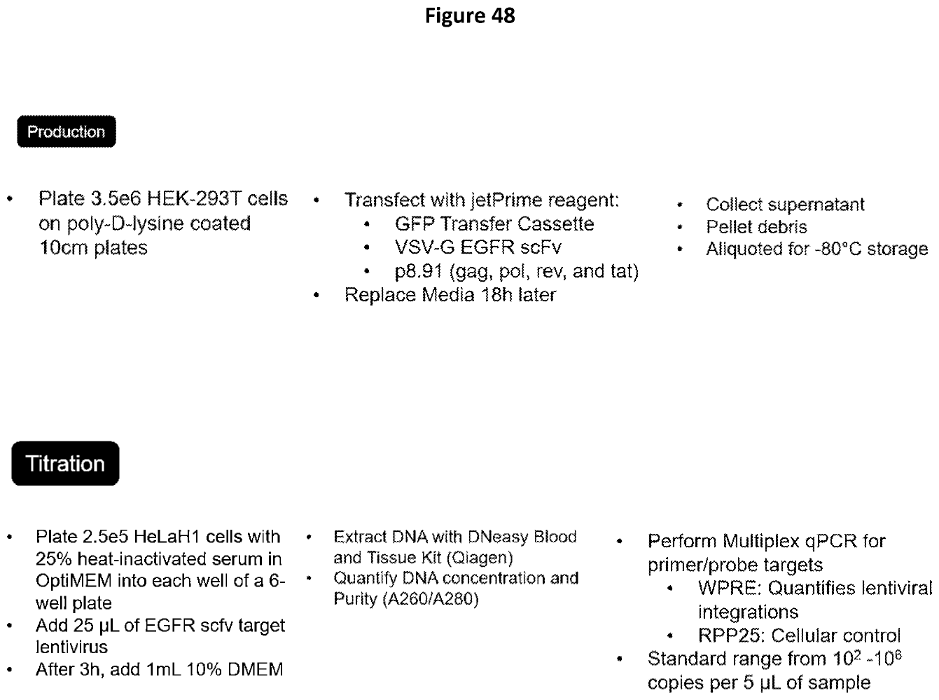

Lentivirus titration by qPCR was used as the readout for the lentivirus screen. 2.5×10⁵ HeLaH1 cells were plated with 25% heat-inactivated serum in Opti-MEM into 6-well plates (FIG. 47). Immediately after plating, 25 μL of harvest lentivirus was added to the well. Included control transductions without the 25% serum as additional controls. After 3 h, 1 mL of DMEM containing 10% fetal bovine serum (FBS) was added to the well. The 25% serum was used to inhibit VSV infection through LDLR by adding LDL, which is found in serum, to the culture. After 3 days, DNA was isolated from the cells using DNeasy Blood and Tissue Kit (Qiagen) and measured for concentration and purity (A260/A280) using the Tecan. The qPCR was carried out using LightCycler 480 Probes Master (Roche) kit, 3 ng/μL isolate DNA, WPRE primer/probe mix (quantifies lentivirus), and RPP25 primer/probe mix (quantifies cellular control). Plates include standards to quantify copy numbers from 10²-10⁶ copies per 5 μL. All samples, standards, and controls were titered in triplicate (FIGS. 46-48).

Analysis. For both DSP assays, the samples were normalized within the plates by dividing the samples by the negative control to account for plate-to-plate variation. These values were then graphed using GraphPad software to visualize the values and determine top candidates for VSV virus rescue (FIG. 43). Additionally, to understand the importance of scFv orientation, the heavy-chain→light-chain orientation by light-chain→heavy-chain orientation were divided to develop a heat map (FIG. 44). With values ≥4 having a stronger preference for the heavy-chain→light-chain orientation, while values ≤0.25 having a stronger preference for the light-chain→heavy-chain orientation.

For the lentivirus screen, titers were calculated from the three replicates and displayed on a graph using GraphPad with the positive control indicated with a dotted line (FIG. 49). To compare the DSP and lentivirus screens for the αEGFR-VSV G constructs, the scFv sequences were compared using Multiple Sequence Comparison by Log-Expectation (MUSCLE; ebi.ac.uk/Tools/msa/muscle/) and graphically represented in a phylogenetic tree aligned using Jalvuew software and BLOSUM62 nearest neighbor algorithm (FIG. 51). The scFv sequences aligned are only in the heavy-chain 4 light-chain orientation as comparison of both orientations initially results in the sequences separating by orientation and makes the figure more difficult to display. Stars are used to indicate the position of the eleven viruses proposed for VSV rescue.

### Example 38. Design and Characterization of Linkers

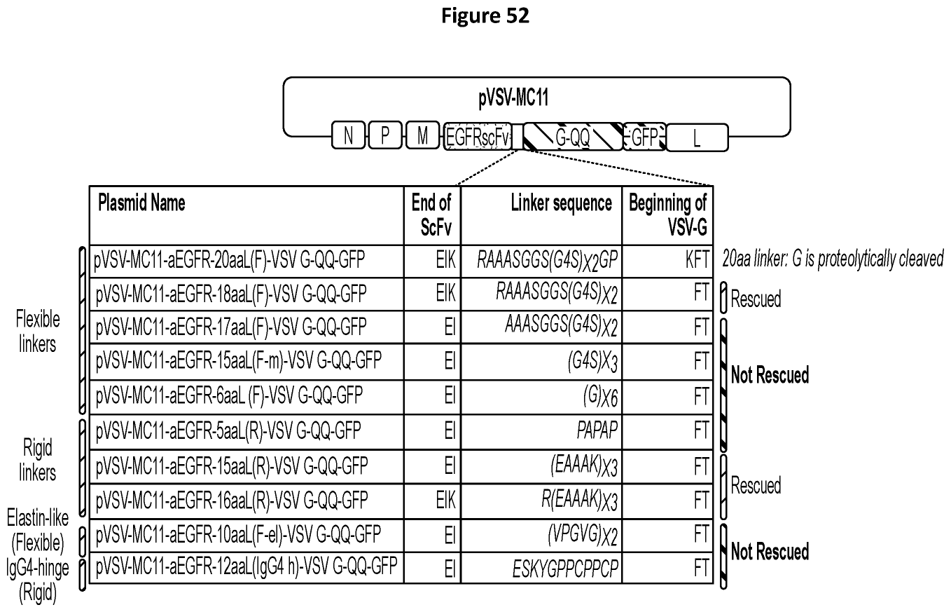

Constructs were designed with various linker sequences (FIG. 52). Retargeted VSV containing a 19 as linker between the EGFR scFv and VSV-G for proteolytic cleavage was also included in the analysis. Specifically, to test the effect of alternate linkers on G-cleavage, different types of linker sequences with variable lengths were cloned between the EGFR scFv and VSV-G. The amino acid sequence EIIKR was the terminal amino acid sequence of the scFv. The amino acid sequence KFT was the starting amino acid sequence of VSV-G. Linker sequence included flexible, rigid, medium flexible, flexible elastin-like, and IgG4 hinge designs. The VSV-G contained K47Q and R354Q blinding mutations to LDLR. A description of cleavable and uncleavable linkers of the present Example is provided in Table 5. The linker sequences are shown in underlined text. KFT or FT is the starting amino acid (aa) sequence of the VSV-G protein. EIK and LGAK (SEQ ID NO: 59) are the terminal amino acid sequences of EGFR scFv and Her2 scFv, respectively.

TABLE 5
Summary of cleavable and uncleavable linkers

| | | | Linker sequence + | | |
|---|---|---|---|---|---|
| | | End of | starting sequence | Virus | |
| Virus ID | Virus Name | scFv | of VSV-G (aa) | Rescuc | G Cleaving |
| VSV- | VSV-MC11-G | EIK | RAAARGSPK(G₄S)₃KFT | YES | Cleaved |
| 1114-0 | αEGFR_HvL.v | | (SEQ ID NO: 164) | | |
| | (K47Q R354Q)-GFP | | | | |
| VSV- | VSV-G | LGAK | (G₄S)₃GPKFT | YES | Cleaved |
| 1108-0 | αHER2scFv_HvL.v | (SEQ ID | (SEQ ID NO: 23) | | |
| | (K47Q R354Q)-GFP | NO: 59) | | | |
| VSV- | VSV-MC11- | EIK | RAAA(G4S)₃KFT | YES | Cleaved |
| 1306-1 | αEGFR-Not1- | | (SEQ ID NO: 165) | | |
| | Linkcr-VSV G | | | | |
| | K47QR354Q-GFP | | | | |
| VSV- | VSV-MC11- | EIK | RAAASGGS(G₄S)₂GPKFT | YES | Cleaved |
| 1310-3 | αEGFR-20aa-linker- | | (SEQ ID NO: 166) | | |
| | VSV G K47Q | | | | |
| | R354Q-GFP | | | | |
| VSV- | VSV-MC11- | LG | AAASGGS(G₄S)₂GPKFT | YES | Cleaved |
| 1308-1 | αHER2-HvLv-dAK- | | (SEQ ID NO: 25) | | |
| | 19aa-linker-VSV G | | | | |
| | K47Q R354Q-GFP | | | | |
| VSV1- | VSV-MC11- | EIK | RAAASGGS(G₄S)₂FT | YES | Cleaved |
| 382-0 | αEGFR-KR- | | (SEQ ID NO: 167) | | |
| | 18aaL(F)-VSV G- | | | | |
| | QQ-GFP | | | | |
| VSV1- | VSV-MC11- | EI | AAASGGS(G₄S)₂FT | NO | Uncleaved |
| 383-0 | αEGFR-17aaL(F)- | | (SEQ ID NO: 26) | | |
| | VSV G-QQ-GFP | | | | |
| VSV1- | VSV-MC11- | EI | (G₄S)₃FT | NO | Uncleaved |
| 384-0 | αEGFR-15aaL(F-m)- | | (SEQ ID NO: 27) | | |
| | VSV G-QQ-GFP | | | | |
| VSV1- | VSV-MC11- | EI | (G)₆FT | NO | Uncleaved |
| 386-0 | αEGFR-6aaL (F)- | | (SEQ ID NO: 28) | | |
| | VSV G-QQ-GFP | | | | |
| VSV1- | VSV-MC11- | EI | PAPAPFT | NO | Uncleaved |
| 387-0 | αEGFR-5aaL(R)- | | (SEQ ID NO: 29) | | |
| | VSV G-QQ-GFP | | | | |
| VSV1- | VSV-MC11- | EI | (EAAAK)₃FT | YES | Cleaved |
| 388-0 | αEGFR-15aaL(R)- | | (SEQ ID NO: 30) | | |
| | VSV G-QQ-GFP | | | | |
| VSV1- | VSV-MC11- | EIK | R(EAAAK)₃FT | YES | Cleaved |
| 389-0 | αEGFR-KR- | | (SEQ ID NO: 168) | | |
| | 16aaL (R)-VSV G- | | | | |
| | QQ-GFP | | | | |
| VSV1- | VSV-MC11- | EI | (VPGVG)₂FT | NO | Uncleaved |
| 390-0 | αEGFR-10aaL(F- | | (SEQ ID NO: 31) | | |
| | el)-VSV G-QQ-GFP | | | | |
| VSV1- | VSV-MC11- | EI | ESKYGPPCPPCPFT | NO | Uncleaved |
| 391-0 | αEGFR-12aaL(IgG4 | | (SEQ ID NO: 32) | | |
| | h)-VSV G-QQ-GFP | | | | |

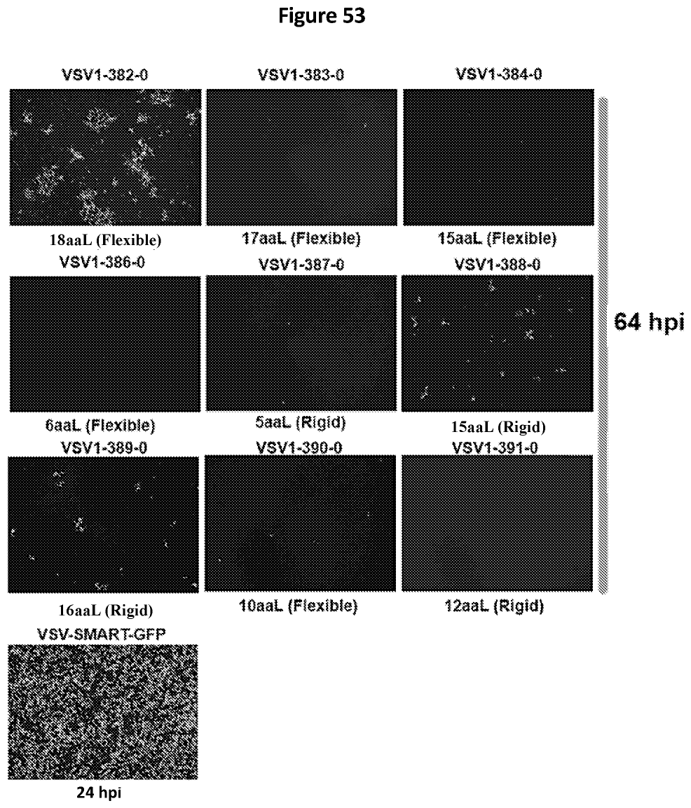

To test rescue of EGFR targeted VSV with alternate linker sequences, pVSV-MC11-EGFRscFv-VSV-G-GFP plasmids with alternate linker sequences were rescued in SKOV3.ip1 cells using a vaccinia-based rescue system. At 3 days post-transfection, virus supernatant was collected and filtered which was then referred to as p0 supernatant (sup). The p0 supernatant (1 ml) was then transferred to a monolayer Vero-EGFR cells and the fluorescence microscopy images were acquired at 64 hpi (FIG. 53). Data revealed only three retargeted VSVs containing 17 aa flexible linker and 15 aa rigid linker sequences showed GFP-positive cells, thus indicating rescuing these VSVs was successful.

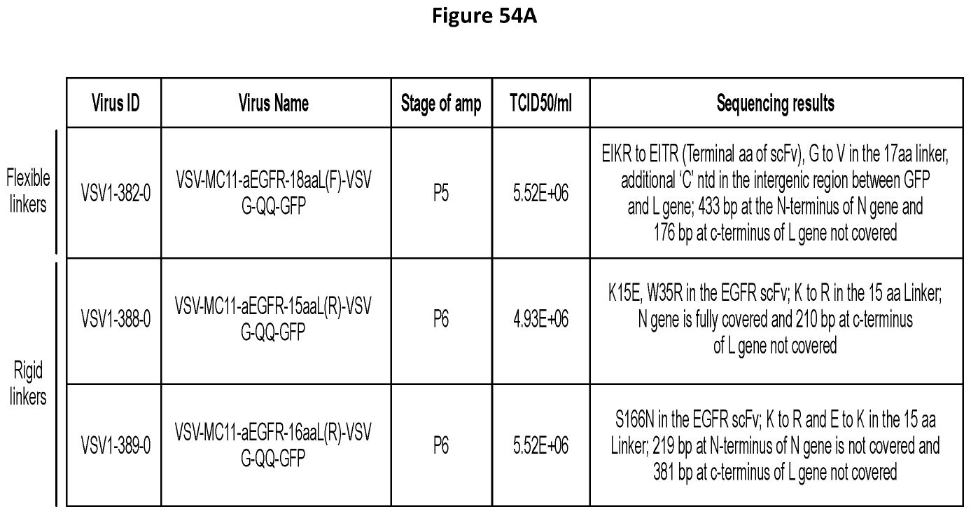

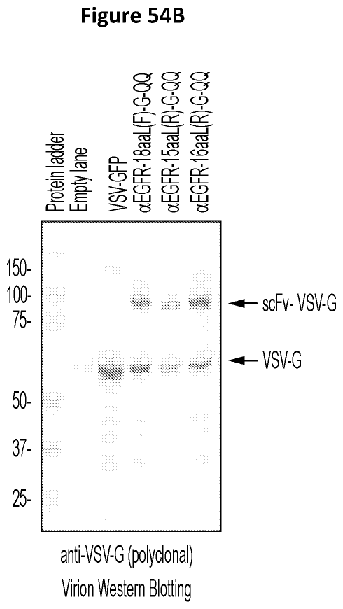

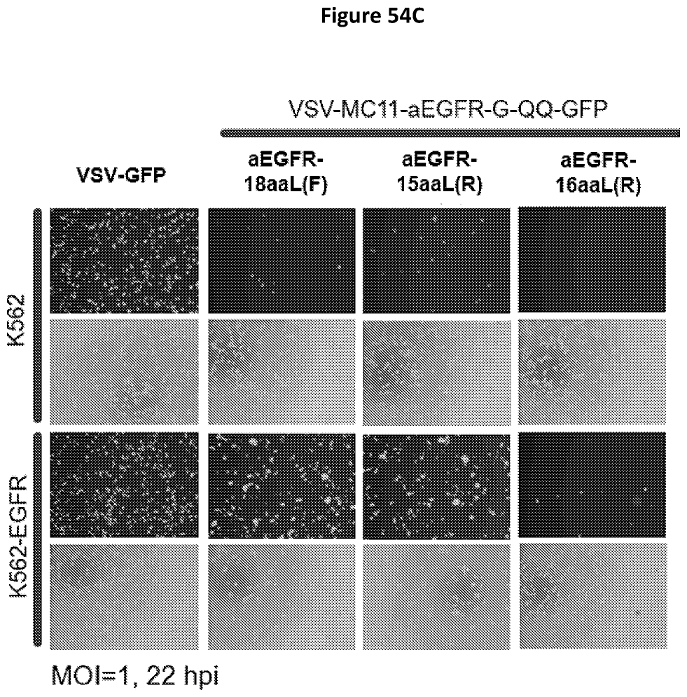

Amplification stage, virus titers and sequencing results for EGFR-targeted VSVs are shown in FIG. 54A. For western blotting of virions, VSV-GFP or retargeted VSV were pelleted and subjected to western blotting with anti-VSV-G polyclonal antibody. Bands showing EGFR scFv intact with the G and the proteolytically cleaved G were detected at predetermined molecular weights (FIG. 54B). To test the specificity of the EGFR targeted VSVs, K562 parental or K562-EGFR were infected with EGFR-targeted VSV along with VSV-GFP as a control at an MOI of 1.0. Fluorescence micrographs were acquired at 22 hpi (FIG. 54C). Data showed that αEGFR-15aaL(R) virus displayed reduced infection compared to other retargeted viruses. VSV-GFP showed comparable infection on K562 parental and K562-EGFR cells while 18aaL (F) or 16aaL (R) linker VSVs showed specific infection on K562-EGFR cells with little background infection on K562 parental cells. These data indicated that EGFR-targeted VSVs with alternate linkers which were proteolytically cleaved were able to rescue the VSVs and demonstrated retargeting specificity on the EGFR positive cells.

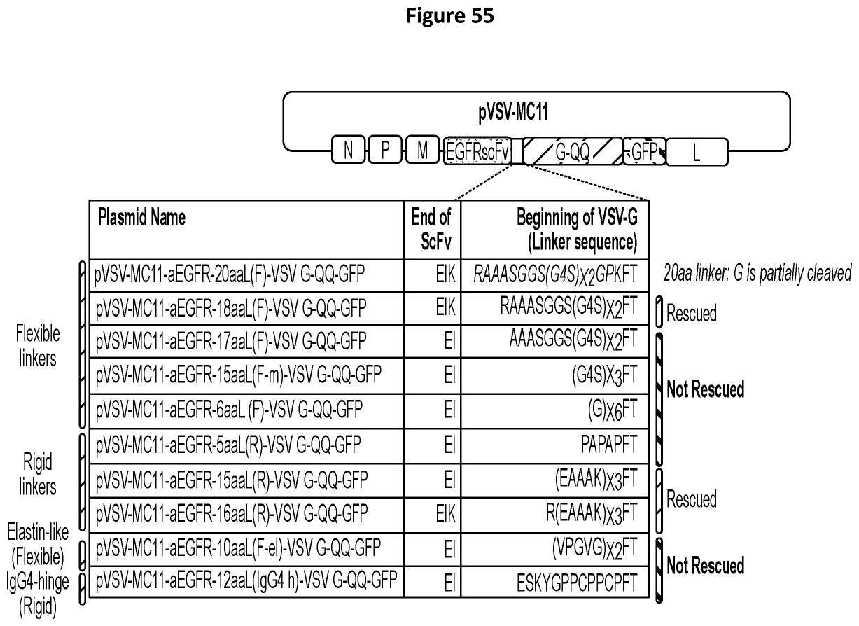

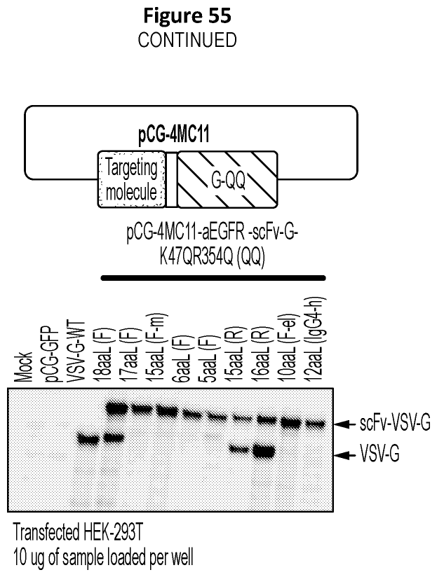

To test whether 18aaL(F), 15aaL(R), and 16aaL(R) linkers exhibited proteolytic cleavage, nine different linkers attached to EGFR scFv were selected based upon various properties including, for example, length, flexibility/stiffness, hydrophobicity/hydrophilicity, and potential susceptibility to partial proteolytic cleavage to determine which linker will provide the most specific targeting to EGFR (FIG. 55, left panel). The linker sequences were initially produced in the VSV backbone and then subcloned into the pCG vector backbone for use in lentivirus production. Constructs were transfected into HHEK293T cells. 48 hours post transfection, cell lysates were collected and run on an SDS-PAGE gel to analyze protein expression. The western blot shown on the right in FIG. 55 suggests that three constructs: 18aaL(F), 15aaL(R), and 16aaL(R), exhibited proteolytic cleavage, while the remaining constructs had an intact EGFR scFv-linker-VSV-GQQ molecule and were not cleaved. Constructs that cleaved contained a K or R in either the linker sequence or at the C-terminus of EGFR scFv, suggesting that K and/or R can be targeted for cleavage.

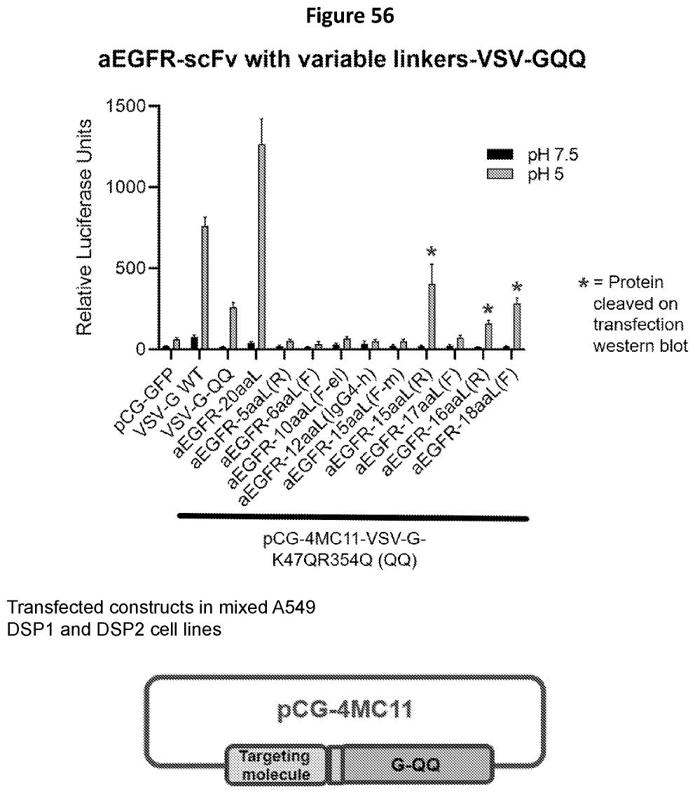

To measure fusion activity of the 9 constructs that contained EGFR scFv with variable linkers, a mixed population of A549-EGFR expressing DSP1-7 and DSP8-11 cells were transfected. Half contained a dual-split protein (DSP) reporter gene with a split GFP and half contained Renilla luciferase. A plasmid expressing GFP served as a negative control for fusion and non-targeted VSV-G WT, pseudotyped VSV-G-K47QR354Q (QQ), and αEGFR-20aaL(F)-VSV-GQQ served as positive controls for fusion. One day after transfection, the media was removed and replaced with either neutral pH 7.0 or pH 5.0 PBS for 2 minutes. The PBS was then replaced with fresh media containing the EnduREN luciferase substrate and the luminescence was recorded 2 hours later. The results showed that the three constructs that exhibited proteolytic cleavage (18aaL(F), 15aaL(R), and 16aaL(R)) show an increased RLU value, indicating fusion can occur with these three linker constructs (FIG. 56).

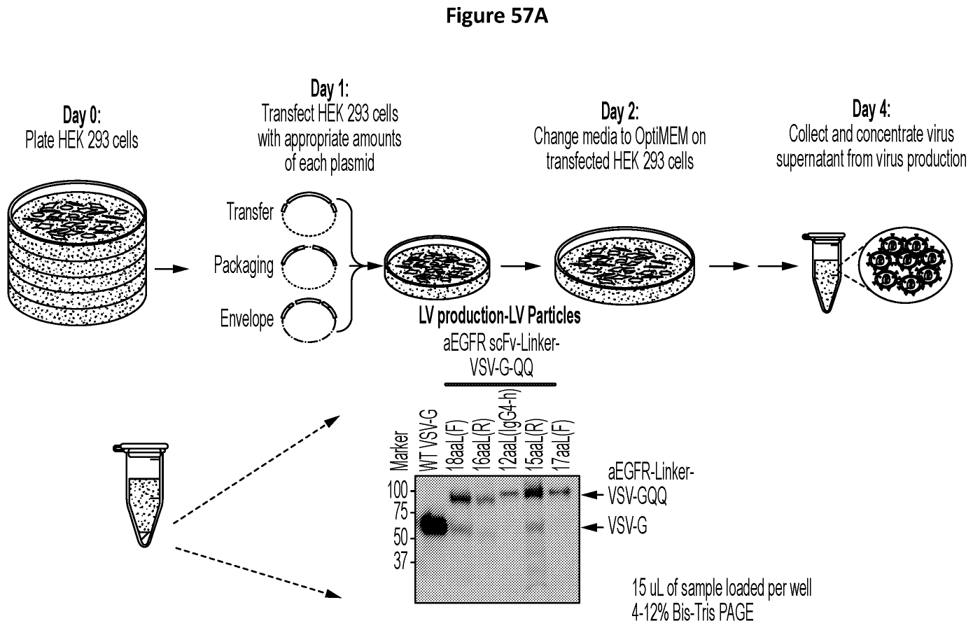

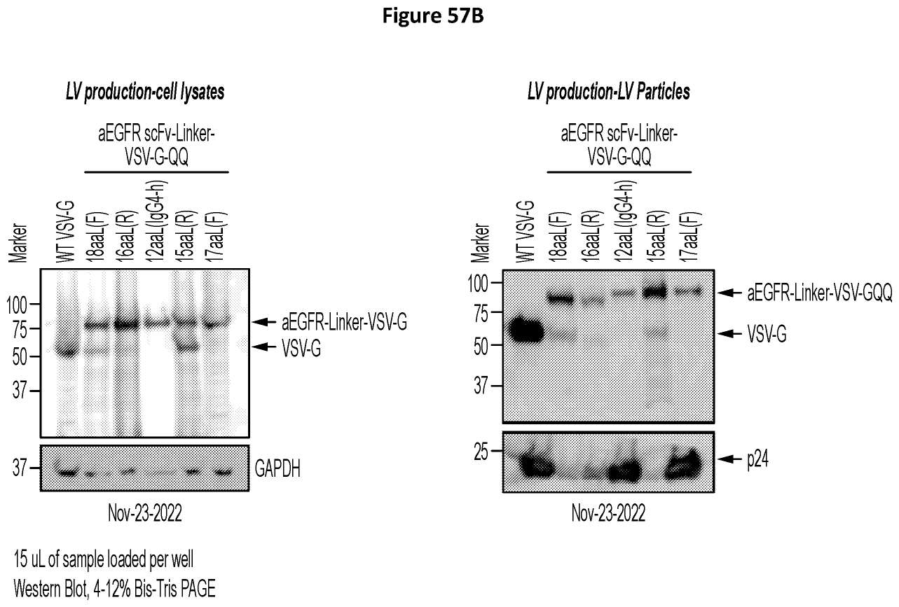

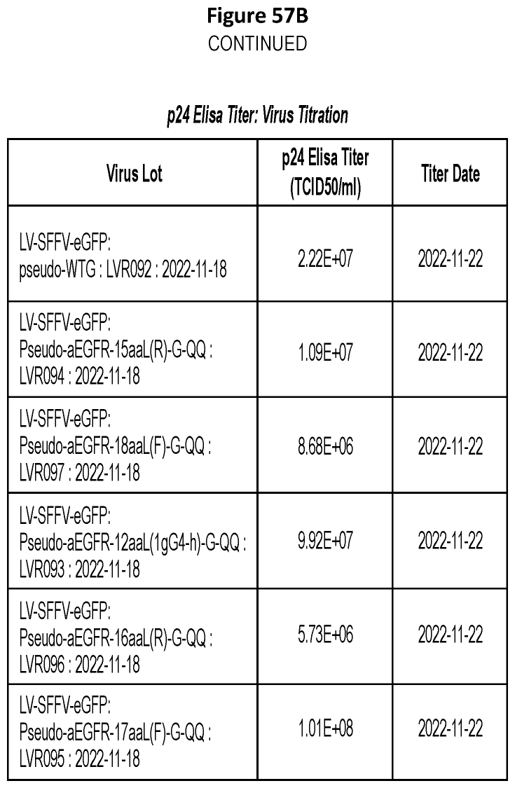

To test whether 18aaL(F), 15aaL(R), and 16aaL(R), linkers exhibited proteolytic cleavage in lentivirus production cell lysates and the lentivirus particle itself, lentivirus production plasmids were transfected into HEK 293T cells to produce lentiviruses (FIG. 57A). Cell lysates from cells used to produce the lentiviruses were harvested, lysed, reduced at 95° C. for 5 minutes and analyzed using western blotting to detect EGFR scFv-linker-VSV-GQQ (full length) or the cleaved VSV-GQQ (FIG. 57B, left panel). The supernatant that contained the virus particles was collected and spun down in a microcentrifuge for 3 hours at 14,000 RPM at 4° C. The supernatant was aspirated, and the cell pellet was lysed, reduced at 95° C. for 5 minutes, and then run on an SDS-PAGE gel to detect EGFR scFv-linker-VSV-GQQ (full length) or the cleaved VSV-GQQ (FIG. 57B, middle panel). The three constructs that cleaved previously when transfected alone on HEK293 Ts (18aaL(F), 15aaL(R), and 16aaL(R)) also retained partial cleavage in the cell lysates and on the virion particles. The titer of each virus was then determined by p24 ELISA (FIG. 57B, right panel).

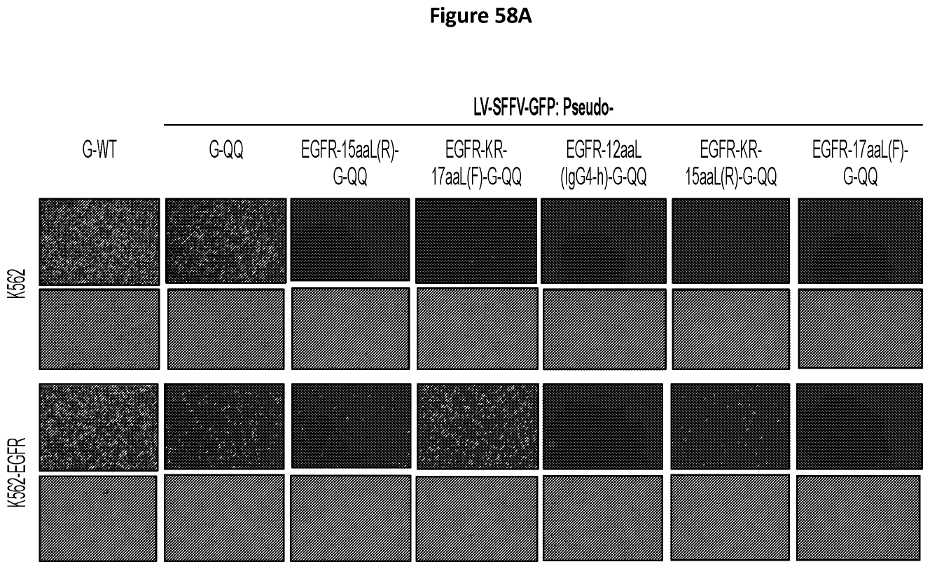

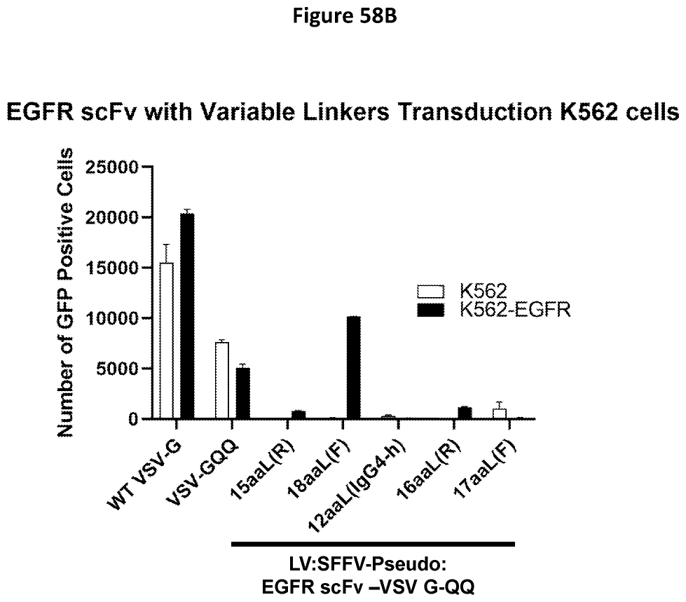

To determine whether 18aaL(F), 15aaL(R), and 16aaL(R) linkers that exhibited proteolytic cleavage also demonstrated increased specificity for targeting K562-EGFR expressing cells, K562 parental and K562-EGFR expressing cells were first transduced at an MOI of 5 with each lentivirus. GFP fluorescence microscopy and bright field (BF) images were captured at 72 hours post transduction to determine specificity of each retargeted lentivirus with a different linker (FIG. 58A). The number of GFP positive cells was quantified in K562 and K562-EGFR cells transduced with each lentivirus FIG. 58B. The results show that the 18aaL(F), 15aaL(R), and 16aaL(R) constructs that exhibited proteolytic cleavage, also demonstrated increased specificity for EGFR expressing K562 cells.

### Example 39. Evaluation of Virus Specificity of Targeting Constructs in the Presence or Absence of Serum

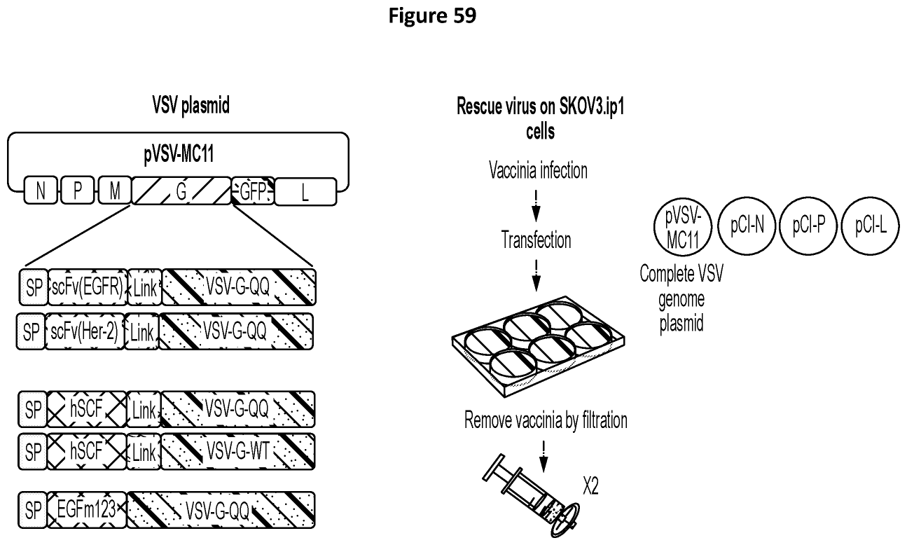

The present Example evaluated virus specificity of targeting constructs in the presence and absence of serum. The targeting constructs used in this Example are shown in FIG. 59. Displayed ligands included human EGF, human stem cell factor (hSCF) and scFvs against EGFR or HER2. The hSCF ligand was displayed on a blinded (VSV-G-QQ) G protein that has poor interaction with LDLR, or an unblinded (VSV-G-WT) G protein. For generation of data displayed in FIG. 60, K562 cells, parental or stably expressing HER2, cKit (SCF receptor) or EGFR were infected in the absence (FIG. 60, left panel) or presence (FIG. 60, right panel) of 25% heat inactivated human serum, with recombinant VSVs incorporating the G proteins shown in FIG. 59. Infected cell monolayers were imaged under blue light 42 hpi (24 hpi for VSV-GFP with unmodified G protein). Ligand-displaying viruses specifically infected only those target cells that bore the cognate receptor for their displayed ligand (FIG. 60, left panel). This was apparent even for the SCF-displaying virus wherein the G protein had not been mutated to ablate LDLR tropism, indicating that the displayed SCF domain could sterically interfere with the G protein-LDLR interaction. In the presence of 25% heat inactivated human serum, entry of the virus bearing a wild type G protein was blocked in all K562 clones, whereas the entry of targeted viruses via alternate (i.e., non-LDLR) receptors was not blocked, and may even be enhanced in certain cases (FIG. 60, right panel).

### Example 40. Evaluation of Mixed Trimers

Various linkers were previously attached to EGFR scFv and a blinded VSV G mutated at K47Q and R354Q (QQ). The 18aaL(F), 15aaL(R), and 16aaL(R) linkers all showed targetability and specificity in the K562-EGFR expressing cells and exhibited protein cleavage. In contrast, the 17aa(F) and 12aaL(IgG4-h) demonstrated neither proteolytic cleavage nor specific targeting. It was hypothesized that cleavage can be necessary for proper trimerization and entry into the cell. Therefore, whether other retargeted lentiviruses with the 12aaL(IgG4-h) or 17aaL(F) linker could not target and form proper trimers due to steric hinderance, potentially from an inability to cleave, was tested. To address this and determine if cleavage could be bypassed, lentiviruses that contained a mixed ratio of EGFR scFv-17aaL(F)-VSV G-QQ and VSV G-QQ were produced. The 17aaL(F) virus was chosen because it is close in amino acid sequence to the 18aaL(F) linker that did cleave and target specifically. K562 and K562-EGFR expressing lymphoblast cells were then transduced to determine targeting specificity for each virus. Traditional 2^{nd} generation lentivirus production includes one envelope glycoprotein plasmid and not two. However, two glycoproteins could be combined because the same total amount of the envelope DNA was maintained in production.

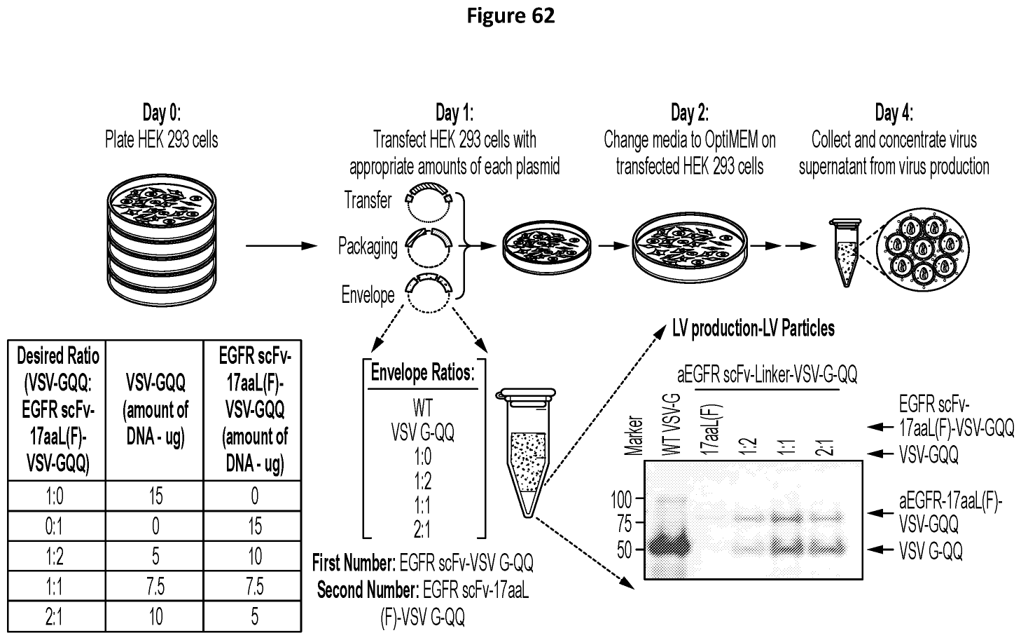

Lentiviruses were produced as follows (see, e.g., FIG. 62): HEK 293 kidney epithelial cells were plated at approximately 75% confluency onto five 15-cm plates per virus and cultured in 10% Fetal Bovine Serum (FBS) Dulbecco's Modified Eagle Media (DMEM). The following day, each virus' group of plates were transfected with the transfer, packaging, and envelope plasmids. The amount of DNA used was the same for all three plasmids. However, for the envelope plasmid of each virus, the appropriate ratio of EGFR scFv-17aaL(F)-VSV G-QQ and VSV G-QQ plasmids was combined. The following are the ratio and amounts of DNA used for each ratio: 1:0-15 μg VSV G-QQ and 0 μg of EGFR scFv-17aaL(F)-VSV G-QQ; 0:1-0 ug VSV G-QQ and 15 μg of EGFR scFv-17aaL(F)-VSV G-QQ; 1:2-5 ug VSV G-QQ and 10 μg of EGFR scFv-17aaL(F)-VSV G-QQ; 1:1-7.5 ug VSV G-QQ and 7.5 ug of EGFR scFv-17aaL(F)-VSV G-QQ; and 2:1-10 ug VSV G-QQ and 5 μg of EGFR scFv-17aaL(F)-VSV G-QQ. These values are also described within the table depicted in FIG. 62. Once plasmids were appropriately combined, the transfection mix was added drop-wise onto each plate. The next day, the media was changed and OptiMEM with no FBS was added. Three days later, the virus supernatant was collected and transferred to 50 mL conical tubes and centrifuged at 500×g for 5 mins at 4° C. The supernatant was then filtered through a 0.4 μM filter and concentrated with Lenti-X concentrator (1 part Lenti-X concentrator to 3 parts aliquoted supernatant). Samples were then incubated on ice for 6 hours at 4° C. Conical tubes were then centrifuged at 1250×g for 45 minutes at 4° C. Supernatant was carefully removed without disturbing the pellet. Finally, the pellet was resuspended in OptiMEM at approximately 10% of the starting volume. Pre-labeled tubes were aliquoted and stored at −80° C.

A virion pellet western blot was conducted to test for incorporation of the retargeted molecule into the virion particle (see western blot in FIG. 62). Virus supernatants of 1 mL were centrifuged in a microcentrifuge tube for 3 hours at 14,000 RPM and 4° C. After centrifugation, the supernatant was carefully removed with a p1000 pipette ensuring that 5 μLs was left in the tube to avoid disturbing the pellet. 20 Ls μ of lysis buffer (4×NuPage LDS with 2-mercapto-ethanol reducing agent) were added to each sample and then samples were heated at 95° C. for 5 minutes, removed and spun down to collect lysate at the bottom of the tube. 15 uLs of each boiled sample was loaded into each well of a NuPAGE Bolt 4-12% Bis-Tris 1 mm thick gel. The gel was run at a constant 150V for 60-70 minutes until the dye ran into the buffer. Proteins were transferred to a polyvinylidene difluoride (PVDF) membrane, blocked for 1 hour in 5% milk and 1×TBST. Anti-VSV G [8G5F11] antibody was used to probe for VSV G protein in the virion pellet overnight at 4° C. The next day, the blot was washed with 1×TBST 3 times, rabbit secondary antibody was added for 1 hour, then the blot was washed again with 1×TBST for 3 times and imaged on the ChemiDoc imaging system. The retargeted chimeric monomer showed up at the higher molecular weight and the VSV G-QQ showed up at the lower molecular weight and both increased in protein amount proportionally.

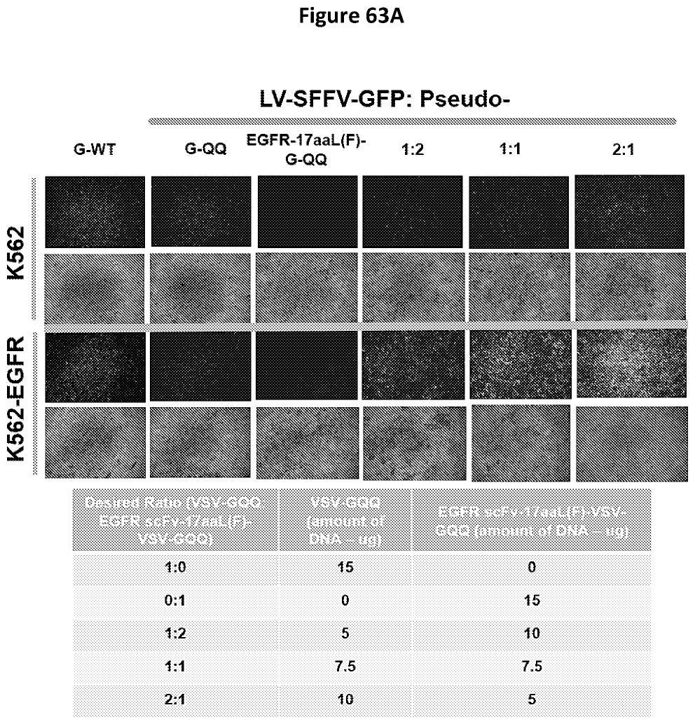

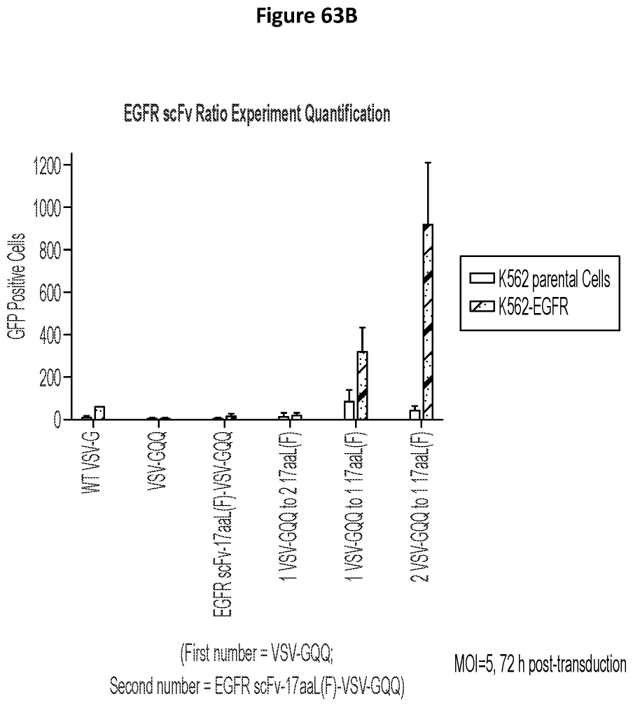

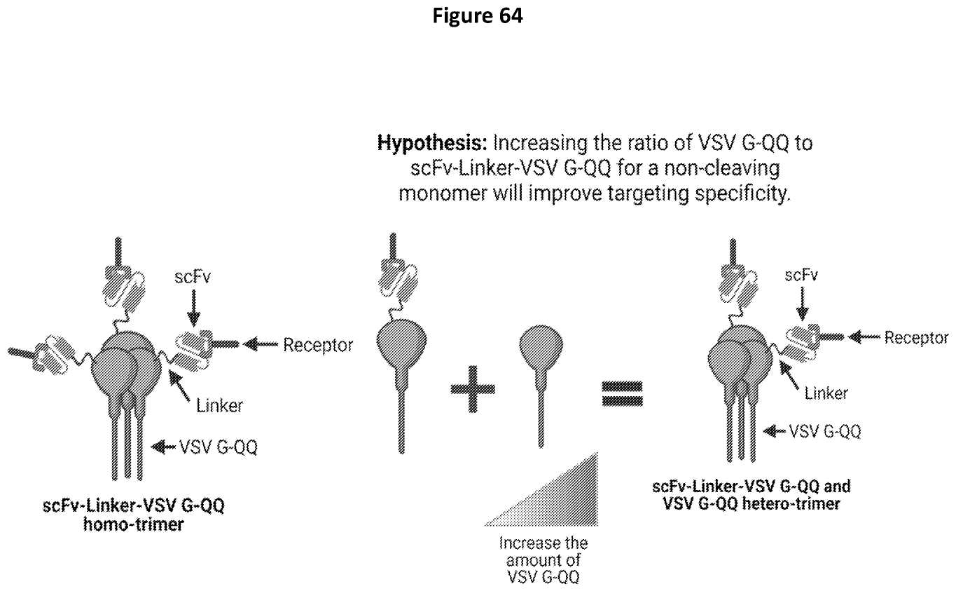

Each mixed ratio lentivirus was then transduced at an MOI=5 onto K562 and K562-EGFR expressing cells in triplicate and then three days later imaged and quantified for GFP positive cell number on the Celigo (FIG. 63A). The GFP positive cell quantifications were graphed using GraphPad Prism 9 and are shown in the bar graph of FIG. 63B. As the ratio of VSV G-QQ increased to EGFR scFv-17aaL(F)-VSV G-QQ, the specificity and targetability of the EGFR retargeted virus increased. This finding suggested that to have a functional trimer and reduce steric hinderance for linkers that cannot cleave, a smaller number of non-cleavable retargeted monomers combined with more VSV G-QQ monomers may be necessary for fusion and entry to occur (FIG. 64).

### Example 41. In Vivo Targeting of an EGFR+ Tumor by VSV Displaying EGF, Administered Intravenously in a SCID Mouse Model

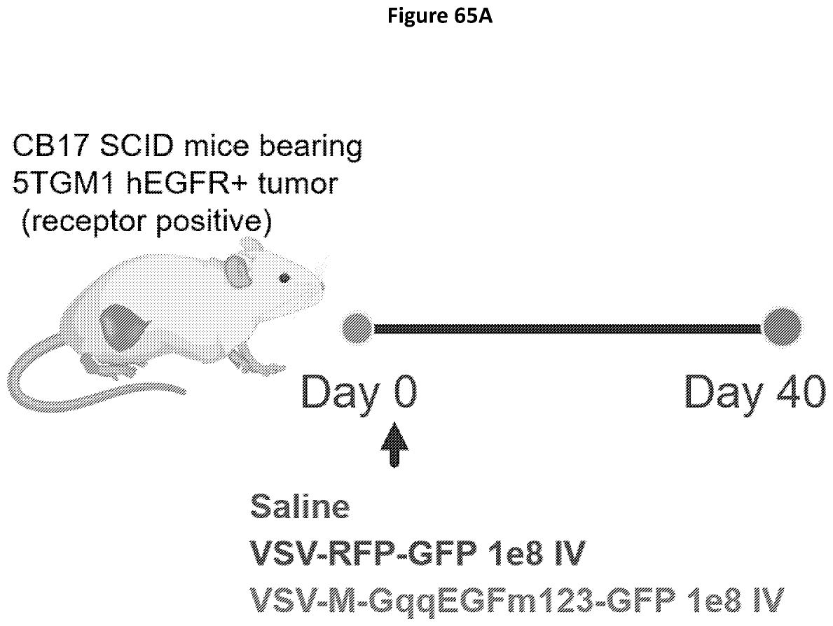

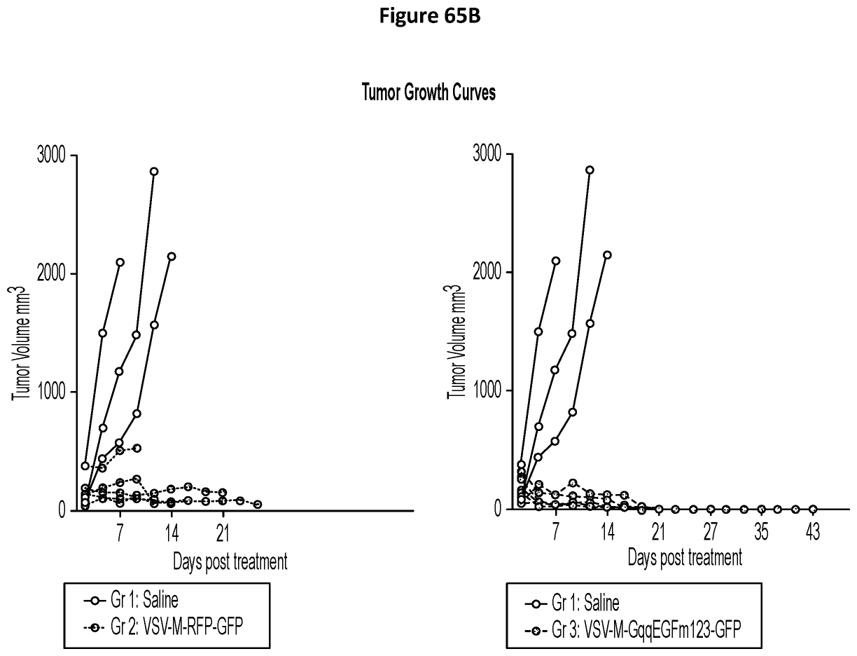

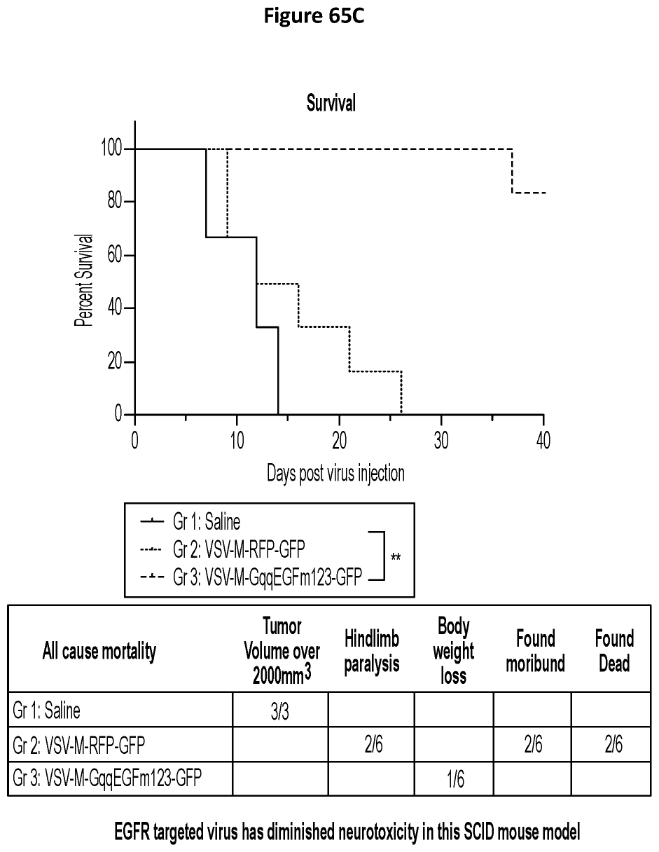

Female SCID mice were implanted with murine myeloma cells (5TGM1) expressing human EGF receptor. Once the tumors reached approximately 150 mm³, the mice were divided into treatment groups and administered either saline, untargeted VSV-M-RFP-GFP, or EGFR-targeted VSV-M-GqqEGFm123-GFP intravenously (FIG. 65A). The mice were monitored over 40 days for clinical signs, body weight changes, and tumor growth (FIGS. 65A-65B). The results demonstrated stark differences between the untargeted and targeted VSV groups. Mice treated with untargeted VSV experienced severe adverse effects, with no mice surviving past day 21 post-infection. In sharp contrast, the EGFR-targeted VSV completely arrested tumor growth in all treated mice, with no adverse effects observed over the 40-day study (FIGS. 65B-65C).

### Example 42. Optimization of the Cytoplasmic Tail of KRV-G

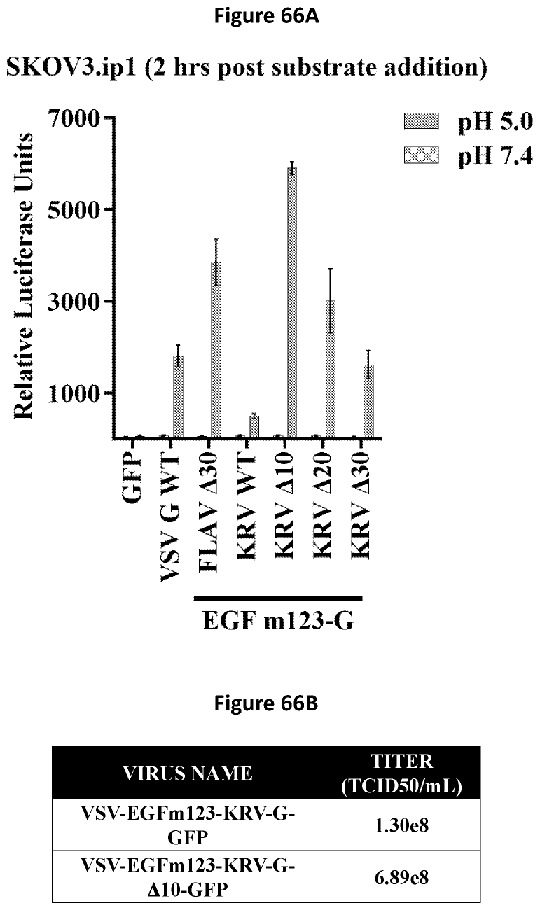

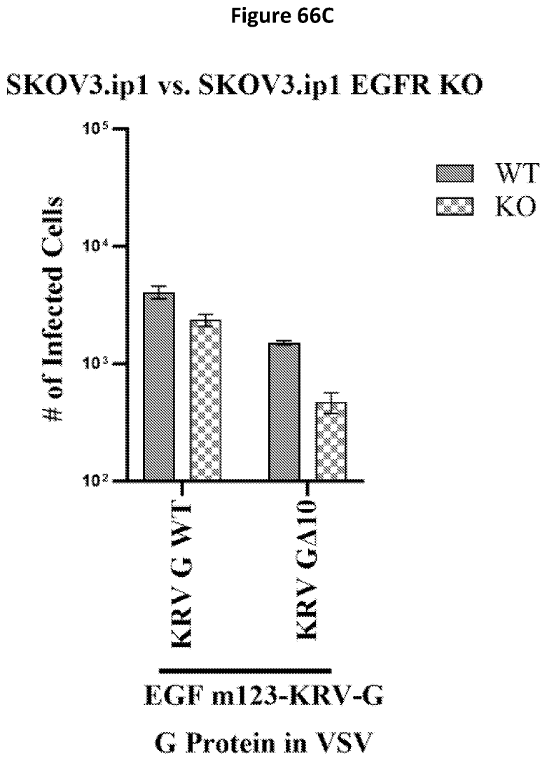

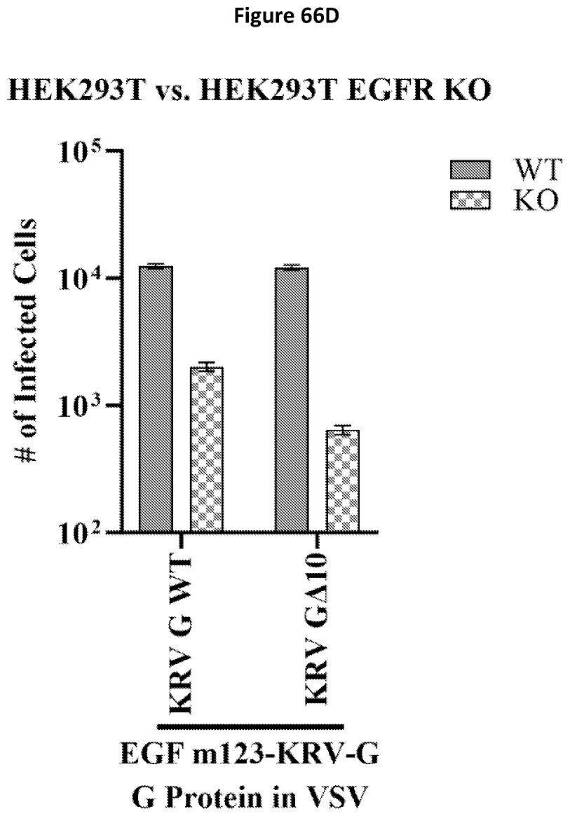

Glycoproteins that performed best in the cell-cell fusion assay (see, e.g., FIG. 37) and the lentivirus pseudotype assay (see, e.g., FIG. 38) were selected for further testing in the context of VSV. The top performing glycoprotein from the cell-cell fusion assay, KRV-G, was first selected for cytoplasmic tail optimization. Truncation of the cytoplasmic tail is an approach which can be used to optimize incorporation into a heterologous viral vector and may increase fusion activity. To identify the optimal cytoplasmic tail length, a series of deletions mutants increasing in size by 10 amino acids was generated for KRV-G. These plasmids were used to test in a cell-cell fusion assay where KRV-G with the C-terminal 10 amino acids removed (Δ10) had substantially increased fusion activity relative to the WT KRV-G (FIG. 66A). To test whether this optimization was also an improvement in the context of replicative VSV, GFP-expressing VSVs were generated with KRV-G WT or A10 that contained N-terminal EGFm123. After rescue and amplification of the viruses, titration on Vero cells stably expressing human EGFR revealed a 5-fold increase in titer of the A10 KRV-G VSV compared to VSV with the full length KRV G (FIG. 66B). The specificity of these two VSVs was also compared on panel of SKOV3.ip1 cells (FIG. 66C) or HEK293T (FIG. 66D) cells naturally expressing EGFR or with that gene knocked out (KO). Surprisingly, the KRV-G A10-containing VSV exhibited an increase in specificity compared to VSV with WT KRV-G. Collectively, the optimized KRV-G enhanced the fitness of the virus, as well as the specificity in the context of VSV.

### Example 43. Sensitivity of VSVs Pseudotyped with Other Rhabdovirus Glycoproteins to Serum Complement

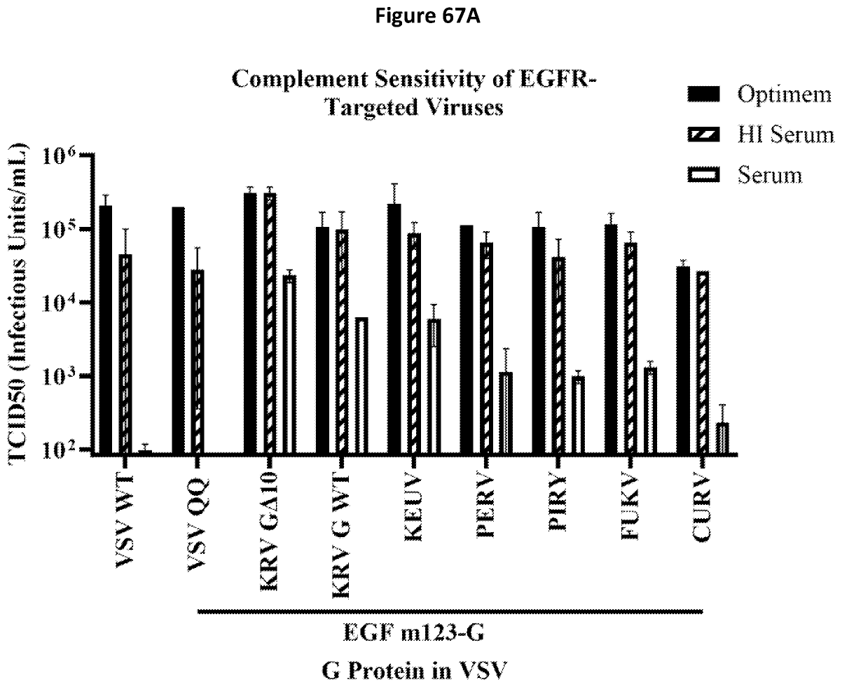

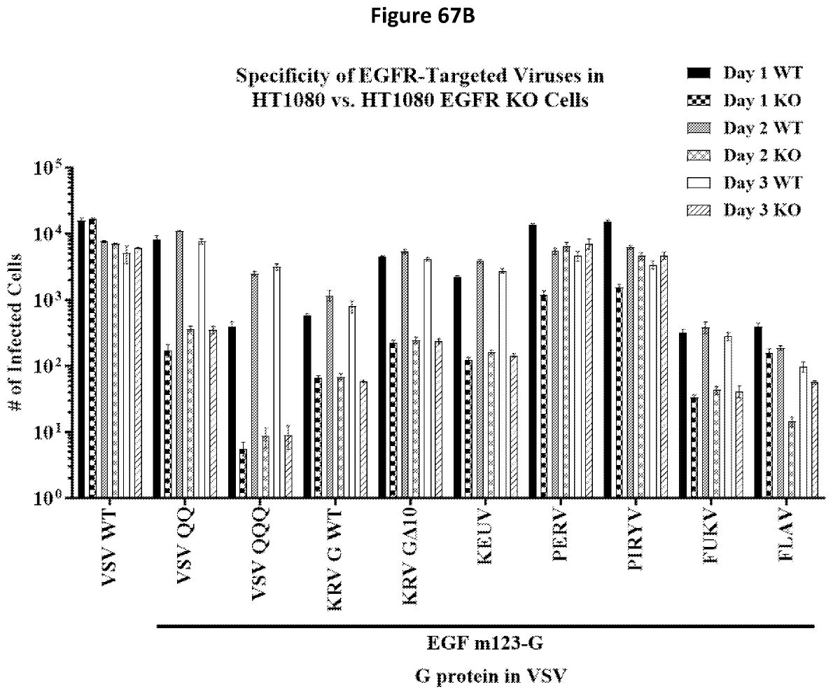

To investigate the sensitivity of VSVs pseudotyped with other rhabdovirus glycoproteins to serum complement, replicative GFP-expressing VSVs containing some of the top-performing alternative rhabdovirus glycoproteins and an N-terminal EGFm123 molecule were generated. These recombinant viruses were tested in a serum sensitivity assay in which the viruses were treated with OptiMEM, serum that had been heat inactivated, or complement-active serum. VSVs with alternative glycoproteins exhibited varying degrees of resistance to complement-mediated degradation but were all superior to VSV-G (FIG. 67A). KRV-G exhibited the highest level of resistance. The specificity of the same set of viruses was also tested on HT1080 WT or EGFR knock out cells (FIG. 67B). VSVs pseudotyped with glycoproteins from the lendantevirus genus exhibited a high level of specificity for the EGFR target even though no mutations had been made to ablate binding to the glycoprotein's natural receptor. The specificity, particularly for KRV-G and KEUV-G, was better than VSV G containing two mutations (QQ) to ablate receptor binding. There was limited specificity for EGFR of the PERV-G and PIRYV-G pseudotyped VSVs in this cell type. These glycoproteins are from viruses that fall within the same vesiculovirus genus as VSV, and thus may require mutations to ablate, e.g., LDLR interaction, similar to VSV G.

### Example 44. Redirected KRV-G-Mediated Virus Entry Using Single-Chain Variable Fragments (scFvs)

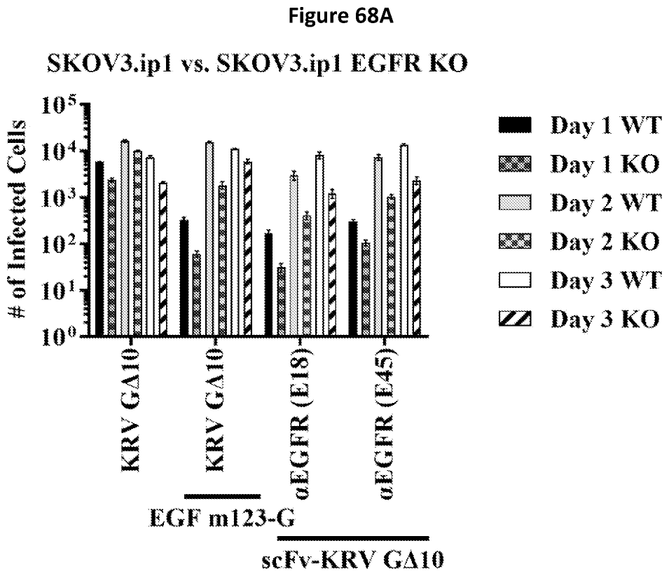

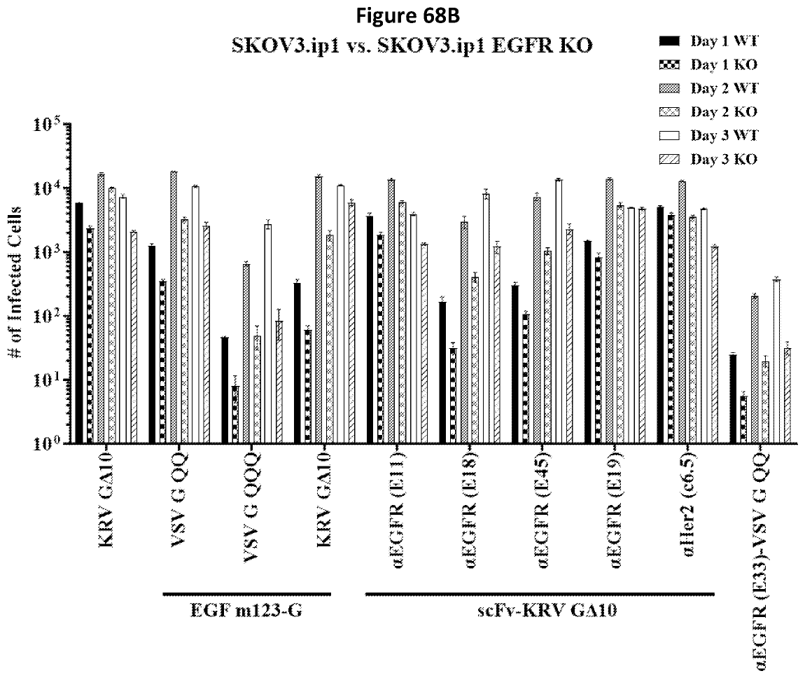

To determine whether KRV-G-mediated virus entry can also be redirected using single-chain variable fragments (scFvs), several KRV-G pseudotyped VSVs were generated with one of several anti-EGF receptor (EGFR) scFvs, or an anti-Her2 scFv, and compared to the EGFm123-modified KRV-G (see, e.g., FIGS. 68A-68B). Non-targeted (i.e., no N-terminal targeting moiety) and the indicated VSV-G containing VSVs were used as controls. These modified rhabdoviruses were applied to SKOV3.ip1 cells (SKOV3.ip1 WT, EGFR-expressing) and SKOV3.ip1 cells wherein EGFR was knocked out (SKOV3.ip1 KO). The total number of infected cells were quantified to demonstrate both the overall infective potential and the specificity (output from the SKOV3.ip1 WT populations compared to SKOV3.ip1 KO), and were compared to results from blinded and retargeted VSV platforms (G QQ and G QQQ with EGFm123) and KRV-G A10 with a targeting addition that is not relevant to the exposed cells (anti-Her2) (FIG. 68B).

Although KRV-G A10 was still capable of entering SKOV3.ip1 cells without the addition of targeting moieties, the addition of an anti-EGFR target conferred a high degree of specificity (FIG. 68B). For each of the represented rhabdovirus modifications containing an EGFR scFv, the number of infected SKOV3.ip1 WT cells was significantly greater than the number of infected SKOV3.ip1 KO cells. This trend was maintained across the three-day measurement window for this experiment. Furthermore, the best-performing EGFR-targeted KRV-G A10 viruses (using anti-EGFR scFvs E18 and E45) are as specifically targeted as blinded and retargeted VSV-G. Anti-EGFR targeted KRV-G A10 is tractable and confers target-specific homing to cells of interest.

### Example 45. Design and Testing of VSV-G Protein Deletion Mutants

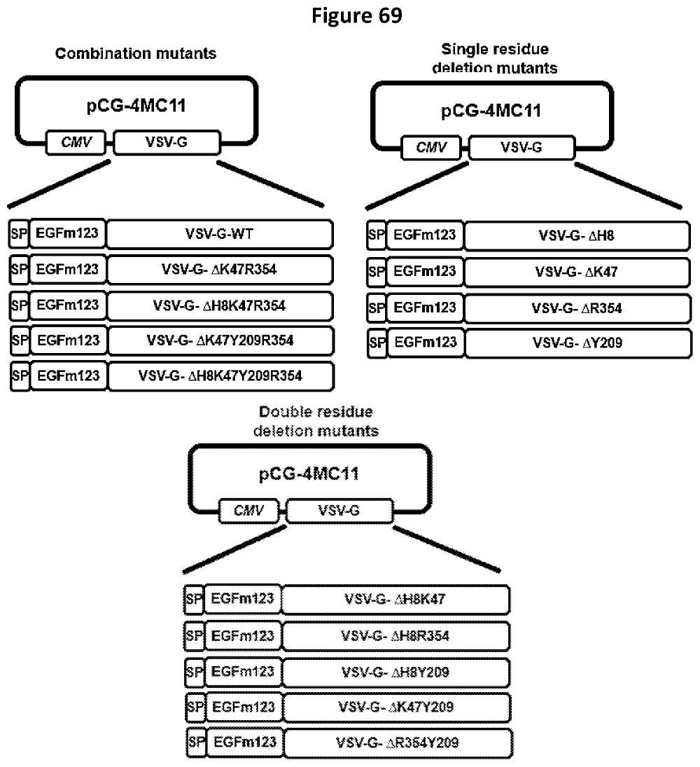

Examples of designs of retargeted mutant VSV-G constructs tested in the present Example are shown in FIG. 69. The VSV-G protein contained a signal peptide (SP) that was proteolytically cleaved following translation. The targeting molecule was a natural ligand such as, but not limited to, EGFm123 (modified EGF) which could be appended to the amino-terminus of the G protein, e.g., without a flexible linker. The LDLR binding residues (H8, K47, R354 and Y209) were deleted in the G protein either in single, double, triple or quadruple combinations.

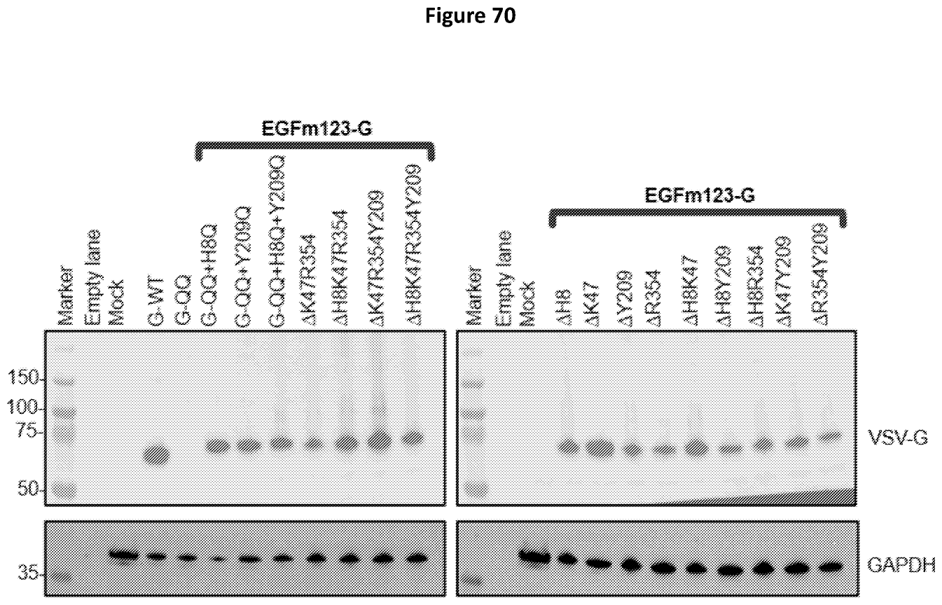

Plasmid DNA constructs were transfected along with lentiviral transfer and packaging constructs to produce the lentivirus in the HEK-293T cells. Substitution mutants, triple or quadruple, combined with G-QQ (K47QR354Q), were included for comparison. After the collection of lentivirus supernatants, cell lysates were collected at 72 hours post-transfection, followed by western blotting with anti-VSV-G and anti-GAPDH antibodies (FIG. 70). The data indicated that the deletion of residues in the VSV-G protein did not affect protein expression in the cells.

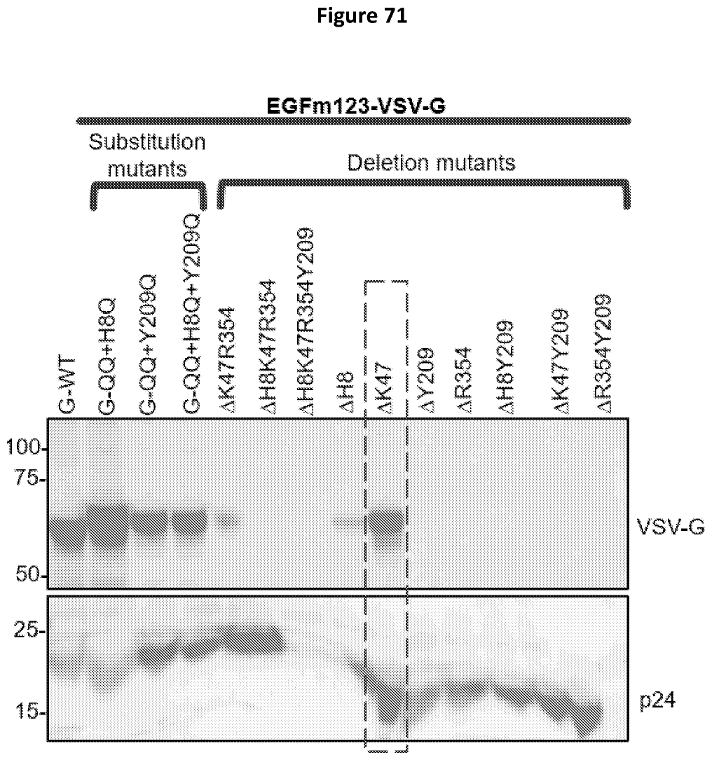

Lentivirus supernatants collected at 72 hours post-transfection were titered by a p24 ELISA method. A total of 5×10⁵ physical particles were lysed and loaded onto SDS-PAGE, followed by Western blotting with anti-VSV-G and p24 antibodies (FIG. 71). The data showed that although protein expression was not affected, only the K47 residue deletion mutant showed better incorporation of chimeric protein into the lentivirus particle which is comparable to the substitution mutants.

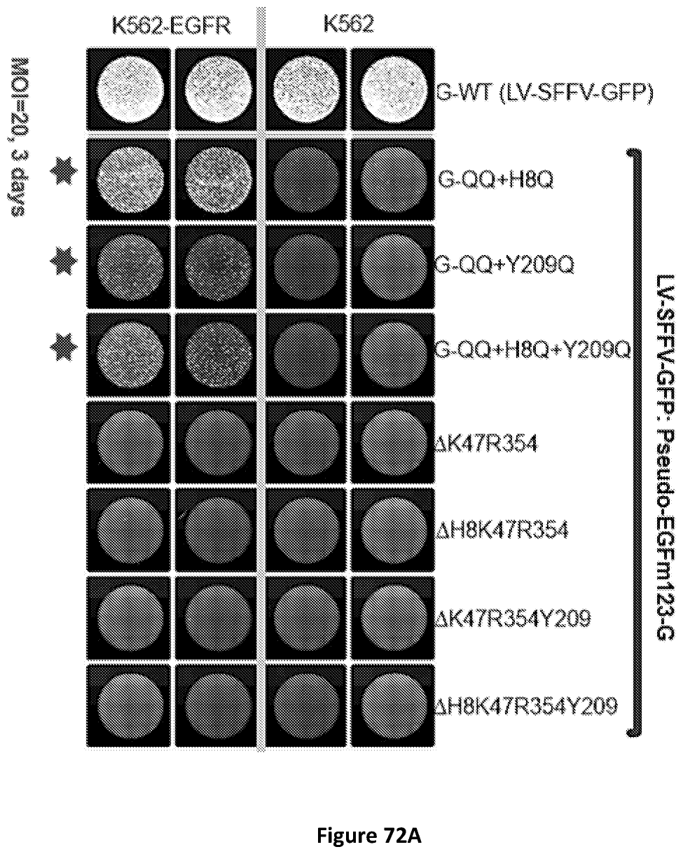

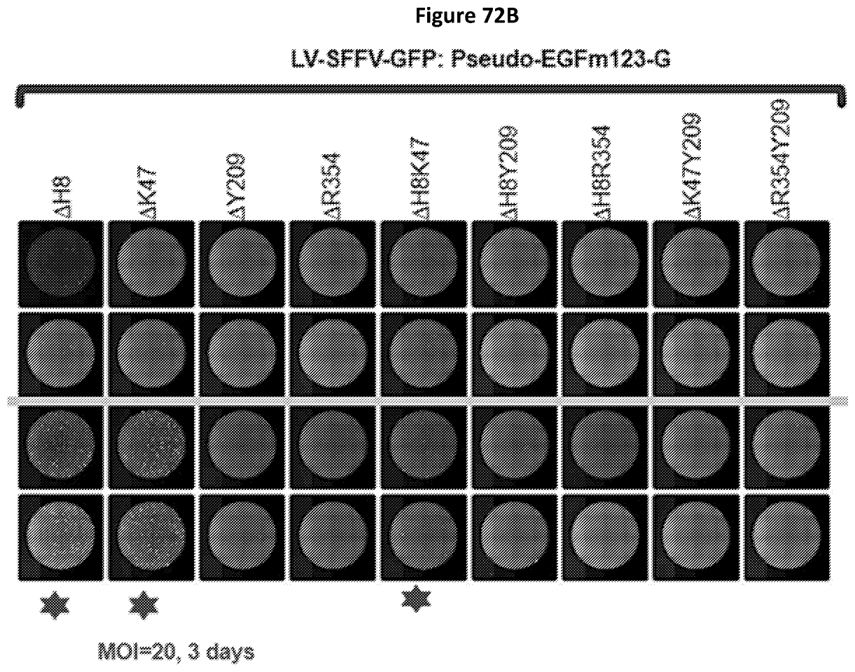

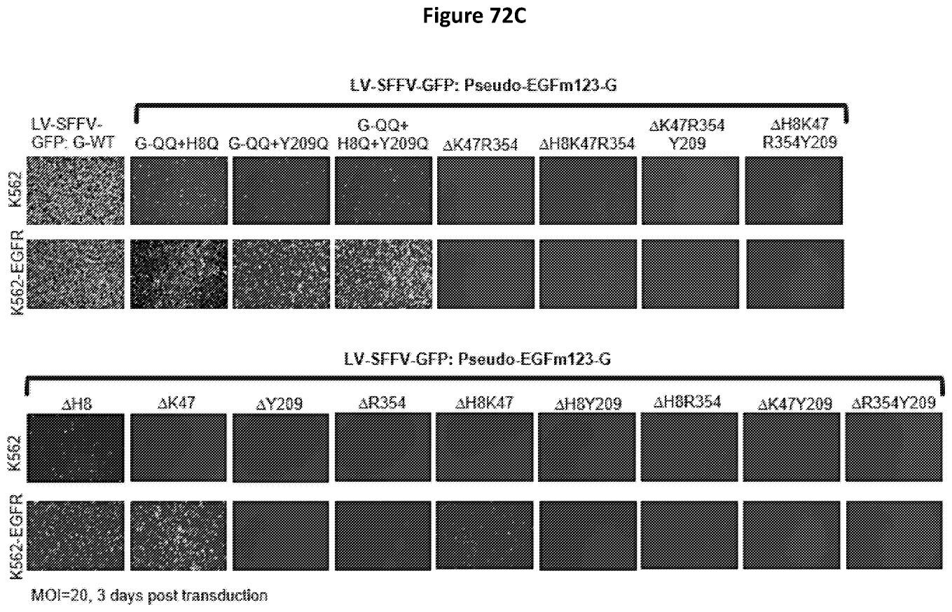

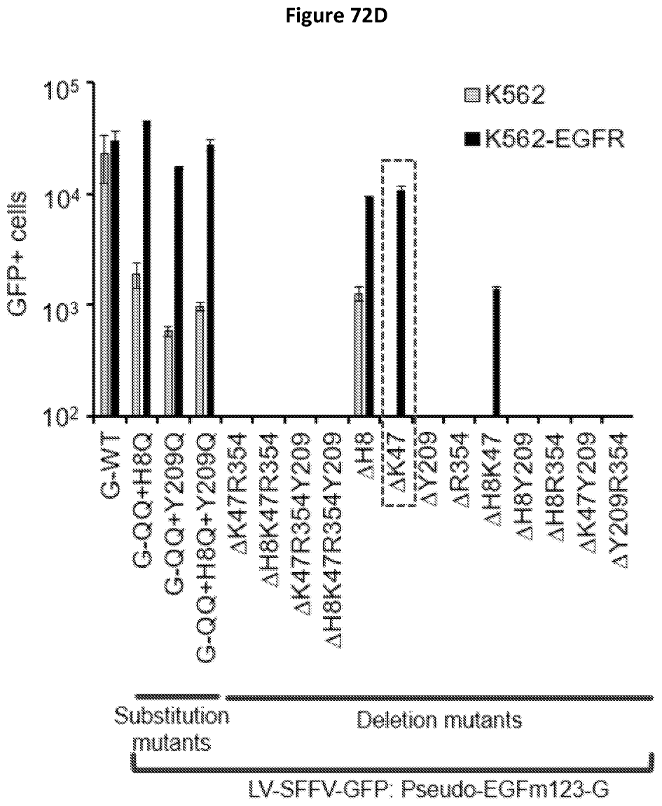

Lentivirus transduction on the K562 cell panel is shown in FIGS. 72A-72B. K562 parental or K562-EGFR cells were transduced with the indicated retargeted lentiviruses displaying EGFm123 at a MOI=20. Lentivirus pseudotyped with WT-G was used as a control. Celigo images (FIGS. 72A-72B) and Nikon images (FIG. 72C) were taken at 72 hours post-transduction and transduced GFP positive cells were quantified (FIG. 72D). The data showed that lentivirus pseudotyped with WT-G transduced both K562 parental and K56-EGFR cells comparably, whereas substitution mutants specifically transduced K562-EGFR cells with minimal background in the K562 parental cells. Among the deletion mutants, retargeted lentiviruses with deletion of K47 residues showed efficient and specific transduction on K562-EGFR cells, whereas deletion H8 or double deletion of H8 and K47 residues showed retargeting specificity with minimal background. These data indicated that VSV-G can be detargeted from its binding to natural receptor by deletion of K47, and can be retargeted to receptor of interest.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

1. A recombinant fusogenic protein, wherein said fusogenic protein comprises:
(i) a rhabdovirus glycoprotein (G), or a functional fragment or derivative thereof; and
(ii) a targeting molecule, wherein said targeting molecule is attached to the N-terminus of said rhabdovirus glycoprotein, or the functional fragment or derivative thereof, via a linker, said linker being sensitive to a proteolytic cleavage by an endogenous protease or by an exogenously added protease.

2. The recombinant fusogenic protein of claim 1, wherein said linker comprises an Arginine (R) and/or Lysine (K) residue.

3. The recombinant fusogenic protein of claim 1, wherein said linker is comprised within the sequence selected from:
(SEQ ID NO: 174)
KRAAASGGS(G₄S)₂GPK;
(SEQ ID NO: 2)
KRAAASGGS(G₄S)₂;
(SEQ ID NO: 3)
(EAAAK)₃;
(SEQ ID NO: 4)
KR(EAAAK)₃;
(SEQ ID NO: 5)
AAARGSPK(G₄S)₃;
(SEQ ID NO: 169)
RAAARGSPK(G₄S)₃;
(SEQ ID NO: 19)
AAARGSPK(G₄S)₃K;
(SEQ ID NO: 20)
K(G₄S)₃;
(SEQ ID NO: 21)
KR(G₄S)₃;
(SEQ ID NO: 6)
(G₄S)₃GPK;
and
(SEQ ID NO: 7)
AAA(G₄S)₃K.

4. The recombinant fusogenic protein of any one of claims 1-3, wherein the N-terminus of the rhabdovirus glycoprotein, or the functional fragment or derivative thereof, to which the targeting molecule is attached, via a linker, does not comprise one or more amino acids present at the N-terminus of a mature wild-type rhabdovirus glycoprotein.

5. The recombinant fusogenic protein of any one of claims 1-4, wherein said rhabdovirus glycoprotein is a vesicular stomatitis virus glycoprotein (VSV-G), or a functional fragment or derivative thereof.

6. The recombinant fusogenic protein of claim 5, wherein said VSV-G comprises the sequence SEQ ID NO: 8.

7. The recombinant fusogenic protein of claim 6, wherein said VSV-G consists of the sequence SEQ ID NO: 8.

8. The recombinant fusogenic protein of any one of claims 5-7, wherein the targeting molecule is capable of interfering with the ability of said VSV-G, or the functional fragment or derivative thereof, to interact with low-density lipoprotein receptor (LDLR).

9. The recombinant fusogenic protein of any one of claims 5-8, wherein said VSV-G, or the functional fragment or derivative thereof, comprises one or more mutations, wherein the one or more mutations reduces or eliminates binding of said VSV-G polypeptide, or the functional fragment or derivative thereof, to LDLR.

10. The recombinant fusogenic protein of claim 9, wherein said one or more mutations in said VSV-G, or the functional fragment or derivative thereof, comprise one or more amino acid substitutions and/or deletions at positions corresponding to H8, K47, Y209, or R354 in SEQ ID NO: 8.

11. The recombinant fusogenic protein of claim 10, wherein said VSV-G comprises or consists of SEQ ID NO: 8, and said one or more mutations are substitutions at positions K47 and R354.

12. The recombinant fusogenic protein of claim 10, wherein said VSV-G comprises or consists of SEQ ID NO: 8, and said one or more mutations are substitutions at positions K47, R354 and Y209.

13. The recombinant fusogenic protein of claim 10, wherein said VSV-G comprises or consists of SEQ ID NO: 8, and said one or more mutations is a substitution at position H8.

14. The recombinant fusogenic protein of claim 10, wherein said one or more mutations in said VSV-G, or the functional fragment or derivative thereof, comprise one or more amino acid deletions at positions corresponding to H8, K47, Y209, or R354 in SEQ ID NO: 8.

15. The recombinant fusogenic protein of claim 14, wherein said VSV-G comprises or consists of SEQ ID NO: 8.

16. The recombinant fusogenic protein of claim 15, wherein said one or more deletions is a deletion at position K47.

17. The recombinant fusogenic protein of claim 15, wherein said one or more deletions is a deletion at position H8.

18. The recombinant fusogenic protein of claim 15, wherein said one or more deletions are deletions at positions H8 and K47.

19. The recombinant fusogenic protein of any one of claims 5-18, wherein said VSV-G, or the functional fragment or derivative thereof, further comprises one or more viral titer increasing mutations.

20. The recombinant fusogenic protein of claim 19, wherein said one or more viral titer increasing mutations in said VSV-G, or the functional fragment or derivative thereof, is M184T and/or F250L, as specified relative to positions in SEQ ID NO: 8.

21. The recombinant fusogenic protein of any one of claims 1-4, wherein said rhabdovirus glycoprotein is a glycoprotein from Flanders virus (FLAV-G).

22. The recombinant fusogenic protein of claim 21, wherein said FLAV-G comprises the sequence SEQ ID NO: 9.

23. The recombinant fusogenic protein of claim 22, wherein said FLAV-G consists of the sequence SEQ ID NO: 9.

24. The recombinant fusogenic protein of any one of claims 1-4, wherein said rhabdovirus glycoprotein is a glycoprotein from Chandipura virus (CHPV-G).

25. The recombinant fusogenic protein of claim 24, wherein said CHPV-G comprises the sequence SEQ ID NO: 10.

26. The recombinant fusogenic protein of claim 25, wherein said CHPV-G consists of the sequence SEQ ID NO: 10.

27. The recombinant fusogenic protein of any one of claims 1-4, wherein said rhabdovirus glycoprotein is a glycoprotein from Perinet virus (PERV-G).

28. The recombinant fusogenic protein of claim 27, wherein said PERV-G comprises the sequence SEQ ID NO: 11.

29. The recombinant fusogenic protein of claim 28, wherein said PERV-G consists of the sequence SEQ ID NO: 11.

30. The recombinant fusogenic protein of any one of claims 1-4, wherein said rhabdovirus glycoprotein is a glycoprotein from Piry virus (PIRYV-G).

31. The recombinant fusogenic protein of claim 30, wherein said PIRYV-G comprises the sequence SEQ ID NO: 12.

32. The recombinant fusogenic protein of claim 31, wherein said PIRYV-G consists of the sequence SEQ ID NO: 12.

33. The recombinant fusogenic protein of any one of claims 1-4, wherein said rhabdovirus glycoprotein is a glycoprotein from Fukuoka virus (FUKV-G).

34. The recombinant fusogenic protein of claim 33, wherein said FUKV-G comprises the sequence SEQ ID NO: 13.

35. The recombinant fusogenic protein of claim 34, wherein said FUKV-G consists of the sequence SEQ ID NO: 13.

36. The recombinant fusogenic protein of any one of claims 1-4, wherein said rhabdovirus glycoprotein is a glycoprotein from Joinjakaka virus (JOIV-G).

37. The recombinant fusogenic protein of claim 36, wherein said JOIV-G comprises the sequence SEQ ID NO: 14.

38. The recombinant fusogenic protein of claim 37, wherein said JOIV-G consists of the sequence SEQ ID NO: 14.

39. The recombinant fusogenic protein of any one of claims 1-4, wherein said rhabdovirus glycoprotein is a glycoprotein from Kumasi virus (KRV-G).

40. The recombinant fusogenic protein of claim 39, wherein said KRV-G comprises the sequence SEQ ID NO: 15.

41. The recombinant fusogenic protein of claim 40, wherein said KRV-G consists of the sequence SEQ ID NO: 15.

42. The recombinant fusogenic protein of any one of claims 1-4, wherein said rhabdovirus glycoprotein is a glycoprotein from Keuraliba virus (KEUV-G).

43. The recombinant fusogenic protein of claim 42, wherein said KEUV-G comprises the sequence SEQ ID NO: 17.

44. The recombinant fusogenic protein of claim 43, wherein said KEUV-G comprises the sequence SEQ ID NO: 17.

45. The recombinant fusogenic protein of any one of claims 21-44, wherein the cytoplasmic tail of the rhabdovirus glycoprotein has been removed or truncated, and optionally replaced with another sequence.

46. The recombinant fusogenic protein of claim 45, wherein the cytoplasmic tail of the glycoprotein is truncated by up to 40 amino acids from the C-terminus.

47. The recombinant fusogenic protein of claim 46, wherein the cytoplasmic tail of the rhabdovirus glycoprotein is truncated by 10 to 40 amino acids from the C-terminus.

48. The recombinant fusogenic protein of claim 47, wherein the cytoplasmic tail of the rhabdovirus glycoprotein is truncated by 30 amino acids from the C-terminus.

49. The recombinant fusogenic protein of any one of claims 45-48, further comprising a cytoplasmic tail from VSV-G, or a functional fragment or derivative thereof.

50. The recombinant fusogenic protein of claim 49, wherein the cytoplasmic tail of VSV-G comprises the sequence CIKLKHTKKRQIYTDIEMNRLGK (SEQ ID NO: 16).

51. A recombinant fusogenic protein, wherein said fusogenic protein comprises a fusogen that has at least 60% amino acid sequence identity to a vesicular stomatitis virus glycoprotein (VSV-G) comprising SEQ ID NO: 8, or a functional fragment or derivative thereof, wherein said fusogen, or the functional fragment or derivative thereof, comprises one or more amino acid deletions at positions corresponding to H8, K47, Y209, or R354 in SEQ ID NO: 8.

52. The recombinant fusogenic protein of claim 51, wherein said fusogen comprises the sequence SEQ ID NO: 8, or the functional fragment or derivative thereof, with one or more amino acid deletions at positions H8, K47, Y209, or R354.

53. The recombinant fusogenic protein of claim 52, wherein said fusogen comprises or consists of the sequence SEQ ID NO: 8, with an amino acid deletion at position H8.

54. The recombinant fusogenic protein of claim 52, wherein said fusogen comprises or consists of the sequence SEQ ID NO: 8, with amino acid deletions at positions H8 and K47.

55. The recombinant fusogenic protein of claim 52, wherein said fusogen comprises the sequence SEQ ID NO: 8, with amino acid deletions at positions (i) K47, (ii) R354, and (iii) H8 or Y209.

56. The recombinant fusogenic protein of claim 55, wherein said fusogen consists of the sequence SEQ ID NO: 8, with amino acid deletions at positions (i) K47, (ii) R354, and (iii) H8 or Y209.

57. The recombinant fusogenic protein of claim 52, wherein said fusogen comprises the sequence SEQ ID NO: 8, with an amino acid deletion at position K47.

58. The recombinant fusogenic protein of claim 57, wherein said fusogen consists of the sequence SEQ ID NO: 8, with an amino acid deletion at positions K47.

59. A recombinant fusogenic protein that comprises a fusogen that comprises the sequence SEQ ID NO: 8, with amino acid substitutions at positions (i) K47, (ii) R354, and (iii) H8 or Y209.

60. The recombinant fusogenic protein of claim 59, wherein said fusogen consists of the sequence SEQ ID NO: 8, with amino acid substitutions at positions (i) K47, (ii) R354, and
(iii) H8 or Y209.

61. A recombinant fusogenic protein that comprises a fusogen that comprises the sequence SEQ ID NO: 8, with amino acid substitutions at positions K47, R354, H8, and Y209.

62. The recombinant fusogenic protein of claim 61, wherein said fusogen consists of the sequence SEQ ID NO: 8, with amino acid substitutions at positions K47, R354, H8, and Y209.

63. The recombinant fusogenic protein of any one of claims 51-62, wherein said fusogen, or the functional fragment or derivative thereof, further comprises one or more viral titer increasing mutations.

64. The recombinant fusogenic protein of claim 63, wherein said one or more viral titer increasing mutations are in one or more positions corresponding to positions M184 and/or F250 in SEQ ID NO: 8.

65. The recombinant fusogenic protein of any one of claims 51-64, further comprising a targeting molecule located at the N-terminus of said fusogen, or the functional fragment or derivative thereof.

66. The recombinant fusogenic protein of claim 65, wherein said targeting molecule is attached to the N-terminus of said fusogen, or the functional fragment or derivative thereof, via a linker.

67. The recombinant fusogenic protein of claim 66, wherein said linker is sensitive to a proteolytic cleavage by an endogenous protease or by an exogenously added protease.

68. The recombinant fusogenic protein of claim 67, wherein said linker comprises an Arginine (R) and/or Lysine (K) residue.

69. The recombinant fusogenic protein of claim 67, wherein said linker is comprised within the sequence selected from:
(SEQ ID NO: 174)
KRAAASGGS(G₄S)₂GPK;
(SEQ ID NO: 2)
KRAAASGGS(G₄S)₂;
(SEQ ID NO: 3)
(EAAAK)₃;
(SEQ ID NO: 4)
KR(EAAAK)₃;
(SEQ ID NO: 5)
AAARGSPK(G₄S)₃;
(SEQ ID NO: 169)
RAAARGSPK(G₄S)₃;
(SEQ ID NO: 19)
AAARGSPK(G₄S)₃K;
(SEQ ID NO: 20)
K(G₄S)₃;
(SEQ ID NO: 21)
KR(G₄S)₃;
(SEQ ID NO: 6)
(G₄S)₃GPK;
and
(SEQ ID NO: 7)
AAA(G₄S)₃K.

70. The recombinant fusogenic protein of claim 66, wherein said linker is not sensitive to a proteolytic cleavage by an endogenous protease or by an exogenously added protease.

71. The recombinant fusogenic protein of any one of claims 65-70, wherein the N-terminus of the fusogen, or the functional fragment or derivative thereof, to which the targeting molecule is attached, does not comprise one or more amino acids present at the N-terminus of a mature wild-type fusogen.

72. A recombinant fusogenic protein, wherein said fusogenic protein comprises:
(i) a glycoprotein from Flanders virus (FLAV-G), or a functional fragment or derivative thereof, and
(ii) a targeting molecule.

73. The recombinant fusogenic protein of claim 72, wherein said FLAV-G comprises the sequence SEQ ID NO: 9.

74. The recombinant fusogenic protein of claim 73, wherein said FLAV-G consists of the sequence SEQ ID NO: 9.

75. A recombinant fusogenic protein, wherein said fusogenic protein comprises:
(i) a glycoprotein from Chandipura virus (CHPV-G), or a functional fragment or derivative thereof, and
(ii) a targeting molecule.

76. The recombinant fusogenic protein of claim 75, wherein said CHPV-G comprises the sequence SEQ ID NO: 10.

77. The recombinant fusogenic protein of claim 76, wherein said CHPV-G consists of the sequence SEQ ID NO: 10.

78. A recombinant fusogenic protein, wherein said fusogenic protein comprises:
(i) a glycoprotein from Perinet virus (PERV-G), or a functional fragment or derivative thereof, and
(ii) a targeting molecule.

79. The recombinant fusogenic protein of claim 78, wherein said PERV-G comprises the sequence SEQ ID NO: 11.

80. The recombinant fusogenic protein of claim 79, wherein said PERV-G consists of the sequence SEQ ID NO: 11.

81. A recombinant fusogenic protein, wherein said fusogenic protein comprises:
(i) a glycoprotein from Piry virus (PIRYV-G), or a functional fragment or derivative thereof, and
(ii) a targeting molecule.

82. The recombinant fusogenic protein of claim 81, wherein said PIRYV-G comprises the sequence SEQ ID NO: 12.

83. The recombinant fusogenic protein of claim 82, wherein said PIRYV-G consists of the sequence SEQ ID NO: 12.

84. A recombinant fusogenic protein, wherein said fusogenic protein comprises:
(i) a glycoprotein from Fukuoka virus (FUKV-G), or a functional fragment or derivative thereof, and
(ii) a targeting molecule.

85. The recombinant fusogenic protein of claim 84, wherein said FUKV-G comprises the sequence SEQ ID NO: 13.

86. The recombinant fusogenic protein of claim 85, wherein said FUKV-G consists of the sequence SEQ ID NO: 13.

87. A recombinant fusogenic protein, wherein said fusogenic protein comprises:
(i) a glycoprotein from Joinjakaka virus (JOIV-G), or a functional fragment or derivative thereof, and
(ii) a targeting molecule.

88. The recombinant fusogenic protein of claim 87, wherein said JOIV-G comprises the sequence SEQ ID NO: 14.

89. The recombinant fusogenic protein of claim 88, wherein said JOIV-G consists of the sequence SEQ ID NO: 14.

90. A recombinant fusogenic protein, wherein said fusogenic protein comprises:
(i) a glycoprotein from Kumasi virus (KRV-G), or a functional fragment or derivative thereof, and
(ii) a targeting molecule.

91. The recombinant fusogenic protein of claim 90, wherein said KRV-G comprises the sequence SEQ ID NO: 15.

92. The recombinant fusogenic protein of claim 91, wherein said KRV-G consists of the sequence SEQ ID NO: 15.

93. A recombinant fusogenic protein, wherein said fusogenic protein comprises:
(i) a glycoprotein from Keuraliba virus (KEUV-G), or a functional fragment or derivative thereof, and
(ii) a targeting molecule.

94. The recombinant fusogenic protein of claim 93, wherein said KEUV-G comprises the sequence SEQ ID NO: 17.

95. The recombinant fusogenic protein of claim 94, wherein said KEUV-G consists of the sequence SEQ ID NO: 17.

96. The recombinant fusogenic protein of any one of claims 72-95, wherein said glycoprotein is a fragment, wherein the cytoplasmic tail of the glycoprotein has been removed or truncated, and optionally replaced with another sequence.

97. The recombinant fusogenic protein of claim 96, wherein the cytoplasmic tail of the glycoprotein is truncated by up to 40 amino acids from the C-terminus.

98. The recombinant fusogenic protein of claim 97, wherein the cytoplasmic tail of the glycoprotein is truncated by 10 to 40 amino acids from the C-terminus.

99. The recombinant fusogenic protein of claim 98, wherein the cytoplasmic tail of the glycoprotein is truncated by 30 amino acids from the C-terminus.

100. The recombinant fusogenic protein of any one of claims 96-99, further comprising a cytoplasmic tail from VSV-G, or a functional fragment or derivative thereof.

101. The recombinant fusogenic protein of claim 100, wherein the cytoplasmic tail of VSV-G comprises the sequence CIKLKHTKKRQIYTDIEMNRLGK (SEQ ID NO: 16).

102. The recombinant fusogenic protein of any one of claims 72-101, wherein the targeting molecule is located at the N-terminus of said glycoprotein, or the functional fragment or derivative thereof.

103. The recombinant fusogenic protein of claim 102, wherein said targeting molecule is attached to the N-terminus of said glycoprotein, or the functional fragment or derivative thereof, via a linker.

104. The recombinant fusogenic protein of claim 103, wherein said linker is sensitive to a proteolytic cleavage by an endogenous protease or by an exogenously added protease.

105. The recombinant fusogenic protein of claim 104, wherein said linker comprises an Arginine (R) and/or Lysine (K) residue.

106. The recombinant fusogenic protein of claim 104, wherein said linker is comprised within the sequence selected from:
(SEQ ID NO: 174)
KRAAASGGS(G₄S)₂GPK;
(SEQ ID NO: 2)
KRAAASGGS(G₄S)₂;
(SEQ ID NO: 3)
(EAAAK)₃;
(SEQ ID NO: 4)
KR(EAAAK)₃;
(SEQ ID NO: 5)
AAARGSPK(G₄S)₃;
(SEQ ID NO: 169)
RAAARGSPK(G₄S)₃;
(SEQ ID NO: 19)
AAARGSPK(G₄S)₃K;
(SEQ ID NO: 20)
K(G₄S)₃;
(SEQ ID NO: 21)
KR(G₄S)₃;
(SEQ ID NO: 6)
(G₄S)₃GPK;
and
(SEQ ID NO: 7)
AAA(G₄S)₃K.

107. The recombinant fusogenic protein of claim 103, wherein said linker is not sensitive to a proteolytic cleavage by an endogenous protease or by an exogenously added protease.

108. The recombinant fusogenic protein of any one of claims 102-107, wherein the N-terminus of the glycoprotein, or the functional fragment or derivative thereof, to which the targeting molecule is attached, does not comprise one or more amino acids present at the N-terminus of a mature wild-type fusogen.

109. The recombinant fusogenic protein of any one of claims 1-50 and 65-108, wherein said targeting molecule is an antibody or antigen-binding fragment thereof, an affibody, a darpin, a peptide, a natural or modified natural receptor ligand, a T cell receptor or a fragment or derivative thereof, or an MHC-peptide complex or a fragment or derivative thereof.

110. The recombinant fusogenic protein of claim 109, wherein said antibody or antigen-binding fragment thereof is a single-chain fragment variable (scFv), a diabody, a minibody, a nanobody, a single-domain antibody (sdAb), or a single heavy chain antibody.

111. The recombinant fusogenic protein of any one of claims 1-50 and 65-110, wherein said targeting molecule targets EGFR, HER2, MUC16, cKit, αVβ3 Integrin, IGF1R, BCMA, Nectin-4, MEK, CD44, CD3, CD4, CD28, stem cell factor, thrombopoietin, c-Met, CXCR4, IL2R, or IL-3.

112. A recombinant polynucleotide encoding the recombinant fusogenic protein of any one of claims 1-111.

113. The recombinant polynucleotide of claim 112, wherein the polynucleotide comprises a sequence encoding a signal peptide sequence, wherein such signal sequence is positioned at the extreme N-terminus of the encoded recombinant fusogenic protein.

114. The recombinant polynucleotide of claim 112 or claim 113, wherein the polynucleotide is DNA.

115. The recombinant polynucleotide of claim 112 or claim 113, wherein the polynucleotide is RNA.

116. A recombinant polynucleotide, wherein the recombinant polynucleotide is an RNA molecule comprising a nucleotide sequence that is a template for a positive sense transcript encoding the recombinant fusogenic protein of any one of claims 1-111.

117. The recombinant polynucleotide of claim 116, wherein the positive sense transcript comprises a sequence encoding a signal peptide sequence, wherein such signal sequence is positioned at the extreme N-terminus of the encoded recombinant fusogenic protein.

118. The recombinant polynucleotide of claim 116 or claim 117, wherein the recombinant polynucleotide is an RNA molecule comprising a nucleotide sequence that is a template for a positive sense transcript encoding a vesicular stomatitis virus (VSV) nucleoprotein (N) polypeptide or a functional fragment or derivative thereof, a nucleotide sequence that is a template for a positive sense transcript encoding a VSV phosphoprotein (P) polypeptide or a functional fragment or derivative thereof, a nucleotide sequence that is a template for a positive sense transcript encoding a VSV matrix (M) polypeptide or a functional fragment or derivative thereof, a nucleotide sequence that is a template for a positive sense transcript encoding the fusogenic protein of any one of claims 1-111, and a nucleotide sequence that is a template for a positive sense transcript encoding a VSV large protein (L) polypeptide or a functional fragment or derivative thereof.

119. The recombinant polynucleotide of claim 118, wherein said VSV M polypeptide is a mutant VSV M polypeptide.

120. The recombinant polynucleotide of claim 119, wherein said mutant VSV M polypeptide comprises a mutation at methionine (M) 51.

121. The recombinant polynucleotide of claim 120, wherein said mutation at methionine (M) 51 is a substitution from methionine (M) to arginine (R).

122. The recombinant polynucleotide of any one of claims 112-121, wherein said polynucleotide is optimized for expression in human cells.

123. A composition comprising the recombinant polynucleotide of any one of claims 112-122 and a carrier and/or excipient.

124. A host cell comprising the recombinant polynucleotide of any one of claims 112-122.

125. A recombinant pseudotyped virus or cell-derived nanovesicle comprising the recombinant polynucleotide of any one of claims 112-122.

126. A recombinant pseudotyped virus or cell-derived nanovesicle comprising one or more recombinant fusogenic proteins of any one of claims 1-111.

127. The recombinant pseudotyped virus or cell-derived nanovesicle of claim 126, comprising two or more different recombinant fusogenic proteins of any one of claims 1-111.

128. The recombinant pseudotyped virus or cell-derived nanovesicle of claim 126 or claim 127, wherein said recombinant fusogenic protein forms a chimeric trimer with one or two different fusogenic proteins on the surface of said recombinant pseudotyped virus or cell-derived nanovesicle.

129. The recombinant pseudotyped virus or cell-derived nanovesicle of claim 128, wherein said chimeric trimer comprises (i) at least one fusogenic protein of any one of claims 1-111 and (ii) a fusogenic protein comprising a rhabdoviral glycoprotein, or a functional fragment or derivative thereof, without a targeting molecule.

130. The recombinant pseudotyped virus or cell-derived nanovesicle of claim 129, wherein the fusogenic protein (ii) comprises a fusogen that comprises the sequence SEQ ID NO: 8, with amino acid substitutions and/or deletions at one or more positions selected from K47, R354, H8, and Y209.

131. A recombinant pseudotyped virus or cell-derived nanovesicle comprising a chimeric trimer comprising (i) one or two monomers of a first fusogenic protein, wherein said first fusogenic protein comprises a rhabdovirus glycoprotein, or a functional fragment or derivative thereof, and a targeting molecule, or the functional fragment or derivative thereof, and (ii) one or two monomers of a second fusogenic protein, wherein said second fusogenic protein comprises a rhabdovirus glycoprotein, or a functional fragment or derivative thereof, without a targeting molecule.

132. The recombinant pseudotyped virus or cell-derived nanovesicle of claim 131, wherein in the first fusogenic protein, the targeting molecule is attached to the rhabdovirus glycoprotein via a linker.

133. The recombinant pseudotyped virus or cell-derived nanovesicle of claim 132, wherein said linker is not sensitive to a proteolytic cleavage by an endogenous protease or by an exogenously added protease.

134. The recombinant pseudotyped virus or cell-derived nanovesicle of claim 132, wherein said linker is sensitive to a proteolytic cleavage by an endogenous protease or by an exogenously added protease.

135. The recombinant pseudotyped virus or cell-derived nanovesicle of any one of claims 131-134, wherein the first fusogenic protein and/or the second fusogenic protein comprises a rhabdovirus glycoprotein that comprises the sequence SEQ ID NO: 8, with amino acid substitutions and/or deletions at one or more positions selected from K47, R354, H8, and Y209.

136. The recombinant pseudotyped virus or cell-derived nanovesicle of claim 135, wherein the first fusogenic protein and/or the second fusogenic protein comprises a rhabdovirus glycoprotein that comprises the sequence set forth in any of claims 10-18 or 52-54.

137. A recombinant pseudotyped virus or cell-derived nanovesicle comprising a glycoprotein from Flanders virus (FLAV-G), or a functional fragment or derivative thereof.

138. The recombinant pseudotyped virus or cell-derived nanovesicle of claim 137, wherein said FLAV-G comprises the sequence SEQ ID NO: 9.

139. The recombinant pseudotyped virus or cell-derived nanovesicle of claim 138, wherein said FLAV-G consists of the sequence SEQ ID NO: 9.

140. A recombinant pseudotyped virus or cell-derived nanovesicle comprising a glycoprotein from Chandipura virus (CHPV-G), or a functional fragment or derivative thereof.

141. The recombinant pseudotyped virus or cell-derived nanovesicle of claim 140, wherein said CHPV-G comprises the sequence SEQ ID NO: 10.

142. The recombinant pseudotyped virus or cell-derived nanovesicle of claim 141, wherein said CHPV-G consists of the sequence SEQ ID NO: 10.

143. A recombinant pseudotyped virus or cell-derived nanovesicle comprising a glycoprotein from Perinet virus (PERV-G), or a functional fragment or derivative thereof.

144. The recombinant pseudotyped virus or cell-derived nanovesicle of claim 143, wherein said PERV-G comprises the sequence SEQ ID NO: 11.

145. The recombinant pseudotyped virus or cell-derived nanovesicle of claim 144, wherein said PERV-G consists of the sequence SEQ ID NO: 11.

146. A recombinant pseudotyped virus or cell-derived nanovesicle comprising a glycoprotein from Piry virus (PIRYV-G), or a functional fragment or derivative thereof.

147. The recombinant pseudotyped virus or cell-derived nanovesicle of claim 146, wherein said PIRYV-G comprises the sequence SEQ ID NO: 12.

148. The recombinant pseudotyped virus or cell-derived nanovesicle of claim 147, wherein said PIRYV-G consists of the sequence SEQ ID NO: 12.

149. A recombinant pseudotyped virus or cell-derived nanovesicle comprising a glycoprotein from Fukuoka virus (FUKV-G), or a functional fragment or derivative thereof.

150. The recombinant pseudotyped virus or cell-derived nanovesicle of claim 149, wherein said FUKV-G comprises the sequence SEQ ID NO: 13.

151. The recombinant pseudotyped virus or cell-derived nanovesicle of claim 150, wherein said FUKV-G consists of the sequence SEQ ID NO: 13.

152. A recombinant pseudotyped virus or cell-derived nanovesicle comprising a glycoprotein from Joinjakaka virus (JOIV-G), or a functional fragment or derivative thereof.

153. The recombinant pseudotyped virus or cell-derived nanovesicle of claim 152, wherein said JOIV-G comprises the sequence SEQ ID NO: 14.

154. The recombinant pseudotyped virus or cell-derived nanovesicle of claim 153, wherein said JOIV-G consists of the sequence SEQ ID NO: 14.

155. A recombinant pseudotyped virus or cell-derived nanovesicle comprising a glycoprotein from Kumasi virus (KRV-G), or a functional fragment or derivative thereof.

156. The recombinant pseudotyped virus or cell-derived nanovesicle of claim 155, wherein said KRV-G comprises the sequence SEQ ID NO: 15.

157. The recombinant pseudotyped virus or cell-derived nanovesicle of claim 156, wherein said KRV-G consists of the sequence SEQ ID NO: 15.

158. A recombinant pseudotyped virus or cell-derived nanovesicle comprising a glycoprotein from Keuraliba virus (KEUV-G), or a functional fragment or derivative thereof.

159. The recombinant pseudotyped virus or cell-derived nanovesicle of claim 158, wherein said KEUV-G comprises the sequence SEQ ID NO: 17.

160. The recombinant pseudotyped virus or cell-derived nanovesicle of claim 159, wherein said KEUV-G consists of the sequence SEQ ID NO: 17.

161. The recombinant pseudotyped virus or cell-derived nanovesicle of any one of claims 137-160, wherein the cytoplasmic tail of said glycoprotein has been removed or truncated, and optionally replaced with another sequence.

162. The recombinant fusogenic protein of claim 161, wherein the cytoplasmic tail of the glycoprotein is truncated by up to 40 amino acids from the C-terminus.

163. The recombinant pseudotyped virus or cell-derived nanovesicle of claim 162, wherein the cytoplasmic tail of the glycoprotein is truncated by 10 to 40 amino acids from the C-terminus.

164. The recombinant pseudotyped virus or cell-derived nanovesicle of claim 163, wherein the cytoplasmic tail of the glycoprotein is truncated by 30 amino acids from the C-terminus.

165. The recombinant pseudotyped virus or cell-derived nanovesicle of any one of claims 161-164, wherein said glycoprotein further comprises a cytoplasmic tail from VSV-G, or a functional fragment or derivative thereof.

166. The recombinant pseudotyped virus or cell-derived nanovesicle of claim 165, wherein the cytoplasmic tail of VSV-G comprises the sequence CIKLKHTKKRQIYTDIEMNRLGK (SEQ ID NO: 16).

167. The recombinant pseudotyped virus of any one of claims 125-166, wherein said virus is a rhabdovirus.

168. The recombinant rhabdovirus of claim 167, wherein said virus is a recombinant vesicular stomatitis virus (VSV).

169. The recombinant pseudotyped virus of any one of claims 125-166, wherein said virus is a retrovirus.

170. The recombinant pseudotyped virus of claim 169, wherein said retrovirus is a lentivirus (LV).

171. The recombinant pseudotyped virus of any one of claims 125-170, wherein said virus is replication-competent.

172. The recombinant pseudotyped virus of any one of claims 125-170, wherein said virus is non-replicative.

173. The recombinant pseudotyped virus or cell-derived nanovesicle of any one of claims 125-172, wherein said virus further comprises a molecular cargo.

174. The recombinant pseudotyped virus or cell-derived nanovesicle of claim 173, wherein said molecular cargo is a transgene encoding a therapeutic protein, a suicide gene, a toxic protein or peptide, an antibody or a fragment thereof, a chimeric antigen receptor (CAR), a T cell receptor (TCR), a gene editing system or a component(s) thereof, an antisense oligonucleotide, a ribozyme, or an RNAi molecule.

175. The recombinant pseudotyped virus or cell-derived nanovesicle of claim 173, wherein said molecular cargo is a therapeutic protein, a toxic protein or peptide, an antibody or a fragment thereof, a chimeric antigen receptor (CAR), a T cell receptor (TCR), a gene editing system or a component(s) thereof, an antisense oligonucleotide, a ribozyme, or an RNAi molecule.

176. The recombinant pseudotyped virus or cell-derived nanovesicle of claim 173, wherein said molecular cargo is a gene editing ribonucleoprotein complex or a component(s) thereof.

177. The recombinant pseudotyped virus or cell-derived nanovesicle of claim 176, wherein said molecular cargo is Cas9 protein complexed with a guide RNA (gRNA) specific to a gene of interest.

178. A composition comprising the recombinant pseudotyped virus or cell-derived nanovesicle of any one of claims 125-177, and a carrier and/or excipient.

179. A method of decreasing susceptibility to serum neutralization of a recombinant virus or nanovesicle in a subject in need thereof, comprising administering to the subject the recombinant pseudotyped virus or cell-derived nanovesicle of any one of claims 125-177 or the composition of claim 178.

180. A method of enhancing resistance to low-density lipoprotein (LDL)- and/or very-low-density lipoprotein (VLDL)-mediated neutralization in a subject in need thereof, comprising administering to the subject the recombinant pseudotyped virus or cell-derived nanovesicle of any one of claims 125-177 or the composition of claim 178.

181. A method of treating a cancer in a subject in need thereof, comprising administering to the subject a therapeutically effective amount of the recombinant pseudotyped virus or cell-derived nanovesicle of any one of claims 125-177 or the composition of claim 178.

182. The method of claim 181, wherein said method does not include pre-treatment with LDL/VLDL-lowering medications.

183. The method of claim 181, wherein said method further comprises pre-treatment with LDL/VLDL-lowering medications.

184. A method of inducing an immune response in a subject in need thereof, comprising administering to the subject an effective amount of the recombinant pseudotyped virus or cell-derived nanovesicle of any one of claims 125-177 or the composition of claim 178.

185. A method for delivering a molecular cargo to a cell within a subject in need thereof, comprising administering to the subject an effective amount of the recombinant pseudotyped virus or cell-derived nanovesicle of any one of claims 125-177, or a composition comprising said pseudotyped virus or cell-derived nanovesicle and a carrier and/or excipient, wherein the recombinant fusogenic protein within said recombinant pseudotyped virus or cell-derived nanovesicle comprises a targeting molecule which targets said cell.

186. The method of any one of claims 179-185, wherein the subject is human.

187. A method for delivering a molecular cargo to a cell ex vivo, comprising administering to said cell an effective amount of the recombinant pseudotyped virus or cell-derived nanovesicle of any one of claims 125-177, or a composition comprising said pseudotyped virus or cell-derived nanovesicle and a carrier and/or excipient, wherein the recombinant fusogenic protein within said recombinant pseudotyped virus or cell-derived nanovesicle comprises a targeting molecule which targets said cell.


SEQUENCE LISTING (236 sequences)

SEQ ID NO: 1 - protein, 20 aa, synthetic construct
AAASGGSGGGGSGGGGSGPK

SEQ ID NO: 2 - protein, 19 aa, synthetic construct
KRAAASGGSGGGGSGGGGS

SEQ ID NO: 3 - protein, 15 aa, synthetic construct
EAAAKEAAAKEAAAK

SEQ ID NO: 4 - protein, 17 aa, synthetic construct
KREAAAKEAAAKEAAAK

SEQ ID NO: 5 - protein, 23 aa, synthetic construct
AAARGSPKGGGGSGGGGSGGGGS

SEQ ID NO: 6 - protein, 18 aa, synthetic construct
GGGGSGGGGSGGGGSGPK

SEQ ID NO: 7 - protein, 19 aa, synthetic construct
AAAGGGGSGGGGSGGGGSK

SEQ ID NO: 8 - protein, 495 aa, synthetic construct
KFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 9 - protein, 596 aa, Hapavirus flanders
WTHDSGRSFVRQYHDPSWFDQTMVYPIECNSTWQEVNTLNLRCPKSLKIDPKNKLNFELGTVYHPLPSSRYVVNGYICHKQTWISKCEETWYFSTTETNKIENVPITPEDCREAVTIYEMGEYVNPFFPPFYCSWCSTQIDKKTFVIVEPHIVKEDIYNKTFIDPLFLNGYCDQLPCKTIHPDVLWVPQELQKRKDLCNKGTWETGKVFGVLEEKLYQNGYSKDNRFGIDEQWIRSSIYGLRSLVGSCYRGVCQQFGIRFKTGEWWGLEGKDVTVWIKRIIPKCQESQYVSFHHDNSDENIAEAQLVARELVCEEFLGRAKGGDLISPFDLNYLLPLNPGLGPSYRAFKRILKRDSHGGSSPQFRLEKRDCIYSIVHNVTEKVNITNNKLAIGQLFDGSYVYINESEFSRPDYLNNSDNASRDGWFLLSLNGMIKYGNSVYLPHGVSTGLSGIQDIVERGTLMLLDHPKSIAISNQMDLAKNIYTSYFQMNTTSIGSKIENMIIRAKNAVSSYFSQLTNIAWWIGTGILGLLGFIVIKRFHLIQLICGKKHRNGKIKKKNNKLNNGDQEAHVYDTISNTPKTPPHGKGTGVKYFDY

SEQ ID NO: 10 - protein, 509 aa, Vesiculovirus chandipura
SLSIAFPENTKLDWKPVTKNTRYCPMGGEWFLEPGLQEESFLSSTPIGATPSKSDGFLCHAAKWVTTCDFRWYGPKYITHSIHNIKPTRSDCDTALASYKSGTLVSPGFPPESCGYASVTDSEFLVIMITPHHVGVDDYRGHWVDPLFVGGECDQSYCDTIHNSSVWIPADQTKKNICGQSFTPLTVTVAYDKTKEIAAGAIVFKSKYHSHMEGARTCRLSYCGRNGIKFPNGEWVSLDVKTKIQEKPLLPLFKECPAGTEVRSTLQSDGAQVLTSEIQRILDYSLCQNTWDKVERKEPLSPLDLSYLASKSPGKGLAYTVINGTLSFAHTRYVRMWIDGPVLKEMKGKRESPSGISSDIWTQWFKYGDMEIGPNGLLKTAGGYKFPWHLIGMGIVDNELHELSEANPLDHPQLPHAQSIADDSEEIFFGDTGVSKNPVELVTGWFTSWKESLAAGVVLILVVVLIYGVLRCFPVLCTTCRKPKWKKGVERSDSFEMRIFKPNNMRARV

SEQ ID NO: 11 - protein, 505 aa, Vesiculovirus perinet
SFQIVFPEFNNAAWLPYLKTSRYCPQSAEMEFERRVSTTLLSADVPIGVTPTKSDGYLCHAAKWVTTCDFRWYGPKYVTHSIHDLTPAQVDCHEALARYKAGTLFNPGFPPASCGYATITDSEQKVVMITPHHVGIDDYRGKWIDPIFPGGECTTNYCETLHNSSVWLPADEKIVDICAQTFRKIKVTATYPSEGAVTKETISLHSAYHPHVPGTGICRMTYCSKEGLRLPNGEWLGIFYDNRIKTTDVRTVFPACPDGLEVKSTLNSDGANTIAWETQRMLDYALCQSTWDKVQNKEPLSAVDLSYLSARSPGKGLAYTVINGTLHFAHVRYVRTWIDGPVLKDLKGSRFDPTAAQKTLWDQWFPFGSNEIGPNGLLKTPKDFKFPLYIIGTGLVDEDLQELSEAGPIDHPQIPDASGILPNSEQVYYGDTGVSKNPIELIEGWFANWKETVMSIVGLVLLITIVFTVLKCIGTCRSLRRKRKIEKDIELQEIGPYQPTTYRPR

SEQ ID NO: 12 - protein, 511 aa, Vesiculovirus piry
KFQIVFPDQNELEWRPVVGDSRHCPQSSEMQFDGSRSQTILTGKAPVGITPSKSDGFICHAAKWVTTCDFRWYGPKYITHSIHHLRPTTSDCETALQRYKDGSLINLGFPPESCGYATVTDSEAMLVQVTPHHVGVDDYRGHWIDPLFPGGECSTNFCDTVHNSSVWIPKSQKTDICAQSFKNIKMTASYPSEGALVSDRFAFHSAYHPNMPGSTVCIMDFCEQKGLRFTNGEWMGLNVEQSIREKKISAIFPNCVAGTEIRATLESEGARTLTWETQRMLDYSLCQNTWDKVSRKEPLSPLDLSYLSPRAPGKGMAYTVINGTLHSAHAKYIRTWIDYGEMKEIKGGRGEYSKAPELLWSQWFDFGPFKIGPNGLLHTGKTFKFPLYLIGAGIIDEDLHELDEAAPIDHPQMPDAKSVLPEDEEIFFGDTGVSKNPIELIQGWFSNWRESVMAIVGIVLLIVVTFLAIKTVRVLNCLWRPRKKRIVRQEVDVESRLNHFEMRGFPEYVKR

SEQ ID NO: 13 - protein, 527 aa, unidentified
QQDYSYGGKTKDIIGLLPQDKKLHWKTTKLDSIKCPEMGLISNKEEHVVEKWLIERPRTTGKLRNKGKLCHLAKWITKCEYTWYFSKTVSRTIQNLEAHEDDCKQAIREYNQGKLIPGSFPPESCYWASTNEESVIAHIITPHEVTYDPYEDRYLDPLFVHGFCRTSFCETVYESTVWLTDSPGRQSSCKLEGDEPVEVLESYRHNKAGESKFGFWMRGSHIHHMPLSRLCKKEYCGKLGYVNQQGVWFHVTSVQWSYNESISFRHVVENCTESPDLVVLSEEFNDDDLAATLEEMMWDINCLNAVENIQKHKRASLHDLYQISQRHPGPGTAYRLKDGHLESAQANFVALYAPDEHEQNRECLGTVLDHTGDQCHAWDDWTHIANSTYHAVNGITEVDGKIVFPEYRVLKRRWDLEYSLKHDLRQINHPVISDFVGKVHENIVHKEIKSHSVNAGDLIGNWVTVAESKIGEFFKGFSHSFVTISVFLMVVLTIWIIIRCCQMCKREKPTKIISAKDDIPMVTSSFG

SEQ ID NO: 14 - protein, 641 aa, unidentified
ILWDFRINRLVRNKRKTQKTKLDQIGNGVVIQPIDPKEVHYNIVKPSIQIKNQSHPVIEIDDQKEPELKLEDFDWVSGNEFEGVVNMPVNCLTNWKVINPYAIRCPTFYEHDRYGGGRTVIGTAVHPEEIEHNIIPGFMCQKQTWVTECTEAWYWSTTVKNYVESSPVVEMECLMALAKEKVGTYVDPFFPPAECAWNANSRSSKEFVTLHPHDVRFDFYQYSKVDPLFVGGKCNEKSCPTIHQHVIWIGKNPVPLEGTCNLDRWRQSDIFALETHTSEKKVSDKVTIYLEFIESATYGMRSTKNACWTRFCDVPGIRFNDGEWWGIKSGHNVALDFLPECGKKSLITLHHAVNQDSEFKNRLSLKHYKCTEVLTKLISGSVITPMDISYLISDRPGLQSYYRFSAKQKGNGPVMGGGNNYMIEQKECMYQFVLLESDRFNITKQSDQINVGMTLQGDHIYINLTDFQHTKGNKSNDINRFTMTVNGYLKTGNVLVLPVDEITSESVDNTLYTPIGYHLIEEEEIGNYTTIGDAIEKILILDPRLNRTDIVEETVHFVNNIGKTVSSFFQGTSSLLWWGVTSVFFLVIVLLMRKCGVIDWLLKKKKPKSVERMNKYNSESNRMVDNKTSHGNVNGGFFGNV

SEQ ID NO: 15 - protein, 531 aa, unidentified
DGTPAPVVKNETGDFLWGLGTELILPVEVISKWVEINPEDMRCPSDPVTDHVIGAIVGHTRFSRYSGFHKATQKGFLCHKMRWITACTTTWYFSHDVQRRVEAQEPGTQECLDQVKKRGEGTEEEGSYPPSQCAWNSVNEEAEVVIHLTVHEVRVDPYTMELLDPVFPSGRCNTTACDTVHQSVLWIEDPSKTRPSCGDRVVDEGNIIRSGWTDNVPGAYYLISQHLPPTKIDGACRLSFCGESGLLFENGIWTHDIKLETVVANQTFQNCQADRRAGAVGPTYEIDKLRFDWEAAKEKIKCLDMIEIISATGALSFRRLHHFNPRTPGIHPVYRIVNKTLQMAKAHYVVTNNPMSHKGTRDCLGTYMDHDTRKCVAWHHWVDVGNGTEQGPNGLLVTGDKVSYPHWFIREKTWDPALHIINYLQNADHPIVSHLGRYIDNASRDSLRKDRSENVGDAASAAVTKLAGSIGGVFKDVWHVITTCVTVGVIIIIILIFRRMIGVFWRGREKHPLPKTPNNIYQETHELKSFG

SEQ ID NO: 16 - protein, 23 aa, Vesiculovirus indiana
CIKLKHTKKRQIYTDIEMNRLGK

SEQ ID NO: 17 - protein, 554 aa, Ledantevirus keuraliba
YPIEDQMTIPLKPGIHRTLDGDLDYNDDEHYHSPPLVLPVPNNRSWKPVNLSTLKCPESSHLGPDTHRTLEKWLIFRPKSSILTKIEGVLCHKSRWLTRCQYTWYFSKTISRKIEPIPPTFQECQEAIKLKEEGILENLGFPPPNCYWARTNDEENILIEISEHPMTYDPYLDGVIDSILVGGKCSQKECETVHDSTIWIETQRDTRPSQCDMGTEEQLELVSGLKQIDGNKQKYQHSVFVVGTNYPFMDAKGACKLRFCGKSGMLLSNGLWFNIAHTILPKPEANSNFWSALPDCSSDKQVGVLGEEYEIEKLQATMEDIMWDLDCFRTVDSLAHHKKVSMLDLFRLAKLTPGPGPAYKLIEGTLMMKEVQYVKARRDTKEQANPLCAAYITESTSNQERCIDYSNYDQNGTYKGQVMNGILVTDGVLIFPHERFHLRQWDPEFIIKHDLQQVHHPVIGNFSKKLHDSIHNSLIKDHSANLGDVMGNWVKVAASKVSGFFKEIEKFLIGGLLLVVILLMVGLLCKCKCRRKPKAKNLKANSSGDEMSPNESIF

SEQ ID NO: 18 - protein, 25 aa, synthetic construct
KRAAARGSPKGGGGSGGGGSGGGGS

SEQ ID NO: 19 - protein, 24 aa, synthetic construct
AAARGSPKGGGGSGGGGSGGGGSK

SEQ ID NO: 20 - protein, 16 aa, synthetic construct
KGGGGSGGGGSGGGGS

SEQ ID NO: 21 - protein, 17 aa, synthetic construct
KRGGGGSGGGGSGGGGS

SEQ ID NO: 22 - protein, 26 aa, synthetic construct
AAARGSPKGGGGSGGGGSGGGGSKFT

SEQ ID NO: 23 - protein, 20 aa, synthetic construct
GGGGSGGGGSGGGGSGPKFT

SEQ ID NO: 24 - protein, 21 aa, synthetic construct
AAAGGGGSGGGGSGGGGSKFT

SEQ ID NO: 25 - protein, 22 aa, synthetic construct
AAASGGSGGGGSGGGGSGPKFT

SEQ ID NO: 26 - protein, 19 aa, synthetic construct
AAASGGSGGGGSGGGGSFT

SEQ ID NO: 27 - protein, 17 aa, synthetic construct
GGGGSGGGGSGGGGSFT

SEQ ID NO: 28 - protein, 8 aa, synthetic construct
GGGGGGFT

SEQ ID NO: 29 - protein, 7 aa, synthetic construct
PAPAPFT

SEQ ID NO: 30 - protein, 17 aa, synthetic construct
EAAAKEAAAKEAAAKFT

SEQ ID NO: 31 - protein, 12 aa, synthetic construct
VPGVGVPGVGFT

SEQ ID NO: 32 - protein, 14 aa, synthetic construct
ESKYGPPCPPCPFT

SEQ ID NO: 33 - protein, 17 aa, synthetic construct
GGGGSGGGGSGGGGSGP

SEQ ID NO: 34 - protein, 18 aa, synthetic construct
AAAGGGGSGGGGSGGGGS

SEQ ID NO: 35 - protein, 19 aa, synthetic construct
AAASGGSGGGGSGGGGSGP

SEQ ID NO: 36 - protein, 17 aa, synthetic construct
AAASGGSGGGGSGGGGS

SEQ ID NO: 37 - protein, 15 aa, synthetic construct
GGGGSGGGGSGGGGS

SEQ ID NO: 38 - protein, 6 aa, synthetic construct
GGGGGG

SEQ ID NO: 39 - protein, 5 aa, synthetic construct
PAPAP

SEQ ID NO: 40 - protein, 10 aa, synthetic construct
VPGVGVPGVG

SEQ ID NO: 41 - protein, 12 aa, synthetic construct
ESKYGPPCPPCP

SEQ ID NO: 42 - protein, 40 aa, synthetic construct
  VARIANT 1..40: note = This sequence may encompass 1-10 GGGS repeating units
GGGSGGGSGGGSGGGSGGGSGGGSGGGSGGGSGGGSGGGS

SEQ ID NO: 43 - protein, 50 aa, synthetic construct
  VARIANT 1..50: note = This sequence may encompass 1-10 GGGGS repeating units
GGGGSGGGGSGGGGSGGGGSGGGGSGGGGSGGGGSGGGGSGGGGSGGGGS

SEQ ID NO: 44 - protein, 5 aa, synthetic construct
GSGGS

SEQ ID NO: 45 - protein, 4 aa, synthetic construct
GGGS

SEQ ID NO: 46 - protein, 4 aa, synthetic construct
GGSG

SEQ ID NO: 47 - protein, 5 aa, synthetic construct
GGSGG

SEQ ID NO: 48 - protein, 5 aa, synthetic construct
GSGSG

SEQ ID NO: 49 - protein, 5 aa, synthetic construct
GSGGG

SEQ ID NO: 50 - protein, 5 aa, synthetic construct
GGGSG

SEQ ID NO: 51 - protein, 5 aa, synthetic construct
GSSSG

SEQ ID NO: 52 - protein, 15 aa, synthetic construct
GCGASGGGGSGGGGS

SEQ ID NO: 53 - protein, 10 aa, synthetic construct
GGGGSGGGGS

SEQ ID NO: 54 - protein, 15 aa, synthetic construct
GGGASGGGGSGGGGS

SEQ ID NO: 55 - protein, 15 aa, synthetic construct
GGGGSGGGGSGGGGS

SEQ ID NO: 56 - protein, 10 aa, synthetic construct
GGGASGGGGS

SEQ ID NO: 57 - protein, 20 aa, synthetic construct
GGGGSGGGGSGGGGSGGGGS

SEQ ID NO: 58 - protein, 4 aa, synthetic construct
EIKR

SEQ ID NO: 59 - protein, 4 aa, synthetic construct
LGAK

SEQ ID NO: 60 - protein, 503 aa, unidentified
SIQIVFPSETKLVWKPVLKGTRYCPQSAELNLEPDLKTMAFDSKVPIGITPSNSDGYLCHAAKWVTTCDFRWYGPKYITHSVHSLRPTVSDCKAAVEAYNAGTLMYPGFPPESCGYASITDSEFYVMLVTPHPVGVDDYRGHWVDPLFPTSECNSNFCETVHNATMWIPKDLKTHDVCSQDFQTIRVSVMYPQTKPTKGADLTLKSKFHAHMKGDRVCKMKFCNKNGLRLGNGEWIEVGDEVMLDNSKLLSLFPDCLVGSVVKSTLLSEGVQTALWETDRLLDYSLCQNTWEKIDRKEPLSAVDLSYLAPRSPGKGMAYIVANGSLMSAPARYIRVWIDSPILKEIKGKKESASGIDTVLWEQWLPFNGMELGPNGLIKTKSGYKFPLYLLGMGIVDQDLQELSSVNPVDHPHVPIAQAFVSEGEEVFFGDTGVSKNPIELISGWFSDWKETAAALGFAAISVILIIGLMRLLPLLCRRRKQKKVIYKDVELNSFDPRQAFHR

SEQ ID NO: 61 - protein, 504 aa, Vesiculovirus jurona
AIPIFFPSEPQLEWKPVLPGSRYCPQSNEMSLDPDLKKSTISVKVPIGVTPSKSDGYLCHGAKWVSTCDFRWYGPKYITHSIHNLRPTTNDCEDAIKKYEAGTLINPGFPPDSCAYATVTDSEHLVILITPHHVGVDDYRGAWVDDSFPSGVCETNQCDTTHNSSIWIPKTKTRHNICSQTFANLSVTISYREGGAMKGADMVFHSKYHPHMVGGHICKMNFCNKQGLRLQNEEWIEIPSGTKVGNQDLMNLFSDCKSGLEVRSTLRSEGANTLTWETQRLLDYALCQNTWDKFDNQGAVSALDLSYLAARAPGKGVAYTMINGTLHSAPTRYVRMWIESPSMEELKAKKESSSGVETSIWNQWFPFKGGEIGPNGLIKAGNKYKFPLYLVGMGMLDDEINALELGGPIDHPQRAHAQAVLGDEETLFFGDTGVGKNPVELITGWFSGWKETIMAVVAIFLLVIVLYGVLRCCPTICVLCKRKSRHRTKDMEMQYIPNNQRHWR

SEQ ID NO: 62 - protein, 499 aa, Rhinolophus euryale
VPVIVPAGRDGRWNDVPSGYRYCPAVGDEFSYRRSMSSPVNLYFAQSPEHTGHEGYLCHVAVYESTCDFRWYGVKYRNQRISRRIPTDPECVSRLGQLKEGRSDWVGFPSFSCNYAAVTTEKRSEIILIPHHVGVDDYKGFFVDPTLRDGLCVKSPCKTVYEDTLWLPSEDLDRGGPCDLKFSETKGSINYPPITRGLTLSDLQLVSPLFPTMNLGESCWMQVCGKMGIRTRSGVWVGIKEEPSIAQMKLYETFIHPCPPNLTISAGHFNPGALKMAWDAGRLLGYSLCQKTWDKLERGDQITPLDLSYLNPSSPGPGLGFMSINGTLKMAKIRFKRMELDGGTINNYDRDNANANVFQWQRWVPHGNVLLGPNGITLNGSTVKFPFYMIGMGRLDSDLIESELIDMVNHVEIKHTHILTPIHDRYGWRPSGEAGDLVRDVRNFFHIPDWIRYVLISVVAVIGLTVVGAVVLKVVRKPRTLRNPESPSHIPLNSMSSFS

SEQ ID NO: 63 - protein, 502 aa, Vesiculovirus malpais
DLPIVFPDQKELLWNPVLKTNRYCPQTREIAPLDKPKTLKITTGVPVRSPKEKIEGYLCHSGKWVTTCDYRWYGAKYVTHSIHHLKPTDQMCRDAISQYNGGTLLNPGFPPEVCGYASVTDSELIITLITPHTVGVDDYRGLWIDPSFPNGECNSIVCETIHNSTKWVSKGEMPTDICQQTFTTIKMDVSYPSDTTSQGSLLSFHSPYHPHISGKDICKMSYCGSNGLRLPNGEWFSIINTSKIGNKNLIDFFSPCKAGVEVRSTLQSEGSQTIAWETQRMLDYALCQNTWDKFERGEPLSPLDLNYLAPRVPGKGMAYTIINNTLHSSHAVYRRVWIEGPIIGEMKGKIESATGVAKEIWAQWFEFGQNKIGPNGVIKTNDGIKFPLYAIGTGLIDQDIHELSEVSPMDHPHLVHAKKYVSEDDEIYFGDTGVSHNPVEIFSGWFTNWKEGLMKFSILVLSILIFYVVIRLVMCIPLKCKKERKPRLEFELQPREWEYSRA

SEQ ID NO: 64 - protein, 538 aa, Vesiculovirus radi
EMMIPFPDVTTTTWKPVLKGEHHCPSSSDVDILSRMSTLKLQVRIPTGSVASKSDGLLCHGAKWVTTCDFRWYGSKYITHSLHSIRPTLSQCTEAAKAYKEGRLMAPGFPPESCGWNSVTDSELLSILVTPHHTGVDDYRGIWIDSMFPGGECKEMVCDTVQGHTIWMSTSNLTTACGVAFKQIQGQFYYLNSGHQPNKEGTFFHSPNHPNSPLSTACRKKYCNQEGIVIHTGEWIGVPWNTRIRDVQLDSYTDLCAESTEIKSTIGSAPIRVIAWEMERVMDFALCQTVWDKVNRGDPLSPLDLSYLSSRAPGKGLAYTIINETLHVAHVRYIRTYIKAPIMEEIKGSRGDRSAAESVLWTQWFPYGDGEIGPNGLLKTNGSFKFPFYLVGMGAIDDDLIELSNADPIDHPQKAIASVHLNTDEELFFGNTGSDSNPVEAVEGWFASWKSAGINMALIVLCVLLVLIFLRSLPALIKLIHRYRVSRSRQTDVELNSINETARTGSVGPDIIPGAWRVHDSGVRQSQFFRNNPRRLGP

SEQ ID NO: 65 - protein, 503 aa, Rhinolophus affinis
VPVILPAGRDGKWNDVPNGFRYCPAVGDEYSYGRSLSLQITLLQPKSKEHEGAEGYLCHVAVYEATCDFRWYGVKYRTQKIRRIAPSEHDCIQRVKNSVTGRSDWVGFPAFSCNYAAVTTEKHSEVVLVPHHVGIDDYQGMFVDPTLKDGSCVQPPCQTLYEDTLWLPVNDLQKTIPCDLEFHQSRGVINFPAPRVGMSLANIQVSGPTLPTTSLDGACHMGICGKWGIRLRSGVWIGFKERPVLQGMDLYETFMHGCRANTTISAGHFNPGALKMIWDAGRLLGYSLCQKTWDKLDRGDSITPLDLSYLNPVSPGPGLGFMSINGTLKMAKLRFARQELPEGVIVNYNRDEPNTLIFQWQRWVPHGNVLVGPNGITLNGTTVKFPFFMVGVGKLDSDLTEAESIDLLHHHEIAKSHMLHPIDDRYEWGQGGKSGDLVREIESWFMIPNWVKQLAYGLTAILVFLAVCFLLNRYCLKSLRCRRKPRSEPPHNRSDIPMSASFL

SEQ ID NO: 66 - protein, 542 aa, unidentified
DMIIPFPDVTTTSWKPVLRGEHHCPASNDLDMAGGLSTLKMNVKIPSGVVGSKSDGYLCHGAKWVTTCDYRWYGAKYITHSLHPLRPSTSQCFDAIKAYREGTLLSPGFPPESCGWNSVTDSELLSIQITPHHSGVDDYRGVWIDSMFPKGECDQRICDTVQEHSIWIAANNVSSACSIAFKQLEGYFYYRNSGIQPNKDGTFFHSSHHPNSPMSSCCRIKYCNQEGLRLHTGEWIGVAWNTKIRDVTLDSYTDTCPGGTEVKSTIGSSPTRVVAWEMERIMDFALCQNVWDKVNRGEQLSPLDLSYLSSRAPGKGLAYTIINETLHVAHVRYIRTWIKGPVLKEIKGRRGSSSAAEDTLWIQWFPFGDNQIGPNGLLKSNGTFKFPFYLVGVGALDEDLIEMANADPVDHLQRVDAETHMRGDEELFFGDTGVSKNPIESVEGWFSNWISGLFNISIIVLCVLSVLIVFKSVITLIRVVRRRRRPRAEEDVELNNMNPRPQTRQPVGAPNIIPGAWGIQPSHGRGVRQSQFVKRSALNIVT

SEQ ID NO: 67 - protein, 497 aa, unidentified
VPVILPAGRDGKWNDVPNGFRYCPAVGDEYSYGRAQTTAITILQPKSLEHEGAEGYLCHVAVYEATCDFRWYGVKYKTQKIRRQIPTDAECQGKQEALMAGRSDWVGFPSFSCNYASVTTEKHLEIVLVPHHVGVDDYLGLYVDPTLQDGSCVTPPCHTLYEDTLWLPKDSPKPGGPCDLEFHEHHGTIRYPIPRAGMSMANFQISGPTIPTATLDGACHMGICGKWGIRLRSGVWVGVKERPMLQGMDLYETFMHPCAPNATISAGHFNPGALKMIWDAGRLLGYSLCQKTWDKLDRGDSITPLDLSYLNPVSPGPGLGFLSINGTLKMAKLRFARVELPEGMIRNYDSNSLNPYQFQWQRWVPHGNVLVGPNGITLNGSTVKFPFFMVGVGRLDSDLTEAESIDLLTHHDIKHSHMLHPVDDRYDWSKSGESGDLIKHIESWFTIPAWIKYCVVVGVCILLIIVFGCMILVKLRKRPSPPVRRDPEIPLSSTSFL

SEQ ID NO: 68 - protein, 633 aa, unidentified
QRVYNFPFNCTEPERIKDYQIKCPIRQNEVSLEAHHVEVDEKIEKICRPQIKDDDHIEGYICREQHWTTKCTETWYFSTEIEYTIKETVPNQADCQKELEKLKRGISIPPYYPPAGCFWNMAQSEKITFVVLVPHKVLQNPYDMKLYDPGFIEKCDVKKAKTKGCKMKDITGLWVTNNDGKNTSEHCNKDHWECIGIKSFRSELNLHDRLWESPELGIMKLNKACKKNFCGYKGVILEDGEWWGYTNVADSEIEYVHLNNCDDSRLPGFRIHQDRTEFEEFDIKAEMENERCMNTLSKILNKENLNFVDMSYLSPSRPGRDYAYLFEQVSWDETFCLTWPDSRKSKNCKVDWKVHKNAGLVTKKHGAIGTYYRSMCMYYPIEDTNKDGILQKDELKDKGIPGKNRYRTLKRSKNDYGEDSEFNITYNGMVVVNESFHMAVKSIYDGTEDYNSLLKFEVSEFDKIDLNEAYKEEENKWNDIDLTPVSSVNRSRSDIIKEVEKGGRKIISAVTGWFTGLAKTVRWTIWGIGSIVTIYAIWKLKKMITKKNKEDKNLVNHNELNEAFEMSKDVERGRVETWIRKNKGKEEGIYEQVSDIEDNVSKYERGVHASKGGDKMNVYSPHGKNGKKGFFNH

SEQ ID NO: 69 - protein, 519 aa, unidentified
VTKPSQYRNLLLPIKVHSDWKPVEIHNLTCPKPRLDWTIDFHQLATEQVYRLKDLSLGEVKGYLCHKAQWITRCEYRWYLSKVVSRRVKEETPTQVECEEAIHQFDEGREMSGSFPPEACFWNAINDESETKIILTVHDLTYDPYTDMGLDSNLVGGTCKSRFCKTTHSSVMWIKDPKSQTEPCVHMVAEKLTILSSKQGETGKRWVKTTATPAMSLEKACKLRYCGIEGILLSSGLWFGLPDSSREHHDNLGIRDNCPTTSEVGVLQPDYLDNSMEFKLEDLQREILCLQLLDKIKLQRLISMFDLQYLRPHNSGFGHVYQVINQTLMSTHAHYAVTTYPGSNSLTRSCLGQYKDSSMNTVCVNWSSWLPMKDGVYQGFNGIIEKDGVILFPEDQLLESSWSPDMFDYIPLDKIHHPFYFNMSEIIHDDIDERLIHDNSTNPGDAISDWVSVANNKVVHFFKQLGESLFIIILIILGANLTYRCVKLCIRWRLGLKSNKTQFAEEGHAMHMARESVFG

SEQ ID NO: 70 - protein, 593 aa, unidentified
LERGSVGSISLISTEKLNLSTSHHDKGIFQNREKPLGHEVVLPVHCTGEWVEKPAMSLACPKRKINGPDGYYDEYFAKMWHPPTHASPEVKGYLCQKTTWNAKCEETWYFSTSKSTTIDETPINEDDCRAALILYKTGELLEPFFPPFSCYWNNININSKTFVTIHEHPTVLDLYKDTKKDPIFLHGECDGEVCETVHSNVLWVEAPLEERDDFCDPALWESSNVYTEKGGPQIPIVLESDVYGPRYTQGMCWMRICGVWGFRFSSGEWWGFRMVNKDFQGLWYTGKIPSCYGDFEVSFAHEAIAMTQLLENLSYKDHKCVDVLSTLRGNKVINAYELSYLVPEHPGFGPAYRILMVKNKRNVTQPVFILQTKNCRYQRAYLTNLSFTITGEETSESVKVGVWGDNQPVYLNWTEIGVNSTYKPNITGNWHQLMTFNGLMRFDKTLLFPQSVFVDAPNVSLLLDGFQLDLIEHPHQYFGRDEKTPSSLYKFYPHGNSTNVGEVIEGWFKSAKNAIGSLFSGMSSLMWWIISTVLSLITLLVCYRCGLLSLVKRMFTKRTRPSKKGRTSRSSHRMEHIYTEPNNPSSSSNPFFA

SEQ ID NO: 71 - protein, 584 aa, unidentified
WTHDVGRDYIHRPHNPDWFDNVISFPTECYTDWTLVRAHEIKCPSLSSINLDDKLSFKLGTVMHPLPNNKYTVDGYICHKQQWISKCEETWYFSTTETNSIENLPIGATECMEAITVYESGEYNNPFFPPFYCSWCSTQIDQKTFIVIEEHTAQENIYNASYIDPMFVGGKCSSNLCKTIHPDVLWVAKREEIRRDACNRKTWETGDVYGLVEEKKFDNDQRDFGIGEQWIRSSIYGVRRLDGSCYSKICGQFGIRFNTGEWWGLDGQGVKIWLRKILKPCQGGLRISFHHDNHDETMAIAHSVAREVTCEEVTGRILNQGFISAFDLAYLNPLNPGRGNVYRVFKRVIKGRHADEQYEYKMEKKYCMYRTLHNVSDVINQTRGKFLLGYFFDGSPFYLNQSDFEGAGKYGNERNTSRDGWFLLTYAGLTKFQQTLYTNEGVSNSQAALRNLHDPGRLALAEETEIPNVRDQMDLANKVYNSWFKMNTTSVGERITTFINNAKSAVSNYFSQLTNVIWWVGTGAIGLIVVILGRRFYRYRKSSKPPALPKRLDSVETQSTHIYEPVRSPQPVARGNQGHPFFSF

SEQ ID NO: 72 - protein, 605 aa, unidentified
YIYSGSVGGYRPKKVRTYGEVIPYTEIVEHRNLYQNWNGKHRTGHRTVLPTHCHTEWKDVTHGSIKCPHRKVIGTDGIYNTYIGDVWHPHTDSGSEIKGFLCQKTRWVSTCIETWYFSTTKETKIEEVVINPEDCLASITLMDSGEYIEPFFPPHVCSWAATNENAKEFVTVHSHPVVVDIYKNEMIDPIFLSGKCKEKVCHTIHKNVIWIEANDNERSDICVASAWESSHVFADLDIEPDIRAQKVEYIGESIDSEIYGPRSLKGACLIKICGIWGIRFSHGEWWGLKTLSNKISFADGLYNCGSNTSVGFIHNIWTPSGLIGEITYRDHKCLDVVSSLLGHQKINPYELSYLIQDFPGEGPAYRIMKQMTGLNRTKSSFKMQMKTCRYHTAYITNVTFQPIDSTQEVYKLGIWGSGHRIILNSTEIGINPTYTNSGSDWELLLTFNGLMRFGSELVLPHAVFSDHPNTSDLLEDYEINLIGHPKEIFQSDQDELAQIYKFYRRANSTNVVSLASNFLKDIGKSIGNFFGGTKNLIWWIVTLALSTLGTFIAYKLGLFKCLKRMILETDNESGNKRISNVYEEPLQLGERGHKPVKNPFFDHGI

SEQ ID NO: 73 - protein, 607 aa, unidentified
IVINYPTACHTYQEVLYQGLECPEPAISYKLDNNETVAYGQICRPQLASKDILEGYLCYKDTYISSCEETWYFTSQVKQTIVHEHVSDAECIESLAYYKSGIVETPMFLNVDCYWNAINSIKKSYLIIVYHPVPFDPYTNSIKDAVVKNSEDVNSWIRDTHYPFTKWIRDFNGTAEEKCDAQHWECFKVNLYKGWIYSPPHTKNTIGSSTQTGLILESDIYSHTLIRDLCRFQFCGIHGFVFQDQSWWDLQLNVSLSSLISTEHLSGAPDGHCKKVNEIGHAELEPNWEKILSVDDYDIRHQLCLDTLASVLGGGFLTARDLLKFAPMRPGLGPAYFLFNPNKRERAVHVWTAGATTSSILWKSTCKYELIDIPQLNDTGIITYEKLDNIIGKILRNDVGVSFKDLGFTENELTDDDVSQSQLNSSLGIYHRNTSMKGIPWKRHRASTPKLKMGPNGILHDLNAKIIHLPQASSSVFKLPPHLYEGHRVVFFNHITKKKIYEDLSKREGNDPYNVDIGDLIGRHLNRTTIPDQLHDWVSGIKRHIFSVFEQFGSLIKVVVFIIMLVLCIKIINLIYRFYKVRKSNHKKLASRKEKLHLSDPFSVNSK

SEQ ID NO: 74 - protein, 611 aa, Ephemerovirus febris
KIHLEKIYNVPVNCGELHPVKAHEIKCPQRLNELSLQAHHNLAKDEHYNKICRPQLKDDAHLEGFICRKQRWITKCSETWYFSTSIEYQILEVIPEYSGCTDAVKKLDQGALIPPYYPPAGCFWNTEMNQEIEFYVLIQHKPFLNPYDNLIYDSRFLTPCTINDSKTKGCPLKDITGTWIPDVRVEEISEHCNNKHWECITVKSFRSELNDKERLWEAPDIGLVHVNKGCLSTFCGKNGIIFEDGEWWSIENQTESDFQNFKIEKCKGKKPGFRMHTDRTEFEELDIKAELEHERCLNTISKILNKENINTLDMSYLAPTRPGRDYAYLFEQTSWQEKLCLSLPDSGRVSKDCNIDWRTSTRGGMVKKNHYGIGSYKRAWCEYRPFVDKNEDGYIDIQELNGHNMSGNHAILETAPAGGSSGNRLNVTLNGMIFVEPTKLYLHTKSLYEGIEDYQKLIKFEVMEYDNVEENLIRYEEDEKFKPVNLNPHEKSQINRTDIVREIQKGGKKVLSAVVGWFTSTAKAVRWTIWAVGAIVTTYAIYKLYKMVKSNSSHSKHREADLEGLQSTTKENMRVEKNDKNYQDLELGLYEEIRSIKGGSKQTGDDRFFDH

SEQ ID NO: 75 - protein, 794 aa, unidentified
VSIKDSCEAKSAPWIPCEKFDYVKNATGSGIKCWIFCSRSGFYSKTGRFIRCIQGDPEAKYIKSCRRQIEKRGKEKMREGTRGKRKTSEPKEEGVRAKTDFTPDESRRLNNLTKVFRKVEDKDLNDFKKFILEKGLETKIKLANDGKISFRDPDCGENKDYPCHRIHQIIEGVNENIDYINEILSLKKMKEELRLRERESEEGEFPGLLNTTNRRGFLLHYPVELGNWSRLEDPSQIKCPSHHKDMLSNPRRLGKYNLDIIVRRPRIGTFETVVPGYICQGMQWTSTCNEMWYFVTYHDRAVHYITPNKLKCLQNIRAHKRGEHIKPYYPLEECNWNSETTKTVDYFMITPYSPEVDPFTLEFKSEIFPDRTSCRPGDEICVTDDDSKVWFPDEDDKLIARGHCPDETWDESHLTIHPEEMPENWEDPQSPWVSDYILKGVLFGEKRVKKSCLLEFCGTSGLLFEDGEWWELNVFSREKGRESLTKIFIEQEEIRRCNGTETRVGVAGKETDEKALLNAVLSKNAYERCKSARYRLIENKYLRLDDLSYINPRESVTWWAYRVRAGDDERTFKLEKTTGEYRYLQVPPSLEQHVTDCDGQENCSVSIGYYRGELINSSDWTRTGHDDVYVGVNGLLRKDTGNKTIVLYPPLMKEYQEIFSDSGESDDEAFIYKPDIHEKKGKPKEAEDEKDEKSKKNKTPIDDIKDWWSNIKGEWHLIKGILIGLFTFALLIGVVKLGVFIKSSFRKRRDDSIPEGKDEEIGIKMQSRRSRQNIYEEINEVSPTMTRRGRNIFN

SEQ ID NO: 76 - protein, 531 aa, Sigmavirus melanogaster
QKAFTPDLVFPEMNRNSSWSVANYGEILCPTSFQSYDPKKHQILTRVLVERPSLNTDTKVEGYTCHKVKYETICDMPWYFSPTISHSISPLRVKESECKDAIAEHQLGTHVPLSFPPEDCSWNSVNTKEYEDIIVKEHPVMLDPYTNNYVDAIFPGGISSPGMGGTIHDDMMWVSKDLAVSPECSGWQRSMGLIYSSRLYGEREPMLEVGSIHIEGHRDKNLTLACRISFCGEIGVRFHDGEWMKVSVNLDHPNSVTFQVTDFPPCPPGTTIQTAVVENINPEIQELTVNMMYRLKCQETISKMVSGLPTSALDLSYLIQVQEGPGIVYKREKGILYQSVGMYQYIDTVTLNKEENQLGENSRGQKVFWTEWSDSPTRPGLQEGINGIVKYEGQVRVPLGMSLRLEAATELMWGHPVHTVSHPILHVISNHTEQSVTTWNRGVNSTNLIGLATRSISGFYDNLKLYLILALIFVSLIALVVLDVIPFKYILFVLCPPLLLCRFIKCSRRKPETRDRYHVEYNRPGQVSSAF

SEQ ID NO: 77 - protein, 490 aa, unidentified
VFHYPIEKDIHWYPANHSSLRCPIRSASITDTPTGGVTISIPSNPSNNDLPGFSCHKTEWISECTETWYWSTDVKQYIRPVSVTADECKKAQRDKEVGTEITPFFTAPVCQWSNTVRKVNSFVITNKKNVKFDPYNLDFIDPILVGGRCKGNQESCPTIQAGVIWLPRLQPTKATSWTNIYAKYKRVGPHMGDWKFWGGGMPTSTFKDACKMEFRGKEGIRVSSGFWFHIPQMDDVEFKTEYGKLAHCVSSKEIKFPSAHEEVAEHEMEIQDLILTLRCRDIIDKYEETGSISFMDLALFDPDNEGPAHIYRINKGKLEAGLVNYGECKVSKKGDPAESACVKVMDNGQRSPIFFQDWVPTGIKGIQSGFNGLYRENGEIKHAGYNLFQNKLTESDIQRMELTPIHHPVLLSLSDVAPGLNVTFDQTGERGELDLDLLPGITGIWRKFVEYLSMAALILTLIVSIFVVWKCCISNHLGPSKKTSEMEYFE

SEQ ID NO: 78 - protein, 450 aa, unidentified
RDDLTCPIYNHQNVEFVNTTITYIRLNSVLLQSKSLELKQMPYVRGHICQKIRYITTCKANLFTSNEIFYKKEYLTVTKNDCEMKAVHETGEYPAPICTWSLFGSNLHNEEIIVTEIQSHDYHYDLFNGKIKDSEYLFEHCTLMYCKLREHKGYWIKSEPTKNDICPIVQDDEIVAELKFYNENHFLKINHHLYSTEEVCKIKYCNNSLLFFKDIGFFNIKSTLQKMDKIFKKCTKIEDLRYIIHDNVEKIDNLNDCLNFKLNVLTNKDRTIAYHDIRKLHPKGTGINRIYRLNGNNTLESAIAYYGSVESNETKIYLDYWNDCSKTHTCTYNGYMGKKGLNIRARLNIDLFQEVYEEDSSLLIYNGTTDLSTGTIGELTKGDANVTFTTKEWIPHISNYIIIIVFCLLSGAVFIIMINIYNRIMVMKRNRKAFYNRENDNRVIYVNDWK

SEQ ID NO: 79 - protein, 510 aa, unidentified
NRVVAPIHEPQNWKPATVDDFTCRTGFNLDFDSKFIKTKALVLKRVGQAKVKGYLCMKNRWTTTCETNWLYSKSVSHHITHVAVSAEECYNKIRDDASGNLKIESYPNPQCAWSSTVSREEDFIHISTSDVGYDMYTDTVLSPSFPGGTCKLKTCCKTIYPNIVWVPETPAQTQVRDALFDETMVTVTVEAKKVVKDSWVTGATITPSVMEGSCKKTLGSKSGILLPNGQWFSIVETGQITIQPKGSVEEKETWVNLINDLNLSDCAETQEAKVPTAEFTVYKTESMVFNILNYHLCLETVAKARSGKNLTRLDLARLAPEIPGVAHVYQLTSDGVRVGSTRYEIIAWKPTMGLDKTLGLTIVPSGNRNSETIKWIEWTRTDDGLLNGPNGIFIADGKEIVHPNLKMVSFELETYLISEHSTQLVPHPVIHSISDEIYPENYTIGGKNSYIKIHTPTAYFWSGIHWIEGAVQKLFIVVVATALIGLFILVVWLCCGCCSKSRPVRNQKWE

SEQ ID NO: 80 - protein, 511 aa, synthetic construct
MKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 81 - protein, 618 aa, synthetic construct
MSYLIVKVVILFLVGIDKQVLSWTHDSGRSFVRQYHDPSWFDQTMVYPIECNSTWQEVNTLNLRCPKSLKIDPKNKLNFELGTVYHPLPSSRYVVNGYICHKQTWISKCEETWYFSTTETNKIENVPITPEDCREAVTIYEMGEYVNPFFPPFYCSWCSTQIDKKTFVIVEPHIVKEDIYNKTFIDPLFLNGYCDQLPCKTIHPDVLWVPQELQKRKDLCNKGTWETGKVFGVLEEKLYQNGYSKDNRFGIDEQWIRSSIYGLRSLVGSCYRGVCQQFGIRFKTGEWWGLEGKDVTVWIKRIIPKCQESQYVSFHHDNSDENIAEAQLVARELVCEEFLGRAKGGDLISPFDLNYLLPLNPGLGPSYRAFKRILKRDSHGGSSPQFRLEKRDCIYSIVHNVTEKVNITNNKLAIGQLFDGSYVYINESEFSRPDYLNNSDNASRDGWFLLSLNGMIKYGNSVYLPHGVSTGLSGIQDIVERGTLMLLDHPKSIAISNQMDLAKNIYTSYFQMNTTSIGSKIENMIIRAKNAVSSYFSQLTNIAWWIGTGILGLLGFIVIKRFHLIQLICGKKHRNGKIKKKNNKLNNGDQEAHVYDTISNTPKTPPHGKGTGVKYFDY

SEQ ID NO: 82 - protein, 530 aa, synthetic construct
MTSSVTISVILLISFIAPSYSSLSIAFPENTKLDWKPVTKNTRYCPMGGEWFLEPGLQEESFLSSTPIGATPSKSDGFLCHAAKWVTTCDFRWYGPKYITHSIHNIKPTRSDCDTALASYKSGTLVSPGFPPESCGYASVTDSEFLVIMITPHHVGVDDYRGHWVDPLFVGGECDQSYCDTIHNSSVWIPADQTKKNICGQSFTPLTVTVAYDKTKEIAAGAIVFKSKYHSHMEGARTCRLSYCGRNGIKFPNGEWVSLDVKTKIQEKPLLPLFKECPAGTEVRSTLQSDGAQVLTSEIQRILDYSLCQNTWDKVERKEPLSPLDLSYLASKSPGKGLAYTVINGTLSFAHTRYVRMWIDGPVLKEMKGKRESPSGISSDIWTQWFKYGDMEIGPNGLLKTAGGYKFPWHLIGMGIVDNELHELSEANPLDHPQLPHAQSIADDSEEIFFGDTGVSKNPVELVTGWFTSWKESLAAGVVLILVVVLIYGVLRCFPVLCTTCRKPKWKKGVERSDSFEMRIFKPNNMRARV

SEQ ID NO: 83 - protein, 523 aa, synthetic construct
MTSVLFMVGVLLGAFGSTHCSIQIVFPSETKLVWKPVLKGTRYCPQSAELNLEPDLKTMAFDSKVPIGITPSNSDGYLCHAAKWVTTCDFRWYGPKYITHSVHSLRPTVSDCKAAVEAYNAGTLMYPGFPPESCGYASITDSEFYVMLVTPHPVGVDDYRGHWVDPLFPTSECNSNFCETVHNATMWIPKDLKTHDVCSQDFQTIRVSVMYPQTKPTKGADLTLKSKFHAHMKGDRVCKMKFCNKNGLRLGNGEWIEVGDEVMLDNSKLLSLFPDCLVGSVVKSTLLSEGVQTALWETDRLLDYSLCQNTWEKIDRKEPLSAVDLSYLAPRSPGKGMAYIVANGSLMSAPARYIRVWIDSPILKEIKGKKESASGIDTVLWEQWLPFNGMELGPNGLIKTKSGYKFPLYLLGMGIVDQDLQELSSVNPVDHPHVPIAQAFVSEGEEVFFGDTGVSKNPIELISGWFSDWKETAAALGFAAISVILIIGLMRLLPLLCRRRKQKKVIYKDVELNSFDPRQAFHR

SEQ ID NO: 84 - protein, 524 aa, synthetic construct
MESLPFSALLAVLSITLCDSAIPIFFPSEPQLEWKPVLPGSRYCPQSNEMSLDPDLKKSTISVKVPIGVTPSKSDGYLCHGAKWVSTCDFRWYGPKYITHSIHNLRPTTNDCEDAIKKYEAGTLINPGFPPDSCAYATVTDSEHLVILITPHHVGVDDYRGAWVDDSFPSGVCETNQCDTTHNSSIWIPKTKTRHNICSQTFANLSVTISYREGGAMKGADMVFHSKYHPHMVGGHICKMNFCNKQGLRLQNEEWIEIPSGTKVGNQDLMNLFSDCKSGLEVRSTLRSEGANTLTWETQRLLDYALCQNTWDKFDNQGAVSALDLSYLAARAPGKGVAYTMINGTLHSAPTRYVRMWIESPSMEELKAKKESSSGVETSIWNQWFPFKGGEIGPNGLIKAGNKYKFPLYLVGMGMLDDEINALELGGPIDHPQRAHAQAVLGDEETLFFGDTGVGKNPVELITGWFSGWKETIMAVVAIFLLVIVLYGVLRCCPTICVLCKRKSRHRTKDMEMQYIPNNQRHWR

SEQ ID NO: 85 - protein, 514 aa, synthetic construct
MNVWRFLSLVPLATSVPVIVPAGRDGRWNDVPSGYRYCPAVGDEFSYRRSMSSPVNLYFAQSPEHTGHEGYLCHVAVYESTCDFRWYGVKYRNQRISRRIPTDPECVSRLGQLKEGRSDWVGFPSFSCNYAAVTTEKRSEIILIPHHVGVDDYKGFFVDPTLRDGLCVKSPCKTVYEDTLWLPSEDLDRGGPCDLKFSETKGSINYPPITRGLTLSDLQLVSPLFPTMNLGESCWMQVCGKMGIRTRSGVWVGIKEEPSIAQMKLYETFIHPCPPNLTISAGHFNPGALKMAWDAGRLLGYSLCQKTWDKLERGDQITPLDLSYLNPSSPGPGLGFMSINGTLKMAKIRFKRMELDGGTINNYDRDNANANVFQWQRWVPHGNVLLGPNGITLNGSTVKFPFYMIGMGRLDSDLIESELIDMVNHVEIKHTHILTPIHDRYGWRPSGEAGDLVRDVRNFFHIPDWIRYVLISVVAVIGLTVVGAVVLKVVRKPRTLRNPESPSHIPLNSMSSFS

SEQ ID NO: 86 - protein, 521 aa, synthetic construct
MESLLKAICVLLLIHCSRCDLPIVFPDQKELLWNPVLKTNRYCPQTREIAPLDKPKTLKITTGVPVRSPKEKIEGYLCHSGKWVTTCDYRWYGAKYVTHSIHHLKPTDQMCRDAISQYNGGTLLNPGFPPEVCGYASVTDSELIITLITPHTVGVDDYRGLWIDPSFPNGECNSIVCETIHNSTKWVSKGEMPTDICQQTFTTIKMDVSYPSDTTSQGSLLSFHSPYHPHISGKDICKMSYCGSNGLRLPNGEWFSIINTSKIGNKNLIDFFSPCKAGVEVRSTLQSEGSQTIAWETQRMLDYALCQNTWDKFERGEPLSPLDLNYLAPRVPGKGMAYTIINNTLHSSHAVYRRVWIEGPIIGEMKGKIESATGVAKEIWAQWFEFGQNKIGPNGVIKTNDGIKFPLYAIGTGLIDQDIHELSEVSPMDHPHLVHAKKYVSEDDEIYFGDTGVSHNPVEIFSGWFTNWKEGLMKFSILVLSILIFYVVIRLVMCIPLKCKKERKPRLEFELQPREWEYSRA

SEQ ID NO: 87 - protein, 525 aa, synthetic construct
MSSKIVLAAICLCSVQYVACSFQIVFPEFNNAAWLPYLKTSRYCPQSAEMEFERRVSTTLLSADVPIGVTPTKSDGYLCHAAKWVTTCDFRWYGPKYVTHSIHDLTPAQVDCHEALARYKAGTLFNPGFPPASCGYATITDSEQKVVMITPHHVGIDDYRGKWIDPIFPGGECTTNYCETLHNSSVWLPADEKIVDICAQTFRKIKVTATYPSEGAVTKETISLHSAYHPHVPGTGICRMTYCSKEGLRLPNGEWLGIFYDNRIKTTDVRTVFPACPDGLEVKSTLNSDGANTIAWETQRMLDYALCQSTWDKVQNKEPLSAVDLSYLSARSPGKGLAYTVINGTLHFAHVRYVRTWIDGPVLKDLKGSRFDPTAAQKTLWDQWFPFGSNEIGPNGLLKTPKDFKFPLYIIGTGLVDEDLQELSEAGPIDHPQIPDASGILPNSEQVYYGDTGVSKNPIELIEGWFANWKETVMSIVGLVLLITIVFTVLKCIGTCRSLRRKRKIEKDIELQEIGPYQPTTYRPR

SEQ ID NO: 88 - protein, 529 aa, synthetic construct
MDLFPILVVVLMTDTVLGKFQIVFPDQNELEWRPVVGDSRHCPQSSEMQFDGSRSQTILTGKAPVGITPSKSDGFICHAAKWVTTCDFRWYGPKYITHSIHHLRPTTSDCETALQRYKDGSLINLGFPPESCGYATVTDSEAMLVQVTPHHVGVDDYRGHWIDPLFPGGECSTNFCDTVHNSSVWIPKSQKTDICAQSFKNIKMTASYPSEGALVSDRFAFHSAYHPNMPGSTVCIMDFCEQKGLRFTNGEWMGLNVEQSIREKKISAIFPNCVAGTEIRATLESEGARTLTWETQRMLDYSLCQNTWDKVSRKEPLSPLDLSYLSPRAPGKGMAYTVINGTLHSAHAKYIRTWIDYGEMKEIKGGRGEYSKAPELLWSQWFDFGPFKIGPNGLLHTGKTFKFPLYLIGAGIIDEDLHELDEAAPIDHPQMPDAKSVLPEDEEIFFGDTGVSKNPIELIQGWFSNWRESVMAIVGIVLLIVVTFLAIKTVRVLNCLWRPRKKRIVRQEVDVESRLNHFEMRGFPEYVKR

SEQ ID NO: 89 - protein, 556 aa, synthetic construct
MISITFVYLIIILSLSWGEMMIPFPDVTTTTWKPVLKGEHHCPSSSDVDILSRMSTLKLQVRIPTGSVASKSDGLLCHGAKWVTTCDFRWYGSKYITHSLHSIRPTLSQCTEAAKAYKEGRLMAPGFPPESCGWNSVTDSELLSILVTPHHTGVDDYRGIWIDSMFPGGECKEMVCDTVQGHTIWMSTSNLTTACGVAFKQIQGQFYYLNSGHQPNKEGTFFHSPNHPNSPLSTACRKKYCNQEGIVIHTGEWIGVPWNTRIRDVQLDSYTDLCAESTEIKSTIGSAPIRVIAWEMERVMDFALCQTVWDKVNRGDPLSPLDLSYLSSRAPGKGLAYTIINETLHVAHVRYIRTYIKAPIMEEIKGSRGDRSAAESVLWTQWFPYGDGEIGPNGLLKTNGSFKFPFYLVGMGAIDDDLIELSNADPIDHPQKAIASVHLNTDEELFFGNTGSDSNPVEAVEGWFASWKSAGINMALIVLCVLLVLIFLRSLPALIKLIHRYRVSRSRQTDVELNSINETARTGSVGPDIIPGAWRVHDSGVRQSQFFRNNPRRLGP

SEQ ID NO: 90 - protein, 520 aa, synthetic construct
MYAQYITISLFALQAHSVPVILPAGRDGKWNDVPNGFRYCPAVGDEYSYGRSLSLQITLLQPKSKEHEGAEGYLCHVAVYEATCDFRWYGVKYRTQKIRRIAPSEHDCIQRVKNSVTGRSDWVGFPAFSCNYAAVTTEKHSEVVLVPHHVGIDDYQGMFVDPTLKDGSCVQPPCQTLYEDTLWLPVNDLQKTIPCDLEFHQSRGVINFPAPRVGMSLANIQVSGPTLPTTSLDGACHMGICGKWGIRLRSGVWIGFKERPVLQGMDLYETFMHGCRANTTISAGHFNPGALKMIWDAGRLLGYSLCQKTWDKLDRGDSITPLDLSYLNPVSPGPGLGFMSINGTLKMAKLRFARQELPEGVIVNYNRDEPNTLIFQWQRWVPHGNVLVGPNGITLNGTTVKFPFFMVGVGKLDSDLTEAESIDLLHHHEIAKSHMLHPIDDRYEWGQGGKSGDLVREIESWFMIPNWVKQLAYGLTAILVFLAVCFLLNRYCLKSLRCRRKPRSEPPHNRSDIPMSASFL

SEQ ID NO: 91 - protein, 559 aa, synthetic construct
MISSTLILVIISAHAFCDMIIPFPDVTTTSWKPVLRGEHHCPASNDLDMAGGLSTLKMNVKIPSGVVGSKSDGYLCHGAKWVTTCDYRWYGAKYITHSLHPLRPSTSQCFDAIKAYREGTLLSPGFPPESCGWNSVTDSELLSIQITPHHSGVDDYRGVWIDSMFPKGECDQRICDTVQEHSIWIAANNVSSACSIAFKQLEGYFYYRNSGIQPNKDGTFFHSSHHPNSPMSSCCRIKYCNQEGLRLHTGEWIGVAWNTKIRDVTLDSYTDTCPGGTEVKSTIGSSPTRVVAWEMERIMDFALCQNVWDKVNRGEQLSPLDLSYLSSRAPGKGLAYTIINETLHVAHVRYIRTWIKGPVLKEIKGRRGSSSAAEDTLWIQWFPFGDNQIGPNGLLKSNGTFKFPFYLVGVGALDEDLIEMANADPVDHLQRVDAETHMRGDEELFFGDTGVSKNPIESVEGWFSNWISGLFNISIIVLCVLSVLIVFKSVITLIRVVRRRRRPRAEEDVELNNMNPRPQTRQPVGAPNIIPGAWGIQPSHGRGVRQSQFVKRSALNIVT

SEQ ID NO: 92 - protein, 512 aa, synthetic construct
MWSAVISSIIPLVLSVPVILPAGRDGKWNDVPNGFRYCPAVGDEYSYGRAQTTAITILQPKSLEHEGAEGYLCHVAVYEATCDFRWYGVKYKTQKIRRQIPTDAECQGKQEALMAGRSDWVGFPSFSCNYASVTTEKHLEIVLVPHHVGVDDYLGLYVDPTLQDGSCVTPPCHTLYEDTLWLPKDSPKPGGPCDLEFHEHHGTIRYPIPRAGMSMANFQISGPTIPTATLDGACHMGICGKWGIRLRSGVWVGVKERPMLQGMDLYETFMHPCAPNATISAGHFNPGALKMIWDAGRLLGYSLCQKTWDKLDRGDSITPLDLSYLNPVSPGPGLGFLSINGTLKMAKLRFARVELPEGMIRNYDSNSLNPYQFQWQRWVPHGNVLVGPNGITLNGSTVKFPFFMVGVGRLDSDLTEAESIDLLTHHDIKHSHMLHPVDDRYDWSKSGESGDLIKHIESWFTIPAWIKYCVVVGVCILLIIVFGCMILVKLRKRPSPPVRRDPEIPLSSTSFL

SEQ ID NO: 93 - protein, 543 aa, synthetic construct
MDFWMIIYVLVVSASAQQDYSYGGKTKDIIGLLPQDKKLHWKTTKLDSIKCPEMGLISNKEEHVVEKWLIERPRTTGKLRNKGKLCHLAKWITKCEYTWYFSKTVSRTIQNLEAHEDDCKQAIREYNQGKLIPGSFPPESCYWASTNEESVIAHIITPHEVTYDPYEDRYLDPLFVHGFCRTSFCETVYESTVWLTDSPGRQSSCKLEGDEPVEVLESYRHNKAGESKFGFWMRGSHIHHMPLSRLCKKEYCGKLGYVNQQGVWFHVTSVQWSYNESISFRHVVENCTESPDLVVLSEEFNDDDLAATLEEMMWDINCLNAVENIQKHKRASLHDLYQISQRHPGPGTAYRLKDGHLESAQANFVALYAPDEHEQNRECLGTVLDHTGDQCHAWDDWTHIANSTYHAVNGITEVDGKIVFPEYRVLKRRWDLEYSLKHDLRQINHPVISDFVGKVHENIVHKEIKSHSVNAGDLIGNWVTVAESKIGEFFKGFSHSFVTISVFLMVVLTIWIIIRCCQMCKREKPTKIISAKDDIPMVTSSFG

SEQ ID NO: 94 - protein, 665 aa, synthetic construct
MILIRWFLWISLSLLLLGINEVDGILWDFRINRLVRNKRKTQKTKLDQIGNGVVIQPIDPKEVHYNIVKPSIQIKNQSHPVIEIDDQKEPELKLEDFDWVSGNEFEGVVNMPVNCLTNWKVINPYAIRCPTFYEHDRYGGGRTVIGTAVHPEEIEHNIIPGFMCQKQTWVTECTEAWYWSTTVKNYVESSPVVEMECLMALAKEKVGTYVDPFFPPAECAWNANSRSSKEFVTLHPHDVRFDFYQYSKVDPLFVGGKCNEKSCPTIHQHVIWIGKNPVPLEGTCNLDRWRQSDIFALETHTSEKKVSDKVTIYLEFIESATYGMRSTKNACWTRFCDVPGIRFNDGEWWGIKSGHNVALDFLPECGKKSLITLHHAVNQDSEFKNRLSLKHYKCTEVLTKLISGSVITPMDISYLISDRPGLQSYYRFSAKQKGNGPVMGGGNNYMIEQKECMYQFVLLESDRFNITKQSDQINVGMTLQGDHIYINLTDFQHTKGNKSNDINRFTMTVNGYLKTGNVLVLPVDEITSESVDNTLYTPIGYHLIEEEEIGNYTTIGDAIEKILILDPRLNRTDIVEETVHFVNNIGKTVSSFFQGTSSLLWWGVTSVFFLVIVLLMRKCGVIDWLLKKKKPKSVERMNKYNSESNRMVDNKTSHGNVNGGFFGNV

SEQ ID NO: 95 - protein, 573 aa, synthetic construct
MECIMFWTILALSISFGFSYPIEDQMTIPLKPGIHRTLDGDLDYNDDEHYHSPPLVLPVPNNRSWKPVNLSTLKCPESSHLGPDTHRTLEKWLIFRPKSSILTKIEGVLCHKSRWLTRCQYTWYFSKTISRKIEPIPPTFQECQEAIKLKEEGILENLGFPPPNCYWARTNDEENILIEISEHPMTYDPYLDGVIDSILVGGKCSQKECETVHDSTIWIETQRDTRPSQCDMGTEEQLELVSGLKQIDGNKQKYQHSVFVVGTNYPFMDAKGACKLRFCGKSGMLLSNGLWFNIAHTILPKPEANSNFWSALPDCSSDKQVGVLGEEYEIEKLQATMEDIMWDLDCFRTVDSLAHHKKVSMLDLFRLAKLTPGPGPAYKLIEGTLMMKEVQYVKARRDTKEQANPLCAAYITESTSNQERCIDYSNYDQNGTYKGQVMNGILVTDGVLIFPHERFHLRQWDPEFIIKHDLQQVHHPVIGNFSKKLHDSIHNSLIKDHSANLGDVMGNWVKVAASKVSGFFKEIEKFLIGGLLLVVILLMVGLLCKCKCRRKPKAKNLKANSSGDEMSPNESIF

SEQ ID NO: 96 - protein, 655 aa, synthetic construct
MSSEIKMLIELMLFSILSCVISQRVYNFPFNCTEPERIKDYQIKCPIRQNEVSLEAHHVEVDEKIEKICRPQIKDDDHIEGYICREQHWTTKCTETWYFSTEIEYTIKETVPNQADCQKELEKLKRGISIPPYYPPAGCFWNMAQSEKITFVVLVPHKVLQNPYDMKLYDPGFIEKCDVKKAKTKGCKMKDITGLWVTNNDGKNTSEHCNKDHWECIGIKSFRSELNLHDRLWESPELGIMKLNKACKKNFCGYKGVILEDGEWWGYTNVADSEIEYVHLNNCDDSRLPGFRIHQDRTEFEEFDIKAEMENERCMNTLSKILNKENLNFVDMSYLSPSRPGRDYAYLFEQVSWDETFCLTWPDSRKSKNCKVDWKVHKNAGLVTKKHGAIGTYYRSMCMYYPIEDTNKDGILQKDELKDKGIPGKNRYRTLKRSKNDYGEDSEFNITYNGMVVVNESFHMAVKSIYDGTEDYNSLLKFEVSEFDKIDLNEAYKEEENKWNDIDLTPVSSVNRSRSDIIKEVEKGGRKIISAVTGWFTGLAKTVRWTIWGIGSIVTIYAIWKLKKMITKKNKEDKNLVNHNELNEAFEMSKDVERGRVETWIRKNKGKEEGIYEQVSDIEDNVSKYERGVHASKGGDKMNVYSPHGKNGKKGFFNH

SEQ ID NO: 97 - protein, 551 aa, synthetic construct
MAGFYLLALTLVCYLTLVESDGTPAPVVKNETGDFLWGLGTELILPVEVISKWVEINPEDMRCPSDPVTDHVIGAIVGHTRFSRYSGFHKATQKGFLCHKMRWITACTTTWYFSHDVQRRVEAQEPGTQECLDQVKKRGEGTEEEGSYPPSQCAWNSVNEEAEVVIHLTVHEVRVDPYTMELLDPVFPSGRCNTTACDTVHQSVLWIEDPSKTRPSCGDRVVDEGNIIRSGWTDNVPGAYYLISQHLPPTKIDGACRLSFCGESGLLFENGIWTHDIKLETVVANQTFQNCQADRRAGAVGPTYEIDKLRFDWEAAKEKIKCLDMIEIISATGALSFRRLHHFNPRTPGIHPVYRIVNKTLQMAKAHYVVTNNPMSHKGTRDCLGTYMDHDTRKCVAWHHWVDVGNGTEQGPNGLLVTGDKVSYPHWFIREKTWDPALHIINYLQNADHPIVSHLGRYIDNASRDSLRKDRSENVGDAASAAVTKLAGSIGGVFKDVWHVITTCVTVGVIIIIILIFRRMIGVFWRGREKHPLPKTPNNIYQETHELKSFG

SEQ ID NO: 98 - protein, 548 aa, synthetic construct
MKKTTRHYPTINMYANLVHFTLLVASVQCVTKPSQYRNLLLPIKVHSDWKPVEIHNLTCPKPRLDWTIDFHQLATEQVYRLKDLSLGEVKGYLCHKAQWITRCEYRWYLSKVVSRRVKEETPTQVECEEAIHQFDEGREMSGSFPPEACFWNAINDESETKIILTVHDLTYDPYTDMGLDSNLVGGTCKSRFCKTTHSSVMWIKDPKSQTEPCVHMVAEKLTILSSKQGETGKRWVKTTATPAMSLEKACKLRYCGIEGILLSSGLWFGLPDSSREHHDNLGIRDNCPTTSEVGVLQPDYLDNSMEFKLEDLQREILCLQLLDKIKLQRLISMFDLQYLRPHNSGFGHVYQVINQTLMSTHAHYAVTTYPGSNSLTRSCLGQYKDSSMNTVCVNWSSWLPMKDGVYQGFNGIIEKDGVILFPEDQLLESSWSPDMFDYIPLDKIHHPFYFNMSEIIHDDIDERLIHDNSTNPGDAISDWVSVANNKVVHFFKQLGESLFIIILIILGANLTYRCVKLCIRWRLGLKSNKTQFAEEGHAMHMARESVFG

SEQ ID NO: 99 - protein, 612 aa, synthetic construct
MELIKIHLMLISFFTSSAGLERGSVGSISLISTEKLNLSTSHHDKGIFQNREKPLGHEVVLPVHCTGEWVEKPAMSLACPKRKINGPDGYYDEYFAKMWHPPTHASPEVKGYLCQKTTWNAKCEETWYFSTSKSTTIDETPINEDDCRAALILYKTGELLEPFFPPFSCYWNNININSKTFVTIHEHPTVLDLYKDTKKDPIFLHGECDGEVCETVHSNVLWVEAPLEERDDFCDPALWESSNVYTEKGGPQIPIVLESDVYGPRYTQGMCWMRICGVWGFRFSSGEWWGFRMVNKDFQGLWYTGKIPSCYGDFEVSFAHEAIAMTQLLENLSYKDHKCVDVLSTLRGNKVINAYELSYLVPEHPGFGPAYRILMVKNKRNVTQPVFILQTKNCRYQRAYLTNLSFTITGEETSESVKVGVWGDNQPVYLNWTEIGVNSTYKPNITGNWHQLMTFNGLMRFDKTLLFPQSVFVDAPNVSLLLDGFQLDLIEHPHQYFGRDEKTPSSLYKFYPHGNSTNVGEVIEGWFKSAKNAIGSLFSGMSSLMWWIISTVLSLITLLVCYRCGLLSLVKRMFTKRTRPSKKGRTSRSSHRMEHIYTEPNNPSSSSNPFFA

SEQ ID NO: 100 - protein, 602 aa, synthetic construct
MFPLSTVLLPMLFSHVQSWTHDVGRDYIHRPHNPDWFDNVISFPTECYTDWTLVRAHEIKCPSLSSINLDDKLSFKLGTVMHPLPNNKYTVDGYICHKQQWISKCEETWYFSTTETNSIENLPIGATECMEAITVYESGEYNNPFFPPFYCSWCSTQIDQKTFIVIEEHTAQENIYNASYIDPMFVGGKCSSNLCKTIHPDVLWVAKREEIRRDACNRKTWETGDVYGLVEEKKFDNDQRDFGIGEQWIRSSIYGVRRLDGSCYSKICGQFGIRFNTGEWWGLDGQGVKIWLRKILKPCQGGLRISFHHDNHDETMAIAHSVAREVTCEEVTGRILNQGFISAFDLAYLNPLNPGRGNVYRVFKRVIKGRHADEQYEYKMEKKYCMYRTLHNVSDVINQTRGKFLLGYFFDGSPFYLNQSDFEGAGKYGNERNTSRDGWFLLTYAGLTKFQQTLYTNEGVSNSQAALRNLHDPGRLALAEETEIPNVRDQMDLANKVYNSWFKMNTTSVGERITTFINNAKSAVSNYFSQLTNVIWWVGTGAIGLIVVILGRRFYRYRKSSKPPALPKRLDSVETQSTHIYEPVRSPQPVARGNQGHPFFSF

SEQ ID NO: 101 - protein, 630 aa, synthetic construct
MSNNKMFKPMIIFALILCLIINVSSYIYSGSVGGYRPKKVRTYGEVIPYTEIVEHRNLYQNWNGKHRTGHRTVLPTHCHTEWKDVTHGSIKCPHRKVIGTDGIYNTYIGDVWHPHTDSGSEIKGFLCQKTRWVSTCIETWYFSTTKETKIEEVVINPEDCLASITLMDSGEYIEPFFPPHVCSWAATNENAKEFVTVHSHPVVVDIYKNEMIDPIFLSGKCKEKVCHTIHKNVIWIEANDNERSDICVASAWESSHVFADLDIEPDIRAQKVEYIGESIDSEIYGPRSLKGACLIKICGIWGIRFSHGEWWGLKTLSNKISFADGLYNCGSNTSVGFIHNIWTPSGLIGEITYRDHKCLDVVSSLLGHQKINPYELSYLIQDFPGEGPAYRIMKQMTGLNRTKSSFKMQMKTCRYHTAYITNVTFQPIDSTQEVYKLGIWGSGHRIILNSTEIGINPTYTNSGSDWELLLTFNGLMRFGSELVLPHAVFSDHPNTSDLLEDYEINLIGHPKEIFQSDQDELAQIYKFYRRANSTNVVSLASNFLKDIGKSIGNFFGGTKNLIWWIVTLALSTLGTFIAYKLGLFKCLKRMILETDNESGNKRISNVYEEPLQLGERGHKPVKNPFFDHGI

SEQ ID NO: 102 - protein, 629 aa, synthetic construct
MTRLSHAITKLLLLFCLTAIHAIVINYPTACHTYQEVLYQGLECPEPAISYKLDNNETVAYGQICRPQLASKDILEGYLCYKDTYISSCEETWYFTSQVKQTIVHEHVSDAECIESLAYYKSGIVETPMFLNVDCYWNAINSIKKSYLIIVYHPVPFDPYTNSIKDAVVKNSEDVNSWIRDTHYPFTKWIRDFNGTAEEKCDAQHWECFKVNLYKGWIYSPPHTKNTIGSSTQTGLILESDIYSHTLIRDLCRFQFCGIHGFVFQDQSWWDLQLNVSLSSLISTEHLSGAPDGHCKKVNEIGHAELEPNWEKILSVDDYDIRHQLCLDTLASVLGGGFLTARDLLKFAPMRPGLGPAYFLFNPNKRERAVHVWTAGATTSSILWKSTCKYELIDIPQLNDTGIITYEKLDNIIGKILRNDVGVSFKDLGFTENELTDDDVSQSQLNSSLGIYHRNTSMKGIPWKRHRASTPKLKMGPNGILHDLNAKIIHLPQASSSVFKLPPHLYEGHRVVFFNHITKKKIYEDLSKREGNDPYNVDIGDLIGRHLNRTTIPDQLHDWVSGIKRHIFSVFEQFGSLIKVVVFIIMLVLCIKIINLIYRFYKVRKSNHKKLASRKEKLHLSDPFSVNSK

SEQ ID NO: 103 - protein, 623 aa, synthetic construct
MFKVLIITLLVNKIHLEKIYNVPVNCGELHPVKAHEIKCPQRLNELSLQAHHNLAKDEHYNKICRPQLKDDAHLEGFICRKQRWITKCSETWYFSTSIEYQILEVIPEYSGCTDAVKKLDQGALIPPYYPPAGCFWNTEMNQEIEFYVLIQHKPFLNPYDNLIYDSRFLTPCTINDSKTKGCPLKDITGTWIPDVRVEEISEHCNNKHWECITVKSFRSELNDKERLWEAPDIGLVHVNKGCLSTFCGKNGIIFEDGEWWSIENQTESDFQNFKIEKCKGKKPGFRMHTDRTEFEELDIKAELEHERCLNTISKILNKENINTLDMSYLAPTRPGRDYAYLFEQTSWQEKLCLSLPDSGRVSKDCNIDWRTSTRGGMVKKNHYGIGSYKRAWCEYRPFVDKNEDGYIDIQELNGHNMSGNHAILETAPAGGSSGNRLNVTLNGMIFVEPTKLYLHTKSLYEGIEDYQKLIKFEVMEYDNVEENLIRYEEDEKFKPVNLNPHEKSQINRTDIVREIQKGGKKVLSAVVGWFTSTAKAVRWTIWAVGAIVTTYAIYKLYKMVKSNSSHSKHREADLEGLQSTTKENMRVEKNDKNYQDLELGLYEEIRSIKGGSKQTGDDRFFDH

SEQ ID NO: 104 - protein, 820 aa, synthetic construct
MDLVRFSIALSVFLCYGTPPSQGQAIVSIKDSCEAKSAPWIPCEKFDYVKNATGSGIKCWIFCSRSGFYSKTGRFIRCIQGDPEAKYIKSCRRQIEKRGKEKMREGTRGKRKTSEPKEEGVRAKTDFTPDESRRLNNLTKVFRKVEDKDLNDFKKFILEKGLETKIKLANDGKISFRDPDCGENKDYPCHRIHQIIEGVNENIDYINEILSLKKMKEELRLRERESEEGEFPGLLNTTNRRGFLLHYPVELGNWSRLEDPSQIKCPSHHKDMLSNPRRLGKYNLDIIVRRPRIGTFETVVPGYICQGMQWTSTCNEMWYFVTYHDRAVHYITPNKLKCLQNIRAHKRGEHIKPYYPLEECNWNSETTKTVDYFMITPYSPEVDPFTLEFKSEIFPDRTSCRPGDEICVTDDDSKVWFPDEDDKLIARGHCPDETWDESHLTIHPEEMPENWEDPQSPWVSDYILKGVLFGEKRVKKSCLLEFCGTSGLLFEDGEWWELNVFSREKGRESLTKIFIEQEEIRRCNGTETRVGVAGKETDEKALLNAVLSKNAYERCKSARYRLIENKYLRLDDLSYINPRESVTWWAYRVRAGDDERTFKLEKTTGEYRYLQVPPSLEQHVTDCDGQENCSVSIGYYRGELINSSDWTRTGHDDVYVGVNGLLRKDTGNKTIVLYPPLMKEYQEIFSDSGESDDEAFIYKPDIHEKKGKPKEAEDEKDEKSKKNKTPIDDIKDWWSNIKGEWHLIKGILIGLFTFALLIGVVKLGVFIKSSFRKRRDDSIPEGKDEEIGIKMQSRRSRQNIYEEINEVSPTMTRRGRNIFN

SEQ ID NO: 105 - protein, 561 aa, synthetic construct
MAHYELHVLFVHSWMLALILITTLVWLAASQKAFTPDLVFPEMNRNSSWSVANYGEILCPTSFQSYDPKKHQILTRVLVERPSLNTDTKVEGYTCHKVKYETICDMPWYFSPTISHSISPLRVKESECKDAIAEHQLGTHVPLSFPPEDCSWNSVNTKEYEDIIVKEHPVMLDPYTNNYVDAIFPGGISSPGMGGTIHDDMMWVSKDLAVSPECSGWQRSMGLIYSSRLYGEREPMLEVGSIHIEGHRDKNLTLACRISFCGEIGVRFHDGEWMKVSVNLDHPNSVTFQVTDFPPCPPGTTIQTAVVENINPEIQELTVNMMYRLKCQETISKMVSGLPTSALDLSYLIQVQEGPGIVYKREKGILYQSVGMYQYIDTVTLNKEENQLGENSRGQKVFWTEWSDSPTRPGLQEGINGIVKYEGQVRVPLGMSLRLEAATELMWGHPVHTVSHPILHVISNHTEQSVTTWNRGVNSTNLIGLATRSISGFYDNLKLYLILALIFVSLIALVVLDVIPFKYILFVLCPPLLLCRFIKCSRRKPETRDRYHVEYNRPGQVSSAF

SEQ ID NO: 106 - protein, 511 aa, synthetic construct
MSQGYGLLFLVSGVLSLTAGYVFHYPIEKDIHWYPANHSSLRCPIRSASITDTPTGGVTISIPSNPSNNDLPGFSCHKTEWISECTETWYWSTDVKQYIRPVSVTADECKKAQRDKEVGTEITPFFTAPVCQWSNTVRKVNSFVITNKKNVKFDPYNLDFIDPILVGGRCKGNQESCPTIQAGVIWLPRLQPTKATSWTNIYAKYKRVGPHMGDWKFWGGGMPTSTFKDACKMEFRGKEGIRVSSGFWFHIPQMDDVEFKTEYGKLAHCVSSKEIKFPSAHEEVAEHEMEIQDLILTLRCRDIIDKYEETGSISFMDLALFDPDNEGPAHIYRINKGKLEAGLVNYGECKVSKKGDPAESACVKVMDNGQRSPIFFQDWVPTGIKGIQSGFNGLYRENGEIKHAGYNLFQNKLTESDIQRMELTPIHHPVLLSLSDVAPGLNVTFDQTGERGELDLDLLPGITGIWRKFVEYLSMAALILTLIVSIFVVWKCCISNHLGPSKKTSEMEYFE

SEQ ID NO: 107 - protein, 470 aa, synthetic construct
MITHKYILPIIVLSNFMPVKRDDLTCPIYNHQNVEFVNTTITYIRLNSVLLQSKSLELKQMPYVRGHICQKIRYITTCKANLFTSNEIFYKKEYLTVTKNDCEMKAVHETGEYPAPICTWSLFGSNLHNEEIIVTEIQSHDYHYDLFNGKIKDSEYLFEHCTLMYCKLREHKGYWIKSEPTKNDICPIVQDDEIVAELKFYNENHFLKINHHLYSTEEVCKIKYCNNSLLFFKDIGFFNIKSTLQKMDKIFKKCTKIEDLRYIIHDNVEKIDNLNDCLNFKLNVLTNKDRTIAYHDIRKLHPKGTGINRIYRLNGNNTLESAIAYYGSVESNETKIYLDYWNDCSKTHTCTYNGYMGKKGLNIRARLNIDLFQEVYEEDSSLLIYNGTTDLSTGTIGELTKGDANVTFTTKEWIPHISNYIIIIVFCLLSGAVFIIMINIYNRIMVMKRNRKAFYNRENDNRVIYVNDWK

SEQ ID NO: 108 - protein, 531 aa, synthetic construct
MAPQTISLLWAMVCVSVYTRANRVVAPIHEPQNWKPATVDDFTCRTGFNLDFDSKFIKTKALVLKRVGQAKVKGYLCMKNRWTTTCETNWLYSKSVSHHITHVAVSAEECYNKIRDDASGNLKIESYPNPQCAWSSTVSREEDFIHISTSDVGYDMYTDTVLSPSFPGGTCKLKTCCKTIYPNIVWVPETPAQTQVRDALFDETMVTVTVEAKKVVKDSWVTGATITPSVMEGSCKKTLGSKSGILLPNGQWFSIVETGQITIQPKGSVEEKETWVNLINDLNLSDCAETQEAKVPTAEFTVYKTESMVFNILNYHLCLETVAKARSGKNLTRLDLARLAPEIPGVAHVYQLTSDGVRVGSTRYEIIAWKPTMGLDKTLGLTIVPSGNRNSETIKWIEWTRTDDGLLNGPNGIFIADGKEIVHPNLKMVSFELETYLISEHSTQLVPHPVIHSISDEIYPENYTIGGKNSYIKIHTPTAYFWSGIHWIEGAVQKLFIVVVATALIGLFILVVWLCCGCCSKSRPVRNQKWE

SEQ ID NO: 109 - protein, 16 aa, synthetic construct
MKCLLYLAFLFIGVNC

SEQ ID NO: 110 - protein, 22 aa, synthetic construct
MSYLIVKVVILFLVGIDKQVLS

SEQ ID NO: 111 - protein, 21 aa, synthetic construct
MTSSVTISVILLISFIAPSYS

SEQ ID NO: 112 - protein, 20 aa, synthetic construct
MTSVLFMVGVLLGAFGSTHC

SEQ ID NO: 113 - protein, 20 aa, synthetic construct
MESLPFSALLAVLSITLCDS

SEQ ID NO: 114 - protein, 15 aa, synthetic construct
MNVWRFLSLVPLATS

SEQ ID NO: 115 - protein, 19 aa, synthetic construct
MESLLKAICVLLLIHCSRC

SEQ ID NO: 116 - protein, 19 aa, synthetic construct
MSSKIVLAAICLCSVQYVA

SEQ ID NO: 117 - protein, 18 aa, synthetic construct
MDLFPILVVVLMTDTVLG

SEQ ID NO: 118 - protein, 18 aa, synthetic construct
MISITFVYLIIILSLSWG

SEQ ID NO: 119 - protein, 17 aa, synthetic construct
MYAQYITISLFALQAHS

SEQ ID NO: 120 - protein, 17 aa, synthetic construct
MISSTLILVIISAHAFC

SEQ ID NO: 121 - protein, 15 aa, synthetic construct
MWSAVISSIIPLVLS

SEQ ID NO: 122 - protein, 16 aa, synthetic construct
MDFWMIIYVLVVSASA

SEQ ID NO: 123 - protein, 24 aa, synthetic construct
MILIRWFLWISLSLLLLGINEVDG

SEQ ID NO: 124 - protein, 19 aa, synthetic construct
MECIMFWTILALSISFGFS

SEQ ID NO: 125 - protein, 22 aa, synthetic construct
MSSEIKMLIELMLFSILSCVIS

SEQ ID NO: 126 - protein, 20 aa, synthetic construct
MAGFYLLALTLVCYLTLVES

SEQ ID NO: 127 - protein, 29 aa, synthetic construct
MKKTTRHYPTINMYANLVHFTLLVASVQC

SEQ ID NO: 128 - protein, 19 aa, synthetic construct
MELIKIHLMLISFFTSSAG

SEQ ID NO: 129 - protein, 18 aa, synthetic construct
MFPLSTVLLPMLFSHVQS

SEQ ID NO: 130 - protein, 25 aa, synthetic construct
MSNNKMFKPMIIFALILCLIINVSS

SEQ ID NO: 131 - protein, 22 aa, synthetic construct
MTRLSHAITKLLLLFCLTAIHA

SEQ ID NO: 132 - protein, 12 aa, synthetic construct
MFKVLIITLLVN

SEQ ID NO: 133 - protein, 26 aa, synthetic construct
MDLVRFSIALSVFLCYGTPPSQGQAI

SEQ ID NO: 134 - protein, 30 aa, synthetic construct
MAHYELHVLFVHSWMLALILITTLVWLAAS

SEQ ID NO: 135 - protein, 21 aa, synthetic construct
MSQGYGLLFLVSGVLSLTAGY

SEQ ID NO: 136 - protein, 20 aa, synthetic construct
MITHKYILPIIVLSNFMPVK

SEQ ID NO: 137 - protein, 21 aa, synthetic construct
MAPQTISLLWAMVCVSVYTRA

SEQ ID NO: 138 - protein, 229 aa, Vesiculovirus indiana
MSSLKKILGLKGKGKKSKKLGIAPPPYEEDTSMEYAPSAPIDKSYFGVDEMDTYDPNQLRYEKFFFTVKMTVRSNRPFRTYSDVAAAVSHWDHMYIGMAGKRPFYKILAFLGSSNLKATPAVLADQGQPEYHAHCEGRAYLPHRMGKTPPMLNVPEHFRRPFNIGLYKGTIELTMTIYDDESLEAAPMIWDHFNSSKFSDFREKALMFGLIVEKKASGAWVLDSIGHFK

SEQ ID NO: 139 - DNA, 690 bp, Vesiculovirus indiana
  source 1..690: mol_type = genomic DNA
atgagttccttaaagaagattctcggtctgaaggggaaaggtaagaaatctaagaaattagggatcgcaccacccccttatgaagaggacactagcatggagtatgctccgagcgctccaattgacaaatcctattttggagttgacgagatggacacctatgatccgaatcaattaagatatgagaaattcttctttacagtgaaaatgacggttagatctaatcgtccgttcagaacatactcagatgtggcagccgctgtatcccattgggatcacatgtacatcggaatggcagggaaacgtcccttctacaaaatcttggcttttttgggttcttctaatctaaaggccactccagcggtattggcagatcaaggtcaaccagagtatcacgctcactgcgaaggcagggcttatttgccacataggatggggaagacccctcccatgctcaatgtaccagagcacttcagaagaccattcaatataggtctttacaagggaacgattgagctcacaatgaccatctacgatgatgagtcactggaagcagctcctatgatctgggatcatttcaattcttccaaattttctgatttcagagagaaggccttaatgtttggcctgattgtcgagaaaaaggcatctggagcgtgggtcctggactctatcggccacttcaaatga

SEQ ID NO: 140 - protein, 229 aa, synthetic construct
MSSLKKILGLKGKGKKSKKLGIAPPPYEEDTSMEYAPSAPIDKSYFGVDERDTYDPNQLRYEKFFFTVKMTVRSNRPFRTYSDVAAAVSHWDHMYIGMAGKRPFYKILAFLGSSNLKATPAVLADQGQPEYHAHCEGRAYLPHRMGKTPPMLNVPEHFRRPFNIGLYKGTIELTMTIYDDESLEAAPMIWDHFNSSKFSDFREKALMFGLIVEKKASGAWVLDSIGHFK

SEQ ID NO: 141 - DNA, 690 bp, synthetic construct
  source 1..690: mol_type = other DNA
atgagttccttaaagaagattctcggtctgaaggggaaaggtaagaaatctaagaaattagggatcgcaccacccccttatgaagaggacactagcatggagtatgctccgagcgctccaattgacaaatcctattttggagttgacgagcgagacacctatgatccgaatcaattaagatatgagaaattcttctttacagtgaaaatgacggttagatctaatcgtccgttcagaacatactcagatgtggcagccgctgtatcccattgggatcacatgtacatcggaatggcagggaaacgtcccttctacaaaatcttggcttttttgggttcttctaatctaaaggccactccagcggtattggcagatcaaggtcaaccagagtatcacgctcactgcgaaggcagggcttatttgccacataggatggggaagacccctcccatgctcaatgtaccagagcacttcagaagaccattcaatataggtctttacaagggaacgattgagctcacaatgaccatctacgatgatgagtcactggaagcagctcctatgatctgggatcatttcaattcttccaaattttctgatttcagagagaaggccttaatgtttggcctgattgtcgagaaaaaggcatctggagcgtgggtcctggactctatcggccacttcaaatga

SEQ ID NO: 142 - protein, 228 aa, synthetic construct
MSSLKKILGLKGKGKKSKKLGIAPPPYEEDTSMEYAPSAPIDKSYFGVDEDTYDPNQLRYEKFFFTVKMTVRSNRPFRTYSDVAAAVSHWDHMYIGMAGKRPFYKILAFLGSSNLKATPAVLADQGQPEYHAHCEGRAYLPHRMGKTPPMLNVPEHFRRPFNIGLYKGTIELTMTIYDDESLEAAPMIWDHFNSSKFSDFREKALMFGLIVEKKASGAWVLDSIGHFK

SEQ ID NO: 143 - DNA, 687 bp, synthetic construct
  source 1..687: mol_type = other DNA
atgagttccttaaagaagattctcggtctgaaggggaaaggtaagaaatctaagaaattagggatcgcaccacccccttatgaagaggacactagcatggagtatgctccgagcgctccaattgacaaatcctattttggagttgacgaggacacctatgatccgaatcaattaagatatgagaaattcttctttacagtgaaaatgacggttagatctaatcgtccgttcagaacatactcagatgtggcagccgctgtatcccattgggatcacatgtacatcggaatggcagggaaacgtcccttctacaaaatcttggcttttttgggttcttctaatctaaaggccactccagcggtattggcagatcaaggtcaaccagagtatcacgctcactgcgaaggcagggcttatttgccacataggatggggaagacccctcccatgctcaatgtaccagagcacttcagaagaccattcaatataggtctttacaagggaacgattgagctcacaatgaccatctacgatgatgagtcactggaagcagctcctatgatctgggatcatttcaattcttccaaattttctgatttcagagagaaggccttaatgtttggcctgattgtcgagaaaaaggcatctggagcgtgggtcctggactctatcggccacttcaaatga

SEQ ID NO: 144 - DNA, 14311 bp, synthetic construct
  source 1..14311: mol_type = other DNA
cacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtaccgggccccccctcgagttgtaatacgactcactatagggacgaagacaaacaaaccattattatcattaaaaggctcaggagaaactttaacagtaatcaaaatgtctgttacagtcaagagaatcattgacaacacagtcatagttccaaaacttcctgcaaatgaggatccagtggaatacccggcagattacttcagaaaatcaaaggagattcctctttacatcaatactacaaaaagtttgtcagatctaagaggatatgtctaccaaggcctcaaatccggaaatgtatcaatcatacatgtcaacagctacttgtatggagcattaaaggacatccggggtaagttggataaagattggtcaagtttcggaataaacatcgggaaagcaggggatacaatcggaatatttgaccttgtatccttgaaagccctggacggcgtacttccagatggagtatcggatgcttccagaaccagcgcagatgacaaatggttgcctttgtatctacttggcttatacagagtgggcagaacacaaatgcctgaatacagaaaaaagctcatggatgggctgacaaatcaatgcaaaatgatcaatgaacagtttgaacctcttgtgccagaaggtcgtgacatttttgatgtgtggggaaatgacagtaattacacaaaaattgtcgctgcagtggacatgttcttccacatgttcaaaaaacatgaatgtgcctcgttcagatacggaactattgtttccagattcaaagattgtgctgcattggcaacatttggacacctctgcaaaataaccggaatgtctacagaagatgtaacgacctggatcttgaaccgagaagttgcagatgaaatggtccaaatgatgcttccaggccaagaaattgacaaggccgattcatacatgccttatttgatcgactttggattgtcttctaagtctccatattcttccgtcaaaaaccctgccttccacttctgggggcaattgacagctcttctgctcagatccaccagagcaaggaatgcccgacagcctgatgacattgagtatacatctcttactacagcaggtttgttgtacgcttatgcagtaggatcctctgccgacttggcacaacagttttgtgttggagataacaaatacactccagatgatagtaccggaggattgacgactaatgcaccgccacaaggcagagatgtggtcgaatggctcggatggtttgaagatcaaaacagaaaaccgactcctgatatgatgcagtatgcgaaaagagcagtcatgtcactgcaaggcctaagagagaagacaattggcaagtatgctaagtcagaatttgacaaatgaccctataattctcagatcacctattatatattatgctacatatgaaaaaaactaacagatatcatggataatctcacaaaagttcgtgagtatctcaagtcctattctcgtctggatcaggcggtaggagagatagatgagatcgaagcacaacgagctgaaaagtccaattatgagttgttccaagaggatggagtggaagagcatactaagccctcttattttcaggcagcagatgattctgacacagaatctgaaccagaaattgaagacaatcaaggtttgtatgcaccagatccagaagctgagcaagttgaaggctttatacaggggcctttagatgactatgcagatgaggaagtggatgttgtatttacttcggactggaaacagcctgagcttgaatctgacgagcatggaaagaccttacggttgacatcgccagagggtttaagtggagagcagaaatcccagtggctttcgacgattaaagcagtcgtgcaaagtgccaaatactggaatctggcagagtgcacatttgaagcatcgggagaaggggtcattatgaaggagcgccagataactccggatgtatataaggtcactccagtgatgaacacacatccgtcccaatcagaagcagtatcagatgtttggtctctctcaaagacatccatgactttccaacccaagaaagcaagtcttcagcctctcaccatatccttggatgaattgttctcatctagaggagagttcatctctgtcggaggtgacggacgaatgtctcataaagaggccatcctgctcggcctgagatacaaaaagttgtacaatcaggcgagagtcaaatattctctgtagactatgaaaaaaagtaacagatatcacgatctaagtgttatcccaatccattcatcatgagttccttaaagaagattctcggtctgaaggggaaaggtaagaaatctaagaaattagggatcgcaccacccccttatgaagaggacactagcatggagtatgctccgagcgctccaattgacaaatcctattttggagttgacgagatggacacctatgatccgaatcaattaagatatgagaaattcttctttacagtgaaaatgacggttagatctaatcgtccgttcagaacatactcagatgtggcagccgctgtatcccattgggatcacatgtacatcggaatggcagggaaacgtcccttctacaaaatcttggcttttttgggttcttctaatctaaaggccactccagcggtattggcagatcaaggtcaaccagagtatcacactcactgcgaaggcagggcttatttgccacataggatggggaagacccctcccatgctcaatgtaccagagcacttcagaagaccattcaatataggtctttacaagggaacgattgagctcacaatgaccatctacgatgatgagtcactggaagcagctcctatgatctgggatcatttcaattcttccaaattttctgatttcagagagaaggccttaatgtttggcctgattgtcgagaaaaaggcatctggagcgtgggtcctggattctatcagccacttcaaatgagctagtctaacttctagcttctgaacaatccccggtttactcagtctctcctaattccagcctctcgaacaactaatatcctgtcttttctatccctatgaaaaaaactaacagagatcgatctgtttacgcgtcactatgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccatacaagtcaaaatgcccaagagtcacaaggctattcaagcagacggttggatgtgtcatgcttccaaatgggtcactacttgtgatttccgctggtatggaccgaagtatataacacagtccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaaagggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaagtaactcaaatcctgctagccagattcttcatgtttggaccaaatcaacttgtgataccatgctcaaagaggcctcaattatatttgagtttttaatttttatgaaaaaaactaacagcaatcatggaagtccacgattttgagaccgacgagttcaatgatttcaatgaagatgactatgccacaagagaattcctgaatcccgatgagcgcatgacgtacttgaatcatgctgattacaatttgaattctcctctaattagtgatgatattgacaatttgatcaggaaattcaattctcttccgattccctcgatgtgggatagtaagaactgggatggagttcttgagatgttaacatcatgtcaagccaatcccatctcaacatctcagatgcataaatggatgggaagttggttaatgtctgataatcatgatgccagtcaagggtatagttttttacatgaagtggacaaagaggcagaaataacatttgacgtggtggagaccttcatccgcggctggggcaacaaaccaattgaatacatcaaaaaggaaagatggactgactcattcaaaattctcgcttatttgtgtcaaaagtttttggacttacacaagttgacattaatcttaaatgctgtctctgaggtggaattgctcaacttggcgaggactttcaaaggcaaagtcagaagaagttctcatggaacgaacatatgcaggattagggttcccagcttgggtcctacttttatttcagaaggatgggcttacttcaagaaacttgatattctaatggaccgaaactttctgttaatggtcaaagatgtgattatagggaggatgcaaacggtgctatccatggtatgtagaatagacaacctgttctcagagcaagacatcttctcccttctaaatatctacagaattggagataaaattgtggagaggcagggaaatttttcttatgacttgattaaaatggtggaaccgatatgcaacttgaagctgatgaaattagcaagagaatcaaggcctttagtcccacaattccctcattttgaaaatcatatcaagacttctgttgatgaaggggcaaaaattgaccgaggtataagattcctccatgatcagataatgagtgtgaaaacagtggatctcacactggtgatttatggatcgttcagacattggggtcatccttttatagattattacactggactagaaaaattacattcccaagtaaccatgaagaaagatattgatgtgtcatatgcaaaagcacttgcaagtgatttagctcggattgttctatttcaacagttcaatgatcataaaaagtggttcgtgaatggagacttgctccctcatgatcatccctttaaaagtcatgttaaagaaaatacatggcccacagctgctcaagttcaagattttggagataaatggcatgaacttccgctgattaaatgttttgaaatacccgacttactagacccatcgataatatactctgacaaaagtcattcaatgaataggtcagaggtgttgaaacatgtccgaatgaatccgaacactcctatccctagtaaaaaggtgttgcagactatgttggacacaaaggctaccaattggaaagaatttcttaaagagattgatgagaagggcttagatgatgatgatctaattattggtcttaaaggaaaggagagggaactgaagttggcaggtagatttttctccctaatgtcttggaaattgcgagaatactttgtaattaccgaatatttgataaagactcatttcgtccctatgtttaaaggcctgacaatggcggacgatctaactgcagtcattaaaaagatgttagattcctcatccggccaaggattgaagtcatatgaggcaatttgcatagccaatcacattgattacgaaaaatggaataaccaccaaaggaagttatcaaacggcccagtgttccgagttatgggccagttcttaggttatccatccttaatcgagagaactcatgaattttttgagaaaagtcttatatactacaatggaagaccagacttgatgcgtgttcacaacaacacactgatcaattcaacctcccaacgagtttgttggcaaggacaagagggtggactggaaggtctacggcaaaaaggatggactatcctcaatctactggttattcaaagagaggctaaaatcagaaacactgctgtcaaagtcttggcacaaggtgataatcaagttatttgcacacagtataaaacgaagaaatcgagaaacgttgtagaattacagggtgctctcaatcaaatggtttctaataatgagaaaattatgactgcaatcaaaatagggacagggaagttaggacttttgataaatgacgatgagactatgcaatctgcagattacttgaattatggaaaaataccgattttccgtggagtgattagagggttagagaccaagagatggtcacgagtgacttgtgtcaccaatgaccaaatacccacttgtgctaatataatgagctcagtttccacaaatgctctcaccgtagctcattttgctgagaacccaatcaatgccatgatacagtacaattattttgggacatttgctagactcttgttgatgatgcatgatcctgctcttcgtcaatcattgtatgaagttcaagataagataccgggcttgcacagttctactttcaaatacgccatgttgtatttggacccttccattggaggagtgtcgggcatgtctttgtccaggtttttgattagagccttcccagatcccgtaacagaaagtctctcattctggagattcatccatgtacatgctcgaagtgagcatctgaaggagatgagtgcagtatttggaaaccccgagatagccaagtttcgaataactcacatagacaagctagtagaagatccaacctctctgaacatcgctatgggaatgagtccagcgaacttgttaaagactgaggttaaaaaatgcttaatcgaatcaagacaaaccatcaggaaccaggtgattaaggatgcaaccatatatttgtatcatgaagaggatcggctcagaagtttcttatggtcaataaatcctctgttccctagatttttaagtgaattcaaatcaggcacttttttgggagtcgcagacgggctcatcagtctatttcaaaattctcgtactattcggaactcctttaagaaaaagtatcatagggaattggatgatttgattgtgaggagtgaggtatcctctttgacacatttagggaaacttcatttgagaaggggatcatgtaaaatgtggacatgttcagctactcatgctgacacattaagatacaaatcctggggccgtacagttattgggacaactgtaccccatccattagaaatgttgggtccacaacatcgaaaagagactccttgtgcaccatgtaacacatcagggttcaattatgtttctgtgcattgtccagacgggatccatgacgtctttagttcacggggaccattgcctgcttatctagggtctaaaacatctgaatctacatctattttgcagccttgggaaagggaaagcaaagtcccactgattaaaagagctacacgtcttagagatgctatctcttggtttgttgaacccgactctaaactagcaatgactatactttctaacatccactctttaacaggcgaagaatggaccaaaaggcagcatgggttcaaaagaacagggtctgcccttcataggttttcgacatctcggatgagccatggtgggttcgcatctcagagcactgcagcattgaccaggttgatggcaactacagacaccatgagggatctgggagatcagaatttcgactttttattccaagcaacgttgctctatgctcaaattaccaccactgttgcaagagacggatggatcaccagttgtacagatcattatcatattgcctgtaagtcctgtttgagacccatagaagagatcaccctggactcaagtatggactacacgcccccagatgtatcccatgtgctgaagacatggaggaatggggaaggttcgtggggacaagagataaaacagatctatcctttagaagggaattggaagaatttagcacctgctgagcaatcctatcaagtcggcagatgtataggttttctatatggagacttggcgtatagaaaatctactcatgccgaggacagttctctatttcctctatctatacaaggtcgtattagaggtcgaggtttcttaaaagggttgctagacggattaatgagagcaagttgctgccaagtaatacaccggagaagtctggctcatttgaagaggccggccaacgcagtgtacggaggtttgatttacttgattgataaattgagtgtatcacctccattcctttctcttactagatcaggacctattagagacgaattagaaacgattccccacaagatcccaacctcctatccgacaagcaaccgtgatatgggggtgattgtcagaaattacttcaaataccaatgccgtctaattgaaaagggaaaatacagatcacattattcacaattatggttattctcagatgtcttatccatagacttcattggaccattctctatttccaccaccctcttgcaaatcctatacaagccatttttatctgggaaagataagaatgagttgagagagctggcaaatctttcttcattgctaagatcaggagaggggtgggaagacatacatgtgaaattcttcaccaaggacatattattgtgtccagaggaaatcagacatgcttgcaagttcgggattgctaaggataataataaagacatgagctatcccccttggggaagggaatccagagggacaattacaacaatccctgtttattatacgaccaccccttacccaaagatgctagagatgcctccaagaatccaaaatcccctgctgtccggaatcaggttgggccaattaccaactggcgctcattataaaattcggagtatattacatggaatgggaatccattacagggacttcttgagttgtggagacggctccggagggatgactgctgcattactacgagaaaatgtgcatagcagaggaatattcaatagtctgttagaattatcagggtcagtcatgcgaggcgcctctcctgagccccccagtgccctagaaactttaggaggagataaatcgagatgtgtaaatggtgaaacatgttgggaatatccatctgacttatgtgacccaaggacttgggactatttcctccgactcaaagcaggcttggggcttcaaattgatttaattgtaatggatatggaagttcgggattcttctactagcctgaaaattgagacgaatgttagaaattatgtgcaccggattttggatgagcaaggagttttaatctacaagacttatggaacatatatttgtgagagcgaaaagaatgcagtaacaatccttggtcccatgttcaagacggtcgacttagttcaaacagaatttagtagttctcaaacgtctgaagtatatatggtatgtaaaggtttgaagaaattaatcgatgaacccaatcccgattggtcttccatcaatgaatcctggaaaaacctgtacgcattccagtcatcagaacaggaatttgccagagcaaagaaggttagtacatactttaccttgacaggtattccctcccaattcattcctgatccttttgtaaacattgagactatgctacaaatattcggagtacccacgggtgtgtctcatgcggctgccttaaaatcatctgatagacctgcagatttattgaccattagccttttttatatggcgattatatcgtattataacatcaatcatatcagagtaggaccgatacctccgaaccccccatcagatggaattgcacaaaatgtggggatcgctataactggtataagcttttggctgagtttgatggagaaagacattccactatatcaacagtgtttagcagttatccagcaatcattcccgattaggtgggaggctgtttcagtaaaaggaggatacaagcagaagtggagtactagaggtgatgggctcccaaaagatacccgaacttcagactccttggccccaatcgggaactggatcagatctctggaattggtccgaaaccaagttcgtctaaatccattcaatgagatcttgttcaatcagctatgtcgtacagtggataatcatttgaaatggtcaaatttgcgaagaaacacaggaatgattgaatggatcaatagacgaatttcaaaagaagaccggtctatactgatgttgaagagtgacctacacgaggaaaactcttggagagattaaaaaatcatgaggagactccaaactttaagtatgaaaaaaactttgatccttaagaccctcttgtggtttttattttttatctggttttgtggtcttcgtgggtcggcatggcatctccacctcctcgcggtccgacctgggcatccgaaggaggacgtcgtccactcggatggctaagggaggggcccccgcggggctgctaacaaagcccgaaaggaagctgagttggctgctgccaccgctgagcaataactagcataaccccttggggcctctaaacgggtcttgaggggttttttgctgaaaggaggaactatatccggatcgagacctcgatactagtgcggtggagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgc

SEQ ID NO: 145-148 - skipped (4 entries)

SEQ ID NO: 149 - DNA, 22 bp, synthetic construct
  source 1..22: mol_type = other DNA
gtcagaatttgacaaatgaccc

SEQ ID NO: 150 - DNA, 26 bp, synthetic construct
  source 1..26: mol_type = other DNA
gtgagattatccatgatatctgttag

SEQ ID NO: 151 - DNA, 34 bp, synthetic construct
  source 1..34: mol_type = other DNA
  modified_base 34: mod_base = OTHER; note = Hexachlorofluorescein modified nucleotide
ctcgagtcacctattatatattatgctacatatg

SEQ ID NO: 152 - DNA, 19 bp, synthetic construct
  source 1..19: mol_type = other DNA
agatttggacctgcgagcg

SEQ ID NO: 153 - DNA, 20 bp, synthetic construct
  source 1..20: mol_type = other DNA
gagcggctgtctccacaagt

SEQ ID NO: 154 - DNA, 23 bp, synthetic construct
  source 1..23: mol_type = other DNA
  modified_base 23: mod_base = OTHER; note = Fluorescein modified nucleotide
ttctgacctgaaggctctgcgcg

SEQ ID NO: 155 - protein, 5 aa, synthetic construct
KFSGG

SEQ ID NO: 156 - protein, 5 aa, synthetic construct
AAQPA

SEQ ID NO: 157 - protein, 495 aa, synthetic construct
KFTIVFPHNQKGNWKNVPSNYHYCPSSSDLNWHNDLIGTAIQVKMPKSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 158 - protein, 257 aa, synthetic construct
QVQLVQSGAEVKKPGESLKISCKGSGYSFTSYWIAWVRQMPGKGLEYMGLIYPGDSDTKYSPSFQGQVTISVDKSVSTAYLQWSSLKPSDSAVYFCARHDVGYCTDRTCAKWPEWLGVWGQGTLVTVSSGGGGSGGGGSGGGGSQSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKVTVLGAK

SEQ ID NO: 159 - protein, 129 aa, synthetic construct
QVQLVQSGAEVKKPGESLKISCKGSGYSFTSYWIAWVRQMPGKGLEYMGLIYPGDSDTKYSPSFQGQVTISVDKSVSTAYLQWSSLKPSDSAVYFCARHDVGYCTDRTCAKWPEWLGVWGQGTLVTVSS

SEQ ID NO: 160 - protein, 113 aa, synthetic construct
QSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKVTVLGAK

SEQ ID NO: 161 - protein, 245 aa, synthetic construct
MAEVQLQQSGAELVKPGASVKLSCKASGYTFTSHWMHWVKQRAGQGLEWIGEFNPSNGRTNYNEKFKSKATLTVDKSSSTAYMQLSSLTSEDSAVYYCASRDYDYDGRYFDYWGQGTTVTVSSGGGGSGGGGSGGGGSDIELTQSPAIMSASPGEKVTMTCSASSSVTYMYWYQQKPGSSPRLLIYDTSNLASGVPVRFSGSGSGTSYSLTISRMEAEDAATYYCQQWSSHIFTFGSGTELEIKR

SEQ ID NO: 162 - protein, 123 aa, synthetic construct
MAEVQLQQSGAELVKPGASVKLSCKASGYTFTSHWMHWVKQRAGQGLEWIGEFNPSNGRTNYNEKFKSKATLTVDKSSSTAYMQLSSLTSEDSAVYYCASRDYDYDGRYFDYWGQGTTVTVSS

SEQ ID NO: 163 - protein, 107 aa, synthetic construct
DIELTQSPAIMSASPGEKVTMTCSASSSVTYMYWYQQKPGSSPRLLIYDTSNLASGVPVRFSGSGSGTSYSLTISRMEAEDAATYYCQQWSSHIFTFGSGTELEIKR

SEQ ID NO: 164 - protein, 27 aa, synthetic construct
RAAARGSPKGGGGSGGGGSGGGGSKFT

SEQ ID NO: 165 - protein, 22 aa, synthetic construct
RAAAGGGGSGGGGSGGGGSKFT

SEQ ID NO: 166 - protein, 23 aa, synthetic construct
RAAASGGSGGGGSGGGGSGPKFT

SEQ ID NO: 167 - protein, 20 aa, synthetic construct
RAAASGGSGGGGSGGGGSFT

SEQ ID NO: 168 - protein, 18 aa, synthetic construct
REAAAKEAAAKEAAAKFT

SEQ ID NO: 169 - protein, 24 aa, synthetic construct
RAAARGSPKGGGGSGGGGSGGGGS

SEQ ID NO: 170 - protein, 19 aa, synthetic construct
RAAAGGGGSGGGGSGGGGS

SEQ ID NO: 171 - protein, 20 aa, synthetic construct
RAAASGGSGGGGSGGGGSGP

SEQ ID NO: 172 - protein, 18 aa, synthetic construct
RAAASGGSGGGGSGGGGS

SEQ ID NO: 173 - protein, 16 aa, synthetic construct
REAAAKEAAAKEAAAK

SEQ ID NO: 174 - protein, 22 aa, synthetic construct
KRAAASGGSGGGGSGGGGSGPK

SEQ ID NO: 175 - protein, 19 aa, synthetic construct
KRGGGSGGSGGGGSGGGGS

SEQ ID NO: 176 - protein, 18 aa, synthetic construct
RGGGSGGSGGGGSGGGGS

SEQ ID NO: 177 - protein, 22 aa, synthetic construct
KRGGGSGGSGGGGSGGGGSGPK

SEQ ID NO: 178 - protein, 20 aa, synthetic construct
RGGGSGGSGGGGSGGGGSGP

SEQ ID NO: 179 - DNA, 1488 bp, synthetic construct
  source 1..1488: mol_type = other DNA
aagttcaccatagtttttccacacaaccaaaaaggaaactggaaaaatgttccttctaattaccattattgcccgtcaagctcagatttaaattggcataatgacttaataggcacagccttacaagtcaaaatgcccaagagtcacaaggctattcaagcagacggttggatgtgtcatgcttccaaatgggtcactacttgtgatttccgctggtatggaccgaagtatataacacattccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaaagggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaagtaa

SEQ ID NO: 180 - protein, 422 aa, synthetic construct
MSVTVKRIIDNTVVVPKLPANEDPVEYPADYFRKSKEIPLYINTTKSLSDLRGYVYQGLKSGNVSIIHVNSYLYGALKDIRGKLDKDWSSFGINIGKAGDTIGIFDLVSLKALDGVLPDGVSDASRTSADDKWLPLYLLGLYRVGRTQMPEYRKKLMDGLTNQCKMINEQFEPLVPEGRDIFDVWGNDSNYTKIVAAVDMFFHMFKKHECASFRYGTIVSRFKDCAALATFGHLCKITGMSTEDVTTWILNREVADEMVQMMLPGQEIDKADSYMPYLIDFGLSSKSPYSSVKNPAFHFWGQLTALLLRSTRARNARQPDDIEYTSLTTAGLLYAYAVGSSADLAQQFCVGDNKYTPDDSTGGLTTNAPPQGRDVVEWLGWFEDQNRKPTPDMMQYAKRAVMSLQGLREKTIGKYAKSEFDK

SEQ ID NO: 181 - DNA, 1269 bp, synthetic construct
  source 1..1269: mol_type = other DNA
atgtctgttacagtcaagagaatcattgacaacacagtcgtagttccaaaacttcctgcaaatgaggatccagtggaatacccggcagattacttcagaaaatcaaaggagattcctctttacatcaatactacaaaaagtttgtcagatctaagaggatatgtctaccaaggcctcaaatccggaaatgtatcaatcatacatgtcaacagctacttgtatggagcattaaaggacatccggggtaagttggataaagattggtcaagtttcggaataaacatcgggaaagcaggggatacaatcggaatatttgaccttgtatccttgaaagccctggacggcgtacttccagatggagtatcggatgcttccagaaccagcgcagatgacaaatggttgcctttgtatctacttggcttatacagagtgggcagaacacaaatgcctgaatacagaaaaaagctcatggatgggctgacaaatcaatgcaaaatgatcaatgaacagtttgaacctcttgtgccagaaggtcgtgacatttttgatgtgtggggaaatgacagtaattacacaaaaattgtcgctgcagtggacatgttcttccacatgttcaaaaaacatgaatgtgcctcgttcagatacggaactattgtttccagattcaaagattgtgctgcattggcaacatttggacacctctgcaaaataaccggaatgtctacagaagatgtaacgacctggatcttgaaccgagaagttgcagatgaaatggtccaaatgatgcttccaggccaagaaattgacaaggccgattcatacatgccttatttgatcgactttggattgtcttctaagtctccatattcttccgtcaaaaaccctgccttccacttctgggggcaattgacagctcttctgctcagatccaccagagcaaggaatgcccgacagcctgatgacattgagtatacatctcttactacagcaggtttgttgtacgcttatgcagtaggatcctctgccgacttggcacaacagttttgtgttggagataacaaatacactccagatgatagtaccggaggattgacgactaatgcaccgccacaaggcagagatgtggtcgaatggctcggatggtttgaagatcaaaacagaaaaccgactcctgatatgatgcagtatgcgaaaagagcagtcatgtcactgcaaggcctaagagagaagacaattggcaagtatgctaagtcagaatttgacaaatga

SEQ ID NO: 182 - protein, 265 aa, synthetic construct
MDNLTKVREYLKSYSRLDQAVGEIDEIEAQRAEKSNYELFQEDGVEEHTKPSYFQAADDSDTESEPEIEDNQGLYAPDPEAEQVEGFIQGPLDDYADEEVDVVFTSDWKQPELESDEHGKTLRLTSPEGLSGEQKSQWLSTIKAVVQSAKYWNLAECTFEASGEGVIMKERQITPDVYKVTPVMNTHPSQSEAVSDVWSLSKTSMTFQPKKASLQPLTISLDELFSSRGEFISVGGDGRMSHKEAILLGLRYKKLYNQARVKYSL

SEQ ID NO: 183 - DNA, 798 bp, synthetic construct
  source 1..798: mol_type = other DNA
atggataatctcacaaaagttcgtgagtatctcaagtcctattctcgtctggatcaggcggtaggagagatagatgagatcgaagcacaacgagctgaaaagtccaattatgagttgttccaagaggatggagtggaagagcatactaagccctcttattttcaggcagcagatgattctgacacagaatctgaaccagaaattgaagacaatcaaggcttgtatgcaccagatccagaagcagagcaagttgaaggctttatacaggggcctttagatgactatgcagatgaggaagtggatgttgtatttacttcggactggaaacagcctgagcttgaatctgacgagcatggaaagaccttacggttgacatcgccagagggtttaagtggagagcagaaatcccagtggctttcgacgattaaagcagtcgtgcaaagtgccaaatactggaatctggcagagtgcacatttgaagcatcgggagaaggggtcattatgaaggagcgccagataactccggatgtatataaggtcactccagtgatgaacacacatccgtcccaatcagaagcagtatcagatgtttggtctctctcaaagacatccatgactttccaacccaagaaagcaagtcttcagcctctcaccatatccttggatgaattgttctcatctagaggagagttcatctctgtcggaggtgacggacgaatgtctcataaagaggccatcctgctcggcctgagatacaaaaagttgtacaatcaggcgagagtcaaatattctctgtag

SEQ ID NO: 184 - protein, 2109 aa, synthetic construct
MEVHDFETDEFNDFNEDDYATREFLNPDERMTYLNHADYNLNSPLISDDIDNLIRKFNSLPIPSMWDSKNWDGVLEMLTSCQANPIPTSQMHKWMGSWLMSDNHDASQGYSFLHEVDKEAEITFDVVETFIRGWGNKPIEYIKKERWTDSFKILAYLCQKFLDLHKLTLILNAVSEVELLNLARTFKGKVRRSSHGTNICRIRVPSLGPTFISEGWAYFKKLDILMDRNFLLMVKDVIIGRMQTVLSMVCRIDNLFSEQDIFSLLNIYRIGDKIVERQGNFSYDLIKMVEPICNLKLMKLARESRPLVPQFPHFENHIKTSVDEGAKIDRGIRFLHDQIMSVKTVDLTLVIYGSFRHWGHPFIDYYTGLEKLHSQVTMKKDIDVSYAKALASDLARIVLFQQFNDHKKWFVNGDLLPHDHPFKSHVKENTWPTAAQVQDFGDKWHELPLIKCFEIPDLLDPSIIYSDKSHSMNRSEVLKHVRMNPNTPIPSKKVLQTMLDTKATNWKEFLKEIDEKGLDDDDLIIGLKGKERELKLAGRFFSLMSWKLREYFVITEYLIKTHFVPMFKGLTMADDLTAVIKKMLDSSSGQGLKSYEAICIANHIDYEKWNNHQRKLSNGPVFRVMGQFLGYPSLIERTHEFFEKSLIYYNGRPDLMRVHNNTLINSTSQRVCWQGQEGGLEGLRQKGWSILNLLVIQREAKIRNTAVKVLAQGDNQVICTQYKTKKSRNVVELQGALNQMVSNNEKIMTAIKIGTGKLGLLINDDETMQSADYLNYGKIPIFRGVIRGLETKRWSRVTCVTNDQIPTCANIMSSVSTNALTVAHFAENPINAMIQYNYFGTFARLLLMMHDPALRQSLYEVQDKIPGLHSSTFKYAMLYLDPSIGGVSGMSLSRFLIRAFPDPVTESLSFWRFIHVHARSEHLKEMSAVFGNPEIAKFRITHIDKLVEDPTSLNIAMGMSPANLLKTEVKKCLIESRQTIRNQVIKDATIYLYHEEDRLRSFLWSINPLFPRFLSEFKSGTFLGVADGLISLFQNSRTIRNSFKKKYHRELDDLIVRSEVSSLTHLGKLHLRRGSCKMWTCSATHADTLRYKSWGRTVIGTTVPHPLEMLGPQHRKETPCAPCNTSGFNYVSVHCPDGIHDVFSSRGPLPAYLGSKTSESTSILQPWERESKVPLIKRATRLRDAISWFVEPDSKLAMTILSNIHSLTGEEWTKRQHGFKRTGSALHRFSTSRMSHGGFASQSTAALTRLMATTDTMRDLGDQNFDFLFQATLLYAQITTTVARDGWITSCTDHYHIACKSCLRPIEEITLDSSMDYTPPDVSHVLKTWRNGEGSWGQEIKQIYPLEGNWKNLAPAEQSYQVGRCIGFLYGDLAYRKSTHAEDSSLFPLSIQGRIRGRGFLKGLLDGLMRASCCQVIHRRSLAHLKRPANAVYGGLIYLIDKLSVSPPFLSLTRSGPIRDELETIPHKIPTSYPTSNRDMGVIVRNYFKYQCRLIEKGKYRSHYSQLWLFSDVLSIDFIGPFSISTTLLQILYKPFLSGKDKNELRELANLSSLLRSGEGWEDIHVKFFTKDILLCPEEIRHACKFGIAKDNNKDMSYPPWGRESRGTITTIPVYYTTTPYPKMLEMPPRIQNPLLSGIRLGQLPTGAHYKIRSILHGMGIHYRDFLSCGDGSGGMTAALLRENVHSRGIFNSLLELSGSVMRGASPEPPSALETLGGDKSRCVNGETCWEYPSDLCDPRTWDYFLRLKAGLGLQIDLIVMDMEVRDSSTSLKIETNVRNYVHRILDEQGVLIYKTYGTYICESEKNAVTILGPMFKTVDLVQTEFSSSQTSEVYMVCKGLKKLIDEPNPDWSSINESWKNLYAFQSSEQEFARAKKVSTYFTLTGIPSQFIPDPFVNIETMLQIFGVPTGVSHAAALKSSDRPADLLTISLFYMAIISYYNINHIRVGPIPPNPPSDGIAQNVGIAITGISFWLSLMEKDIPLYQQCLAVIQQSFPIRWEAVSVKGGYKQKWSTRGDGLPKDTRISDSLAPIGNWIRSLELVRNQVRLNPFNEILFNQLCRTVDNHLKWSNLRRNTGMIEWINRRISKEDRSILMLKSDLHEENSWRD

SEQ ID NO: 185 - DNA, 6330 bp, synthetic construct
  source 1..6330: mol_type = other DNA
atggaagtccacgattttgagaccgacgagttcaatgatttcaatgaagatgactatgccacaagagaattcctgaatcccgatgagcgcatgacgtacttgaatcatgctgattacaacctgaattctcctctaattagtgatgatattgacaatttaatcaggaaattcaattctcttccaattccctcgatgtgggatagtaagaactgggatggagttcttgagatgttaacgtcatgtcaagccaatcccatcccaacatctcagatgcataaatggatgggaagttggttaatgtctgataatcatgatgccagtcaagggtatagttttttacatgaagtggacaaagaggcagaaataacatttgacgtggtggagaccttcatccgcggctggggcaacaaaccaattgaatacatcaaaaaggaaagatggactgactcattcaaaattctcgcttatttgtgtcaaaagtttttggacttacacaagttgacattaatcttaaatgctgtctctgaggtggaattgctcaacttggcgaggactttcaaaggcaaagtcagaagaagttctcatggaacgaacatatgcaggattagggttcccagcttgggtcctacttttatttcagaaggatgggcttacttcaagaaacttgatattctaatggaccgaaactttctgttaatggtcaaagatgtgattatagggaggatgcaaacggtgctatccatggtatgtagaatagacaacctgttctcagagcaagacatcttctcccttctaaatatctacagaattggagataaaattgtggagaggcagggaaatttttcttatgacttgattaaaatggtggaaccgatatgcaacttgaagctgatgaaattagcaagagaatcaaggcctttagtcccacaattccctcattttgaaaatcatatcaagacttctgttgatgaaggggcaaaaattgaccgaggtataagattcctccatgatcagataatgagtgtgaaaacagtggatctcacactggtgatttatggatcgttcagacattggggtcatccttttatagattattacactggactagaaaaattacattcccaagtaaccatgaagaaagatattgatgtgtcatatgcaaaagcacttgcaagtgatttagctcggattgttctatttcaacagttcaatgatcataaaaagtggttcgtgaatggagacttgctccctcatgatcatccctttaaaagtcatgttaaagaaaatacatggcccacagctgctcaagttcaagattttggagataaatggcatgaacttccgctgattaaatgttttgaaatacccgacttactagacccatcgataatatactctgacaaaagtcattcaatgaataggtcagaggtgttgaaacatgtccgaatgaatccgaacactcctatccctagtaaaaaggtgttgcagactatgttggacacaaaggctaccaattggaaagaatttcttaaagagattgatgagaagggcttagatgatgatgatctaattattggtcttaaaggaaaggagagggaactgaagttggcaggtagatttttctccctaatgtcttggaaattgcgagaatactttgtaattaccgaatatttgataaagactcatttcgtccctatgtttaaaggcctgacaatggcggacgatctaactgcagtcattaaaaagatgttagattcctcatccggccaaggattgaagtcatatgaggcaatttgcatagccaatcacattgattacgaaaaatggaataaccaccaaaggaagttatcaaacggcccagtgttccgagttatgggccagttcttaggttatccatccttaatcgagagaactcatgaattttttgagaaaagtcttatatactacaatggaagaccagacttgatgcgtgttcacaacaacacactgatcaattcaacctcccaacgagtttgttggcaaggacaagagggtggactggaaggtctacggcaaaaaggatggagtatcctcaatctactggttattcaaagagaggctaaaatcagaaacactgctgtcaaagtcttggcacaaggtgataatcaagttatttgcacacagtataaaacgaagaaatcgagaaacgttgtagaattacagggtgctctcaatcaaatggtttctaataatgagaaaattatgactgcaatcaaaatagggacagggaagttaggacttttgataaatgacgatgagactatgcaatctgcagattacttgaattatggaaaaataccgattttccgtggagtgattagagggttagagaccaagagatggtcacgagtgacttgtgtcaccaatgaccaaatacccacttgtgctaatataatgagctcagtttccacaaatgctctcaccgtagctcattttgctgagaacccaatcaatgccatgatacagtacaattattttgggacatttgctagactcttgttgatgatgcatgatcctgctcttcgtcaatcattgtatgaagttcaagataagataccgggcttgcacagttctactttcaaatacgccatgttgtatttggacccttccattggaggagtgtcgggcatgtctttgtccaggtttttgattagagccttcccagatcccgtaacagaaagtctctcattctggagattcatccatgtacatgctcgaagtgagcatctgaaggagatgagtgcagtatttggaaaccccgagatagccaagtttcgaataactcacatagacaagctagtagaagatccaacctctctgaacatcgctatgggaatgagtccagcgaacttgttaaagactgaggttaaaaaatgcttaatcgaatcaagacaaaccatcaggaaccaggtgattaaggatgcaaccatatatttgtatcatgaagaggatcggctcagaagtttcttatggtcaataaatcctctgttccctagatttttaagtgaattcaaatcaggcacttttttgggagtcgcagacgggctcatcagtctatttcaaaattctcgtactattcggaactcctttaagaaaaagtatcatagggaattggatgatttgattgtgaggagtgaggtatcctctttgacacatttagggaaacttcatttgagaaggggatcatgtaaaatgtggacatgttcagctactcatgctgacacattaagatacaaatcctggggccgtacagttattgggacaactgtaccccatccattagaaatgttgggtccacaacatcgaaaagagactccttgtgcaccatgtaacacatcagggttcaattatgtttctgtgcattgtccagacgggatccatgacgtctttagttcacggggaccattgcctgcttatctagggtctaaaacatctgaatctacatctattttgcagccttgggaaagggaaagcaaagtcccactgattaaaagagctacacgtcttagagatgctatctcttggtttgttgaacccgactctaaactagcaatgactatactttctaacatccactctttaacaggcgaagaatggaccaaaaggcagcatgggttcaaaagaacagggtctgcccttcataggttttcgacatctcggatgagccatggtgggttcgcatctcagagcactgcagcattgaccaggttgatggcaactacagacaccatgagggatctgggagatcagaatttcgactttttattccaagcaacgttgctctatgctcaaattaccaccactgttgcaagagacggatggatcaccagttgtacagatcattatcatattgcctgtaagtcctgtttgagacccatagaagagatcaccctggactcaagtatggactacacgcccccagatgtatcccatgtgctgaagacatggaggaatggggaaggttcgtggggacaagagataaaacagatctatcctttagaagggaattggaagaatttagcacctgcagagcaatcctatcaagtcggcagatgtataggttttctatatggagacttggcgtatagaaaatctactcatgccgaggacagttctctatttcctctatctatacaaggtcgtattagaggtcgaggtttcttaaaagggttgctagacggattaatgagagcaagttgctgccaagtaatacaccggagaagtctggctcatttgaagaggccggccaacgcagtgtacggaggtttgatttacttgattgataaattgagtgtatcacctccattcctttctcttactagatcaggacctattagagacgaattagaaacgattccccacaagatcccaacctcctatccgacaagcaaccgtgatatgggggtgattgtcagaaattacttcaaataccaatgccgtctaattgaaaagggaaaatacagatcacattattcacaattatggttattctcagatgtcttatccatagacttcattggaccattctctatttccaccaccctcttgcaaatcctatacaagccatttttatctgggaaagataagaatgagttgagagagctggcaaatctttcttcattgctaagatcaggagaggggtgggaagacatacatgtgaaattcttcaccaaggacatattattgtgtccagaggaaatcagacatgcttgcaagttcgggattgctaaggataataataaagacatgagctatcccccttggggaagggaatccagagggacaattacaacaatccctgtttattatacgaccaccccttacccaaagatgctagagatgcctccaagaatccaaaatcccctgctgtccggaatcaggttgggccaattaccaactggcgctcattataaaattcggagtatattacatggaatgggaatccattacagggacttcttgagttgtggagacggctccggagggatgactgctgcattactacgagaaaatgtgcatagcagaggaatattcaatagtctgttagaattatcagggtcagtcatgcgaggcgcctctcctgagccccccagtgccctagaaactttaggaggagataaatcgagatgtgtaaatggtgaaacatgttgggaatatccatctgacttatgtgacccaaggacttgggactatttcctccgactcaaagcaggcttggggcttcaaattgatttaattgtaatggatatggaagttcgggattcttctactagcctgaaaattgagacgaatgttagaaattatgtgcaccggattttggatgagcaaggagttttaatctacaagacttatggaacatatatttgtgagagcgaaaagaatgcagtaacaatccttggtcccatgttcaagacggtcgacttagttcaaacagaatttagtagttctcaaacgtctgaagtatatatggtatgtaaaggtttgaagaaattaatcgatgaacccaatcccgattggtcttccatcaatgaatcctggaaaaacctgtacgcattccagtcatcagaacaggaatttgccagagcaaagaaggttagtacatactttaccttgacaggtattccctcccaattcattcctgatccttttgtaaacattgagactatgctacaaatattcggagtacccacgggtgtgtctcatgcggctgccttaaaatcatctgatagacctgcagatttattgaccattagccttttttatatggcgattatatcgtattataacatcaatcatatcagagtaggaccgatacctccgaaccccccatcagatggaattgcacaaaatgtggggatcgctataactggtataagcttttggctgagtttgatggagaaagacattccactatatcaacagtgtttagcagttatccagcaatcattcccgattaggtgggaggctgtttcagtaaaaggaggatacaagcagaagtggagtactagaggtgatgggctcccaaaagatacccgaatttcagactccttggccccaatcgggaactggatcagatctctggaattggtccgaaaccaagttcgtctaaatccattcaatgagatcttgttcaatcagctatgtcgtacagtggataatcatttgaaatggtcaaatttgcgaagaaacacaggaatgattgaatggatcaatagacgaatttcaaaagaagaccggtctatactgatgttgaagagtgacctacacgaggaaaactcttggagagattaa

SEQ ID NO: 186 - protein, 53 aa, synthetic construct
NSYSECPPSYDGYCLHDGVCRYIEALDSYACNCVVGYAGERCQYRDLRWWGRR

SEQ ID NO: 187 - protein, 127 aa, synthetic construct
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSALE

SEQ ID NO: 188 - protein, 130 aa, synthetic construct
EVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSSALE

SEQ ID NO: 189 - protein, 165 aa, Homo sapiens
EGICRNRVTNNVKDVTKLVANLPKDYMITLKYVPGMDVLPSHCWISEMVVQLSDSLTDLLDKFSNISEGLSNYSIIDKLVNIVDDLVECVKENSSKDLKKSFKSPEPRLFTPEEFFRIFNRSIDAFKDFVVASETSDCVVSSTLSPEKDSRVSVTKPFMLPPVAA

SEQ ID NO: 190 - protein, 70 aa, Homo sapiens
GPETLCGAELVDALQFVCGDRGFYFNKPTGYGSSSRRAPQTGIVDECCFRSCDLRRLEMYCAPLKPAKSA

SEQ ID NO: 191 - protein, 27 aa, synthetic construct
NKFNKGMRGYWGALGGGNGKRGIMGYD

SEQ ID NO: 192 - protein, 255 aa, synthetic construct
QVQLLQSGAELKKPGESLKISCKGSGYSFTSYWIAWVRQMPGKGLEYMGLIYPGDSDTKYSPSFQGQVTISVDKSVSTAYLQWSSLKPSDSAVYFCARHDVGYCSSSNCAKWPEYFQHWGQGTLVTVSSGGGGSGGGGSGGGGSQSVLTQPPSVSAAPGQKVTISCSGSSSNIGNNYVSWYQQLPGTAPKLLIYGHTNRPAGVPDRFSGSKSGTSASLAISGFRSEDEADYYCAAWDDSLSGWVFGGGTKLTVLG

SEQ ID NO: 193 - protein, 129 aa, synthetic construct
QVQLLQSGAELKKPGESLKISCKGSGYSFTSYWIAWVRQMPGKGLEYMGLIYPGDSDTKYSPSFQGQVTISVDKSVSTAYLQWSSLKPSDSAVYFCARHDVGYCSSSNCAKWPEYFQHWGQGTLVTVSS

SEQ ID NO: 194 - protein, 111 aa, synthetic construct
QSVLTQPPSVSAAPGQKVTISCSGSSSNIGNNYVSWYQQLPGTAPKLLIYGHTNRPAGVPDRFSGSKSGTSASLAISGFRSEDEADYYCAAWDDSLSGWVFGGGTKLTVLG

SEQ ID NO: 195 - protein, 245 aa, synthetic construct
QVKLQESGGGLVQPGGSLRLSCAASGFTFDSYAMSWVRQAPGKGLEWVSYITSSSSTIYYVDSVKGRFTISRDNAKNSLYLQMNSLRDEDTAVYYCARLRNSEGYWYFDLWGRGTLVTVSSGGGGSGGGGSGGGGSQSALTQDPAVSVALGQTVRITCQGDSLRSYFASWYQQKPGQAPLLVMYGQNIRPSGIPDRFSGSSSGNSASLTITGAQAEDEADYYCNSRDSSYNHWVFGGGTKLTVLG

SEQ ID NO: 196 - protein, 121 aa, synthetic construct
QVKLQESGGGLVQPGGSLRLSCAASGFTFDSYAMSWVRQAPGKGLEWVSYITSSSSTIYYVDSVKGRFTISRDNAKNSLYLQMNSLRDEDTAVYYCARLRNSEGYWYFDLWGRGTLVTVSS

SEQ ID NO: 197 - protein, 109 aa, synthetic construct
QSALTQDPAVSVALGQTVRITCQGDSLRSYFASWYQQKPGQAPLLVMYGQNIRPSGIPDRFSGSSSGNSASLTITGAQAEDEADYYCNSRDSSYNHWVFGGGTKLTVLG

SEQ ID NO: 198-204 - skipped (7 entries)

SEQ ID NO: 205 - protein, 15 aa, synthetic construct
EGGGKEGGGKEGGGK

SEQ ID NO: 206 - protein, 17 aa, synthetic construct
KREGGGKEGGGKEGGGK

SEQ ID NO: 207 - protein, 16 aa, synthetic construct
REGGGKEGGGKEGGGK

SEQ ID NO: 208 - protein, 23 aa, synthetic construct
GGGRGSPKGGGGSGGGGSGGGGS

SEQ ID NO: 209 - protein, 24 aa, synthetic construct
RGGGRGSPKGGGGSGGGGSGGGGS

SEQ ID NO: 210 - protein, 24 aa, synthetic construct
GGGRGSPKGGGGSGGGGSGGGGSK

SEQ ID NO: 211 - protein, 19 aa, synthetic construct
GGGGGGGSGGGGSGGGGSK

SEQ ID NO: 212 - protein, 18 aa, synthetic construct
GGGGGGGSGGGGSGGGGS

SEQ ID NO: 213 - DNA, 123 bp, synthetic construct
  source 1..123: mol_type = other DNA
tggcataatgacttaataggcacagccttacaagtcaaaatgcccaagagtcacaaggctattcaagcagacggttggatgtgtcatgcttccaaatgggtcactacttgtgatttccgctgg

SEQ ID NO: 214 - DNA, 123 bp, synthetic construct
  source 1..123: mol_type = other DNA
accgtattactgaattatccgtgtcggaatgttcagttttacgggttctcagtgttccgataagttcgtctgccaacctacacagtacgaaggtttacccagtgatgaacactaaaggcgacc

SEQ ID NO: 215 - protein, 41 aa, synthetic construct
WHNDLIGTALQVKMPKSHKAIQADGWMCHASKWVTTCDFRW

SEQ ID NO: 216 - protein, 31 aa, synthetic construct
LDFHGLTVLSNLCVTPHTMSGFPDSSTIEAP

SEQ ID NO: 217 - DNA, 120 bp, synthetic construct
  source 1..120: mol_type = other DNA
tggcataatgacttaataggcacagccttacaagtcaaaatgcccagtcacaaggctattcaagcagacggttggatgtgtcatgcttccaaatgggtcactacttgtgatttccgctgg

SEQ ID NO: 218 - DNA, 120 bp, synthetic construct
  source 1..120: mol_type = other DNA
accgtattactgaattatccgtgtcggaatgttcagttttacgggtcagtgttccgataagttcgtctgccaacctacacagtacgaaggtttacccagtgatgaacactaaaggcgacc

SEQ ID NO: 219 - protein, 40 aa, synthetic construct
WHNDLIGTALQVKMPSHKAIQADGWMCHASKWVTTCDFRW

SEQ ID NO: 220 - protein, 30 aa, synthetic construct
LDFHGTVLSNLCVTPHTMSGFPDSSTIEAP

SEQ ID NO: 221 - DNA, 118 bp, synthetic construct
  source 1..118: mol_type = other DNA
attggacatggtatgttggactccgatcttcatcttagctcaaaggctcaggtgttcgaacatcctcacattcaagacgctgcttcgcaacttcctgatgatgagagtttattttttg

SEQ ID NO: 222 - DNA, 118 bp, synthetic construct
  source 1..118: mol_type = other DNA
taacctgtaccatacaacctgaggctagaagtagaatcgagtttccgagtccacaagcttgtaggagtgtaagttctgcgacgaagcgttgaaggactactactctcaaataaaaaac

SEQ ID NO: 223 - protein, 39 aa, synthetic construct
IGHGMLDSDLHLSSKAQVFEHPHIQDAASQLPDDESLFF

SEQ ID NO: 224 - DNA, 118 bp, synthetic construct
  source 1..118: mol_type = other DNA
attggacatggtatgttggactccgatcttcatcttagctcaaaggctcaggtgatcgaacatcctcacattcaagacgctgcttcgcaacttcctgatgatgagagtttattttttg

SEQ ID NO: 225 - DNA, 118 bp, synthetic construct
  source 1..118: mol_type = other DNA
taacctgtaccatacaacctgaggctagaagtagaatcgagtttccgagtccactagcttgtaggagtgtaagttctgcgacgaagcgttgaaggactactactctcaaataaaaaac

SEQ ID NO: 226 - protein, 39 aa, synthetic construct
IGHGMLDSDLHLSSKAQVIEHPHIQDAASQLPDDESLFF

SEQ ID NO: 227 - protein, 4 aa, synthetic construct
PPPY

SEQ ID NO: 228 - protein, 4 aa, synthetic construct
APPY

SEQ ID NO: 229 - protein, 4 aa, synthetic construct
AAPY

SEQ ID NO: 230 - protein, 4 aa, synthetic construct
PPAY

SEQ ID NO: 231 - protein, 4 aa, synthetic construct
APPA

SEQ ID NO: 232 - protein, 4 aa, synthetic construct
AAPA

SEQ ID NO: 233 - protein, 4 aa, synthetic construct
PPPA

SEQ ID NO: 234 - protein, 4 aa, synthetic construct
PSAP

SEQ ID NO: 235 - protein, 6 aa, synthetic construct
HHHHHH

SEQ ID NO: 236 - protein, 16 aa, synthetic construct
CPPCPAPELLGGPSVF